(19) United States
(12) Patent Application Publication
(10) Pub. No.: US 2025/0093263 A1
(43) Pub. Date: Mar. 20, 2025

(54) FRET-BASED ASSAYS

(71) Applicants: The General Hospital Corporation, Boston, MA (US); PRESIDENT AND FELLOWS OF HARVARD COLLEGE, Cambridge, MA (US)

(72) Inventors: Ralph ` Mazitschek, Weston, MA (US); N. Connor Payne, Boston, MA (US); Mark A. Tye, San Ramon, CA (US)

(21) Appl. No.: 18/580,597

(22) PCT Filed: Jul. 22, 2022
(86) PCT No.: PCT/US2022/074087

§ 371 Date: Jan. 18, 2024

Related U.S. Application Data
(60) Provisional application No. 63/224,433, filed on Jul. 22, 2021.
(60) Provisional application No. 63/277,643, filed on Nov. 10, 2021.
(60) Provisional application No. 63/333,080, filed on Apr. 20, 2022.
(60) Provisional application No. 63/353,348, filed on Jun. 17, 2022.
(60) Provisional application No. 63/353,526, filed on Jun. 17, 2022.
(60) Provisional application No. 63/391,655, filed on Jul. 22, 2022.

(51) Int. Cl. G01N 21/64; A61K 31/497; C07D 401/12; C07D 401/14; C07D 487/04; G01N 33/573; G01N 33/58

(52) U.S. Cl. CPC G01N 21/6428; A61K 31/497; C07D 401/12; C07D 401/14; C07D 487/04; G01N 33/573; G01N 33/582; G01N 2021/6439; G01N 2333/9015

(57) ABSTRACT

The present application provides compounds and methods for identifying a modulator of a protein of interest using TR-FRET donor attached to the protein of interest and a tracer containing TR-FRET acceptor. Iterations of this compound may also includes attachment of an ATP-binding moiety, an amino acid, and/or a fluorophore.

### CLAIM OF PRIORITY

This application claims priority to U.S. Provisional Patent Application Ser. No. 63/224,433, filed on Jul. 22, 2021, to U.S. Provisional Patent Application Ser. No. 63/277,643, filed on Nov. 10, 2011, to U.S. Provisional Patent Application Ser. No. 63/333,080, filed on Apr. 20, 2022, U.S. Provisional Patent Application Ser. No. 63/353,348, filed on Jun. 17, 2022, to U.S. Provisional Patent Application Ser. No. 63/353,526, filed on Jun. 17, 2022, and to U.S. Provisional Patent Application Ser. No. 63/391,655, filed on Jul. 22, 2022, the entire contents of which are hereby incorporated by reference.

### TECHNICAL FIELD

This disclosure relates to assays to study or identify modulators (e.g., small-molecule modulators) of proteins of interest, and in particular to assays utilizing the Förster resonance energy transfer (FRET) between a donor and a acceptor that are bound or otherwise associated with the protein of interest.

### BACKGROUND

The pace of progress in biomedical research directly depends on techniques that allow for reliable, quantitative, sensitive, fast, inexpensive, miniaturized and massively parallel experimental designs, such as next-generation sequencing and high-throughput-screening (HTS). The lack of such robust and straightforward assays for disease-relevant targets of interest can significantly hamper the discovery and development of inhibitors that could potentially yield first-in-class therapeutics and address unmet medical needs. An ideal assay platform enables the direct and quantitative measurement of the interaction between a ligand (e.g. small molecules, peptides or other biomolecules such as carbohydrates, lipids and nucleic acids) with a target protein of interest (POI), offers the flexibility to determine both thermodynamic and kinetic binding constants, and provides information on the binding modality (e.g. substrate competitiveness). In one example, a target POI is expressed recombinantly as a fusion protein with an epitope tag to facilitate purification and/or enable specific labeling and detection (e.g. His6-tag, GST-tag, Flag-tag, Halo Tag). Such modifications not only greatly simplify protein production but can also be advantageous for assay development. However, sometimes the expression of epitope-tag fusion proteins is unsuccessful, or the epitope tag can interfere with protein function. In these instances, it can be necessary to assay the native, unmodified wild-type POI. Moreover, in some instances, the POI is a member of one or more defined multi-protein complexes and may exhibit differential affinities for small molecule ligands depending on the specific complex a POI resides in. In these instances, the ability to selectively profile compounds for the POI in a specific complex type can be highly desirable but can be difficult to accomplish, particularly in the presence of other complex types carrying the POI. Furthermore, in some instances (e.g., following drug treatment) it might be desirable to assay/quantify the abundance of total POI or POI-specific post-translations modifications (e.g. acetylation, phosphorylation or glycosylation) in cells or in cell lysates in time and dose-dependent manner.

### SUMMARY

Disclosed herein are assay platforms that utilize TR-FRET donors (e.g., CoraFluors) as well as small-molecule ligands labeled with a FRET-acceptor (e.g., a fluorophore) that are both capable of binding to a protein of interest. These platforms address unmet needs and greatly enable drug development efforts for various pharmacologically relevant targets. Small molecule modulators (e.g., inhibitors, activators, molecular glues) of proteins of interest (biological targets relevant to human health and disease) that were discovered on the basis of the novel assay platforms are also disclosed. Examples of the proteins of interest include aminoacyl tRNA synthesizes, specific histone deacetylase and histone deacetylase complexes, G-protein coupled receptors, and cysteine-rich proteins such as Keap1. The assays disclosed herein greatly simplify existing approaches, while improving sensitivity, flexibility, robustness and throughput. These assays enable, for example, a very sensitive high-throughput screening (HTS) for small-molecule drug candidates. The straightforward, single-step biochemical assay platform not only facilitates HTS, but also allows reliable ligand characterization, including kinetic and substrate-dependent profiling with accurate determination of binding affinities, binding kinetics, and mode of protein modulation (e.g., substrate-independent inhibition). The assays within the instant claims offer a unique advantage over existing assay platforms. The assay may be performed, for example, with as little as picomolar concentration of the protein of interest, which is >1,000 and 100-fold lower than current non-radioactive and radioactive assay platforms, respectively. Importantly, the assay platform also allows to use live cell applications, which is not possible with current TR-FRET technologies.

In some embodiments, the present disclosure provides a compound of Formula (I):

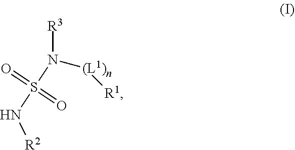

or a pharmaceutically acceptable salt thereof.

In some embodiments, the present disclosure provides a compound of Formula (I):

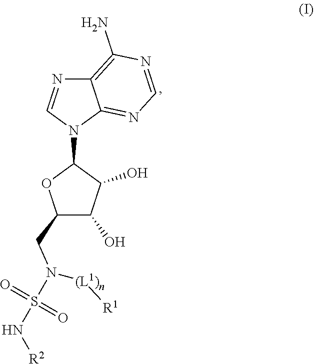

or a pharmaceutically acceptable salt thereof.

In some embodiments, this disclosure provides a compound of Formula (II):

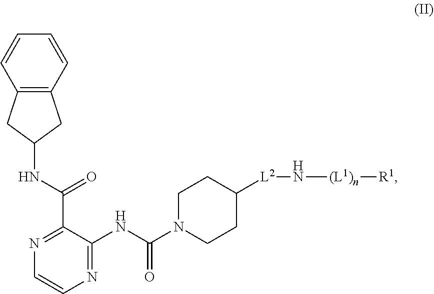

or a pharmaceutically acceptable salt thereof.

In some embodiments, the present disclosure provides a compound of Formula (III):

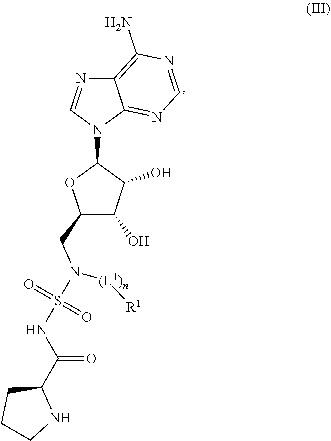

or a pharmaceutically acceptable salt thereof.

In some embodiments, the present disclosure provides a compound of Formula (IV):

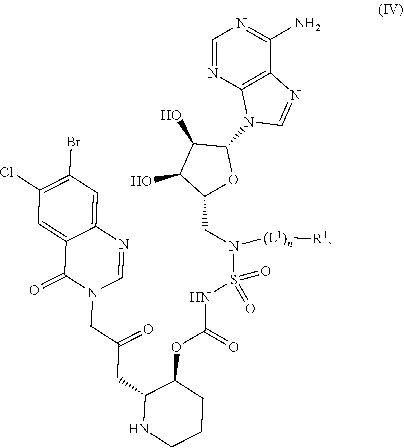

or a pharmaceutically acceptable salt thereof.

In some embodiments, the present disclosure provides a compound of Formula (V):

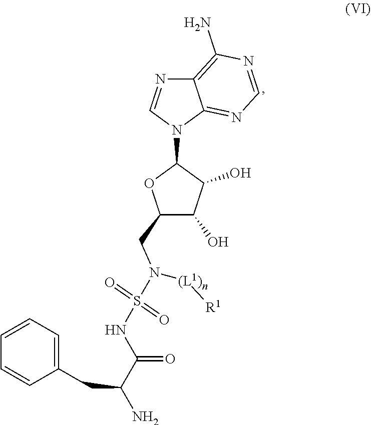

or a pharmaceutically acceptable salt thereof.

In some embodiments, the present disclosure provides a compound of Formula (VI):

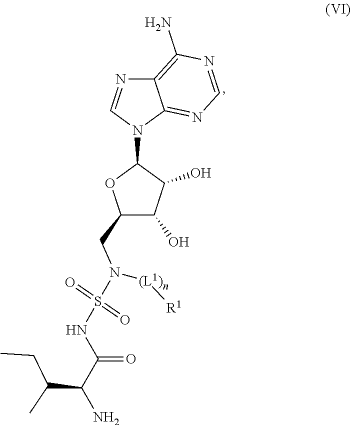

or a pharmaceutically acceptable salt thereof.

In some embodiments, the present disclosure provides a compound of Formula (VII):

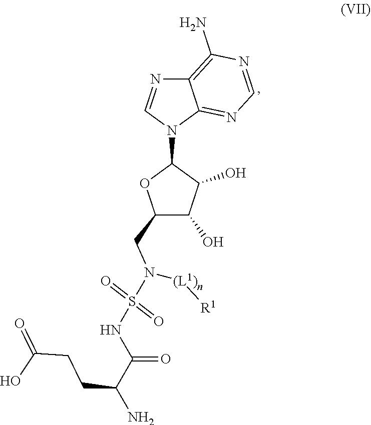

or a pharmaceutically acceptable salt thereof.

In some embodiments, the present disclosure provides a compound of Formula (A):

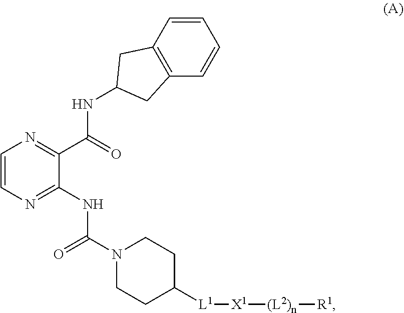

or a pharmaceutically acceptable salt thereof.

In some embodiments, the present disclosure provides a compound of Formula (B):

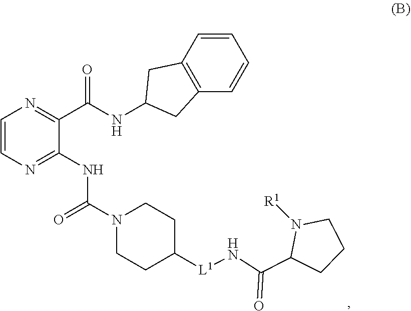

or a pharmaceutically acceptable salt thereof.

In some embodiments, the present disclosure provides a pharmaceutical composition comprising a compound of Formula (B), or a pharmaceutically acceptable salt thereof, and a pharmaceutically acceptable carrier.

In some embodiments, the present disclosure provides a method of inhibiting prolyl-tRNA-synthetase in a cell, comprising contacting the cell with a compound of Formula (B), or a pharmaceutically acceptable salt thereof.

In some embodiments, the present disclosure provides a method of inhibiting prolyl-tRNA-synthetase in a subject, comprising administering to the subject a compound of Formula (B), or a pharmaceutically acceptable salt thereof.

In some embodiments, the present disclosure provides a method of treating a disorder associated with glutamyl-prolyl-tRNA synthetase, prolyl-tRNA synthetase, or a combination thereof, the method comprising administering to a subject in need thereof a therapeutically effective amount of a compound of Formula (B), or a pharmaceutically acceptable salt thereof, and a pharmaceutically acceptable carrier.

In some embodiments, the present disclosure provides a method of identifying a compound that modulates a protein of interest, the method comprising:
(i) providing a sample comprising a protein of interest attached to a FRET donor moiety and a ligand attached to a FRET acceptor moiety, said ligand capable of binding to the protein of interest;
(ii) exciting the sample with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique;
(iii) contacting the sample with a test compound;
(iv) after (iii), detecting a light of a wavelength emitted by the FRET acceptor moiety by a fluorescence imaging technique; and
(v) determining whether the intensity of fluorescence detected from the FRET acceptor moiety in step (iv) is decreased compared to the intensity of fluorescence detected from the FRET acceptor moiety in step (ii),
wherein said decrease in fluorescence intensity in an indication that the test compound is the modulator the protein of interest.

In some embodiments, the present disclosure provides a method of evaluating an interaction between a protein of interest and a modulator of the protein of interest, the method comprising:
(i) providing a sample comprising the protein of interest attached to a FRET donor moiety and the modulator attached to a FRET acceptor moiety;
(ii) exciting the sample with a light of a wavelength capable of being absorbed by the FRET donor moiety, followed by waiting an amount of time sufficient for energy transfer from the FRET donor moiety to the FRET acceptor moiety; and
(iii) detecting a light of a wavelength emitted by the FRET acceptor moiety by a fluorescence imaging technique;
wherein the fluorescence intensity detected from the FRET acceptor moiety is indicative of quality and/or quantity of the interaction between the modulator and the protein of interest.

In some embodiments, the present disclosure provides a method of determining an amount of a protein of interest in a sample, the method comprising:
(i) providing the sample comprising the protein of interest attached to a FRET donor moiety and the modulator attached to a FRET acceptor moiety;
(ii) exciting the sample with a light of a wavelength capable of being absorbed by the FRET donor moiety, followed by waiting an amount of time sufficient for energy transfer from the FRET donor moiety to the FRET acceptor moiety; and
(iii) detecting a light of a wavelength emitted by the FRET acceptor moiety by a fluorescence imaging technique;
wherein the fluorescence intensity detected from the FRET acceptor moiety is correlated with the amount of the protein of interest in the sample.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 TR-FRET background. FRET is the non-radiative energy transfer from a donor fluorophore to an acceptor fluorophore that requires (A) close proximity of the fluorophores, and (B) overlap the donor emission spectrum with the acceptor excitation spectrum. (C) TR-FRET utilizes the long excitation lifetime of lanthanides. The time delay between excitation and detection allows for virtual elimination of background signal. (D) The excitation (blue) and emission spectrum (green) of Lumi4Tb and CoraFluors show a large effective Stokes shifts and discrete emission bands

FIG. 2A TR-FRET assay scheme. TR-FRET is installed using various strategies, including (a) antibodies and nanobodies (b) biotin/streptavidin, (c) self-labeling protein tags (e.g. HaloTag), (d) direct chemical labeling (e.g. lysine or cysteine side chains). (e) Representative examples. The POI is tagged using a TR-FRET donor modified antibody. TR-FRET signal is observed upon binding of an acceptor labeled small molecule ligand. Competition with unlabeled ligands causes signal decrease,

FIG. 2B TR-FRET-based ProRS assay design and validation. a, Principle of His6-Halo Tag-ProRS (HT-ProRS) ligand displacement assay. The TR-FRET donor is installed either via labeling of the HaloTag with CoraFluor-1-functionalized Halo Tag ligand (CoraFluor-1-Halo) and/or using a CoraFluor-1-labeled anti-His6 antibody (CoraFluor-1-Pfp). Positive TR-FRET signal is observed upon binding of a suitable tracer labeled with a compatible fluorescence acceptor. Displacement of the fluorescent tracer by a test compound disrupts the signal.

FIG. 2C Principle of TR-FRET-based PRS ligand displacement assays. (A) Structures of MAT334 and TR-FRET tracer MAT379 (FITC shown in green). The FRET donor can be installed via labeling of Halo Tag (B), using a CoraFluor labeled tag-specific antibody (C), or by direct labeling of lysine residues using an amine reactive CoraFluor analog (D). As FRET acceptor for the identification of active site directed inhibitors (B-D)

FIG. 3 Dose-response titration of MAT556 using either CoraFluor-1-Halo-labeled HT-PfcProRS (0.5 nM) or CoraFluor-1-Halo-labeled HT-HsProRS (1 nM), and MAT379 as tracer at 250 nM (for PfcProRS, 2.5×KD and for HsProRS, 0.15×KD) in the absence or presence of 100 uM proline. Data in b are expressed as mean±SD (n≥2 technical replicates) and are representative of 1 independent experiment.

FIG. 4 Synthesis and characterization of MAT574. b-c, Dose-response titration of tracers MAT574, MAT379 and MAT425 using either CoraFluor-1-Pfp-labeled P. aeruginosa ProRS (5 nM) or CoraFluor-1-Pfp-labeled S. aureus ProRS (S nM) in the absence of substrates. d-e, Dose-response titration of proline with CoraFluor-1-Halo-labeled HT-HsProRS (1 nM) and MAT574 as tracer at 50 nM (0.69×KD). Data in b-e are expressed as mean: 1 SD (n≥2 technical replicates) and are representative of 1 independent experiment.

FIG. 5 b-j, Dose-response titration of PheRS tracer MAT588 using various PheRS constructs in the absence of substrates. Plots are labeled according to the method by which the CoraFluor-1 TR-FRET donor was installed (anti-His IgG-Tb is the same antibody setup described in Chapter 2). The CoraFluor-1-Halo (b, e, f, h) and CoraFluor-1-Pfp (c, g, i, j) samples were run with 5 nM of the indicated PheRS. For (d), 2 nM HisHalo-HscPheRS and 4 nM CoraFluor-1-Pfp-labeled anti-His6 antibody were used. The plots in b, c, d, and g have no specific TR-FRET signal which can be explained by either a lack of affinity or too great of separation between the TR-FRET donor (CoraFluor-1) and acceptor (MAT588). Data in b-j are expressed as mean±SD (n≥2 technical replicates) and are representative of 1-2 independent experiments.

FIG. 6 b-c, TR-FRET assay data for serial dilution of HEK293 cell lysates using either GluRS tracer MAT579 at 250 nM (b) or ProRS tracer MAT574 at 250 n.M. (3.5×KD) (c), and a constant CoraFluor-1-labeled anti-EPRS detection mixture consisting of either “5 nM unlabeled ab31531 anti-EPRS polyclonal rabbit IgG antibody+10 nM CoraFluor-1-Pfp-labeled CTK0101 anti-Rabbit-IgG secondary nanobody”, “5 nM CoraFluor-1-Pfp-labeled Proteintech 67712-1-lg anti-EPRS monoclonal mouse IgG2a antibody”, or “5 nM unlabeled CST45956 anti-EPRS polyclonal rabbit IgG antibody+10 nM CoraFluor-1-Pfp-labeled CTK0101 anti-Rabbit-IgG secondary nanobody”. Abcam ab31531 was raised against an Abcam-proprietary, recombinant, full-length HsGluProRS protein. Proteintech 67712-1-1g was raised against a peptide encoding HsGluProRS residues 1163-1512. Cell Signaling Technology CST45956 was raised against a peptide encoding residues surrounding HsGluProRSP978. CTK0101 (Chrom Tek) anti-Rabbit-IgG secondary nanobody (single domain nanobody) was previously labeled with CoraFluor-1-Pfp and validated to bind Rabbit IgG antibodies with high affinity. Data in b-c are expressed as mean±SD (n≥2 technical replicates) and are representative of 1 independent experiment

FIG. 7 ProRS inhibitor design and anti-Plasmodium activity. a. Schematic representation of the ProRS active site and binding mode of canonical substrates (proline, ATP, tRNAPro), halofuginone (1), and T-3767758 (2). The active site of ProRS constitutes three distinct substrate pockets, which bind the terminal adenosine (A76) residue of tRNAPro (red), proline (green), and ATP (yellow). Halofuginone binds in the tRNAPro and proline-binding pockets and requires the presence of ATP for tight binding (ATP-uncompetitive), while compound 2 targets the ATP-binding pocket and requires the presence of proline for tight binding (proline-uncompetitive).

FIG. 8 Comparison of non-conserved ProRS residues adjacent to the active site. a, Comparison of co-crystal structure of T-3767758 (red) and proline (blue) bound to HsProRS (PDB: SVAD) with PfcProRS (PDB: 6T7K). The surface of PfcProRS is shown in light blue and non-conserved residues are highlighted in orange. b, Sequence alignment between HsProRS and PfcProRS. Active site residues are in bold with non-conserved active site or adjacent residues in orange.

FIG. 9 c, Saturation binding of fluorescent tracer MAT379 to CoraFluor-1-labeled HT-PfcProRS (1 nM) in the absence or presence of 100 μM Pro or 500 AM ATP. TR-FRET ratios were background-corrected relative to 10 μM ProSA (˜20,000×KD). d-f, Dose-response titration of reference compounds using CoraFluor-1-labeled HT-PfcProRS (0.25-1 nM) and MAT379 as tracer at 2.5×KD (250 nM) in the absence (d) or presence of 100 μM Pro (e) or 500 μM ATP (f). Under all three conditions, ProSA is titrating HT-PfcProRS. g, Dose-response titration of ProSA using CoraFluor-1-labeled HT-PfcProRS (20 μM), CoraFluor-1-labeled anti-His6 antibody (1 nM), and MAT379 as tracer at 2.5×KD (250 nM). h, Saturation binding of fluorescent tracer MAT379 to CoraFluor-1-labeled HT-HsProRS (1.5 nM). TR-FRET ratios were background corrected relative to 10 μM ProSA (˜20,000×KD). i-j, Dose-response titration of test compounds using CoraFluor-1-labeled HT-HsProRS (1 nM) and MAT379 as tracer at 0.15×KD (250 nM) in the absence (i) or presence of 100 μM Pro (j). ProSA is titrating HT-HsProRS in both conditions and NCP26 is titrating HT-HsProRS in the presence of 100 μM Pro. K, Dose-response titration of test compounds using indicated concentrations CoraFluor-1-labeled HT-HsProRS, 1 nM CoraFluor-1-labeled anti-His6 antibody, and MAT379 as tracer at 0.15×KD (250 nM).

FIG. 10 Additional characterization of TR-FRET tracers MAT379 (24) and MAT425. a-b, Saturation binding of fluorescent tracer MAT425 to (a) CoraFluor-1-labeled HT-PfcProRS (1 nM) or (b) CoraFluor-1-labeled HT-HsProRS (1.5 nM). TR-FRET ratios were normalized relative to 10 μM ProSA (˜20,000×KD). Data in a and b are shown as mean±s.d. (n=3 technical replicates) and are representative of at least 2 independent experiments. c, Determination of dissociation kinetics for tracer MAT379. An equilibrated solution of 100 nM CoraFluor-1-labeled HT-PfcProRS (circles) or HT-HsProRS (squares) and ˜EC80 MAT379 (560 nM for HT-PfcProRS and 7 AM for HT-HsProRS) was diluted 10-fold into assay buffer containing no ProRS or MAT379 and the TR-FRET ratio (520/490 nm) was measured in ˜45 s intervals over the course of 10 min. TR-FRET ratios were normalized relative to both the initial time point and 10 μM ProSA (˜20,000×KD) prior to dilution. Kinetics data in c are expressed as mean #s.d. (n=23 technical replicates) and are representative of >2 independent experiments. d, Summary of binding kinetics data for MAT379 determined using the TR-FRET-based ligand displacement assay. The dissociation rates (koff) were experimentally determined, but the association rates (kon) were too fast to measure (kon,obs) so they were instead calculated (kon,calc) using the corresponding equilibrium dissociation constant (KD) and koff value

FIG. 11 Asexual blood stage P. falciparum activity of ATP-site targeted pyrazinamide-derived ProRS inhibitors. a-c, In vitro characterization of pyrazinamides 4 (a), 6 (b), and 7 (c) in wildtype (Dd2-2D4; circles and solid lines), halofuginone-induced (squares and dashed lines), and HFGR-I (triangles and dotted lines) ABS P. falciparum parasites. d-g, In vitro characterization of ProRS inhibitors in Dd2-2D4 wildtype (d and g), halofuginone-induced (e), and HFGR-I (f) ABS P. falciparum parasites. Data in panels d and g was split for visualization purposes only. Data are expressed as mean±s.d. (n=3 technical replicates) and are representative of at least 3 independent experiments, except for 8, 13, and 14 which had one bioreplicate each in Dd2-2D4.

FIG. 12 ProRS Substrate Affinity Determination. Dose-response titration of proline (blue) or ATP (red) using CoraFluor-1-labeled HT-PfcProRS (5 nM, circles) or CoraFluor-1-labeled HT-HsProRS (1.5 nM, squares) and tracer MAT379 (50 nM for HT-PfcProRS and 1,000 nM for HT-HsProRS). TR-FRET ratios were background-corrected relative to 10 p M ProSA (˜20,000×KD). Data are expressed as mean±s.d. (n=4 technical replicates) and are representative of >>2 independent experiments

FIG. 13A-D structures of exemplified compounds.

FIG. 14 Overlay of all TR-FRET-based ProRS assay data. Dose-response titration of ProRS inhibitors in the absence (a, d) or presence of 100 μM Pro (b, c) or 500 μM ATP (c, f) using CoraFluor-1-labeled HT-PfcProRS (0.020-1 nM) or HT-HsProRS (0.050-1.5 nM) and MAT379 as tracer at 250 nM (2.5×KD for HT-PfcProRS and 0.15×KD for HT-HsProRS). Compounds marked with * were supplemented with 1 nM CoraFluor-1-labeled anti-His6 antibody. Compounds marked with ** were titrating ProRS under these conditions. Data are expressed as mean±s.d. (n≥2 technical replicates) and are representative of ≥2 independent experiments.

FIG. 15 Correlation between TR-FRET pKD values and P. falciparum asexual blood stage growth assay pEC50 values. a, Comparison of TR-FRET pKD values for HT-HsProRS (x-axis) vs HT-PfcProRS (y-axis). b, Comparison of asexual blood stage (ABS) P. falciparum Dd2-2D4 pEC50 (x-axis) vs HT-PfcProRS TR-FRET pKD value (y-axis). Data are expressed as the respective mean values and are representative of ≥2 independent experiments. TR-FRET pKD values shown are from the highest affinity conditions (i.e. data from absence of substrates for ATP- and proline-competitive inhibitors, 100 μM Pro for proline-uncompetitive inhibitors, and 500 μM ATP for ATP-uncompetitive inhibitors). Spearman correlation coefficients (rs) for both plots are calculated using only pyrazinamide compounds (i.e. excluding ProSA, D-ProSA, halofuginone, halofuginol, and glyburide).

FIG. 16 NCP26-resistance selection and whole genome sequence analysis. a. In vitro activity of NCP26 against ABS P. falciparum Dd2-2D4 (parent) or subclones from three independent resistance selection experiments (S1-3). SI did not yield resistant parasites and S1-clone A8 was included for comparison. b, In vitro activity of reference compounds against Dd2-2D4 (parent) or subclones from each selection (S1-3). Reference compounds include PfcProRS inhibitors (halofuginone and halofuginol) and non-PfcProRS inhibitors dihydroartemisinin (DHA) and borrelidin (P. falciparum threonyl-tRNA synthetase inhibitor). c, Residues T512S (S2) and F405L (S3) were identified by whole genome sequence analysis to mediate NCP26-resistance and are mapped to the co-crystal structure of NCP26 (green) and proline (white) bound to wildtype PfcProRS (PDB: 6T7K). Both amino acid side chains directly interact with the pyrazinamide core. d, Whole genome sequence analysis revealed amplification of the intra-chromosomal region harboring the PfcProRS locus on chromosome 12 that is observed in all S3 clones. No copy number variations were observed in other chromosomes for S3 clones or in any chromosomes for Dd2-2D4 (parent), SI (no NCP26-resistance observed), or S2 (PfcProRST512S).

FIG. 17. a. Chemical structures of pyrazinamide-proline hybrids (absolute stercochemistry). b, Overlay of the co-crystal structures of PfcProRS (grey surface) in complex with proline (orange sticks) and either NCP26 (PDB: 6T7K, yellow sticks), MAT334 (29) (PDB: 7QC2, green sticks), and MAT345 (30) (PDB: 7QB7, pink sticks) shows the prolyl-substituents of MAT334 and MAT345 pointing outside the active site. c-d, Dose-response titration of pyrazinamide-proline hybrids using CoraFluor-1-labeled HT-PfcProRS (0.25-1 nM) and MAT379 as tracer at 2.5×KD (250 nM) in the absence (c) or presence of 100 AM Pro (d). See Supplementary FIG. 8 for corresponding data with HT-HsProRS in the presence or absence of 100 AM Pro. e, In vitro activity of pyrazinamide-proline hybrids against wild-type (Dd2-2D4), balofuginone-induced (HFG-induced), and HFGR-I P. falciparum asexual blood stage. TR-FRET assay data in c and d are expressed as mean±s.d. (n≥2 technical replicates) and are representative of at least 2 independent experiments. ABS growth assay data in e are expressed as mean±s.d. (n=3 technical replicates) and are representative of at least 3 independent experiments, except for compound 31 which was tested once for each strain shown.

FIG. 18 Data collection and refinement statistics for PfcProRS ligand complexes. Data were collected from one crystal for each structure. Statistics for the highest-resolution shell are shown in parentheses.

FIG. 19 Characterization of dual-site ligands binding HsProRS in the absence or presence of proline. Dose-response titration of pyrazinamide-proline hybrids in the (a) absence or (b) presence of 100 μM Pro using CoraFluor-1-labeled HT-HsProRS (1.5 nM) and MAT379 as tracer at 0.15×KD (250 nM). Data are expressed as mean±s.d. (n≥2 technical replicates) and are representative of ≥2 independent experiments.

FIG. 20 Structural comparison of free and ligand-bound HsProRS. a, Overlay of HsProRS crystal structures in the apo state (green, PDB: 4K86), bound to proline alone (yellow and pink, respectively; PDB: 7OSY), and bound to both proline and 2 (blue, orange, and white, respectively; PDB: 5VAD) reveals significant allosteric structural changes upon proline binding, including the ATP-binding pocket and the active site entry. Selected residues in and adjacent to the active site are shown as lines. b, Apo crystal structure of HsProRS (PDB: 4K86). c, Co-crystal structure of HsProRS (yellow) bound to proline (pink, PDB: 70SY). d, Co-crystal structure of HsProRS (blue) bound to proline (orange) and 2 (white, PDB: 5VAD).

FIG. 21 Conventional assay platforms for measuring protein levels and target engagement. (A) Western blot, where proteins are separated by SDS-polyacrylamide gel electrophoresis (SDS-PAGE), transferred to nitrocellulose membranes, and are detected with a primary antibody/HRP-linked secondary system with photodetection as the readout. (B) Sandwich enzyme-linked immunosorbent assay (ELISA). An immobilized capture antibody first binds the POI. After, an enzyme-conjugated detection antibody is added and protein is detected via colorimetric readout. (C) Homogenous time-resolved fluorescence (HTRF) immunoassay. Similar to a sandwich ELISA, orthogonal antibody pairs are used. However, in HTRF antibodies are labeled with a TR-FRET donor and acceptor; concomitant binding to the POI results in an increase in TR-FRET signal. (D) TR-FRET ligand displacement assay. Recombinant, epitope-tagged proteins are incubated with a TR-FRET donor-labeled anti-epitope tag antibody and a fluorescent tracer. Subsequent addition of test compounds displace the fluorescent tracer, resulting in a decrease in TR-FRET signal. Both Western blot and sandwich ELISA-based approaches are generally low-throughput, while ELISA assays are generally more quantitative in nature. In contrast, HTRF-based immunoassays are both quantitative and higher in throughput, yet, like sandwich ELISAs, require matched antibody pairs which are often difficult to obtain

FIG. 22A single-antibody TR-FRET platform to quantitatively measure small molecule target engagement and endogenous protein levels in whole cell extracts. (A) Quantification of both small molecule target engagement and protein levels with endogenous protein targets, here for BRD4. The detection mix consists of a single primary antibody, CoraFluor-1-labeled nano-secondary, and a fluorescent JQ1-based tracer. (B) Chemical structures of bromodomain inhibitors, degraders, and tracers used in this study. (C) TR-FRET-based BRD4 quantification (see STAR Methods) in serially diluted MCF7 cell lysate shows linearity over approximately three orders of magnitude (n=2). (D-F) Dose-titration of small molecule inhibitors and degraders in TR-FRET ligand displacement assays with (D-E) recombinant BRD4 (BD1) and BRD4 (BD2) domains, and (F) endogenous BRD4 in MCF7 cell extract (0.8 mg/mL total protein) (n=2). Data were fitted to a four-parameter dose-response model in Prism 9. Data in (C-F) are expressed as mean±SD of n technical replicates and are representative of at least two independent experiments.

FIG. 23. Determination of apparent equilibrium dissociation constant for JQ1-FITC to recombinant bromodomains and endogenous BRD4. Conditions include (A) 0.5 nM GST-BRD4 (BD1), 2 nM CoraFluor-1-labeled anti-GST VHH, (B) 0.5 nM GST-BRD4 (BD2), 2 nM CoraFluor-1-labeled anti-GST VHH, (C) 0.8 mg/mL total protein MCF7 lysate, 0.5 nM rabbit anti-BRD4 IgG, 1 nM CoraFluor-1-labeled anti-rabbit nano-secondary (endogenous BRD4). Measured KD,app values and associated 95% confidence intervals (shown in parentheses) are displayed in panel (D). Data in (A-C) are expressed as mean±SD of n=3 technical replicates and are representative of at least two independent experiments. Data were fitted to a one-site model using Prism 9.

FIG. 24 contains a table showing apparent equilibrium dissociation constants for individual recombinant bromodomains and endogenous BRD4 determined by biochemical TR-FRET ligand displacement assays.

FIG. 25 contains a table showing cellular degradation constants for small molecule BRD4 degraders determined by TR-FRET.

FIG. 26 contains a table showing apparent equilibrium dissociation constants for CS and CS-JQ1 toward Keap1-Kelch and Keap1-BTB domains.

FIG. 27 TR-FRET-based quantification of BRD4 levels in unmodified cell lines after degrader treatment. BRD4 protein levels in cell lysate after 5 h treatment with dBET6 (positive control) and JQ1 (negative control) were measured with TR-FRET assay as described in FIG. 1A. Assays were run in a 24-well plate format with either (A) MCF7 or (B) MDA-MB-231 cells. Cells were lysed and BRD4 was quantified via addition of TR-FRET detection mix (see STAR Methods). The total time between cell treatment and TR-FRET measurement was ˜1.5 h. dBET6 showed potent degradation (DC50, 5 h,MCF7=8.1±1.5 nM, Emax,5h,MCF7=1.1%; DC50, 5 h, MDA-MB-231=4.1±0.3 nM; Emax,5h,MDA-MB-231=1.2%) while JQ1 did not induce BRD4 degradation. Western blot analysis on the same dBET6-treated samples are shown in the bottom panel and are in good agreement with TR-FRET quantification (n=2). (C-D) Quantitative profiling of BRD4 degradation rescue by co-treatment of (C) MCF7 or (D) MDA-MB-231 cells with 1 μM BTZ (20S proteasome), MLN7243 (E1 ubiquitin-activating enzyme), MLN4924 (NEDD8) or 10 μM JQ1 (competing ligand) and 250 nM dBET6 after 5 h shows efficient attenuation of degradation (n=2). Data in (A-D) are expressed as mean±SD of n biological replicates.

FIG. 28 Assay miniaturization and assessment of robustness in 96-well plate format. (A) MDA-MB-231 cells (20,000 cells/well) in 96-well plates were treated with a dose-titration of dBET6 or JQ1 for 5 h. BRD4 levels were quantified via subsequent addition of lysis buffer (60 μL) and detection mix (10 μL) followed by TR-FRET signal acquisition after 1 h incubation (see STAR Methods). dBET6, but not JQ1, induced potent BRD4 degradation (DC50, 5 h=3.2+0.1 nM, R2=0.99, Emax,5h=0.6%). Data are expressed as mean±SD of n=3 technical replicates. Data were fitted to a four-parameter dose-response model in Prism 9. (B) Z′-factor measurement for TR-FRET quantification assay in 96-well plate format with MDA-MB-231 cells. The Z′-factor was calculated from n=24 positive control wells (DMSO-treated cell lysate) and n=24 negative control wells (DMSO-treated cell lysate+50 μM JQ1-Acid to simulate 100% BRD4 degradation), with or without CellTiter-Glo 2.0 normalization (also see FIG. 29). Data in (A-B) are representative of two independent experiments.

FIG. 29 Z′-factor measurement for CellTiter-Glo 2.0. CellTiter-Glo 2.0 reagent (5 μL) was added to either MCF7 cell lysate (0.5 mg/mL; 30 μL; n=96 positive control wells) or lysis buffer containing no cell extract (30 μL; n=12 negative control wells) in a white, 384-well microtiter plate (Corning 3572) and allowed to equilibrate for 10 min at room temperature, after which point luminescence signal was read on a Tecan SPARK plate reader. The Z′-factor is a statistical measure of assay quality using control data, in this case the negative control being lysis buffer in the absence of cell extract (no cellular ATP) and was found to be 0.83, indicating an excellent assay. Data are representative of two independent experiments.

FIG. 30 contains chemical structures of CDDO and CDDO-Me. Thiophilic site is shown as a grey circle.

FIG. 31A shows that celastrol is a powerful E3 ubiquitin ligase recruiter for targeted protein degradation applications. The figure shows chemical structures of celastrol (CS) and celastrol-JQ1 (CS-JQ1; 2). Thiophilic sites are highlighted with grey circles.

FIG. 31B shows quantification of target engagement of CS-JQ1 with recombinant BRD4 (BD1), BRD4 (BD2), and endogenous BRD4 in MCF7 cell extracts (n=2). Data were fitted to a four-parameter dose-response model in Prism 9 (n=2, Kelch; n=4, BTB).

FIG. 31C shows dose-titration of CS and CS-JQ1 in TR-FRET assays with full-length Keap1 (see STAR Methods). Data were fitted to a four-parameter dose-response model in Prism 9 (n=2, Kelch; n=4, BTB).

FIG. 31D shows CS-JQ1-induced ternary complex formation between full-length Keap1 and BRD4 (BD1) and BRD4 (BD2) (n=2).

FIG. 31E shows TR-FRET quantification of BRD4 levels in MCF7 and MDA-MB-231 cells after treatment with dose-titrations of CS-JQ1 for 5 h in 24-well plate assay format. The DC50, 5 h and Emax, 5 h values for CS-JQ1 were 29+14 (21%) and 16 #2 nM (12%) in MCF7 and MDA-MB-231 cells, respectively (n=2).

FIG. 31F shows western blot analysis of the same samples used for TR-FRET quantification in FIG. 1E.

FIG. 31G shows western blot analysis of the same samples used for TR-FRET quantification in FIG. 1E.

FIG. 31H shows rescue of CS-JQ1-induced BRD4 degradation (250 nM) by BTZ, MLN7243, MLN4924 (1 AM) and JQ1 (10 μM) in MCF7 cells (n=2).

FIG. 31I shows rescue of CS-JQ1-induced BRD4 degradation (250 nM) by BTZ, MLN7243, MLN4924 (1 AM) and JQ1 (10 μM) in MDA-MB-231 cells (n=2).

FIG. 31J shows that co-treatment of MDA-MB-231 cells with CS-JQ1 (250 nM) and potent BTB-targeting Keap1 ligand CDDO-Me (1 μM) does not attenuate BRD4 degradation, indicating potential activity mediated through additional E3 ligase complexes other than Keap1/CRL3 (n=2). Data in FIG. 1B-FIG. 1D are expressed as mean±SD of n technical replicates and are representative of at least two independent experiments. Data in FIG. 1E-FIG. 1J are expressed as mean±SD of n biological replicates.

FIG. 32A shows HSFP6×His expression and Ni-NTA purification.

FIG. 32B shows labeling of HSFP6×His by Cora-1-Halo and Cora-1-SNAP.

FIG. 32C shows labeling of EGFP-Halo Tagin live cells with Cora-2-Halo.

FIG. 33. Chemical structures, photophysical and physicochemical characterization of representative CoraFluors. Chemical structures of (a) Lumi4TM ligand (Cisbio, PerkinElmer), (b) carboxylinker-modified ligands (1-3) and CoraFluors (4-6), (c) CoraFluors functionalized as Halo Tag, SNAP-tag, and active ester derivatives, (d) linker-less core complexes 12-14. (c) Absorption and emission spectra for Cora-1-Halo (blue), Cora-2-Halo (orange), Cora-3-Halo (red) shown in comparison to 12 (black); absorbance maxima of halogenated CoraFluor analogs Cora-2-Halo and Cora-3-Halo are redshifted (λmax=356 nm) compared to Cora-1-Halo and 12 (λmax=340 nm). The emission spectra (solid green) are virtually identical for all complexes. (f) Luminescence lifetime: all complexes display exponential decay with varying luminescence lifetimes (n=50). (g) Excitation/emission kinetics: complexes exhibit characteristically slow excitation rates when using LED excitation, with halogenated complexes displaying faster excitation kinetics and/or time to reach steady-state equilibrium (n=10). (h) Kinetic stability of Cora-1/2/3-Halo and 12 (5 nM) toward 1 mM EDTA in Tris, pH 7.5. (i) Stability profiling of Cora-1/2/3-Halo and 12 (5 nM) after 7 d exposure to various buffers (50 mM), additives (DTPA, Ca2+, Mg2+, DTT, TCEP=1 mM; Mn2+, Cu2+=0.5 mM), and pH ranges. All stability experiments were performed at room temperature. Unless noted otherwise, all data are expressed as the mean±SD (n=3).

FIG. 34. Biochemical validation of CoraFluors with HSFP6×His test system. (a) Schematic of HaloTag-SNAP-tag-6×His (HSFP6×His) fusion construct used in biochemical assays for validation of CoraFluors and respective labeled protein conjugates. (b) Detection of specific, TEV protease-cleavable TR-FRET signal between Cora-1-SNAP and both FITC- and TMR-Halo ligands using HSFP6×His platform. (c) Validation of Cora-1-Pfp labeled anti-6×His antibody (Abcam, 18184), displaying specific TR-FRET signal between the donor-labeled conjugate and FITC- and TMR-Halo ligands. (d) Limit of detection of donor emission (blue) and Tb/FITC TR-FRET signal (520/490 nm; orange) for a dose-titration of HSFP6×His conjugate labeled with Cora-1-SNAP and FITC-Halo, showing accurate quantification of TR-FRET signal at femtomolar levels using a monochromator-based plate reader (Tecan SPARK). (c) Saturation binding of HSFP6×His-FITC-Halo to IgG monoclonal anti-6×His CoraFluor-1 conjugates (0.5 nM) reveals significantly differential binding affinities for two commercial antibody clones (Abcam, 18184 and BioXCell, RT0266). (f) Saturation binding of FITC-Halo or Cora-1-SNAP labeled HSFP6×His to CoraFluor-1- or AF488-labeled anti-HaloTag nanobodies (0.5 nM; Chromo Tek, Halo Trap), respectively. No significant change of binding affinity was observed (n=2). Unless noted otherwise, all data are expressed as the mean±SD (n=3).

FIG. 35 CoraFluors enable versatile and domain-specific interrogation of Keap1-Keap1 and Keap1-small molecule interactions. (a) Schematic representation of Keap1/CRL3 complex. Keap1 forms a homodimer through its BTB domain and binds to Cul3. Nrf2 is recruited via binding to both Kelch domains. (b) Depiction of multimodal TR-FRET assay design principle organized around differentially donor- and acceptor-labeled Keap1 ligands and/or with labeled anti-epitope tag antibodies. (c) Chemical structures of Keap1 tracers and small molecule inhibitors used in this study. Thiophilic sites are represented by orange dots. (d) Off-rate measurements (koff) for FITC-KL9 (black) and CDDO-FITC (grey) tracers. (e) Keap1-Keap1 dimer off-rate (k_{off,dimer}) measurement, determined by rapid dilution of FITC-KL9/Cora-1-KL9-saturated homodimer into buffer containing isomolar FITC-KL9/Cora-1-KL9 concentrations. (f) Saturation binding experiment to determine Keap1 dimer equilibrium dissociation constant. K_{d,dimer} was determined by linear regression extrapolation of background-corrected, log-transformed equilibrium TR-FRET ratios (solid red circles). Open red circles and open black triangles represent non-corrected, log-transformed and background-corrected, linear-plotted TR-FRET ratios, respectively (n=4). (g) Dose-response titration of inhibitor set in Assay-1 with FITC-KL9/Cora-1-KL9 as tracers using untagged, full-length, wildtype Keap1, with Kelch binders displaying predicted potency and thiophilic inhibitors showing lower albeit significant binding at higher concentration. (b) In Assay-2, using Cora-1-KL9 and CDDO-FITC to complete the FRET pair, Kelch binders retain activity and the potency of thiophiles is significantly shifted, indicating more potent binding to Cys151. Highly differential activity (>250-fold) was observed between CDDO/CDDO-Me and CDDO-JQ1. For g and h, tabulated Kᵢ values (uM) of Keap1 inhibitors as determined in Assay-1 and Assay-2 (also see tables herein). Unless noted otherwise, all data are expressed as the mean±SD (n=3).

FIG. 36 CoraFluor performance in TR-FRET ligand-displacement assays for HDAC1-HaloTag in crude lysate from transiently transfected HEK293T cells. (a) Structures of HDAC tracers (M344-FITC, SAHA-NCT) and representative HDAC inhibitors (SAHA, panobinostat, CI-994, Cpd-60) used in this study. (b) Spectral overlap of Tb emission (black, solid) with M344-FITC (orange, dashed) and SAHA-NCT (blue, dashed) absorbance, alongside M344-FITC (orange, solid) and SAHA-NCT (blue, solid) emission. (c) Schematic showing the TR-FRET assay principle wherein covalently bound CoraFluor Halo Tag-ligands produce specific TR-FRET signal with fluorescent HDAC tracers M344-FITC and SAHA-NCT, which can be competed by unlabeled HDAC inhibitors. Here, the specific nature of the Halo Tag renders this assay format compatible with cell lysates, where binding to HDAC1 can be measured in the direct presence of other HDAC isoforms. (d) Saturation binding of M344-FITC (orange) and SAHA-NCT (blue) to Cora-1-Halo labeled HDAC1-HT in overexpression lysate. (c) Dose-response titration of test compounds in HEK293T expression lysate using M344-FITC (open shapes) or SAHA-NCT (solid shapes) as tracers at concentrations near their respective K_{d} values. Kᵢ-values obtained with both tracers are virtually identical and match inhibitory constants determined by other assay platforms (see Supplementary Table 2). (f) Kinetic profiling of HDAC1 inhibition by Cpd-60 (tracer: M344-FITC), confirms time-dependent activity and slow-binding kinetics, resulting in >100-fold differential apparent Kᵢ over the course of 24 h. Unless noted otherwise, all data are expressed as the mean±SD (n=3).

FIG. 37 CoraFluors enable TR-FRET based target engagement profiling in live cells. (a) Overnight treatment of HEK293T cells transiently transfected with HDAC1-HT in medium supplemented with as low as 12.5 AM Cora-2-Halo labeled >70% of HDAC1-HT protein relative to DMSO control. (b) Optimization of SAHA-NCT tracer concentration in live-cell TR-FRET target engagement assays with Cora-2-Halo labeled HEK293T cells expressing HDAC1-HT. Labeled cells were treated with varying doses of SAHA-NCT (blue) or M344-FITC (orange; cell-impermeable negative control) in the presence or absence of 25 μM panobinostat. In live cells (solid shapes), specific TR-FRET signal was detected with cell-permeable SAHA-NCT, but not with cell-impermeable M344-FITC. Upon addition of lysis buffer and permeabilization of cellular membranes, both tracers displayed specific TR-FRET signal (open shapes) (n=6). (c) Cellular dose-dependent inhibition of Cora-2-Halo labeled HDAC1-HT in HEK293T cells by various HDAC inhibitors (treatment 4 h at 37° C., 1 μM SAHA-NCT, n=6). (d) Tabulated EC₅₀ and apparent Kᵢ (K_{i,app}) values (μM) determined (via Cheng-Prusoff) in cellular target engagement assay. ^{a}Values from (52); ^{b}Biochemical IC₅₀; NA, not available. Unless noted otherwise, all data are expressed as the mean±SD (n=3).

FIG. 38A Existing synthetic strategy for Lumi4TM ligand according to patent WO2008063721A3 (64).

FIG. 38B Time-dependent stability profiling: the stability of terbium complexes (5 nM) to standard buffers (50 mM) and pH ranges at room temperature was monitored over seven days. Data are represented as means±SD of three replicates (n=3).

FIG. 38C Time-dependent stability profiling: the stability of terbium complexes (5 nM) to reducing agents (dithiothreitol, DTT, tris (2-carboxyethyl) phosphine, TCEP) at room temperature (pH 7.5) was monitored over seven days. Data are represented as means±SD of three replicates (n=3).

FIG. 38D Time-dependent stability profiling: the stability of terbium complexes (5 nM) to bivalent metal ions (Mg²⁺, Ca²⁺, Cu²⁺, Mn²⁺) at room temperature (pH 7.5) was monitored over seven days. Data are represented as means±SD of three replicates (n=3).

FIG. 38E Time-dependent stability profiling: the stability of terbium complexes (5 nM) to various concentrations of ethylenediaminetetraacetic acid (EDTA; 0.1, 1, 25 mM) at room temperature (pH 7.5) was monitored over seven days. Data are represented as means±SD of three replicates (n=3).

FIG. 38F Time-dependent stability profiling: the stability of terbium complexes (5 nM) to various concentrations of diethylenetriaminepentaacetic acid (DTPA; 0.1, 1, 25 mM) at room temperature (pH 7.5) was monitored over seven days. Data are represented as means±SD of three replicates (n=3).

FIG. 38G Cora-1-Halo and Cora-1-SNAP are efficient substrates for their self-labeling protein tags (HaloTag, SNAP-tag, respectively). The competition of TMR-Halo and TMR-SNAP labeling of HSFP6×His construct via the respective CoraFluor complexes (2 h incubation) was assessed by SDS-PAGE and Cy3 fluorescence gel imaging (Typhoon FLA 9500). The CoraFluor complexes are not fluorescent under Cy3 fluorescence gel imaging.

FIG. 38H Chemical structures of Halo Tag and SNAP-tag ligands used in this study.

FIG. 38I Qualitative monitoring of HDAC1-Halo Tag expression, localization and transfection efficiency via fluorescence imaging with TMR-Halo. HEK293T cells were seeded into 24-well plates (Corning) at 50,000 cells/well in phenol red-free culture media and allowed to recover for 24 h. Cells were then transfected with pFC14A-HDAC1-Halo Tag/PEI cocktail (see Methods) and grown for an additional 24 h before the addition of 100 nM TMR-Halo to the media. Cells were treated for 1 h at 37° C. and 5% CO₂ and imaged at 5×objective (5x/0.17 Plan-NEOFLUAR) on a Zeiss Axio Observer fluorescent microscope equipped with Zeiss filter set 45 (BP 560/40, FT 585, BP 630/75) and EXFO X-Cite 120 XL excitation light source.

FIG. 38J Stability of 10 μM Cora-2-Halo over 24 h in phenol red-free cell culture medium (DMEM+10% FBS+1% P/S) at 37° C. and 5% CO₂. Data are represented as means±SD of ten replicates (n=10).

FIG. 38K Inhibition of HaloTag labeling by Roche Complete™ Protease Inhibitor Cocktail tablets and an unknown component(s) of LB-Miller broth. Purified HSFP6×His conjugates labeled with either Cora-1-SNAP alone or Cora-1-SNAP/Ac-Halo (negative control) were diluted into different buffers to 25 nM then FITC-Halo was dose-titrated from 0 to 200 nM (2 h incubation). Corrected TR-FRET ratios were obtained via subtraction of the pre-blocked (Ac-Halo) negative control on a per-buffer basis. Buffer recipes are as follows: PBS, 50 mM sodium phosphate, 150 mM NaCl, 0.05% (v/v) TWEEN-20, pH 7.5; Lysis buffer 1, 50 mM Tris, 150 mM NaCl, 2 mM DTT, 1% (v/v) Triton X-100, 0.1% (w/v) sodium deoxycholate, pH 7.5; Lysis buffer 2, 50 mM Tris, 150 mM NaCl, 0.8% (v/v) IGEPAL-CA630, 5% glycerol, 1.5 mM MgCl₂, 2 mM DTT, pH 7.5; HDAC buffer, 50 mM HEPES, 100 mM KCl, 0.5 mg/mL BSA, 0.001% (v/v) Tween-20, pH 7.5; LB Broth, Luria-Bertani broth (MilliporeSigma 71-753-5).

FIG. 39A Three-dimensional representation of CoraFluor complex. Model of macrotricyclic terbium complex with tertiary amide linker attachment (upper left). The model was generated in Chem-3D (ChemDraw, PerkinElmer, Waltham, MA). The terbium center is shown as a green sphere.

FIG. 39B Synthetic scheme to access CoraFluor ligands. Reagents and conditions: (a) TsCl, K₂CO₃, H₂O, rt, 48 h; (b) NaOH, H₂O, 0° C., 2 h (43% over 2 steps); (c) ethylenediamine, 10 mol % p-TsOH, MePh, 60° C., 24 h (92%); (d) HBr, AcOH, 115° C., 24 h (>95%); (e) ethyl 6-bromohexanoate, K₂CO₃, ACN, 80° C., 12 h then KOH, H₂O, 95° C., 2 h; (f) HBr, AcOH, 115° C., 24 h then EtOH, HBr (cat.), 85° C., 2 h (54% over 2 steps); (g) 46, 47, or 48, DIPEA, DMF, rt, 12 h (>95%); (h) 40, PyBOP, DIPEA, DMF, rt, 1-3 h, 2-5 mM (40-70%); (1) HBr, AcOH, 100° C., 30 min then NaOH, H₂O, rt, 10 min then aqueous HBr (>95%); (/) isobutyl chloroformate, DIPEA, DCM, rt, 10 min then tetrafluorophenol, DMAP (cat.), rt, 12 h (60-80%).

FIG. 40. CoraFluor-2 exhibits improved excitability at 405 nm. (a) Visual comparison of luminescence intensities of CoraFluors under constant illumination with a 365 nm LED (left image) or a 405 nm laser diode (right image) demonstrates significantly enhanced luminescence intensity of Cora-2-Halo compared to Cora-1-Halo with 405 nm but not 365 nm excitation. Excitation light is passed through the adjacent samples from the left, eliminating potential light filtering effects from Cora-2-Halo, which exhibits a higher molar absorptivity at the tested wavelengths. (10 AM CoraFluor in 50 mM HEPES buffer, pH 7.4). (b) Comparative quantitative analysis of excitation wavelength-dependent, time-resolved luminescence intensity of CoraFluors demonstrates that Cora-2-Halo offers superior signal intensity following 405 nm excitation (200 nM CoraFluors in 50 mM HEPES buffer, pH 7.4, constant photomultiplier gain, acquisition delay=100 μs, and integration time=50 μs). Excitation wavelength (bandwidth=5 nm) was varied in 5 nm increments, and the TR-fluorescence response of Cora-1-Halo and Cora-2-Halo was measured relative to background (buffer alone). Data were background-corrected, normalized and are represented as mean±SD of 16 replicates. Data were acquired on a Tecan SPARK plate reader in a white 384-well plate (Corning 3572).

FIG. 41 Select photophysical characterization data for CoraFluors and linker-less complexes. (a) Quantum yield plots for select terbium complexes. (b) Background-corrected decay curves and calculated luminescence lifetimes for linker-less (12-14) and select CoraFluor complexes. Luminescence intensity values were normalized, In-transformed and linear regression analysis was performed in Prism 8. Data are represented as means±SD of fifty replicates (n=50).

FIG. 42 Characterization of Keap1 fluorescent tracers and their use in single-ligand displacement TR-FRET assays. (a-e) Saturation binding of (a) FITC-KL9 against Keap1 (His/GST) construct (1 nM) with 0.5 nM Tb-Anti-6×His, (b) Cora-1-KL9 against Keap1 (His/GST) construct (1 nM) with 0.5 nM AF488-Anti-6×His, (c) FITC/Cora-1-KL9 mixture against Keap1 (tag-free) construct (1 nM), (d) CDDO-FITC against Keap1 (His/GST) construct (1 nM) with 0.5 nM Tb-Anti-6×His, and (e) CDDO-FITC against Keap1 (tag-free) construct (5 nM) with 5 nM Cora-1-KL9. The equilibration dissociation constants (K_{d} and K_{d,app}) were calculated in Prism 8 (GraphPad Software) using a one-site-binding (a-d) or four-parameter (e) nonlinear regression fit model. (f-g) Dose-response curves for Keap1 inhibitor test set as measured in TR-FRET assays with recombinant, full-length Keap1 with N-terminal 6×His/GST tags and FITC-KL9 tracer (f) or CDDO-FITC tracer (g). Conditions: 1 nM Keap1 (His/GST) construct, 0.5 nM Tb-Anti-6×His, and either (f) 5 nM FITC-KL9 or (g) 40 nM CDDO-FITC, 4 h incubations. See Table herein for measured IC₅₀ values. In these dose-response assays, due to higher-order oligomeric complex formation, we did not attempt to determine true K_{d} values of inhibitors from the measured IC₅₀ values. However, relative potencies between the inhibitors profiled remained constant. Data are represented as means±SD of three replicates (n=3).

FIG. 43 Cell permeability profiling of select CoraFluors with EGFP-HaloTag expression construct. (a) Labeling of intracellular EGFP-Halo Tag construct in HEK293T cells by Cora-2-Halo, but not Cora-1-Halo, in a dose-dependent manner. Cells were treated with the indicated concentrations of Halo Tag-ligands (or DMSO) in phenol red-free Opti-MEM for 4 h at 37° C. before being washed, lysed in the presence of 10 μM TMR-Halo, and assessed for competition of TMR-Halo labeling via SDS-PAGE. (b) Detection of specific TR-FRET signal between EGFP-Halo Tag and Cora-2-Halo in live cells after treatment with 50 AM Cora-2-Halo for 4 h at 37° C. Data are represented as means±SD of sixteen replicates (n=16).

FIG. 44 Mammalian expression and lysate-based quantification of HDAC1-HaloTag construct. (a) Expression, Cora-1-Halo labeling, and TR-fluorescence-based quantification of HDAC1-HaloTag construct in HEK293T overexpression lysate. After incubation with 10 μM Cora-1-Halo, the lysate is gel filtrated to remove excess HaloTag ligand. Because labeling is stoichiometric (1:1 Cora-1-Halo: HDAC1-HaloTag), the concentration of Cora-1-Halo labeled HDAC1-HaloTag in the lysate can accurately be determined via a reference calibration curve (here measuring Tb emission at 548 nm, 340/50 excitation, 100 μs delay, 400 μs integration, 0-230 nM and 10 nM increment calibration curve). In our experience, the yield of HDAC1-Halo Tag from a single 15 cm dish of transfected HEK293T cells (˜25 million cells) was between 25-50 μg, giving protein concentrations between 200-500 nM and, therefore, samples were diluted ˜1:5 to remain within the standard curve (green square). (b) Quantification of HDAC1-HaloTag (Cora-1-Halo labeled) in HEK293T cell overexpression lysate with AF488-HaloTrap. The labeled lysate was diluted 1:12 (275 μg/mL total protein) and incubated with varying concentrations of Halo Trap-AF488 (0-150 nM, 16-point). The concentration of Cora-1-Halo labeled HDAC1-HaloTag in the diluted lysate was determined by nonlinear regression analysis following a quadratic equilibrium-binding equation (see Methods). Data in (a) and (b) are represented as means±SD of three replicates (n=3) and two replicates (n=2), respectively.

FIG. 45 Biochemical validation of HDAC fluorescent tracers and inhibitors with purified, recombinant protein. (a) Saturation binding curves for fluorescent HDAC tracers (SAHA-NCT, M344-FITC) using recombinant HDAC1. Conditions: 5 nM HDAC1 (His/FLAG; 50051; BPS Biosciences Inc), 2.5 nM Tb-Anti-6×His IgG, 2 h incubation. (b) Dose-response curves for HDAC inhibitor test set as measured in TR-FRET assay with recombinant HDAC1. Conditions: 5 nM HDAC1 (His/FLAG; 50051; BPS Biosciences Inc), 2.5 nM Tb-Anti-6×His IgG, 20 nM SAHA-NCT or 70 nM M344-FITC, 3 h incubation. (c) HDAC activity dose-response curves for HDAC inhibitor test set, as well as fluorescent HDAC tracers (SAHA-NCT, M344-FITC) used in this study toward recombinant HDAC1. Conditions: 5 nM HDAC1 (His/FLAG; 50051; BPS Biosciences Inc), 18 AM MAZ1600 substrate (3×KM), 3 h incubation. See Table herein for measured IC₅₀ and determined K_{d} values. Data are represented as means±SD of three replicates (n=3).

FIG. 46 Profiling cellular response of HDAC inhibitors with 0.25 UM SAHA-NCT. Cellular dose-response curves for HDAC inhibitor test set as measured in TR-FRET assay with Cora-2-Halo labeled HEK293T cells expressing HDAC1-HaloTag, with 0.25 μM SAHA-NCT tracer present. Conditions: 25,000 cells/well (384-well plate; Corning 3574), 4 h incubation at 37° C. and 5% CO₂. See Table herein for measured EC₅₀ and apparent Kᵢ (K_{i,app}) values. Data are represented as means±SD of six replicates (n=6).

FIG. 47A schematically shows assay for proteins of interest with two or more binding sites.

FIG. 47B schematically shows assay for proteins of interest with multiprotein complex.

FIG. 48 schematically shows assay for membrane-bond proteins of interest.

FIG. 49 Quantification of BRD4 protein in MCF7-cell lysate: Condition 1) 1 nM Ab, 2 nM Tb-nano-secondary, 20 nM JQ1-FITC, 2) 0.5 nM Ab, 1 nM Tb-nano-secondary, 10 nM JQ1-FITC, 3) 0.25 nM Ab, 0.5 nM Tb-nano-secondary, 5 nM JQ1-FITC, 4) 0.125 nM Ab, 0.25 nM Tb-nano-secondary, 2.5 nM JQ1-FITC, 5) 10 nM JQ1 mix, 6) 5 nM JQ1 mix, 7) 2.5 nM JQ1 mix, 8) 1.25 nM JQ1 mix.

FIG. 50 Quantification of BRD4 protein in MCF7-cell lysate following dBET6 treatment: MCF7 cells were seeded at 600 k/well in 6-well plates and allowed to recover overnight. Cells treated either with DMSO (0.25%) or 250 nM dBET6 for 5 h. Cells were washed and lysed in 250 μL lysis buffer/well for 30 min at 4 C. Insoluble matter was removed by centrifugation. Cleared lysate was added (30 μL) to 384-well plate and then add 7×detection mix 0.5 nM rabbit anti-BRD4, 1 nM rabbit-nano secondary-Tb, 10 nM JQ1-FITC

FIG. 51 Cell-membrane targeted TR-FRET: a) HEK293T cell were incubated in the presence and absence of DiI (3 μM) with a Tb-labeled anti CD44 antibody (10 nM), or with a Tb-labeled anti-GST antibody (10 nM) in the presence of DiI (background control). B) structures of MCP415 and NCP189 Tb-complexes, c) MCF7 cells were incubated with 1 μM NCP415 or NCP189 in the presence of DiI.

FIG. 52 HDAC isoform and complex specific assays for lysate and cell-based applications. The HDAC isoform (A) of interest or a member protein (B) of a specific HDAC complex is expressed as Halo Tag fusion protein for labeling with an Halo Tag-ligand functionalized CoraFluor or alternative an CoraFluor-labeled specific antibody is used to install the TR-FRET donor. A fluorophore tagged HDAC inhibitor will be used as TR-FRET acceptor. This approach will then be used in a ligand displacement assay for small molecule inhibitor profiling. The tag-free approach was be validated using the pairwise combination of CoraFluor- and acceptor-functionalized HDAC inhibitors.

### DETAILED DESCRIPTION

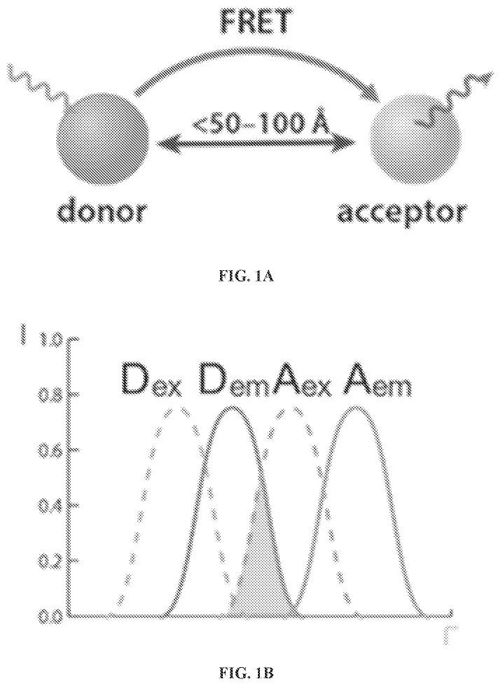

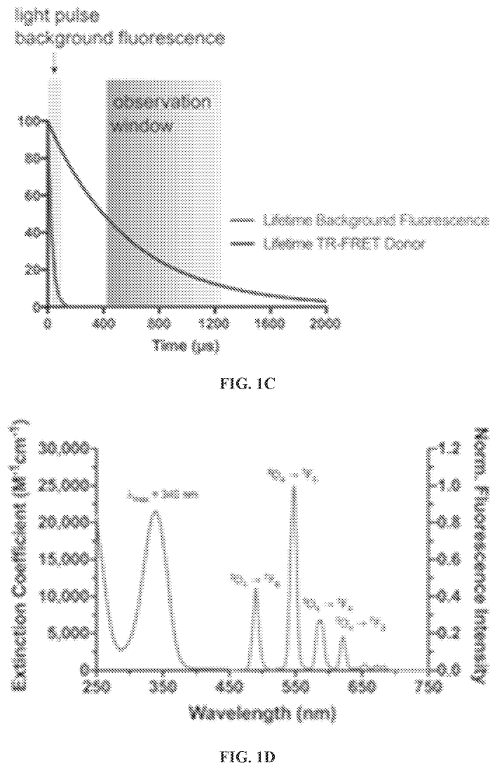

All assay platforms that are discussed in this invention disclosure combine time-resolved (TR) fluorescence measurements with Förster resonance energy transfer (FRET, FIG. 1). TR-FRET-based assays stand out with superior sensitivity, unparalleled flexibility, and assay robustness. In TR-FRET assays, the signal is generated by energy transfer from a donor with a long luminescence lifetime to an acceptor fluorophore when in close proximity to each other (within 5-10 nm, approximately the size of a nucleosome). The time gated measurement allows for the virtual elimination of non-specific background signals originating from scattered excitation light and autofluorescence of screening compounds, buffer reagents and assay plates, while the FRET component limits the readout to acceptor molecules that are in immediate proximity of the donor. This approach therefore enables the quantitative measurement of the interaction of biomolecules and/or small molecule ligands with superior sensitivity.

TR-FRET assays are target agnostic and many TR-FRET based assays have been published and/or are commercially available. Most TR-FRET assay platforms are ligand displacement assays that measure the disruption of a TR-FRET pair and not the enzymatic turnover of a substrate (e.g. fluorogenic or luminescent) that generates a specific signal. This strategy allows for a real-time readout and is beneficial for POI that lack enzymatic activity or have inherently low turnover rates.

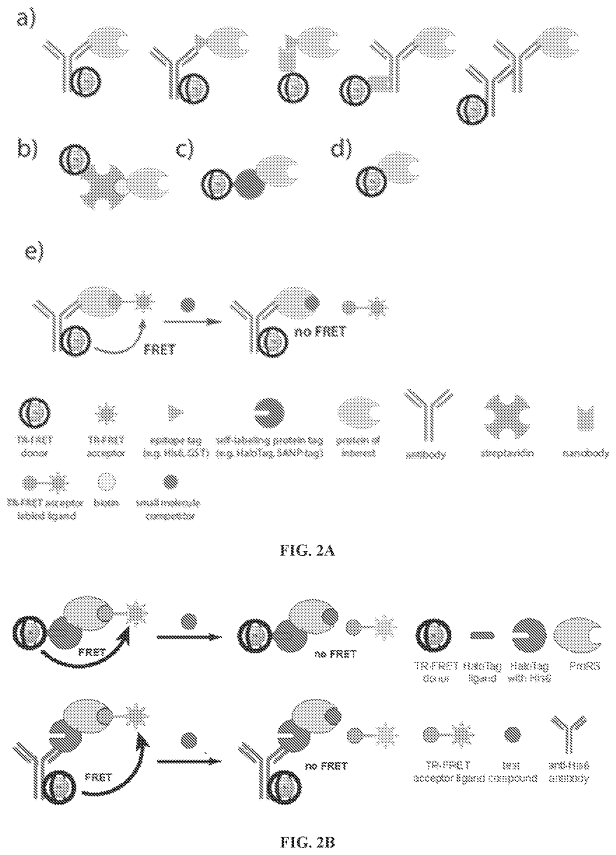

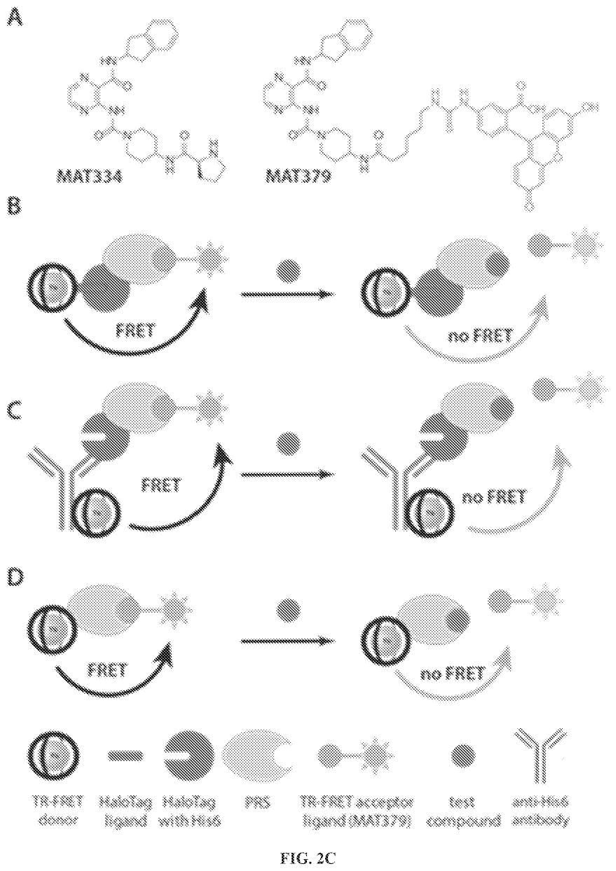

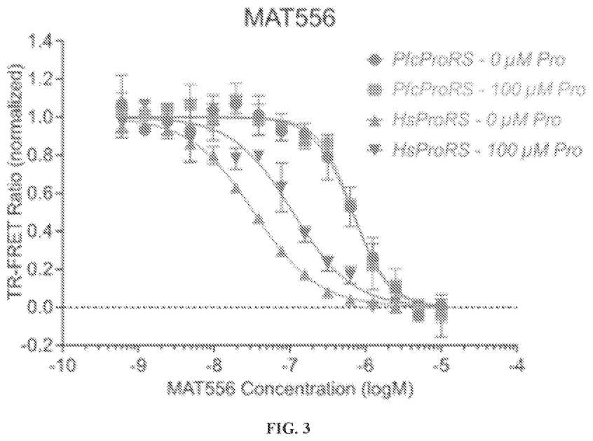

TR-FRET assays generally follow the same canonical scheme (FIG. 2A). The POI is tagged with a TR-FRET donor (generally a luminescent terbium or europium complex) using a) a donor-modified specific antibody or nanobody, or combination thereof, b) donor-modified streptavidin if the POI is biotinylated, c) functionalized via a self-labeling protein tag such as HaloTag, SNAP-tag or CLIP-tag, d) or directly covalently labeled by reaction of lysine and cysteine side chains with active ester (e.g. NHS-esters, isothiocyanates) and maleimide-functionalized donors, respectively. If the assay is designed to study protein-small molecule interaction, a small molecule ligand with sufficiently high affinity labeled with a suitable fluorophore to function as FRET acceptor is used as a tracer. The tracer is then incubated at fixed concentration with the donor-tagged POI in the presence of varying concentrations of test compound. Measurement of the dose-dependent change of the TR-FRET signal enables determination of the binding affinity of the test compounds. While the positions of acceptor and donor label can be switched, the high costs and limited availability of conjugatable TR-FRET donors are generally prohibitive for small molecule labeling. If the assay is designed to measure a protein-protein interaction, both binding partners are orthogonally tagged with a TR-FRET donor and acceptor, respectively. In some cases, fluorescent proteins can be used as acceptors instead of small molecule fluorophores. To determine specific posttranslational modifications or the abundance of a POI, orthogonally labeled complementary antibody pairs are used.

### Exemplary Assay Platforms

In some embodiments, the present disclosure provides assays for studying interactions between a modulator compound of a protein of interest and the protein of interest itself. For example, the assay can be used to determine binding and other characteristics between the compound and the protein, or to determine concentration of the protein in a sample (e.g., aqueous buffer, live cells, or cell lysate). In another example, the assay can be used to identify novel modulators of the protein, for example, using a ligand displacement strategy. In some embodiments, the cell is implicated in the pathologies of a disease or conditions (e.g., any of the diseases described herein). For example, the cell is a cancer cell or a brain cell affected by a neurodegenerative condition.

Accordingly, in some embodiments, the present disclosure provides a method of identifying a compound that modulates a protein of interest, the method comprising:
(i) providing a sample comprising a protein of interest attached to a FRET donor moiety and a ligand attached to a FRET acceptor moiety, said ligand capable of binding to the protein of interest;
(ii) exciting the sample with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique;
(iii) contacting the sample with a test compound;
(iv) after (iii), detecting a light of a wavelength emitted by the FRET acceptor moiety by a fluorescence imaging technique; and
(v) determining whether the intensity of fluorescence detected from the FRET acceptor moiety in step (iv) is decreased compared to the intensity of fluorescence detected from the FRET acceptor moiety in step (ii),
wherein said decrease in fluorescence intensity in an indication that the test compound is the modulator the protein of interest.

In some embodiments, the method is a high-throughput screening method. The method can be carried out using suitable screening robots handling multiwall assay plates. The plates can be made of glass or plastic or any other suitable material that allows for FRET applications (such as a material that allows the excitation light to reach the FRET donor and allows the fluorescence from the FRET acceptor to reach the measuring device). The plate may contain 96, 192, 384, 1536, 3456, or 6144 wells as appropriate. A skilled chemist or an engineer would be able to select and implement appropriate HTS equipment. The sample provided in step (i) may be placed, for example, in one of the wells of the multiwall plate.

Step (i) can be carried out, for example, by obtaining a protein of interest attached to FRET donor (as discussed below) and admixing it with the ligand attached to FRET acceptor moiety (obtained as discussed below). The two components may be allowed to equilibrate for a period of time sufficient for the ligand to bind to the protein of interest. For example, the sample can be equilibrated for about 10 min, about 30 min, about 1 hour, about 2 hours, or about 3 hours. The sample may also contain a carrier liquid, such as water or a buffer solution to facilitate the binding. A concentration of the protein of interest in the sample may range from about 1 μM to about 1 μM, or from about 1 μM to about 1 nM. In some embodiments, the ligand attached to FRET acceptor is any one of the tracer compounds of Formulae (I)-(VII), or a pharmaceutically acceptable salt thereof. Concentration of the tracer in the sample may also range from about 1 μM to about 1 μM, or from about 1 μM to about 1 nM.

In some embodiments, in step (ii), said detecting of fluorescence may be carried out using fluorescent microscopy, fluorescent imaging probe, or fluorescent spectroscopy. In one example, both the excitation of the donor and detection (and measurement) of fluorescence of the acceptor can be performed using a single piece of equipment. Excitation can be carried out using a UV lamp or a laser. For detecting fluorescence, either photomultiplier (PMT) or charge-couple device (CCD) can be used to detect and quantify emitted photons. Also, total internal reflection fluorescence microscopy, light sheet fluorescence microscopy, or fluorescence-lifetime imaging microscopy can be used. The amount of time between exciting the FRET donor and reading fluorescence output from FRET acceptor can be from about 1 sec to 10 min, from about 5 sec and about 5 min, from about 10 sec to about 2 min, or from about 30 sec to about 1 min. In some embodiments, the wavelength of the light capable of being absorbed by the FRET donor moiety is from about 300 nm to about 400 nm. In some embodiments, the wavelength emitted by the FRET acceptor moiety is from about 450 nm to about 600 nm. A skilled analytical chemist would be able to tune the equipment as necessary depending to the particular excitation and fluorescent characteristics of the FRET pair used in the method.

Step (iii) can be carried out by adding a solution of a test compound or compounds to the sample provided in step (i). For example, a solution of the test compound of suitable concentration (from about 1 μM to about I μM) in an aqueous solvent or an organic solvent such as DMSO can be pipetted to the sample manually or robotically.

In some embodiments, the compound is an inhibitor of a protein of interest (substrate-competitive orthosteric inhibitor, substrate-noncompetitive orthosteric inhibitor, or allosteric inhibitor). In some embodiments, the test compound is an antagonist or a partial antagonist of the function of the protein. In some embodiments, the compound is an activator of a protein of interest. In some embodiments, the test compound is an agonist or a partial agonist of the protein of interest.

In some embodiments, affinity of the ligand attached to the FRET acceptor moiety to the protein of interest is less than affinity of the test compound to the protein of interest. For example, affinity of the ligand may be from about 100 to about 200 nM, while affinity of the test compound may be from about 10 nM to about 100 nM. In another example, affinity of the test compound is about 2×, about 4×, about 10×, about 20×, about 50×, about 100×, or about 200×greater compared to affinity of the ligand.

Step (iv) may be carried out in a manner similar to step (ii) above, by detecting and, if necessary, quantifying the fluorescence signal using a microscopy or spectroscopy device and associated software. The following step (v) of comparing the fluorescence intensity of step (iv) and the fluorescence intensity of step (ii) can be carried out using any suitable device or a piece of software. Without being bound by any particular theory, it is believed that the decrease in the intensity of fluorescence signal in step (iv) compared to step (ii) indicates that the test compound has bond to the protein of interest and thereby displaced the fluorescent tracer, which is in turn indicative of the fact that the test compound is a modulator of the protein of interest. In some embodiments, the test compound has the same mode of action as the tracer (e.g., the test compound and the tracer are both inhibitors of the protein of interest). In other embodiments, the test compound and the tracer have different mode of action (e.g., the test compound is a substrate-competitive inhibitor and the tracer is allosteric inhibitor). In some embodiments, the fluorescence intensity in step (iv) is about 2×, about 4×, about 5×, about 10×, about 20×, about 50×, or about 100×less than in step (ii).

### FRET Acceptor Moieties

In some embodiments, the fluorescent FRET acceptor moiety is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red. The wavelength of emitted light (e.g., maximum of emittance) for each of these FRET acceptor moities is well-known in the literature. Depending on the acceptor chosen, the chemist can adjust the selection of FRET donor and the various parameters of the step (ii) process, e.g., to ensure that the energy can be efficiently transferred from the donor moiety or the acceptor moiety.

In some embodiments, the FRET acceptor moiety has formula:

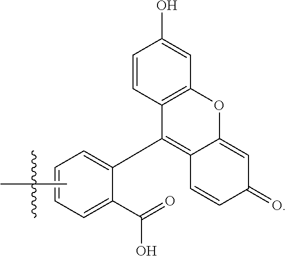

### FRET Donor Moieties

In some embodiments, the FRET donor moiety comprises a complex of a lanthanide metal with a moiety of formula (i):

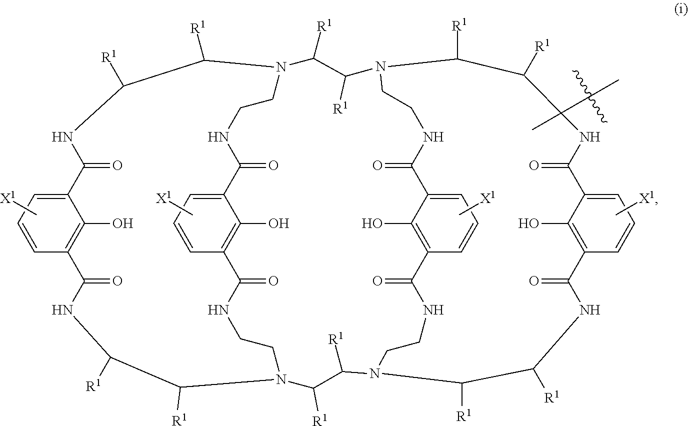

wherein:
each X¹ is independently selected from halo, NO₂, CN, N₃, C₁₋₆ alkyl, C₁₋₆ alkoxy, C₂₋₆ alkenyl, C₂₋₆ alkynyl, C₆₋₁₀ aryl, and 5-14 membered heteroaryl, wherein said C₁₋₆ alkyl, C₂₋₆ alkenyl, C₂₋₆ alkynyl, C₆₋₁₀ aryl, and 5-14 membered heteroaryl are each optionally substituted with 1, 2, or 3 substituents independently selected from halo, OH, SH, NH₂, C₁₋₃ alkylamino, di (C₁₋₃ alkyl) amino, NO₂, CN, C (O)OH, C₁₋₃ alkoxy, C₁₋₃ haloalkoxy, and N₃; and
each R¹ is independently selected from H, C₁₋₆ alkyl, C₂₋₆ alkenyl, and C₂₋₆ alkynyl, each of which is optionally substituted with 1, 2, or 3 substituents independently selected from halo, OH, SH, NH₂, C₁₋₃ alkylamino, di (C₁₋₃ alkyl) amino, NO₂, CN, C (O)OH, Cis alkoxy, C₁₋₃ haloalkoxy, and N₃.

In some embodiments, [symbol] indicates a point of attachment of the donor moiety to either the protein of interest (e.g., to one of the side chains of an amino acid within the protein of interest), the antibody or nanobody, or the linker connecting the donor with protein, the antibody or nanobody, or a tag ligand, such as a halotag ligand. [symbol] also may indicate a point of attachment of the donor to streptavidin.

In some embodiments, each X¹ is independently a H or a halo.

In some embodiments, each X¹ is H.

In some embodiments, each X¹ is C₁ or Br.

In some embodiments, one of RI is selected from C₁₋₆ alkyl, C₂₋₆ alkenyl, and C₂₋₆ alkynyl, each of which is optionally substituted with 1, 2, or 3 substituents independently selected from halo, OH, SH, NH₂, C₁₋₃ alkylamino, di (C₁₋₃ alkyl) amino, NO₂, CN, C (O)OH, C₁₃ alkoxy, C₁₃ haloalkoxy, and N₃; and the remaining R¹ groups are all H.

In some embodiments, each R¹ is H.

In some embodiments, the moiety of formula (i) has formula:

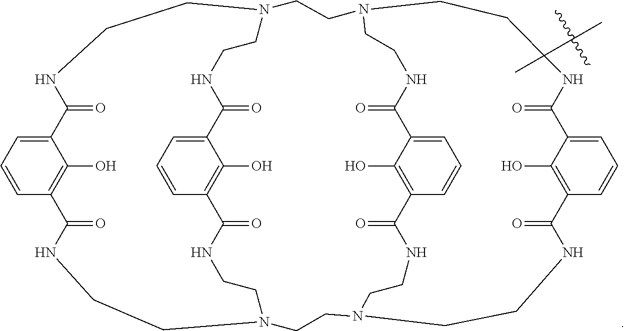 [SMILES: *C1(C)CCN2CCNC(=O)c3cccc(c3O)C(=O)NCCN(CCNC(=O)c3cccc(c3O)C(=O)N1)CCN1CCNC(=O)c3cccc(c3O)C(=O)NCCN(CCNC(=O)c3cccc(c3O)C(=O)NCC1)CC2]

In some embodiments, the moiety of formula (i) has formula:

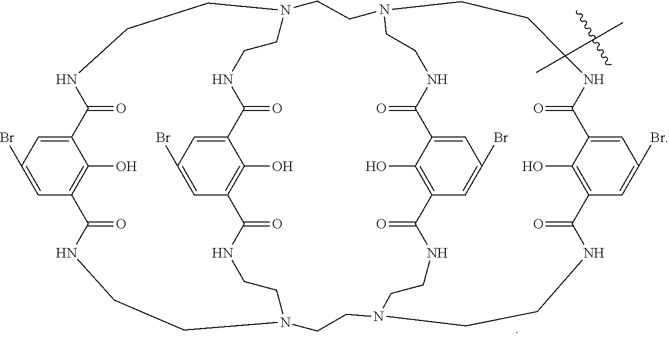 [SMILES: *C1(C)CCN2CCNC(=O)c3cc(Br)cc(c3O)C(=O)NCCN(CCNC(=O)c3cc(Br)cc(c3O)C(=O)N1)CCN1CCNC(=O)c3cc(Br)cc(c3O)C(=O)NCCN(CCNC(=O)c3cc(Br)cc(c3O)C(=O)NCC1)CC2]

In some embodiments, the moiety of formula (i) has formula:

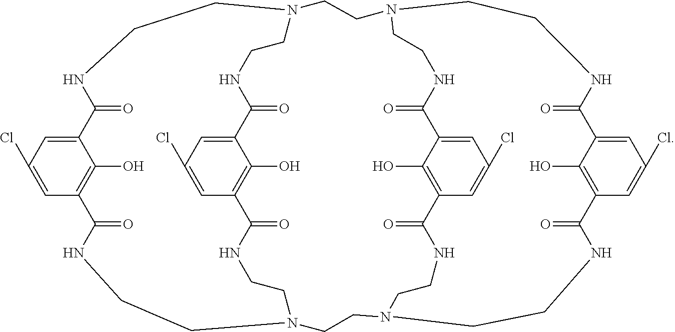 [SMILES: O=C1NCCN2CCNC(=O)c3cc(Cl)cc(c3O)C(=O)NCCN(CCNC(=O)c3cc(Cl)cc1c3O)CCN1CCNC(=O)c3cc(Cl)cc(c3O)C(=O)NCCN(CCNC(=O)c3cc(Cl)cc(c3O)C(=O)NCC1)CC2]

In some embodiments, the lanthanide metal is selected from Tb (terbium), Eu (europium), Sm (samarium), and Dy (dysprosium). In some embodiments, the lanthanide metal is Tb³⁺.

In some embodiments, the protein of interest is selected from an enzyme, a cell-surface receptor, nuclear hormone receptor, a transporter, a G-protein coupled receptor, a CD marker, a voltage-gated ion channel, a nuclear factor, a nuclear receptor, a protein-protein or protein-peptide interaction domain, scaffolding protein, structural protein, transcription factor, chaperone, and assembly/disassembly factor.

In some embodiments, the enzyme is selected from kinases, proteases, deacetylases, ATPases, GTPases, phosphatases, peptidases, synthetases, phosphorilases, and nucleosidases.

In some embodiments, the protein of interest is selected from KEAP1 protein, bromodomain protein, and an aminoacyl tRNA synthetase. In some embodiments, the protein of interest is selected from KEAP1 protein, bromodomain protein, an aminoacyl tRNA synthetase, and a histone deacetylase (e.g., HDAC1, 2, 3, 4, 5, 6, 7, 8, 9, or 10).

Suitable examples of attaching FRET donor moiety are schematically shown in FIGS. 2A, 2B, and 2C.

In some embodiments, the method includes making the protein of interest attached to a FRET donor moiety in the sample by contacting the protein of interest comprising a halotag with a FRET donor moiety comprising a halotag ligand. For example, the protein of interest can be expressed as a fusion protein with halotag optionally with His6).

In some embodiments, the method includes making the protein of interest attached to a FRET donor moiety by contacting the protein of interest comprising an epitope tag with an antibody or nanobody to the epitope tag, the antibody or nanobody being attached to the FRET donor moiety (directly or through a linker).

In some embodiments, the method includes making the protein of interest attached to a FRET donor moiety by contacting the protein of interest with an antibody or nanobody to the protein of interest, the antibody or nanobody being attached to the FRET donor moiety.

In some embodiments, the method includes making the protein of interest attached to a FRET donor moiety by contacting the protein of interest with a first antibody or nanobody to the protein of interest to obtain the protein-antibody conjugate, followed by contacting the conjugate with a second antibody or nanobody to the first antibody or nanobody, the second antibody or nanobody being attached to the FRET donor moiety (directly or through a linker).

In some embodiments, the method includes making the protein of interest attached to a FRET donor moiety by contacting the protein of interest comprising a biotin moiety with a streptavidin protein attached to the FRET donor moiety.

In some embodiments, the method includes making the protein of interest attached to a FRET donor moiety by contacting the protein of interest with a FRET donor moiety comprising an activated ester. In some embodiments, the activated ester is selected from N-hydroxysuccinimide, sulfo-N-hydroxysuccinimide, tetrafluorophenoxy, pentafluorophenoxy, and p-nitophenoxy.

In some embodiments, the method includes making the ligand attached to the FRET acceptor moiety by coupling the ligand with the FRET acceptor moiety using a linker moiety.

In some embodiments, the protein of interest is an aminoacyl tRNA synthetase and the ligand attached to the FRET acceptor moiety is a tracer compound of Formula (I) described herein, or a pharmaceutically acceptable salt thereof, wherein the amino acid in the compound of Formula (I) corresponds to the amino acid attached to the RNA by the aminoacyl tRNA synthetase enzyme.

Amino acyl sulfamoyl adenosine such as prolyl-sulfamoyl adenosine (ProSA), have been shown to be high affinity ligands for their respective aaRS isoforms. aaSA closely mimic amino acyl AMP (e.g. prolyl-AMP), the activated amino acid intermediate that is formed in the first catalytic step by reaction of the cognate amino acid and ATP. aaSA analogs are hydrolytically stable and therefore can be employed as tool compounds to selectively inhibit aaRS activity. Corresponding nitrogen-analogs amino acyl sulfamoyl amino adenosine (aaSNA) offer the possibility for linker attachment sufficiently close to the narrow aperture that is otherwise occupied by the triphosphate of ATP.

In some embodiments, the protein of interest is a prolyl tRNA synthetase and the ligand attached to the FRET acceptor moiety is a tracer compound of Formulae (II) or (III), or a pharmaceutically acceptable salt thereof.

In some embodiments, the protein of interest is a phenylalanyl tRNA synthetase and the ligand attached to the FRET acceptor moiety is a tracer compound of Formula (IV), or a pharmaceutically acceptable salt thereof.

In some embodiments, the protein of interest is a isoleucyl tRNA synthetase and the ligand attached to the FRET acceptor moiety is a tracer compound of Formula (VI), or a pharmaceutically acceptable salt thereof.

In some embodiments, the protein of interest is a glutamyl-tRNA synthetase and the ligand attached to the FRET acceptor moiety is a tracer compound of Formula (VII), or a pharmaceutically acceptable salt thereof.

In some embodiments, the sample comprises live cells (e.g., human cells, bacterial cells, or parasite cells, as may be appropriate).

In some embodiments, the sample comprises a cell lysate. The sample may contain surfactants used to lyse cells, or the sample may be surfactant-free.

In some embodiments, the sample comprises an aqueous solution. In some embodiments, the aqueous solution is a buffer solution (having pH from about 5 to about 8).

### Evaluating and Studying Ligands

In some embodiments, the present disclosure provides a method of evaluating an interaction between a protein of interest and a modulator of the protein of interest, the method comprising:
(i) providing a sample comprising the protein of interest attached to a FRET donor moiety and the modulator attached to a FRET acceptor moiety;
(ii) exciting the sample with a light of a wavelength capable of being absorbed by the FRET donor moiety, followed by waiting an amount of time sufficient for energy transfer from the FRET donor moiety to the FRET acceptor moiety; and
(iii) detecting a light of a wavelength emitted by the FRET acceptor moiety by a fluorescence imaging technique;
wherein the fluorescence intensity detected from the FRET acceptor moiety is indicative of quality and/or quantity of the interaction between the modulator and the protein of interest.

In some embodiments, the steps (i)-(iii) are carried out as described for the screening method above. The embodiments of the protein, FRET donors, FRET acceptors, and equipment are also as in the screening method.

In some embodiments, the method includes determining a thermodynamic binding constant between the modulator and the protein of interest.

In some embodiments, the method includes determining a kinetic binding constant between the modulator and the protein of interest.

In some embodiments, the method includes determining the mode of binding of the modulator to the protein of interest.

In some embodiments, the method includes determining whether the modulator is a substrate-competitive orthosteric inhibitor, substrate-noncompetitive orthosteric inhibitor, or allosteric inhibitor.

In some embodiments, the method includes determining whether the modulator is an activator of the protein of interest.

In some embodiments, the method includes determining binding affinity between the modulator and the protein of interest.

In some embodiments, the method includes making the modulator attached to a FRET acceptor moiety by coupling the modulator to the FRET acceptor moiety through a linker.

### Quantification Methods

In some embodiments, the present disclosure provides a method of determining an amount of a protein of interest in a sample, the method comprising:
(i) providing the sample comprising the protein of interest attached to a FRET donor moiety and the modulator attached to a FRET acceptor moiety;
(ii) exciting the sample with a light of a wavelength capable of being absorbed by the FRET donor moiety, followed by waiting an amount of time sufficient for energy transfer from the FRET donor moiety to the FRET acceptor moiety; and
(iii) detecting a light of a wavelength emitted by the FRET acceptor moiety by a fluorescence imaging technique;
wherein the fluorescence intensity detected from the FRET acceptor moiety is correlated with the amount of the protein of interest in the sample.

In some embodiments, the steps (i)-(iii) are carried out as described for the screening method above. The embodiments of the protein, FRET donors, FRET acceptors, and equipment are also as in the screening method.

In some embodiments, the method comprises quantifying abundance of a post-translational modification of the protein of interest.

In some embodiments, the method comprises determining intracellular concentration of the protein of interest.

In some embodiments, the method includes obtaining a sample comprising the protein of interest from a subject for diagnosing a disease or condition, wherein the amount of the protein of interest in the sample is indicative of the disease or condition.

In some embodiments, the method includes obtaining a sample comprising the protein of interest from a subject for monitoring treatment a disease or condition, wherein the amount of the protein of interest in the sample is indicative of efficacy of treatment of the disease or condition.

In some embodiments, the sample comprises live cells.

In some embodiments, the sample comprises cell lysate.

In some embodiments, the protein of interest in implicated in the disease or condition. Suitable examples of such proteins include proteins implicated in the pathology of cancer. Suitable example of such proteins include kinases (cytosolic and receptor), transcription factors, epigenetic writers (e.g., methyltransferases, acetyltransferases.) epigenetic readers, and epigenetic erasers (e.g., demethylases, deacetylases). Examples of methyltransferases include those described in Nature Structural & Molecular Biology volume 26, pages 880-889 (2019), which is incorporated herein by reference in its entirety. Examples of histone demethylases include those described in Nature Reviews Molecular Cell Biology volume 13, pages 297-311 (2012), which is incorporated herein by reference in its entirety. More specifically, suitable examples of such proteins include hormone receptor, androgen receptor (AR), estrogen receptor (ER), estrogen-related receptor alpha (ERRα), KRAS, BRD4 (bromodomain and extraterminal (BET) domain epigenetic reader protein BRD4), BRD2, BRD3, anaplastic lymphoma kinase (ALK), BCL2, BCL6, BCR-ABL, BRD9, BRD7, BTK, CDK4/6, cyclin-dependent kinase 8 (CDK8), cyclin-dependent kinase 9 (CDK9), casein kinase 2 (CK2), c-Met, dihydroorotate dehydrogenase (DHODH), epidermal growth factor receptor (EGFR), human epidermal growth factor receptor 2 (HER2), eukaryotic translation initiation factor 4E (CIF4E), ERK1, ERK2, focal adhesion kinase (FAK), FMS-like tyrosine kinase 3 (FLT3), myeloid cell leukemia 1 (MCL1), murine double minute 2 (MDM2), poly (ADP-ribose) polymerase (PARPs, such as PARPI), transforming acidic coiled-coil containing protein 3 (TACC3), pirin, phosphoinositide 3-kinases (P13Ks), polycomb repressive complex 2 (PRC2), serine-threonine kinase (RIPK2), rpn13, serum/glucocorticoid-inducible protein kinase (SGK), smad3, STAT protein (STAT1, STAT2, STAT3, STAT4, STATSA, STAT5B, or STAT6), TANK-binding kinase 1 (TBKI), TRIM24, the hepatitis C virus (HCV) NS3 protein, interleukin-1 receptor-associated kinase 4 (IRAK4), P300/CBP-associated factor (PCAF), cellular retinoic acid-binding protein (CRABP-I, -II), anaplastic lymphoma kinase (ALK), mitogen-activated protein kinase 14 (MAPK14, p38-α), mitogen-activated protein kinase 13 (MAPK 13, also known as stress-activated protein kinase 4 (SAPK4), or p38-δ), sirtuin, sirtuin2 (SIRT2), P300/CBP associating factor (PCAF), histone deacetylase (e.g., HDAC1, HDAC2, HDAC3, HDAC4, HDAC5, HDAC6,m HDAC7, HDAC8, HDAC9, HDAC10, or HDAC11), cytosolic aminoacyl tRNA synthetase, mitochondrial aminoacyl tRNA synthetase, PD-L1, CD47, cytokine (e.g., IL-2, IL-7, IL-12, IL-15, IL-10, IL-21, or INF-alfa), chemokine (e.g., CCL2, CCL3, or CCL5), and an immunosuppressive antigen (e.g., PD-1, CTLA-4, CD20, Lag-3 or Tim-3). In some embodiments, the protein is implicated in the pathology of a neurodegenerative disease or condition. Suitable examples of such proteins include alpha-synuclein, transthyretin, tau protein, and amyloid-β peptide.

In some embodiments, the assay platforms described herein can be used to identify a PROTAC compound, and/or to study intractions of the compounds with the protein of interest, including quantitatively and qualitavely. The assays can be used to identify the ligase recruiting ligand and/or to identify a protein targeting ligand.

In some embodiments, the protein of interest comprises 1, 2, 3, 4, or 5 binding sites (e.g., 2 or more binding sites). In some embodiments, the POI comprises 1 or 2 binding sites. In some embodiments, each of the two or more binding sites can bind the FRET donor moiety-containing reagent and the FRET acceptor moiety containing reagent. In some embodiments, the POI is a multi-protein complex. For example, the POI may comprises 2, 3, 4, 5, or 6 protein domains. In some embodiments, FRET donor moiety and FRET acceptor moiety are bound to different domains within the complex.

In some embodiments, the present disclosure includes a method of identifying a PROTAC compound, the method comprising:
(i) providing a sample comprising a protein of interest attached to a FRET donor moiety and a modulator attached to a FRET acceptor moiety, said ligand capable of binding to the protein of interest;
(ii) exciting the sample with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique;
(iii) contacting the sample with a test compound (e.g., test PTOTAC compound);
(iv) after (iii), exciting the sample with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique;
(v) determining whether the intensity of fluorescence detected from the FRET acceptor moiety in step (iv) is decreased compared to the intensity of fluorescence detected from the FRET acceptor moiety in step (ii),
wherein said decrease in fluorescence intensity in an indication that the test compound is the PROTAC compound capable of degrading the protein of interest (e.g., using the proteasome machinery of the cell).

In some embodiments, the protein of interest is targeted by PROTAC compound for degradation. The modulators, tracers, FRET donors and acceptors, as well as the methods of carrying out steps (i)-(v) are as described herein.

The present disclosure also provides assay platforms for identifying modulators of transmembrane proteins of interest, as well as methods of studying transmembrane proteins of interests quantitatively and qualitatively.

In some embodiments, the present disclosure includes a method of identifying a compound that modulates a transmembrane protein of interest, the method comprising:
(i) providing a cell comprising a transmembrane protein of interest attached to a FRET donor moiety;
(ii) contacting the cell with a fluorophore capable of intercalating in a lipid bilayer of the membrane of the cell, the fluorophore capable of being a FRET acceptor moiety;
(ii) exciting a sample comprising the cell with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique;
(iii) contacting the sample with a test compound;
(iv) after (iii), exciting the sample with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique;
(v) determining whether the intensity of fluorescence detected from the FRET acceptor moiety in step (iv) is decreased compared to the intensity of fluorescence detected from the FRET acceptor moiety in step (ii),
wherein said decrease in fluorescence intensity in an indication that the test compound is the modulator the protein of interest.

In some embodiments, wherein affinity of the test compound to the protein of interest is greater than affibity of the FRET donor moiety to the protein of interest. The method can be carried out in a HTS manner as described herein. The method also can be used to monitor treatment of a disease. In this example, a sample containing a cell can be taken from a patient (e.g., blood, hair, tissue sample, biopsy, salive, urine, feces), and the test compounds is a drug the treatment with which is being monitored. The method can also be carried out in vivo, when the drug and the FRET donor, and the fluorophore acceptor are administered to the patient.

### Tracer Compounds

In some embodiments, the present disclosure provides a compound of Formula (I):

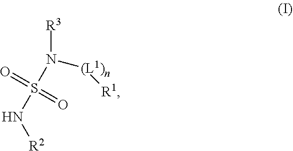

or a pharmaceutically acceptable salt thereof, wherein:
R³ is an ATP-binding moiety;
R² is an amino acid;
each L¹ is independently selected from O, S, S(═O)₂, NH, C═O, C═S, and C₁₋₆ alkylene;
n is an integer from 1 to 12; and
R¹ is a fluorophore.

In some embodiments, the ATP-binding moiety is selected from any one of the following moieties:

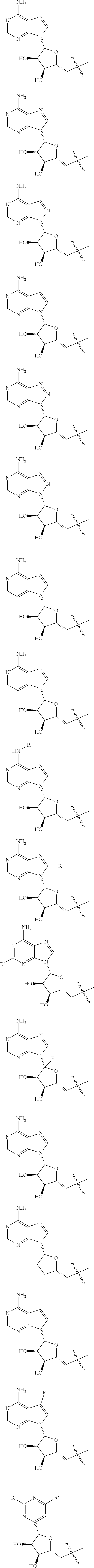

wherein R and R′ are independently selected from H, C₁₋₃ alkyl, and C₁₋₃ haloalkyl.

In some embodiments, the present disclosure provides a compound of Formula (I):

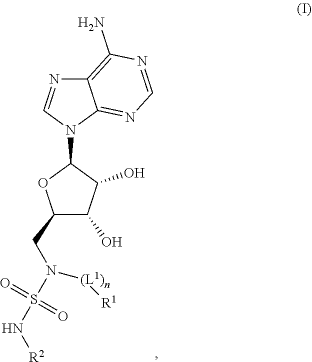

or a pharmaceutically acceptable salt thereof, wherein:
R² is an amino acid;
each L¹ is independently selected from O, S, S(═O)₂, NH, C═O, C═S, and C₁₋₆ alkylene;
n is an integer from 1 to 12; and
R¹ is a fluorophore.

In some embodiments, in the amino acid is selected from alanine, arginine, asparagine, aspartic acid, cysteine, glutamine, glutamic acid, glycine, histidine, isoleucine, leucine, isoleucine, lysine, methionine, phenylalanine, proline, serine, threonine, tryptophan, tyrosine, and valine.

In some embodiments, the amino acid is proline.

In some embodiments, the amino acid is phenylalanine.

In some embodiments, the amino acid is isoleucine.

In some embodiments, the amino acid is glutamic acid.

In some embodiments, each L¹ is independently selected from O, NH, C═O, C═S, and C₁₋₆ alkylene.

In some embodiments, the moiety (L¹)ₙ comprises (C═O)O.

In some embodiments, the moiety (L¹)ₙ comprises NH(C═O)O.

In some embodiments, the moiety (L¹)ₙ comprises (C═O)—C₁₋₆ alkylene.

In some embodiments, the moiety (L¹)ₙ comprises NH(C═S)NH.

In some embodiments, the moiety (L¹)ₙ comprises NH(C═O).

In some embodiments, the moiety (L¹)ₙ comprises NH(C═O) NH.

In some embodiments, the moiety (L¹)ₙ comprises OCH₂CH₂O.

In some embodiments, the moiety (L¹)ₙ comprises OCH₂CH₂NH.

In some embodiments, the compound has formula:

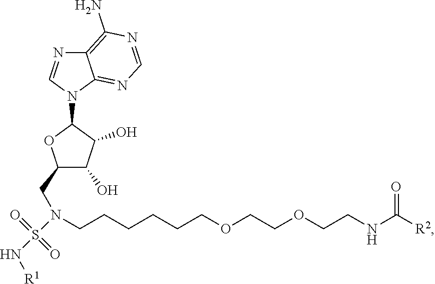

or a pharmaceutically acceptable salt thereof.

In some embodiments, R¹ is a FRET acceptor fluorophore. Suitable examples of fluorophores include any fluorescent chemical compounds that can re-emit light upon excitation. For example, the fluorophores in the compounds within the present claims are FRET acceptors. Without being bound by any particular theory, it is believed that the fluorophores may be excited by energy emitted through space by a FRET donor, and then emit light upon that excitation. In one example, the fluorophores can by excited by a light of a wavelength form about 300 nm to about 800 nm, and then emit light of a wavelength from about 350 nm to about 770 nm (e.g., violet, blue, cyan, green, yellow, orange or red light), which can be detected by fluorescent imaging devices, including the ability to measure the intensity of the fluorescence. Suitable examples of fluorophores include fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red. Absorbance and emission wavelengths of these fluorophores are well known in the art. Accordingly, in some embodiments, R¹ is selected from any of the aforementioned fluorophores, or a pharmaceutically acceptable salt thereof.

In some embodiments, the compound has formula:

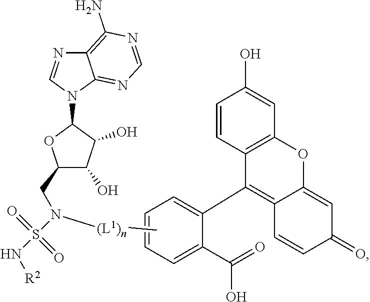

or a pharmaceutically acceptable salt thereof.

In some embodiments, the compound is selected from any one of the following compounds:

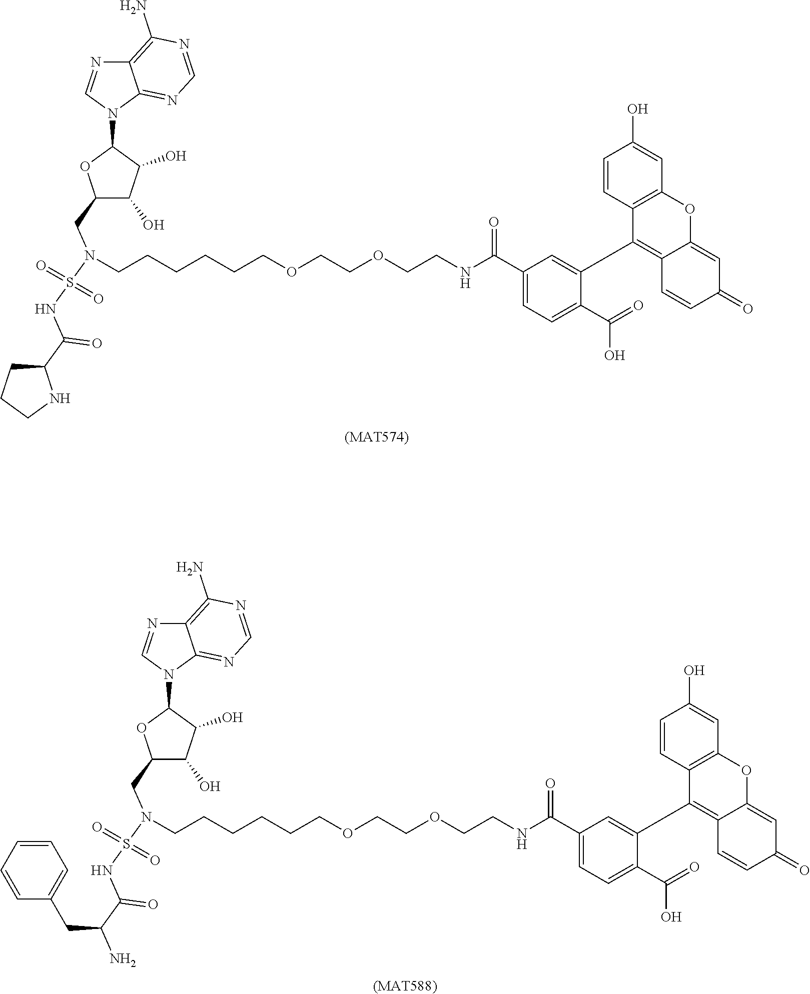 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H]2CCCN2)[C@@H](O)[C@H]1O ; "(MAT574)" ; Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H](N)Cc2ccccc2)[C@@H](O)[C@H]1O ; "(MAT588)"]

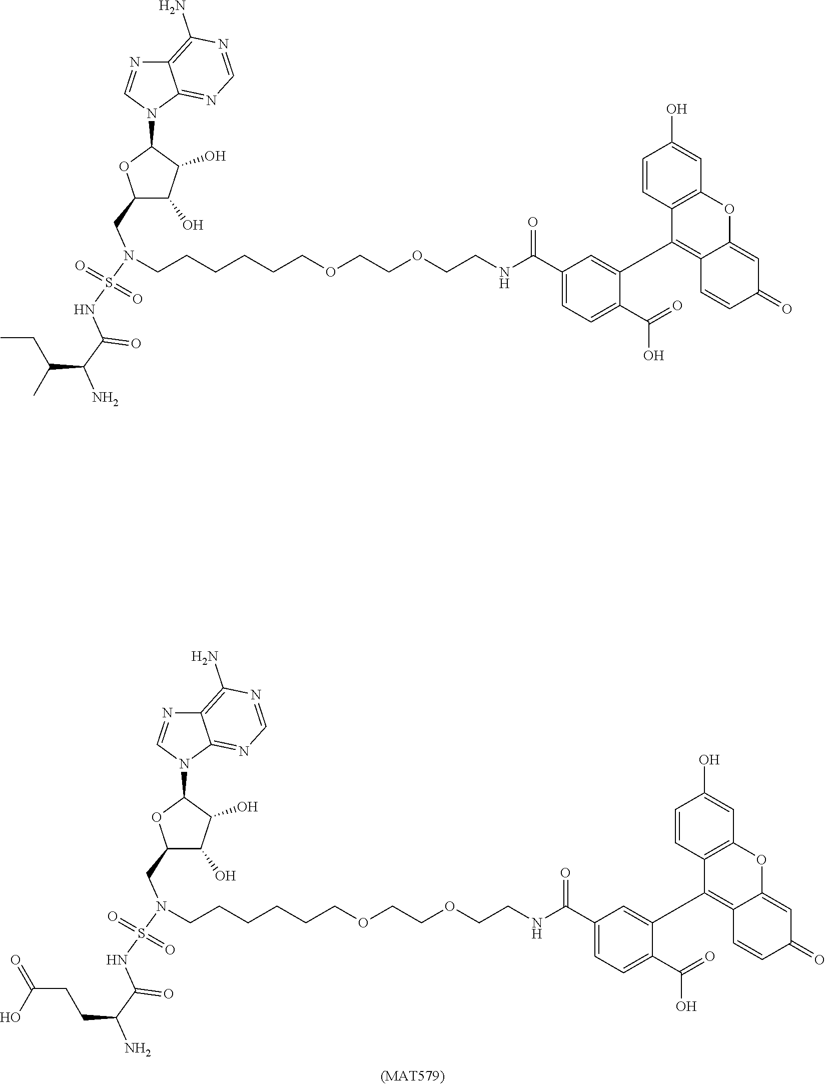 [SMILES: CCC(C)[C@H](N)C(=O)NS(=O)(=O)N(CCCCCCOCCOCCNC(=O)c1ccc(C(=O)O)c(-c2c3ccc(=O)cc-3oc3cc(O)ccc23)c1)C[C@H]1O[C@@H](n2cnc3c(N)ncnc32)[C@H](O)[C@@H]1O ; Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H](N)CCC(=O)O)[C@@H](O)[C@H]1O ; "(MAT579)"]

or a pharmaceutically acceptable salt thereof.

In some embodiments, this disclosure provides a compound of Formula (II):

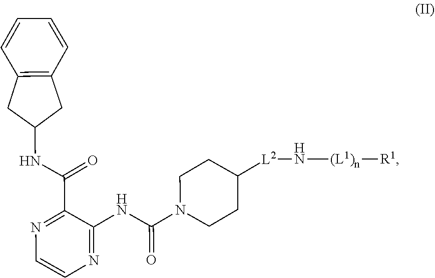

or a pharmaceutically acceptable salt thereof, wherein:
L² is C₁₋₃ alkylene; or L² is absent;
each L¹ is independently selected from O, S, S(═O)₂, NH, C═O, C═S, and C₁₋₆ alkylene;
n is an integer from 1 to 12; and
R¹ is a fluorophore.

In some embodiments, the compound has formula:

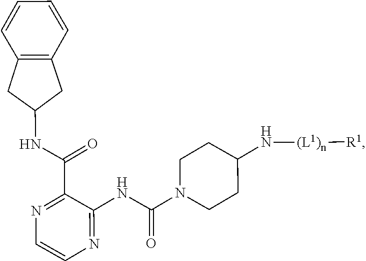

or a pharmaceutically acceptable salt thereof.

In some embodiments, the compound has formula:

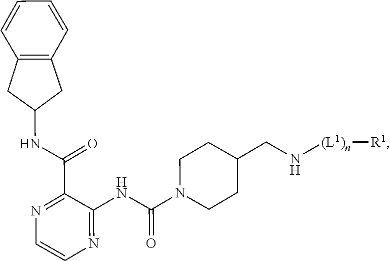

or a pharmaceutically acceptable salt thereof.

In some embodiments, each L¹ is independently selected from O, NH, C═O, C═S, and C₁₋₆ alkylene.

In some embodiments, the moiety (L¹)ₙ comprises (C═O)O.

In some embodiments, the moiety (L¹)ₙ comprises NH(C═O)O.

In some embodiments, the moiety (L¹)ₙ comprises (C═O)—C₁₋₆ alkylene.

In some embodiments, the moiety (L¹)ₙ comprises NH(C═S)NH.

In some embodiments, the moiety (L¹)ₙ comprises NH(C═O).

In some embodiments, the moiety (L¹)ₙ comprises NH(C═O) NH.

In some embodiments, the moiety (L¹)ₙ comprises OCH₂CH₂O.

In some embodiments, the moiety (L¹)ₙ comprises OCH₂CH₂NH.

In some embodiments, the moiety (L¹)ₙ comprises any one of the following fragments:

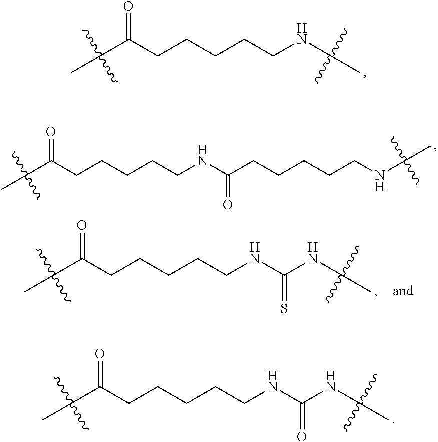 [SMILES: *NCCCCCC(*)=O ; *NCCCCCC(=O)NCCCCCC(*)=O ; *NC(=S)NCCCCCC(*)=O ; *NC(=O)NCCCCCC(*)=O]

In some embodiments, R¹ is a FRET acceptor fluorophore.

In some embodiments, R¹ is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.

In some embodiments, the compound has formula:

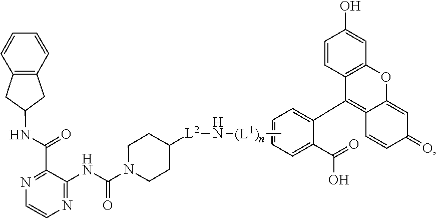

or a pharmaceutically acceptable salt thereof.

In some embodiments, the compound of Formula (II) is selected from any one of the following compounds:

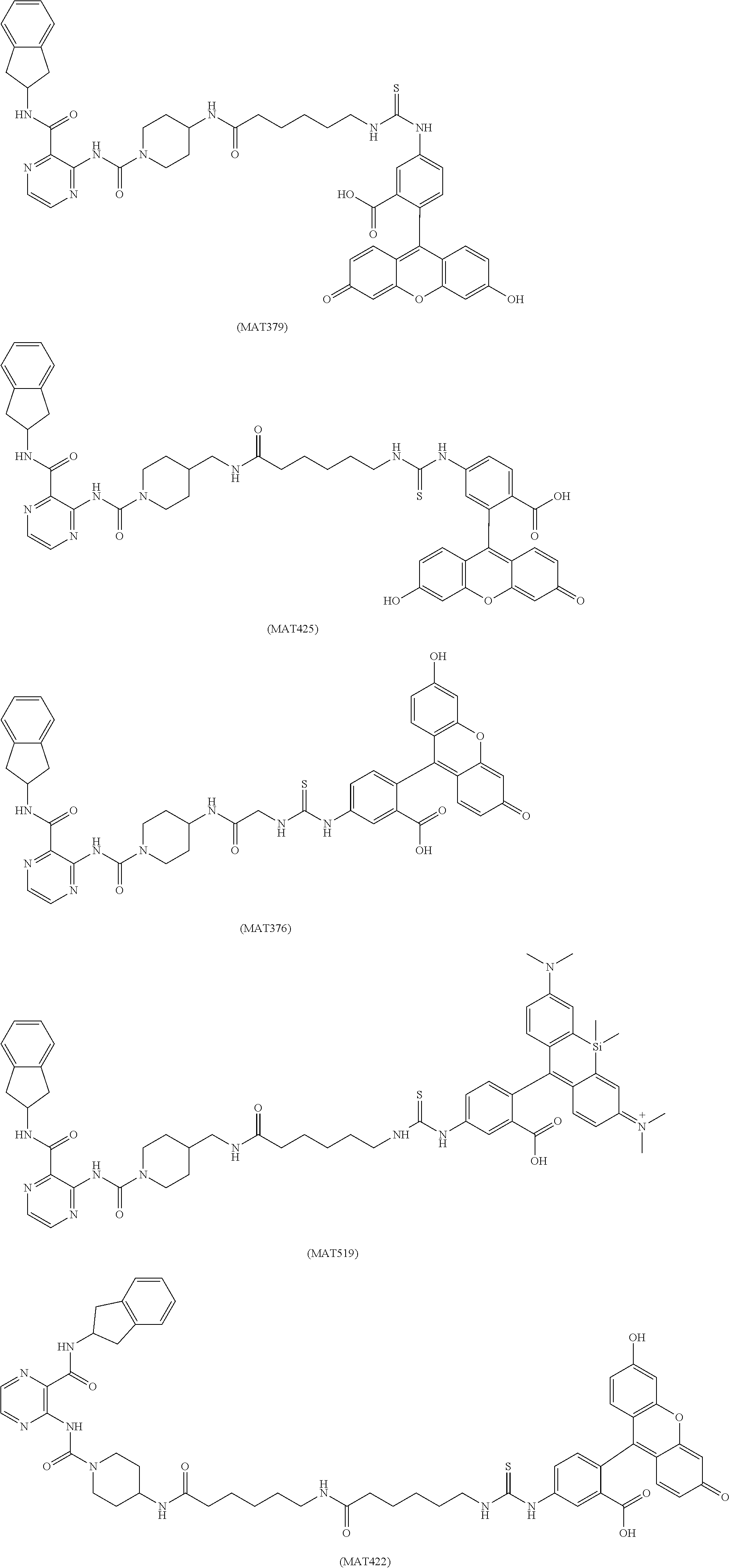

or a pharmaceutically acceptable salt thereof.

In some embodiments, the present disclosure provides a compound of Formula (III):

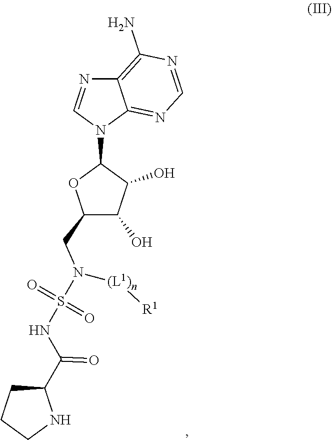

or a pharmaceutically acceptable salt thereof, wherein:
each L¹ is independently selected from O, S, S(═O)₂, NH, C═O, C═S, and C₁₋₆ alkylene;
n is an integer from 1 to 12; and
R¹ is a fluorophore.

In some embodiments, each L¹ is independently selected from O, NH, C═O, C═S, and C₁₋₆ alkylene.

In some embodiments, the moiety (L¹)ₙ comprises (C═O)O.

In some embodiments, the moiety (L¹)ₙ comprises NH(C═O)O.

In some embodiments, the moiety (L¹)ₙ comprises (C═O)—C₁₋₆ alkylene.

In some embodiments, the moiety (L¹)ₙ comprises NH(C═S)NH.

In some embodiments, the moiety (L¹)ₙ comprises NH(C═O).

In some embodiments, the moiety (L¹)ₙ comprises NH(C═O) NH.

In some embodiments, the moiety (L¹)ₙ comprises OCH₂CH₂O.

In some embodiments, the moiety (L¹)ₙ comprises OCH₂CH₂NH.

In some embodiments, R¹ is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.

In some embodiments, the compound has formula:

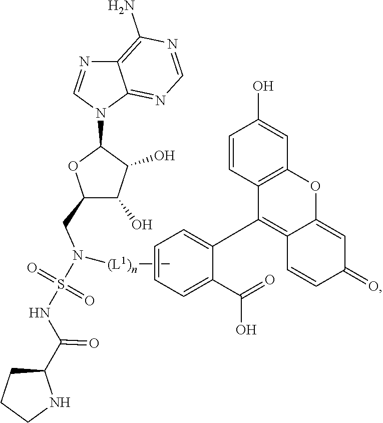

or a pharmaceutically acceptable salt thereof.

In some embodiments, the compound has formula:

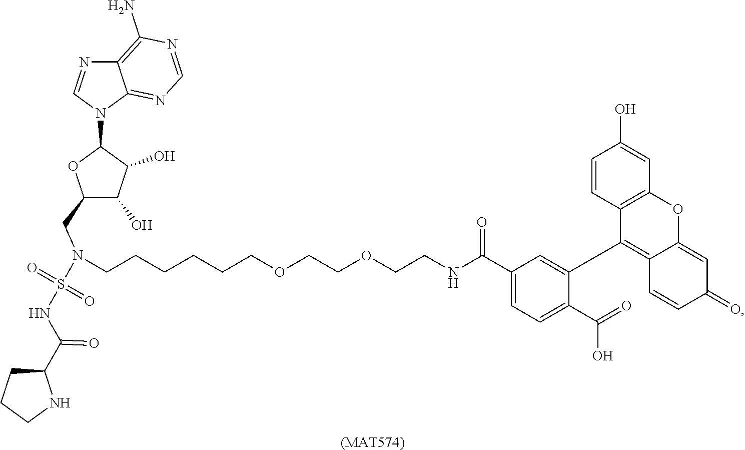 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H]2CCCN2)[C@@H](O)[C@H]1O ; "(MAT574)"]

or a pharmaceutically acceptable salt thereof.

In some embodiments, the present disclosure provides a compound of Formula (IV):

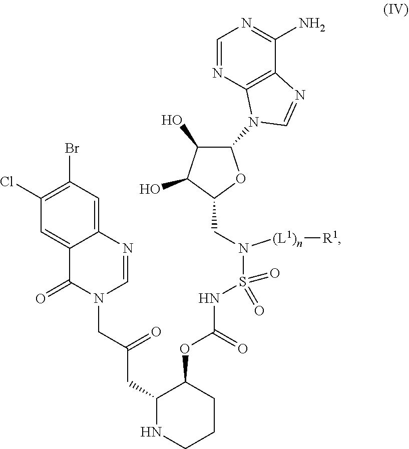

or a pharmaceutically acceptable salt thereof, wherein:
each L¹ is independently selected from O, S, S(═O)₂, NH, C═O, C═S, and C₁₋₆ alkylene;
n is an integer from 1 to 12; and
R¹ is a fluorophore.

In some embodiments, each L¹ is independently selected from O, NH, C═O, C═S, and C₁₋₆ alkylene.

In some embodiments, the moiety (L¹)ₙ comprises (C═O)O.

In some embodiments, the moiety (L¹)ₙ comprises NH(C═O)O.

In some embodiments, the moiety (L¹)ₙ comprises (C═O)—C₁₋₆ alkylene.

In some embodiments, the moiety (L¹)ₙ comprises NH(C═S)NH.

In some embodiments, the moiety (L¹)ₙ comprises NH(C═O).

In some embodiments, the moiety (L¹)ₙ comprises NH(C═O) NH.

In some embodiments, the moiety (L¹)ₙ comprises OCH₂CH₂O.

In some embodiments, the moiety (L¹)ₙ comprises OCH₂CH₂NH.

In some embodiments, the compound has formula:

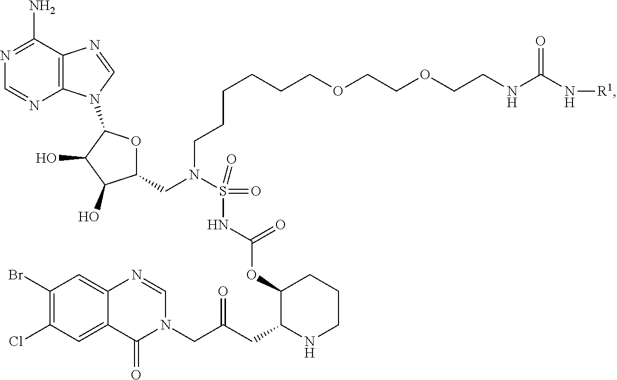

or a pharmaceutically acceptable salt thereof.

In some embodiments, R¹ is a FRET acceptor fluorophore.

In some embodiments, R¹ is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.

In some embodiments, the compound bas formula:

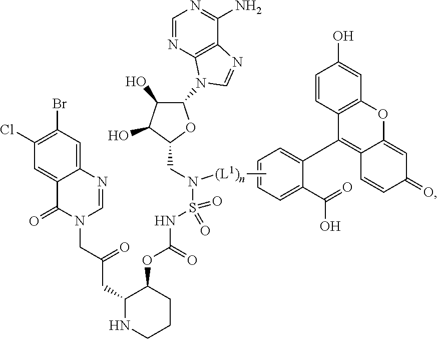

or a pharmaceutically acceptable salt thereof.

In some embodiments, the compound has formula:

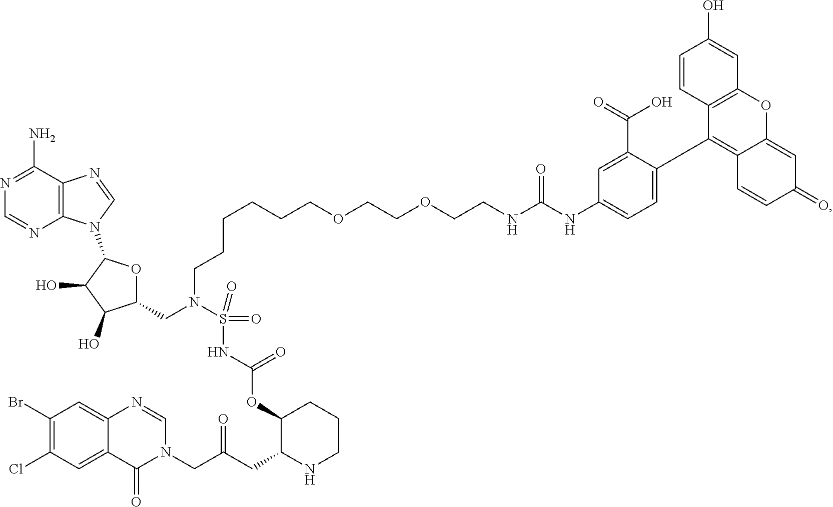 [SMILES: Nc1ncnc2c1ncn2[C@@H]1O[C@H](CN(CCCCCCOCCOCCNC(=O)Nc2ccc(-c3c4ccc(=O)cc-4oc4cc(O)ccc34)c(C(=O)O)c2)S(=O)(=O)NC(=O)O[C@H]2CCCN[C@@H]2CC(=O)Cn2cnc3cc(Br)c(Cl)cc3c2=O)[C@@H](O)[C@H]1O]

or a pharmaceutically acceptable salt thereof.

In some embodiments, the present disclosure provides a compound of Formula (V):

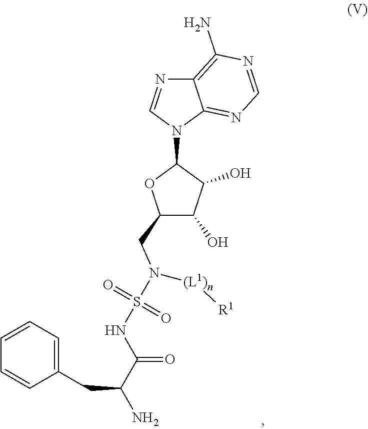

or a pharmaceutically acceptable salt thereof, wherein:
each L¹ is independently selected from O, S, S(═O)₂, NH, C═O, C═S, and C₁₋₆ alkylene;
n is an integer from 1 to 12; and
R¹ is a fluorophore.

In some embodiments, each L¹ is independently selected from O, NH, C═O, C═S, and C₁₋₆ alkylene.

In some embodiments, the moiety (L¹)ₙ comprises (C═O)O.

In some embodiments, the moiety (L¹)ₙ comprises NH(C═O)O.

In some embodiments, the moiety (L¹)ₙ comprises (C═O)—C₁₋₆ alkylene.

In some embodiments, the moiety (L¹)ₙ comprises NH(C═S)NH.

In some embodiments, the moiety (L¹)ₙ comprises NH(C═O).

In some embodiments, the moiety (L¹)ₙ comprises NH(C═O) NH.

In some embodiments, the moiety (L¹)ₙ comprises OCH₂CH₂O.

In some embodiments, the moiety (L¹)ₙ comprises OCH CH₂NH.

In some embodiments, the compound has formula:

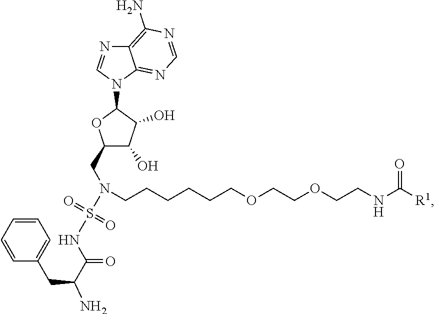

or a pharmaceutically acceptable salt thereof.

In some embodiments, R¹ is a FRET acceptor fluorophore.

In some embodiments, R¹ is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.

In some embodiments, the compound has formula:

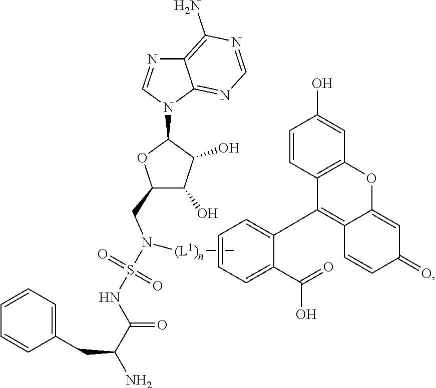

or a pharmaceutically acceptable salt thereof.

In some embodiments, the compound has formula:

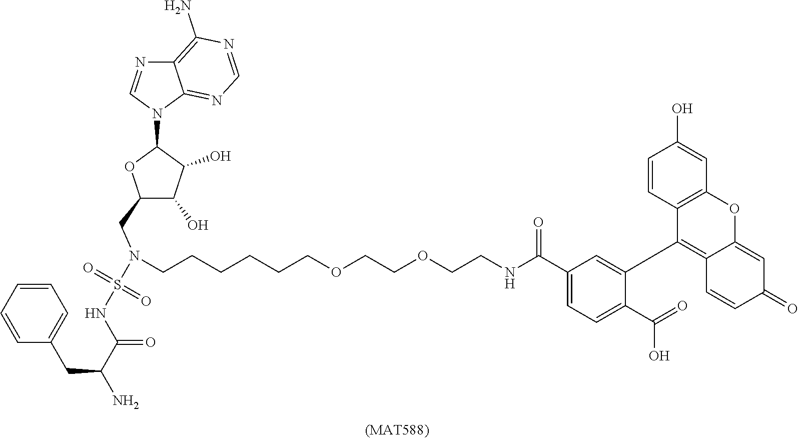 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H](N)Cc2ccccc2)[C@@H](O)[C@H]1O ; "(MAT588)"]

or a pharmaceutically acceptable salt thereof.

In some embodiments, the present disclosure provides a compound of Formula (VI):

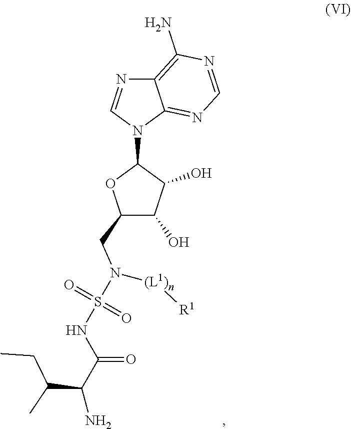

or a pharmaceutically acceptable salt thereof, wherein:
each L¹ is independently selected from O, S, S(═O)₂, NH, C═O, C═S, and C₁₋₆ alkylene;
n is an integer from 1 to 12; and
R¹ is a fluorophore.

In some embodiments, each L¹ is independently selected from O, NH, C═O, C═S, and C₁₋₆ alkylene.

In some embodiments, the moiety (L¹)ₙ comprises (C═O)O.

In some embodiments, the moiety (L¹)ₙ comprises NH(C═O)O.

In some embodiments, the moiety (L¹)ₙ comprises (C═O)—C₁₋₆ alkylene.

In some embodiments, the moiety (L¹)ₙ comprises NH(C═S)NH.

In some embodiments, the moiety (L¹)ₙ comprises NH(C═O).

In some embodiments, the moiety (L¹)ₙ comprises NH(C═O) NH.

In some embodiments, the moiety (L¹)ₙ comprises OCH₂CH₂O.

In some embodiments, the moiety (L¹)ₙ comprises OCH₂CH₂NH.

In some embodiments, the compound has formula:

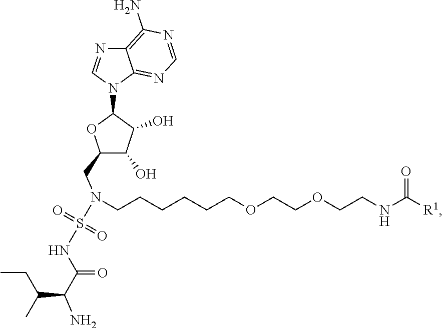

or a pharmaceutically acceptable salt thereof.

In some embodiments, R¹ is a FRET acceptor fluorophore.

In some embodiments, R¹ is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.

In some embodiments, the compound has formula:

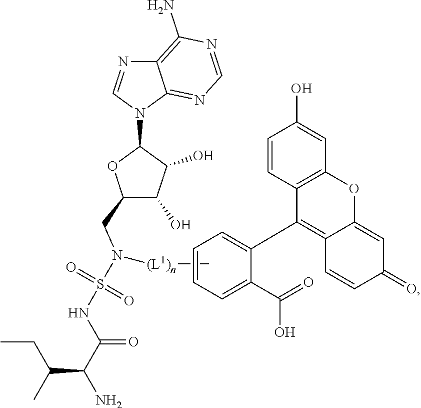

or a pharmaceutically acceptable salt thereof.

In some embodiments, the compound has formula:

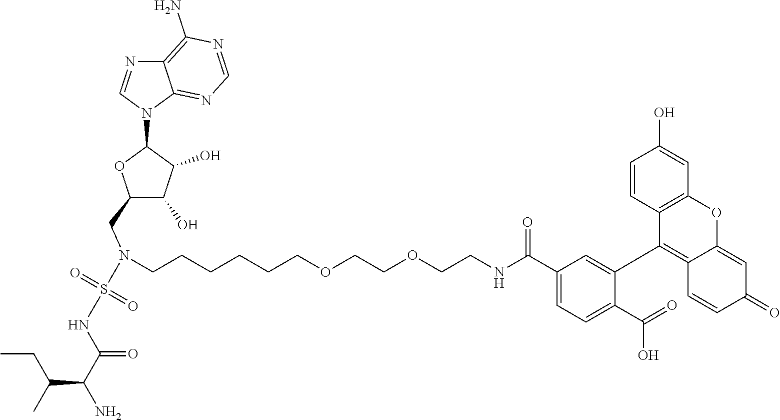 [SMILES: CCC(C)[C@H](N)C(=O)NS(=O)(=O)N(CCCCCCOCCOCCNC(=O)c1ccc(C(=O)O)c(-c2c3ccc(=O)cc-3oc3cc(O)ccc23)c1)C[C@H]1O[C@@H](n2cnc3c(N)ncnc32)[C@H](O)[C@@H]1O]

or a pharmaceutically acceptable salt thereof.

In some embodiments, the present disclosure provides a compound of Formula (VII):

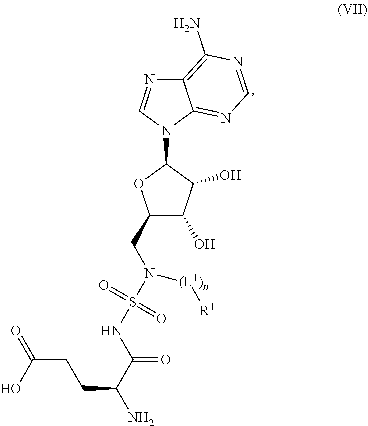

or a pharmaceutically acceptable salt thereof, wherein:
each L¹ is independently selected from O, S, S(═O)₂, NH, C═O, C═S, and C₁₋₆ alkylene;
n is an integer from 1 to 12; and
R¹ is a fluorophore.

In some embodiments, each L¹ is independently selected from O, NH, C═O, C═S, and C₁₋₆ alkylene.

In some embodiments, the moiety (L¹)ₙ comprises (C═O)O.

In some embodiments, the moiety (L¹)ₙ comprises NH(C═O)O.

In some embodiments, the moiety (L¹)ₙ comprises (C═O)—C₁₋₆ alkylene.

In some embodiments, the moiety (L¹)ₙ comprises NH(C═S)NH.

In some embodiments, the moiety (L¹)ₙ comprises NH(C═O)).

In some embodiments, the moiety (L¹)ₙ comprises NH(C═O) NH.

In some embodiments, the moiety (L¹)ₙ comprises OCH₂CH₂O.

In some embodiments, the moiety (L¹)ₙ comprises OCH CH₂NH.

In some embodiments, the compound has formula:

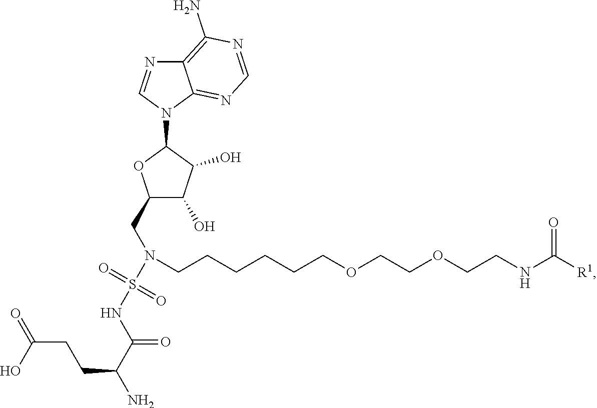

or a pharmaceutically acceptable salt thereof.

In some embodiments, R¹ is a FRET acceptor fluorophore.

In some embodiments, R¹ is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.

In some embodiments, the compound has formula:

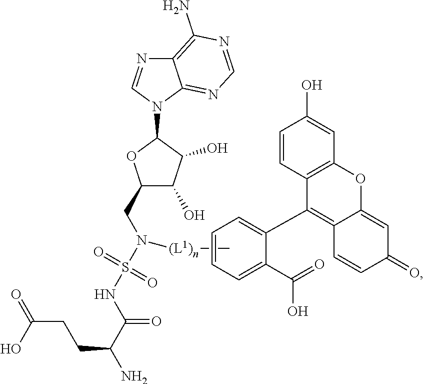

or a pharmaceutically acceptable salt thereof.

In some embodiments, the compound has formula

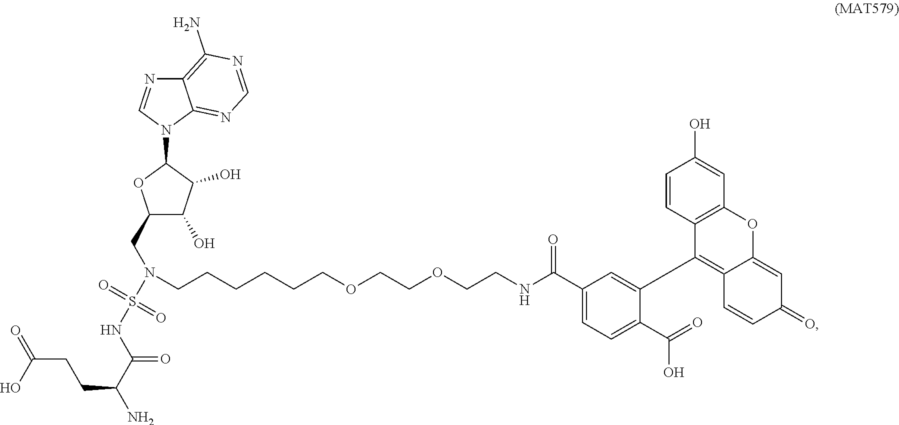 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H](N)CCC(=O)O)[C@@H](O)[C@H]1O]

or a pharmaceutically acceptable salt thereof.

In some embodiments, the present disclosure provides a composition comprising a tracer compound as described herein or a pharmaceutically acceptable salt thereof, and an inert carrier. For example, the insert carrier can be a pure water or a buffer solution (e.g., buffer with pH of from about 5 to about 8). The composition can be used, for example, in any of the assays described herein as a sample or a part of a sample where the protein of interest is being studied. In some embodiments, the composition is an aqueous solution. In some embodiments, the inert carrier is a buffer solution.

### Intermediate Compounds

Certain embodiments of synthetic intermediates to prepare some of the tracer compounds described above are provided in this disclosure. As such, the present disclosure provides a compound of Formula (A):

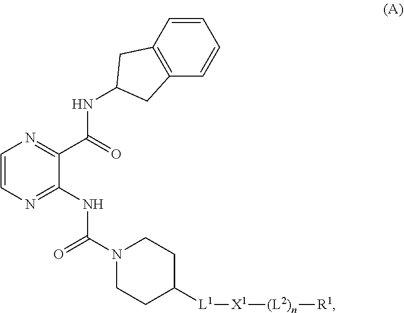

or a pharmaceutically acceptable salt thereof, wherein:
L¹ is C₁₋₃ alkylene, or L¹ is absent;
X¹ is selected from O and NR^{N};
R^{N} is selected from H, C₁₋₃ alkyl, and C₁₋₃ haloalkyl;
each L² is independently selected from O, S, S(═O)₂, NR^{N}, C═O, C═S, and C₁₋₆ alkylene;
n is an integer from 0 to 12; and
R¹ is selected from H, C₁₋₆ alkyl, and a protecting group.

In some embodiments, L¹ is absent.

In some embodiments, L¹ is C₁₋₃ alkylene.

In some embodiments, L¹ is selected from methylene, 1,2-ethylene, 1,1-ethylene, and propylene.

In some embodiments, L¹ is methylene.

In some embodiments, X¹ is O.

In some embodiments, X¹ is NH.

In some embodiments, the compound of Formula (A) has formula:

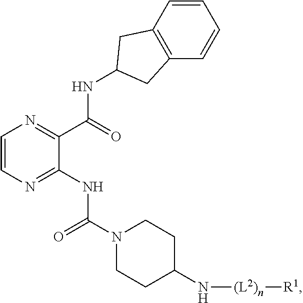

or a pharmaceutically acceptable salt thereof.

In some embodiments, the compound of Formula (A) has formula:

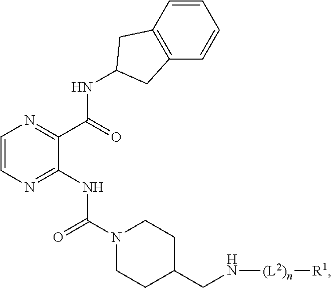

or a pharmaceutically acceptable salt thereof.

In some embodiments, n is 0.

In some embodiments, n is an integer from 1 to 12.

In some embodiments, each L² is independently selected from O, S, NH, C═O, C═S, and C₁₋₆ alkylene.

In some embodiments, the moiety (L²)ₙ comprises (C═O)O.

In some embodiments, the moiety (L²), comprises NH(C═O)O.

In some embodiments, the moiety (L²), comprises (C═O)—C₁₋₆ alkylene.

In some embodiments, the moiety (L²)ₙ comprises NH(C═O).

In some embodiments, the moiety (L²)ₙ comprises NH(C═O) NH.

In some embodiments, the moiety (L²)ₙ comprises NH(C═S)NH.

In some embodiments, the moiety (L²), comprises any one of the following fragments:

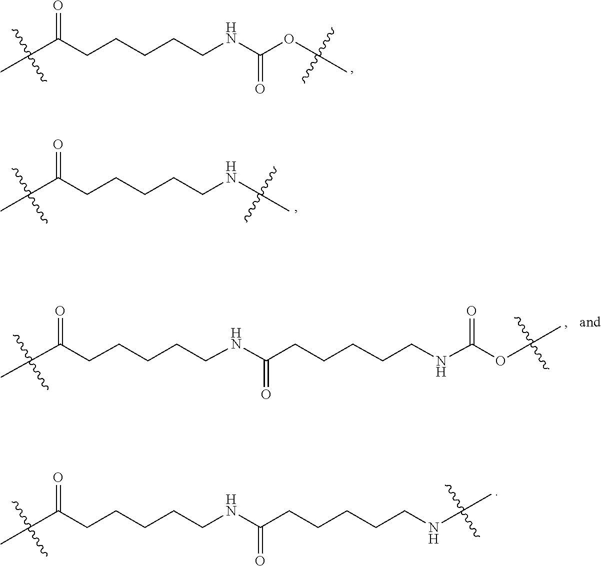 [SMILES: *OC(=O)NCCCCCC(*)=O ; *NCCCCCC(*)=O ; *OC(=O)NCCCCCC(=O)NCCCCCC(*)=O ; *NCCCCCC(=O)NCCCCCC(*)=O]

In some embodiments, R¹ is H.

In some embodiments, R¹ is C₁₋₆ alkyl.

In some embodiments, R¹ is an alcohol-protecting group, an amino-protecting group, or a carboxylic acid protecting group.

In some embodiments, the compound of Formula (A) is selected from any one of the following compounds:

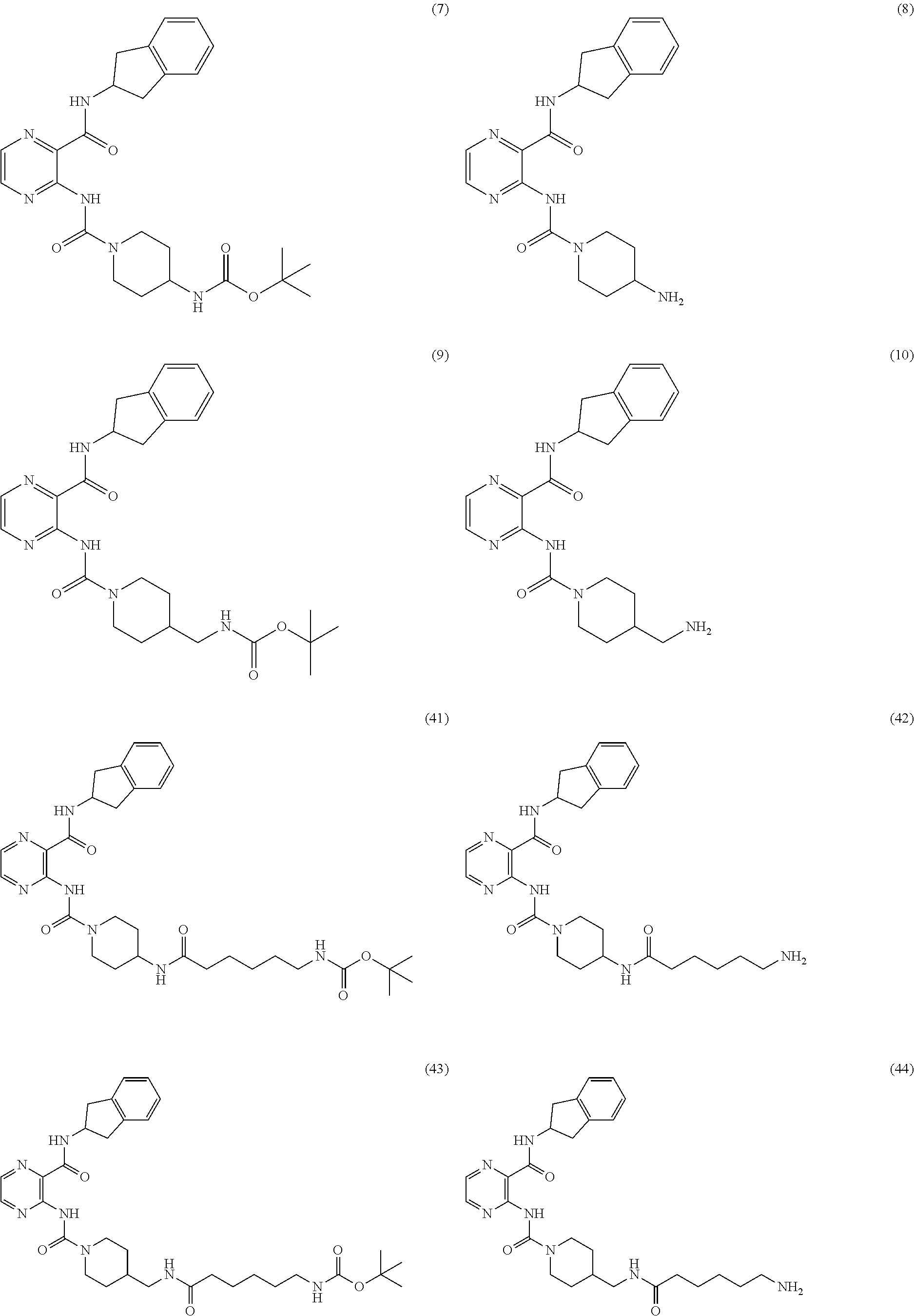

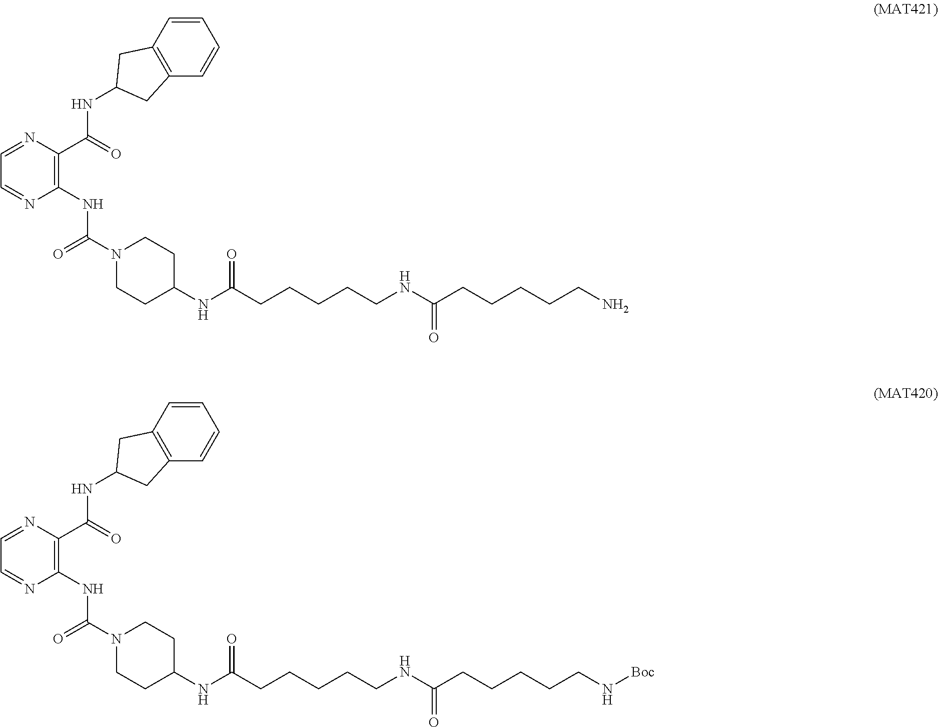 [SMILES: NCCCCCC(=O)NCCCCCC(=O)NC1CCN(C(=O)Nc2nccnc2C(=O)NC2Cc3ccccc3C2)CC1 ; CC(C)(C)OC(=O)NCCCCCC(=O)NCCCCCC(=O)NC1CCN(C(=O)Nc2nccnc2C(=O)NC2Cc3ccccc3C2)CC1]

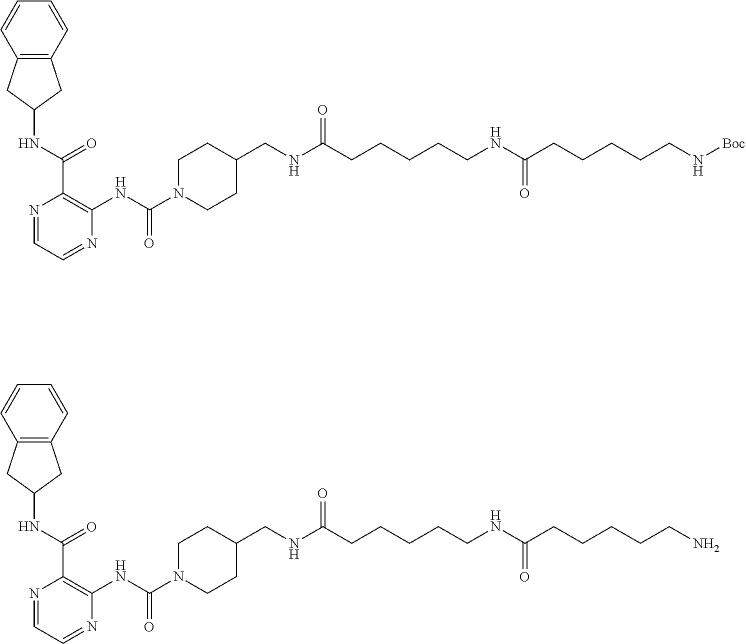 [SMILES: CC(C)(C)OC(=O)NCCCCCC(=O)NCCCCCC(=O)NCC1CCN(C(=O)Nc2nccnc2C(=O)NC2Cc3ccccc3C2)CC1 ; NCCCCCC(=O)NCCCCCC(=O)NCC1CCN(C(=O)Nc2nccnc2C(=O)NC2Cc3ccccc3C2)CC1]

or a pharmaceutically acceptable salt thereof.

### Therapeutic Compounds

In some embodiments, the present disclosure provides a compound of Formula (B):

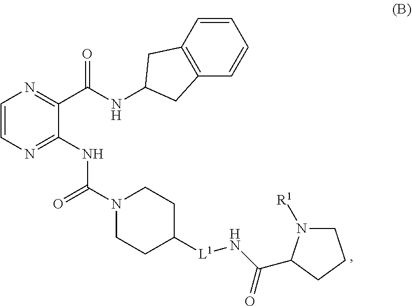

or a pharmaceutically acceptable salt thereof, wherein:
L¹ is C₁₋₃ alkylene; or L¹ is absent;
R¹ is selected from H, C₁₋₃ alkyl, C₁₋₃ haloalkyl, —C(═O)R^{A1}, and —C(═O)OR^{A1}; and
each R^{A1} is selected from H, C₁₋₆ alkyl, and C₁₋₃ haloalkyl.

In some embodiments, the compound has formula:

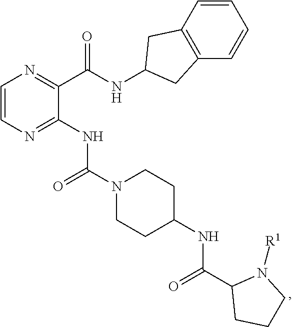

or a pharmaceutically acceptable salt thereof.

In some embodiments, L¹ is C₁₋₃ alkylene.

In some embodiments, the compound has formula:

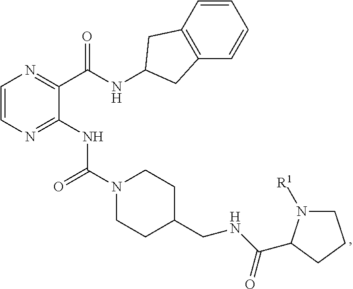

or a pharmaceutically acceptable salt thereof.

In some embodiments, R¹ is H.

In some embodiments, R¹ is C₁₋₃ alkyl.

In some embodiments, R¹ is —C(═O)R^{A1}.

In some embodiments, R¹ is —C(═O)OR^{A1}.

In some embodiments, R^{A1} is H. In some embodiments, R^{A1} is C₁₋₆ alkyl.

In some embodiments, the compound is selected from any one of the following compounds:

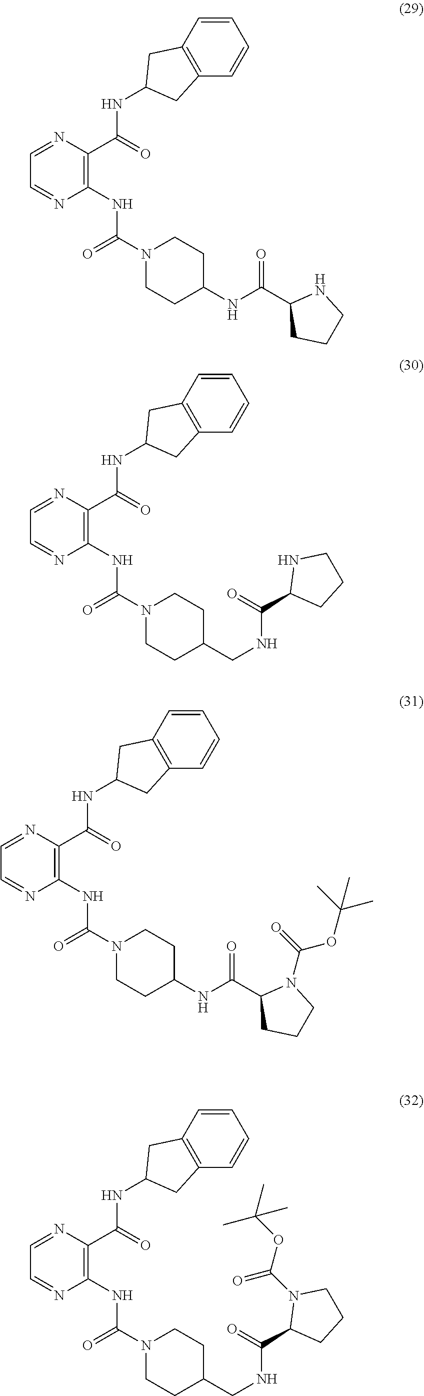 [SMILES: O=C(NC1Cc2ccccc2C1)c1nccnc1NC(=O)N1CCC(NC(=O)[C@@H]2CCCN2)CC1 ; O=C(NC1Cc2ccccc2C1)c1nccnc1NC(=O)N1CCC(CNC(=O)[C@@H]2CCCN2)CC1 ; CC(C)(C)OC(=O)N1CCC[C@H]1C(=O)NC1CCN(C(=O)Nc2nccnc2C(=O)NC2Cc3ccccc3C2)CC1 ; CC(C)(C)OC(=O)N1CCC[C@H]1C(=O)NCC1CCN(C(=O)Nc2nccnc2C(=O)NC2Cc3ccccc3C2)CC1]

or a pharmaceutically acceptable salt thereof.

### Pharmaceutically Acceptable Salts

In some embodiments, a salt of a compound is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate. acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, P-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH-(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. In some embodiments, the compounds, or pharmaceutically acceptable salts thereof, are substantially isolated.

### Methods of Treatment

Certain compounds of this disclosure may be useful for treating a disease or condition as described herein. These compounds include, for example, the compounds of Formula (B). The compounds of Formulae (I)-(VII) may be used as tracers (e.g., FRET acceptor-containing modulators of corresponding proteins of interest) that are useful in assays for diagnosing a disease or monitoring a treatment of a disease as described herein. Compounds of Formula (A) may be useful as synthetic intermediates for making these tracers for the assays.

Accordingly, the present disclosure also provides methods of inhibiting glutamyl-prolyl-tRNA synthetase, prolyl-tRNA synthetase, or a combination thereof. The inhibiting may be carried out in a cell, such as in vitro, in vivo, or ex vivo. In one example, the disclosure provides a method of inhibiting prolyl-tRNA-synthetase in a cell, comprising contacting the cell with a compound of this disclosure, or a pharmaceutically acceptable salt thereof. In some embodiments, the cell is a human cell or a protozoan parasitic cell. In some embodiments, the cell is a human cell (e.g., cancer cell). In some embodiments, the cell is a protozoan parasitic cell. In some embodiments, the protozoan parasitic cell is a Plasmodium parasitic cell. In some embodiments, the protozoan parasitic cell is a Plasmodium falciparum. In some embodiments, the protozoan parasitic cell is selected from the group consisting of a Cryptosporidium, Babesia, Cyclospora, Cystoisospora, Toxoplasma. Giardia. and Plasmodia parasitic cell. In some embodiments, the protozoan parasitic cell is selected a Plasmodia parasitic cell. In some embodiments, the protozoan parasitic cell is selected from the group consisting of Plasmodium vivax, Plasmodium falciparum, Plasmodium malariae, Plasmodium ovale, and Plasmodium knowlesi.

In some embodiments, the present disclosure provides a method of inhibiting prolyl-tRNA-synthetase (e.g., a glutamyl-prolyl-tRNA synthetase) in a subject, comprising administering to the subject an effective amount of a compound as described herein, or a pharmaceutically acceptable salt thereof.

In some embodiments, the human has been infected with protozoan parasite. In some embodiments, the human has been identified as having been infected with protozoan parasite. In some embodiments, the protozoan parasite is selected from the group consisting of Cryptosporidium, Babesia, Cyclospora, Cystoisospora, Toxoplasma, Giardia, and Plasmodium. In some embodiments, the human has been infected with a Plasmodium parasite. In some embodiments, the human has been identified as having been infected with a Plasmodium parasite. In some embodiments, the human has been identified as having been infected with a Plasmodium parasite (e.g., a drug resistant Plasmodium parasite) selected from the group consisting of Plasmodium vivax, Plasmodium falciparum, Plasmodium malariae, Plasmodium ovale, and Plasmodium knowlesi. In some embodiments, the human has been infected with Plasmodium falciparum. In some embodiments, the human has been identified as having been infected with Plasmodium falciparum. In some embodiments, the infected human is diagnosed with malaria.

The present application further provides methods of treating a disorder in a subject (e.g., a subject in need thereof). In some embodiments, the disorder is associated with (e.g., abnormal activity) glutamyl-prolyl-tRNA synthetase, prolyl-tRNA synthetase, or a combination thereof. The method typically includes administering to a subject a therapeutically effective amount of a compound of this disclosure, or a pharmaceutically acceptable salt thereof. In one example, the subject is in need of treatment, for example, the subject may be diagnosed with the disorder by a treating physician.

In some embodiments, the disorder is a parasitic infection. In some embodiments, the parasite is a protozoan parasite. In some embodiments, the parasite is a protozoan parasite selected from the group consisting of Cryptosporidium, Babesia, Cyclospora, Cystoisospora, Toxoplasma, Giardia, and Plasmodium. In some embodiments, the parasite is a Plasmodium parasite. In some embodiments, the parasite is a drug resistant parasite. In some embodiments, the parasite is a drug resistant Plasmodium parasite. In some embodiments, the parasite is Plasmodium falciparum. In some embodiments, the Plasmodium parasite (e.g., a drug resistant Plasmodium parasite) is selected from the group consisting of Plasmodium vivax, Plasmodium falciparum, Plasmodium malariae, Plasmodium ovale, and Plasmodium knowlesi. In some embodiments, the parasite is a drug resistant Plasmodium falciparum.

In some embodiments, the parasitic infection is selected from malaria, toxoplasmosis, leishmaniasis, cryptosporidiosis, coccidiosis, Chagas disease. African sleeping sickness, giardiasis, and babesiosis. In some embodiments, the disorder is malaria. In some embodiments, the infectious disease is malaria, wherein the malaria is associated with a Plasmodium parasite. In some embodiments, the infectious disease is malaria, wherein the malaria is associated with Plasmodium falciparum. In some embodiments, the Plasmodium falciparum is a drug resistant Plasmodium falciparum.

In some embodiments, the disorder is an autoimmune disease. In some embodiments, the autoimmune disease is selected from multiple sclerosis, rheumatoid arthritis, lupus, psoriasis. scleroderma, dry eye syndrome, Crohn's Disease, inflammatory bowel disease, chronic obstructive pulmonary disease (COPD), asthma, fibrosis, scar formation, ischemic damage, and graft versus host disease.

In some embodiments, the disorder is a bacterial infection. In some embodiments, the disorder is a fungal infection. In some embodiments, the disorder is a viral infection. In some embodiments, the viral infection caused by corona virus, dengue virus or chikungunya virus.

In some embodiments, the disorder is selected from neurological disorder (e.g., Alzheimer's, Parkinson's, Huntington's, or ALS), a genetic disorder, a cardiovascular disorder (e.g., ischemia, stroke), a protein aggregation disorder, a metabolic disorder, an inflammatory disorder, and a cosmetic disorder. Compounds of the present disclosure may also be used to promote wound healing and/or prevent scarring and may be useful cosmetically.

In some embodiments, the disorder is amino acid response (AAR)-mediated condition or a Th17-mediated condition. In certain embodiments, compounds of the present invention may be used to inhibit pro-fibrotic behavior in fibroblasts or inhibit the differentiation of Th 17 cells. Therefore, provided compounds may be useful in preventing fibrosis. Provided compounds may also be used as probes of biological pathways. Provided compounds may also be used in studying the differentiation of T cells.

In some embodiments, the genetic disorder is Duchenne muscular dystrophy. In some embodiments, the metabolic disorder is selected from diabetes and obesity. In some embodiments, the cosmetic disorder is selected from the group consisting of cellulite and stretch marks. In some embodiments, the inflammatory disorder is selected from restenosis, macular degeneration, choroidal neovascularization, and chronic inflammation. The disorder may also be a disorder involving angiogenesis, such as cancer. In some embodiments, the disorder is cancer. In some embodiments, the cancer is a T-cell neoplasm selected from mature T-cell leukemia, nodal peripheral T-cell lymphoma (PTCL), extranodal PTCLs, and cutaneous T-cell lymphoma (CTCL). In some embodiments, the cancer is selected from adrenocortical carcinoma, bladder urothelial carcinoma, breast invasive carcinoma, cervical squamous cell carcinoma and endocervical adenocarcinoma, cholangio carcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine corpus endometrial carcinoma, uterine carcinosarcoma, uveal melanoma, multiple myeloma, and chordoma.

The cancer may be any one of cancers described, for example, in Wang et al., Genes 2020, 11, 1384, and Arita et al., Biochemical and Biophysical Research Communications 488 (2017) 648-654, both of which are incorporated here by reference in their entirety.

### Compositions, Formulations, and Routes of Administration

The present application also provides pharmaceutical compositions comprising an effective amount of a compound of the present disclosure disclosed herein, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%. wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose. mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin. f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers. bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments. emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol. polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, a compound of the present disclosure is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly. twice weekly, once every two weeks, once a month).

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “C-m” indicates a range which includes the endpoints, wherein n and m arc integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl. 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. The term “alkylene” includes divalent alkyl groups.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl. butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(C.-m-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N. O. and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N=N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the aaRS with a compound of the invention includes the administration of a compound of the present invention to an individual or patient, such as a human, having aaRS, as well as, for example, introducing a compound of the invention into a sample containing a cellular or purified preparation containing the aaRS.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease, for example, ameliorating a disease, condition or disorder in an individual who is experiencing or displaying the pathology or symptomatology of the disease, condition or disorder (i.e., reversing the pathology and/or symptomatology).

### EXAMPLES

### Example 1—ProRS Assay

### Summary

The development of antimalarials that are efficacious against the human liver and asexual blood stages provide advantages for the treatment of the disease. Aminoacyl-tRNA synthetase (aaRS) enzymes, including prolyl-tRNA synthetase (ProRS), are targets for malaria chemotherapy. Described in this example is a novel single-step biochemical assay platform for Plasmodium (PfcProRS) and human ProRS (HsProRS) that overcomes critical limitations of existing technologies and enables quantitative ProRS inhibitor identification and profiling with unprecedented sensitivity and flexibility. The assay informs the inhibitor requirements, e.g., to overcome existing resistance mechanisms and therefore accelerates rational development of ProRS-targeted anti-malarial therapies.

### Introduction

Malaria is an infectious diseases caused by Plasmodium parasites and ranks third among deadly infectious diseases, with over 200 million cases and more than 600,000 deaths per year. The emergence and spread of resistance to first-line antimalarials threatens the ability to treat and contain malaria. This problem is exacerbated by the limited number of targets exploited by current drugs, most of which are only relevant for the asexual blood stage (ABS), restricting their utility to the treatment of acute malaria. Therefore, new antimalarial therapies that exploit novel targets and pathways essential for multiple life-cycle stages are highly sought after for primary prophylaxis and transmission blocking, in addition to acute treatment.

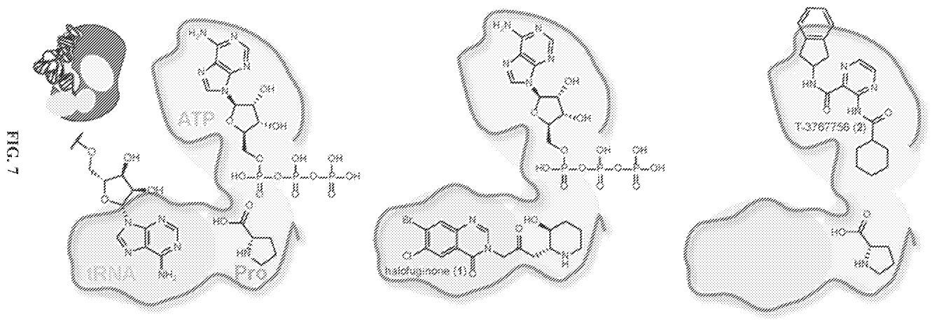

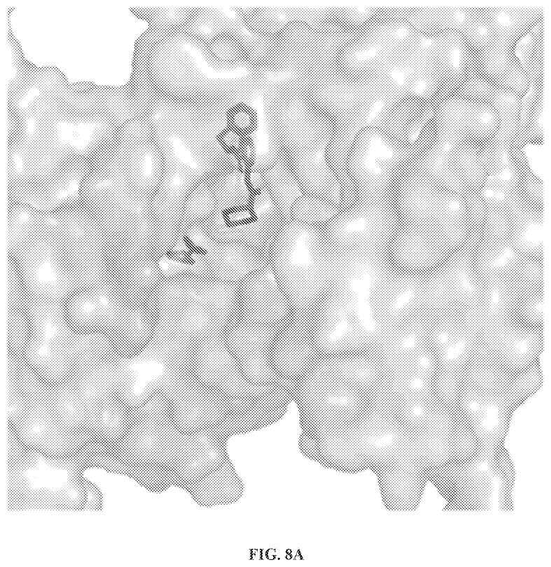

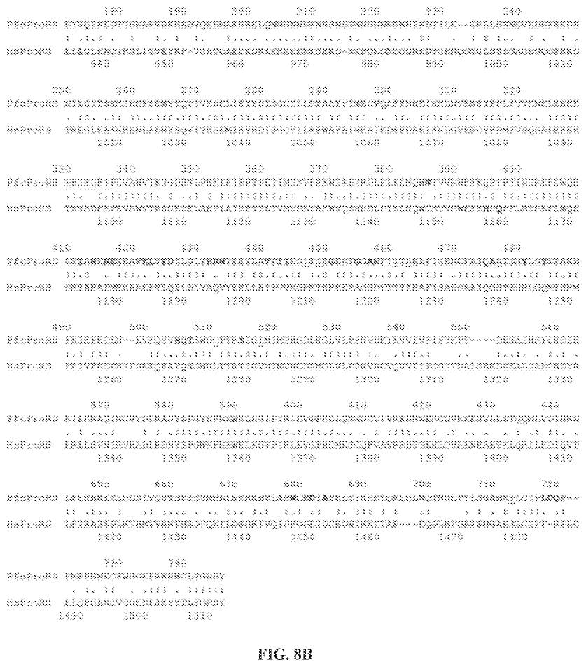

Halofuginone is one the most potent known antimalarials and a synthetic derivative of the natural product febrifugine (1), the curative ingredient of an ancient herbal remedy that has been used in Traditional Chinese Medicine for over 2,000 years for the treatment of fevers and malaria. However, the therapeutic utility of halofuginone and analogs as antimalarials has been stymied by poor tolerability, and the previously unknown mode of action in the host and parasite has impeded rational development of drugs with improved pharmacological properties. Cytoplasmic prolyl-tRNA synthetase (ProRS) was identified as the molecular target of halofuginone in P. falciparum. ProRS is a member of the aaRS enzyme family. which exist in all living cells and catalyze the transfer of amino acids to their cognate tRNAs. However, recent research has also revealed secondary functions of specific aaRS isoforms and tRNAs beyond their canonical role in protein biosynthesis. Halofuginone and derivatives are also active against liver stage parasites in vitro and in vivo, further validating ProRS as an attractive target for antimalarial drug development. In complementary efforts, investigating the mode of action of halofuginone in humans, where halofuginone has been studied as chemotherapeutic, antifibrotic, immunomodulatory agent and more recently as antiviral drug, prolyl-tRNA synthetase activity of the bifunctional glutamyl-prolyl-tRNA synthetase (HsGluProRS) was identified as the mechanistic target. Crystallographic data of the co-complexes with human and Plasmodium ProRS revealed that halofuginone binds the A76-tRNA^{Pro} and proline-binding pockets of the active site (FIG. 7), which are highly conserved between both homologs. Despite the high homology between parasite and host enzymes (see FIG. 8) and comparable biochemical potency, halofuginone is significantly more active against asexual blood-stage P. falciparum than mammalian cell lines. As to the evolution of halofuginone resistance, within five generations (10 days) bulk cultures, termed halofuginone-induced parasites, mounted a 10-20-fold tolerance by upregulation (˜20-fold) of intracellular proline, which is competitive with halofuginone. This previously unrecognized mode of resistance could potentially also explain the failure of febrifugine and halofuginone to control recrudescence in vivo and their narrow therapeutic indices as antimalarials. While these findings have validated ProRS as a well-defined target for the rational development of next-generation antimalarials with multi-stage activity, the development of rapid resistance to halofuginone analogs has raised concerns. Furthermore, a general problem that has plagued aaRS inhibitor discovery is the lack of robust, simple yet sensitive high-throughput assays that are suitable for supporting rational drug development. Although several biochemical assay platforms for aaRS isoforms have been reported, including assays for ProRS and ProRS, they collectively suffer from several shortcomings that greatly limit sensitivity, robustness, and throughput. Particularly, the high enzyme concentrations that are required for these assays renders them incapable of accurately profiling potent inhibitors. Non-radioactive aaRS assays generally require 0.1-0.5 μM enzyme and are consequently incapable of accurately measuring K_{D}-values substantially below this concentration range. Additionally. current assay platforms require long incubation times and multiple manipulation steps that increase variability, largely preclude the measurement of binding kinetics, and are generally challenging to implement in high-throughput screening (HTS) settings. The assay within the present claims is a straightforward, single-step biochemical assay that facilitates HTS and reliable ligand characterization, including kinetic and substrate-dependent profiling, and therefore greatly accelerates inhibitor development for this enzyme family.

### Results—TR-FRET ProRS Assay Development

Rational drug development efforts depend on researchers' ability to reliably characterize the biochemical activity, including the substrate-dependent mode of inhibition for PfcProRS and HsProRS. Current aaRS assay platforms suffer from several inherent limitations that render the accurate determination of binding affinities, binding kinetics, and mode of inhibition difficult. These constraints are particularly relevant for potent ligands, which existing assays cannot differentiate or quantitatively measure their affinity values.

Time-resolved Förster resonance energy transfer (TR-FRET) assays possesses favorable characteristics including high sensitivity, specificity, and flexibility, and offer an equally straightforward and robust platform for the quantitative characterization of aaRS ligands (FIG. 2a, 2B, 2C). However, no suitable fluorescently-labeled tracers for TR-FRET (or analogous fluorescence polarization) assays have been reported for ProRS or other aaRS enzymes. This is likely because the aaRS active site is generally deeply buried in the ligand-bound state, which renders the development of linker-modified ligands challenging.

### Development of ProRS-Specific Tracers

The active site of ProRS comprises three distinct pockets that bind ATP, proline, and the 3′-terminal adenosine residue of tRNA^{Pro} (A76), respectively (FIG. 1a). A class of HsProRS inhibitors is represented by T-3767758 (2) (FIG. 1a). Unlike halofuginone and analogs, which span the A76 and proline-binding sites and interact in an ATP-uncompetitive manner (i.e., the inhibitor affinity increases with increasing ATP concentration), this inhibitor class targets the ATP-binding pocket and features adjacent to the active site. Notably T-3767758 (2) (FIG. 1a) displayed proline-uncompetitive steady state kinetics for HsProRS. This property is desirable for halofuginone-tolerant strains with elevated intracellular proline (halofuginone-induced), as it allows to overcome or even select against this resistance mechanism. Sequence analysis of HsProRS and PfcProRS paralogs reveals overall high homology with several nonconserved residues in and adjacent to the binding site occupied by compound 2, suggesting that inhibitors containing its structural core are active against PfcProRS (FIG. 8). On the basis of compound 2, a ProRS inhibitor (7) with nanomolar potency was identified:

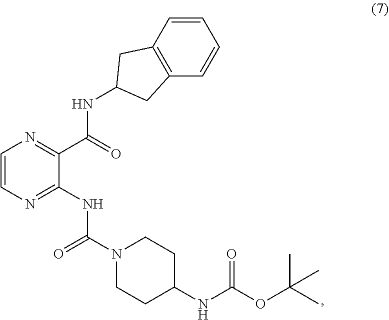 [SMILES: CC(C)(C)OC(=O)NC1CCN(C(=O)Nc2nccnc2C(=O)NC2Cc3ccccc3C2)CC1 ; ","]

Biochemical and in vitro activity of compound 7:

| | Dd2 | | | | | | | | |
|---|---|---|---|---|---|---|---|---|---|
| | (EC₅₀ [nM]) | | | PfcProRS | | | HsProRS | | |
| | | Halofuginone- | | (K_{D} [nM]) | | | (K_{D} [nM]) | | |
| Compound | wt | induced | HFGR-I | − | +proline | +ATP | − | +proline | +ATP |
| 7 | 90.6 | 2,170 | 2,750 | 13.1 | 13.2 | — | 22.1 | 34.8 | — |

4-amino-piperidyl substituent of ProRS inhibitor compound 7 identified above represents a suitable position for linker functionalization, providing fluorescent tracers for TR-FRET-based ligand displacement assays (FIG. 2a, 2B, 2C). Replacement of the BOC-group with an acyl linker follows the triphosphate exit vector, as in the halofuginone-ATP PfcProRS co-crystal structure (e.g. PDB: 40LF). On this basis. TR-FRET tracers were prepared, such as MAT379 (24) and MAT425, shown below, that were appropriate for the development of a single-step ligand displacement assay, enabling screening of active site inhibitors for ProRS:

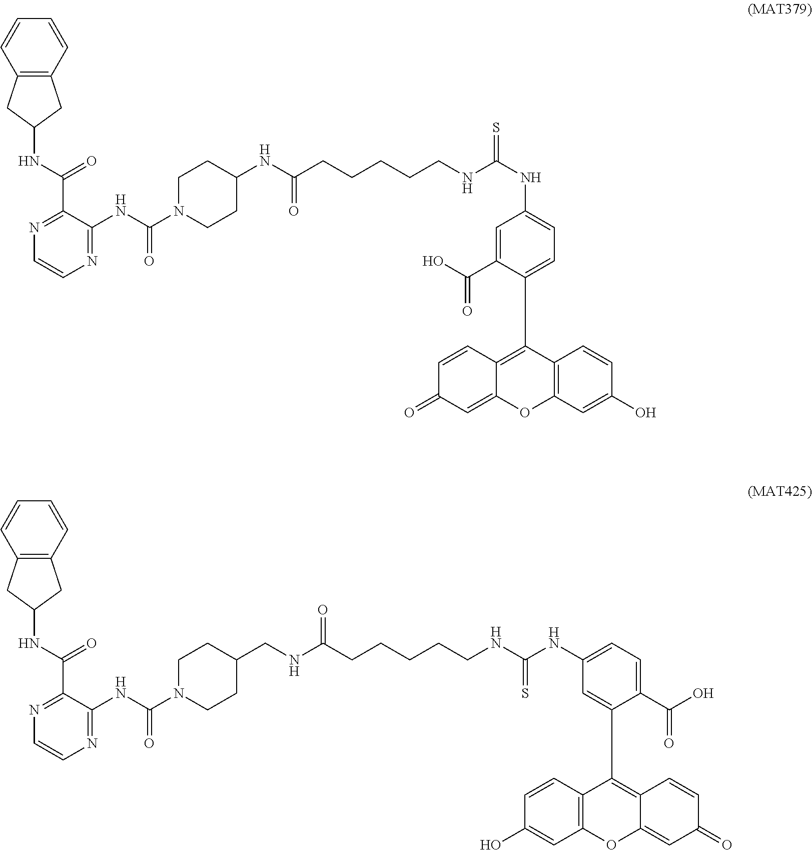 [SMILES: O=C(CCCCCNC(=S)Nc1ccc(-c2c3ccc(=O)cc-3oc3cc(O)ccc23)c(C(=O)O)c1)NC1CCN(C(=O)Nc2nccnc2C(=O)NC2Cc3ccccc3C2)CC1 ; O=C(CCCCCNC(=S)Nc1ccc(C(=O)O)c(-c2c3ccc(=O)cc-3oc3cc(O)ccc23)c1)NCC1CCN(C(=O)Nc2nccnc2C(=O)NC2Cc3ccccc3C2)CC1]

A codon-optimized construct was cloned and the prolyl-tRNA synthetase domain of PfcProRS (aa249-746) was recombinantly expressed as an N-terminal His6-HaloTag fusion protein (HT-PfcProRS). The HaloTag is a self-labeling protein tag that allows for efficient and defined covalent attachment of HaloTag-ligand modified small molecules, which were exploited to functionalize HT-PfcProRS with CoraFluor-1-Halo as the TR-FRET donor.

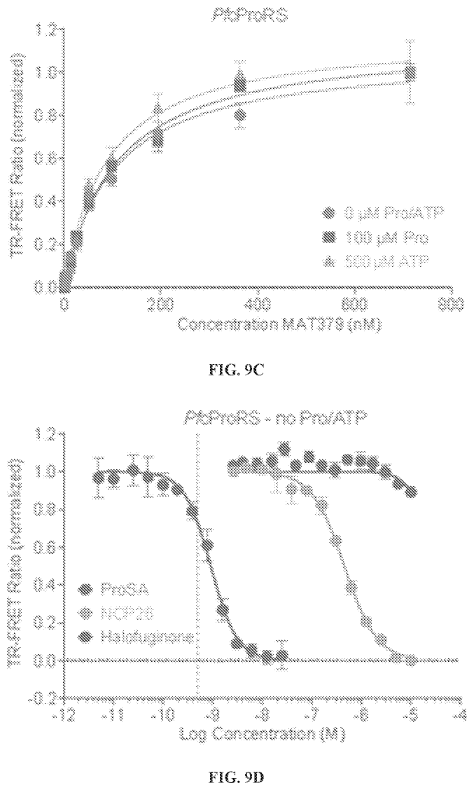

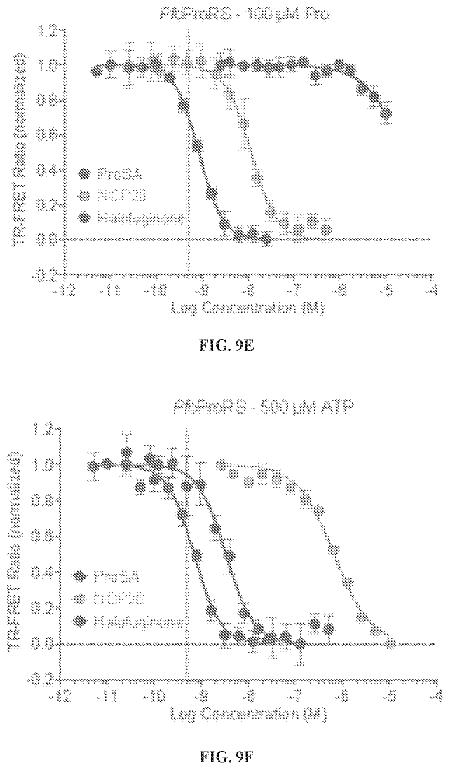

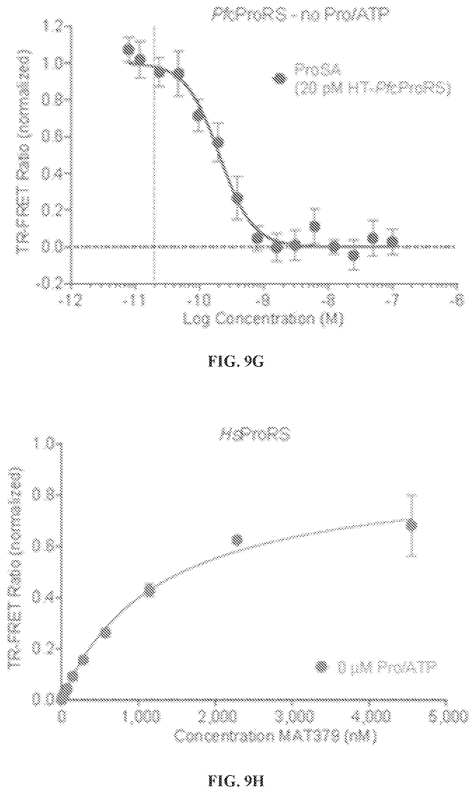

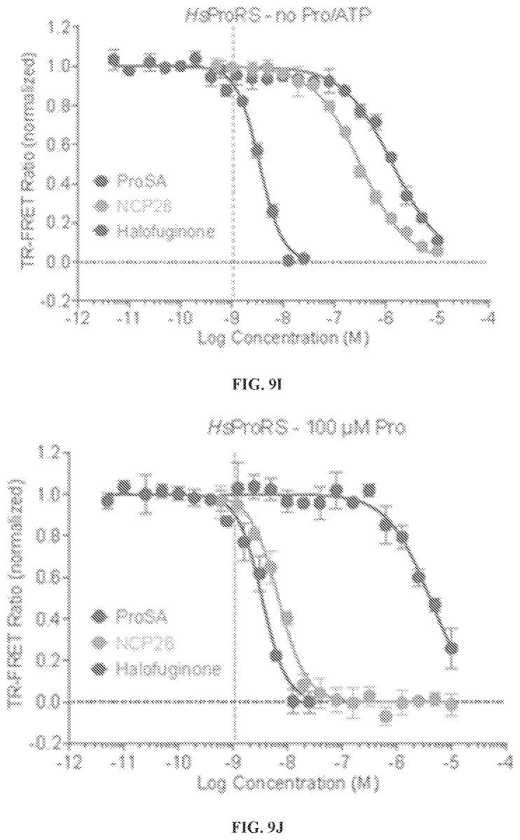

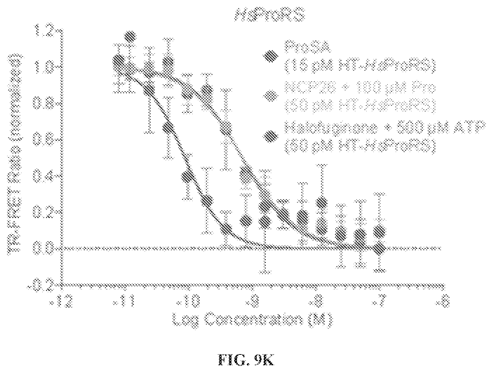

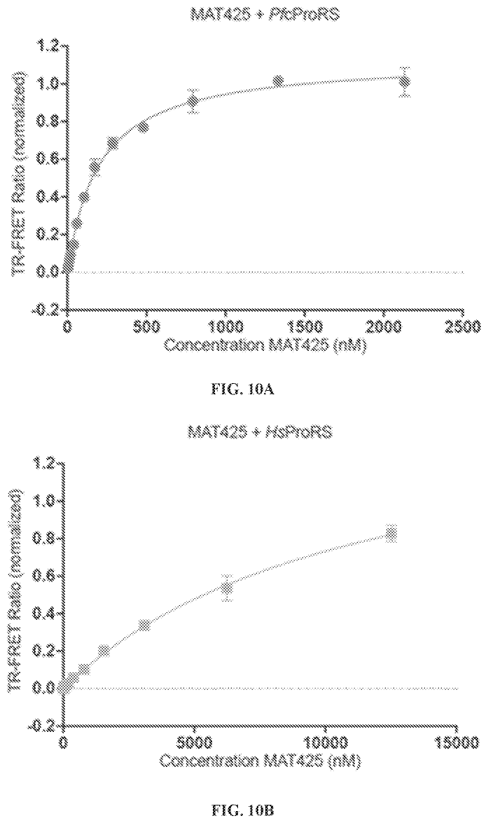

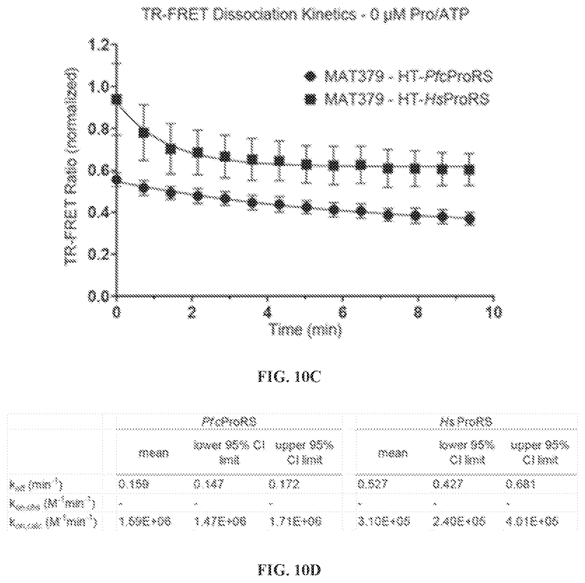

Saturation binding studies with CoraFluor-1-labeled HT-PfcProRS (1 nM) and MAT379 or MAT425 showed a dose-dependent increase in TR-FRET signal consistent with a specific, one-site, monophasic association model (FIG. 9c, 10). Non-linear regression analysis yielded an equilibrium dissociation constants (K_{D}) of 100 nM and 199 nM, respectively. MAT379 was selected for further characterization because of its higher affinity. To determine the minimum incubation time required for equilibrium conditions (five half-lives), the first-order dissociation rate constant (koff) was measured for MAT379 by 10-fold dilution of an equilibrated solution of CoraFluor-1-labeled HT-PfcProRS (100 nM) and ˜EC₈₀ MAT379 (560 nM) which yielded k_{off}-value of <0.16 min⁻¹ (FIG. 10c-d), suggesting that quasi-equilibrium is reached within 15 min (unless the test compounds themselves exhibit slow binding kinetics).

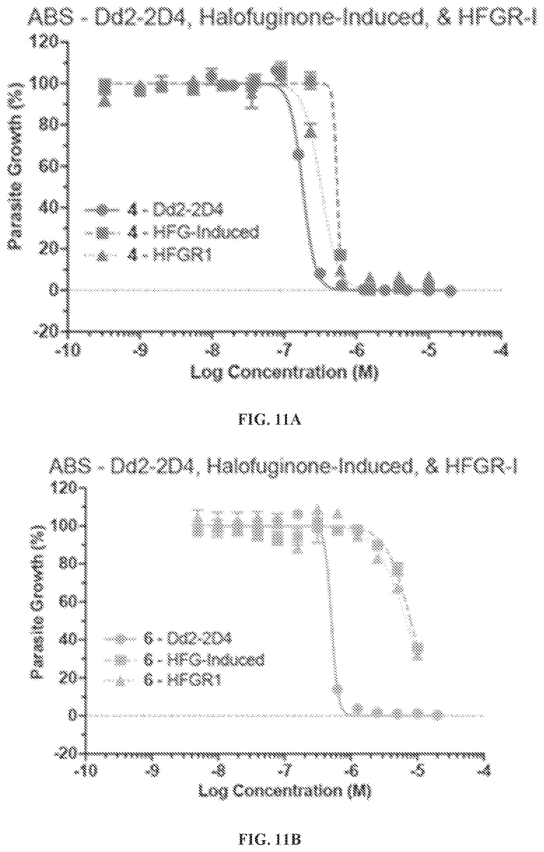

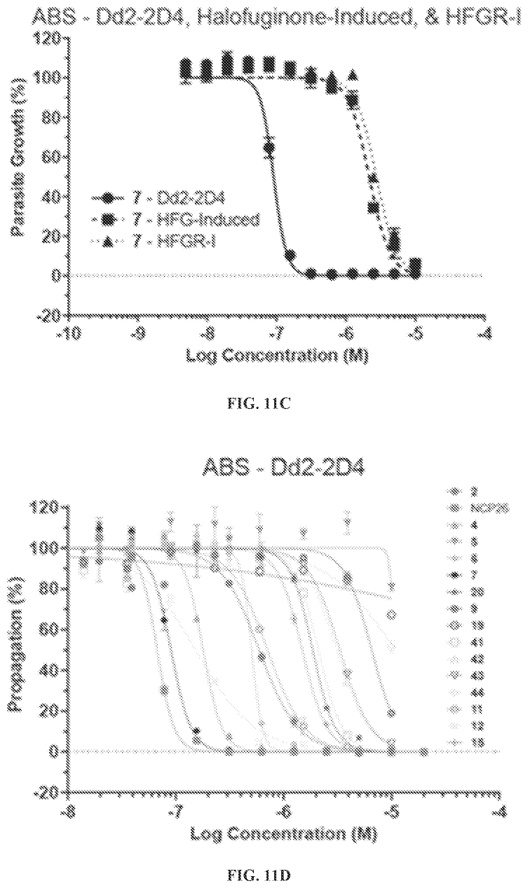

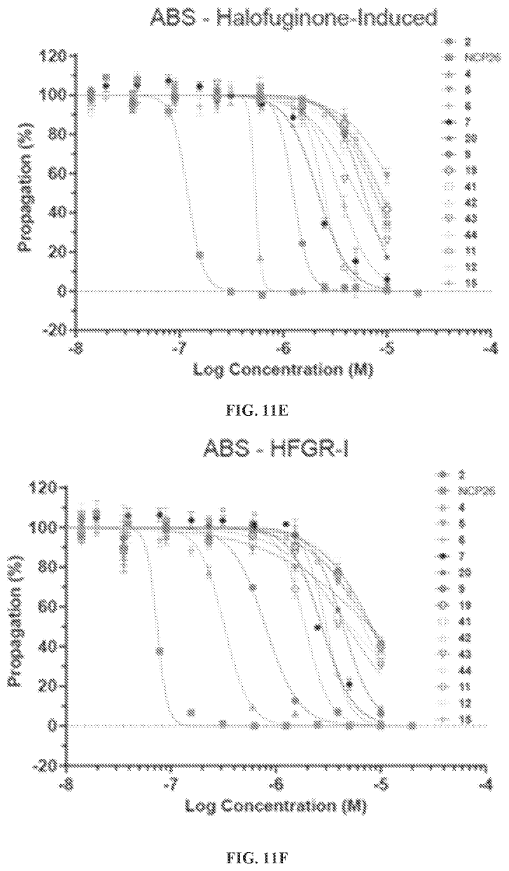

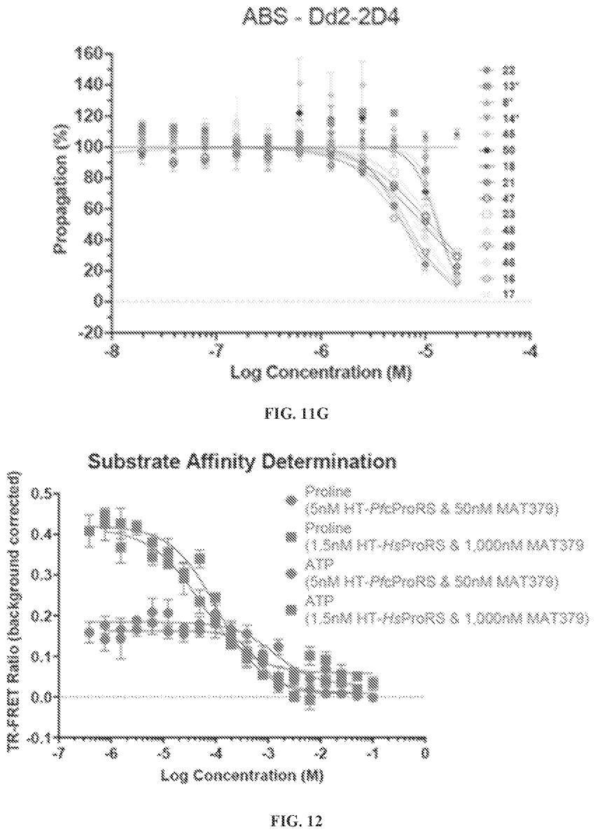

Dose-titration of ATP and proline revealed that MAT379 is not only ATP-competitive, as predicted herein, but, unlike compound 2, also proline-competitive, which is consistent with the finding that compound 7 was less active in halofuginone-induced and HFGR-1 parasites (FIG. 11). Quantification of the individual binding affinities yielded K_{D}-values for ATP (K_{D}=892 μM) and proline (K_{D}=457 μM) (FIG. 12). This outcome is ideal since it allows for highly sensitive, direct determination of both substrate-dependence and inhibitor-competitiveness with respect to proline and ATP, offering a unique advantage over other assay platforms.

To further explore the scope and limitations of the TR-FRET assay approach, next was used a reference compound set comprising halofuginone (proline-competitive and ATP-uncompetitive), NCP26 (proline-uncompetitive and ATP-competitive), and the non-hydrolyzable prolyl-AMP analog ProSA (25) (proline- and ATP-competitive) Dose-response titration of the test compounds were performed using 250 nM MAT379 as tracer (2.5×K_{D}) in the absence or presence of 100 μM proline or 500 μM ATP. As shown in FIG. 9d-f, NCP26 exhibited a 52-fold increased affinity in the presence of proline, consistent with the predicted herein proline-uncompetitive mode of inhibition (K_{D,0 μM proline}=130 nM vs K_{D,0 μM proline}=2.52 nM). By contrast, halofuginone displayed ATP-uncompetitive binding, showing a >6,000-fold increased affinity in the presence of ATP (K_{D,0 μM ATP}>3 μM vs K_{D,500 μM ATP}=503 μM). However, the exceptionally high affinity of ProSA resulted in titrating the enzyme (K_{D}<0.5 nM; [HT-PfcProRS]=1 nM) and limited the ability to obtain accurate equilibrium dissociation constants under these conditions.

To further improve the sensitivity of the assay for high-affinity ligands, was explored an alternative labeling strategy employing a CoraFluor-1-labeled anti-His6 antibody, which enabled the installation of multiple TR-FRET donors in addition to the donor covalently attached to the HaloTag label. This approach allowed to perform the assay with as little as 20 μM HT-PfcProRS, which is >1,000 and 100-fold lower than current non-radioactive and radioactive assay platforms, respectively. his strategy enabled the determination of an accurate K_{D}-value for ProSA (K_{D}=55.9 μM) (FIG. 9g).

Although the direct labeling of HT-PfcProRS with CoraFluor-1-Halo was less sensitive than the CoraFluor-1-labeled antibody-based strategy, it was still sufficient for most applications and was therefore selected as the default platform because of its simplicity and excellent robustness (Z′>0.95 at 500 μM HT-PfcProRS, Z′=0.71 at 250 μM HT-PfcProRS, and Z′=0.60 at 20 μM HT-PfcProRS supplemented with I nM CoraFluor-1-labeled anti-His6 antibody).

Following the same approach, the assay was adapted and optimized for HsProRS to enable quantitative comparative biochemical profiling and, as an added benefit, facilitate discovery efforts aiming to target the human paralog directly. As for PfcProRS, HsProRS (aa996-1512) was expressed as an N-terminal His6-HaloTag fusion protein (HT-HsProRS), which was readily labeled with CoraFluor-1-Halo. Dose-titration of MAT379 and MAT425 revealed that the respective tracers bound to HT-HsProRS 17-fold less tightly (K_{D,MAT379}=1.70 μM) and >50-fold less tightly (K_{D,MAT425}>10 μM) relative to HT-PfcProRS, establishing MAT379 as the preferred tracer for both ProRS paralogs (FIG. 9h, Supplementary FIG. 10b). Consistent with reduced affinity of MAT379 for HT-HsProRS compared to HT-PfcProRS, the measured first-order dissociation rate was faste

Although MAT379 exhibited lower affinity for HT-HsProRS, the tracer was still well suited for assay development. The slight decrease in signal intensity was readily compensated by increased enzyme concentration to yield an equally robust assay (Z′=0.80 at 1 nM CoraFluor-1-labeled HT-HsProRS and Z′=0.76 at 50 μM CoraFluor-1-labeled HT-HsProRS supplemented with 1 nM CoraFluor-1-labeled anti-His6 antibody). Similar to HT-PfcProRS, it was found that MAT379 binding to HT-HsProRS to be both ATP (K_{D}=30.6 M) and proline (K_{D}=67.1 μM) competitive. Next were determined the K_{D}-values of the reference inhibitor set in the presence and absence of substrates for HsProRS (FIG. 9i-k). Interestingly, NCP26 was marginally selective for PfcProRS in the absence of substrates but displayed >750-fold increased affinity for HsProRS in the presence of 100 μM proline (compared to 52-fold for PfcProRS), yielding a K_{D}-value of 351 μM for the host enzyme, which is comparable to the affinity of halofuginone in the presence of 500 μM ATP (K_{D}=225 μM).

### Exemplary Profiling of Selected Inhibitors

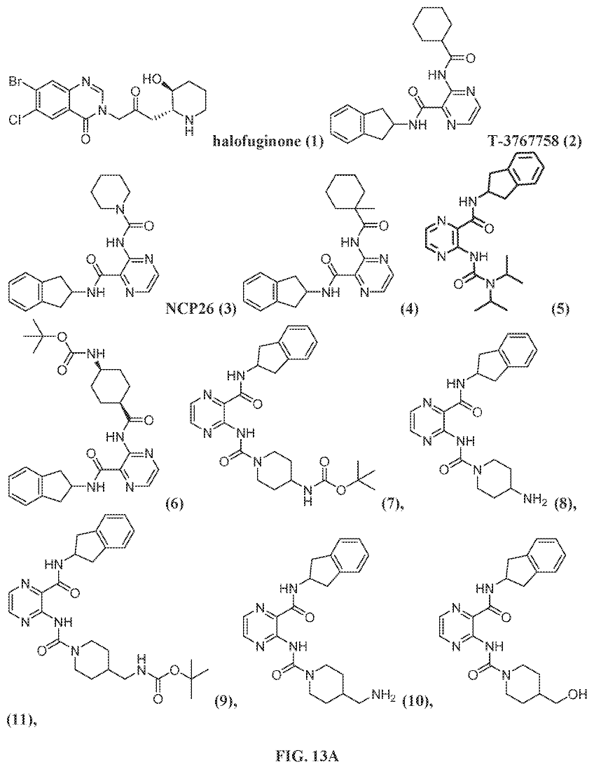

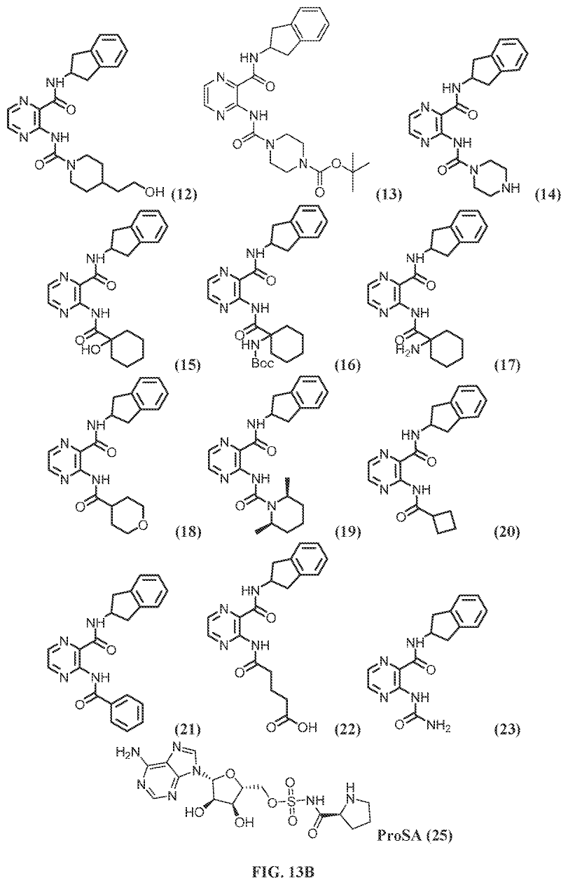

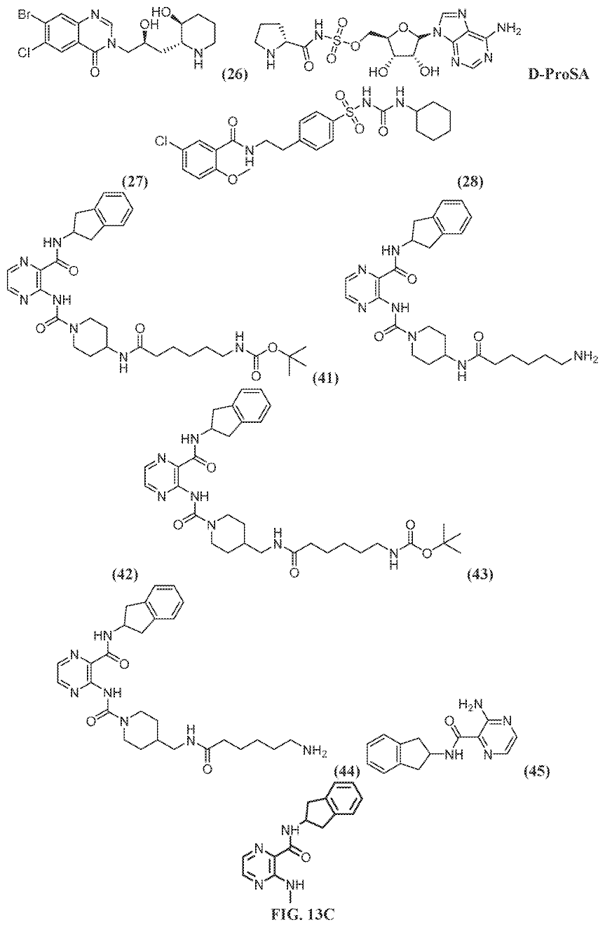

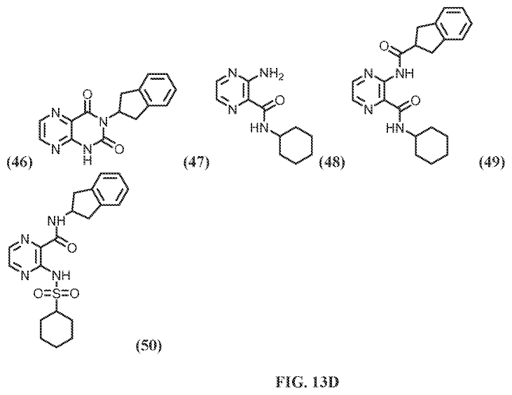

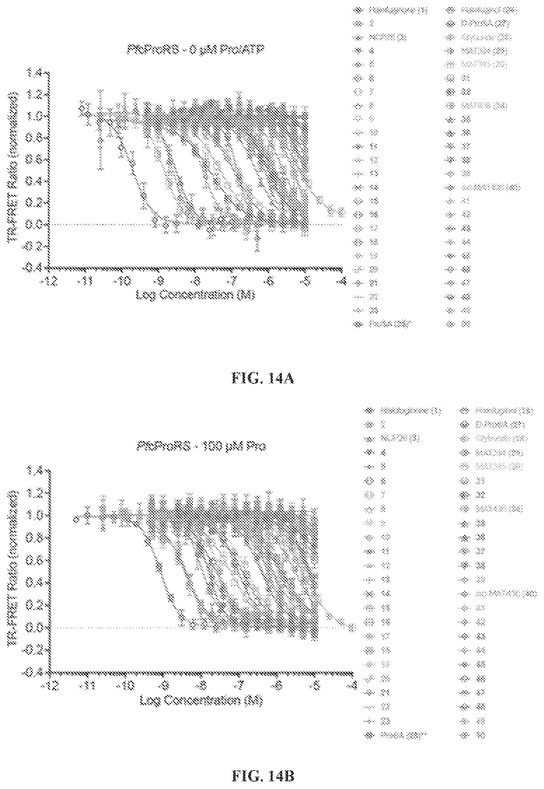

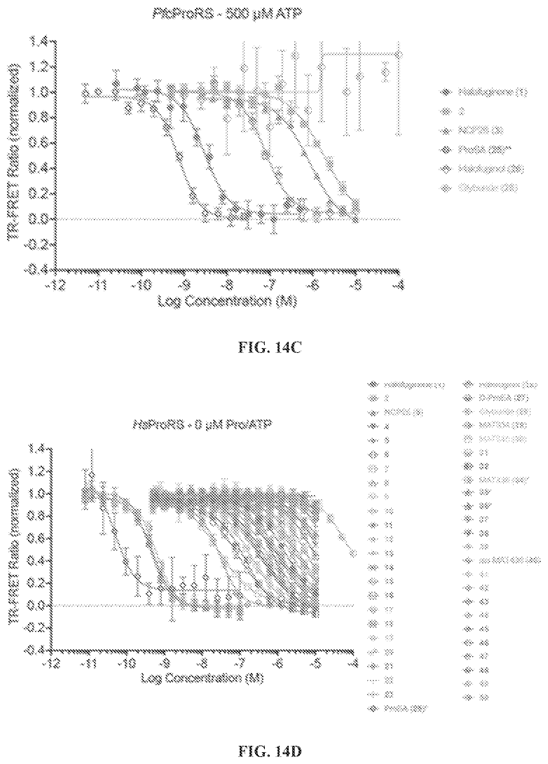

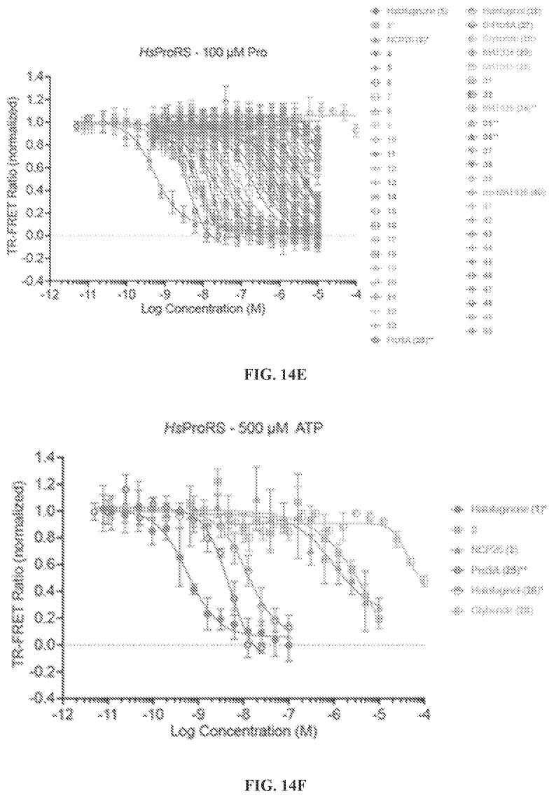

Following the comprehensive validation of the TR-FRET assay platform, the complete set of pyrazinamide compounds was profiled (see FIG. 13A-D) together with several reference compounds such as halofuginol (26), D-ProSA (27) as a negative control for ProSA, and glyburide (28). The inhibitor set was first tested in a dose-response format against both CoraFluor-1-Halo-labeled HT-PfcProRS and HT-HsProRS, in the absence and presence of individual substrates, to determine the quantitative binding affinities and modes of inhibition. Inhibitors that exhibited ligand depletion under the default assay conditions were retested at lower ProRS concentrations using the antibody-based labeling protocol (FIG. 14).

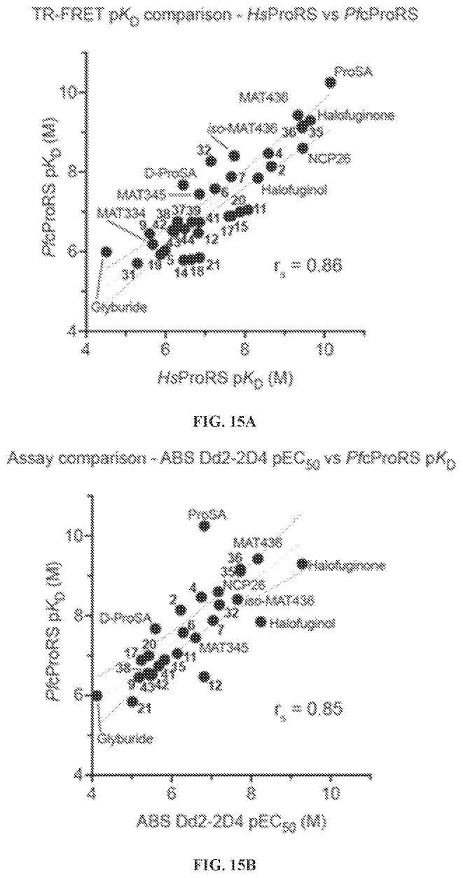

Most pyrazinamides were unselective or displayed modest preference for HsProRS (FIG. 15). Overall, the relative biochemical affinity of the pyrazinamide series tracked well with the cell-based activity, with proline-uncompetitive inhibitors showing little to no differential activity in wild-type, halofuginone-induced, and HFGR-I parasite strains, while proline-competitive compounds exhibited substantial cross-resistance, consistent with on-target activity (Table 1, and FIG. 15).

It was also confirmed that glyburide, which has previously been identified as a parasite-selective inhibitor that targets PfcProRS allosterically adjacent to the active site displayed >30-fold selectivity in the absence of substrates, and, consistent with the original report, was ATP- and proline-competitive (FIG. 14, Table 1).

### Validation of Pyrazinamide Mode of Action

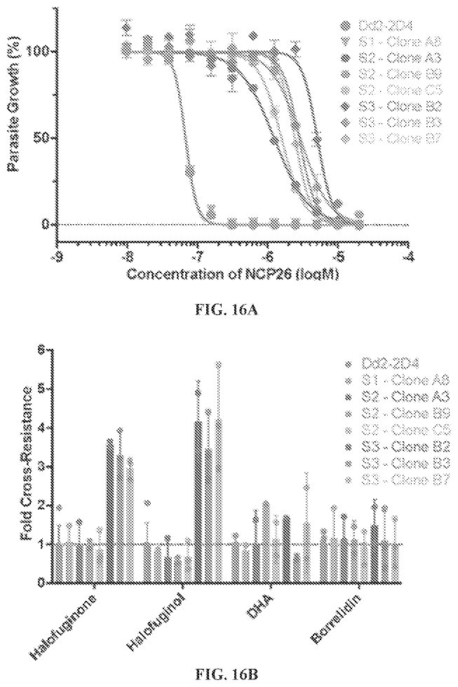

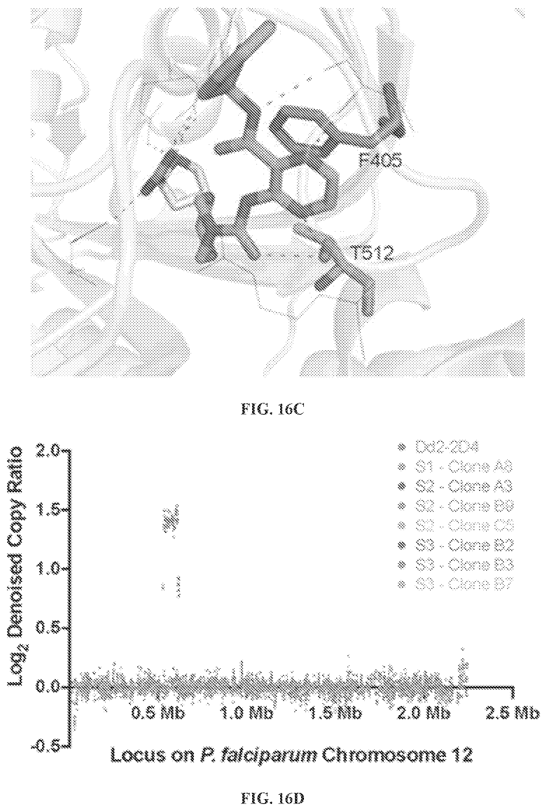

The strong correlation (rₛ=0.85) between the biochemical activity of the inhibitor set and the cellular potency against asexual blood stage P. falciparum showed that the antiparasitic activity of pyrazinamide analogs is the direct consequence of on-target activity. To experimentally validate PfcProRS as the principle functional target and to further assess the propensity of resistance evolution, three independent selections (S1-3) were conducted under intermittent drug pressure with NCP26 in asexual blood stage P. falciparum Dd2-2D4 parasites in vitro. Unlike halofuginone, which yields resistant parasites within <5 generations, moderate resistance (˜20-to-80-fold) was observed under NCP26 drug-pressure only in large scale experiments (10⁹ parasites) and prolonged culture periods (>50 generations, 100 days) in 2 out of 3 selections. Parasites were then cloned from all three selections, including S1, which failed to yield resistant bulk parasites, as additional reference. Like the bulk populations, the clonal lines exhibited the same level of NCP26-resistance (FIG. 16a), but no (S1-2) or low-level (<5-fold, S3) cross-resistance to halofuginone analogs and no differential sensitivity to other drugs, such as dihydroartemisinin (DHA) or the threonyl-tRNA synthetase inhibitor borrelidin (FIG. 16b).

Whole genome analysis of the individual NCP26-resistant parasite clones identified two independent single amino acid changes, PfProRS^{F405L} in S2 and PfcProRS^{T512S} in S3, mapping to the adenosine binding pocket, which form direct contacts with the pyrazinamide core (FIG. 16c). Notably, while no copy number variation (CNV) was observed in parasites carrying the PfcProRS^{T512S} mutation, PfcProRS^{F405L} clones had a ˜3-fold amplification of the intra-chromosomal region harboring the PfcProRS locus, with one mutant and two wildtype alleles, which is also consistent with the slightly reduced activity of halofuginone toward these parasites (FIG. 16d). Together, the results establish strong evidence that PfcProRS constitutes the mechanistic target of NCP26.

### Selected ProRS Inhibitors

Compounds were prepared that arc inhibitors (e.g., dual-site inhibitors) of ProRS. Exemplary compounds are MAT334 (29) and MAT345 (30):

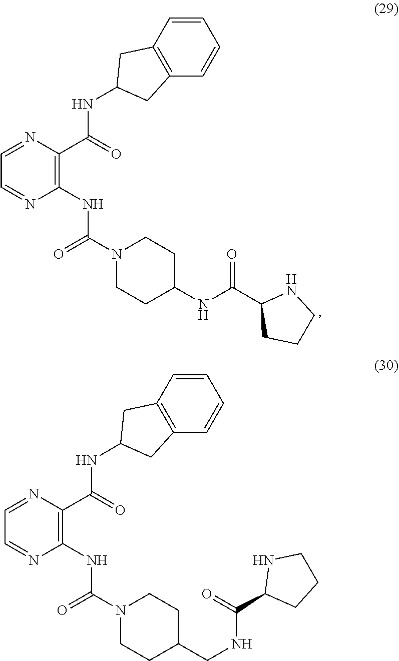 [SMILES: O=C(NC1Cc2ccccc2C1)c1nccnc1NC(=O)N1CCC(NC(=O)[C@@H]2CCCN2)CC1 ; "," ; O=C(NC1Cc2ccccc2C1)c1nccnc1NC(=O)N1CCC(CNC(=O)[C@@H]2CCCN2)CC1]

their BOC-protected precursors (31 and 32) were also prepared:

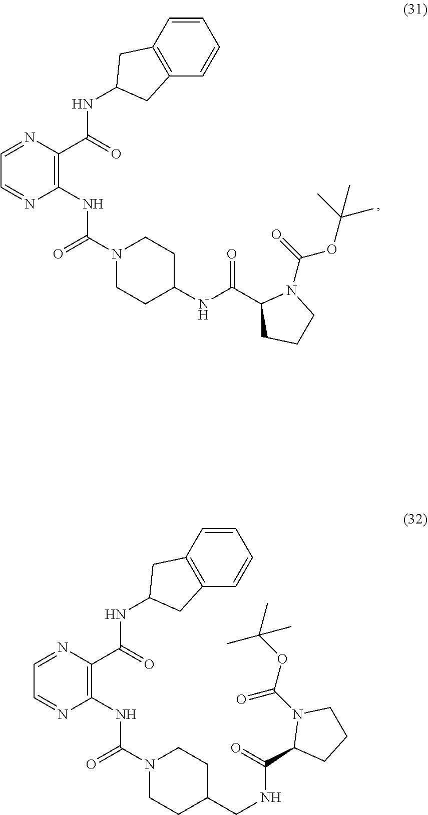 [SMILES: CC(C)(C)OC(=O)N1CCC[C@H]1C(=O)NC1CCN(C(=O)Nc2nccnc2C(=O)NC2Cc3ccccc3C2)CC1 ; "," ; CC(C)(C)OC(=O)N1CCC[C@H]1C(=O)NCC1CCN(C(=O)Nc2nccnc2C(=O)NC2Cc3ccccc3C2)CC1]

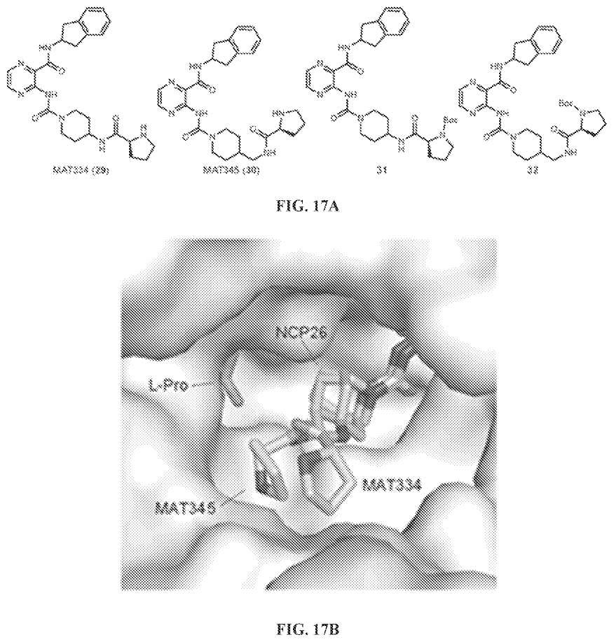

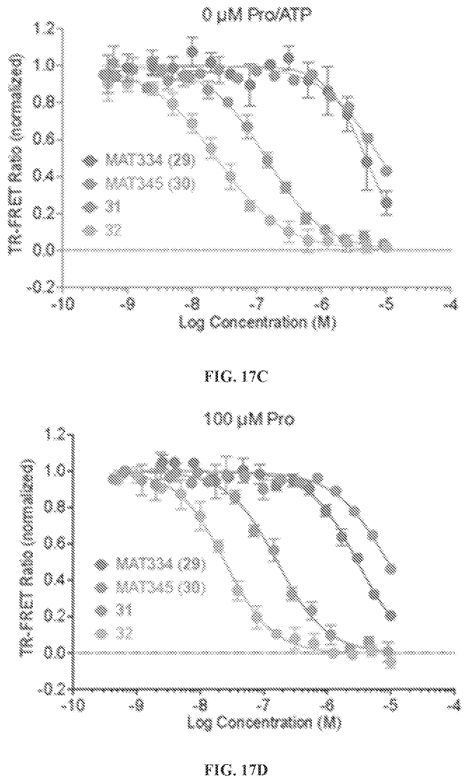

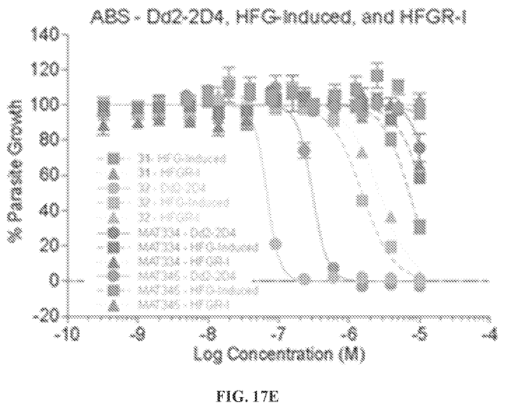

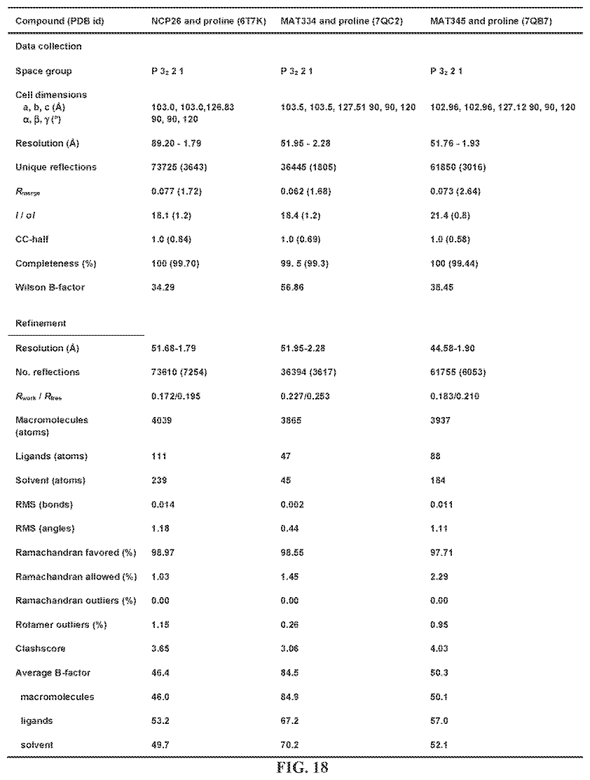

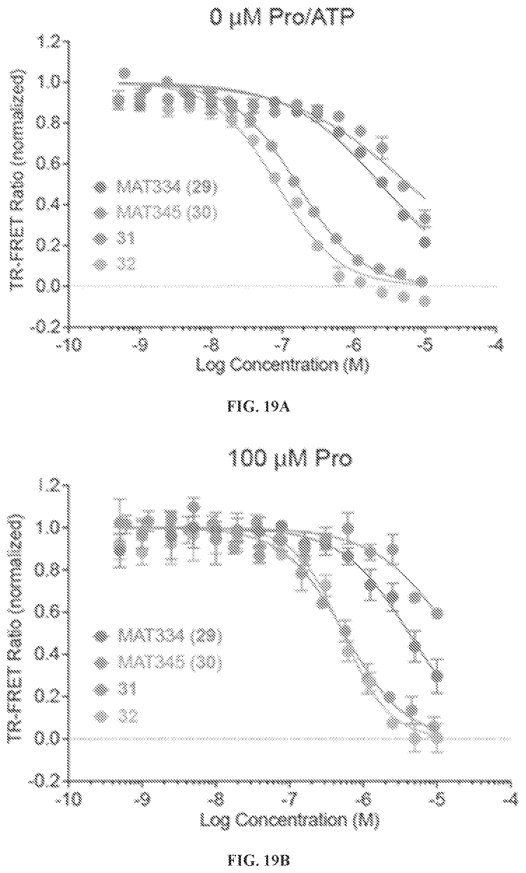

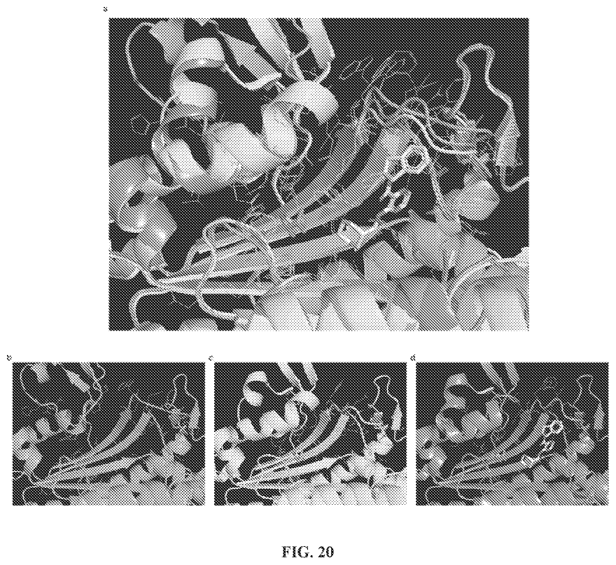

Biochemical profiling of these compounds, including against PfcProRS, revealed a difference in potency between the two inhibitor pairs, identifying compounds 30/32 as the most potent pair with equivalent potency in the absence and presence of proline (FIG. 17b-c). Unexpectedly, 32 (K_{D}=6.67 nM) has substantially higher affinity than the corresponding deprotected target compound MAT345 (K_{D}=39.8 nM). The lesser biochemical activity of proline-hybrids 29/31 was recapitulated by the lack of activity against ABS P. falciparum, whereas compound 32, which was of comparable potency to NCP26, and MAT345 inhibited parasite growth with EC_{50,Dd2-2D4}=63.2 nM and 249 nM, respectively (FIG. 17d). Like compound 7, the activity in halofuginone-induced and HFGR-I parasites was 20-40-fold decreased. Notably, only co-crystal structures for compounds MAT334 and MAT345 with PfcProRS were obtained in the presence, but not in the absence, of proline (PDB: 7QC2 and 7QB7, respectively, and FIG. 18). Analysis of both co-crystal structures revealed that proline occupies the amino acid binding pocket, while the prolyl-substituent of MAT334/345 is pointing outside the active site and does not form discernible electrostatic interactions with surrounding residues, which suggests the prolyl moiety is highly flexible in this binding mode (FIG. 17b). While unexpected, these results are not inconsistent with a lack of proline-uncompetitive binding as determined in biochemical assay, and a proline-competitive mode of inhibition as determined by cellular inhibitor profiling (FIG. 17c-e and FIG. 19) and do not demonstrate that the prolyl-substituent is unable to bind the amino acid pocket in the absence of proline, but rather show that crystallization efforts were unsuccessful in the absence of proline. Interestingly, the comparison of the crystal structures of apo (PDB: 4K86) and proline-bound (PDB: 7OSY) HsProRS shows a major reorientation of amino acid residues in the ATP-pocket upon proline binding, which closely resembles the proline and T-3767758 co-crystal structure (PDB: 5VAD), as well as the residues lining the active site rim, which interact with the prolyl-substituent of MAT334/345 (FIG. 20). Together, these observations provide a rationale for the proline-uncompetitive binding of the simple pyrazinamide-based ligands (e.g. NCP26) and the proline-competitive binding of the modified analogs (e.g., MAT334 and MAT345).

Analysis of the co-crystal structure of halofuginone and ATP with ProRS illustrates the molecular basis of halofuginone's ATP-uncompetitive binding mode. The ketone and hydroxyl-group of halofuginone form two well-defined hydrogen bonds with the α-phosphate of ATP, furnishing the intrinsic inhibitory complex in these ProRS ligand structures. The importance of ATP for halofuginone binding to PfcProRS and HsProRS is confirmed by biochemical data that show three orders of magnitude lower affinity in the absence of ATP (see FIG. 9).

### Discussion

This example provides generalizable high-throughput assay platform to support, e.g., the comprehensive biochemical profiling of ProRS inhibitors against the parasite and human paralogs. TR-FRET-based ligand displacement assay strategy resolves the limitations of current platforms that have stymied aaRS-targeted drug development and offers exceptional throughput, robustness, sensitivity, and flexibility. The methodology is based on a simple mix-and-read assay design that enables kinetic measurements and detailed interrogation of inhibition modes, while reducing the required amount of protein by several orders of magnitude. These characteristics not only improve economic aspects, but, more importantly, allow for the quantitative profiling of high-affinity ligands, which for the first time established accurate equilibrium binding constants for ProSA. The presented CoraFluor-ProRS technology greatly accelerates the drug discovery process beyond malaria and is equally applicable to other parasitic diseases where the corresponding ProRS homolog is a validated drug target, including toxoplasmosis, leishmaniasis, cryptosporidiosis, and coccidiosis. Moreover, host aaRSs have been recognized for their many roles in human health and disease, and HsProRS is an attractive target for the development of new drug classes for the treatment of autoimmune disorders, fibrosis, cancer, and more recently viral infections, including COVID-19, chikungunya, and dengue.

TR-FRET tracers MAT379 and MAT425 exhibited >15-fold and >50-fold reduced affinity for HsProRS relative to PfcProRS, respectively. This points to contributions of protein features adjacent to the active site. In fact, the region expected to be occupied by the FITC-functionalized linker represents one of the least conserved regions between PfcProRS and HsProRS (FIG. 8).

### Additional Discussion

Aminoacyl tRNA synthetase (aaRS) enzymes are desirable drug targets. aaRSs exist in all living cells and are indispensable enzymes in protein biosynthesis. In their canonical function they catalyze the transfer of amino acids to their cognate tRNAs. This process, generally referred to as “charging”, is highly specific and ensures the steady supply of aminoacyl-tRNAs that are used by the ribosome as the fundamental building blocks for protein synthesis. More recently, additional secondary, isoform-specific, functions of aaRSs have been recognized. aaRSs and associated pathways are attractive targets for chemotherapeutic intervention in a wide range of human diseases, such as cancer. autoimmune disorders, and infectious diseases, including bacterial, fungal, viral, parasitic infections. The general lack of robust, sensitive and straightforward biochemical and cellular assay platforms for aaRSs has broadly hampered the identification and rational development of inhibitors for this enzyme family. The identification and development of aaRS inhibitors has been greatly impeded by the lack of sensitive, robust, and straightforward biochemical assay platforms that allow for high-throughput screening and reliable ligand profiling. Although several biochemical aaRSs assay platforms have been reported, including for both PRS homologs, they suffer from several shortcomings. Because of the low turnover rate of aaRS and the lack of sensitive fluorogenic substrates non-radioactive aaRS assays generally require 0.1-0.5 μM enzyme ([E]). However, even if enzyme supply does not constitute a bottleneck, high enzyme concentration limits the accurate measurement of binding affinities to inhibitors with Kd-values >½[E], while more potent inhibitors will appear indistinguishable. Additionally, current assay platforms require multiple manipulation steps that are challenging to implement in HTS settings and arc prone to errors.

Disclosed herein is a ligand-displacement assay. This assay utilizes linker-modified active site-directed small molecule ligands that are labeled with a fluorophore that is suitable to function as TR-FRET acceptor. The TR-FRET donor molecule is installed on the aaRS of interest by different means, including direct covalent labeling, through an antibody directed at the aaRS or an epitope tag (e.g., His6-tag), or by expressing the aaRS of interest as a fusion protein with a self-labeling protein tag (e.g., HaloTag).

While ligand displacement assay platforms have been developed for other protein targets, no such assay has been reported for aaRSs. The reason for this is that no small molecule ligands that would allow for fluorophore labeling while retaining sufficiently high affinity for the target aaRS isoform have been reported. Hence, provided herein is a series of fluorophore labeled ligands (such as MAT379 and MAT425) based on a ProRS inhibitors that retain the ability to potently bind human and P. falciparum prolyl-tRNA-synthetase (PRS). These ligands are suitable as tracers for TR-FRET based ProRS assays. Advantageously, these assays reduce the procedure to a single step and require 100-1000-fold less enzyme, while simultaneously providing increased robustness, flexibility and sensitivity. The assay developed in this example is a generalizable approach that is applicable to other aaRS isoforms besides ProRS. Analysis of existing co-crystal structures of various aaRS isoforms with their substrates or small molecule inhibitors suggests that the ligands are generally bound deeply buried (comparable to ProRS), generally limiting the options for the attachment of a linker that would enable the installment of a fluorophore as TR-FRET acceptor or donor The common exception suggested by this analysis appears to be the position of triphosphate tail of ATP that is pointing outside the active site of aaRS and provides an exit vector that can be employed for tracer development. Using ProSNA, a linker-containing compound was prepared that exhibits nanomolar affinity for human and Plasmodium PRS. Similarly, a linker-modified ligand was prepared that is a formal hybrid of sulfamoyl amino adenosine and halofuginone. This is discussed in Example 2.

### General Methods for Assay Development (e.g., for Examples 1 and 2)

### Preliminary Docking Studies:

While the inhibitor classes were all rationally designed, preliminary docking studies were done to guide in the prioritization of which analogs to synthesize first. These studies were conducted in Spark™ v10.5.0. Forge™ v10.5.0, and Flare™ v4.0.2 (Cresset Biomolecular Discovery Ltd) per the manufacturer's instructions in their respective user guides. Ligands were docked against the ProRS structures reported here (PDB 6T7K, 7QB7, 7QC1, and 7QC2) and previously (for HsProRS, PDB: 5VAD, 4HVC, 4K86, 4K87, 4K88, and 5V58; for PfcProRS. PDB 4Q15, 4NCX, 4YDQ, 40LF, 5IFU, and 4WI1). Protein preparation was accomplished using default settings and the pharmacophore constraints were automatically generated and used without modification. Conformation hunts were done with “very accurate but slow” setting modified to allow rotation about acyclic secondary amide bonds. Alignments were performed using both “normal” (unbiased) and “substructure” (guided by ligands from crystal structures) settings. No model building was used to guide chemical synthesis.

### Protein Constructs, Expression, and Purification:

Genes encoding for HsProRS (residues 996-1512), UniProt accession ID P07814) and PfcProRS (residues 249-746, PF3D7_1213800) were codon optimized for expression in E. coli and subcloned (GenScript Biotech Corporation, Piscataway. New Jersey) into a pFN29A His6HaloTag T7 Flexi Vector (Promega), which contains an N-terminal His6-Tag-HaloTag (henceforth HT) followed by a linker sequence containing a TEV-cleavage sit

(5′-GAGCCAACCACTGAGGATCTGTACTTTCAGAGCGATAACGCGATCG
CC-3′).

The HT-PfcProRS and HT-HsProRS plasmids were independently transformed into SoluBL-21T™ E. coli (Genlantis Inc. #C700200) and single colonies were picked from lysogeny broth (LB)-agar-ampicillin plate. SoluBL-21™ E. coli expressing either HT-PfcProRS or HT-HsProRS were cultured in lysogeny broth supplemented with 100 μg/ml ampicillin at 37° C. until OD₆₀₀˜0.17, cooled to 15° C., induced with 0.1 mg/mL IPTG (isopropyl β-D-thiogalactopyranoside), and cultured overnight at 15° C. Cell pellets were collected via centrifugation for 20 min at 2,800×g, flash frozen with liquid nitrogen, and stored at −80° C. until lysis performed. Bacterial cell pellets were quickly thawed in room-temperature water and independently lysed on ice in B-PER Bacterial Protein Expression Reagent (Thermo Scientific #78243). pH 7.0 supplemented with 10 mM imidazole, 500 mM NaCl, 10 mM MgSO₄, 1 mM AEBSF, 1 mM dithiothreitol, 10% glycerol. 2 mM ATP, 25 U/mL Benzonase nuclease (Sigma Aldrich #E1014), and 9,000 U/mL Ready-Lyse lysozyme (VWR International #76081-780) until visually homogeneous and then for an additional 5 min. During lysis, samples were vortexed, sonicated, and pipetted up and down using a serological pipette. Cell lysates were clarified by centrifugation (2,800×g for 20 min followed by 21,000×g for 10 min).

Clarified lysate was purified on HisTrap HP column (VWR #89501-388). All steps were performed at I mL/min and 4° C. The column was pre-equilibrated with 10 mL water and 5 mL 10 mM imidazole in wash buffer (25 mM HEPES, pH 7.0, 500 mM NaCl, 1 mM DTT, and 10% glycerol). After clarified lysate was loaded, successively eluted with 5 mL of 20 mM imidazole in wash buffer, 5 mL of 40 mM imidazole in wash buffer. 5 mL of 300 mM imidazole in wash buffer, and then 5 mL of 500 mM imidazole in wash buffer.

Protein purity was assessed by sodium dodecyl sulfate polyacrylamide gel electrophoresis (SDS-PAGE) with 1.0-mm NuPAGE 4-12% Bis-Tris protein gels in NuPAGE MOPS running buffer at 120V. The HaloTag of HT-ProRS was labeled prior to sample preparation with 100 μM TAMRA-Halo (55) for 15 min at room temperature. Gels were analyzed using an Amersham Typhoon FLA 9500 fluorescence gel scanner (Cytiva Life Sciences; version 1.0.0.7; Cy3 excitation/emission) followed by Coomassie staining with SimplyBlue™ SafeStain (ThermoFisher #LC6060).

Protein concentration was measured by NanoDrop 1000 (ThermoFisher Scientific, version 3.8.1) per the manufacturer's instructions (HT-PfcProRS: molecular mass=86.1 kDa and extinction coefficient=155,000 M⁻¹·cm⁻¹; HT-HsProRS: molecular mass=94.7 kDa and extinction coefficient=150,000 M⁻¹·cm⁻¹).

Desired fractions based up were buffer exchanged into 25 mM HEPES, pH 7.0, 100 mM NaCl, 1 mM dithiothreitol, and 5% glycerol using PD-10 columns (Cytiva #17-0851-01) per the manufacturer's instructions.

For long-term storage, ProRS protein stocks were aliquoted following addition of glycerol to 20%, flash frozen in liquid nitrogen, and stored at −80° C.

For crystallization studies, HT-PfcProRS was expressed in a phage-resistant derivative of Escherichia coli strain BL21(DE3) carrying the pRARE2 plasmid for rare codon expression. Cells were grown at 37° C. in Terrific Broth supplemented with 100 μg/mL ampicillin until the culture reached an OD₆₀₀ of 2.0. The temperature was then decreased to 18° C. and protein expression induced with 0.5 mM IPTG (isopropyl P-D-thiogalactopyranoside) overnight. Cells were collected by centrifugation and resuspended in 50 mM HEPES, pH 7.5, 500 mM NaCl, 10 mM Imidazole, 5% glycerol, 0.5 mM TCEP, a protease inhibitor cocktail (Sigma), lysozyme, and benzonase, and lysed by sonication. The cell lysate was clarified by centrifugation and the proteins purified by nickel-affinity chromatography (Cytiva) using a stepwise gradient of imidazole. The His6-Tag-HaloTag fusion (HT) was removed by incubating with TEV protease at 4° C. overnight and this was followed by size exclusion chromatography (Superdex 200, Cytiva) in 20 mM MES, pH 6.0, 250 mM NaCl, 5% glycerol, and 0.5 mM TCEP. The TEV protease, cleaved byproducts containing histidine tag, and unreacted HT-PfcProRS were removed by nickel-affinity chromatography and concentrated using an Amicon centrifugal filtration unit. The mass of purified protein was verified by electrospray ionization time of flight mass spectrometry (ESI-TOF-TOF: Agilent LC/MSD).

### CoraFluor-1-Halo Labeling of HT Fusion Proteins:

A freshly thawed solution of HT-ProRS in storage buffer (25 mM HEPES, pH 7.0, 100 mM NaCl, 1 mM dithiothreitol, and 20% glycerol) was incubated with 5 molar equivalents CoraFluor-1-Halo overnight at 4° C. Unreacted CoraFluor-1-Halo was removed by buffer exchanging with 7 k MWCO Zeba spin desalting columns (ThermoFisher Scientific #89883) into 25 mM HEPES, pH 7.0, 100 mM NaCl, 1 mM dithiothreitol, and 5% glycerol according to manufacturer's protocol.

Protein concentration following CoraFluor-1-labeling was semi-quantitatively measured by NanoDrop and corrected for absorbance by CoraFluor-1-Halo by the following equation where the correction factor is the ratio of A280/A340=0.1571 for CoraFluor-1-Halo: Protein Concentration in μM=((A280−(A340*correction factor))/E)/path length)*1,000,000.

The concentration of active HT-ProRS concentration following CoraFluor-1-Halo-labeling was quantitively measured by active-site titration of Cora-Fluor-labeled HT-ProRS (200 nM by nanodrop) with ProSA (25) in the presence of 250 nM MA379 (2.5×K_{D} for HT-PfcProRS and 0.15×K_{D} for HT-HsProRS) to determine the IC₅₀, calculating the apparent K_{D} using the Cheng Prusoff equation, and doubling the apparent K_{D} value (Equation 1). This allowed for accurate K_{D} determination for inhibitors suffering from ligand depletion (Equation 2) in the TR-FRET assay.

$$[ active HT - Pro RS ] = 2 \times \frac{{IC}_{50}}{1 + \frac{[ MAT 379 ]}{{K}_{D , MAT 379}}} \qquad ( Equation 1 )$$

$${IC}_{50 , Ligand Depletion} < 10 \times [ active HT - Pro RS ] \qquad ( Equation 2 )$$

For long-term storage at −80° C., glycerol was added to 20% and samples were flash-frozen with liquid nitrogen.

### Time-Resolved Föster Resonance Energy Transfer (TR-FRET) Measurements:

Experiments were performed in white, 384-well microtiter plates (Corning 3572 or Greiner 781207). TR-FRET measurements were acquired on a Tecan SPARK plate reader with SPARKCONTROL software version V2.1 (Tecan Group Ltd.), with the following settings: 340/50 nm excitation, 490/10 nm (Tb) and 520/10 nm (FITC) emission, 100 μs delay, 400 μs integration. The 490/10 and 520/10 emission channels were acquired with a dichroic 510 mirror, using independently optimized detector gain settings. The TR-FRET ratio was taken as the 520/490 nm intensity ratio on a per-well basis.

### Determination of Equilibrium Dissociation Constant (K_{D}) of Tracers MAT379 and MAT425 Toward ProRS Paralogs by TR-FRET:

A stock solution containing CoraFluor-1-Halo-labeled HT-PfcProRS or CoraFluor-1-Halo-labeled HT-HsProRS at the concentration specified for each experiment was prepared in assay buffer (50 mM Tris, pH 7.5, 20 mM KCl, 10 mM MgCl₂, 0.05% Tween-20, 1 mM dithiothreitol, and 0.5 mg/mL BSA). A Multidrop Combi Reagent Dispenser (ThermoFisher Scientific) was used to dispense 40 μL protein solution into wells of a white, 384-well plate (Corning 3572). Tracer MAT379 (24) or MAT425 was dispensed in dose-response in sextuplicate using a D300 digital dispenser (Hewlett Packard). Half the wells received 10 μM ProSA for background correction. Plates were mixed on an Ika MTS 2/4 Digital Microtiter Shaker at 750 rpm for 2 min, centrifuged at 1,000×g at 25° C. for 1 min. and allowed to equilibrate at room temperature for 2 h before TR-FRET measurements were taken. Specific signal was determined by subtracting raw values from wells containing 10 μM ProSA (25). PRISM 9 (GraphPad) was used to perform non-linear regression analysis (one site—specific binding), plot dose-response curves, and calculate K_{D} values.

Determination of Pro and ATP equilibrium dissociation constants (K_{D} values) for individual, recombinant ProRS paralogs via TR-FRET-based ligand displacement assay: A stock solution containing CoraFluor-1-Halo-labeled HT-PfcProRS (5 nM) or CoraFluor-1-Halo-labeled HT-HsProRS (1.5 nM) and tracer MAT379 (50 nM for HT-PfcProRS and 1,000 nM for HT-HsProRS) was prepared in assay buffer (50 mM Tris, pH 7.5, 20 mM KCl, 10 mM MgCl₂, 0.05% Tween-20, 1 mM dithiothreitol, and 0.5 mg/mL BSA). Prepared serial dilutions (octuplicate, 18-point, 1:2) of proline (cₘₐₓ=50 mM) and ATP (cₘₐₓ=100 mM) and dispensed 20 μL per well into flat, white, 384-well plates (Corning 3572). A Multidrop Combi Reagent Dispenser (ThermoFisher Scientific) was used to dispense 20 μL of 2× protein solution into each well. Using a D300 digital dispenser (Hewlett Packard), dispensed 10 μM ProSA into half the wells of each dose of substrate for background correction. Plates were mixed on an Ika MTS 2/4 Digital Microtiter Shaker at 750 rpm for 2 min, centrifuged at 1,000×g at 25° C. for I min, and allowed to equilibrate for 2 h at room temperature before TR-FRET measurements were taken.

PRISM 9 (GraphPad) was used to perform non-linear regression analysis (log(inhibitor) vs. response—Variable slope (four parameters)), plot dose-response curves, and calculate iC₅₀ values. The Cheng Prusoff equation was used to convert IC₅₀ to K_{D} values (Equation 3).

$${K}_{D} = \frac{{IC}_{50}}{1 + \frac{[ MAT 379 ]}{{K}_{D , MAT 379}}} \qquad ( Equation 3 )$$

### Determination of ProRS Affinity and Substrate Binding Mode by Time-Resolved Forster Resonance Energy Transfer Assay:

TR-FRET was used assay to determine the affinity (equilibrium dissociation constants, K_{D} values) and binding mode (competitive or noncompetitive versus uncompetitive) with respect to substrates (proline and ATP) of our test compounds. Note that the substrate concentrations used here (0 μM proline+0 μM ATP; 100 μM proline+0 μM ATP; or 0 μM proline+500 μM ATP) were chosen to identify uncompetitive inhibitors without substantial competition to the tracer MAT379 which is competitive with both ATP and proline. However, the ATP and proline concentrations used are not substantially above the substrates K_{D} values to facilitate differentiation of substrate-noncompetitive and substrate-competitive inhibitors because these substrate concentrations would compete with our tracer and because it was explicitly sought to develop proline-uncompetitive ProRS inhibitors to circumvent or overcome halofuginone-resistance mechanisms.

A stock solution containing CoraFluor-1-Halo-labeled HT-PfcProRS or CoraFluor-1-Halo-labeled HT-HsProRS at the concentration specified for each experiment and 250 nM tracer MAT379 (24) was prepared in assay buffer (50 mM Tris, pH 7.5, 20 mM KCl, 10 mM MgCl₂, 0.05% Tween-20, 1 mM dithiothreitol, and 0.5 mg/mL BSA). and, where indicated, supplemented with 100 μM proline or 500 μM ATP. A Multidrop Combi Reagent Dispenser (ThermoFisher Scientific) was used to dispense protein solution (30 or 40 μL) into each well of a flat, white, 384-well plate (Corning 3572 or Greiner 781207). Test compounds were dispensed in duplicate, triplicate, or sextuplicate dose-response format using a D300 digital dispenser (Hewlett Packard). Each plate included blank wells (no-inhibitor negative control for assay ceiling) and wells receiving 10 μM ProSA (25, positive control for assay floor) for Z-factor determination and a dose-response of NCP26 (3) as a standard. Plates were mixed on an Ika MTS 2/4 Digital Microtiter Shaker at 750 rpm for 2 min, centrifuged at 1,000×g at 25° C. for 1 min, and allowed to equilibrate for 2 h at room temperature.

Z-factors were calculated in Excel using 10 μM ProSA wells and negative control wells. GraphPad PRISM was used to perform non-linear regression (log(inhibitor) vs. response—Variable slope (four parameters)), plot dose-response curves, and calculate IC₅₀ values. The ligand-depletion corrected Cheng Prusoff equation was used to convert IC₅₀ to K_{D} values (Equation 4). K_{D_appMAT379} is defined as MAT379's K_{D} corrected for the concentration of proline or ATP, if any, using the Cheng Prusoff equation. Note that for this equation, the [active HT-ProRS]was the active ProRS concentration determined by titration with ProSA (see above).

$${K}_{D} = \frac{{IC}_{50} - 0.5 \times [ active HT - Pro RS ]}{1 + \frac{[ MAT 379 ]}{{K}_{D \_ app , MAT 379}}} \qquad ( Equation 4 )$$

The inhibition mode for each test compound with respect to ATP or proline was determined by comparing the K_{D} values measured in the presence and absence of each substrate.

As a reminder, Equation 4 is only valid when the active HT-ProRS concentration is >˜2×K_{D}. All values reported in the text or tables are not from ProRS-titrating conditions, but in some plots, compounds are titrating and these are clearly indicated in the figure legend (FIG. 9d-f and FIG. 14b,c,e). The anti-His6 antibody format (see below) was utilized to enable accurate determination of ProSA's affinity (K_{D} value) and this data is shown for ProSA in FIG. 9g, Table 1.

### Time Resolved Forster Resonance Energy Transfer (TR-FRET) Inhibition Mode Determination—Anti-His6 Antibody Format:

This assay was generally conducted in the same manner as the CoraFluor-1-Halo format with minor differences. All assays were conducted in sextuplicate dose-response with CoraFluor-1-Halo-labeled HT-ProRS whose concentration was determined by titration with ProSA. Each well was supplemented with 1 nM CoraFluor-1-Pfp-labeled anti-His6 antibody before the 2 h incubation.

The commercially available anti-His6 antibody (Abcam ab18184) was labeled as described previously. The following extinction coefficients were used to calculate antibody concentration and degree-of-labeling (DOL): Antibody E₂₈₀=210,000 M⁻¹cm⁻¹, CoraFluor-1-Pfp E₃₄₀=22,000 M⁻¹cm⁻¹. Antibody conjugates were diluted with 50% glycerol, flash-frozen in liquid nitrogen, and stored at −80° C.

### TR-FRET Binding Kinetics:

Dissociation rates (k_{off}) of the TR-FRET tracer MAT379 (24) and HT-ProRS homologs were measured by rapid dilution (n=23) in white, 384-well plates. An equilibrated solution of 5 μL 100 nM CoraFluor-1-Halo-labeled HT-ProRS, ˜EC₈₀ MAT379 (560 nM MAT379 for HT-PfcProRS and 7 μM MAT379 for HT-HsProRS), and either 10 M ProSA (˜20,000×K_{D}) or DMSO vehicle in assay buffer (50 mM Tris, pH 7.5, 20 mM KCl, 10 mM MgCl₂, 0.05% Tween-20, 1 mM dithiothreitol, and 0.5 mg/mL BSA) was diluted 10-fold into 45 μL assay buffer using a multichannel pipette, and briefly mixed by pipetting up and down three times. TR-FRET measurements were acquired in kinetic mode (1 read every ˜45 s) for at least 10 min. Excel was used to subtract the background signal (10 μM ProSA wells) from the DMSO vehicle wells. PRISM 9 (GraphPad) was used to perform non-linear regression (Dissociation—One Phase exponential decay), plot 520/490 nm TR-FRET ratio vs. time, and calculate k_{off} values.

The association rates (k_{on,obs}) were measured using a similar method (described below), but they were too fast to measure (fully equilibrated by first time point) calculated the association rates (k_{on,calc}) using the measured dissociation rates (koff) and measured equilibrium dissociation constants (K_{D}) in Equation 5.

$${k}_{on , calc} = {k}_{off} / {K}_{D} \qquad ( Equation 5 )$$

The attempts to measure association rates (k_{on,obs}) of the TR-FRET tracer MAT379 and HT-ProRS homologs were similarly performed in dilution format (n=23) in white, 384-well plates. An equilibrated solution of 5 μL 100 nM CoraFluor-1-Halo-labeled HT-ProRS and either 10 μM ProSA or DMSO vehicle in assay buffer was diluted 10-fold into 45 μL MAT379 (500 nM MAT379 for HT-PfcProRS and 5 μM MAT379 for HT-HsProRS) in assay buffer using a multichannel pipette, and briefly mixed by pipetting up and down three times. TR-FRET measurements were acquired in kinetic mode (1 read every ˜45 s) for at least 10 min. Excel was used to subtract the background signal (10 μM ProSA wells) from the DMSO vehicle wells. GraphPad PRISM was used to perform non-linear regression (Association kinetics—One Cone. of hot), plot 520/490 nm TR-FRET ratio vs. time, and calculate k_{on,obs} values. However, as noted above, this failed to provide meaningful k_{on,obs} values because the samples were fully equilibrated by the first scan, and thus too fast to measure.

### Crystallization, Data Collection and Structure Determination:

PfcProRS was co-crystallized with NCP26 (3), MAT334 (29), and MAT345 (30) at 20° C. using the sitting drop vapor diffusion method.

For crystals of PfcProRS in complex with NCP26 and proline (PDB: 6T7K), 2 mM NCP26 was added to 39 mg/mL PfcProRS together with 5 mM L-Proline, and crystals were obtained in a drop containing 75 nL of protein-compound mixture and 75 nL precipitant composed of 0.1 M HEPES, pH 7.5, and 20% PEG 10000.

For crystals of PfcProRS in complex with MAT334 and proline (PDB: 7QC2), MAT334 was added to PfcProRS (3 mg/mL) at a concentration of 0.5 mM, and the protein-compound mixture incubated 30 min on ice before it was concentrated to 28.5 mg/mL. Crystals of PfcProRS in complex with MAT334 and proline were obtained in a drop containing 75 nL of protein-compound mixture and 75 nL precipitant composed of 0.2 M L-Proline, 10% PEG3350, and 0.1 M HEPES, pH 7.5.

Crystals of PfcProRS in complex with MAT345 and proline (PDB: 7QB7) were obtained in a drop containing 75 nL of a protein-compound mixture with 1 mM of MAT345, 5 mM L-proline, and 22 mg/mL PfcProRS, and 75 nL precipitant compost of 25% PEG3350 and 0.1 M BIS-TRIS, pH 6.5.

The crystals were cryo-protected in precipitant solution supplemented with 25-30% ethylene glycol and then flash cooled in liquid nitrogen. Data was collected on beamlines 103 and 104 at the Diamond Light Source UK, and the dataset processed, scaled, and merged at the Diamond Light Source using Xia2.⁵³ Electron density maps were obtained by molecular replacement using PHASER with previously determined structures of PfcProRS as a search model.

The complex structure of PfcProRS with NCP26 (PDB 6T7K) was solved to 1.79 Å resolution using PDB 4Q15 as a search model. The complex structure of PfcProRS with MAT334 was solved to 2.28 Å resolution (PDB 7QC2), MAT345 to 1.92 Å (PDB 7QB7), using PDB 6T7K as search model. The structures were refined in an iterative process using PHENIX with electron density map inspections and model improvement in WinCOOT and terminated when there were no substantial changes in the R_{work} and R_{free} values and inspection of the electron density map suggested that no further corrections or additions were justified. Structural analysis and figures were performed with PyMOL.

Crystallographic data and refinement statistics are available in FIG. 18.
P. falciparum Cell Lines and Culture Conditions:

Parasites were maintained under standard culture conditions as described previously. The P. falciparum Dd2-2D4 clone was derived from Malaria Research and Reagent Resource Repository line MRA-156 (BEI Resources). The P. falciparum HFG-induced (elevated proline homeostasis) and HFGR1 (elevated proline homeostasis and PfcProRS^{L482H}) were previously reported previously.

P. falciparum Asexual Blood Stage Growth Assay:

This assay was performed as previously described. In short, P. falciparum erythrocytic-stage parasites at 1% parasitemia and 1% hematocrit in RPMI+0.5% Albumax were seeded at 40 μL/well in 384-well plates with test compounds in triplicate, dose-response format with 10 μM dihydro-artemisinin as a kill-control and blank (no compound) wells as a growth-control. DMSO concentration did not exceed 1% (v/v). After 72 h, growth was quantified by measuring fluorescence following SYBR Green staining. Data was analyzed in Excel and plotted in GraphPad PRISM.

P. falciparum Asexual Blood Stage Short-Term Resistance Susceptibility Assay:

Using the robust procedure previously used to generate HFG-induced parasites (HFG-tolerant with elevated proline homeostasis), unsuccessful attempts were made to generate NCP26-tolerant/resistant parasites. In short, three independent flasks of P. falciparum Dd2-2D4 parasites were treated with 4×EC₅₀ NCP26 until no parasites were detected by Giemsa staining microscopy. Following recrudescence, sensitivity to NCP26 and halofuginone was assayed using the ABS growth assay.

### NCP26 Resistance Selection:

Three independent selections for NCP26-resistant mutants of P. falciparum Dd2-2D4 parasites were conducted in vitro as previously reported. In short, parasites were treated with 4×EC₅₀ NCP26 until no parasites were detected by giemsa staining microscopy. Following recrudescence, the asexual blood stage growth assay was used to determine sensitivity to NCP26 and control compounds including ProRS inhibitors halofuginone (1), halofuginol (26), and ProSA (25); threonyl-tRNA synthetase (ThrRS) inhibitor borrelidin; and dihydroartemisinin (DHA). This cycle was repeated for ˜50 generations (˜100 days), corresponding to 5-6 cycles of drug pressure. Selections were initially made with ˜3×10⁸ parasites per flask (i.e. per independent selection), but did not observe any resistance after 2 cycles of drug pressure (38 days; ˜19 generations) so selection cultures were expanded to ˜1×10⁹ parasites per flask and maintained this for the remainder of the selection.

### Subcloning:

Clonal parasites were isolated from each selection flask by limiting dilution of ring stage parasites in 96-well plates to an average of 0.8 and 0.2 parasites per well. Following recrudescence, these clonal parasites were assayed in the asexual blood stage blood stage viability assay to ensure no phenotypic differences from the corresponding bulk population (all isolated clones had EC₅₀ values for all inhibitors tested within 2-fold of corresponding bulk population).

### Library Preparation and Whole Genome Sequencing:

Infected RBCs were washed with 0.05% saponin and genomic DNA was isolated from the parasites using a DNeasy Blood and Tissue Kit (Qiagen) according to the standard protocols. Sequencing libraries were prepared with the Nextera XT kit (Cat. No FC-131-1024, Illumina) via the standard dual index protocol and sequenced on the Illumina NovaSeq 6000 S4 flow cell to generate paired-end reads 100 bp in length. Sequence data is available under BioProject Accession number: PRJNA811614 in the NCBI Sequence Read Archive. Reads were aligned to the P. falciparum 3D7 reference genome (PlasmoDB v13.0) using the previously described pipeline. A total of 8 samples were sequenced to an average whole genome coverage of 157×, with an average of 89% of reads mapping to the reference genome. Following alignment, SNVs and INDELs were called using GATK HaplotypeCaller and filtered according to GATK's best practice recommendations. Variants were annotated using a custom SnpEff database and further filtered by comparing those from resistant clones to the parent clone, such that only a mutation present in the resistant clone but not the sensitive parent clone would be retained. CNVs were identified by differential Log 2 copy ratio as described in the GATK 4 workflow. Briefly, read counts were collected across genic intervals for each sample. Copy ratios were calculated after denoising read counts against a strain-matched Panel of Normals composed of non-drug-selected Dd2 parasite samples.

### PCR Amplification and Sanger Sequencing:

Genomic DNA was isolated as described above (see Library preparation and whole genome sequencing). Sections of the cPRS gene were amplified by polymerase chain reaction (PCR) to validate the mutations observed by whole genome sequencing. Primers (single stranded DNA oligomers) were ordered from Integrated DNA Technologies Inc (see PCR Primers Table below for sequences). Immediately prior to PCR reaction, combined 2.5 μL 5 μM forward primer+5 μM reverse primer in 1×TE buffer (10 mM Tris, pH 7.5, 1 mM EDTA) or 2.5 μL 1×TE buffer (no primer control) with 10 μL 1 ng/μL isolated gDNA, mixed by briefly vortexing, and pulse spun. Added 12.5 μL 2×GoTaq G2 Colorless Mastermix, mixed by briefly vortexing, and pulse spun. PCR reactions were performed on thermocycler (Eppendorf AG 22331 Hamburg No. 5341) with the following method: 95° C. for 2 min; 30 cycles of 95° C. for 1 min, 55° C. for 1 min, and 73° C. for 1 min; and then 73° C. for 5 min.

PCR reactions were analyzed by 1% agarose gel electrophoresis and fluorescently imaged following ethidium bromide staining to ensure PCR reactions produced one product. DNA was purified from PCR reactions using Zymo DNA Clean and Concentrator-5 Kit (Zymo Research #D4005). Purified DNA was submitted to Genewiz Inc for Sanger sequencing and results were aligned to the predicted and sequenced results from the Dd2-2D4 parent line using Benchling.

Note that for the PfcProRS^{F405L} mutation (S3), direct analysis of the raw fluorescence intensity data was required as sanger sequencing samples were called as wildtype by Genewiz despite having ˜50% as much signal for the PfcProRS^{F405L} mutant allele. This is consistent with whole genome sequencing data.

PCR Primers:

| Pair | Mutations | | |
|---|---|---|---|
| Name | Recognized | Forward Primer Sequence | Reverse Primer Sequence |
| F2-R2 | N/A | 5′-ACATGGCTTAGCTGCTACTAT | 5′-TCCTTAGATTTGTTATCTTCT |
| | | T-3′ | ACTTCATTAT-3′ |
| F3-R3 | c.1535C > G | 5′-TTTAATCACGCTCCTGAATAT | 5′-AATTGTTTCACTTGTTGGTCT |
| | (PfcProRS_{T512S}) | GT-3′ | TATAG-3′ |
| F4-R4 | c1213T > C | 5′-CCGCTGCATATTATATATGGG | 5′-AGAAACATTTGGTGGTAGAAC |
| | (PfcProRS_{F405L}), | AATG-3′ | TAATC-3′ |
| | and | | |
| | c.1535 C > G | | |
| | (PfcProRS_{T512S}) | | |
| F5-R5 | N/A | 5′-TGGTATGAAGAATATCTAGCT | 5′-CAACAAGCATTTGTTGTGTTT |
| | | GTACC-3′ | CTAA-3′ |
| F6-R6 | N/A | 5′-GAGCCATACAAGCAGCTAC | 5′-GGTGGTTGATCTAACGGGATA |
| | | A-3′ | C-3′ |
| F7-R7 | N/A | 5′-GTTTATGATGATAGAGCATCT | 5′-GAAAAATGGGGTACATATATA |
| | | TATTC-3′ | CAAATATAT-3′ |

P. berghei Liver Stage and HuH7 Host Hepatocyte Growth Assay:

HuH7 cells (Sigma) were cultured in DMEM+L-Glutamine (Gibco) supplemented with 10% (v/v) heat-inactivated FBS (Sigma) and 1% (v/v) antibiotic/antimycotic (Sigma). Hepatocyte cultures were maintained in a standard tissue culture incubator at 37° C. Anopheles mosquitoes infected with luciferase-expressing P. berghei ANKA sporozoites were obtained from the Sporocore at the University of Georgia. Liver stage P. berghei assays were completed as previously described. Briefly. 4,000 HuH7 cells were seeded into 384-well plates (Corning) one day prior to infection. Compounds (0-50 μM) were added in triplicate to wells before infection with 4,000 P. berghei sporozoites. At ˜44 bpi, HuH7 cell viability and P. berghei parasite load was assessed using CellTiter-Fluor (Promega) and Bright-Glo (Promega), respectively, using an Envision plate reader. Relative fluorescence and luminescence signal intensities were normalized to the negative control, 1% DMSO. EC₅₀ values were determined using GraphPad Prism through fitting data to a dose response curve. Reported EC₅₀ values are averages of three independent experiments.

### Example 2—Additional Assay Development

### General Methods

### Protein Constructs, Expression, and Purification:

HT-PfcProRS and HT-HsProRS were expressed and purified as described herein and elsewhere. The remaining constructs were provided by Dr. Vadim Baidin. All constructs except MtbPheRS were expressed in E. coli. The MtbPheRS was expressed in Mycobacterium. Following lysis and clarification by centrifugation, samples were successively purified by Ni-NTA affinity chromatography and size exclusion chromatography. Protein purity was analyzed by SDS-PAGE followed by Coomassie staining.

Labeling with CoraFluor-1-Halo and CoraFluor-1-Pfp: as previously described. TR-FRET Assay: Except the HsGluProRS lysate assays (described below), all TR-FRET assays were performed as described in Chapter 2, but with the indicated aaRS enzyme. These TR-FRET assay were performed in the format described in Chapter 2 methods: “Determination of ProRS Affinity and Substrate Binding Mode by Time-Resolved Förster Resonance Energy Transfer Assay” and “Time Resolved Förster Resonance Energy Transfer (TR-FRET) Inhibition Mode Determination-anti-His6 antibody format”. Please note that many of the assays described in this chapter have only one experimental replicate (not technical replicate).

P. falciparum Cell Lines and Culture Conditions, P. falciparum Asexual Blood Stage Growth Assay, PheRS Activity Assay:

Performed as described previously.

HsGluProRS TR-FRET with in HEK293 Lysate:

Freshly prepared HEK293 cell lysates were serial diluted into flat-bottom, white 384-well plates (Corning 3572). A D300 digital dispenser (Hewlett Packard) was used to add constant concentration of either GluRS tracer MAT579 (250 nM) or ProRS tracer MAT574 (250 nM=3.5×K_{D}), and of detection mixture consisting of either “5 nM unlabeled ab31531 anti-EPRS polyclonal rabbit IgG antibody+10 nM CoraFluor-1-Pfp-labeled CTK0101 anti-Rabbit-IgG secondary nanobody”, “5 nM CoraFluor-1-Pfp-labeled Proteintech 67712-1-Ig anti-EPRS monoclonal mouse IgG2a antibody”, or “5 nM unlabeled CST45956 anti-EPRS polyclonal rabbit IgG antibody+10 nM CoraFluor-1-Pfp-labeled CTK0101 anti-Rabbit-IgG secondary nanobody”.

Abcam ab31531 was raised against an Abcam-proprietary, recombinant, full-length HsGluProRS protein. Proteintech 67712-1-Ig was raised against a peptide encoding HsGluProRS residues 1163-1512. Cell Signaling Technology CST45956 was raised against a peptide encoding residues surrounding HsGluProRS^{P978}. CTK0101 (ChromTek) anti-Rabbit-IgG secondary nanobody (single domain nanobody) was previously labeled with CoraFluor-1-Pfp and validated to bind Rabbit IgG antibodies with high affinity.

### Reagents and Chemical Synthesis:

All reagents were purchased from Chem-Impex International Inc., Combi-Blocks Inc., Oakwood Chemical, Sigma Aldrich, Fisher Scientific International Inc., VWR International, and BioSynth CarboSynth and were used without purification. Stock solutions of inhibitors were prepared at 10 mM in molecular biology grade DMSO (Sigma Aldrich). Preparation of TAMRA-Halo and the TR-FRET donor CoraFluor-1 reagents (CoraFluor-1-Halo and CoraFluor-1-Pfp) were reported previously. The synthesis of MAT379, MAT425, and ProSA are described herein.

### Results

Non-hydrolyzable aminoacyl-AMP analogs, particularly 5′-N-linked aminoacyl sulfamoyladenosine analogs, are suitable TR-FRET tracers for the generalization of TR-FRET assay platform to several other aaRS isoforms from diverse bacterial and eukaryotic species including humans, P. falciparum (malaria). M. tuberculosis, S aureus, E. coli, P. aeruginosa and A. thaliana. This includes cytoplasmic, mitochondrial, and chloroplast isoforms. The data presented herein can be generalized to all isoforms, which enables generation of aaRS inhibitors, useful for a wide range of disorders such as those described herein (e.g., cancer, fibrosis, autoimmune disorders, and diverse infections including those caused by bacteria, fungi, malaria and other parasites, and viruses).

As with developing the TR-FRET assay for ProRS, these assays required two things: labeling the target aaRS with the CoraFluor-1 TR-FRET donor (e.g. labeling HaloTag-aaRS with CoraFluor-1-Halo as done for ProRS in Example 1, or direct chemical labeling with CoraFluor-1-Pfp) and developing a fluorescently-labeled tracer that could be displaced by test compounds. Design of tracers is presented herein.

The pyrazinamide series (described in Example 1), including TR-FRET tracers MAT379 and MAT425, are ProRS specific by virtue of their indane moiety which binds a ProRS-unique auxiliary pocket. The only reported chemotype which can bind all aaRS enzymes are non-hydrolyzable aminoacyl-AMP analogs, including the aminoacyl sulfamoyladenosine (aaSA) analogs. A related series known as aminoacyl sulfamidyladenosine (aaSNA) analogs, where the aaSA acylsulfamate moiety is replaced by an acylsulfamide have been reported for all 20 aaRS isoforms as being active in a tissue culture viability assay (CellTSterGlo) against LNCaP prostate adenocarcinoma cells:

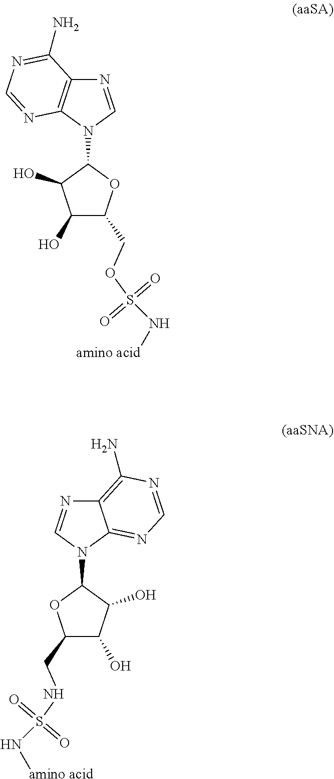

In this example, the amino acid is any one of 20 natural amino acids, including alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

Conveniently, these compounds, particularly the aaSA analogs, bind with extremely high affinity. ProSA has mid-picomolar K_{D} for both PfcProRS and HsProRS) which suggested sufficiently high affinity probes even if the introduction of the linker reduced their affinity. Further, the parent aaSA analogs could be used as control inhibitors when developing the assays.

### Prolyl-tRNA Synthetase

We initially focused on ProRS because (1) we could use our existing assay to screen potential tracer chemotypes without having to install a fluorescent label, and (2) our existing tracers provided a positive control and means to validate results. Detailed analysis of the co-crystal structures of ProSA bound to PfcProRS (unpublished data) and of the azetidine analog bound to HsProRS (PDB 5V58) indicated that the ribose 5′-oxygen and purine C8 were the two sites best suited for functionalization to create linker-modified analogs. Comparison with the structures published for other aaRS isoforms published on the PDB indicated that C8 was not always exposed, so the 5′-oxygen was selected for functionalization. This choice was later supported by results from our protein-ligand docking.

Since the chosen oxygen already had a valency of 2, it was formally replaced by a nitrogen, resulting in ProSNA

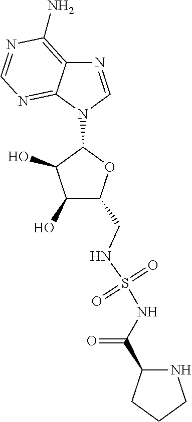 [SMILES: Nc1ncnc2c1ncn2[C@@H]1O[C@H](CNS(=O)(=O)NC(=O)[C@@H]2CCCN2)[C@@H](O)[C@H]1O]

which was readily accessed as shown in the examples. Notably, the protected precursors to ProSNA (MAT495 and MAT498) proved to be more chemically stable than those for ProSA due to the inability to form the N³,5′-cycloadenosine byproduct, improving yields and significantly reducing the effort to purify synthetic intermediates. Profiling in TR-FRET assay indicated that ProSNA had high affinity for both PfcProRS (K_{D}<1 nM) and HsProRS (K_{D}<1 nM). Also ProSNA is comparably active in wildtype asexual blood stage P. falciparum Dd2-2D4 parasites (EC₅₀=172 nM; c.f. ProSA EC₅₀=151 nM).

Linker to use was then investigated. A 5′-N-alkylated analog of ProSNA (MAT556) via the scheme shown in examples and determined the affinity for PfcProRS (K_{D}=195 nM) and HsProRS (K_{D}=33.7 nM) (see figures).

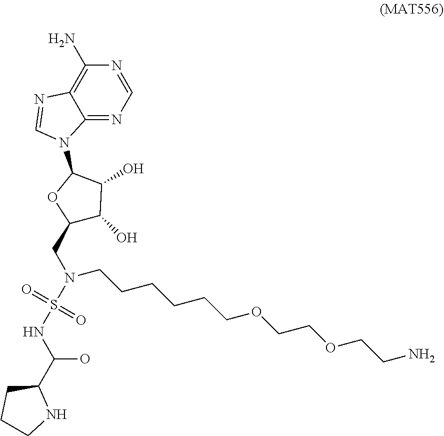 [SMILES: NCCOCCOCCCCCCN(C[C@H]1O[C@@H](n2cnc3c(N)ncnc32)[C@H](O)[C@@H]1O)S(=O)(=O)NC(O)[C@@H]1CCCN1]

Notably, while the affinity of MAT566 is substantially worse than that of ProSA and ProSNA, it is similar to that of MAT379 for PfcProRS and what others have suggested may be the ideal tracer affinity for primary screening (˜100 nM).

Encouraged by these results, MAT574 was prepared, a fluorescein-labeled analog of MAT556, as shown in the examples.

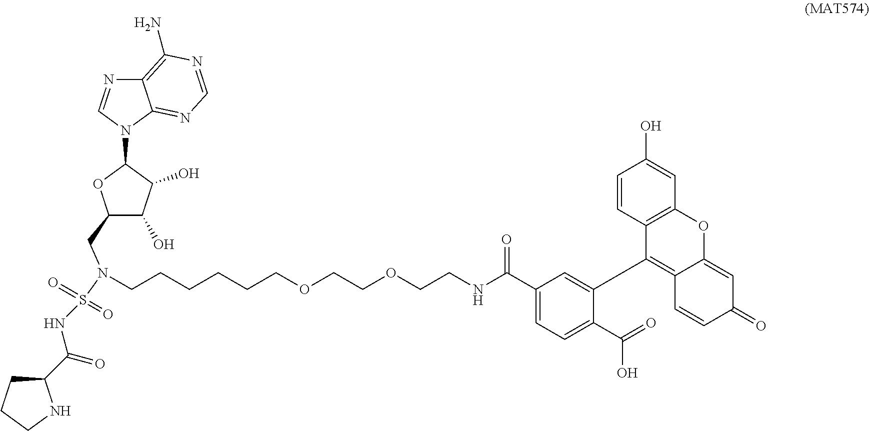 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H]2CCCN2)[C@@H](O)[C@H]1O]

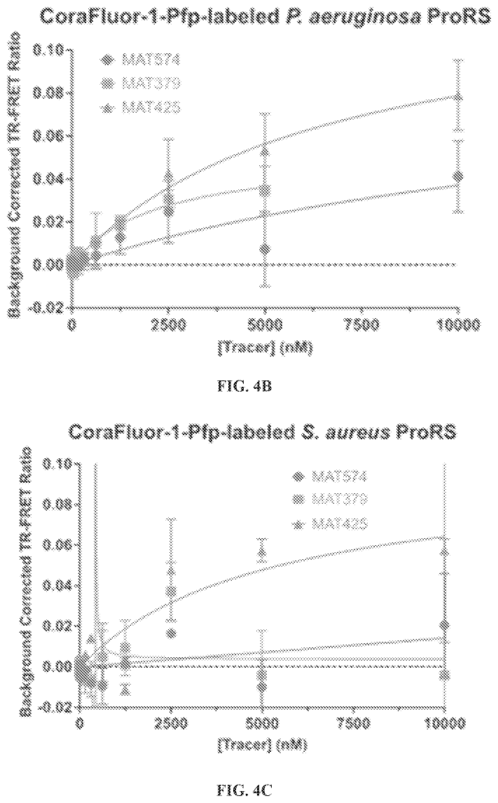

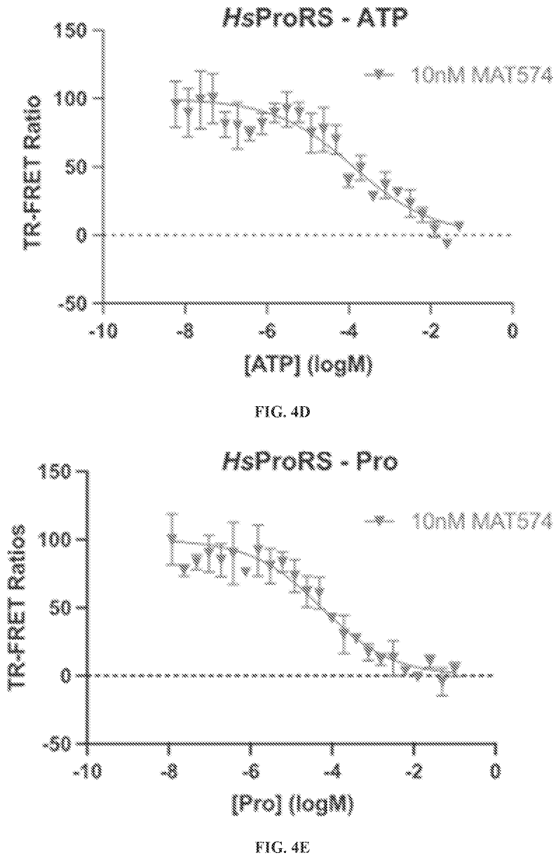

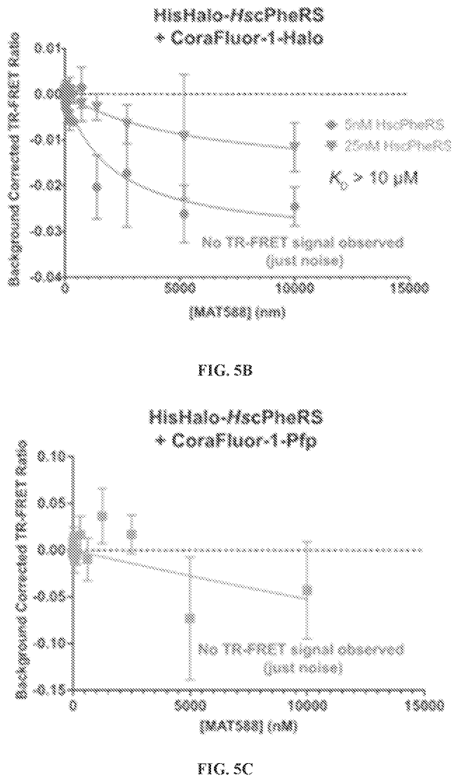

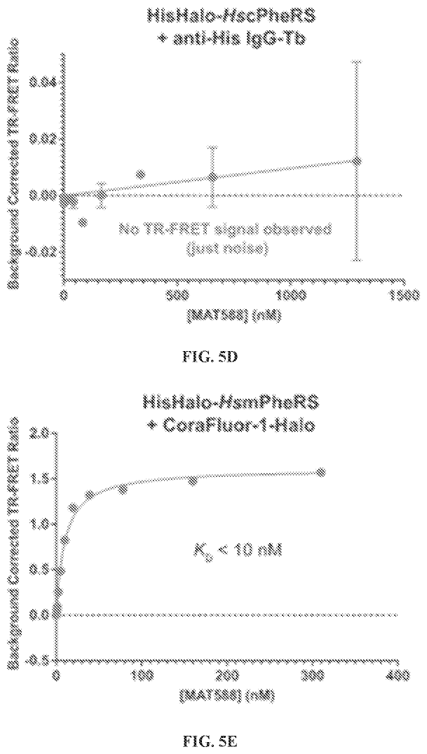

Initial batch of MAT574 was profiled by TR-FRET against both PfcProRS (K_{D}=2.27 μM) and HsProRS (K_{D}=72.2 nM) in the absence of substrates. FIG. 4 determines the affinity of proline and ATP for HsProRS using MAT574 as tracer.

MAT574 or pyrazinamide-based tracers (MAT379 and MAT425) could be used for bacterial ProRS paralogs. ProRS from P. aeruginosa (residues 10-546) and S. aureus (residues 2-567) were recombinantly expressed as N-terminal His6-ThrombinSite fusion proteins and purified. Samples of each protein were labeled with CoraFluor-1-Pfp following the same protocol used for anti-His6 IgG. The affinity of each TR-FRET tracer was then determined by TR-FRET for P. aeruginosa ProRS and S. aureus ProRS, respectively, in both the antibody format (unlabeled ProRS and two molar equivalents of CoraFluor-1-Pfp-labeled anti-His6 IgG) or with CoraFluor-1-Pfp-labeled ProRS.

In all cases. the antibody format worked. Direct chemical labeling with CoraFluor-1-Pfp worked much better and the binding affinities for tracers were found (FIG. 4). It is interesting to note that unlike for HsProRS, the pyrazinamide tracers worked better for the bacterial ProRS paralogs than MAT574. The differential affinity for MAT379 and MAT425 suggests that MAT574 analogs with different linker composition or lengths may have higher affinity. Either way. MAT425 is sufficiently high affinity for screening inhibitors, as evidenced by my success with MAT379 and HsProRS, but may benefit from using higher ProRS concentrations than were necessary for MAT379 (e.g., 25 nM). MAT574 was profiled against HsGluProRS in cell lysates.

The following hybrid tracer was also prepared for ProRS.

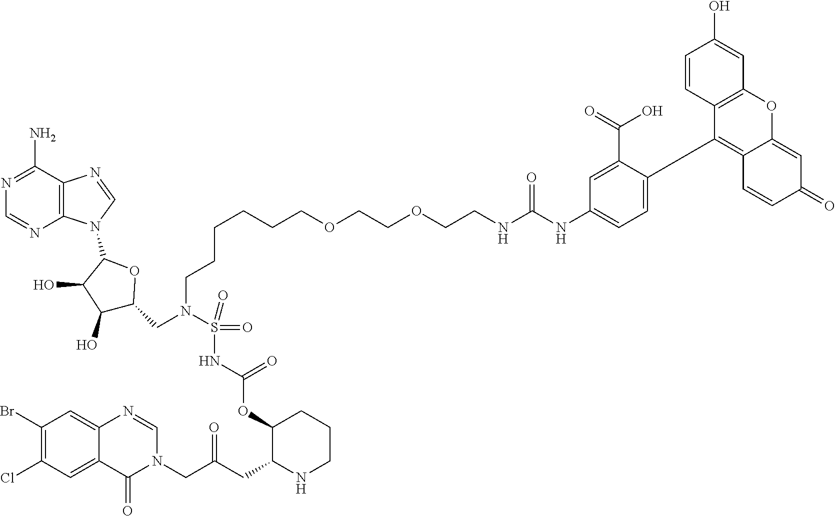

Phenylalanyl-tRNA synthetase

An assay was developed for the phenylalanyl-tRNA synthetase (PheRS). PheRS inhibition has been indicated as a promising target for the development of drugs to treat tuberculosis and malaria, where PheRS inhibitors were demonstrated to be single dose cures of the ABS. To test hypothesis that PheRS tracers analogous to MAT574 have affinity to corresponding aaRS, phenylalanyl sulfamidyladenosine (PheSNA) was prepared and the unlabeled linker-modified analog (MAT581).

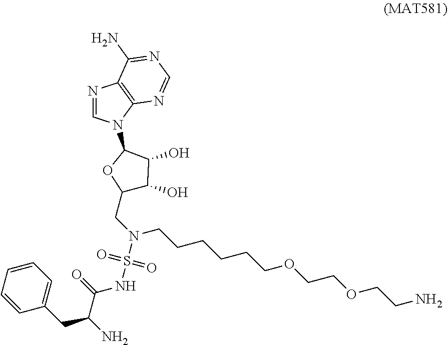 [SMILES: NCCOCCOCCCCCCN(CC1O[C@@H](n2cnc3c(N)ncnc32)[C@H](O)[C@@H]1O)S(=O)(=O)NC(=O)[C@@H](N)Cc1ccccc1]

These compounds were profiled in an activity-based assay for both MtbPheRS (Mycobacterium tuberculosis phenylalanyl-tRNA synthetase) and HscPheRS (human cytoplasmic phenylalanyl-tRNA synthetase (αβ)₂). Notably, while the enzymatic inhibition activities (not affinities) for PheSNA (MtbPheRS IC₅₀=2 μM and HscPheRS IC₅₀=0.14 μM) and MAT581 (MtbPheRS IC₅₀=206 μM and HscPheRS IC₅₀>333 M) were lower, this activity-based assay requires the use of high concentrations of both phenylalanine (100 μM) and ATP (200 μM). These concentrations are within 10-fold of the K_{D} values we determined for proline and ATP for both HsProRS and PfcProRS but phenylalanine and ATP aren't merely competitive substrates as they are also converted to phenylalanyl-AMP which is expected to have comparable affinity to PheSA and ProSA. This is consistent with ProRS TR-FRET assay data where no binding was observed with MAT379 for either HT-PfcProRS or HT-HsProRS with these simultaneous concentrations of proline and ATP, or even with >10× lower concentrations of each substrate (not to be confused with using these concentrations of each substrate separately). However, these results are also consistent with the possibility that the linker attachment site, linker composition, or linker length was less suited for PheRS than ProRS. Thus, PheSNA-derived tracer (MAT588) was prepared using the scheme described for MAT574.

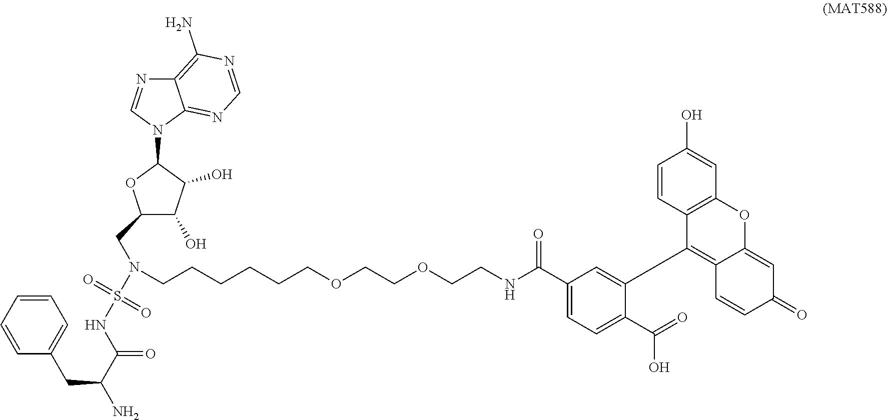 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H](N)Cc2ccccc2)[C@@H](O)[C@H]1O]

Several PheRS constructs from diverse species were expressed and purified: P. falciparum 3D7 cytoplasmic PheRS (His-PfcPheRS; alpha subunit residues 1-575; beta subunit residues 1-623 as C-terminal T4L-HRV-3C-His9 fusion), human cytoplasmic PheRS (HisHalo-HscPheRS; alpha subunit residues 1-508; beta subunit residues 1-589 as C-terminal HaloTag-ThrombinSite-His9 fusion), human cytoplasmic PheRS (His-HscPheRS: alpha subunit residues 1-508; beta subunit residues 1-589 as C-terminal HRV-3C-His9 fusion), human mitochondrial PheRS (HisHalo-HsmPheRS; residues 1-410 as C-terminal Halo-HRV-3C-His9 fusion), M. tuberculosis PheRS (HisHalo-MtbPheRS: alpha subunit residues 28-343 as N-terminal His6-ThrombinSite-HaloTag fusion; beta subunit residues 1-831), M. tuberculosis PheRS (His-MtbPheRS; alpha subunit residues 4-343 as N-terminal His6-ThrombinSite fusion; beta subunit residues 1-831), E. coli PheRS (HisHalo-EcPheRS; alpha subunit residues 7-331 as N-terminal His6-HaloTag fusion; beta subunit residues 7-795). A. thaliana cytoplasmic PheRS (His-AtcytoPheRS; alpha subunit residues 2-485 as N-terminal His9-HRV-3C fusion: beta subunit residues 1-598 as C-terminal S. pneumoniae NanA fusion (residues 296-776)), and A. thaliana chloroplast PheRS (His-AtchloroPheRS residues 54-429 as C-terminal HRV-3C-His9 fusion). Following expression, all aliquots of the His-HscPheRS and His-MtbPheRS constructs that did NOT contain HaloTag were treated with protease (HRV-3C and Thrombin, respectively) to remove the fusion tags. Aliquots of each construct were labeled with CoraFluor-1-Pfp following the same protocol as for anti-His6 IgG. Aliquots of each construct containing HaloTag were labeled with CoraFluor-1-Halo using the same protocol as for HT-ProRS.

TR-FRET assay was performed on serial dilutions of tracer MAT588. All constructs were tested as CoraFluor-1-Pfp-labeled PheRS, constructs containing a His-tag were also tested with the antibody format (unlabeled PheRS and two molar equivalents of CoraFluor-1-Pfp-labeled anti-His6 IgG), and constructs containing a HaloTag were also tested as CoraFluor-1-Halo-labeled PheRS.

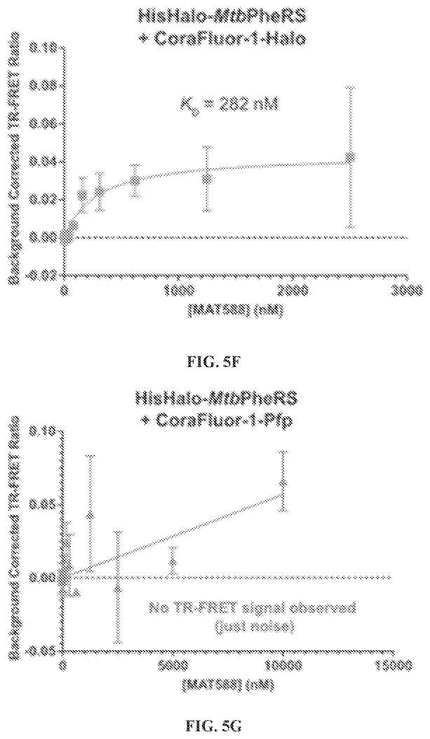

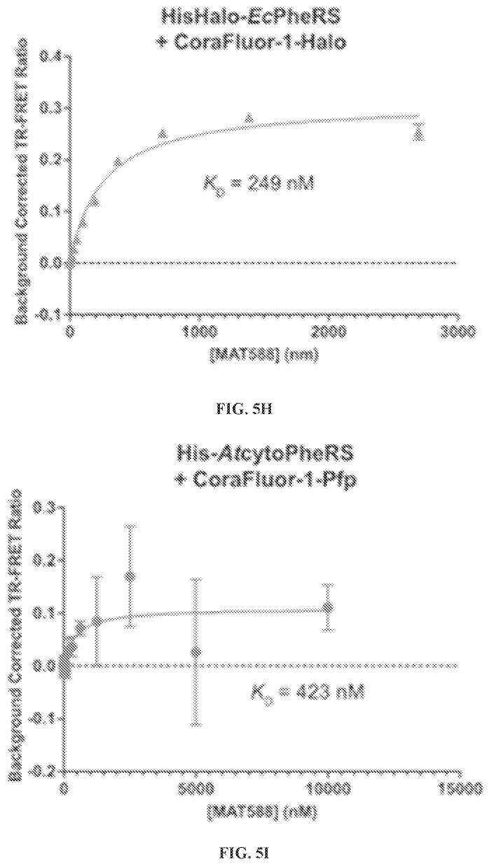

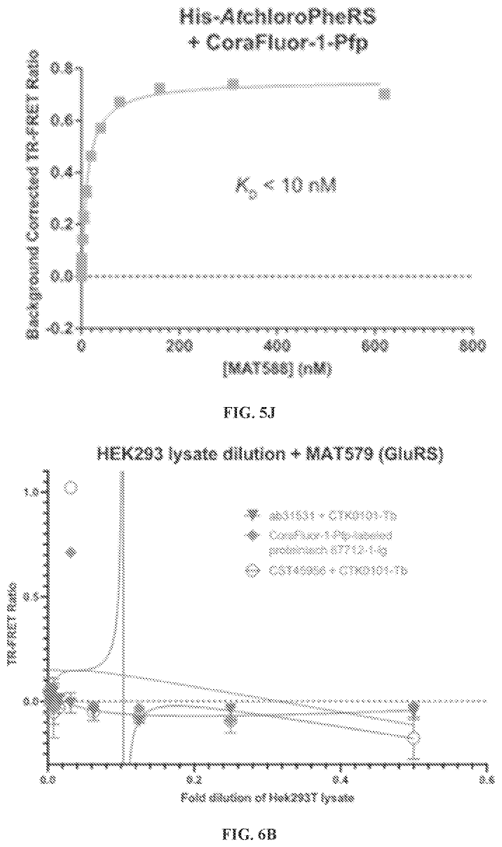

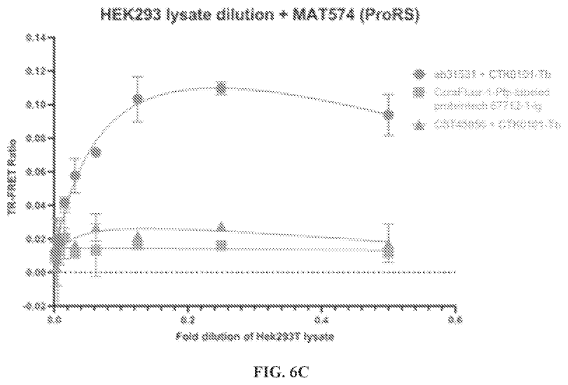

As can be seen in FIG. 5, the HaloTag system was generally the best, but the direct chemical labeling was often a viable strategy. The antibody format worked poorly, and we hypothesize that this is because the antibody put too much separation between the TR-FRET donor and acceptor. However, the direct chemical labeling worked much better, enabling the determination of equilibrium dissociation constants (K_{D}) for MAT588 (FIG. 5.5b-j). Surprisingly. MAT588 had >1,000-fold selectivity for HsmPheRS (K_{D}<10 nM) over HscPheRS (K_{D}>10 μM). As this data was only recently acquired, we have not yet had time to repeat this assay with reduced protein concentrations for the titrating paralogs (human mitochondrial PheRS and A. thaliana chloroplast PheRS).

### Isoleucyl-tRNA Synthetase

Isoleucyl sulfamidyladenosine (IleSNA) was prepared using analogous procedures, it was verified that IleSNA is active in P. falciparum (ABS IC₅₀=1.19 nM).

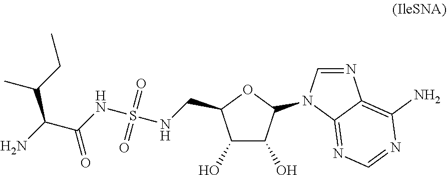

IleRS from both S. aureus (residues 1-917) and E. coli (residues 1-938) was recombinantly expressed as C-terminal His6 fusion proteins and purified. A tracer could be prepared:

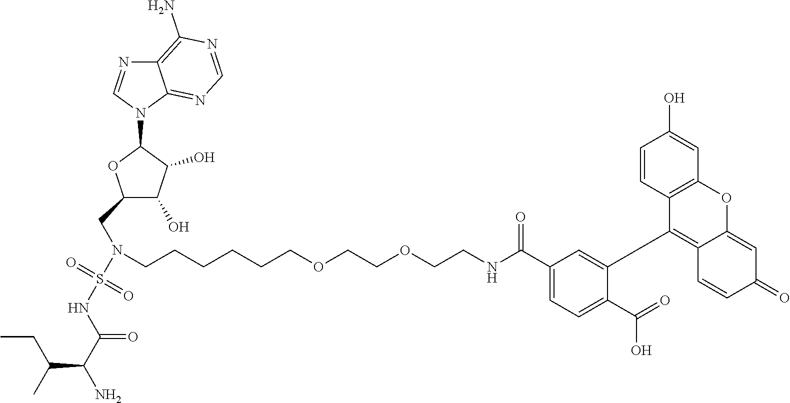 [SMILES: CCC(C)[C@H](N)C(=O)NS(=O)(=O)N(CCCCCCOCCOCCNC(=O)c1ccc(C(=O)O)c(-c2c3ccc(=O)cc-3oc3cc(O)ccc23)c1)C[C@H]1O[C@@H](n2cnc3c(N)ncnc32)[C@H](O)[C@@H]1O]

### Glutamyl-tRNA Synthetase and Human Bifunctional Glutamate Proline-tRNA Synthetase

Schemes analogous to those shown above were used to synthesize the GluSNA analogs—glutamyl sulfamidyladenosine (GluSNA) and a fluorescently-labeled GluSNA tracer (MAT579).

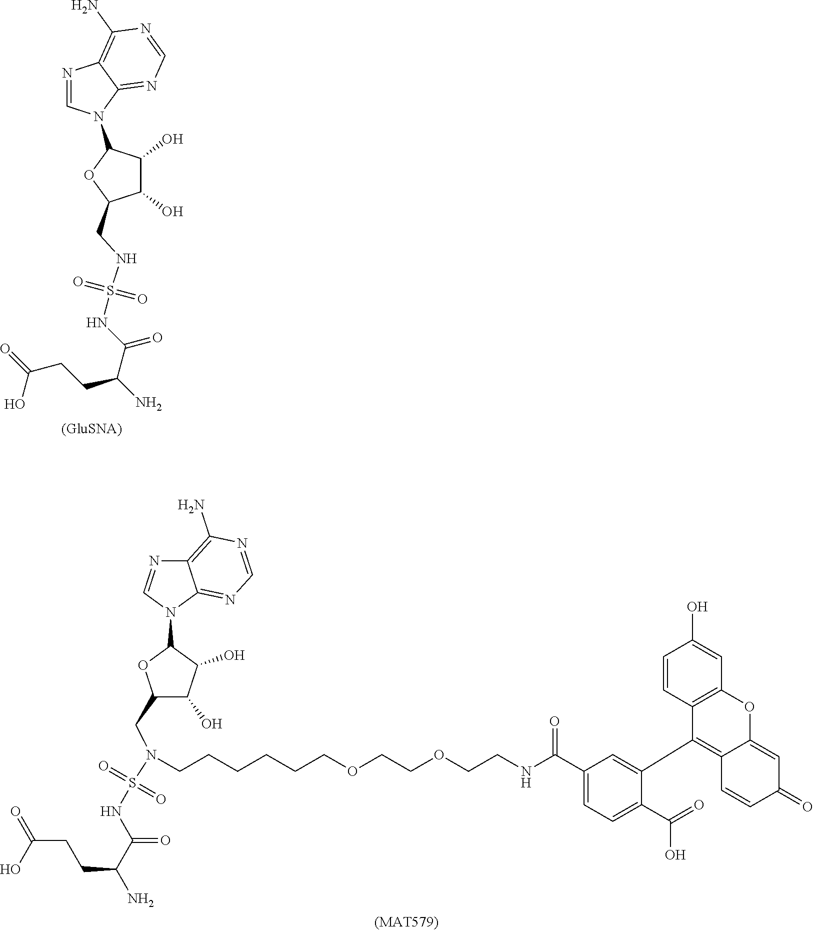

To facilitate the interrogation of basic ProRS biology, assay was developed utilizing native proteins in cell lysate or intact cells. Use of primary antibodies and CoraFluor-1-Pfp-labeled secondary nanobodies (single-domain antibodies) is as a powerful alternative to direct chemical labeling of the antibodies. Thus, three commercial anti-HsGluProRS antibodies (Abcam ab31531, Proteintech 67712-1-1g, and Cell Signaling Technology #45956) were obtained. The Proteintech 67712-1-Ig antibody was directly labeled with CoraFluor-1-Pfp. Notably though, this was not an option for the Cell Signaling Technology #45956 antibody due to the presence of 100 μg/ml BSA.

For the TR-FRET assay, HEK293T lysate was serially diluted into 384-well plates containing constant concentrations of tracer (either 250 nM MAT574 for ProRS or 250 nM MAT579 for GluRS), one of the antibodies (5 nM), and for the unlabeled antibodies, CoraFluor-1-Pfp labeled nano secondary (20 nM). No TR-FRET signal was observed for MAT579 under any conditions, whereas specific TR-FRET signal was only observed for MAT574 using the Abeam ab31531 antibody and the secondary nanobody.

### Discussion

The results show generalization of TR-FRET assay platform to aaRS enzymes from diverse bacterial and eukaryotic species. aaSNA-derived tracers generally have comparable binding characteristics to those observed for the ProRS assay with MAT379.

Antibody labeling strategy gave weaker TR-FRET signal and because there was too much separation between the TR-FRET donor and acceptor. HaloTag labeling and direct chemical labeling gave stronger specific TR-FRET signal (not to be confused with higher affinity). The fluorescein dye on the TR-FRET acceptors (tracers) may be replaced with a fluorophore with a longer Forster radius, such as Alexa Fluor 647 which has a ˜25% larger Förster radius. This could have a substantial effect because FRET (and TR-FRET) efficiency drops off proportionally to 1/1′, where r is the Förster radius.

While the affinity of MAT574 for PfcProRS was lower than expected based upon the corresponding MAT556 data, it is comparable to the affinity of MAT379 for HsProRS which was sufficient to quantify the affinity of ProSA for HsProRS (K_{D}=70.2 μM). Further, the higher affinity of MAT574 for HsProRS may prove useful for profiling the affinity of even tighter-binding inhibitors or for using reduce HsProRS concentrations. The affinity of pyrazinamide based ProRS tracers MAT379 and MAT425 suggests that the pyrazinamide series may also have potential for use as antibacterial therapies, but further studies are required. Results shown here measuring the affinity of HsProRS for proline and ATP with MAT574 are consistent with MAT379, indicating that the differential substrate affinities between HsProRS and PfcProRS are accurate and not caused by the selectivity of MAT379 for PfcProRS.

The determined affinities of our PheRS tracer MAT588 for several PheRS paralogs indicates that it would be suitable for inhibitor profiling. Further, MAT588 is a highly specific ligand for HsmPheRS (K_{D}<10 nM) over HscPheRS (K_{D}>10 μM). Our results suggest that MAT588 analogs could be valuable probes for interrogating the effects of mitochondrial PheRS inhibition. Unfortunately, this means that MAT588 is unable to be used as a TR-FRET tracer for HscPheRS. Based upon a detailed analysis of published crystal structures, the linker on MAT588 is too short to reach out of the HscPheRS active site. This indicates that analogs with a longer linker have affinity.

This approach is broadly applicable based upon results shown here. Thus, these assays accelerate the development of aaRS-targeted therapies for a wide range of diseases.

### Example 3—Preparation of Exemplified Compounds

### Reagents and Chemical Synthesis:

All reagents were purchased from Chem-Impex International Inc., Combi-Blocks Inc., Oakwood Chemical, Sigma Aldrich, Fisher Scientific International Inc., VWR International, and BioSynth CarboSynth and were used without purification. Detailed synthetic procedures can be found in Supplementary Information. Stock solutions of inhibitors were prepared at 10 mM in molecular biology grade DMSO (Sigma Aldrich). Preparation of TAMRA-Halo (55) and the TR-FRET donor CoraFluor-I reagents (CoraFluor-1-Halo and CoraFluor-1-Pfp) were reported previously. Halofuginone (1) was purchased from BioSynth CarboSynth and used without purification. Glyburide (28) was purchased from Combi-Blocks Inc. and used without further purification.

### 3-amino-N-(2,3-dihydro-1H-inden-2-yl)pyrazine-2-carboxamide (45)

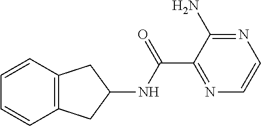 [SMILES: Nc1nccnc1C(=O)NC1Cc2ccccc2C1]

Under an argon atmosphere, mixed 3-aminopyrazine-2-carboxylic acid (7.5 g, 54 mmol, 1 eq). DCM (600 mL). and DIPEA (28 mL, 21 g, 160 mmol, 3 eq). Added isobutyl chloroformate (7.8 mL, 8.1 g, 59 mmol, 1.1 eq) and stirred 16 h. Added 2,3-dihydro-1H-inden-2-amine (9.2 g, 54 mmol, 1 eq) and stirred for 24 h. Diluted with 150 mL DCM and 5 mL MeOH. Washed sequentially with 1:1 saturated NaHCO_{3(aq)}H₂O (2×300 mL) and 1:1 saturated NaCl_{(aq)}/H₂O (1×300 mL), keeping the emulsion with the organic layer each time. Filtered insoluble (largely product, but not pure) and set filtrand aside, dried filtrate over anhydrous Na₂SO₄, filtered, and concentrated under reduced pressure. Recombined the concentrated material with the filtrand from before. Triturated with 50 mL MeCN (note: sonicated vigorously until the solid was visually homogeneous beige, the MeCN was orange, and no black/brown spots were observed; typically ˜10-15 min) and filtered. Yield: 11.1 g, 80.7%. Beige solid. ¹H NMR (400 MHz, DMSO-d₆) δ 8.81 (d, J=7.9 Hz, 1H), 8.20 (d, J=2.1 Hz, 1H), 7.80 (d, J=2.1 Hz, 1H), 7.55 (s, 2H), 7.22 (dd. J=5.5, 3.3 Hz, 2H), 7.15 (dd, J=5.4, 3.2 Hz, 2H), 4.70 (h, J=7.5 Hz, 1H), 3.17 (dd, J=15.7, 7.6 Hz, 2H), 3.01 (dd, J=15.7, 7.3 Hz, 2H). ¹C NMR (101 MHz, DMSO-d₆) δ 165.83, 155.19, 146.83, 141.18, 130.81, 126.46, 125.65, 124.47, 50.23, 38.59. LC-MS (C₁₄H₁₄N₄O): Calculated [M+H]⁺ m/z=255.12. Observed [M+H]⁺ m/z 255.33.

### tert-butyl (1-((3-((2,3-dihydro-1H-inden-2-yl)carbamoyl)pyrazin-2-yl)carbamoyl)piperidin-4-yl)carbamate (7)

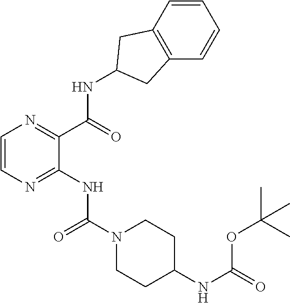 [SMILES: CC(C)(C)OC(=O)NC1CCN(C(=O)Nc2nccnc2C(=O)NC2Cc3ccccc3C2)CC1]

To a solution of 45 (2.00 g, 7.87 mmol, 1 eq) in anhydrous DCM (300 mL), added anhydrous lutidine (3.64 mL, 3.37 g, 31.5 mmol, 4 eq). Cooled to 0° C. Added 15% w/v phosgene₍ₜₒₗᵤₑₙₑ₎ (7.86 mL, 11.0 mmol, 1.4 eq) slowly along the walls over 5 s min, removed from 0° C. bath, and stirred for 1 h. Added a solution of tert-butyl piperidin-4-ylcarbamate (3.15 g, 15.7 mmol, 2 eq) in DCM (125 mL) and stirred at room temperature overnight. Quenched reaction mixture with MeOH and concentrated reaction mixture to dryness. Purified by normal phase flash column chromatography (hexanes/EtOAc). Yield: 776 mg, 20.5%. White solid. ¹H NMR (400 MHz, CDCl₆) δ 11.49 (s, 1H), 8.52 (d, J=2.2 Hz, 1H), 8.28 (d, J=8.2 Hz, 1H), 8.01 (d, J=2.3 Hz, 1H), 7.31-7.24 (m, 2H), 7.24-7.18 (m, 2H), 4.93-4.79 (m, 1H), 4.46 (s, 1H), 4.23 (d, J=13.6 Hz, 2H), 3.70 (s, 1H), 3.42 (dd, J=16.2, 7.2 Hz, 2H), 3.08 (t, J=12.6 Hz, 2H), 2.96 (dd, J=16.1, 4.9 Hz, 2H), 2.04 (d, J=11.3 Hz, 2H), 1.45 (d, J=7.6 Hz, 9H), 1.39 (dd, J=12.1, 4.2 Hz, 2H). ¹³C NMR (101 MHz, CDCl₃) δ 165.89, 155.20, 152.59, 151.20, 146.78, 140.63, 135.06, 128.69, 127.13, 124.99, 79.74, 50.68, 48.00, 43.27, 40.12, 32.59, 28.55. LC-MS (C₂₅H₃₂N₆O₄): Calculated [M+H]⁺ m/z=481.26, [M−H]⁻ m/z=479.24. Observed [M+H]⁺ m/z=481.39, [M−H]⁻ m/z=479.40.

### 3-(4-aminopiperidine-1-carboxamido)-N-(2,3-dihydro-1H-inden-2-yl)pyrazine-2-carboxamide dihydrochloride (8-2HCl)

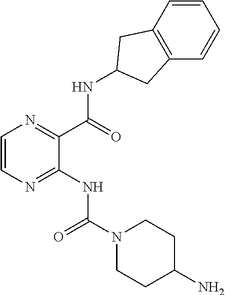 [SMILES: NC1CCN(C(=O)Nc2nccnc2C(=O)NC2Cc3ccccc3C2)CC1]

Charged a flask with 7 (2.00 g, 4.16 mmol, 1 eq) and methanol (10 mL). Added 4.0 M HCl_{(1,4-dioxane)} (10 mL) and stirred vigorously for 1.75 h. Concentrated in vacuo. Yield: 1.85 g, >95%. White solid. ¹H NMR (400 MHz, DMSO) δ 11.04 (s, 1H), 9.21 (d, J=7.8 Hz, 1H), 8.51 (d, J=2.4 Hz, 1H), 8.43 (d, J=5.2 Hz, 3H), 8.23 (d, J=2.4 Hz, 1H), 7.27-7.16 (m, 2H), 7.18-7.05 (m, 2H), 4.71 (h, J=7.6 Hz, 1H), 4.08 (d, J=13.8 Hz, 2H), 3.28 (tt, J=10.6, 5.0 Hz, 1H), 3.19 (dd, J=15.8, 7.8 Hz, 2H), 3.09-2.94 (m, 4H), 2.08-1.93 (m, 2H), 1.55 (qd, J=12.3, 4.2 Hz, 2H). ¹³C NMR (101 MHz, DMSO) δ 165.66, 152.29, 149.82, 145.63, 141.13, 135.71, 130.75, 126.51, 124.50, 50.48, 47.47, 42.11, 38.48, 29.56. LC-MS (C₂₀H₂₄N₆O₂): Calculated [M+H]⁺ m/z=381.20, [M−H]⁻ m/z=379.19. Observed [M+H]⁺ m/z=381.44, [M−H]⁻ m/z=379.43.

### tert-butyl ((1-((3-((2,3-dihydro-1H-inden-2-yl)carbamoyl)pyrazin-2-yl)carbamoyl)piperidin-4-yl)methyl)carbamate (9)

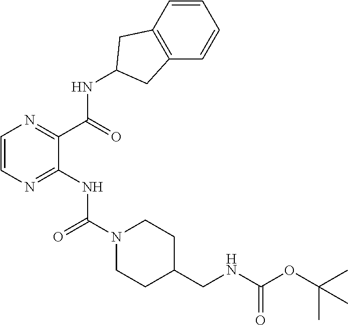 [SMILES: CC(C)(C)OC(=O)NCC1CCN(C(=O)Nc2nccnc2C(=O)NC2Cc3ccccc3C2)CC1]

Charged a flask with 45 (2.99 g, 11.8 mmol, 1 eq), DCM (400 mL), and 2,6-lutidine (6.83 mL, 6.32 g, 59.0 mmol, 5 eq). Cooled reaction to 0° C. Added 15% w/v phosgene₍ₜₒₗᵤₑₙₑ₎ (12 mL, 16.8 mmol, 1.4 eq), removed reaction from 0° C. bath, and let stir at room temperature for 1 h. During this time, charged a second flask with tert-butyl (piperidin-4-ylmethyl)carbamate (4.00 g, 18.7 mmol, 1.59 eq) and DCM (100 mL). Transferred the contents of the first flask to the second flask and stirred vigorously for 20 h. Quenched with methanol, added granular silica (˜15 g), and concentrated to dryness. Purified by normal phase flash column chromatography (hexanes/EtOAc). Yield: 1.14 g, 19.7%. White solid. ¹H NMR (400 MHz, CDCl₃) δ 11.46 (s, 1H), 8.52 (d, J=2.3 Hz, 1H), 8.28 (d, J=8.1 Hz, 1H), 8.00 (d, J=2.3 Hz, 11H), 7.30-7.24 (m, 2H), 7.24-7.15 (m, 2H), 4.92-4.80 (m, 1H), 4.65 (t, J=6.1 Hz, 1H), 4.32 (d, J=13.3 Hz, 2H), 3.42 (dd, J=16.2, 7.2 Hz, 2H), 3.05 (t, J=6.3 Hz, 2H), 3.01-2.86 (m, 4H), 1.79 (d, J=13.3 Hz, 2H), 1.75-1.64 (m, 1H), 1.44 (s, 9H), 1.25 (qd, J=12.2, 4.3 Hz, 2H). ¹³C NMR (101 MHz, CDCl₃) δ 165.89, 156.18, 152.59, 151.24, 146.75, 140.65, 134.92, 128.61, 127.10, 124.97, 79.48, 53.57, 51.02, 50.65, 46.05, 44.29, 41.07, 40.11, 36.91, 29.78, 28.54. LC-MS (C₂₆H₃₄N₅O₄): Calculated [M+H]⁺ m/z=495.27, [M−H]⁻ m/z=493.26. Observed [M+H]⁺ m/z=495.67, [M−H]⁻ m/z=493.45.

### 3-(4-(aminomethyl)piperidine-1-carboxamido)-N-(2,3-dihydro-1H-inden-2-yl)pyrazine-2-carboxamide dihydrochloride (10·2HCl)

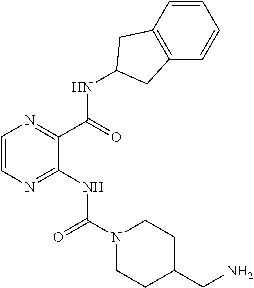 [SMILES: NCC1CCN(C(=O)Nc2nccnc2C(=O)NC2Cc3ccccc3C2)CC1]

Diluted 4.0 M HCl(1,4-dioxane) (4 mL) with methanol (12 mL) and stirred until mixture cooled to room temperature. Added this to a vial containing 9 (799 mg, 1.62 mmol, 1 eq) and stirred vigorously for 2 h. Concentrated in vacuo. Yield: 797.9 mg, 93.2%. White solid. 1H NMR (400 MHz, DMSO) δ 11.01 (s, 1H), 9.22 (d, J=7.8 Hz, 1H), 8.51 (d, J=2.4 Hz, 1H), 8.22 (d, J=2.3 Hz, 1H), 8.03 (s, 3H), 7.27-7.19 (m, 2H), 7.19-7.09 (m, 2H), 4.71 (h, J=7.5 Hz, 1H), 4.07 (d. J=13.4 Hz, 2H), 3.24-3.14 (m, 2H), 3.06 (dd, J=15.8, 7.3 Hz, 2H), 2.91 (t, J=12.7 Hz, 2H), 2.73 (h, J=6.0 Hz, 2H), 1.92-1.83 (m, 1H), 1.83-1.73 (m, 2H), 1.19 (qd, J=12.3, 4.0 Hz, 2H). 13C NMR (101 MHz, DMSO) δ 165.68, 152.16, 149.96, 145.72, 141.11, 135.52, 130.50, 126.49, 124.47, 50.45, 43.48, 38.43, 33.86, 28.91. LC-MS (C21H26N6O2): Calculated [M+H]+ m/z=395.22, [M−H]− m/z=393.20. Observed [M+H]+ m/z=395.04, [M−H]− m/z=393.36.

### tert-butyl (S)-2-((1-((3-((2,3-dihydro-1H-inden-2-yl)carbamoyl)pyrazin-2-yl)carbamoyl)piperidin-4-yl)carbamoyl)pyrrolidine-1-carboxylate (31)

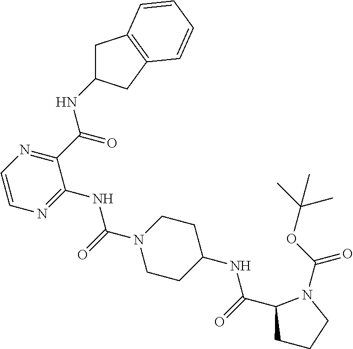 [SMILES: CC(C)(C)OC(=O)N1CCC[C@H]1C(=O)NC1CCN(C(=O)Nc2nccnc2C(=O)NC2Cc3ccccc3C2)CC1]

Charged a vial with 8·2HCl (185 mg, 408 μmol, 1 eq), Boc-L-proline N-hydroxysuccinimide ester (199 mg, 636 μmol, 1.56 eq), DCM (5 mL), and DIPEA (400 μL, 297 mg, 2.30 mmol, 5.6 eq). Stirred 3 h. Concentrated reaction mixture in vacuo to a white solid. Purified by normal phase flash column chromatography (DCM/MeOH). Yield: 216 mg, 91.4%. White solid. ¹H NMR (400 MHz, DMSO) δ 11.01 (s, 1H), 9.21 (d, J=7.8 Hz, 1H), 8.50 (d, J=2.4 Hz, 1H), 8.22 (d, J=2.4 Hz, 1H), 7.91-7.74 (m, 1H), 7.28-7.18 (m, 2H), 7.19-7.09 (m, 2H), 4.72 (h, J=7.6 Hz, 1H), 4.08-3.90 (m, 3H), 3.90-3.73 (m, 1H), 3.43-3.29 (m, 1H), 3.31-3.21 (m, 1H), 3.19 (dd. J=15.8, 7.7 Hz, 2H), 3.04 (s, 4H), 2.16-2.00 (m, 1H), 1.87-1.66 (m, 5H), 1.50-1.24 (m, 11H). ¹³C NMR (101 MHz, DMSO) δ 171.72, 165.66, 153.30, 152.20, 149.97, 145.70, 141.10, 135.51, 130.55, 126.46, 124.44, 78.48, 78.32, 59.68, 54.93, 50.44, 46.51, 45.69, 42.89, 38.42, 31.53, 31.40, 31.22, 28.14, 28.09, 23.18. LC-MS (C₃₀H₃₉N₇O₅): Calculated [M+H]⁺ m/z=578.31, [M−H]⁻ m/z=576.29. Observed [M+H]⁺ m/z=578.63, [M−H]⁻ m/z=576.58.

### (S)-N-(2,3-dihydro-1H-inden-2-yl)-3-(4-(pyrrolidine-2-carboxamido)piperidine-1-carboxamido)pyrazine-2-carboxamide (MAT334, 29)

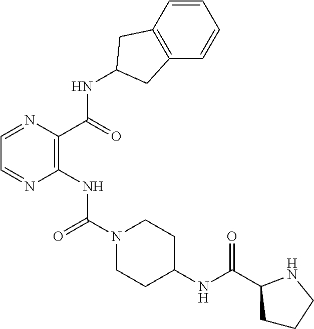 [SMILES: O=C(NC1Cc2ccccc2C1)c1nccnc1NC(=O)N1CCC(NC(=O)[C@@H]2CCCN2)CC1]

Diluted 4.0 M HCl_{(1,4-dioxane)} (1 mL) with methanol (3 mL) and stirred until mixture cooled to room temperature. Added this to a vial containing 31 (77.5 mg, 134 μmol, 1 eq) and stirred vigorously for 2.5 h. Concentrated to a light brown oil. Purified by reverse phase flash column chromatography and eluted with water+0.1% formic acid/MeCN+0.1% formic acid. Concentrated to dryness and azeotroped with methanol (2 mL). Yield: 57.1 mg, 89.1%. White solid. ¹H NMR (400 MHz, DMSO) δ 11.00 (s, 1H), 9.21 (d, J=7.8 Hz, 1H), 8.50 (d, J=2.5 Hz, 1H), 8.41 (d, J=7.7 Hz. 1H), 8.28 (s, 1H), 8.22 (d, J=2.4 Hz, 1H), 7.25-7.17 (m, 2H), 7.19-7.10 (m, 2H), 4.72 (h, J=7.6 Hz, l H), 3.98 (dd, J=12.9, 4.8 Hz, 3H), 3.92-3.76 (m, 1H), 3.26-3.14 (m, 2H), 3.14-2.96 (m, 6H), 2.23-2.10 (m, 1H), 1.88-1.69 (m, 5H), 1.51-1.34 (m, 2H). ¹³C NMR (101 MHz, DMSO) δ 169.37, 165.67, 152.30, 149.93, 145.68, 141.11, 135.56, 130.68, 126.48, 124.46, 59.12, 50.45, 46.00, 45.77, 42.64, 38.44, 31.20, 30.03, 24.29. LC-MS (C₂₅H₃₁N₇O₃): Calculated [M+H]⁺ m/z=478.26, [M−H]⁻ m/z=476.24. Observed [M+H]⁺ m/z=478.55, [M−H]⁻ m/z=476.55.

### tert-butyl (S)-2-(((1-((3-((2,3-dihydro-1H-inden-2-yl)Carbamoyl)pyrazin-2-yl)carbamoyl)piperidin-4-yl)methyl)carbamoyl)pyrrolidine-1-carboxylate (32)

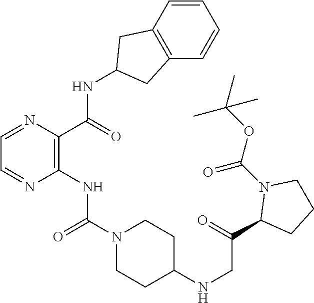 [SMILES: CC(C)(C)OC(=O)N1CCC[C@H]1C(=O)CNC1CCN(C(=O)Nc2nccnc2C(=O)NC2Cc3ccccc3C2)CC1]

Charged a vial with 10 (141 mg, 356 μmol, 1 eq), Boc-L-proline N-hydroxysuccinimide ester (152 mg, 488 μmol, 1.37 eq), DCM (5 mL), and DIPEA (400 μL, 297 mg, 2.30 mmol, 6.45 eq). Stirred 3 h. Concentrated reaction mixture in vacuo to a white solid. Purified by normal phase flash column chromatography (DCM/MeOH). Yield: 153 mg, 72.5%. White solid. ¹H NMR (400 MHz, DMSO) δ 11.02 (s, 1H), 9.21 (d, J=7.8 Hz, 1H), 8.50 (d, J=2.4 Hz, 1H), 8.21 (d, J=2.3 Hz, 1H), 7.99-7.80 (m, 1H), 7.27-7.18 (m, 2H), 7.19-7.09 (m, 2H), 4.71 (h, J=7.6 Hz, 1H), 4.13-3.95 (m, 3H), 3.44-3.29 (m, 1H), 3.26 (dt, J=10.2, 6.8 Hz, 1H), 3.19 (dd, J=15.8, 7.7 Hz, 2H), 3.11-3.00 (m, 3H), 2.98-2.80 (m, 3H), 2.15-1.99 (m, 1H), 1.87-1.61 (m, 6H), 1.35 (d, 9H), 1.20-1.02 (m, 2H). ¹³C NMR (101 MHz, DMSO) δ 172.52, 172.15, 165.69, 153.38, 152.07, 150.05, 145.74, 141.09, 135.38, 130.30, 126.46, 124.45, 78.54, 78.37, 59.88, 50.46, 46.49, 43.74, 38.41, 35.97, 31.21, 30.71, 29.46, 28.12, 28.04, 23.98, 23.16. LC-MS (C₃₁H₄₁N₇O₅): Calculated [M+H]⁺ m/z=592.32, [M−H]⁻ m/z=590.31. Observed [M+H]⁻ m/z=592.65, [M−H]⁻ m/z=590.60.

### (S)-N-(2,3-dihydro-1H-inden-2-y)-3-(4-((pyrrolidine-2-carboxamido)methyl)piperidine-1-carboxamido)pyrazine-2-carboxamide (MA7T345, 30)

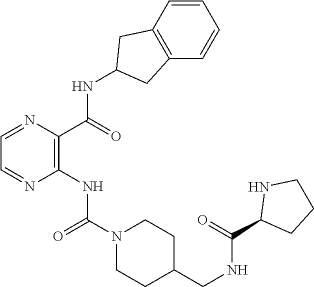 [SMILES: O=C(NC1Cc2ccccc2C1)c1nccnc1NC(=O)N1CCC(CNC(=O)[C@@H]2CCCN2)CC1]

Diluted 4.0 M HCl_{(1,4-dioxane)} (1 mL) with methanol (3 mL) and stirred until mixture cooled to room temperature. Added this to a vial containing 32 (78.1 mg, 132 μmol, 1 eq) and stirred vigorously for 2.5 h. Concentrated to a light brown oil. Purified by reverse phase flash column chromatography and eluted with water+0.1% formic acid/MeCN+0.1% formic acid. Concentrated to dryness and azeotroped with methanol (2 mL). Yield: 60.8 mg, 93.7%. White solid. ¹H NMR (400 MHz, DMSO) δ 11.02 (s, 1H), 9.22 (d, J=7.8 Hz, 1H), 8.50 (d, J=2.4 Hz, 1H), 8.34 (t, J=5.9 Hz, 1H), 8.28 (s, 1H), 8.21 (d, J=2.4 Hz, 1H), 7.25-7.18 (m, 2H), 7.18-7.11 (m, 2H), 4.71 (h, J=7.6 Hz, 1H), 4.06 (d, J=13.1 Hz, 2H), 3.93-3.82 (m, 1H), 3.19 (dd, J=15.8, 7.7 Hz, 2H), 3.10-2.97 (m, 6H), 2.88 (t, J=12.6 Hz, 2H), 2.19-2.03 (m, 1H), 1.82-1.60 (m, 6H), 1.19-1.05 (m, 2H). ¹³C NMR (101 MHz, DMSO) δ 171.11, 165.69, 152.12, 150.05, 145.76, 141.10, 135.42, 130.29, 126.48, 124.46, 59.42, 50.45, 45.93, 43.78, 38.43, 35.79, 30.15, 29.39, 24.65. LC-MS (C₂₆H₃₃N₇O₃): Calculated [M+H]⁺ m/z=492.27, [M−H]⁻ m/z=490.26. Observed [M+H]⁺ m/z=492.14, [M−H]⁻ m/z=490.56.

### tert-butyl (6-((1-((3-((2,3-dihydro-1H-inden-2-yl)carbamoyl)pyrazin-2-yl)carbamoyl)piperidin-4-yl)amino)-6-oxohexyl)carbamate (41)

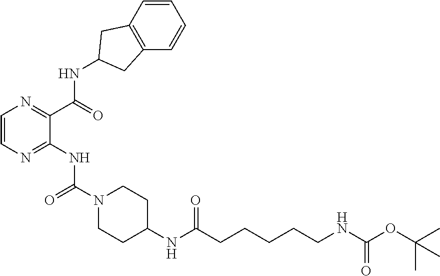 [SMILES: CC(C)(C)OC(=O)NCCCCCC(=O)NC1CCN(C(=O)Nc2nccnc2C(=O)NC2Cc3ccccc3C2)CC1]

To a stirred solution of 8·2HCl (213 mg, 470 μmol, 1 eq), DCM (5 mL), and DIPEA (307 μL, 228 mg, 1.76 mmol, 3.47 eq), added a solution of 51 (214.5 mg, 540 μmol, 1.15 eq) in DCM (5 mL) and stirred 4 h. Diluted with EtOAc (300 mL)+MeOH (3 mL), washed twice with 1:1 mixture of water and saturated NaHCO_{3(aq)} (200 mL), washed twice with 0.2M HCl_{(aq)} (50 mL), and washed once with 3:1 mixture of water and saturated NaCl_{(aq)} (100 mL). Dried over Na₂SO₄, filtered, and concentrated in vacuo. Purified by reverse phase flash column chromatography (water+0.1% formic acid/MeCN+0.1% formic acid). Yield: 254 mg, 88.9%. White solid. ¹H NMR (400 MHz, CDCl₃) δ 11.51 (s, 1H), 8.49 (d, J=2.4 Hz, 1H), 8.28 (d, J=8.2 Hz, 1H), 8.02 (d, J=2.4 Hz, 1H), 7.29-7.16 (m, 4H), 5.63 (d, J=8.0 Hz, 1H), 4.85 (dtd, J=12.3, 7.4, 4.9 Hz, 1H), 4.56 (s, 1H), 4.25 (d, J=13.6 Hz, 2H), 4.03 (dtt, J=11.6, 7.9, 3.9 Hz, 1H), 3.41 (dd, J=16.1, 7.2 Hz, 2H), 3.14-3.02 (m, 4H), 2.95 (dd, J=16.1, 4.8 Hz, 2H), 2.17 (t, J=7.6 Hz, 2H), 2.02 (dd, J=13.2, 3.9 Hz, 2H), 1.65 (p, J=7.6 Hz, 2H), 1.55-1.25 (m, 15H). ¹³C NMR (101 MHz, CDCl₃) 172.73, 165.72, 156.21, 152.57, 150.85, 146.15, 140.56, 135.12, 128.93, 127.12, 124.96, 79.34, 50.71, 46.73, 43.37, 40.58, 40.07, 36.53, 32.13, 29.83, 28.55, 26.41, 25.42. LC-MS (C₃₁H₄₃N₇O₅): Calculated [M+H]⁺ m/z=594.73, [M−H]⁻ m/z=592.73. Observed [M+H]⁺ m/z=594.66, [M−H]⁻ m/z=592.56.

### 3-(4-(6-aminohexanamido)piperidine-1-carboxamido)-N-(2,3-dihydro-1H-inden-2-y-l)pyrazine-2-carboxamide dihydrochloride (42·2HCl)

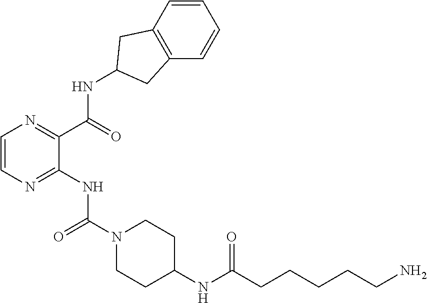 [SMILES: NCCCCCC(=O)NC1CCN(C(=O)Nc2nccnc2C(=O)NC2Cc3ccccc3C2)CC1]

To a vigorously stirred solution of MeOH (6 mL), added SOCl₂ (500 μL, 815 mg, 6.85 mmol, 35.5 eq) dropwise over 5 minutes and stirred for 20 minutes. Added the entire MeOH/SOCl₂ solution to dry 41 (115 mg, 193 μmol, 1 eq) and stirred for 45 min. Concentrated to a dry white solid. Yield: 108.3 mg, >95%. White solid. ¹H NMR (400 MHz, MeOD) δ 8.43 (s, 2H), 7.29-7.11 (m, 4H), 4.85 (t, J=6.6 Hz, 1H), 4.18 (d, J=13.0 Hz, 2H), 3.99 (s, 1H), 3.39-3.32 (m, 2H), 3.22 (t, J=12.1 Hz, 2H), 3.07 (dd, J=15.8, 6.4 Hz, 2H), 2.93 (t, J=7.2 Hz, 2H), 2.25 (t, J=7.1 Hz, 2H), 1.99 (d, J=11.4 Hz, 2H), 1.78-1.62 (m, 4H), 1.62-1.48 (m, 2H), 1.43 (q, J=7.5 Hz, 2H). ¹³C NMR (101 MHz, MeOD) δ 175.26, 166.17, 154.03, 148.65, 141.91, 139.39. 136.96, 134.29, 127.86, 125.56, 52.36, 47.58, 44.25, 40.56, 39.87, 36.57, 32.42, 28.22, 26.92, 26.31. LC-MS (C₂₆H₃₅N₇O₃): Calculated [M+H]⁺ m/z=494.29, [M−H]⁻ m/z=492.27. Observed [M+H]⁺ m/z=494.48, [M−H]⁻ m/z=492.44.

### tert-butyl (6-(((1-((3-((2,3-dihydro-1H-inden-2-yl)carbamoyl)pyrazin-2-yl)carbamoyl)piperidin-4-yl)methyl)amino)-6-oxohexyl)carbamate (43)

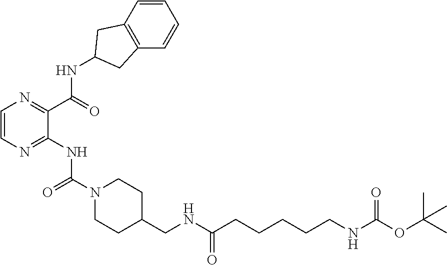 [SMILES: CC(C)(C)OC(=O)NCCCCCC(=O)NCC1CCN(C(=O)Nc2nccnc2C(=O)NC2Cc3ccccc3C2)CC1]

Charged a vial with 10·2HCl (204 mg, 437 μmol, 1 eq), DCM (5 mL), and DIPEA (298 μL, 221 mg, 1.71 mmol, 3.92 eq) and stirred vigorously. Over the course of 1 min, slowly added solution of 51 (215 mg, 540 μmol, 1.24 eq) and DCM (5 mL). Stirred 3 h. Diluted with EtOAc (300 mL) and MeOH (3 mL). Washed twice with 1:1 water/saturated NaHCO_{3(aq)} (200 mL), washed twice with 0.2M HCl_{(aq)} (50 mL), and washed once with 3:1 water/saturated NaCl(aq) (100 mL). Dried over Na₂SO₄, filtered, and concentrated in vacuo. Purified by reverse phase flash column chromatography (water+0.1% formic acid/MeCN+0.1% formic acid). Yield: 247 mg, 93%. Light yellow solid. ¹H NMR (400 MHz, CDCl₃) δ 11.45 (s, 1H), 8.49 (d, J=2.3 Hz, 1H), 8.28 (d, J=8.1 Hz, 1H), 7.99 (d, J=2.3 Hz, 1H), 7.29-7.16 (m, 4H), 5.79 (s, 1H), 4.85 (dtd, J=12.3, 7.5, 4.9 Hz, 1H), 4.58 (s, 1H), 4.30 (d, J=13.3 Hz, 2H), 3.41 (dd, J=16.2, 7.2 Hz, 2H), 3.17 (t, J=6.1 Hz, 2H), 3.09 (t, J=7.1 Hz, 2H), 3.01-2.81 (m, 4H), 2.18 (t, J=7.6 Hz, 2H), 1.85-1.72 (m, 3H), 1.65 (p, J=7.6 Hz. 2H), 1.53-1.18 (m, 15H). ¹³C NMR (101 MHz, CDCl₃) δ 173.43, 165.80, 156.18, 152.55, 151.09, 146.47, 140.60, 134.93, 128.70, 127.08, 124.95, 79.27, 50.66, 44.89, 44.25, 40.50, 40.06, 36.55, 36.41, 29.87, 28.54, 26.49, 25.46. LC-MS (C₃₂H₄₅N₇O₅): Calculated [M+H]⁺ m/z=608.76, [M−H]⁻ m/z=606.76. Observed [M+H]⁺ m/z=608.37, [M−H]⁻ m/z=606.49.

### 3-(4-((6-aminohexanamido)methyl)piperidine-1-carboxamido)-N-(2,3-dihydro-1H-inden-2-yl)pyrazine-2-carboxamide dihydrochloride (44·2HCl)

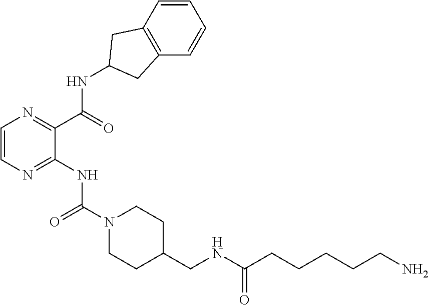 [SMILES: NCCCCCC(=O)NCC1CCN(C(=O)Nc2nccnc2C(=O)NC2Cc3ccccc3C2)CC1]

To a vigorously stirred solution of MeOH (6 mL), added SOCl₂ (500 μL, 815 mg, 6.85 mmol, 49.2 eq) dropwise over 5 minutes and stirred for 20 minutes. Added the entire MeOH/SOCl₂ solution to dry 43 (84.6 mg, 139 μmol, 1 eq) and stirred for 45 min. Concentrated to a dry white solid. Yield: 69.1 mg, 85.5%. Light yellow solid. ¹H NMR (400 MHz, MeOD) δ 8.50 (s, 1H), 8.41 (s, 1H), 7.28-7.10 (m, 4H), 4.86 (d. J=9.1 Hz, 1H), 4.23 (s, 2H), 3.40-3.32 (m, 2H), 3.19-3.04 (m, 6H), 2.92 (d, J=7.4 Hz, 2H), 2.27 (d, J=7.1 Hz, 2H), 1.87 (d, J=11.1 Hz, 3H), 1.68 (q, J=7.8 Hz, 4H), 1.43 (t, J=7.4 Hz, 2H), 1.37-1.18 (m, 2H). ¹³C NMR (101 MHz, MeOD) δ 176.15, 165.79, 153.92, 147.86, 141.89, 136.83, 135.40, 127.88, 127.82, 125.57. 52.42, 45.45, 40.57, 39.82 (s, 2C), 37.22, 36.56, 30.74, 28.22, 27.00, 26.39. LC-MS (C₂₇H₃₉C₂N₇O₃): Calculated [M+H]⁺ m/z=581.56, [M−H]⁻ m/z=579.56. Observed [M+H]⁻ m/z=508.47, [M−H]⁻ m/z=506.46.

The following compounds were obtained in a manner that is similar to making compounds 41-44:

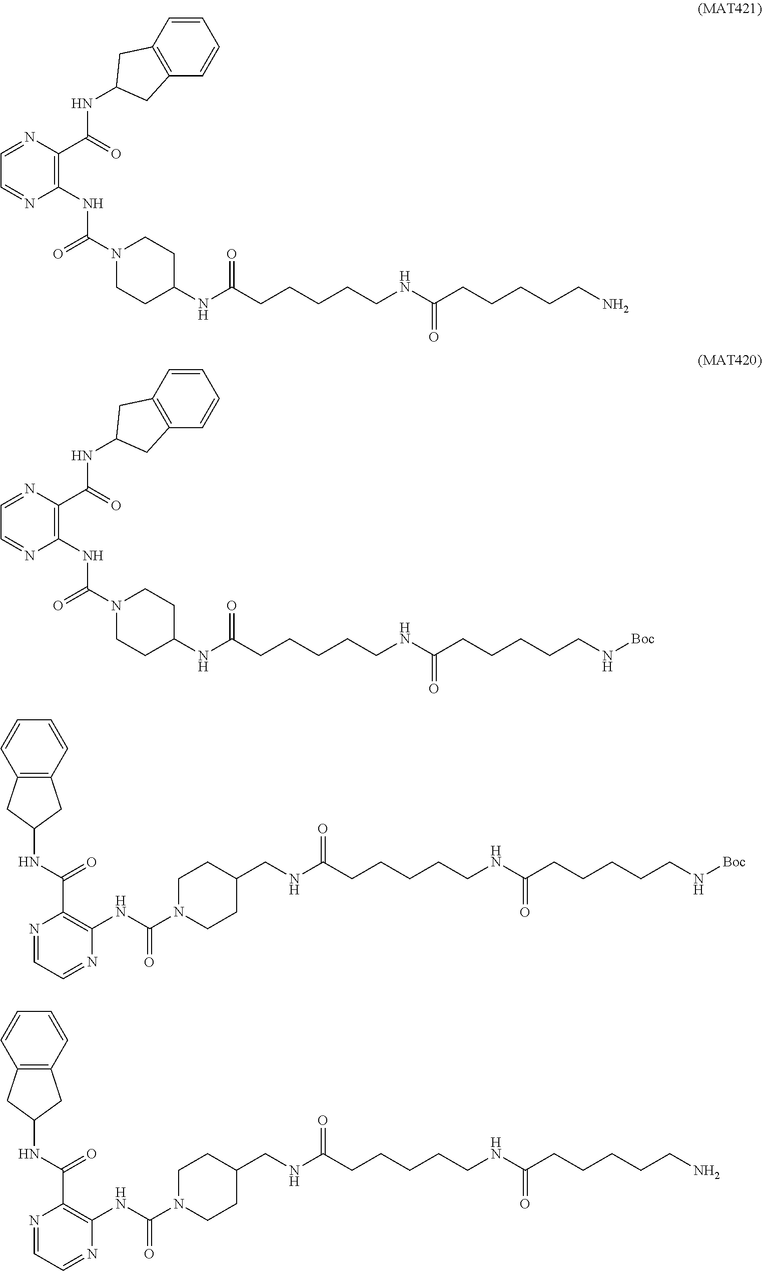

### 5-(3-((1-((3-((2,3-dihydro-1H-inden-2-yl)carbamoyl)pyrazin-2-yl)carbamoyl)piperidin-4-yl)amino)-6-oxohexyl)thioureido)-2-(6-hydroxy-3-oxo-3H-xanthen-9-yl)benzoic acid (MAT379, 24)

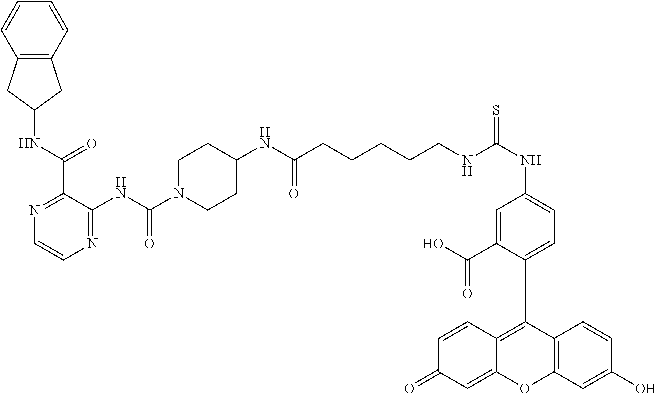 [SMILES: O=C(CCCCCNC(=S)Nc1ccc(-c2c3ccc(=O)cc-3oc3cc(O)ccc23)c(C(=O)O)c1)NC1CCN(C(=O)Nc2nccnc2C(=O)NC2Cc3ccccc3C2)CC1]

Combined 42·2HCl (21.6 mg, 40.7 mol, 1 eq), DMF (1 mL), and DI PEA (30 μL, 22 mg, 0.17 mmol, 4.2 eq), and fluorescein 5/6-isothiocyanate (20.0 mg, 51.4 μmol, 1.25 eq). Stirred for 20 h. Reaction mixture was directly purified by reverse phase flash column chromatography (water+0.1% formic acid/MeCN+0.1% formic acid). Yield: 12.3 mg, 34.2%. Orange solid. ¹H NMR (400 MHz, DMSO) δ 11.00 (s, 1H), 10.13 (s, 2H), 9.98 (s, 1H), 9.21 (d. J=7.7 Hz, 1H), 8.50 (d, J=2.7 Hz, 1H), 8.25 (s, 1H), 8.22 (d, J=2.6 Hz, 1H), 8.14 (s, 1H), 7.83 (d, J=7.8 Hz, 1H), 7.80-7.65 (m, 1H), 7.27-7.05 (m, 5H), 6.67 (d, J=2.6 Hz, 2H), 6.64-6.52 (m, 4H), 4.80-4.62 (m, 1H), 3.97 (d, J=13.3 Hz, 2H), 3.88-3.72 (m, 1H), 3.49 (s, 1H), 3.19 (dd, J=15.8, 7.6 Hz, 2H), 3.05 (dd, J=15.7, 6.9 Hz, 4H), 2.14-2.01 (m, 1H), 1.87-1.69 (m, 2H), 1.63-1.45 (m, 4H), 1.45-1.15 (m, 6H). ¹³C NMR (101 MHz, DMSO) δ 171.32, 168.57, 165.66, 163.08, 159.47, 152.19, 151.87, 149.97, 145.71, 141.43, 141.10, 135.50, 130.49, 129.06, 126.47, 124.46, 124.04, 112.58, 109.72, 102.23, 82.99, 50.44, 45.52, 43.74, 42.80, 38.44, 35.40, 31.53, 28.19, 26.14, 25.11. LC-MS (C₄₇H₄₆N₈O₈S): Calculated [M+H]⁻ m/z=883.32, [M−H]⁻ m/z=881.31. Observed [M+H]⁺ m/z=883.74, [M−H]⁻ m/z=881.61.

### 4-(3-(6-(((1-((3-((2,3-dihydro-1H-inden-2-yl)carbamoyl)pyrazin-2-yl)carbamoyl)piperidin-4-yl)methyl)amino)-6-oxohexyl)thioureido)-2-(6-hydroxy-3-oxo-3H-xanthen-9-yl)benzoic acid (MAT425)

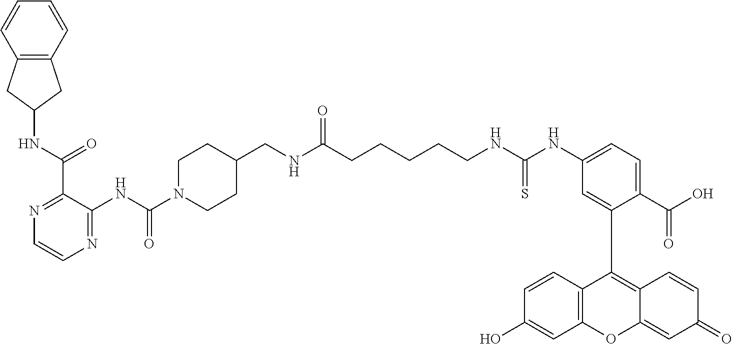 [SMILES: O=C(CCCCCNC(=S)Nc1ccc(C(=O)O)c(-c2c3ccc(=O)cc-3oc3cc(O)ccc23)c1)NCC1CCN(C(=O)Nc2nccnc2C(=O)NC2Cc3ccccc3C2)CC1]

Combined 44-2HCl (5.60 mg, 9.65 μmol, 1 eq), DIPEA (8.40 μL, 6.23 mg, 48.2 μmol, 5 eq), and DMSO (250 μL) and stirred one minute. Added solution of 5/6-FITC (3.76 mg, 9.65 μmol, 1 eq) in DMSO (250 μL) and stirred vigorously. Monitored by LCMS until reaction was complete. Purified reaction mixture directly by reverse phase flash column chromatography (water+0.1% formic acid/MeCN+0.1% formic acid). Yield: 3.2 mg, 37%. Yellow solid. ¹H NMR (400 MHz, DMSO) δ 11.00 (s, 1H), 10.23 (s, 2H), 9.20 (d, J=7.8 Hz, 1H), 8.49 (d, J=2.4 Hz, 1H), 8.41 (s, 1H), 8.31-8.17 (m, 2H), 7.86 (t, J=5.9 Hz, 1H), 7.75 (d, J=8.2 Hz, 1H), 7.27-7.18 (m, 2H), 7.18-7.11 (m, 2H), 6.68-6.52 (in. 5H), 4.72 (dt, J=15.1, 8.7 Hz, 1H), 4.05 (d, J=13.7 Hz, 2H), 3.51-3.42 (m, 2H), 3.19 (dd, J=15.7, 7.5 Hz, 2H), 3.11-3.00 (m, 2H), 2.97 (t, J=6.0 Hz, 1H), 2.92-2.76 (m, 2H), 2.25 (s, 1H), 2.14-2.04 (m, 2H), 1.68 (d, J=12.5 Hz, 2H), 1.61-1.48 (m, 4H), 1.40-1.04 (m, 12H), 0.84 (d, J=6.2 Hz, 11H). LC-MS (C₄₈M₄₈N₈O₈S): Calculated [M+H]⁺ m/z=897.34, [M−H]⁻ m/z=895.32. Observed [M+H]⁺ m/z=897.64, [M−H]− m/z=895.54.

The following compound were prepared in a manner similar to that used for preparation of compounds MAT379 and MAT425:

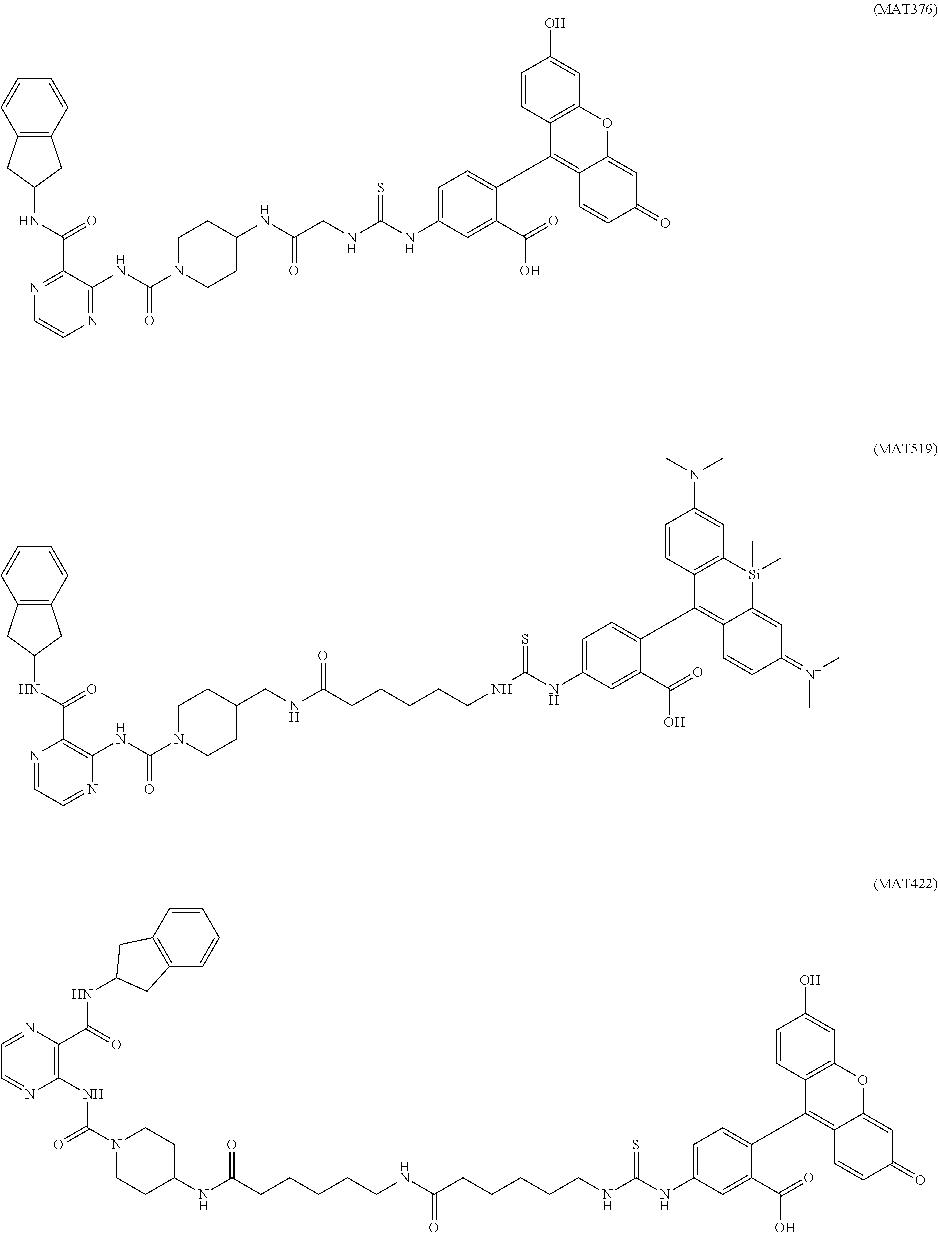 [SMILES: O=C(CNC(=S)Nc1ccc(-c2c3ccc(=O)cc-3oc3cc(O)ccc23)c(C(=O)O)c1)NC1CCN(C(=O)Nc2nccnc2C(=O)NC2Cc3ccccc3C2)CC1 ; CN(C)c1ccc2c(c1)[Si](C)(C)C1=CC(=[N+](C)C)C=CC1=C2c1ccc(NC(=S)NCCCCCC(=O)NCC2CCN(C(=O)Nc3nccnc3C(=O)NC3Cc4ccccc4C3)CC2)cc1C(=O)O ; O=C(CCCCCNC(=S)Nc1ccc(-c2c3ccc(=O)cc-3oc3cc(O)ccc23)c(C(=O)O)c1)NCCCCCC(=O)NC1CCN(C(=O)Nc2nccnc2C(=O)NC2Cc3ccccc3C2)CC1]

Discussion of FRET tracers MAT376, MAT519, and MAT422

The alternative tracers such as MAT422 (K_{D, PfcProRS}=1.28 μM and K_{D,HsProRS}>4 μM) had lower affinity compared to MAT379 and MAT425. The results show that thiourea contributed significantly to the selectivity for PfcProRS which demonstrated that selectivity is possible.

### 2-(((3aR,4R,6R,6aR)-6-(6-amino-9H-purin-9-yl)-2,2-dimethyltetrahydrofuro[3,4-d][1,3]dioxol-4-yl)methyl)isoindoline-1,3-dione (MAT520)

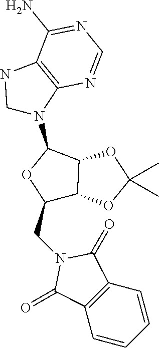 [SMILES: CC1(C)O[C@@H]2[C@H](O1)[C@@H](CN1C(=O)c3ccccc3C1=O)O[C@H]2N1CNc2c(N)ncnc21]

Synthesized according to a combination of the protocols reported previously with minor modifications. Charged flask with 2′,3′-O-isopropylideneadenosine (23.2 g, 75.4 mmol, 1 eq) and THF (200 mL). To the suspension, added triphenylphosphine (26.6 g, 101 mmol, 1.34 eq) and phthalimide (11.9 g, 80.9 mmol, 1.07eq). Stirred vigorously and flushed with argon for 10 minutes at room temperature. Kept under a positive pressure argon atmosphere for duration of the reaction. Over the course of 10 minutes, added diisopropyl azodicarboxylate (DIAD, 16.9 mL, 17.4 g, 85.8 mmol, 1.14 eq) slowly along the walls. Monitored reaction by LCMS and worked up when complete which was typically ˜24h (note: letting this reaction go to completion makes purification of the next step substantially easier). At the end of the reaction, had a large quantity of insoluble material that contained the product. Filtered and discarded the filtrate. Washed solid with Et₂O (800 mL total split over four washes), while doing so periodically stirred with spatula to ensure ether flowed through evenly and let it sit in the ether for at least two minutes before filtering. Dried solid in vacuo. Yield: 19.82 g, 60.26%. White solid. ¹H NMR (400 MHz, DMSO) δ 8.28 (s, 1H), 7.86 (s, 1H), 7.81 (s, 4H), 7.31 (s, 2H), 6.18 (d, J=2.0 Hz, 1H), 5.44 (dd, J=6.3, 2.0 Hz, 1H), 5.18 (dd, J=6.3, 3.7 Hz, 1H), 4.37 (td, J=6.0, 3.7 Hz, 1H), 3.99-3.80 (m, 2H), 1.50 (s, 3H), 1.30 (s, 3H). ¹³C NMR (101 MHz, DMSO) δ 167.76, 156.06, 152.47, 148.63, 140.33, 134.45, 131.43, 123.08, 119.24, 113.49, 88.70, 83.77, 83.43, 81.75, 67.02, 64.93, 27.01, 25.26, 25.13, 15.18. LC-MS (C₂₁H₂₀N₆O₅): Calculated [M+H]⁺ m/z=437.16, [M−H]⁻ m/z=435.14. Observed [M+H]⁺ m/z=437.34, [M−H]-m/z=435.34.

### 5′-Amino-5′-deoxy-2′,3′-O-isopropylidene-adenosine (MAT521)

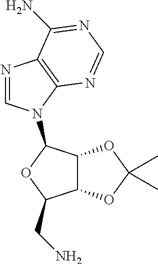 [SMILES: CC1(C)O[C@@H]2[C@H](O1)[C@@H](CN)O[C@H]2n1cnc2c(N)ncnc21]

Synthesized using route described by Kolb et al. but purified differently.^{15, 16} Charged flask with MAT520 (8.16 g, 18.7 mmol, 1 eq), ethanol (575 mL), and hydrazine monohydrate (14.7 mL, 15.0 g. 299 mmol, 16 eq). Refluxed overnight. Cooled to room temperature, filtered, and concentrated to a white solid (>9:1 mixture of MAT521 and 2′,3′-O-isopropylidene-adenosine). Optionally purified by flash column chromatography (DCM/MeOH) to obtain MAT521 as a white solid. Yield: 5.32 g, 93%. White solid. ¹H NMR (400 MHz, DMSO) δ 8.37 (s, 1H), 8.16 (s, 1H), 7.36 (s, 2H), 6.08 (d, J=3.2 Hz, 1H), 5.45 (dd, J=6.3, 3.2 Hz, 1H), 4.98 (dd, J=6.3, 2.7 Hz, 1H), 4.09 (td, J=5.8, 2.6 Hz, 11H), 2.77-2.63 (m, 2H), 1.53 (s, 3H), 1.32 (s, 3H). ¹H NMR (400 MHz, MeOD) δ 8.27 (s, 1H), 8.21 (s, 1H), 6.14 (d. J=3.0 Hz, 1H), 5.47 (dd, J=6.5, 3.0 Hz, 1H), 5.01 (dd, J=6.4, 3.4 Hz, 1H), 4.22 (td, J=5.7, 3.4 Hz, 1H), 2.92-2.85 (m, 2H), 1.59 (s, 3H), 1.38 (s, 3H). ¹³C NMR (101 MHz, DMSO) δ 156.16, 152.75, 149.01, 139.98, 119.18, 113.15, 89.14, 87.04, 82.71, 81.63, 43.72, 27.08, 25.25. LC-MS (C₁₃H₁₈N₆O₃): Calculated [M+H]⁺ m/z=307.15. Observed [M+H]⁺ m/z=370.30.

### 9-[(3aS,4R,6R)-2,2-dimethyl-6-[(sulfamoylamino)methyl]-3a,4,6,6a-tetrahydrofuro[3,4-d]1,3]dioxol-4-yl]-6-aminopurine (MA1495)

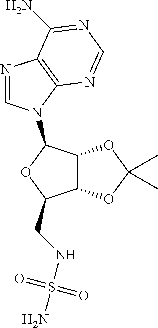 [SMILES: CC1(C)O[C@@H]2[C@H](O1)[C@@H](CNS(N)(=O)=O)O[C@H]2n1cnc2c(N)ncnc21]

This compound has been previously reported in a patent with minimal S procedural information and zero characterization data. Synthesized using the general sulfamide coupling protocol developed by Meng et al. for the synthesis of structurally unrelated compounds. In short, charged vial with MAT521 (112 mg, 366 μmol, 1 eq), sulfuric diamide (93.9 mg, 977 μmol, 2.67 eq), and water (1 mL). Stirred vigorously and refluxed for 2.75 h. Purified by reverse-phase flash column chromatography (water/MeCN, both with 0.1% formic acid). Yield: 131 mg, 93%. White solid. ¹H NMR (400 MHz, CDCl₃) δ 8.33 (s, 1H), 8.16 (s, 1H), 7.42 (s, 2H), 7.38-7.27 (m, 1H), 6.63 (s, 2H), 6.10 (d, J=3.4 Hz, 1H), 5.38 (dd, J=6.3, 3.5 Hz, 1H), 5.01 (dd, J=6.3, 2.5 Hz, 1H), 4.34 (td, J=5.4, 2.5 Hz, 1H), 3.25-3.04 (m, 2H), 1.55 (s, 3H), 1.32 (s, 3H). ¹³C NMR (101 MHz, CDCl₃) δ 156.30, 152.78, 152.66, 148.53, 140.21, 140.09, 119.41, 113.42, 89.93, 83.73, 82.53, 81.70, 48.62, 44.46, 27.09, 25.24. LC-MS (C₁₃H₁₉N₇O₅S): Calculated [M+H]⁺ m/z=386.12, [M−H]⁻ m/z=384.11. Observed [M+H]⁺ m/z=386.29, [M−H]⁻ m/z=384.25.

Note: purification fails to remove the MAT16 byproduct if the MAT521 starting material is contaminated with 2′,3′-O-isopropylidene-adenosine.

### tert-butyl (S)-2-((N-(((3aR,4R,6R,6aR)-6-(6-amino-9H-purin-9-yl)-2,2-dimethyltetrahydrofuro[3,4-d][1,3]dioxol-4-yl)methyl)sulfamoyl)carbamoyl)pyrrolidine-1-carboxylate (MAT498)

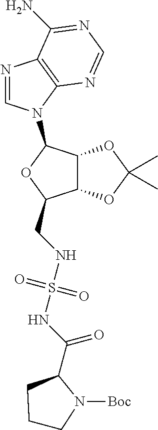 [SMILES: CC(C)(C)OC(=O)N1CCC[C@H]1C(=O)NS(=O)(=O)NC[C@H]1O[C@@H](n2cnc3c(N)ncnc32)[C@@H]2OC(C)(C)O[C@@H]21]

Charged vial with MAT495 (25.0 mg, 64.9 μmol, 1 eq), BOC-L-proline N-hydroxysuccinimide ester (24.3 mg, 77.8 μmol, 1.2 eq), DMF (500 μL), and DBU (23.5 μL, 23.7 mg, 156 μmol, 2.4 eq). After reaction was complete by LCMS (˜15 minutes), purified reaction mixture directly by reverse-phase flash column chromatography (water/MeCN). LC-MS (C₂₃H₃₄N₈O₈S): Calculated [M+H]⁺ m/z=583.23, [M−H]⁻ m/z=581.21. Observed [M+H]⁺ m/z=583.38, [M−H]⁻ m/z=581.40.

### (S)-N-(N-(((2R,3S,4R,5R)-5-(6-amino-9H-purin-9-yl)-3,4-dihydroxytetrahydrofuran-2-yl)methyl)sulfamoyl)pyrrolidine-2-carboxamide (ProSNA: MAT499)

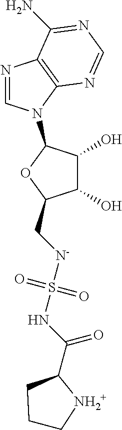 [SMILES: Nc1ncnc2c1ncn2[C@@H]1O[C@H](C[N-]S(=O)(=O)NC(=O)[C@@H]2CCC[NH2+]2)[C@@H](O)[C@H]1O]

Charged vial with MAT498 (58.0 mg, 99.5 μmol, 1 eq) and 5:2 TFA/water (2.1 mL). Stirred vigorously for 40 minutes. Concentrated in vacuo and then azeotroped 3× with methanol (2 mL). Purified by reverse-phase flash column chromatography (water/MeCN) to obtain ProSNA as the mono-trifluoroacetic acid salt. Yield: Quantitative. White solid. ¹H NMR (400 MHz, DMSO) δ 9.64-9.22 (m, 2H), 8.70 (s, 1H), 8.40 (s, 1H), 8.31 (s, 1H), 7.97 (s, 2H), 5.86 (d, J=6.7 Hz, 1H), 4.71 (dd, J=6.8, 5.0 Hz, 1H), 4.21 (d, J=9.2 Hz, 1H), 4.14 (q, J=3.7 Hz, 1H), 4.10 (dd, J=5.0, 2.3 Hz, 1H), 3.96 (s, 1H), 3.31-3.12 (m, 4H), 2.40-2.28 (m, 1H), 1.93-1.78 (m, 3H). ¹³C NMR (101 MHz, DMSO) δ 167.65, 158.46, 148.24, 141.42, 119.59, 88.78, 83.53, 72.44, 71.33, 59.46, 45.78, 29.25, 23.35. Note: ¹³C NMR spectra had insufficient signal to background to see all expected resonances but listed is what was observed. including the peaks at 119.59 and 158.46 which are likely from the trifluoroacetic acid. LC-MS (C₁₅H₂₂N₈O₆S): Calculated [M+H]⁺ m/z=443.15, [M−H]⁻ m/z=441.13. Observed [M+H]⁺ m/z=443.25, [M−H]⁻ m/z=441.34.

### tert-butyl ((2S,3S)-1-((N-(((3aR,4R,6R,6aR)-6-(6-amino-9H-purin-9-yl)-2,2-dimethyltetrahydrofuro[3,4-d][1,3]dioxol-4-yl)methyl)sulfamoyl)amino)-3-methyl-1-oxopentan-2-yl)carbamate (MAT529)

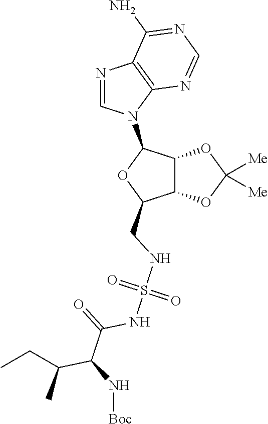 [SMILES: CC[C@H](C)[C@H](NC(=O)OC(C)(C)C)C(=O)NS(=O)(=O)NC[C@H]1O[C@@H](n2cnc3c(N)ncnc32)[C@@H]2OC(C)(C)O[C@@H]21]

Charged vial with MAT495 (199 mg, 516 μmol, 1 eq), BOC-L-isoleucine N-hydroxysuccinimide ester (207 mg, 631 μmol, 1.2 eq), DMF (4 mL), and DBU (188 μL, 190 mg, 1.25 mmol, 2.4 eq). After stirring for 1.5 h, added more BOC-L-isoleucine N-hydroxysuccinimide ester (113 mg). Concentrated to ˜0.5 mL of a viscous oil. Purified by reverse-phase flash column chromatography (water/MeCN, both with 0.1% formic acid) to obtain MAT529 contaminated with 0.4 molar equivalents of BOC-L-isoleucine. Typically deprotected with the impurity and subsequently purified, but for chemical characterization, was further purified by flash column chromatography (EtOAc/EtOH). Yield: 215 mg, 70% yield (not corrected for impurity). White solid. If further purified, 63% yield. ¹H NMR (400 MHz, DMSO) δ 11.46 (s, 1H), 8.59 (s, 1H), 8.30 (s, 1H), 8.17 (s, 1H), 7.41 (s, 2H), 6.94 (d, J=8.4 Hz, 1H), 6.11 (d, J=3.2 Hz, 1H), 5.36 (dd, J=6.2, 3.2 Hz, 1H), 4.97 (dd, J=6.3, 2.5 Hz, 1H), 4.35-4.27 (m, 1H), 3.81 (t, J=8.1 Hz, 1H), 3.20 (dt, J=11.6, 5.5 Hz, 1H), 3.10 (dt, J=13.0, 5.9 Hz, 1H), 1.69-1.59 (m, 1H), 1.54 (s, 3H), 1.42-1.20 (m, 14H), 1.15-1.01 (m, 1H), 0.81-0.70 (m, 6H). ¹³C NMR (101 MHz, DMSO) δ 156.29, 155.45, 152.77, 148.37, 140.12, 119.41, 113.40, 89.96, 83.70, 82.68, 81.77, 78.27, 58.91, 44.62, 35.80, 28.14, 27.06, 25.21, 24.30, 21.09, 15.16, 10.70. LC-MS (C₂₄H₃₈N₈O₈S): Calculated [M+H]⁺ m/z=599.26, [M−H]⁻ m/z=597.25. Observed [M+H]⁺ m/z=599.92, [M−H]⁻ m/z=597.38.

### (2S,3S)-2-amino-N-(N-(((2R,3S,4R,5R)-5-(6-amino-9H-purin-9-yl))-3,4-dihydroxytetrahydrofuran-2-yl)methyl)sulfoaroyl)-3-methylpentanamide (IleSNA: MAT530)

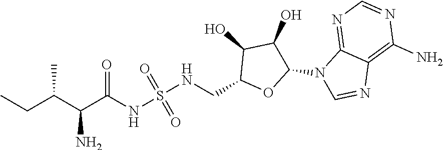 [SMILES: CC[C@H](C)[C@H](N)C(=O)NS(=O)(=O)NC[C@H]1O[C@@H](n2cnc3c(N)ncnc32)[C@H](O)[C@@H]1O]

Charged vial with impure MAT529 (99.5 mg, 166 μmol, 1 eq: impurity is 0.4 molar equivalents of BOC-L-isoleucine) and 5:2 TFA/water (1.4 mL). Stirred vigorously for 50 minutes. Concentrated in vacuo and then azeotroped with 3× methanol (2 mL). Purified by reverse-phase flash column chromatography (water/MeCN) to obtain IleSNA as a white solid. ¹H NMR (400 MHz, DMSO) δ 8.29 (s, 1H), 8.20 (s, 1H), 7.90 (s, 2H), 7.35 (s, 2H), 5.82 (d, J=6.6 Hz, 1H), 5.46 (d, J=6.2 Hz, 1H), 5.27 (s, 1H), 4.73 (q, J=5.5 Hz, 1H), 4.17-4.07 (m, 2H), 4.04 (q, J=4.0 Hz, 1H), 3.40 (s, 1H), 3.07 (t, J=4.6 Hz, 2H), 1.81 (dp, J=15.7, 5.6 Hz, 1H), 1.52-1.38 (m, 1H), 1.21-1.03 (m, 1H), 0.89 (d, J=7.0 Hz, 3H), 0.82 (t, J=7.3 Hz, 3H). ¹³C NMR (101 MHz, DMSO) δ 156.21, 152.66, 148.95, 140.42, 119.49, 88.21, 83.79, 72.40, 71.33, 48.62, 45.22, 36.36, 24.19, 14.91, 11.66. LC-MS (C₁₆H₂₆N₈O₆S): Calculated [M+H]⁺ m/z=459.18, [M−H]⁻ m/z=457.16. Observed [M+H]⁺ m/z=459.26, [M−H]⁻ m/z=457.41.

### tert-butyl ((S)-1-((N-(((3aR,4R,6R,6aR)-6-(6-amino-9H-purin-9-yl)-2,2-dimethyltetrahydrofuro[3,4-d][1,3]dioxol-4-yl)methy)sulfamoyl)amino)-1-oxo-3-phenylpropan-2-yl)carbamate (DPG17)

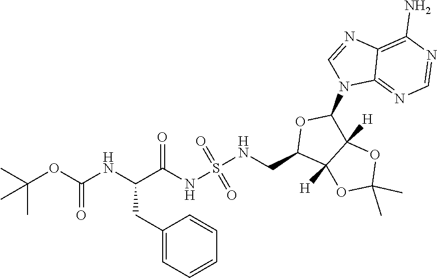 [SMILES: CC(C)(C)OC(=O)N[C@@H](Cc1ccccc1)C(=O)NS(=O)(=O)NC[C@H]1O[C@@H](n2cnc3c(N)ncnc32)[C@@H]2OC(C)(C)O[C@@H]21]

Charged vial with MAT495 (152 mg, 393 μmol, 1 eq), BOC-L-phenylalanine N-hydroxysuccinimide ester (173 mg, 478 μmol, 1.2 eq), DMF (3.5 mL), and DBU (142 μL, 144 mg, 0.944 mmol, 2.4 eq). Stirred for 1.25 h. Concentrated to ˜0.5 mL of a viscous oil. Purified by reverse-phase flash column chromatography (water/MeCN, both with 0.1% formic acid) to obtain DPG17 contaminated with sub-stoichiometric BOC-L-phenylalanine. Used without further purification. Yield: 220 mg, 88% (not corrected for impurity). White solid. ¹H NMR (400 MHz, DMSO) δ 11.71 (s, 1H), 8.70 (t, J=6.0 Hz, 1H), 8.32 (s, 1H), 8.21 (s, 1H), 7.45 (s, 2H), 7.19 (ddt, J=47.5, 16.9, 6.8 Hz, 8H), 6.12 (d, J=3.5 Hz, 1H), 5.34 (dd, J=6.2, 3.3 Hz, 1H), 4.96 (dd, J=6.2, 2.6 Hz, 1H), 4.36-4.29 (m, 1H), 4.17 (ddd, J=11.9, 8.3, 4.0 Hz, 1H), 3.12 (q, J=7.3 Hz, 2H), 3.05-2.76 (m, 2H), 2.70 (dd, J=13.6, 10.8 Hz, 1H), 1.55 (s, 3H), 1.38-1.14 (m, 16H). ¹³C NMR (101 MHz, DMSO) δ 173.64, 171.18, 163.07, 156.18, 155.47, 155.37, 152.68, 148.35, 140.19, 138.06, 137.52, 129.26, 129.10, 128.15, 128.05, 126.38, 126.32, 119.43, 113.45, 90.06, 83.44, 82.65, 81.70, 78.28, 78.04, 56.09, 55.17, 44.67, 40.43, 36.63, 36.43, 28.16, 28.10, 28.00, 27.66, 27.09, 25.23. LC-MS (C₂H₃₆N₈O₈S): Calculated [M+H]⁻ m/z=633.24, [M−H]⁻ m/z=631.23. Observed [M+H]⁺ m/z=633.00, [M−H]⁻ m/z=631.43.

### (S)-amino-N-(N-(((2R,3S,4R,5R)-5-(6-amino-9H-purin-9-yl)-3,4-dihydroxytetrahydrofuran-2-y)methyl)sulfamoyl)-3-phenylpropanamide (PheNSA: DPG21)

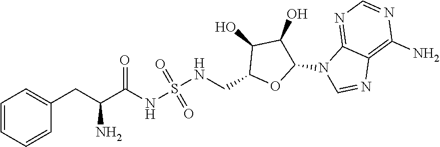 [SMILES: Nc1ncnc2c1ncn2[C@@H]1O[C@H](CNS(=O)(=O)NC(=O)[C@@H](N)Cc2ccccc2)[C@@H](O)[C@H]1O]

Charged vial with impure DPG17 (262 mg, 414 μmol, 1 eq: impurity is sub-stoichiometric BOC-L-phenylalanine) and 5:2 TFA/water (2 mL). Stirred vigorously for 35 minutes. Concentrated in vacuo and then azeotroped with 5× methanol (˜2 mL). Purified by reverse-phase flash column chromatography (water/MeCN) to obtain PheSNA. Yield: 135 mg, 66%. White solid. ¹H NMR (400 MHz, DMSO) δ 8.30 (s, 1H), 8.23 (s, 1H), 7.38 (s, 2H), 7.27 (dq, J=11.7, 6.3 Hz, 5H), 5.82 (d, J=6.8 Hz, 1H), 5.49 (s, 1H), 5.30 (s, 1H), 4.75 (s, 1H), 4.14-4.03 (m, 2H), 3.83 (s, 1H), 3.17 (s, 1H), 3.12 (d, J=4.9 Hz, 1H), 3.07-2.86 (m, 3H). ¹³C NMR (101 MHz, DMSO) δ 156.31, 153.04, 149.01, 141.07, 129.86, 128.95, 127.53, 119.70, 88.81, 84.08, 72.56, 71.57, 55.10, 55.08, 48.85, 45.31, 37.13. LC-MS (C₁₉H₂₄N₈O₆S): Calculated [M+H]⁺ m/z=493.16, [M−H]⁻ m/z=491.15. Observed [M+H]⁺ m/z=493.55, [M−H]⁻ m/z=491.41. After adding D20: ¹H NMR (400 MHz, DMSO) δ 8.28 (s, 1H), 8.22 (s, 1H), 7.26 (dt, J=13.7, 7.6 Hz, 5H), 5.80 (d, J=6.9 Hz, 1H), 4.75-4.70 (m, 1H), 4.13-4.03 (m, 2H), 3.83 (s, 1H), 3.15 (s, 1H), 3.13-2.88 (m, 4H).

### tert-butyl (2-(2-((6-iodohexyl)oxoethoxy)ethyl)carbamate (MAT373)

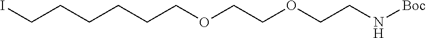 [SMILES: CC(C)(C)OC(=O)NCCOCCOCCCCCCI]

Combined tert-butyl (2-(2-((6-chlorohexyl)oxy)ethoxy)ethyl)carbamate (908 mg, 2.81 mmol, 1 eq), acetone (10 mL), and sodium iodide (2.10 g, 14.0 mmol, 5 eq). Refluxed for 18 h. Diluted with water (100 mL), extracted 3× with DCM (100 mL each), dried over Na₂SO₄, filtered, and concentrated in vacuo to obtain MAT373. Yield: 1.13 g, 97%. White solid (some samples have light orange hue)., ¹H NMR (400 MHz, CDCl₃) δ 5.00 (s, 1H), 3.63-3.49 (m, 6H), 3.45 (t, J=6.6 Hz, 2H), 3.30 (q, J=4.7 Hz, 2H), 3.17 (t, J=7.0 Hz, 2H), 1.81 (p, J=6.9 Hz, 2H), 1.59 (p, J=6.9 Hz, 2H), 1.45-1.32 (m, 13H). ¹³C NMR (101 MHz, CDCl₃) δ 156.09, 79.30, 71.38, 70.38, 70.32, 70.14, 40.47, 33.51, 30.39, 29.49, 28.54, 25.17, 7.23, LC-MS (C₁₅H₃₀INO₄): Calculated [M+H]⁺ m/z=416.13, [M−BOC+2H]⁺ m/z=316.08, [M+Na]⁺ m/z=438.11. Observed [M+H]⁺ m/z=416.26, [M−BOC+2H]⁺ m/z=316.19, [M+Na]⁺ m/z=438.22.

### tert-butyl (2-(2-((6-((((3aR,4R,6R,6aR)-6-(6-amino-9H-purin-9-yl)-2,2-dimethyltetrahydrofuro[3,4-d][1,3]dioxol-4-yl)methyl)amino)hexyl)oxy)ethoxy)ethyl)arbamate (MAT500)

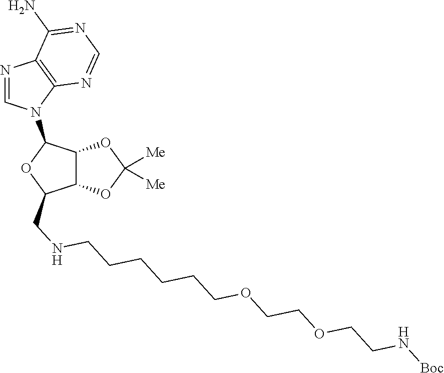 [SMILES: CC(C)(C)OC(=O)NCCOCCOCCCCCCNC[C@H]1O[C@@H](n2cnc3c(N)ncnc32)[C@@H]2OC(C)(C)O[C@@H]21]

Charged flask with MAT373 (496 mg, 1.19 mmol, 1 eq), MAT521 (1.10 g, 3.58 mmol, 3 eq), MeCN (25 mL), and DIPEA (1.04 mL, 771 mg, 5.97 mmol, 5 eq). Heated to 70° C. for 48 h. Concentrated to an oil. Purified by reverse phase flash column chromatography (water/MeCN, both with 0.1% formic acid) and then by flash column chromatography (DCM/MeOH). Yield: 448 mg, 63.3%. Off-white solid. Also, separately recovered much of the excess MAT521, ¹H NMR (400 MHz, DMSO) δ 8.35 (s, 1H), 8.15 (s, 1H), 7.34 (s, 2H), 6.75 (t, J=5.8 Hz, 1H), 6.10 (d, J=2.9 Hz, 1H), 5.47 (dd, J=6.3, 2.9 Hz, 1H), 4.96 (dd, J=6.3, 2.8 Hz, 1H), 4.21 (td, J=6.1, 2.6 Hz, 1H), 3.52-3.41 (m, 5H), 3.41-3.28 (m, 10H), 3.05 (q, J=6.0 Hz, 2H), 2.71 (qd, J=12.4, 6.0 Hz, 2H), 2.45 (t, J=7.0 Hz, 2H), 1.53 (s, 3H), 1.44 (p, J=6.7 Hz, 2H), 1.34 (d, J=15.9 Hz, 15H), 1.26-1.17 (m, 4H). Note: the peak at 3.34 overlaps with the water peak so the actual number of resonances could not be determined. We have reported the count for the combined water+molecule peak. ¹³C NMR (101 MHz, DMSO) δ 156.16, 155.58, 152.68, 148.92, 140.00, 119.23, 113.17, 89.28, 84.86, 82.73, 82.19, 77.57, 70.29, 69.50, 69.42, 69.15, 50.90, 49.15, 29.19, 29.09, 28.23, 27.04, 26.52, 25.57, 25.23, LC-MS (C₂₈H₄₇N₇O₇): Calculated [M+H]⁺ m/z=594.36, [M−H]⁻ m/z=592.35. Observed [M+H]⁺ m/z=593.96, [M−H]⁻ m/z=592.41.

### tert-butyl (S)-2-((N-(((3aR,4R,6R,6aR)-6-(6-amino-9H-purin-9-yl)-2,2-dimethyltetrahydrofuro[3,4-d][1,3]dioxol-4-yl)methyl)-N-(2,2-dimethyl-4-oxo-3,8,11-trioxa-5-azaheptadecan-17-yl)sulfamoyl)carbamoyl)pyrrolidine-1-carboxylate (MAT555)

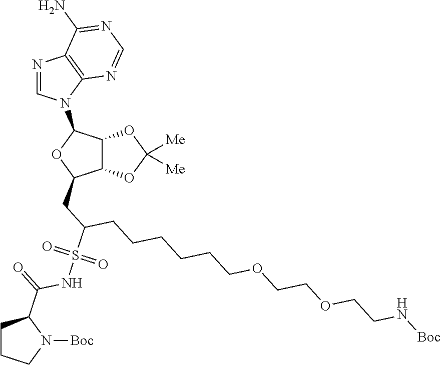 [SMILES: CC(C)(C)OC(=O)NCCOCCOCCCCCCC(C[C@H]1O[C@@H](n2cnc3c(N)ncnc32)[C@@H]2OC(C)(C)O[C@@H]21)S(=O)(=O)NC(=O)[C@@H]1CCCN1C(=O)OC(C)(C)C]

Charged vial with MAT500 (250 mg. 421 μmol, 1 eq) and MeCN (3 mL). Stirred vigorously and flushed with argon (kept under positive pressure of argon for duration of reaction). Added DBU (635 μL, 640 mg, 4.21 mmol, 10 eq). Charged second vial with MAT15 (360 mg. 3.12 mmol, 7.4 eq) and MeCN (5 mL). Using a syringe pump, slowly added the MAT15 solution to the MAT500 over the course of 1 h. Stirred 15 minutes after the addition was complete to obtain tert-butyl (2-(2-((6-((((3aR,4R,6R,6aR)-6-(6-amino-9H-purin-9-yl)-2,2-dimethyltetrahydrofuro[3,4-d][1,3]dioxol-4-yl)methyl)(sulfamoyl)amino)hexyl)oxy)ethoxy)ethyl)carbamate (MAT501) which was used without purification., To this mixture, added DBU (525 μL, 530 mg, 3.48 mmol, 8.27 eq) and stirred vigorously for 2 minutes. Over the course of ˜15 seconds, slowly added premixed solution of BOC-L-proline N-hydroxysuccinimide ester (449 mg, 1.44 mmol, 3.41 eq) in MeCN (3 mL). After 1.25 h, diluted with DCM (200 mL) and MeOH (10 mL). Washed with a mixture of water (200 mL) and brine (50 mL). Back extracted from the water/brine mixture with a mixture of DCM (200 mL) and MeOH (20 mL). Dried over Na₂SO₄, filtered, and concentrated. Purified by flash column chromatography (EtOAc->1:1:1 water/MeOH/MeCN) and then by reverse phase flash column chromatography (water/MeCN). Yield: 38%. White solid, ¹H NMR (400 MHz, DMSO) δ 11.69 (d, J=14.7 Hz, 1H), 8.38 (s, 1H), 8.21 (s, 1H), 7.73-7.48 (m, 2H), 6.77 (t, J=5.8 Hz, 1H), 6.24 (dd, J=6.7, 2.0 Hz, 1H), 5.48 (td, J=5.8, 2.0 Hz, 1H), 5.04 (ddd, J=10.3, 6.2, 3.0 Hz, 1H), 4.37 (tt, J=6.2, 3.6 Hz, 11H), 4.09 (ddd, J=11.9, 8.3, 3.4 Hz, 1H), 3.82 (ddd, J=25.7, 15.3, 4.5 Hz, 1H), 3.52-3.30 (m, 10H), 3.27 (t, J=6.6 Hz, 3H), 3.07 (q, J=12.0 Hz, 2H), 3.04-2.95 (m, 1H), 2.84 (dddd, J=20.4, 14.8, 9.8, 6.1 Hz, 1H), 2.19-2.02 (m, 1H), 1.84-1.68 (m, 3H), 1.54 (s, 3H), 1.38 (d, J=4.6 Hz, 12H), 1.32 (d, J=6.9 Hz, 12H), 1.19-1.08 (m, 2H), 1.06-0.94 (m, 2H), 0.92-0.76 (m, 2H). ¹³C NMR (101 MHz, DMSO) δ 171.58, 171.25, 155.59, 153.41, 152.85, 151.95, 148.42, 148.37, 140.71, 119.25, 113.35, 113.28, 89.41, 89.26, 86.22, 85.87, 83.22, 82.17, 82.09, 78.92, 78.90, 77.59, 70.20, 70.17, 69.51, 69.38, 69.17, 59.20, 59.14, 50.60, 49.86, 49.77, 46.50, 46.33, 40.43, 30.50, 29.58, 29.04, 29.00, 28.23, 28.10, 27.83, 26.97, 26.92, 26.89, 25.79, 25.71, 25.16, 23.83, 23.05, LC-MS (C₃₈H₆₃N₉O₁₂S): Calculated [M+H]⁻ m/z=870.44, [M−H]⁻ m/z=868.42. Observed [M+H]⁺ m/z=870.33, [M−H]⁻ m/z=868.78.

### (S)-N-(N-(((2R,3S,4R,5R)-5-(6-amino-9H-purin-9-yl)-3,4-dihydroxytetrahydrofuran-2-yl)methyl)-N-(6-(2-(2-aminoethoxy)ethoxy)hexyl)sulfamoyl)pyrrolidine-2-carboxamide (MAT556)

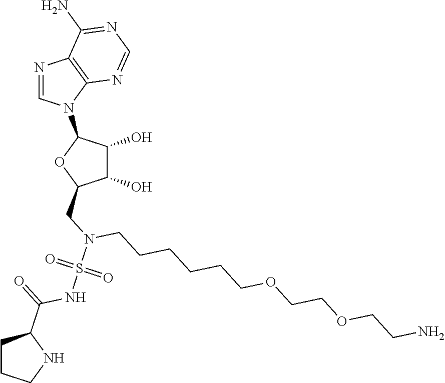 [SMILES: NCCOCCOCCCCCCN(C[C@H]1O[C@@H](n2cnc3c(N)ncnc32)[C@H](O)[C@@H]1O)S(=O)(=O)NC(=O)[C@@H]1CCCN1]

Charged vial with MAT555 (107 mg, 122 μmol, 1 eq) and 5:1 TFA/water (1.2 mL). Stirred vigorously for 30 min, concentrated in vacuo, and azeotroped 3× with MeOH (2 mL). Purified by reverse phase flash column chromatography (water/MeCN)., Yield: 39.9 mg, 51.8%. White solid., ¹H NMR (400 MHz, DMSO) δ 8.46-7.64 (m, 6H), 7.28 (s, 2H), 5.84 (d, J=6.3 Hz, 1H), 5.43 (s, 1H), 4.79 (t, J=5.7 Hz, 1H), 4.13 (dd, J=4.9, 3.0 Hz, 1H), 4.08 (td, J=6.2, 2.9 Hz, 11H), 3.82 (dd, J=8.5, 6.4 Hz, 1H), 3.66 (dd, J=15.0, 5.5 Hz, 1H), 3.61-3.53 (m, 4H), 3.47 (dd, J=5.9, 3.5 Hz, 2H), 3.27 (t, J=6.7 Hz, 2H), 3.24-3.00 (m, 5H), 2.97 (t, J=5.3 Hz, 2H), 2.90 (dq, J=14.5, 6.9 Hz, 1H), 2.54 (s, 1H), 2.13 (dq, J=12.6, 7.4 Hz, 1H), 1.97-1.70 (m, 3H), 1.54 (dd, J=13.4, 6.8 Hz, 1H), 1.41-1.28 (m, 4H), 1.16-1.02 (m, 4H). ¹³C NMR (101 MHz, DMSO) δ 177.01, 170.56, 156.23, 156.07, 152.61, 149.53, 149.04, 140.48, 140.10, 88.62, 87.31, 84.18, 79.93, 72.51, 72.23, 71.70, 71.61, 70.42, 70.25, 70.20, 69.78, 69.72, 69.32, 66.73, 61.66, 50.22, 48.83, 48.62, 47.18, 45.43, 40.43, 38.68, 38.61, 29.43, 29.06, 28.96, 28.02, 26.16, 25.78, 25.34, 25.21, 25.16, 24.77, 23.53, LC-MS (C₂₁H₄₃N₉O₈S): Calculated [M+H]⁻ m/z=630.30, [M−H]⁻ m/z=628.29. Observed [M+H]⁺ m/z=630.53, [M−H]⁻ m/z=628.46.

### benzyl (2-(2-hydroxyethoxy)ethyl)carbamate (MAT560)

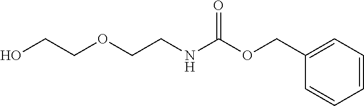 [SMILES: O=C(NCCOCCO)OCc1ccccc1]

Synthesized according to previous report.

### benzyl (2-(2-((6-chlorohexyl)oxy)ethoxy)ethyl)carbamate (MAT561)

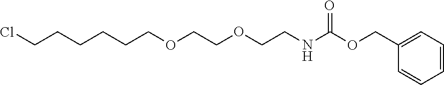 [SMILES: O=C(NCCOCCOCCCCCCCl)OCc1ccccc1]

Synthesized according to previous report.

### benzyl (2-(2-((6-iodohexyl)oxy)ethoxy)ethyl)carbamate (MAT562)

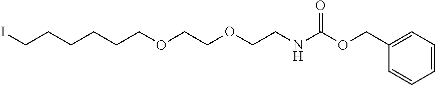 [SMILES: O=C(NCCOCCOCCCCCCI)OCc1ccccc1]

Combined benzyl (2-(2-((6-chlorohexyl)oxy)ethoxy)ethyl)carbamate (5.11 g, 14.3 mmol, 1 eq), acetone (50 mL), and sodium iodide (17.1 g, 114 mmol, 8 eq). Refluxed for 16 h. Diluted brine (200 mL), water (50 mL), and methanol (5 mL). Extracted 3× with EtOAc (200 mL), pooled EtOAc fractions, washed 3× with 1:1 brine/water (100 mL each wash), dried over Na₂SO₄, filtered, and concentrated in vacuo to obtain MAT562. Yield: 6.24 g, 97.4%. White solid (some samples have light orange hue). ¹H NMR (400 MHz, CDCl₃) δ 7.39-7.27 (m, 5H), 5.30 (s, 1H), 5.10 (s, 2H), 3.65-3.48 (m, 6H), 3.41 (dt, J=18.1, 5.9 Hz, 4H), 3.16 (t, J=7.0 Hz, 2H), 1.79 (p, J=6.9 Hz, 2H), 1.58 (p. J=6.8 Hz, 2H), 1.36 (dp, J=11.9, 6.9 Hz, 4H). ¹³C NMR (101 MHz, CDCl₃) δ 156.52, 136.68, 128.59, 128.16, 71.37, 70.42, 70.11, 70.10, 66.76, 41.00, 33.47, 30.34, 29.45, 25.14, 7.30. LC-MS (C₁₈H₂₈INO₄): Calculated [M+Na]⁺ m/z=472.10, [M+HCO₂]⁻ m/z=494.10. Observed [M+Na]⁺ m/z=472.31, [M−H]⁻ m/z=494.32.

### benzyl (2-(2-((6-((((3aR 4R 6R,6aR)-6-(6-amino-9H-purin-9-yl)-2,2-dimethyltetrahydrofuro[3,4-d][1,3]dioxol-4-yl)methyl)amino)hexyl)oxy)ethoxy)ethyl)arbamate (MAT563)

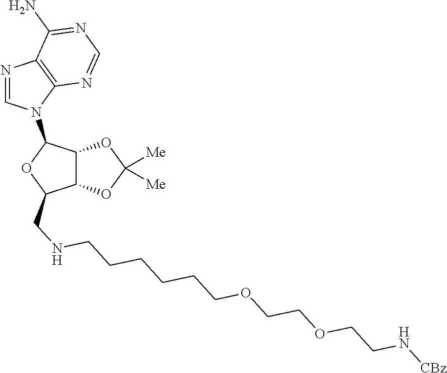

Charged flask with MAT562 (812 mg, 1.81 mmol, 1 eq), MAT521 (1.78 g, 5.79 mmol, 3.2 eq), MeCN (100 mL) and DIPEA (2 mL). Stirred vigorously and heated to 70° C. for 22 h. Added ˜8 g silica gel and concentrated in vacuo. Purified by flash column chromatography (dry load; DCM/MeOH) to obtain clean MAT563 and clean MAT521. Yield: 902 mg, 79.5% by isolated product (97% yield by recovered starting material). White solid. Also recovered 1.28 g MAT521 (72% recovery). ¹H NMR (400 MHz, DMSO) δ 8.37 (s, 1H), 8.17 (s, 1H), 7.42-7.23 (m, 8H), 6.20 (d, J=2.6 Hz, 1H), 5.46 (dd, J=6.2, 2.7 Hz, 1H), 5.07-5.03 (m, 1H), 5.02 (d, J=5.6 Hz, 2H), 4.32 (qd, J=4.9, 3.3 Hz, 1H), 3.52-3.43 (m, 4H), 3.41 (t, J=6.0 Hz, 2H), 3.34 (t, J=6.5 Hz, 2H), 3.15 (q, J=5.9 Hz, 2H), 3.07-2.93 (m, 2H), 2.61 (t, J=7.4 Hz, 2H), 1.55 (s, 3H), 1.48-1.35 (m, 4H), 1.33 (s, 3H), 1.21 (dt, J=16.6, 5.7 Hz, 6H). ¹³C NMR (101 MHz, DMSO) δ 156.20, 156.17, 152.71, 148.76, 140.16, 137.19, 128.35, 127.77, 127.72, 127.39, 119.27, 113.42, 89.27, 83.63, 82.94, 82.02, 70.22, 69.56, 69.41, 69.10, 65.22, 49.80, 48.25, 40.18, 29.06, 27.37, 27.01, 26.14, 25.37, 25.22.

LC-MS (C₃₁H₄₅N₇O₇): Calculated [M+H]⁺ m/z=628.35, [M−H]⁻ m/z=626.33. Observed [M+H]⁺ m/z=627.94, [M−H]⁻ m/z=626.51

### benzyl (2-(2-((6-((((3aR 4R,6R 6aR)-6-(6-amino-9H-purin-9-yl)-2,2-dimethyltetrahydrofuro[3,4-d][1,3]dioxol-4-yl)methyl)(sulfamoyl)amino)hexyl)oxy)ethoxy)ethyl)carbamate (MAT564)

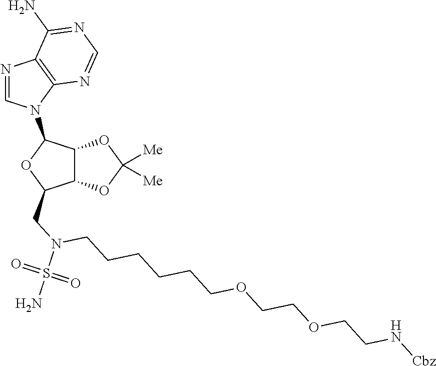 [SMILES: CC1(C)O[C@@H]2[C@H](O1)[C@@H](CN(CCCCCCOCCOCCNC(=O)OCc1ccccc1)S(N)(=O)=O)O[C@H]2n1cnc2c(N)ncnc21]

Charged flame dried flask with MAT563 (1.59 g, 2.54 mmol, 1 eq) and anhydrous MeCN. Stirred vigorously and cooled to 0° C. while flushing with argon for 30 min (kept under positive pressure of argon for duration of reaction). During this time, charged second flame dried flask with MAT15 (734 mg. 6.35 mmol, 2.5 eq) and MeCN (30 mL). Cooled second flask to cooled to 0° C. while flushing with argon. Slowly added the contents of the MAT15 solution along the walls of the MAT563 flask. Stirred vigorously at OC for 1.5 h. Over the course of ˜5 minutes, slowly added another 0° C. solution of MAT15 (1.09 g, 9.45 mmol, 3.73 eq) and MeCN (15 mL) that had been flushed with argon. Stirred at 0° C. for 0.5 h. Optionally purified by quenching with MeOH (0.5 mL), concentrating in vacuo to a solid, and purifying by reverse phase flash column chromatography (water/MeCN, both with 0.1% formic acid). Yield: 1.09 g, 61.1%. White solid. ¹H NMR (400 MHz, DMSO) δ 8.42 (s, 1H), 8.25 (s, 1H), 7.84 (s, 1H), 7.39-7.22 (m, 7H), 6.72 (s, 2H), 6.22 (d, J=2.2 Hz, 1H), 5.50 (dd, J=6.3, 2.2 Hz, 1H), 5.02 (d, J=9.6 Hz, 3H), 4.40 (ddd, J=8.4, 5.7, 2.8 Hz, 1H), 3.51-3.37 (m, 8H), 3.28 (t, J=6.7 Hz, 3H), 3.20-3.02 (m, 4H), 2.81 (t, J=7.7 Hz, 2H), 1.70-1.12 (m, 14H), 1.06 (dq, J=15.0, 6.8 Hz, 2H), 1.00-0.88 (m, 2H). ¹³C NMR (101 MHz, DMSO) δ 156.19, 154.77, 150.97, 148.42, 141.02, 137.20, 128.36, 127.79, 127.75, 119.21, 113.21, 89.40, 85.91, 83.10, 82.25, 70.26, 69.57. 69.42, 69.38, 69.13, 65.24, 50.19, 49.76, 40.19, 29.11, 27.25, 26.89, 25.98, 25.28, 25.16. LC-MS (C₃₁H₄₆N₈O₉S): Calculated [M+H]⁺ m/z=707.32, [M−H]⁻ m/z=705.30. Observed [M+H]⁺ m/z=706.92, [M−H]⁻ m/z=705.41.

### tert-butyl (S)-2-((N-(((3aR,4R,6R,6aR)-6-(6-amino-9H-purin-9-yl)-2,2-dimethyltetrahydrofuro[3,4-d][1,3]dioxol-4-yl)methyl)-N-(3-oxo-1-phenyl-2,7,10-trioxa-4-azahexadecan-16-yl)sulfamoyl)carbamoyl)pyrrolidine-1-carboxylate (MAT566)

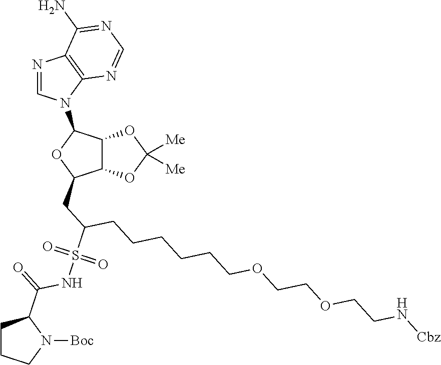 [SMILES: CC(C)(C)OC(=O)N1CCC[C@H]1C(=O)NS(=O)(=O)C(CCCCCCOCCOCCNC(=O)OCc1ccccc1)C[C@H]1O[C@@H](n2cnc3c(N)ncnc32)[C@@H]2OC(C)(C)O[C@@H]21]

Prepared crude MAT564 (481 mg theoretical yield) as described above, but did not quench, concentrate, or otherwise purify (i.e. still contained MAT15. DBU, and MeCN).

In a separate vial, dissolved BOC-L-proline N-hydroxysuccinimide ester (716 mg, 2.29 mmol, 3.4 eq) in MeCN (4 mL) and then added DBU (500 μL, 505 mg, 3.32 mmol, 4.87 eq). Stirred for 10 minutes. Added the MAT564 reaction to this solution and stirred vigorously for 2.5 h. Concentrated to remove most, but not all, MeCN (˜5-10 mL remaining). Diluted with mixture of saturated NH₄Cl_{(aq)} (100 mL), brine (75 mL), 1.0 M HCl (50 mL), and water (50 mL). Extracted twice with DCM (200 mL). Dried over Na₂SO₄, filtered, and concentrated onto silica gel. Purified by flash column chromatography (EtOAc->1:1:1 water/MeOH/MeCN) and then by reverse phase flash column chromatography (water/MeCN). Yield: 260 mg, 42% (from MAT563). Light yellow solid. ¹H NMR (400 MHz, DMSO) δ 11.68 (d, J=14.6 Hz, 1H), 8.37 (s, 1H), 8.20 (d, J=1.5 Hz, 1H), 7.54 (s, 2H), 7.41-7.25 (m, 7H), 6.24 (dd, J=6.9, 2.1 Hz, 1H), 5.49 (td, J=5.8, 2.1 Hz, 1H), 5.06 (dd, J=6.4, 3.1 Hz, 1H), 5.01 (s, 2H), 4.37 (qt, J=6.5, 3.5 Hz, 1H), 4.09 (td, J=8.1, 4.1 Hz, 1H), 3.82 (ddd, J=25.8, 15.4, 4.4 Hz, 1H), 3.49 (dd, J=6.0, 3.6 Hz, 3H), 3.46-3.23 (m, 11H), 3.15 (q, J=5.9 Hz, 2H), 3.01 (dt, J=15.8, 6.7 Hz, 1H), 2.93-2.77 (m, 1H), 2.55 (s, 2H), 2.18-2.04 (m, 1H), 1.83-1.69 (m, 3H), 1.54 (s, 3H), 1.51-1.42 (m, 1H), 1.39 (d, J=6.5 Hz, 4H), 1.32 (d, J=6.3 Hz, 11H), 1.26 (d. J=17.1 Hz, 1H), 1.19-1.04 (m, 2H), 0.99 (t, J=7.8 Hz, 2H), 0.84 (dq, J=15.1, 7.0 Hz, 2H). ¹³C NMR (101 MHz, DMSO) δ 171.59, 171.26, 156.18, 155.75, 153.42, 152.87, 152.13, 148.44, 148.40, 140.66, 137.20, 128.36, 127.79, 127.75, 119.26, 113.35, 113.28, 89.42, 89.27, 86.23, 85.88, 83.22, 82.19, 82.10, 78.94, 78.91, 70.21, 70.18, 69.57, 69.37, 69.13, 65.24, 59.21, 50.60, 49.89, 49.79, 46.50, 46.34, 40.43, 30.50, 29.58, 29.04, 29.00, 28.10, 27.87, 27.84, 26.97, 26.92, 26.89, 25.78, 25.71, 25.16, 25.12, 23.84, 23.06. LC-MS (C₄₁H₆₁N₉O₁₂S): Calculated [M+H]⁻ m/z=904.42, [M−H]⁻ m/z=902.41. Observed [M+H]⁺ m/z=903.96, [M−H]⁻ m/z=902.52.

### tert-butyl (S)-2-((N-(((3aR,4R,6R,6aR)-6-(6-amino-9H-purin-9-yl)-2,2-dimethyltetrahydrofuro[3,4-d][1,3]dioxo-4-y)methyl)-N-(6-(2-(2-aminoethoxy)ethoxy)hexyl)sulfamoyl)carbamoyl)pyrrolidine-1-carboxylate (MAT567)

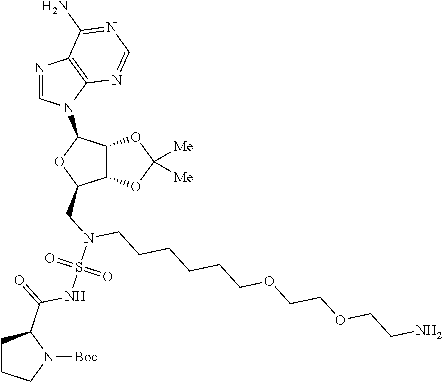 [SMILES: CC(C)(C)OC(=O)N1CCC[C@H]1C(=O)NS(=O)(=O)N(CCCCCCOCCOCCN)C[C@H]1O[C@@H](n2cnc3c(N)ncnc32)[C@@H]2OC(C)(C)O[C@@H]21]

Charged vial with 10% wt palladium on carbon (Pd/C; 6.3 mg, 5.9 μmol, 0.12 eq) and AcOH (2 mL) and stirred vigorously. To this, added a solution of MAT566 (43.7 mg, 48.3 μmol, 1 eq) in AcOH (1 mL). Sparged with hydrogen gas until reaction was complete by LC-MS. Filtered to remove Pd/C and concentrated in vacuo to obtain MAT567 as the acetic acid salt. Yield: 32.5 mg, 79.3%. Off-white solid (slight hint of yellow). ¹H NMR (400 MHz, DMSO) δ 8.33 (s, 1H), 8.16 (s, 1H), 7.35 (d, J=6.9 Hz, 2H), 6.19 (dd, J=5.5, 2.2 Hz, 1H), 5.51 (dt, J=5.2, 2.5 Hz, 1H), 5.08-4.99 (m, 1H), 4.39 (tt, J=8.3, 3.5 Hz, 1H), 3.98 (ddt, J=18.4, 10.6, 5.0 Hz, 1H), 3.68 (dd, J=15.1, 5.2 Hz, 1H), 3.60 (t, J=5.3 Hz, 2H), 3.56 (dd, J=5.9, 3.4 Hz, 2H), 3.52-3.36 (m, 4H), 3.36-3.10 (m, 6H), 2.97 (t, J=5.3 Hz, 2H), 2.83 (qd, J=15.2, 7.8 Hz, 2H), 2.55 (s, 1H), 2.14-1.95 (m, 1H), 1.91 (s, 4H), 1.74 (h, J=6.0 Hz, 3H), 1.53 (s, 3H), 1.46 (d, J=17.0 Hz, 1H), 1.41-1.27 (m, 15H), 1.13 (q, J=7.2 H-z, 2H), 1.03 (p, J=7.4 Hz, 3H), 0.89 (p, J=7.2 Hz, 2H). ¹³C NMR (101 MHz, DMSO) δ 172.09, 156.20, 152.68, 148.62, 140.36, 128.36, 119.24, 113.12, 113.07, 89.35, 83.02, 82.32, 78.30, 70.20, 69.80, 69.35, 66.81, 46.27, 40.43, 38.70, 30.81, 29.01, 28.21, 27.95, 26.93, 25.83, 25.19, 22.95, 21.14. LC-MS (C₃₃H₅₅N₉O₁₀S): Calculated [M+H]⁺ m/z=770.39, [M−H]⁻ m/z=768.37. Observed [M+H]⁺ m/z=770.48, [M−H]⁻ m/z=768.66

### 4-((2-(2-((6-((((3aR,4R,6R,6aR)-6-(6-amino-9H-purin-9-yl)-2,2-dimethyltetrahydrofuro[3,4-d][1,3]dioxol-4-yl)methyl)(N-((tert-butoxycarbonyl)-L-propyl))sulfamoyl)amino)hexyl)oxy)ethoxy)ethyl)carbamoyl)-2-(6-hydroxy-3-oxo-3H-xanthen-9-yl)benzoic acid (M4T573)

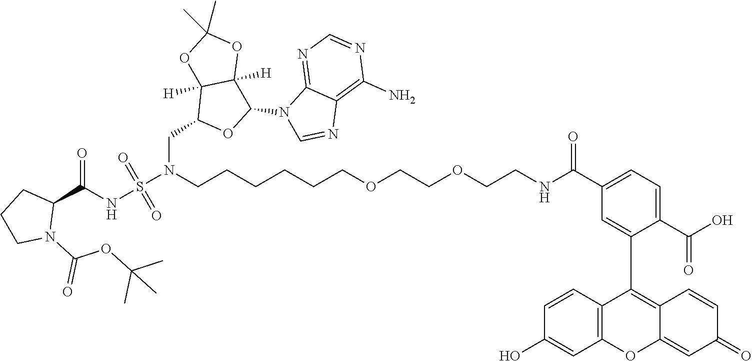 [SMILES: CC(C)(C)OC(=O)N1CCC[C@H]1C(=O)NS(=O)(=O)N(CCCCCCOCCOCCNC(=O)c1ccc(C(=O)O)c(-c2c3ccc(=O)cc-3oc3cc(O)ccc23)c1)C[C@H]1O[C@@H](n2cnc3c(N)ncnc32)[C@@H]2OC(C)(C)O[C@@H]21]

Charged vial with MAT567 (10.8 mg, 12.7 μmol, 1 eq) and DMSO (200 μL) and stirred. To this, successively added DIPEA (20.0 μL, 14.8 mg, 115 μmol, 9 eq) and then a solution of 5/6-carboxyfluorescein succinimidyl ester (7.6 mg, 16 μmol, 1.2 eq) in DMF (1 mL). Stirred for 1.5 h. Purified reaction mixture directly by reverse phase flash column chromatography (water/MeCN) and then repurified by reverse phase flash column chromatography (water/MeCN, both with 0.1% formic acid). Yield: 7.6 mg, 53%. Orange solid. !H NMR (400 MHz, MeOD) δ 8.43 (s, 2H), 8.37 (d, J=6.5 Hz, 1H), 8.22 (dd, J=8.0, 1.6 Hz, 1H), 8.19-8.05 (m, 2H), 7.33 (t, J=8.2 Hz, 1H), 6.70 (dd, J=6.9, 2.3 Hz, 2H), 6.59 (ddd, J=20.3, 10.1, 7.2 Hz, 4H), 6.27 (dd, J=8.1, 1.9 Hz, 1H), 5.52-5.43 (m, 1H), 5.12-5.05 (m, 1H), 4.49-4.43 (m, 1H), 4.11 (t, J=7.1 Hz, 1H), 3.75-3.59 (m, 7H), 3.59-3.35 (m, 9H), 3.03 (dt, J=15.8, 6.8 Hz, 1H), 2.71 (s, 1H), 2.66 (s, 1H), 1.90-1.83 (m, 3H), 1.58 (d, J=6.0 Hz, 3H), 1.42 (t, J=5.1 Hz, 11H), 1.39-1.27 (m, 12H), 1.09 (s, 2H). ¹³C NMR (101 MHz, MeOD) δ 163.03, 160.44, 154.28, 152.88, 136.48, 134.12, 129.04, 128.89, 124.53, 123.70, 114.23, 112.58, 109.70, 102.21, 90.76, 84.03, 82.54, 80.55, 79.97, 70.82, 69.98, 69.76, 69.62, 69.07, 68.91, 59.85, 58.33, 51.13, 46.67, 46.30, 39.82, 39.02, 34.49, 30.67, 29.55, 29.10, 28.97, 27.52, 27.33, 27.18, 25.98, 25.35, 24.06, 23.93, 23.16. Note: ¹³C NMR spectra had insufficient signal to background to see all expected resonances but we have listed what was observed. LC-MS (C₅₄H₆₅N₉O₁₆S): Calculated [M+H]⁺ m/z=1128.43, [M−H]⁻ m/z=1126.42. Observed [M+H]⁺ m/z=1128.52, [M−H]⁻ m/z=1126.76.

### 4-((2-(2-((6-((N-(L-prolyl)sulfamoyl)(((2R,3S4R,5R)-5-(6-amino-9H-purin-9-yl)-3,4-dihydroxytetrahydrofuran-2-yl)methyl)amino)hexyl)oxy)ethoxy)ethyl)carbamoyl)-2-(6-hydroxy-3-oxo-3H-xanthen-9-yl)benzoic acid (MAT574)

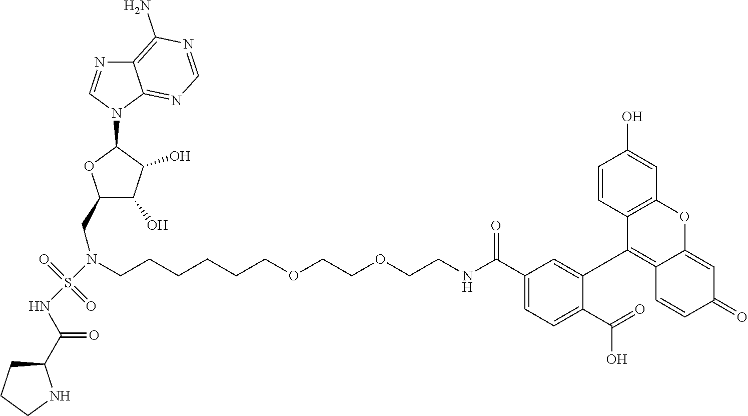 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H]2CCCN2)[C@@H](O)[C@H]1O]

To a vial containing MAT573 (7.6 mg, 6.7 μmol, 1 eq), added solution of 5:2 TFA/water (1.2 mL). Stirred vigorously for 15 minutes. Concentrated in vacuo and azeotroped 3× with MeOH (2 mL). Purified by reverse phase flash column chromatography (water/MeCN). Yield: 3.9 mg, 58% (including 4 equivalents of N-hydroxysuccinimide). Orange solid. ¹H NMR (400 MHz, DMSO) δ 8.81 (s, 1H), 8.48 (s, 1H), 8.33 (s, 1H), 8.09 (d, J=27.5 Hz, 2H), 7.40-7.14 (m, 3H), 6.61 (d, J=8.9 Hz, 2H), 6.54-6.23 (m, 4H), 5.83 (d, J=6.2 Hz, 1H), 4.74 (d, J=6.5 Hz, 1H), 4.19-4.02 (m, 2H), 3.89-2.82 (m, 120H), 2.09-1.95 (m, 1H), 1.83 (s, 2H), 1.69 (dd. J=14.2, 7.1 Hz, 2H), 1.49-0.81 (m, 10H). LC-MS (C₄₆H₅₃N₉O₁₄S): Calculated [M+H]⁺ m/z=988.35, [M−H]⁻ m/z=986.34. Observed [M+H]⁺ m/z=988.80, [M−H]⁻ m/z=986.69.

### tert-butyl ((S)-((N-(((3aR,4R,6R,6aR)-6-(6-amino-9H-purin-9-yl)-2,2-dimethyltetrahydrofuro[3,4-d][1,3]dioxol-4-yl)methyl)-N-(3-oxo-1-phenyl-2,7,10-trioxa-4-azahexadecan-16-yl)sulfamoyl)amino)-1-oxo-3-phenylpropan-2-yl)carbamate (M4T570)

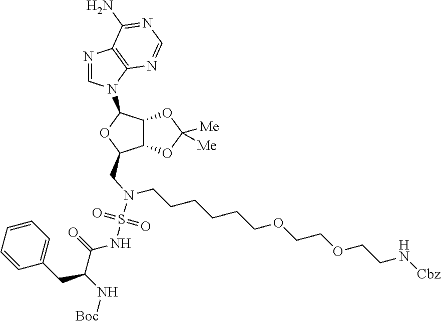 [SMILES: CC(C)(C)OC(=O)N[C@@H](Cc1ccccc1)C(=O)NS(=O)(=O)N(CCCCCCOCCOCCNC(=O)OCc1ccccc1)C[C@H]1O[C@@H](n2cnc3c(N)ncnc32)[C@@H]2OC(C)(C)O[C@@H]21]

Prepared using the general protocol described for MAT566, except the BOC-L-proline N-hydroxysuccinimide ester was replaced with BOC-L-phenylalanine N-hydroxysuccinimide ester Yield: 122 mg, 17%. White solid. ¹H NMR (400 MHz, DMSO) δ 11.80 (d, J=10.8 Hz, 1H), 8.36 (s, 1H), 8.20 (d, J=6.6 Hz, 1H), 7.66-7.47 (m, 2H), 7.40-7.22 (m, 10H), 7.17 (p, J=6.8 Hz, 2H), 6.23 (d, J=2.0 Hz, 1H), 5.47 (dt, J=5.4, 2.5 Hz, 1H), 5.07-5.02 (m, 1H), 5.00 (s, 2H), 4.37 (tt J=8.8, 3.7 Hz, 1H), 4.15 (ddt, J=13.5, 7.1, 3.1 Hz, 1H), 3.80 (ddd, J=15.6, 11.2, 4.5 Hz, 1H), 3.50-3.27 (m, 9H), 3.27-3.21 (m, 2H), 3.14 (q, J=5.9 Hz, 2H), 2.87 (qd, J=10.8. 6.1 Hz, 3H), 2.68 (td, J=11.3, 5.8 Hz, 1H), 1.52 (d, J=8.3 Hz, 3H), 1.36-1.20 (m, 14H), 1.20-1.03 (m, 2H), 0.97 (p, J=8.4 Hz, 2H), 0.84 (p, J=7.6 Hz, 2H). ¹³C NMR (101 MHz, DMSO) δ 171.50, 171.36, 156.18, 155.64, 155.43, 151.96, 148.41, 140.69, 137.52, 137.49, 137.20, 129.30, 129.27, 128.36, 128.07, 127.78, 127.74, 126.42, 119.27, 113.33, 89.31, 86.14, 85.99, 83.25, 83.19, 82.12, 78.30, 70.24, 70.20, 69.55, 69.36, 69.12, 65.23, 56.03, 50.66, 49.82, 36.34, 29.07, 29.02, 28.11, 28.07, 27.73, 26.91, 26.64, 25.86, 25.76, 25.17, 25.12, 25.08. LC-MS (C₄₅H₆₃N₉O₁₂S): Calculated [M+H]⁺ m/z=954.44, [M−H]⁻ m/z=952.42. Observed [M+H]⁺ m/z=954.01, [M−H]⁻ m/z=952.56.

### tert-butyl ((S)-1-((N-(((3aR,4R,6R,6aR)-6-(6-amino-9H-purin-9-yl)-2,2-dimethyltetrahydrofuro[3,4-d][1,3]dioxol-4-yl)methyl)-N-(6-(2-(2-aminoethoxy)ethoxy)hexyl)sulfamoyl)amino)-1-oxo-3-phenylpropan-2-yl)carbamate (MAT571)

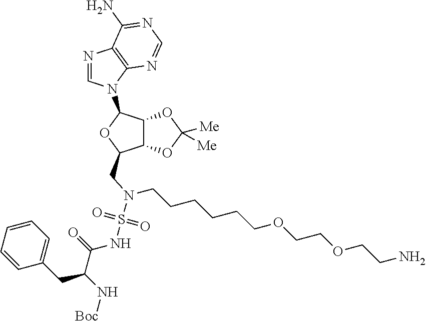 [SMILES: CC(C)(C)OC(=O)N[C@@H](Cc1ccccc1)C(=O)NS(=O)(=O)N(CCCCCCOCCOCCN)C[C@H]1O[C@@H](n2cnc3c(N)ncnc32)[C@@H]2OC(C)(C)O[C@@H]21]

Charged vial with 10% wt palladium on carbon (Pd/C; 6.5 mg, 6.1 μmol, 0.17 eq) and AcOH (1 mL) and stirred vigorously. To this, added a solution of MAT570 (34.7 mg, 36.4 μmol, 1 eq) in AcOH (2 mL). Sparged with hydrogen gas until s reaction stopped by LC-MS. Filtered to remove Pd/C and concentrated in vacuo. Purified by reverse phase flash column chromatography (water/MeCN, both with 0.1% formic acid). Yield: 22.6 mg, 75.8%. Off-white solid (slightly tan). ¹H NMR (400 MHz, DMSO) δ 8.34 (s, 1H), 8.24 (s, 2H), 8.17 (d, J=2.1 Hz, 1H), 7.37 (s, 2H), 7.26-7.12 (m, 5H), 6.58-6.47 (m, 1H), 6.19 (d, J=2.3 Hz, 1H), 5.51 (dt, J=6.0, 2.3 Hz, 1H), 5.04 (dd, J=6.3, 2.8 Hz, 1H), 4.45-4.34 (m, 1H), 4.00 (tt, J=9.8, 5.0 Hz, 1H), 3.67 (ddd, J=15.0, 10.1, 5.3 Hz, 1H), 3.59 (t, J=5.1 Hz, 2H), 3.57-3.51 (m, 2H), 3.47 (t, J=4.4 Hz, 2H), 3.28 (t, J=6.6 Hz, 2H), 3.16 (td, J=15.6, 8.0 Hz, 1H), 3.00-2.91 (m, 3H), 2.87-2.69 (m, 3H), 1.52 (d, J=3.6 Hz, 3H), 1.31 (d, J=11.1 Hz, 13H), 1.21 (d, J=3.8 Hz, 2H), 1.14 (h, J=5.6 Hz, 2H), 1.02 (q, J=7.6 Hz, 2H), 0.96-0.84 (m, 2H). ¹³C NMR (101 MHz, DMSO) δ 173.10, 173.03, 164.03, 156.21, 155.04, 155.01, 152.69, 148.68, 140.32, 138.25, 138.20, 129.40, 129.37, 127.89, 126.06, 119.23, 113.09, 89.28, 86.28, 86.15, 83.01, 82.98, 82.30, 77.85, 70.23, 69.79, 69.33, 66.81, 56.63, 51.09, 50.91, 49.97, 40.43, 38.66, 37.37, 29.05, 29.03, 28.17, 27.80, 27.28, 27.16, 26.95, 25.93, 25.88, 25.21. LC-MS (C₃₇H₅₇N₉O₁₀S): Calculated [M+H]⁺ m/z=820.40, [M−H]⁻ m/z=818.39. Observed [M+H]⁻ m/z=820.70, [M−H]⁻ m/z=818.54.

### (S)-2-amino-N-(N-(((2R,3S,4R,5R)-S-(6-amino-9H-purin-9-yl)-3,4-dihydroxytetrahydrofuran-2-yl)methyl)-N-(6-(2-(2-aminoethoxy)ethoxy)hexyl)sulfamoyl)-3-phenylpropanamide (MAT581)

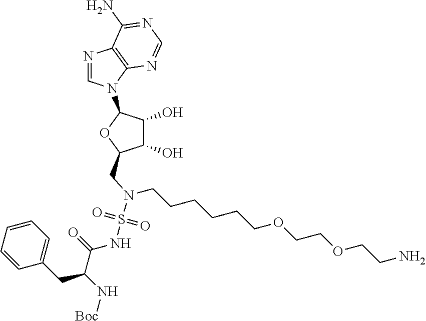 [SMILES: CC(C)(C)OC(=O)N[C@@H](Cc1ccccc1)C(=O)NS(=O)(=O)N(CCCCCCOCCOCCN)C[C@H]1O[C@@H](n2cnc3c(N)ncnc32)[C@H](O)[C@@H]1O]

To the formic acid salt of MAT571 (18.1 mg, 20.9 μmol, 1 eq), added mixture of 5:1 TFA/water (2.4 mL). Stirred vigorously for 20 minutes. Concentrated in vacuo and azeotroped 3× with EtOH (2 mL). Purified by reverse phase flash column chromatography (water/“MeCN+0.1% formic acid”). Yield: 7.3 mg mg, 51%. ¹H NMR (400 MHz, DMSO) δ 8.33 (d, J=3.4 Hz, 1H), 8.13 (s, 1H), 7.27 (dd, J=4.5, 1.9 Hz, 6H), 7.25-7.16 (m, 2H), 5.85 (d, J=6.3 Hz, 1H), 4.80 (t, J=5.7 Hz, 1H), 4.19-4.05 (m, 2H), 3.63 (dd, J=15.3, 5.8 Hz, 1H), 3.59-3.41 (m, 9H), 3.27 (t, J=6.6 Hz, 2H), 3.19-2.96 (m, 3H), 2.95-2.78 (m, 4H), 1.36 (h, J=7.1 Hz, 4H), 1.12-0.98 (m, 4H). LC-MS (C₂₉H₄₅N₉O₈S): Calculated [M+H]⁺ m/z=680.32, [M−H]⁻ m/z=678.30. Observed [M+H]⁺ m/z=680.68, [M−H]− m/z=678.58. 4-((2-(2-((6-((((3aR,4R,6R,6aR)-6-(6-amino-9H-purin-9-yl)-2,2-dimethyltetrahydrofuro[3,4-d][1,3]dioxol-4-yl)methyl)(N-((tert-butoxycarbonyl)-L-phenylalanyl)sulfamoy,l)amno)hexyl)oxy)ethoxy)ethyl)carbamoyl)-2-(6-hydroxy-3-oxo-3H-xanthen-9-yl)benzoic acid (MAT587)

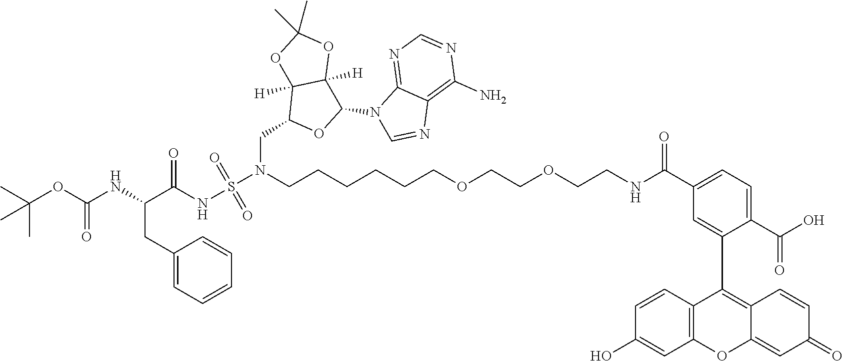 [SMILES: CC(C)(C)OC(=O)N[C@@H](Cc1ccccc1)C(=O)NS(=O)(=O)N(CCCCCCOCCOCCNC(=O)c1ccc(C(=O)O)c(-c2c3ccc(=O)cc-3oc3cc(O)ccc23)c1)C[C@H]1O[C@@H](n2cnc3c(N)ncnc32)[C@@H]2OC(C)(C)O[C@@H]21]

Charged vial with MAT571 (10.1 mg, 12.3 μmol, 1 eq) and DMSO (600 μL) and stirred. To this, successively added DIPEA (20 μL, 15 mg, 0.11 mmol, 9.3 eq) and then a solution of 5/6-carboxyfluorescein succinimidyl ester (8.2 mg, 17 μmol, 1.4 eq) in DMF (0.6 mL). Stirred for 2 h. Purified reaction mixture directly by reverse phase flash column chromatography (water/MeCN, both with 0.1% formic acid). Yield: 11.1 mg, 76.5%. Orange solid. ¹H NMR (400 MHz, DMSO) δ 11.77 (s, 1H), 10.16 (s, 2H), 8.90 (t, J=5.5 Hz, 1H), 8.46 (s, 1H), 8.32 (d, J=2.9 Hz, 1H), 8.16 (ddd, J=8.6, 5.8, 2.2 Hz, 1H), 7.42-7.33 (m, 2H), 7.33-7.21 (m, 4H), 7.21-7.03 (m, 2H), 6.69 (t, J=2.5 Hz, 2H), 6.61-6.49 (m, 3H), 6.22 (d, J=2.2 Hz, 11H), 5.53-5.44 (m, 1H), 5.09-4.97 (m, 1H), 4.36 (s, 1H), 4.12 (s, 1H), 3.77 (d, J=13.2 Hz, 1H), 3.66-3.50 (m, 3H), 3.46 (qd, J=7.4, 5.2 Hz, 4H), 3.37 (d, J=6.0 Hz, 2H), 3.27 (d, J=6.7 Hz, 2H), 3.23-3.15 (m, 1H), 2.88 (d, J=15.3 Hz, 3H), 2.70 (d, J=17.8 Hz, 2H), 1.52 (d, J=8.3 Hz, 3H), 1.39-1.20 (m, 16H), 1.20-1.01 (m, 4H), 1.01-0.89 (m, 2H), 0.84 (d, J=10.6 Hz, 2H). ¹³C NMR (101 MHz, DMSO) δ 168.19, 159.62, 156.22. 152.68, 151.83, 148.50, 136.15, 129.27, 128.02, 126.48, 119.28, 113.25, 112.68, 109.08, 102.26, 70.21, 69.58, 48.61, 29.02, 28.10, 28.07, 26.91, 25.51, 25.16. LC-MS (C₅₈H₆₇N₉O₁₆S): Calculated [M+H]⁻ m/z=1178.45, [M−H]⁻ m/z=1176.44. Observed [M+H]⁺ m/z=1178.33, [M−H]⁻ m/z=1176.61.

### 4-((2-(2-((6-((N-(L-phenylalanyl)sulfamoyl)(((2R,3S,4R,5R)-5-(6-amino-9H-purin-9-yl)-3,4-dihydroxytetrahydrofuran-2-yl)methyl)amino)hexyl)oxy)ethoxy)ethyl)carbamoyl)-2-(6-hydroxy-3-oxo-3H-xanthen-9-yl)benzoic acid (MAT588)

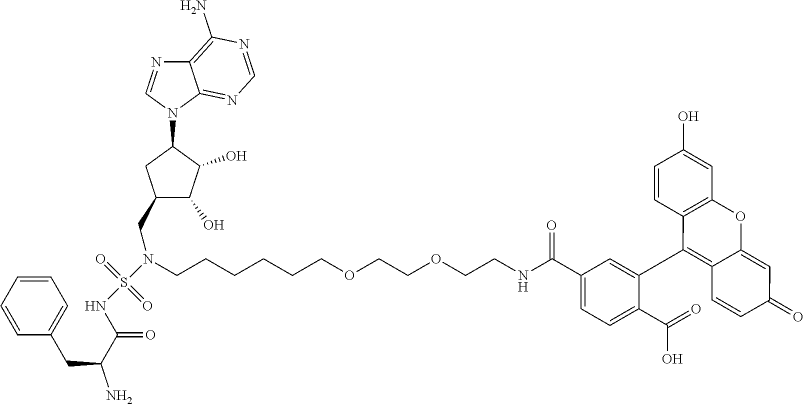 [SMILES: Nc1ncnc2c1ncn2[C@@H]1C[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H](N)Cc2ccccc2)[C@@H](O)[C@H]1O]

To MAT587 (6.7 mg, 5.7 μmol, 1 eq), added mixture of 5:1 TFA/water (2.4 mL). Stirred vigorously for 15 minutes. Concentrated in vacuo and azeotroped 3× with EtOH (2 mL). Purified by reverse phase flash column chromatography (water/MeCN). Azeotroped with once with d₆-benzene. Orange solid. ¹H NMR (400 MHz, DMSO) δ 8.91 (s, 1H), 8.46 (s, 1H), 8.32 (d, J=3.4 Hz, 1H), 8.27-8.02 (m, 6H), 7.36 (dd, J=8.1, 3.9 Hz, 1H), 7.32-7.18 (m, 7H), 7.16 (s, 1H), 6.73-6.65 (m, 2H), 6.62-6.51 (m, 4H), 5.84 (d, J=6.6 Hz, 1H), 4.80 (d, J=6.3 Hz, 1H), 4.34 (s, 1H), 4.15 (s, 1H), 4.09 (s, 1H), 3.96-2.74 (m, 51H), 1.32 (s, 5H), 1.23 (s, 1H), 1.03 (s, 5H). ¹³C NMR (101 MHz, DMSO) δ 151.85, 102.28. LC-MS (C₅₀H₅₅N₉O₁₄S): Calculated [M+H]⁺ m/z=1038.37, [M−H]⁻ m/z=1036.35. Observed [M+H]⁺ m/z=1038.68, [M−H]⁻ m/z=1036.49.

### tert-butyl (S)-5-((N-(((3aR,4R,6R,6aR)-6-(6-amino-9H-purin-9-yl)-2,2-dimethyltetrahydrofuro[3,4-d][1,3]dioxol-4-yl)methyl)-N-(3-oxo-1-phenyl-2,7,10-trioxa-4-azahexadecan-16-yl)sulfamoyl)amino)-4-((tert-butoxycarbonyl)amino)-5-oxopentanoate (M41568)

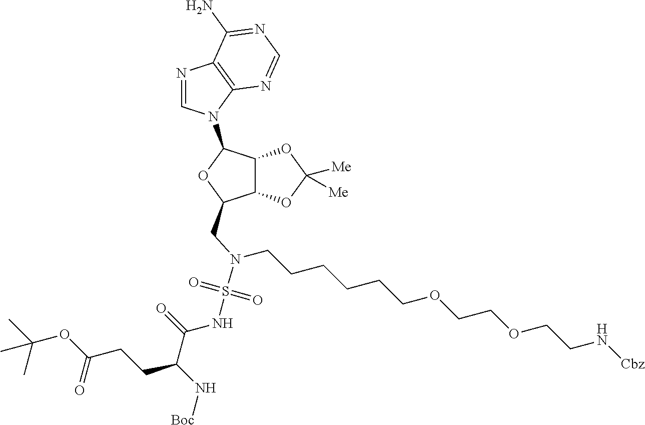 [SMILES: CC(C)(C)OC(=O)CC[C@H](NC(=O)OC(C)(C)C)C(=O)NS(=O)(=O)N(CCCCCCOCCOCCNC(=O)OCc1ccccc1)C[C@H]1O[C@@H](n2cnc3c(N)ncnc32)[C@@H]2OC(C)(C)O[C@@H]21]

Prepared crude MAT564 (532 mg theoretical yield) as described above, but did not quench, concentrate, or otherwise purify (i.e. still contained MAT15, DBU, and MeCN). In a separate vial, dissolved N-BOC-O5-tert-butyl-L-glutamic acid N-hydroxysuccinimide ester (606 mg, 1.51 mmol, 2 eq) in MeCN (5 mL) and then added DBU (600 μL, 606 mg, 3.98 mmol, 5.29 eq). Stirred for 10 minutes. Added the MAT564 reaction to this solution and stirred vigorously for 16 h. Concentrated to remove most, but not all, MeCN (˜5-10 mL remaining). Purified by reverse phase flash column chromatography (loaded with 1M NaOAc, pH 4; eluted with “water/MeCN, both with 0.1% formic acid”). Yield: 3.11 mg, 42% (from MAT563). White solid. ¹H NMR (400 MHz, DMSO) δ 11.63 (s, 1H), 8.38 (s, 1H), 8.21 (d, J=3.2 Hz, 1H), 7.68 (s, 2H), 7.39-7.20 (m, 6H), 7.09 (d, J=7.8 Hz, 1H), 6.23 (d, J=2.2 Hz, 1H), 5.46 (dd, J=6.2, 2.2 Hz, 1H), 5.00 (s, 3H), 4.36 (dt, J=9.0, 4.1 Hz, 1H), 3.97-3.71 (m, 2H), 3.52-3.19 (m, 10H), 3.14 (q, J=6.0 Hz, 2H), 2.92 (tt, J=14.9, 6.0 Hz, 2H), 2.20 (t, J=8.0 Hz, 2H), 1.86-0.75 (m, 39H). ¹³C NMR (101 MHz, DMSO) δ 171.50, 171.36, 171.32, 156.18, 155.35, 155.22, 151.48, 148.38, 140.80, 137.20, 128.35, 127.77, 127.73, 127.39, 119.25, 113.40, 113.38, 89.28, 86.06, 85.81, 83.22, 82.03, 79.85, 79.83, 78.37, 70.22, 69.56, 69.37, 69.13, 65.23, 53.75, 50.66, 49.79, 40.18, 31.42, 29.06, 28.13, 28.10, 27.73, 26.91, 26.59, 26.28, 25.82, 25.17, 25.10. LC-MS (C₄₅H₆₉N₉O₁₄S): Calculated [M+H]⁺ m/z=992.48, [M−H]⁻ m/z=990.46. Observed [M+H]⁺ m/z=992.00, [M−H]⁻ m/z=990.65.

Tert-butyl (S)-5-((N-(((3aR,4R,66R,6aR)-6-(6-amino-9H-purin-9-yl)-2,2-dimethyltetrahydrofuro[3,4-d][1,3]dioxol-4-yl)methyl)-N-(6-(2-(2-amino ethoxy)ethoxy)hexyl)sulfamoyl)amino)-4-((tert-butoxycarbonyl)amino)-5-oxopentanoate (MAT569)

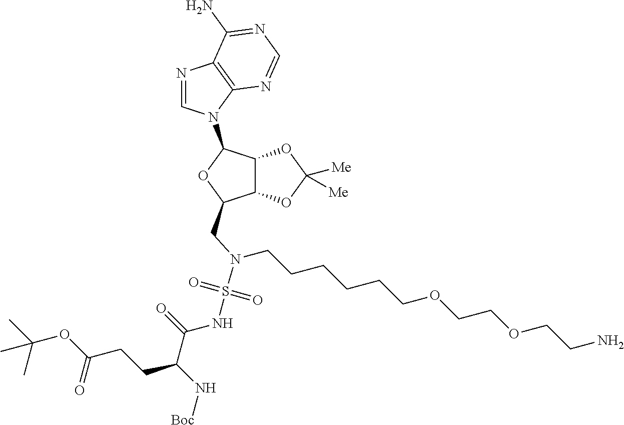 [SMILES: CC(C)(C)OC(=O)CC[C@H](NC(=O)OC(C)(C)C)C(=O)NS(=O)(=O)N(CCCCCCOCCOCCN)C[C@H]1O[C@@H](n2cnc3c(N)ncnc32)[C@@H]2OC(C)(C)O[C@@H]21]

Charged vial with 10% wt palladium on carbon (Pd/C; 7.1 mg, 6.7 μmol, 0.17 eq) and AcOH (1 mL) and stirred vigorously. To this, added a solution of MAT568 (38.4 mg, 38.7 μmol, 1 eq) in AcOH (2 mL). Sparged with hydrogen gas until reaction stopped by LC-MS. Filtered to remove Pd/C and concentrated in vacuo. Purified by reverse phase flash column chromatography (water/MeCN, both with 0.1% formic acid). Yield: 29.8 mg, 89.7%. Off-white solid (light brown tinge). ¹H NMR (400 MHz, DMSO) δ 8.32 (s, 1H), 8.16 (d, J=4.9 Hz, 2H), 7.35 (s, 2H), 6.56 (d, J=7.4 Hz, 1H), 6.18 (d, J=2.3 Hz, 1H), 5.49 (dd, J=6.2, 2.3 Hz, 1H), 5.02 (dd, J=6.3, 2.8 Hz, 1H), 4.37 (ddt, J=10.2, 7.6, 3.7 Hz, 1H), 3.85-3.63 (m, 3H), 3.59 (t, J=5.3 Hz, 2H), 3.55 (dd, J=6.0, 3.5 Hz, 2H), 3.47 (dd, J=5.9, 3.5 Hz, 2H), 3.45-3.39 (m, 1H), 3.27 (d, J=13.0 Hz, 2H), 3.20 (td, J=13.5, 6.1 Hz, 2H), 2.96 (t, J=5.3 Hz, 2H), 2.85 (t, J=8.1 Hz, 2H), 2.54 (s, 1H), 2.17 (d, J=8.2 Hz, 2H), 1.87-1.75 (m, 1H), 1.66 (dq, J=13.8, 7.7 Hz, 1H), 1.52 (s, 3H), 1.41-1.20 (m, 23H), 1.20-1.07 (m, 2H), 1.01 (q, J=8.0 Hz, 2H), 0.89 (p, J=7.7 Hz, 2H). ¹³C NMR (101 MHz, DMSO) δ 172.79, 171.74, 171.63, 163.36, 161.21, 156.19, 155.09, 152.67, 148.65, 140.28, 119.23, 113.18, 113.14, 89.24, 86.16, 85.98, 83.00, 82.23, 79.63, 79.58, 79.35, 79.01, 78.69, 78.01, 70.23, 69.77, 69.57, 69.38, 69.32, 68.96, 66.73, 54.37, 50.82, 49.90, 40.43, 38.63, 37.16, 31.31, 29.04, 28.16, 27.94, 27.75, 27.39, 27.15, 26.95, 25.89, 25.20, 25.17. LC-MS (C₃₇N₉O₁₂S): Calculated [M+H]⁺ m/z=858.44, [M−H]⁻ m/z=856.42. Observed [M+H]⁺ m/z=858.33, [M−H]⁻ m/z=856.71.

### 4-((2-(2-((6-((((3aR,4R,6R,6aR)-6-(6-amino-9H-purin-9-yl)-2,2-dimethyltetrahydrofuro[3,4-d][1,3]dioxol-4-l)methy)(N-((S)-5-(tert-butoxy)-2-((tert-butoxycarbonyl)amino)-S-oxopentanoyl)sulfamoyl)amino)hexyl)oxy)ethoxy)ethyl)carbamoyl)-2-(6-hydroxy-3-oxo-3H-xanthen-9-yl)benzoic acid (MA7T578)

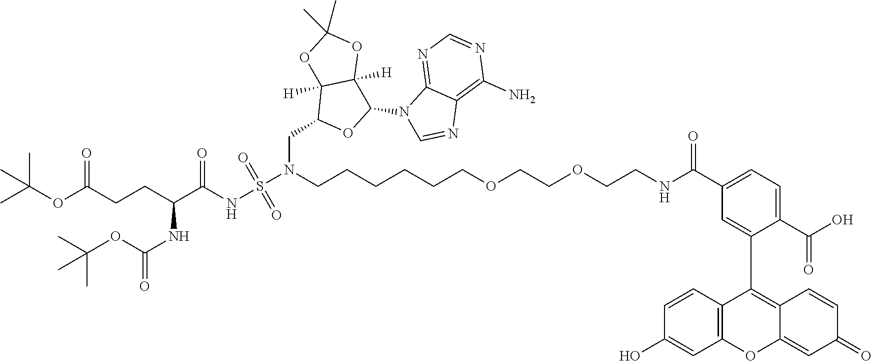 [SMILES: CC(C)(C)OC(=O)CC[C@H](NC(=O)OC(C)(C)C)C(=O)NS(=O)(=O)N(CCCCCCOCCOCCNC(=O)c1ccc(C(=O)O)c(-c2c3ccc(=O)cc-3oc3cc(O)ccc23)c1)C[C@H]1O[C@@H](n2cnc3c(N)ncnc32)[C@@H]2OC(C)(C)O[C@@H]21]

Charged vial with MAT569 (29.8 mg, 34.7 μmol, 1 eq) and DMSO (600 μL) and stirred. To this, successively added DIPEA (60.5 μL, 44.9 mg, 347 μmol, 10 eq) and then a solution of 5/6-carboxyfluorescein succinimidyl ester (22.4 mg, 47.3 μmol, 1.36 eq) in DMF (0.6 mL). Stirred for 22 h. Purified reaction mixture directly by reverse phase flash column chromatography (water/MeCN, both with 0.1% formic acid). Yield: 19.3 mg, 45.7%. Orange solid. ¹H NMR (400 MHz, DMSO) δ 10.16 (s, 2H), 8.91 (t, J=5.5 Hz, 1H), 8.47 (s, 1H), 8.31 (d, J=3.0 Hz, 1H), 8.25 (d, J=8.1 Hz, 1H), 8.15 (d, J=3.6 Hz, 1H), 7.36 (t, J=7.0 Hz, 2H), 6.92 (s, 1H), 6.75-6.44 (m, 6H), 6.20 (s, 1H), 5.48 (d, J=6.3 Hz, 1H), 5.02 (d, J=6.4 Hz, 1H), 4.36 (d, J=8.4 Hz, 1H), 3.93-3.69 (m, 2H), 3.60-3.51 (m, 2H), 3.47 (q, J=6.3 Hz, 4H), 3.40 (d, J=4.4 Hz, 1H), 3.27 (t, J=6.7 Hz, 2H), 3.21 (t, J=6.8 Hz, 1H), 2.89 (s, 2H), 2.18 (t, J=7.9 Hz, 2H), 1.87-1.73 (m, 1H), 1.73-1.60 (m, 1H), 1.52 (s, 3H), 1.41-1.29 (m, 22H), 1.25 (dt, J=16.5, 7.5 Hz, 2H), 1.20-1.04 (m, 2H), 0.99 (q, J=7.6 Hz, 2H), 0.84 (q, J=7.7 Hz, 2H). ¹³C NMR (101 MHz, DMSO) δ 171.43, 168.20, 168.08, 164.72, 164.60, 159.63, 156.22, 155.29, 154.70, 152.69, 151.85, 148.54, 140.57, 140.33, 136.17, 134.72, 129.44, 129.27, 129.18, 128.22, 126.50, 124.88, 124.27, 123.33, 122.32, 119.28, 113.28, 112.75, 112.69, 109.15, 109.10, 102.30, 102.26, 89.28, 86.14, 85.91, 83.29, 83.12, 82.12, 79.76, 78.28, 70.24, 70.17, 69.60, 69.52, 69.40, 69.28, 68.79, 68.65, 53.91, 50.67, 49.85, 31.40, 29.06, 29.02, 28.14, 28.11, 27.73, 26.93, 26.56, 25.85, 25.18, 25.12, 25.10. LC-MS (C₅₈H₇₃N₉O₁₈S): Calculated [M+H]⁺ m/z=1216.49, [M−H]⁻ m/z=1214.47. Observed [M+H]⁺ m/z=1216.30, [M−H]⁻ m/z=1214.59.

### 4-((2-(2-((6-((N-(L-glutamyl)sulfamoyl)(((2R,3S,4R,5R)-5-(6-amino-9H-purin-9-yl)-3,4-dihydroxytetrahydrofuran-2-yl)methyl)amino)hexyl)oxy)ethoxy)ethyl)carbamoyl)-2-(6-hydroxy-3-oxo-3H-xanthen-9-yl)benzoic acid (MAT579)

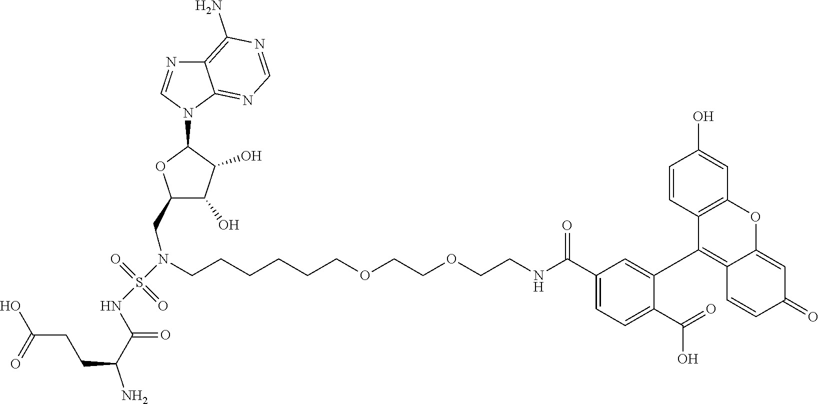 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H](N)CCC(=O)O)[C@@H](O)[C@H]1O]

To MAT578 (19.3 mg, 15.9 μmol, 1 eq), added mixture of 5:1 TFA/water (2.4 mL). Stirred vigorously for 30 minutes. Concentrated in vacuo and azeotroped 3× with MeOH (2 mL). Purified by reverse phase flash column chromatography (water/MeCN). Azeotroped with once with d₆-benzene. Yield: 11.5 mg, 71.1%. Orange solid. ¹H NMR (400 MHz, DMSO) δ 9.56 (s, 3H), 8.84 (dt, J=56.2, 5.6 Hz, 1H), 8.47 (s, 1H), 8.32 (d, J=3.3 Hz, 1H), 8.25 (d, J=7.9 Hz, 1H), 8.19-8.05 (m, 3H), 7.37 (d, J=8.0 Hz, 1H), 7.27 (s, 2H), 6.69 (dd, J=4.4, 2.1 Hz, 2H), 6.63-6.49 (m, 4H), 5.85 (d, J=6.3 Hz, 1H), 4.79 (t, J=5.7 Hz, 1H), 4.15 (t, J=3.8 Hz, 1H), 4.07 (d, J=7.2 Hz, 1H), 3.63 (td, J=13.1, 5.5 Hz, 1H), 3.55 (dt, J=9.6, 5.5 Hz, 4H), 3.46 (h, J=5.9 Hz, 5H), 3.38 (dq, J=15.3, 5.7 Hz, 4H), 3.26 (d, J=13.5 Hz, 2H), 3.20 (t, J=6.7 Hz, 11H), 3.17-3.09 (m, 1H), 3.03 (q, J=6.9 Hz, 1H), 2.86 (p, J=7.5 Hz, 1H), 2.37 (dd, J=18.4, 8.9 Hz, 2H), 1.88 (ddt, J=23.0, 14.9, 7.4 Hz, 2H), 1.49-0.95 (m, 10H). ¹³C NMR (101 MHz, DMSO) δ 173.98, 171.05, 168.23, 168.10, 167.37, 164.73, 164.61, 159.67, 156.08, 154.69, 152.58, 151.85, 149.57, 140.57, 140.02, 136.16, 134.73, 129.28, 129.21, 128.21, 126.51, 124.91, 124.29, 123.35, 122.33. 112.78, 112.72, 109.14, 109.09, 102.30, 87.25, 84.29, 72.27, 71.65, 70.30, 70.23, 69.60, 69.52, 69.38, 69.26, 68.78, 68.64, 56.05, 53.99, 51.99, 50.29, 39.42*, 29.83, 29.14, 29.09, 28.18, 26.82, 26.30, 25.35, 25.32, 18.59. *Peak at 39.42 identified from DEPT135 spectra. LC-MS (C₄₆H₅₃N₉O₆S): Calculated [M+H]⁺ m/z=1020.34, [M−H]⁻ m/z=1018.33. Observed [M+H]⁺ m/z=1020.62, [M−H]⁻ m/z=1018.44.

### Example 4—Biochemical and In Vitro Activity of Selected Compounds

| | Dd2 (EC₅₀ [nM]) | | |
|---|---|---|---|
| | | Halofuginone- | |
| Compound | wt | induced | HFGR-I |
| MAT334 (29) | 76%† | 59%† | 66%† |
| MAT345 (30) | 249 | 7,210 | 7,010 |
| 31* | 102%† | 98%† | 103%† |
| 32 | 63.2 | 1,530 | 2,730 |
| MAT379 (24) | | | |
| | PfcProRS (K_{D} [nM]) | | |
| Compound | — | +proline | +ATP |
| MAT334 (29) | 1,380 | 660 | — |
| MAT345 (30) | 39.8 | 35.9 | — |
| 31* | 2,300 | 1,990 | — |
| 32 | 6.67 | 5.35 | — |
| MAT379 (24) | 100 | | — |
| | HsProRS (K_{D} [nM]) | | |
| Compound | — | +proline | +ATP |
| MAT334 (29) | 2,110 | 2,130 | — |
| MAT345 (30) | 140 | 282 | — |
| 31* | 5.130 | >5,000 | — |
| 32 | 72.1 | 270 | — |
| MAT379 (24) | 1,700 | — | — |

Data are presented as the mean of n≥2 technical replicates and are representative of n ≥3 biologically independent experiments. † Data shown is percent growth at 10 μM because compound had <50% growth inhibition (>50% growth). †† Data shown is percent growth at 1 μM because compound had <50% growth inhibition (>50% growth). * Compound 31 was only tested in 1 bioreplicate (n=6 technical replicates) against each parasite strain.

### Example 5—Assays to Identify and Profile Heterobifunctional Degraders (PROTAC)

### Brief Summary

This example provides a generalizable TR-FRET-based platform to profile the cellular action of heterobifunctional degraders (or PROTACs), capable of both accurately quantifying protein levels in whole cell lysates in less than 1 h and measuring small-molecule target engagement to endogenous proteins. A non-limiting embodiment provided in this example is for human bromodomain-containing protein 4 (BRD4). The detection mix consists of a single primary antibody targeting the protein of interest, a luminescent donor-labeled anti-species nanobody, and a fluorescent acceptor ligand. Importantly, the strategy in this example can readily be applied to other targets of interest and will greatly facilitate the cell-based profiling of small molecule inhibitors and PROTACs in high-throughput format with unmodified cell lines. The platform is validated by exemplary characterization of celastrol, a p-quinone methide-containing pentacyclic triterpenoid, as a broad cysteine-targeting E3 ubiquitin ligase warhead for potent and efficient targeted protein degradation.

### Introduction

Independent of the nature of the POI and the targeted E3 ligase, efficient optimization of PROTACs depends on the availability of robust assay systems that enable the facile, reliable quantification of both biochemical ligand affinities and time- and dose-dependent cellular levels of the POI in response to compound treatment. POI quantification is most commonly done by Western blot analysis, which is inherently time consuming and low throughput. Although various assay technologies, including in-cell Western, enzyme-linked immunosorbent assay (ELISA), AlphaLISA, homogeneous time-resolved fluorescence (HTRF), and luciferase reporter systems, have been developed to increase accuracy and throughput, many depend on the expression of the POI as fusion proteins and/or require expensive specialized equipment and consumables.

A set of complementary assay strategies is described in this example based on a common TR-FRET assay platform that greatly facilitates both the characterization of ligand-target engagement, as well as the quantification of endogenous target protein levels directly in cell lysates in high-throughput format. This approach is employed, by way of non-limiting example, to identify and characterize celastrol, a triterpene natural product that reversibly and covalently binds cysteine side chains, as a powerful E3 ligase recruiter for the development of next generation PROTACs.

### Results

Assay concept and reagent validation: in recent years, assay platforms that combine time-resolved (TR) fluorescence measurements with Förster resonance energy transfer (FRET), often referred to as HTRF immunoassays, have emerged as attractive alternatives to ELISAs and have been successfully employed in PROTAC development for POI quantification and ligand characterization. Similar to sandwich ELISAs. HTRF immunoassays generally employ a matched pair of antibodies for POI quantification, yet do not require antibody immobilization or wash steps (FIG. 21).

TR-FRET assays are also frequently used to determine the affinity of small molecules for respective POIs (FIG. 21). This format generally employs an acceptor-labeled small molecule ligand, referred to as a tracer, in combination with a recombinantly expressed protein featuring an epitope tag (e.g. 6×His, GST or AviTag) that can be TR-FRET donor-functionalized with a corresponding labeled antibody or streptavidin. While the identification of a linker site for tracer development can be difficult, PROTAC development campaigns by default have solved this problem early on. In fact, in recent years, several independent studies have shown the superior performance of TR-FRET based ligand displacement assays for the characterization of PROTAC binding affinities, kinetics, and ternary complex formation.

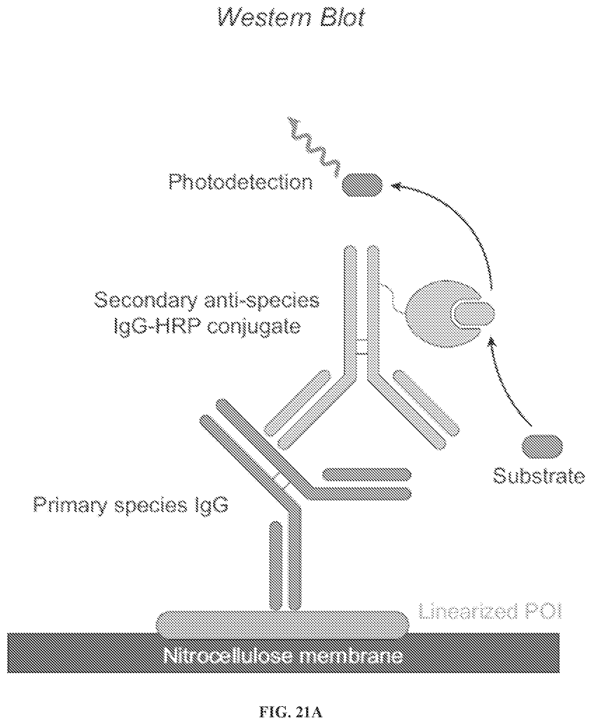

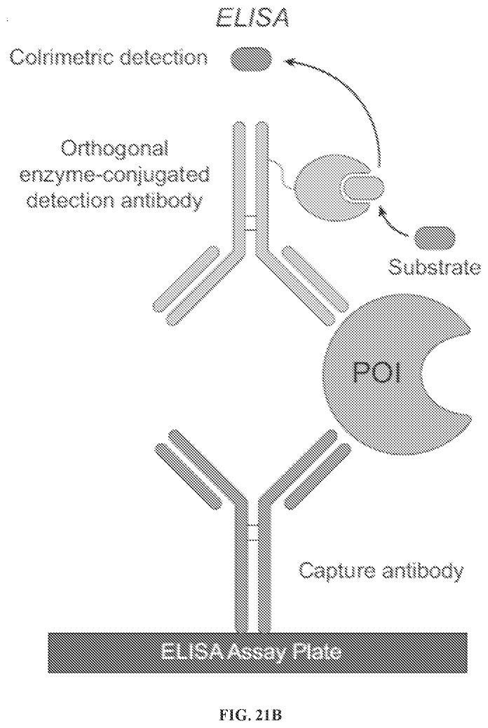

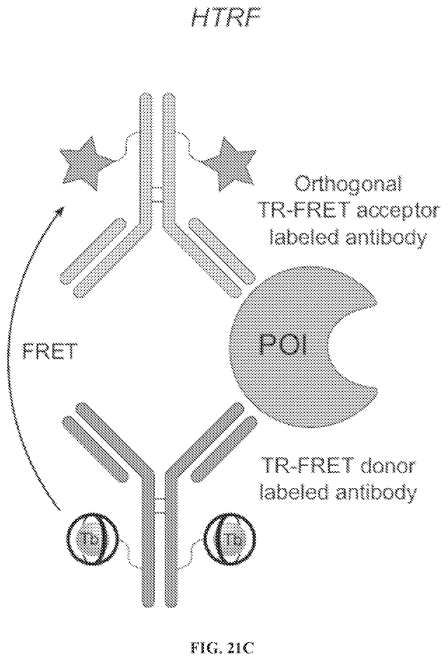

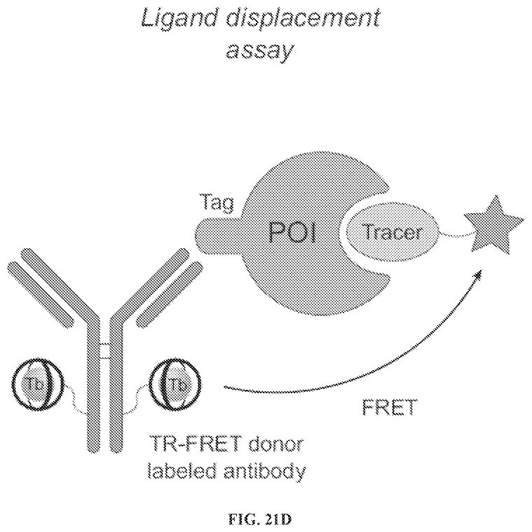

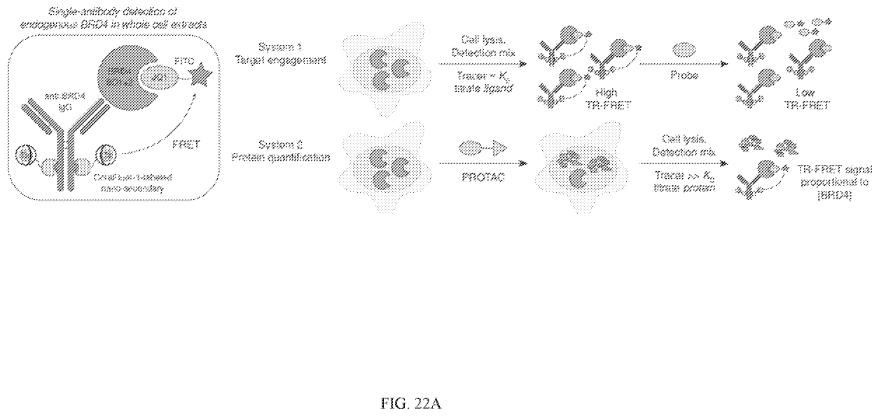

This example shows that rather than utilizing orthogonally labeled matched antibody pairs, which are often difficult to identify, and recombinant POIs, the combination of a tracer with a single antibody directed against the native protein would offer a particularly attractive approach to support PROTAC campaigns by providing a flexible assay platform capable of both ligand affinity profiling and POI quantification directly in cell lysate (FIG. 22A). To eliminate the need for direct covalent labeling of the primary antibody. this approach employs single-domain nanobodies (nano-secondaries), which we labeled with CoraFluor-1, a TR-FRET donor complex with excellent stability and photophysical properties.

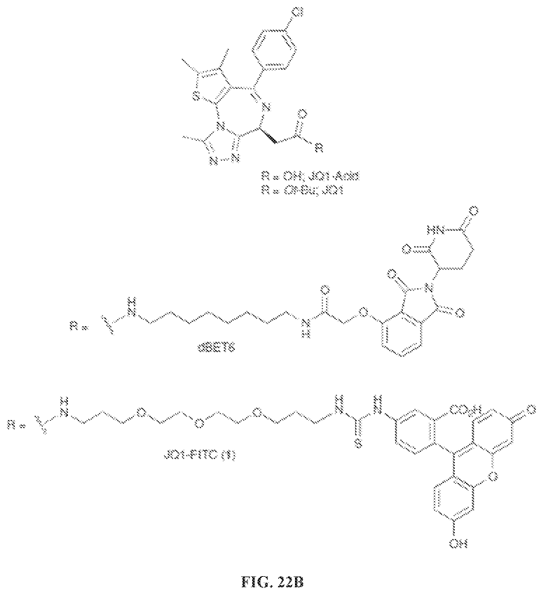

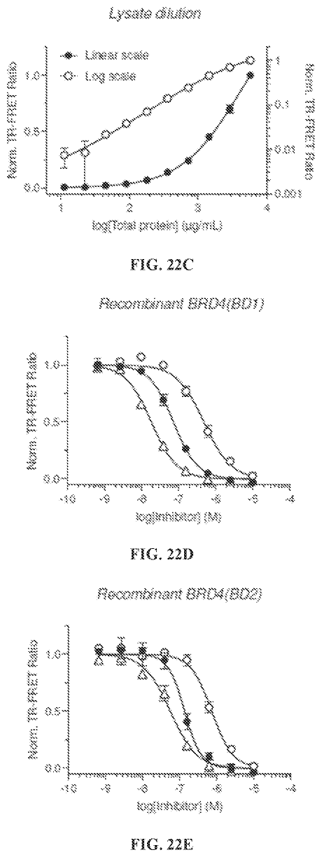

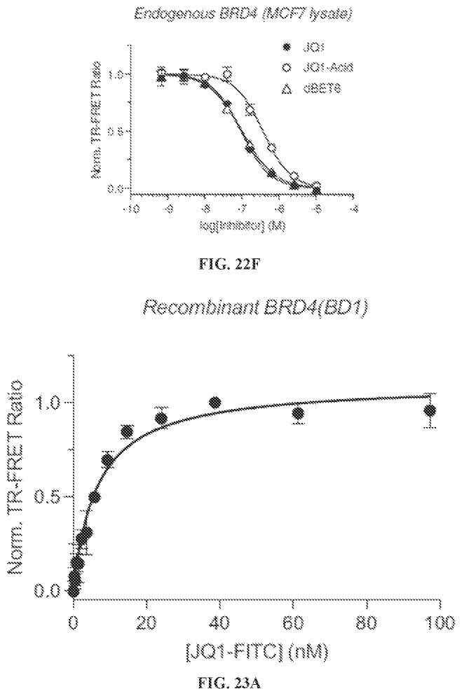

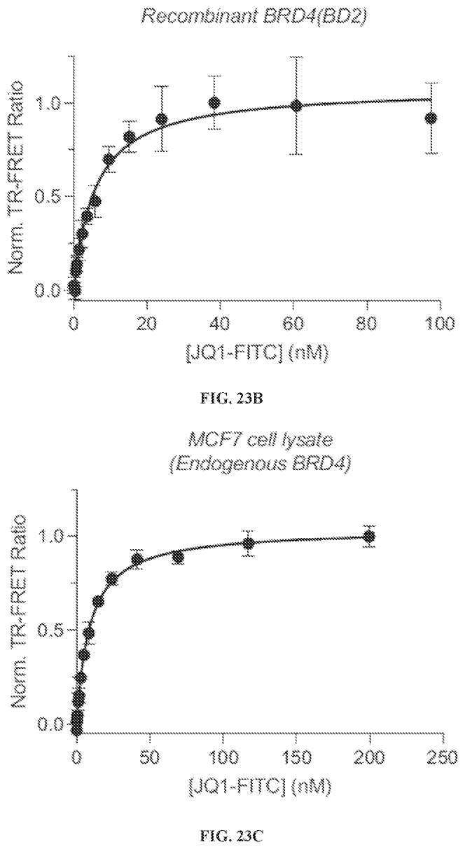

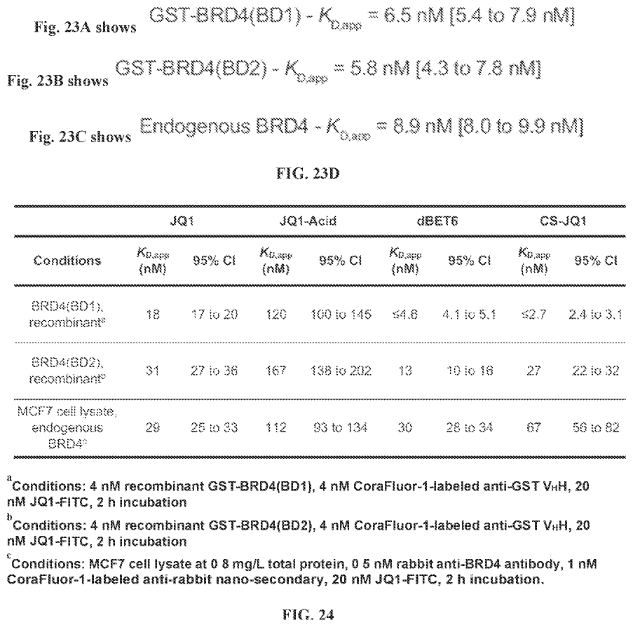

Because of its extensive use in PROTAC development, BRD4 was selected as an exemplary protein of interest for proof-of-concept studies. Based on the potent prototype BRD4 inhibitor JQ1, JQ1-FITC (1, FIG. 22B) was synthesized as a tracer and validated for its applicability with individual recombinant bromodomains BRD4(BD1) and BRD4(BD2). As shown in FIG. 23, the tracer potently bound both isolated bromodomains (K_{D,app}=6.5±1.1 nM and 5.8±1.5 nM for BRD4(BD1) and BRD4(BD2), respectively).

Quantifying BRD4 levels in response to degrader treatment: following validation of the target engagement assay for recombinant proteins, the system was applied for the detection of endogenous BRD4. For ligand displacement assays. the tracer is canonically used at or around its K_{D,app}. In contrast, in protein quantification experiments, the “titration regime” is desired where the tracer concentration is much greater than the K_{D,app} to maximize occupancy. In the preliminary experiments, dBET6, a potent BRD4 degrader, was chosen as a positive control due to its well-established activity.

Titration of various lysate dilutions with fixed concentrations of primary anti-BRD4 IgG (0.5 nM), CoraFluor-1-labeled nano-secondary (I nM), and JQ1-FITC (20 nM) demonstrated linearity over several orders of magnitude with an estimated lower detection limit of ˜10 μg/mL total protein (˜25 cells/μL) (FIG. 21C). Under ligand displacement conditions ([JQ1-FITC]K_{D,app} FIG. 23), the K_{D,app} values of JQ1, JQ1-Acid and dBET6 toward endogenous BRD4 were determined, which closely matched reference data (FIG. 21D-F, Table in FIG. 24).

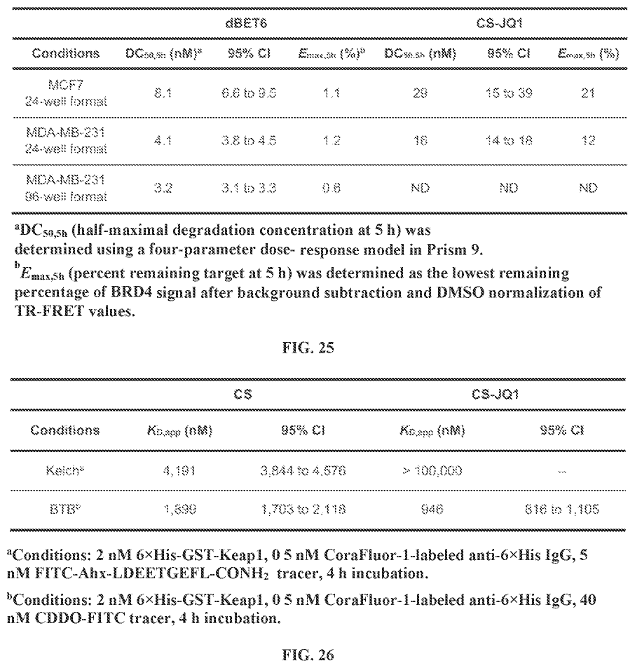

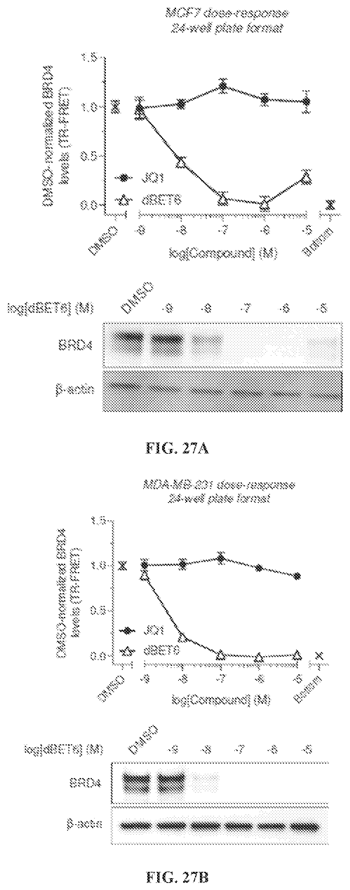

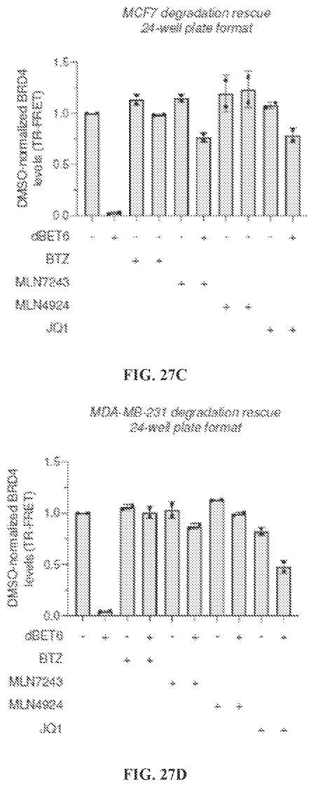

Next, under protein titration conditions ([JQ1-FITC]>>K_{D,app}), dBET6-induced BRD4 degradation was quantified in MCF7 cells. Initially, cells were treated with JQ1 (negative control) or dBET6 (positive control; cₘₐₓ=10 μM) at varying concentrations for 5 h, followed by a 1 h washout to remove excess. Cells were then lysed in mild lysis buffer (see STAR Methods), followed by the addition of the detection mix (100 nM JQ1-FITC (˜11×K_{D,app}), 0.5 nM anti-BRD4 IgG and I nM CoraFluor-1-labeled nano-secondary). Total cell count/protein was achieved by Bradford assay. As expected, a dose-dependent decrease in TR-FRET signal was observed in cells treated with dBET6, but not JQ1, indicating potent degradation of BRD4 (DC_{50,5h}=8.1±1.5 nM; E_{max,5h}=1.1%; FIG. 27A and Table in FIG. 25). The total time from lysis to TR-FRET measurement was ˜1.5 h. Western blot analysis on the same samples, which required approximately 2 days, provided near-identical results (FIG. 27A).

Next, to demonstrate compatibility with other cell lines, identical experiments in MDA-MB-231 cells were performed (FIG. 27B). Again, dBET6 similarly showed potent BRD4 degradation (DC_{50,5h}=4.1±0.3 nM: E_{max,5h}=1.2%; Table in FIG. 25) with good agreement between our CoraFluor TR-FRET platform and Western blot analysis.

The ability of the developed TR-FRET assay was tested to quantify the rescue of dBET6-induced BRD4 degradation by bortezomib (BTZ), MLN7243, MLN4924 (1 μM), and JQ1 (10 μM), which constitute 20S proteasome, E1 ubiquitin-activating enzyme. NEDD8, and competing inhibitors, respectively, in both MCF7 and MDA-MB-231 cells (250 nM dBET6: FIG. 27C-D). In both cell lines, BRD4 degradation was attenuated by all compounds, consistent with previous reports.

Assay miniaturization to 96-well plate format: PROTAC development and characterization demands the combinatorial analysis of multiple variables including incubation time and compound concentration, which are ideally performed with multiple replicates in parallel to ensure consistency. Accordingly. the number of required data points can quickly grow exponentially. Therefore, rapid, scalable and quantitative assays—especially in unmodified cell lines—are highly desirable. The assay platform was therefore miniaturized and adapted to a 96-well plate format, which increases both throughput and compatibility with automated liquid handling equipment. As shown in FIG. 28A, upon treatment of MDA-MB-231 cells (20,000 cells/well) with varying concentrations of dBET6 and JQ1 (cₘₐₓ=1 μM) for 5 h, followed by cell lysis and addition of TR-FRET detection mix (total time to data acquisition=1 h), a robust dose-dependent decrease was observed in cellular BRD4 levels in dBET6-treated wells (DC_{50,5h}=3.2±0.1 nM, R²=0.99; E_{max,5h}=0.6%; Table in FIG. 25), but not those treated with JQ1.

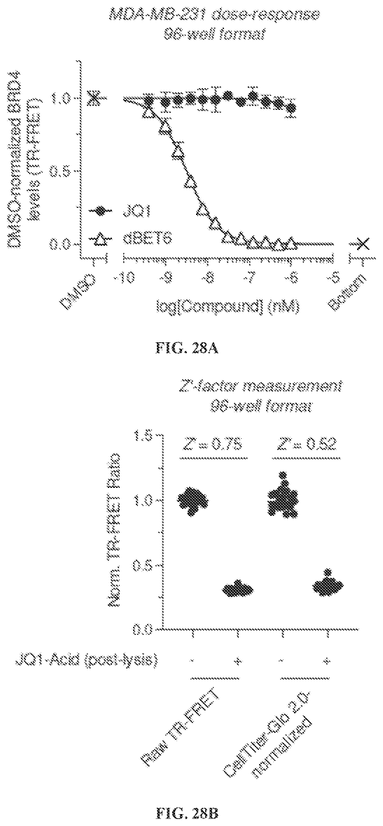

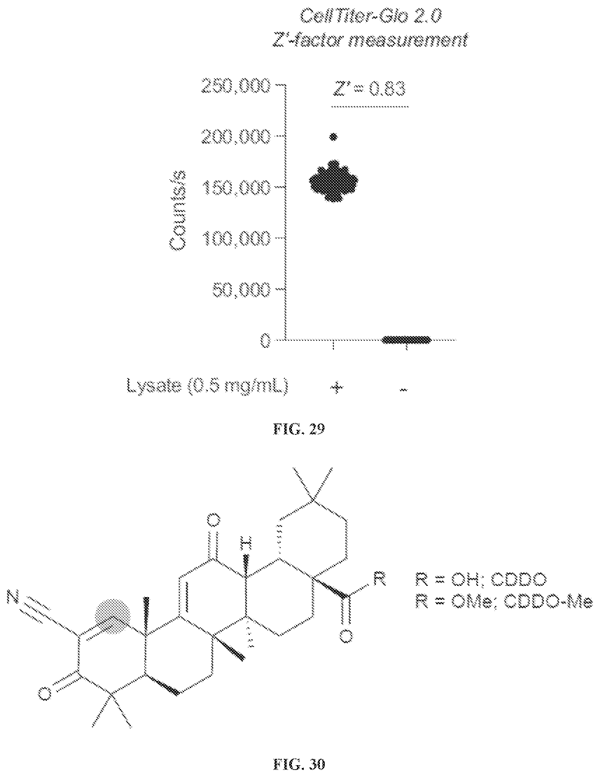

To assess assay performance, the assay robustness was measured which yielded a Z′-factor of 0.75, which is considered excellent for high-throughput screening (HTS) applications (FIG. 28B). Furthermore, to provide an optional mean for data normalization in high-throughput, CellTiter-Glo 2.0 cell viability assay was employed after TR-FRET analysis (FIG. 28B). Since the Z′-factor of CellTiter-Glo 2.0 is 0.83 (FIG. 29), normalization results in an overall reduced Z′-factor of 0.52. Regardless, this is still excellent and well suited for HTS.

Characterization of celastrol-derived BRD4 degrader: recently, the targeting of other E3 ubiquitin ligase complexes, apart from those canonically used such as CRBN and VHL, using (reversible) covalent ligands that target cysteine side chains has gained increasing attention. Of the predicted >600 E3 ligases, a substantial fraction feature solvent-exposed cysteine residues that can potentially be exploited with corresponding sulfhydryl-reactive biasing elements, including the recently targeted RNF114, RNF4, FEM1B, DCAF16, and DCAF11 complexes.

While selective targeting of E3 ligases can potentially provide cell type- or tissue-specific degradation, targeting multiple ligase complexes with a “promiscuous” reversible covalent inhibitor could provide a means for efficient TPD. The triterpenoid celastrol (CS) can form reversible covalent adducts with multiple cysteine nucleophiles and has been shown to bind a host of proteins. CS also targets Keap1 (Kelch-like ECH-associated protein 1), a redox-regulated member of the CRL3 (Cullin-RING E3 ligase) complex that regulates homeostatic abundance of the transcription factor Nrf2 (abbreviation). CS binds Keap1 BTB and Kelch domains with low micromolar affinity (Table in FIG. 26). A previous report has demonstrated the promise of PROTACs (CDDO-JQ1) derived from bardoxolone methyl (CDDO-Me), a synthetic triterpenoid that binds Keap1 with high affinity (FIG. 30). This example shows the capacity of CS to function as a recruiting element for E3 ligase activity in a similar manner.

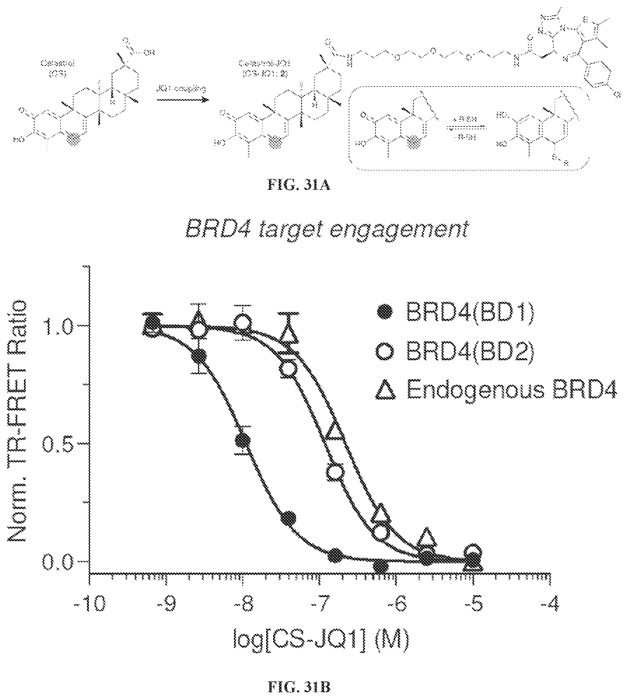

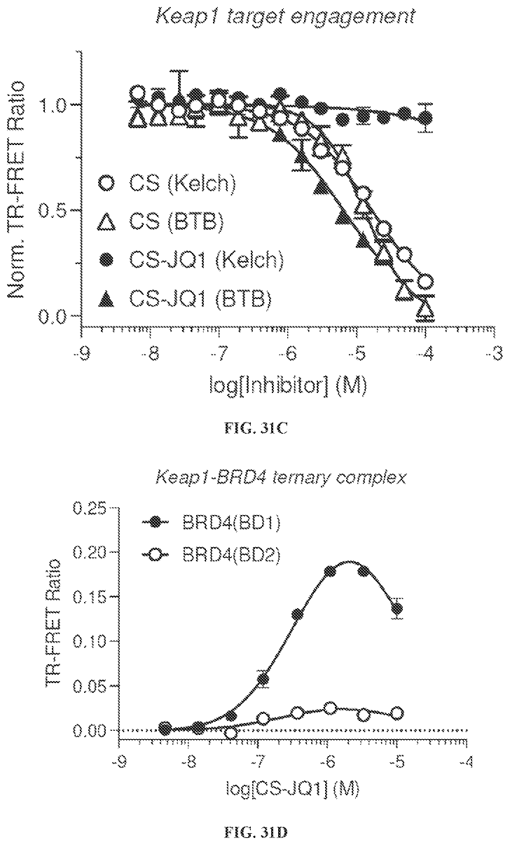

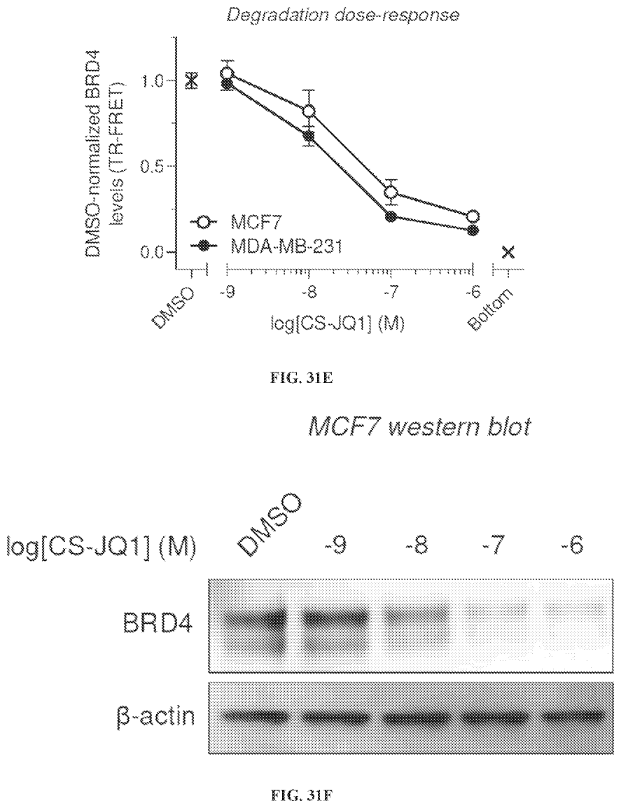

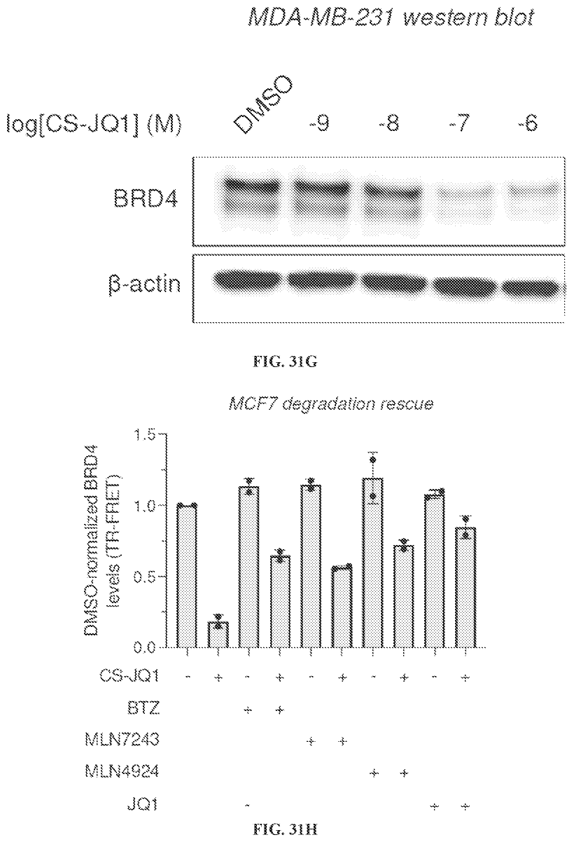

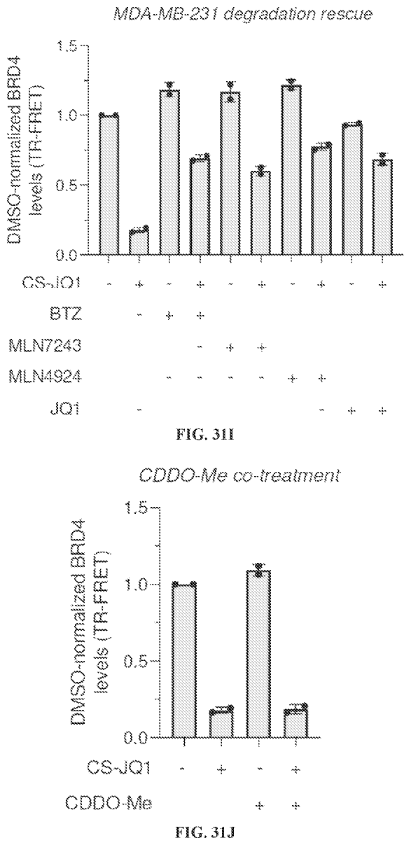

A PROTAC compound CS-JQ1 (2, FIG. 31A) was prepared as described herein. Data shows that CS-conjugation did not impair binding to BRD4 (FIG. 31B and Table in FIG. 24). Surprisingly and unexpectedly, however, CS-JQ1 lost the ability to bind the Keap1 Kelch domain, while exhibiting slightly improved affinity for the BTB domain (FIG. 31C and Table in FIG. 26). In contrast. functionalization of CDDO results in substantially decreased affinity for BTB. Furthermore, CS-JQ1 was able to induce ternary complex formation between wildtype, full-length Keap1, and isolated BRD4(BD1) and BRD4(BD2) domains (FIG. 31D).

Next, MCF7 and MDA-MB-231 cells were treated for 5 h with varying concentrations of CS-JQ1, followed by BRD4 quantification. CS-JQ1 showed dose-dependent and efficient degradation of BRD4 in both cell lines (DC_{50,5h,MCF7}=29±14 nM; E_{max,5h,MCF7}=21%; DC_{50,5h,MDA-MB-231}=16±2 nM; E_{max,5h,MDA-MB-231}=12%; FIG. 31E and Table in FIG. 25). Western blot analysis on the same samples yielded results that were again in excellent agreement (FIG. 31F-G).

Next, 20S proteasomal, E1 ubiquitin-activating enzyme was evaluated, as well as NEDD8-dependence on CS-JQ1 activity by co-treatment with 1 μM of either BTZ, MLN7243, or MLN4924, respectively (FIG. 31H-I). In both cell lines, CS-JQ1-mediated BRD4 degradation (250 nM) was substantially rescued by all three compounds. indicating proteasomal and ubiquitin system dependence. As expected, co-treatment with JQ1 (10 μM) similarly reduced CS-JQ1 degradation efficiency (FIG. 31H-I). However, when the capacity of CDDO-Me was evaluated (CDDO was shown to bind Keap1 BTB domain with potent activity (K_{D,CDDO-Me}=24 nM)) to attenuate CS-JQ1-induced BRD4 degradation similar to JQ1, it was surprisingly and unexpectedly found that CDDO-Me co-treatment did not increase BRD4 levels.

### Discussion of Results

The quantification of cellular protein levels in response to various stimuli represents one of the fundamental experimental techniques employed in chemical biology research. To overcome the inherent limitations of Western blot analysis and ELISA, bioluminescence-based assay formats have been recently developed to simplify workflow, increase throughput, and improve sensitivity. In particular, the use of luciferase tags, such as Promega's HiBiT system. have gained increasing attention. While these approaches can even enable real-time detection of POI levels in living cells, they are not compatible with unmodified endogenous protein but rather require the expression of the POI as a (split-)luciferase fusion protein. This is usually accomplished by transfection with a corresponding expression vector or by genomic editing of a target cell line. The latter constitutes a lengthy process that generally takes weeks to months and needs to be performed for each cell line of interest.

In contrast, the approach validated in this example is directly applicable to unmodified cell lines, demonstrates excellent robustness and low technical variability, and is readily adaptable to high-throughput formats, enabling quantitative measurements within 1 h. Unlike luminescence, TR-FRET does not require addition of a luciferase substrate and enzymatic turnover, which provides long signal stability and superior temporal control over assay readout. Another significant improvement is the adaptation of TR-FRET donor-labeled nano-secondaries, which circumvent the need for conjugation of the TR-FRET donor to individual primary antibodies and should find general acceptance for antibody tagging. Furthermore, the monovalent nature of nanobodies avoids the formation of higher order immune complexes common to the use of multivalent secondary detection reagents (e.g. antibodies and streptavidin), which can cause a nonlinear signal response.

The critical prerequisites for the approach are (i) the availability of a primary antibody that recognizes the native POI and (ii) a linker-modified small-molecule ligand that binds the POI with sufficient potency. Both requirements are generally met early on in PROTAC development, which allows for facile implementation of the methodology. Importantly, this approach provides easy access to a ligand displacement platform for the profiling of POI ligands using endogenous protein as a collateral benefit. Owing to the inherent characteristics of TR-FRET, POI-selectivity of the tracer is not required provided that the employed antibody is selective for the POI and does not recognize other proteins to which the tracer also binds.

### Methods Used in the Example

Mammalian cell culture: MCF7 cells (ATCC) were propagated in RPMI-1640 medium supplemented with 10% FBS, 1% pen-strep, and 1% L-glutamine at 37° C. and 5% CO₂. MDA-MB-231 cells (ATCC) were propagated in DMEM medium supplemented with 10% FBS, and 1% pen-strep at 37° C. and 5% CO₂.

Preparation of MCF7 cell extracts: a cell pellet from one 15 cm dish (˜25 M cells) of MCF7 cells was allowed to thaw on ice and cells were suspended in 400 μL lysis buffer (25 mM HEPES, 150 mM NaCl, 0.2% (v/v) Triton X-100, 0.02% (v/v) TWEEN-20, pH 7.5 supplemented with 2 mM DTT, 250 U Benzonase (Sigma E1014) and 1 mM AEBSF hydrochloride (Combi-Blocks SS-7834)). Optionally, Roche cOmplete, Mini, EDTA-free protease inhibitor cocktail (Sigma 11836170001) can be used in place of, or in combination with, AEBSF hydrochloride. Cells were homogenized via passage through a 27.5-gauge needle 5 times, and the resulting mixture was incubated with slow, end-over-end mixing at 4° C. for 30 min. The lysate was clarified via centrifugation at 16,100×g for 20 min at 4° C. then 800 μL (1:3 dilution) dilution buffer (25 mM HEPES, 150 mM NaCl, 0.005% (v/v) TWEEN-20, pH 7.5) was added and the lysate was re-clarified at 16,100×g for 20 min at 4° C. Total protein was quantified via detergent-compatible Bradford assay (ThermoFisher 23246) The lysate was used fresh or flash-frozen in liquid nitrogen and stored at −80° C. in single-use aliquots.

TR-FRET measurements: unless otherwise noted, experiments were performed in white, 384-well microtiter plates (Corning 3572) in 30 μL assay volume. TR-FRET measurements were acquired on a Tecan SPARK plate reader with SPARKCONTROL software version V2.1 (Tecan Group Ltd.), with the following settings: 340/50 nm excitation, 490/10 nm (Th) and 520/10 nm (FITC) emission, 100 μs delay, 400 μs integration. The 490/10 and 520/10 emission channels were acquired with a 50% mirror and a dichroic 510 mirror, respectively, using independently optimized detector gain settings unless specified otherwise. The TR-FRET ratio was taken as the 520/490 nm intensity ratio on a per-well basis.

Antibody and nanobody labeling: nano-secondary alpaca anti-rabbit IgG (ChromoTek shurbGNHS-1), GST V_{H}H (ChromoTek st-250), anti-6×His IgG (Abcam 18184), and anti-GST IgG (Abcam 19256) were labeled with CoraFluor-1-Pfp as previously described. The following extinction coefficients were used to calculate protein concentration and degree-of-labeling (DOL): ChromoTek shurbGNHS-1 E₂₈₀=24,075 M⁻¹cm⁻¹, ChromoTek st-250 E₂₈₀=28,545 M⁻¹cm⁻¹, IgG E₂₈₀=210,000 M⁻¹cm⁻¹, CoraFluor-1-Pfp E₂₈₀=22,000 M⁻¹cm⁻¹. Nanobody conjugates were diluted with 50% glycerol and stored at −20° C. IgG conjugates were diluted with 50% glycerol, snap-frozen in liquid nitrogen, and stored at −80° C.

Determination of apparent equilibrium dissociation constant (K_{D,app}) of JQ1-FITC toward individual recombinant bromodomains and endogenous BRD4 in MCF7 lysate: recombinant BRD4(BD1) and BRD4(BD2) were purchased from BPS Biosciences, Inc and Epicypher, Inc (GST-BRD4(BD1), 31040; GST-BRD4(BD2), 15-0013, respectively). Recombinant bromodomains were diluted to 0.5 nM in assay buffer (25 mM HEPES, 150 mM NaCl, 0.5 mg/mL BSA, 0.005% TWEEN-20, pH 7.5) with 2 nM CoraFluor-1-labeled anti-GST V_{H}H, then JQ1-FITC was added in serial dilution (1:1.6 titration, 15-point, cₘₐₓ=100 nM) using a D300 digital dispenser (Hewlett-Packard) and allowed to equilibrate for 2 h at room temperature before TR-FRET measurements were taken. Nonspecific signal was determined with 50 μM JQ1-Acid, and data were fitted to a One Site—Specific Binding model in Prism 9.

For the profiling of endogenous BRD4, MCF7 cell lysate as prepared above was diluted to 0.8 mg/mL total protein in 1:3 lysis buffer:dilution buffer with 0.5 nM rabbit anti-BRD4 antibody (Cell Signaling Technology: E2A7X) and 1 nM CoraFluor-1-labeled anti-rabbit nano secondary. JQ1-FITC was added in serial dilution (1:1.7 titration, 15-point, cₘₐₓ=200 nM) using a D300 digital dispenser and allowed to equilibrate for 2 h at room temperature before TR-FRET measurements were taken. Nonspecific signal was determined with 50 μM JQ1-Acid, and data were fitted to a One Site—Specific Binding model in Prism 9.

TR-FRET ligand displacement assays: the following assay parameters have been used: (i) 4 nM GST-BRD4(BD1), 4 nM CoraFluor-1-labeled anti-GST V_{H}H, 20 nM JQ1-FITC in assay buffer, (ii) 4 nM GST-BRD4(BD2), 4 nM CoraFluor-1-labeled anti-GST V_{H}H, 20 nM JQ1-FITC in assay buffer, (iii) MCF7 cell lysate at 0.8 mg/mL total protein, 0.5 nM rabbit anti-BRD4 antibody, 1 nM CoraFluor-1-labeled anti-rabbit nano secondary, 20 nM JQ1-FITC. In all cases, test compounds were added in serial dilution (1:4 titration, 9-point, Cₘₐₓ=10 μM) using a D300 digital dispenser and allowed to equilibrate for 2 h at room temperature before TR-FRET measurements were taken. The assay floor (background) was defined with the 10 M JQ1 dose, and the assay ceiling (top) was defined via a no-inhibitor control. TR-FRET ligand displacement assays with 6×His-GST-Keap1 construct were performed as previously described. Data were background corrected, normalized and fitted to a four-parameter dose response model using Prism 9.

Calculation of inhibitor K_{D,app} values from measured TR-FRET IC₅₀: For TR-FRET ligand displacement assays with recombinant proteins and for endogenous BRD4 in whole cell extract, we have determined the K_{D,app} of the respective fluorescent tracer under each assay condition. Inhibitor K_{D,app} values have been calculated using Cheng-Prusoff principles, outlined in Equation 1 below:

$${K}_{D , app} = \frac{{IC}_{50}}{1 + \frac{[ S ]}{{K}_{X}}} \qquad ( 1 )$$

Where IC₅₀ is the measured IC₅₀ value, [S] is the concentration of fluorescent tracer, and K_{X} is the K_{D,app} of the fluorescent tracer for a given condition (Cheng and Prusoff, 1973).

TR-FRET assay to measure ternary complex formation between full-length, wildtype Keap1 and individual recombinant bromodomains: Keap1 (tag-free; 11981-HNCB; Sino Biological) was diluted to 40 nM in assay buffer (supplemented with 1 mM DTI) containing 40 nM FITC-Ahx-LDEETGEFL-CONH₂ tracer, 20 nM CoraFluor-1-labeled anti-GST antibody, and either 40 nM GST-BRD4(BD1) or GST-BRD4(BD2). CS-JQ1 was added in serial dilution (1:4 titration, 9-point, cₘₐₓ=10 μM) using a D300 digital dispenser and allowed to equilibrate for 4 h at room temperature before TR-FRET measurements were taken with identical detector gain settings. Data were background-subtracted from wells containing no CS-JQ1.

TR-FRET BRD4 quantification assay in 24-well plate format: MCF7 or MDA-MB-231 cells were seeded into 24-well plates (Corning 353047) at a density of 200,000 cells/well in 0.5 mL cell culture medium and allowed to adhere overnight. For dose-response profiling, a D300 digital dispenser was used to dispense serial dilutions of test compounds (1:10 titration, 6-point, cₘₐₓ=1 or 10 μM) normalized to 0.1% DMSO. Cells were incubated for 5 h at 37° C. and 5% CO₂ then media was replaced with pre-warmed cell culture medium (1 mL/well) and residual test compound was washed out for 1 h at 37° C. and 5% CO₂. After, media was aspirated and cells were washed with PBS (2 mL/well), followed by the addition of ice-cold lysis buffer (200 μL/well). The plate was shaken at 200 rpm on an orbital shaker (IKA KS 260 basic) for 10 min, then lysate was transferred to 1.5 mL Eppendorf tubes and further incubated with slow, end-over-end mixing for 10 min at 4° C. The lysate was clarified via centrifugation at 16,100×g for 10 min at 4° C. then total protein concentration was measured using a detergent-compatible Bradford assay (ThermoFisher 23246). Lysate was transferred to a 384-well plate (30 μL×3 TR-FRET replicates) then 5 μL of 7× detection mix (0.5 nM rabbit anti-BRD4 antibody, 1 nM CoraFluor-1-labeled anti-rabbit nano-secondary, 100 nM JQ1-FITC final concentrations, prepared in dilution buffer) was added to each well and allowed to equilibrate for 1 h before TR-FRET measurements were taken. TR-FRET ratios were background-subtracted from wells containing lysis buffer, 0.5 mg/mL BSA, and detection mix, then normalized to total protein concentration. The average TR-FRET intensity was normalized to DMSO for each biological replicate before being analyzed in Prism 9. For degradation rescue experiments, a D300 digital dispenser was used to dispense rescue compounds (see respective figure panels for concentrations) normalized to 0.2% DMSO and were pre-incubated for 30 min at 37° C. and 5% CO₂ before degraders (250 nM) were added. Cell treatment, lysate preparation and TR-FRET analysis was performed as described above.

TR-FRET BRD4 quantification assay in 96-well plate format: MDA-MB-231 cells were seeded into 96-well plates (Corning 3904) at a density of 20,000 cells/well in 100 μL cell culture medium and allowed to adhere overnight. A D300 digital dispenser was used to dispense serial dilutions of test compounds (1:2 titration, 12-point, cₘₐₓ=1 μM) normalized to 0.1% DMSO. Cells were incubated for 5 h at 37° C. and 5% CO₂ then media was replaced with pre-warmed cell culture medium (150 μL/well) and residual test compound was washed out for 1 h at 37° C. and 5% CO₂. After, media was aspirated and cells were washed with PBS (200 μL/well), followed by the addition of ice-cold lysis buffer (60 L/well). The plate was shaken at 1,000 rpm on an orbital shaker (Boekel Scientific Jitterbug, model 130000) for 10 min, then 10 μL of 7× detection mix (0.5 nM rabbit ant-BRD4 antibody, 1 nM CoraFluor-1-labeled anti-rabbit nano-secondary. 100 nM JQ1-FITC final concentrations, prepared in dilution buffer) was added to each well and allowed to equilibrate for 1 h. The plate was centrifuged at 2,000×g for 1 min then lysate was transferred to a 384-well plate (30 μL×2 TR-FRET replicates) using an adjustable electronic multichannel pipette (Matrix Equalizer, ThermoFisher 2231) and TR-FRET measurements were taken. Optionally, after TR-FRET measurements, 5 μL/well of CellTiter-Glo 2.0 reagent (Promega G9241) was added to the wells of the 384-well plate and allowed to equilibrate for 10 minutes before luminescence intensity was recorded on a Tecan SPARK plate reader (luminescence module, no attenuation, 250 ms integration time, output Counts/s). TR-FRET ratios were background-subtracted from wells containing lysis buffer, 0.5 mg/mL BSA, and detection mix. The average TR-FRET intensity was normalized to DMSO for each biological replicate, then data were fitted to a four-parameter dose response model using Prism 9.

Immunoblotting: proteins in lysates (10 μg) were analyzed by electrophoresis on 3-8% SDS-polyacrylamide gels (ThermoFisher) and subsequently transferred to a nitrocellulose membrane (Bio-Rad). All antibodies were purchased from Cell Signaling Technology. Ponceau staining and R-actin probing (8H10D10, 1:1,000) were used to verify equal protein loading on the blot. The membrane was blocked using 5% nonfat milk powder in TBS-T (Tris-buffered saline: 0.1% TWEEN-20) at room temperature for 1 h and then incubated with an anti-rabbit IgG BRD4 antibody (E2A7X, 1:750) in 2.5% nonfat milk overnight at 4 C. The membrane was then incubated with an anti-rabbit IgG HRP-linked antibody (1:5,000 in 2.5% nonfat milk: 7074S). The proteins were detected using SuperSignal™ West Femto Maximum Sensitivity Substrate (ThermoFisher).

Reagents and ligands: reagents and ligands were purchased from Chem-Impex International, Millipore-Sigma, TCI America, Beantown Chemical. Combi-Blocks, MedChemExpress, Ontario Chemicals, and BOC Sciences and used as received.

Chemical synthesis: FITC-Ahx-LDEETGEFL-CONH₂ (FITC-KL9) peptide tracer was custom synthesized by Genscript (Piscataway, New Jersey). CDDO-FITC fluorescent tracer was prepared as previously described. Column purifications were performed on a Biotage Isolera 4 Purification System equipped with a 200-400 nm diode array detector. For normal phase flash purifications, Sorbtech Purity Flash Cartridges were used (CFC-52300-012-18 and CFC-52500-025-12). For reverse phase flash purifications, Biotage Sfar Bio C18 Duo 300 A, 20 μm cartridges were used (FSBD-0411-001). Analytical LC/MS was performed on a Waters 2545 HPLC equipped with a 2998 diode array detector, a 2424 evaporative light scattering detector, a 2475 multichannel fluorescence detector, and a Waters 3100 ESI-MS module, using a XTerraMS C18 5 μm, 4.6×50 mm column at a flow rate of 5 mL/min with a linear gradient (95% A: 5% B to 100% B 90 sec and 30 sec hold at 100% B, solvent A=water+0.1% formic acid, solvent B=acetonitrile+0.1% formic acid). LC/MS data analysis was performed using Waters Masslynx V4.1 SCN 846 software. Proton and carbon nuclear magnetic resonance (¹H and ¹³C NMR spectra) were recorded on a Bruker Avance III 400 spectrometer using Topspin 3.2 software and data were analyzed using MestreNova (version 12.0.1-20560, Mestrelab Research). Chemical shifts for protons are reported in parts per million (ppm) and are referenced to residual solvent peaks. Data is reported as follows: chemical shift, multiplicity (s=singlet, br s, =broad singlet, d=doublet, t=triplet, q=quartet, p=pentet, m=multiplet), proton coupling constants (J, Hz), and integration.

### Preparation of 5-(3-(1-(4-(4-chlorophenyl)-2,3,9-trimethyl-6H-thieno[3,2-f][1,2,4]triazolo[4,3-a][1,4]diazepin-6-yl)-2-oxo-7,10,13-trioxa-3-azahexadecan-16-y)thioureido)-2-(6-hydroxy-3-oxo-3H-xanthen-9-yl)benzoic acid—JQ1-FITC—(1)

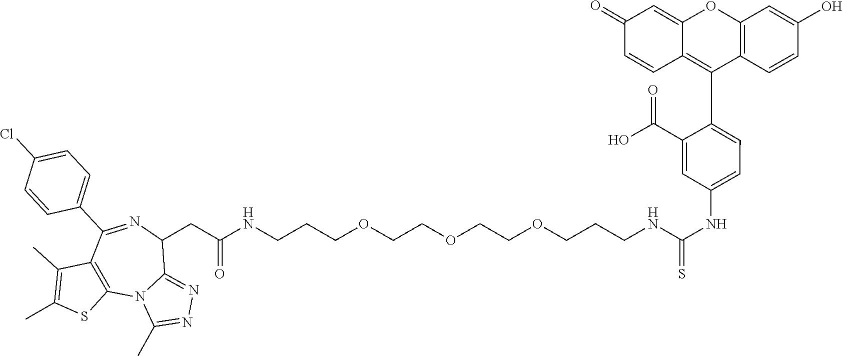 [SMILES: Cc1sc2c(c1C)C(c1ccc(Cl)cc1)=NC(CC(=O)NCCCOCCOCCOCCCNC(=S)Nc1ccc(-c3c4ccc(=O)cc-4oc4cc(O)ccc34)c(C(=O)O)c1)c1nnc(C)n1-2]

1-(4-(4-chlorophenyl)-2,3,9-trimethyl-6H-thieno[3,2-f][1,2,4]triazolo[4,3-a][1,4]diazepin-6-yl)-2-oxo-7,10,13-trioxa-3-azahexadecan-16-aminium chloride (6.0 mg, 9.4 μmol, 1.1 eq) was dissolved in DMF (200 μL) with DIPEA (15 μL, 11 mg, 85 μmol, 10 eq) then 5(6)-fluorescein isothiocyanate (FITC; 3.3 mg, 8.5 μmol, 1 eq) was added and the reaction mixture was briefly vortexed and allowed to stand at room temperature for 10 min. The reaction mixture was directly purified via reverse phase flash chromatography (A 220 nm, 250 nm: gradient: 5% ACN/H₂O+0.1% formic acid for 3 CV, 5% to 70% ACN/H₂O+0.1% formic acid over 14 CV, 70% ACN/H₂O to 100% ACN+0.1% formic acid over 1 CV, 100% ACN+0.1% formic acid for 3 CV). Yield=4.0 mg, 48% as a yellow solid. ¹H NMR (400 MHz, DMSO-d6) o 10.20 (br s, 3H), 8.42-8.32 (m, 1H), 8.30-8.15 (m, 2H), 7.75 (d, J=7.7 Hz, 11H), 7.48 (d, J=8.5 Hz, 2H), 7.41 (d, J=8.5 Hz, 2H), 7.16 (d, J=8.3 Hz, 1H), 6.66 (s, 2H), 6.62-6.52 (m, 4H), 4.50 (t, J=7.1 Hz, 1H), 3.53-3.42 (m, 12H), 3.29-3.08 (m, 6H), 2.59 (s, 3H), 2.40 (s, 3H), 1.83-1.76 (m, 2H), 1.71-1.65 (m, 2H), 1.61 (s, 3H). ¹³C NMR (101 MHz, DMSO-d6) o 169.46, 168.58, 163.04, 159.63, 155.12, 151.93, 149.83, 146.83, 141.50, 136.76, 135.24, 132.27, 130.71, 130.14, 129.84, 129.57, 129.07, 128.49, 125.01, 124.06, 112.68, 109.79, 102.24, 69.78, 69.58, 68.20, 68.05, 53.90, 37.65, 35.79, 29.46, 28.58, 14.08, 12.70, 11.32. MS (ESI^{+/-}) m/z (M+H)⁺ 992.45, m, (M−H)⁻ 990.25, [calculated C₅₀H₅₀ClN₇O₉S₂: 991.28].

### Preparation of(2R,4aS,6aS,12bR,14aS,14bR)-N-(1-(4-(4-chlorophenyl)-2,3,9-trimethyl-6H-thieno[3,2-f][1,2,4]triazolo[4,3-a][1,4]diazepin-6-yl)-2-oxo-7,10,13-trioxa-3-azahexadecan-16-yl)-10-hydroxy-2,4a,6a,9,12b,14a-hexamethyl-11-oxo-1,2,3,4,4a,5,6,6a,11,12b,13,14,14a,14b-tetradecahydropyrene-2-carboxamide—Celastrol-JQ1—(2)

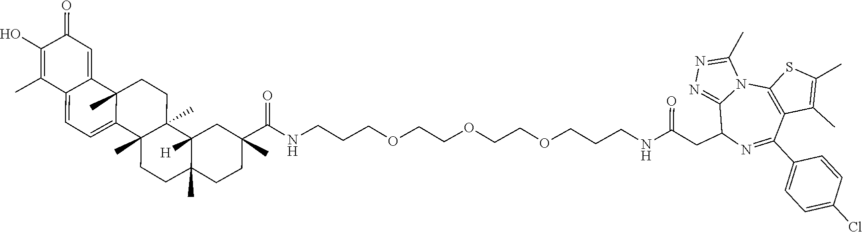

Celastrol (10.0 mg, 22 μmol, 1 eq) was dissolved in DMF (500 μL) then PyBOP (12.1 mg, 23 μmol, 1.05 eq) and 1-(4-(4-chlorophenyl)-2,3,9-trimethyl-6H-thieno[3,2-f][1,2,4]triazolo[4,3-a][1,4]diazepin-6-yl)-2-oxo-7,10,13-trioxa-3-azahexadecan-16-aminium chloride (14.9 mg, 23 μmol, 1.05 eq) were added followed by DIPEA (19 μL, 14 mg, 111 μmol, 5 eq) and the reaction mixture was briefly vortexed then allowed to stand at room temperature for 10 min. The reaction mixture was diluted into EtOAc (50 mL) and the organic layer was washed 2×equal volume 0.2 N HCl, 2×H₂O, 1× saturated brine solution. The organic layer was then dried over Na₂SO₄, filtered, and concentrated. The crude product was purified via reverse phase flash chromatography (A 250 nm, 400 nm: gradient: 5% ACN/H₂O for 3 CV, 5% ACN/H₂O to 100% ACN over 18 CV, 100% ACN for 3 CV). Yield=11.0 mg, 48% as an orange powder. ¹H NMR (400 MHz, CDCl₃) o 7.40 (d, J=8.2 Hz, 2H), 7.32 (d, J=8.3 Hz, 2H), 7.00 (d, J=6.8 Hz, 2H), 6.88 (t, J=5.0 Hz, 1H), 6.63 (t, J=4.4 Hz, 1H), 6.52 (s, 1H), 6.33 (d, J=7.2 Hz, 1H), 4.64 (t, J=7.0 Hz, 1H), 3.68-3.63 (m, 4H), 3.63-3.57 (m, 6H), 3.53 (t, J=5.9 Hz, 3H), 3.45-3.31 (m, 3H), 3.22 (s, 2H), 2.66 (s, 3H), 2.53-2.43 (m, 1H), 2.40 (s, 3H), 2.20 (s, 3H), 2.17 (s, 3H), 2.11-1.75 (m, 10H), 1.67 (br s, 6H), 1.59-1.48 (m, 5H), 1.42 (s, 3H), 1.12 (s, 3H), 1.09 (s, 3H), 0.63 (s, 3H). ¹³C NMR (101 MHz, CDCl₃) o 178.48, 177.66, 170.61, 170.51. 164.94, 163.95, 155.85, 149.99, 146.14, 136.89, 136.82, 134.24, 132.29, 131.06, 130.92, 130.60, 129.98, 128.84, 127.49, 119.67, 118.12, 117.16, 71.34, 70.65, 70.55, 70.37, 69.96, 54.60, 45.22, 44.55, 43.16, 40.22, 39.53, 39.01, 38.30, 37.97, 36.55, 35.17, 33.92, 33.68, 31.78, 31.23, 31.10, 30.96, 30.12, 29.83, 29.45, 29.18, 28.85, 28.71, 21.88, 18.38, 14.56, 13.25, 12.00, 10.42. MS (ESI^{+/-}) m/z (M+H)⁺ 1035.79. m/z (M−H)⁻ 1033.47, [calculated C₅₈H₇₅ClN₆O₇S: 1034.51].

Statistics and reproducibility: biological replicates have been defined as independent cell treatments, performed at different times with biologically distinct samples. For biological replicate analysis, TR-FRET technical replicates refer to the number of replicates performed during the analysis of a given biological sample. For biochemical-based ligand displacement assays, technical replicates refer to the number of parallel replicates used to calculate mean±SD for a given data point within an experiment. No statistical methods were used to predetermine sample size and investigators were not blinded to outcome assessment. Whilst the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references, scientific articles, patent publications, and any other documents cited herein are hereby incorporated by reference for the substance of their disclosure.

### Example 6—Keap 1 Ligand Assays

### Methods

Photophysical characterization: UV-VIS absorption, fluorescence emission and quantum yield measurements were performed on a Horiba DualFL spectrophotometer (Horiba Instruments, Kyoto, Japan) using 1 cm pathlength quartz cuvettes. For quantum yield measurements, the total quantum yield (Φₜₒₜ) values were measured using 12 in 50 mM HEPES buffer, pH 7.4 as the reference (Φ_{tot,ref}=0.50), which was previously determined relative to quinine sulfate in 1.0 N H₂SO₄ (Φᵣ=0.546). Specifically, five separate dilutions of the respective terbium complexes in 50 mM HEPES, pH 7.4 were prepared within the optically dilute limit (OD₃₄₀ ranging from ˜0.25 to 0.04). The OD₃₄₀ and fluorescence emission spectra (450-700 nm, λₑₓ=340 nm) were recorded for all complexes and their respective serial dilutions with identical instrument parameters. Plots of integrated fluorescence intensity (450-700 nm) versus OD₃₄₀ for each dilution series were generated in Prism 8 (GraphPad Software, San Diego, CA) and the slope of the resulting linear regression analysis was solved for Φₜₒₜ in Equation 1 below:

$${ϕ}_{tot} = {∅}_{tot , ref} \times \frac{slope}{{slope}_{ref}} \times {( \frac{η}{{η}_{ref}} )}^{2} \qquad ( 1 )$$

where Φ_{tot,ref}=0.50 (12) and η=η_{ref} are identical refractive indices of water.

Lifetime measurements in either 50 mM HEPES, pH 7.4 or D₂O were performed using a custom photometer setup. Briefly, samples were excited using a mounted 365 nm LED (M365LP1, Thorlabs Inc., Newton, NJ) that was coupled to a cuvette holder (CVH100, Thorlabs Inc.) via an adjustable collimation adapter (ACP2520-A, Thorlabs Inc.). The mounted LED was powered by a pulse modulated LED driver (DC2100, Thorlabs Inc.). Luminescence intensity was detected using an orthogonally mounted amplified high-speed, switchable-gain silicon detector (PDA36A, Thorlabs Inc.) equipped with a long-path filter (FGL610, Thorlabs Inc.) and coupled to a combined oscilloscope and waveform generator (Analog Discovery 2, Digilent Inc., Pullman, WA), which was used to both record the PDA signal and control the LED driver. Data acquisition was performed at a frequency of 1 Hz and 2% duty cycle for n=50 measurements using 1 cm pathlength quartz cuvettes. Individual measurements were combined and averaged in Matlab R2020b (MathWorks, Natick, MA) and further processed in Prism 8 (GraphPad Software). To calculate the lifetime, the negative inverse of the slope of the plot of ln(intensity) versus time in ms was taken.

q and q′_{corr}, which estimate the number of bound water molecules to the metal center of the terbium complexes, were calculated using the lifetime values of each complex in either water (50 mM HEPES, pH 7.4) or D₂O. The calculation of q, which does not correct for the effect of closely diffusing OH oscillators, was achieved via Equation 2:

$$q = A ( \frac{1}{{τ}_{{H}_{2} O}} - \frac{1}{{τ}_{{D}_{2} O}} ) = A \times Δ k \qquad ( 2 )$$

where A=4.2 ms is the proportionality constant for terbium and denotes its sensitivity to quenching by OH oscillators, τ_{H2O} and τ_{D2O} are the lifetimes of the complexes in 50 mM HEPES, pH 7.4 (H₂O) and D₂O, respectively, and Δk is the difference of the radiative rate constants in H₂O and D₂O. The calculation of q′_{corr}, which corrects for the effect of closely diffusing OH oscillators, was achieved via Equation 3:

$${q}_{corr}^{′} = {A}^{′} \times ( Δ k - 0.06 {ms}^{- 1} ) = {A}^{′} \times Δ {k}_{corr} \qquad ( 3 )$$

where A′=5 ms is the proportionality constant for terbium and Δk_{corr} is the corrected value for Δk that takes the effect of closely diffusing OH oscillators into account.

Antibody and nanobody labeling: a 100 μL aliquot of respective IgG antibody (anti-6×His; 18184, Abcam; RT0266, BioXCell) or nanobody (ChromoTek anti-Halo V_{H}H OT-250; HaloTrap) at a concentration of ≥1 mg/mL was buffer exchanged into reaction buffer (100 mM sodium carbonate buffer, pH 8.5+0.05% (v/v) TWEEN-20) using a 0.5 mL, 7K MWCO Zeba-Spin Desalting Column (ThermoFisher 89882) according to the manufacturer's protocol. After buffer exchange into reaction buffer, the appropriate volume of Cora-1-Pfp (2.5 mM in dimethylacetamide, DMAc) was added to achieve a molar ratio of approximately 12-15×Cora-1-Pfp to antibody or 4-5× to nanobody (final DMAc content <5%). The reaction mixture was briefly vortexed and allowed to stand at room temperature for 1 h. Organic solvent and unreacted Pfp ester complex was removed by buffer exchange into storage buffer (50 mM sodium phosphate buffer, pH 7.4, with 150 mM NaCl and 0.05% (v/v) TWEEN-20) using a 0.5 mL, 7K MWCO Zeba™ Spin Desalting Column according to the manufacturer's protocol. The corrected A₂₈₀ value (A_{280,corr}) of antibody/nanobody conjugate was determined via Nanodrop (ND-1000; ThermoFisher: 0.1 cm path length) by measuring A₂₈₀ and A₃₄₀, using Equation 4:

$${A}_{280 , corr} = {A}_{280} - ( {A}_{340} \times cf ) \qquad ( 4 )$$

where cf is the correction factor for the terbium complex contribution to A₂₈₀ and is equal to 0.157. The concentration of antibody/nanobody conjugate, c_{ab/vhh} (M) was determined using Equation 5:

$${c}_{ab / vhh} = \frac{{A}_{280 , corr}}{{ε}_{ab / vhh} \times b} \qquad ( 5 )$$

where ε_{ab} is the antibody extinction coefficient at A₂₈₀, equal to 210,000 M⁻¹cm⁻¹ for standard IgG classes, εᵥₕₕ is the nanobody extinction coefficient (HaloTrap) at A₂₈₀. equal to 23,045 M⁻¹ cm⁻¹, and b is path length in cm (0.1 cm). The concentration of terbium complex, c_{Tb} (M) covalently bound was determined using Equation 6:

$${c}_{Tb} = \frac{{A}_{340}}{{ε}_{Tb} \times b} \qquad ( 6 )$$

where ε_{Tb} is the complex extinction coefficient at A340, equal to 22,000 M⁻¹cm⁻¹ and b is path length in cm (0.1 cm). The degree of labeling (DOL) was calculated using Equation 7:

$$DOL = \frac{{c}_{Tb}}{{c}_{ab / vhh}} \qquad ( 7 )$$

The antibody/nanobody conjugates were diluted with 50% glycerol. Aliquots were snap-frozen in liquid nitrogen and stored at −80° C.

HaloTrap nanobody was also labeled with AF488-Tfp ester (ThermoFisher A37570) using the same methodology, using a correction factor (A₂₈₀/A₄₉₅) of 0.11 and an extinction coefficient of 71,000 (A495) for AF488.

Keap1 tracer characterization: saturation binding curves to determine K_{d,app} values for FITC-KL9 and CDDO-FITC against epitope-tagged Keap1 (His/GST; 11981-H20B; Sino Biological Inc.) were performed with 1 nM Keap1 (His/GST) and 0.5 nM Th-Anti-6×His (Abcam, 18184) in Keap1 assay buffer (25 mM HEPES, 150 mM NaCl, 1 mM DTT, 0.5 mg/mL BSA, 0.005% (v/v) TWEEN-20, pH 7.4). Dose-titration of tracers was performed using a D300 digital dispenser. Titration ranges of 0-31 nM (1:2, 13-point) and 0-125 nM (1:2, 15-point) were used for FITC-KL9 and CDDO-FITC, and nonspecific signal was determined with 25 μM Ac-KL9 or CDDO, respectively. For Cora-1-KL9, K_{d,app} values were determined using 1 nM Keap1 (His/GST) and 0.5 nM AF488-Anti-6×His in Keap1 assay buffer with a dose-titration range of 0-31 nM Cora-1-KL9 (1:2, 13-point). Nonspecific signal was determined with 25 μM Ac-KL9.

Saturation binding curves to determine Kd values for FITC-KL9/Cora-1-KL9 mixture and CDDO-FITC against wildtype Keap1 (tag-free; 11981-HCNB; Sino Biological Inc.) were performed with 1 nM or 4 nM Keap1 (tag-free) in Keap1 assay buffer, respectively, with no additional donor/acceptor present for FITC-KL9/Cora-1-KL9 mix and with 5 nM Cora-1-KL9 present for CDDO-FITC. Dose-titration ranges were 0 to 31 nM (1:1.5 titration, 13-point, total peptide concentration) and 0 to 500 nM (1:1.5 titration, 15-point) for FITC-KL9/Cora-1-KL9 mix and CDDO-FITC, and nonspecific signal was determined with 25 μM Ac-KL9 or CDDO, respectively.

For all experiments, plates (Corning 3572, 30 μL assay volume, triplicate measurements) were incubated for 4 h at room temperature. TR-FRET measurements were acquired on a Tecan SPARK plate reader: 340/50 nm excitation, 490/10 nm (Tb) and 520/10 nm (FITC) emission, 100 μs delay, 400 μs integration. The TR-FRET ratio was taken as the 520/490 nm intensity ratio. In cases where terbium concentration was dynamic, 490 nm emission was normalized to the dispensed concentration of terbium before the TR-FRET ratio was calculated. Data were fitted to a One Site—Specific Binding model using Prism 8 for all experiments except the FITC-KL9/Cora-1-KL9 mixture, in which case a four-parameter nonlinear regression fit model was used.

Measurement of equilibrium dissociation constant of Kean 1 homodimer interaction (K_{d,dimer}): FITC- and Cora-1-KL9 were diluted to 300 nM each (600 nM total tracer concentration) into Keap1 assay buffer in white 384-well plates (Corning 3572, 25 μL assay volume, quadruplicate measurements). Keap1 (tag-free: 11981-HCNB; Sino Biological Inc.) was added in serial dilution from 0 to 500 nM (1:1.4 titration, 7-point) using a D300 digital dispenser and allowed to equilibrate for 2 h at room temperature. TR-FRET measurements were acquired on a Tecan SPARK plate reader: 340/50 nm excitation, 490/10 nm (Tb) and 520/10 nm (FITC) emission, 100 μs delay, 400 μs integration. The TR-FRET ratio was taken as the 520/490 nm intensity ratio. Data were background subtracted, normalized to the concentration of dispensed protein, and log-transformed. The value of K_{d,dimer} was solved via linear regression extrapolation using Prism 8.

Measurement of FITC-KL9 and CDDO-FITC tracer off-rates (k_{off}): Keap1 (His/GST; 11981-H20B; Sino Biological Inc.) was diluted to 1 nM into Keap1 assay buffer with 0.5 nM Tb-Anti-6×His (Abeam, 18184) and either 10 nM FITC-KL9 (6.3×K_{d,app}) or 30 nM CDDO-FITC (4.5×K_{d,app}) in white 384-well plates (Corning 3572, 30 μL assay volume, triplicate measurements). The assay plate was allowed to equilibrate at room temperature for 4 h then an initial (t=0) TR-FRET measurement was taken as described above. Following addition of 40 μM Ac-KL9 and 40 μM CDDO to wells containing FITC-KL9 and CDDO-FITC, respectively, the time-dependent change of TR-FRET intensity was recorded (in 10 s intervals) over the course of 85 min. Data were normalized and fitted to a one-phase decay model using Prism 8.

Measurement of Keap dimer off-rate (k_{off,dimer}): Keap1 (tag-free; 11981-HCNB; Sino Biological Inc.) was diluted to 150 nM into Keap1 assay buffer containing 100 nM each of FITC- and Cora-1-KL9 (pre-mixed solution of peptides; 200 nM total tracer concentration). The solution was allowed to equilibrate at room temperature for 2 h then an initial (t=0) TR-FRET measurement was taken as described above. Following rapid dilution (1:20; Corning 3572) into buffer containing isomolar concentrations of peptide tracer mix, the time-dependent change of TR-FRET intensity was recorded (in 5 s intervals) over the course of 30 min. Data were normalized and fitted to a one-phase decay model using Prism 8.

Inhibitor dose responses with full length Keap1 (tag-free) and FITC/Cora-1-KL9 assay system (homo-dimerization of Keap1; Assay-1): Keap1 (tag-free; 11981-HCNB; Sino Biological Inc.) was diluted to 5 nM into Keap1 assay buffer containing 3.5 nM Cora-1-KL9 and 3.5 nM FITC-KL9 (pre-mixed solution of peptide tracers) in white 384-well plates (Corning 3572, 30 μL assay volume, triplicate measurements). Test compounds were added in serial dilution (1:2 titration, 15-point, cₘₐₓ=10 μM KI-696/Ac-KL9 and 100 μM N-KL9/CDDO/CDDO-Me/CDDO-JQ1/OBT/celastrol/dimethyl fumarate) using a D300 digital dispenser and allowed to equilibrate for 4 h at room temperature. TR-FRET measurements were acquired on a Tecan SPARK plate reader: 340/50 nm excitation, 490/10 nm (Tb) and 520/10 nm (FITC) emission, 100 μs delay, 400 μs integration. The TR-FRET ratio was taken as the 520/490 nm intensity ratio. The assay floor (background) was defined with the 10 μM KI-696 dose, and the assay ceiling (top) was defined via a no-inhibitor control. Data were background corrected, normalized and fitted to a four-parameter dose response model using Prism 8.

Inhibitor dose responses with full length Keap1 (tag-free) construct and Cora-1-KL9/CDDO-FITC assay system (Assay-2): Keap1 (tag-free; 11981-HCNB; Sino Biological Inc.) was diluted to 5 nM into Keap1 assay buffer containing 5 nM Cora-1-KL9 and 150 nM CDDO-FITC in white 384-well plates (Corning 3572, 30 μL assay volume, triplicate measurements). Test compounds were added in serial dilution (1:2 titration, 15-point, cₘₐₓ=10 μM KI-696/Ac-KL9/CDDO/CDDO-Me and 100 μM N-KL9/CDDO-JQ1/OBT/Celastrol/dimethyl fumarate) using a D300 digital dispenser and allowed to equilibrate for 4 h at room temperature. TR-FRET measurements were acquired on a Tecan SPARK plate reader: 340/50 nm excitation, 490/10 nm (Tb) and 520/10 nm (FITC) emission, 100 μs delay, 400 μs integration. The TR-FRET ratio was taken as the 520/490 nm intensity ratio. The assay floor (background) was defined with the 10 μM CDDO dose, and the assay ceiling (top) was defined via a no-inhibitor control. Data were background corrected, normalized and fitted to a four-parameter dose response model using Prism 8.

Plasmid propagation and production: plasmids were transformed into chemically competent DH5α (Fisher FEREC0111) according to manufacturer's protocol. A single transformed colony from a Luria-Bertani (LB)-Ampicillin agar plate was used to inoculate 10 mL of LB Broth (MilliporeSigma 71-753-5) containing Ampicillin (0.1 mg/mL) and the culture was incubated at 37° C. overnight at 225 rpm. The following day, I mL of starter culture was used to inoculate 250 mL LB Broth containing Ampicillin (0.1 mg/mL). which was incubated at 37° C. with shaking at 225 rpm for 16 h. Cells were harvested by centrifugation at 3,000×g for 20 minutes at 4° C. and washed once with Dulbecco's PBS (DPBS). Cell pellets were snap-frozen in liquid nitrogen and stored at −80° C. until plasmid isolation performed.

Transfection-quality plasmid preparations were performed using Qiagen HiSpeed Plasmid Maxi kits (Qiagen 12662) according to manufacturer's protocol. Plasmid DNA was concentrated to >500 ng/μL for mammalian transfection.

Mammalian cell culture: HEK293T cells (ATCC) were propagated in DMEM medium supplemented with 10% FBS, and 1% pen-strep at 37° C. and 5% CO₂.

Preparation of PEI-MAX transfection reagent: PEI-MAX (Polysciences 24765-1) was dissolved in water to a concentration of 1 mg/mL. The pH of the solution was neutralized to pH 7 with NaOH, then sterile filtered (0.22 μm), aliquoted, and stored at −20° C. until further use.

Mammalian protein expression: HDAC1-HaloTag (HDAC1-HT): plasmid pFC14A-HDAC1-HaloTag was custom cloned by Genscript (Piscataway, New Jersey). HEK293T cells were seeded into 15 cm dishes (˜8-10 million cells) to reach ˜70-80% confluency one day prior to transfection. Separately, stock solutions of plasmid DNA (pFC14A-HDAC1-HaloTag: 16 μg/mL) and PEI-MAX (48 μg/mL) were prepared in PBS such that the final volume of each stock solution was 1:20 of the volume of culture media (1:3 w/w DNA:PEI-MAX). The solutions were thoroughly mixed, then the DNA solution was added slowly to the PEI solution and the resulting transfection cocktail (1:10 volume of culture media) was incubated for 20 min at room temperature. The transfection cocktail was added dropwise to the cells (final concentrations: 0.8 μg/mL DNA, 2.4 μg/mL PEI-MAX) and cells were grown for 48 h at 37° C. and 5% CO: (fresh media provided to cells 24 h post-transfection). Cells were harvested via trypsinization, washed twice with PBS, and cell pellets snap-frozen in liquid nitrogen and stored at −80° C. until further use.

Lysis. Cora-1-Halo labeling. and quantification of HDAC1-HT in HEK293T lysate: A cell pellet from one 15 cm dish (˜25M cells) of pFC14A-HDAC1-HT transfected HEK293T cells was allowed to thaw on ice and cells were suspended in 400 μL lysis buffer (50 mM Tris, 150 mM NaCl, 2 mM DTT, 1% (v/v) Triton X-100, 0.1% (w/v) sodium deoxycholate, pH 7.5 supplemented with 250 U Benzonase (Sigma E1014) and 1× protease inhibitor cocktail (Promega G6521)). Cells were homogenized via passage through a 27.5-gauge needle 5 times, and the resulting mixture was incubated with slow, end-over-end mixing at 4° C. for 30 min. The lysate was clarified via centrifugation at 16,100×g for 20 min at 4° C. then 800 μL (1:3 dilution) lx TBS (50 mM Tris, 150 mM NaCl, pH 7.5) was added and the lysate was re-clarified at 16,100×g for 20 min at 4° C.

The resulting diluted, clarified lysate was incubated with 10 μM Cora-1-Halo for 16 h at 4° C. with slow, end-over-end mixing. The labeled lysate was then gel filtrated through a PD-10 desalting column (GE) with exchange buffer (1×TBS+1 mM DTT+0.005% (v/v) TWEEN-20, pH 7.5) to remove excess Cora-1-Halo. PD-10 fractions were tested for protein concentration (Bradford assay, ThermoFisher 23246) and terbium fluorescence (Tecan SPARK plate reader: 340/50 nm excitation, 548/10 nm emission, 100 μs delay, 400 μs integration). Fractions containing both significant protein and terbium fluorescence were pooled. and total protein concentration was determined via Bradford assay.

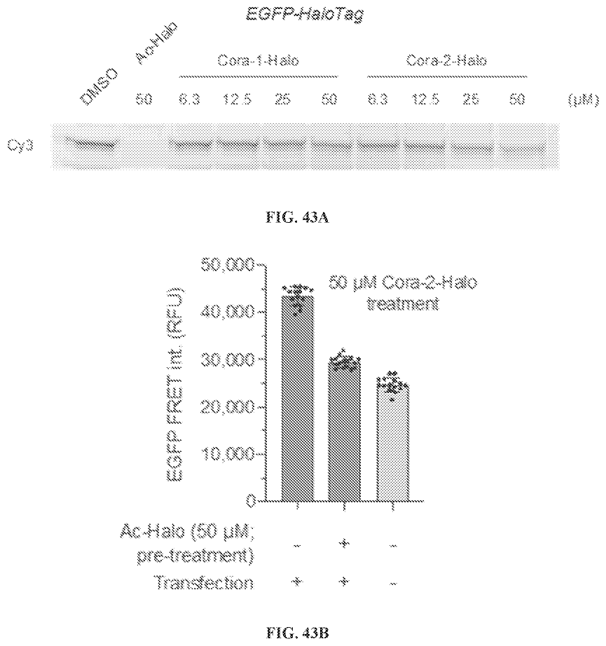

Because HaloTag labeling is stoichiometric (1:1 Cora-1-Halo:HDAC1-HT), the concentration of the Cora-1-Halo labeled HDAC1-HT protein in the pooled, gel-filtrated lysate can be determined via a calibration curve of Cora-1-Halo (0-230 nM, nM increments, 23-step; see FIG. 43). In our experience, the yield of HDAC1-HT from a single 15 cm dish of transfected HEK293T cells was 25-50 μg, resulting in protein concentrations in the pooled, desalted lysate between 275 nM and 550 nM (HDAC1-HT MW=90.6 kDa). Pooled, desalted lysate was diluted 1:5 to remain within the standard curve for quantification, and HDAC1-HT concentration was back-calculated. The labeled lysate was aliquoted, flash frozen in liquid nitrogen, and stored at −80° C. until further use.

We also further quantified the concentration of Cora-1-Halo labeled HDAC1-HT in the lysate via titration with AF488-HaloTrap (FIG. 43). For these experiments, labeled, desalted lysate was diluted 1:12 (˜20-40 nM in HDAC1-HT, 275 μg/mL total protein) and dispensed into white 384-well plates (Corning 3572; 30 μL assay volume, duplicate measurements). AF488-HaloTrap was added in serial dilution from 0 to 150 nM (1:2 titration, 15-point) using a D300 digital dispenser allowed to equilibrate for 24 h at 4° C. TR-FRET measurements were acquired on a Tecan SPARK plate reader: 340/50 nm excitation, 490/10 nm (Tb) and 520/10 nm (AF488) emission, 100 μs delay, 400 μs integration. The TR-FRET ratio was taken as the 520/490 nm intensity ratio. Data were fit according to Equation 8 below, solving for [HaloTag] (HDAC1-HaloTag in this case):

$$Y = 100 \times \frac{( {K}_{d} + [ HaloTrap ] + [ HaloTag ] - \sqrt{{( {K}_{d} + [ HaloTrap ] + [ HaloTag ] )}^{2} - 4 \times [ HaloTrap ] \times [ HaloTag ]}}{( 2 \times [ HaloTag ] )} \qquad ( 8 )$$

Where Y are the observed, normalized TR-FRET ratios, Kd is the equilibrium binding constant for HaloTrap (4 nM), and [HaloTrap] is the concentration of AF488-HaloTrap.

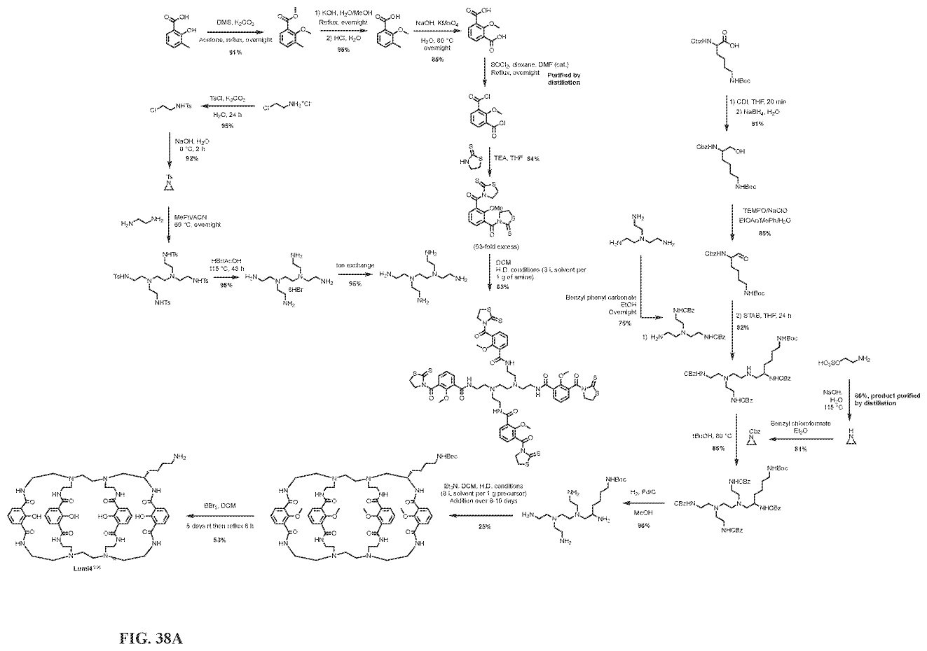

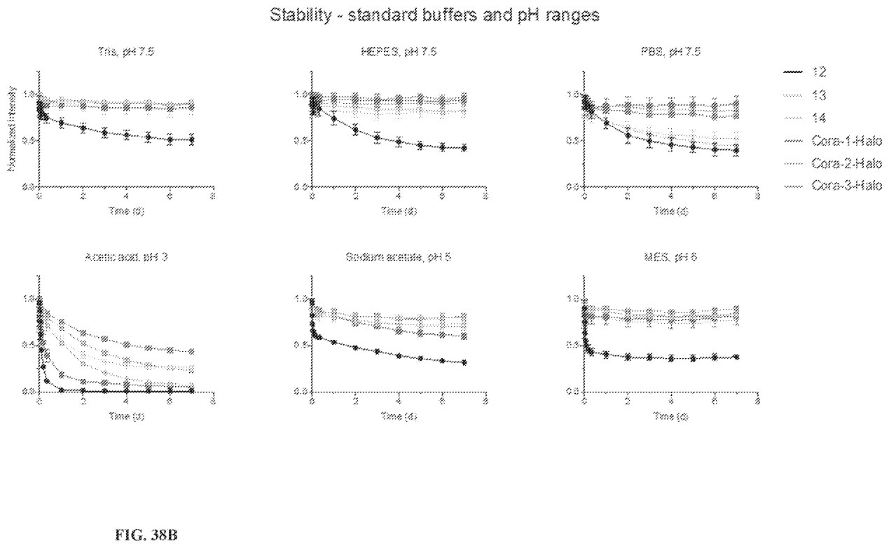

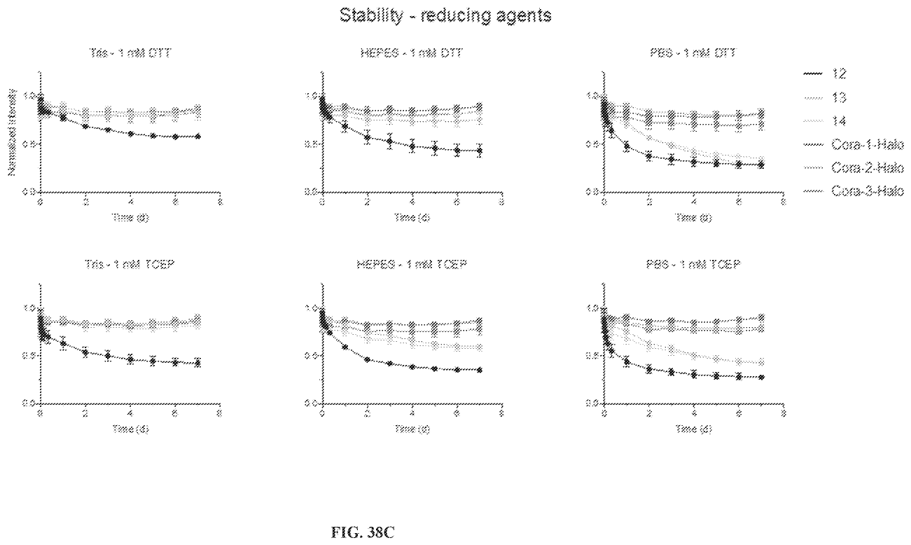

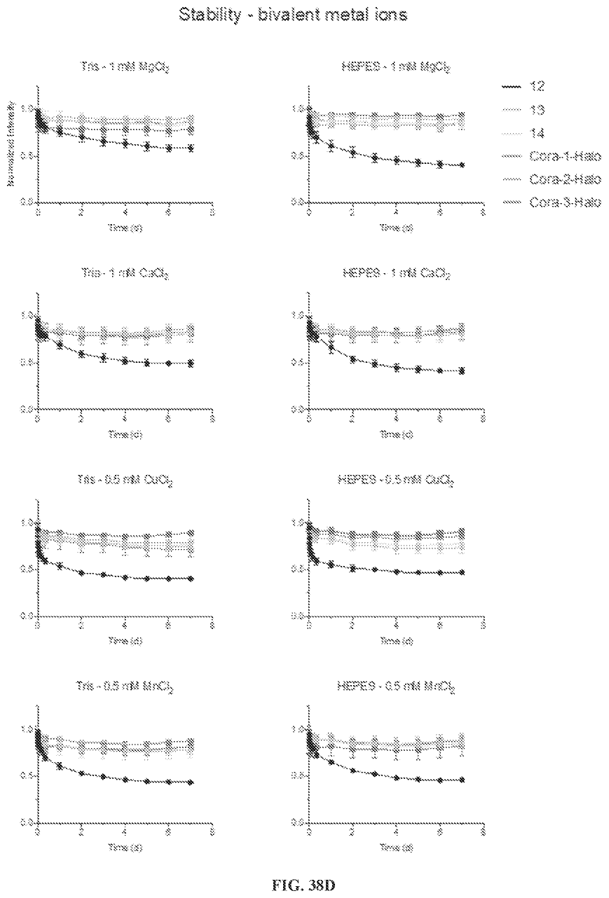

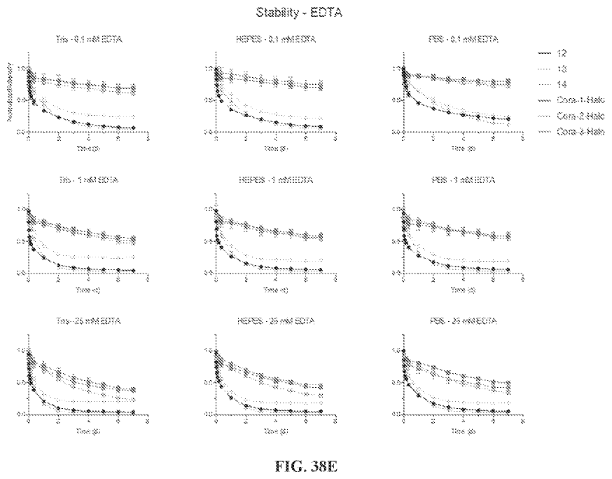

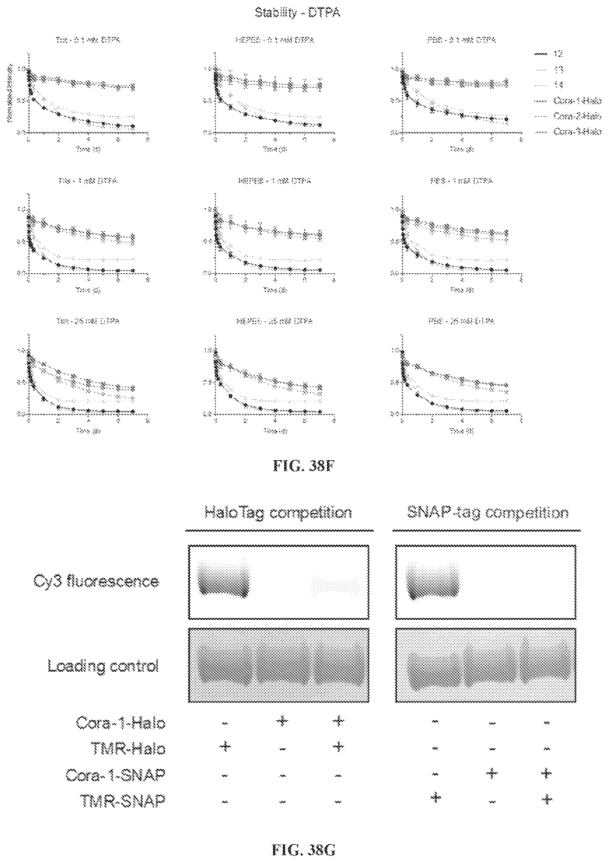

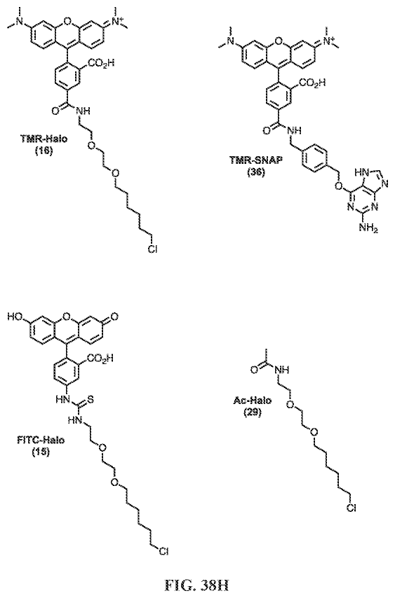

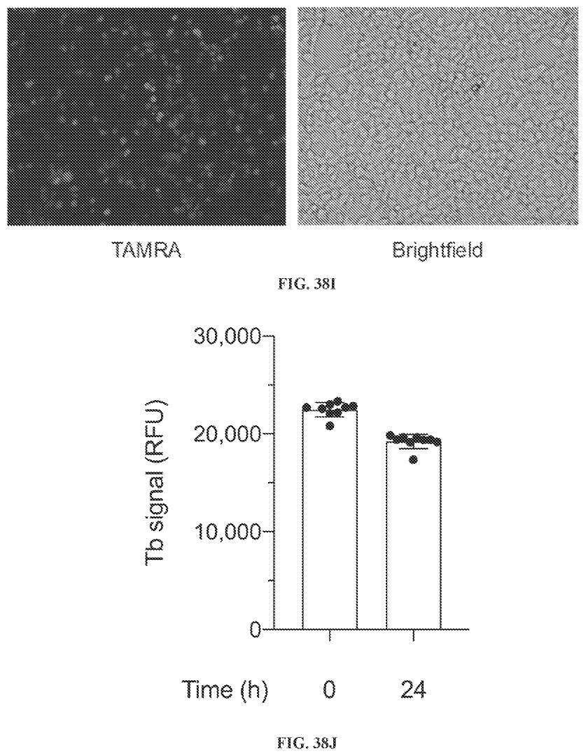

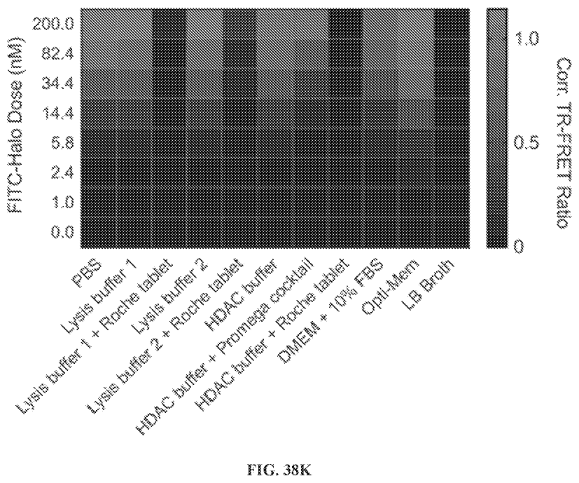

Note: We noticed inefficient HaloTag labeling when using Roche cOmplete™, Mini, EDTA-free Protease Inhibitor Cocktail tablets during the course of our experiments. As a result, we profiled HaloTag labeling efficiency over a wide range of buffer and additive conditions (see FIG. 38K). We observed significant HaloTag inhibition from Roche protease inhibitor tablets, as well as an unknown component(s) of LB-Miller broth. However, protease inhibitor cocktail from Promega (G6521) did not significantly inhibit HaloTag activity and was therefore chosen as the product of choice.

Inhibitor dose responses using HDAC1-HT (Cora-1-Halo labeled), and SAHA-NCT/M344-FITC assay systems in cell lysate: Cora-1-Halo labeled HDAC1-HT cell lysate was diluted to 1 nM in HDAC1-HT with either 12.5 nM SAHA-NCT or 75 nM M344-FITC in HDAC assay buffer (50 mM HEPES, 100 mM KCl, 0.5 mg/mL BSA, 0.001% (v/v) Tween-20, pH 7.5) in white 384-well plates (Corning 3572, 30 μL assay volume, triplicate measurements). The total protein concentration was 15 μg/mL. Test compounds were added in serial dilution (1:2 titration, 15-point, cₘₐₓ=10 μM SAHA/Cpd-60, 1 μM panobinostat, and 100 μM CI-994) using a D300 digital dispenser and allowed to equilibrate for 3 h at room temperature. TR-FRET measurements were acquired on a Tecan SPARK plate reader: 340/50 nm excitation, 490/10 nm (Tb) and 520/10 nm (FITC) for M344-FITC emission, 548 nm (Tb) and 640 nm (NCT) for SAHA-NCT emission, 100 μs delay, 400 μs integration. The TR-FRET ratio was taken as either the 520/490 nm (M344-FITC) or 640/548 nm (SAHA-NCT) intensity ratio. The assay floor (background) was defined with the 1 μM panobinostat dose, and the assay ceiling (top) was defined via a no-inhibitor control. Data were background corrected, normalized and fitted to a four-parameter dose response model using Prism 8.

Cell permeability profiling of Cora-2-Halo toward HDAC1-HaloTag construct as determined via competition of TMR-Halo labeling and SDS-PAGE analysis: HEK293T cells from donor dish were trypsinized, trypsin was neutralized with DMEM+10% FBS, cells were counted, and cell density was adjusted to 450.000 cells/mL. Separately, stock solutions of plasmid DNA (pFC14A-HDAC1-HaloTag; 16 μg/mL) and PET-MAX (48 μg/mL) were prepared in PBS such that the final volume of each stock solution was 1:20 of the volume of suspension cells in culture media (1:3 w/w DNA:PEI-MAX). The solutions were thoroughly mixed, then the DNA solution was added slowly to the PEI solution and the resulting transfection cocktail (1:10 volume of suspension) was incubated for 20 min at room temperature. The transfection cocktail was added to the cell suspension (final concentrations: 0.8 μg/mL DNA, 2.4 μg/mL PEI-MAX, 400,000 cells/mL) and 0.45 mL suspension was added to wells of a 24-well plate (Corning; 180,000 cells per well).

For 24 h treatments in cell culture medium, 50 μL of 1Ox Cora-2-Halo, Ac-Halo, or DMSO solution was added to select wells to achieve concentrations of 12.5, 25, or 50 M Cora-2-Halo, 50 μM Ac-Halo or 0.5% DMSO control. Cells were grown for 24 h at 37° C. and 5% CO₂.

After, media was aspirated from select untreated wells and replaced with 0.5 mL phenol red-free Opti-MEM (Gibco) containing DMSO control (0.5%), 50 μM Ac-Halo ligand, or a varying dose of Cora-2-Halo (12.5, 25, or 50 μM). Cells were treated for 4 h at 37° C. and 5% CO₂ then media was aspirated from all wells and cells were washed with 0.5 mL DPBS. Cells were then lysed in lysis buffer (see above) supplemented with 10 μM TMR-Halo ligand for 1 h at room temperature. To the lysate was added 20 μL 4×LDS loading buffer followed by 8 μL 10× reducing agent and samples were heated to 85° C. for 7 min. 20 μL were loaded onto an SDS-PAGE gel and analyzed via Cy3 fluorescence imaging on a fluorescence gel scanner (Typhoon). TMR-Halo competition from Ac-Halo and Cora-2-Halo treatments was assessed relative to DMSO control.

Intracellular target engagement assay with HDAC1-HaloTag construct and Cora-2-Halo complex: HEK293T cells from donor dish were trypsinized, trypsin was neutralized with DMEM+10% FBS, cells were counted, and cell density was adjusted to 400,000 cells/mL in 10.8 mL media containing 12.5 μM Cora-2-Halo. Separately, stock solutions of plasmid DNA (pFC14A-HDAC1-HaloTag; 16 μg/mL) and PEI-MAX (48 μg/mL) were prepared in PBS such that the final volume of each stock solution was 1:20 of the volume of suspension cells in culture media (1:3 w/w DNA:PEI-MAX). The solutions were thoroughly mixed, then the DNA solution was added slowly to the PEI solution and the resulting transfection cocktail (1:10 volume of suspension) was incubated for 20 min at room temperature. The transfection cocktail was added to the cell suspension (final concentrations: 0.8 μg/mL DNA, 2.4 μg/mL PEI-MAX, 350,000 cells/mL) and the 12 mL suspension was added to a 10 cm dish. Cells were grown for 24 h at 37° C. and 5% CO₂.

After, media was aspirated and cells were quickly washed with 12 mL phenol red-free DMEM+10% FBS, followed by one 30 min wash at 37° C. and 5% CO₂. Cells were washed with PBS, trypsinized, counted, and pelleted. Cell culture medium was aspirated from the cell pellet and cells (now labeled with Cora-2-Halo) were resuspended to a density of 400,000 cells/mL in phenol red-free Opti-MEM (Gibco) containing either 250 nM or 1 μM NCT-SAHA tracer (0.25% or 1% DMSO, respectively). The 1 μM NCT-SAHA (1% DMSO) conditions were chosen to reflect the standard conditions used in an intracellular NanoBRET target engagement assay for HDAC1-NanoLuc FL (also see Promega TM483). 50 μL of cell suspension were plated into white 384-well plates (Corning 3574; 20,000 cells/well; 6 replicates). Test compounds were added in serial dilution (1:2 titration, 15-point, cₘₐₓ=100 M SAHA/Cpd-60/CI-994, and 25 μM panobinostat; 1% DMSO [1.25% or 2% final]) using a D300 digital dispenser and allowed to equilibrate for 4 h at 37° C. and 5% CO₂. TR-FRET measurements were acquired on a Tecan SPARK plate reader: 340/50 nm excitation, 548 nm (Tb) and 640 nm (NCT) emission, 100 μs delay, 400 μs integration. The TR-FRET ratio was taken as the 640/548 nm intensity ratio. The assay floor (background) was defined with the 25 μM panobinostat dose, and the assay ceiling (top) was defined via a no-inhibitor control. Data were background corrected, normalized and fitted to a four-parameter dose response model using Prism 8.

log D measurements: for the measurement of log D (pH 7.4), which is the partition coefficient at a specific pH, 1 μL of a 10 mM DMSO stock of a given terbium complex was injected into the lower, aqueous layer of a 500 μL/500 μL partition between 50 mM sodium phosphate buffer, pH 7.4+150 mM NaCl and 1-octanol in a 1.5 mL Eppendorf tube. The partition was vigorously vortexed for 1 min then the emulsified solution was centrifuged at 21,000×g for 30 seconds to separate the layers, 1 μL of each layer was then added to 39 μL of DMSO (triplicate measurements) in a white, 384-well plate (Corning 3572) and the fluorescence intensity at 548/10 nm (340/50 nm excitation, 100 μs delay, 400 μs integration) was measured on a SPARK plate reader (Tecan, Grödig, Austria). The log D (pH 7.4) was calculated using Equation 9:

$$log D ( pH 7.4 ) = log ( \frac{fl . int . octanol}{fl . int . PBS} ) \qquad ( 9 )$$

where fl. int. octanol/PBS correspond to the 548 nm fluorescence intensities measured for each layer (octanol or 50 mM sodium phosphate, pH 7.4+150 mM NaCl).

Stability profiling of select terbium complexes: a D300 digital dispenser (Hewlett-Packard; Palo Alto, CA) was used to dispense terbium complexes (Cora-1/2/3-Halo, 12-14) into 50 μL of respective buffer solution in a white, 384-well plate (Corning 3572, triplicate measurements) to a final concentration of 5 nM. Initial fluorescence reads were taken on a Tecan SPARK plate reader (340/50 nm excitation. 548/10 nm emission, 100 μs delay, 400 μs integration). The plates were left at room temperature (sealed in between measurements), and fluorescence emission reads were taken over the course of 7 d to monitor the decrease in intensity with respect to time.

Bacterial protein expression: HaloTag-SNAP-tag-6×His fusion protein (HSFP6×His): plasmid pGW-Halo-SNAP-6×His (custom cloned by Genscript, Piscataway, New Jersey) was transformed into chemically competent BL21 Star™ (DE3) pLysS One Shot™ E. coli cells (ThermoFisher C602003). A single transformed colony from a Luria-Bertani (LB)-Ampicillin agar plate was used to inoculate 10 mL of LB Broth (MilliporeSigma™ 71-753-5) containing Ampicillin (0.1 mg/mL) and the culture was incubated at 37° C. overnight at 225 rpm. The following day, I mL of starter culture was used to inoculate 100 mL LB Broth containing Ampicillin (0.1 mg/mL), which was incubated at 37° C. with shaking at 225 rpm until OD₆₀₀ reached ˜0.3. The culture was cooled to 20° C. in an ice/water slurry then expression of HSFP6×His was induced with 0.3 mM Isopropyl β-d-1-thiogalactopyranoside (IPTG) and growth was continued for 16 h at 20° C. with agitation at 225 rpm. Cells were harvested by centrifugation at 3,000×g for 20 minutes at 4° C. and washed once with Dulbecco's PBS (DPBS). Cell pellets were snap-frozen in liquid nitrogen and stored at −80° C. until lysis was performed.

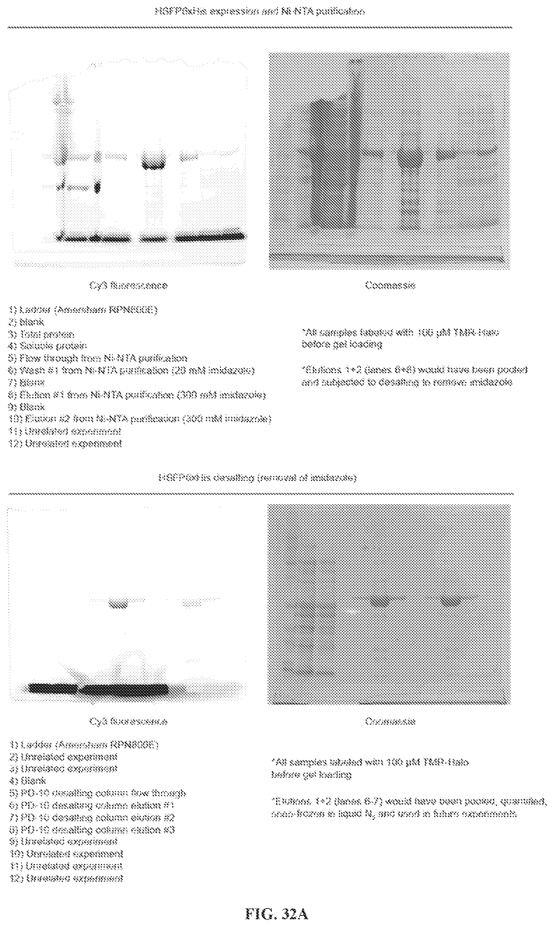

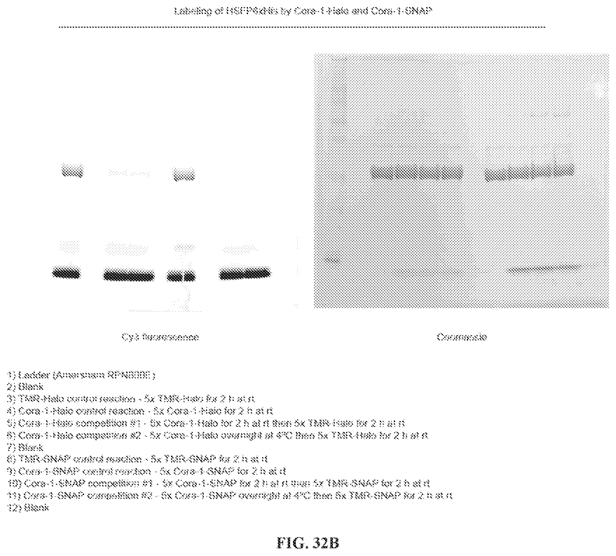

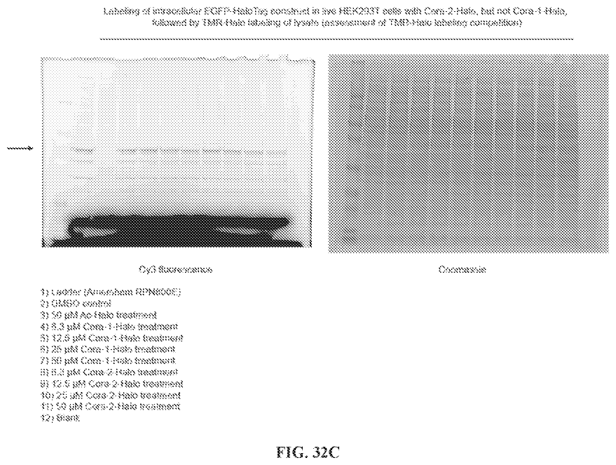

Frozen cell pellets were quickly thawed in a room temperature water bath and the cells were lysed with 2-3 pellet volumes of ice-cold lysis buffer [per 10 mL: 25 mM HEPES, 500 mM NaCl, 10 mM MgSO₄, 10% glycerol, 10 mM imidazole, 1 mM dithiothreitol (DTT), 1 mM 4-(2-aminoethyl)benzenesulfonyl fluoride hydrochloride (AEBSF, MilliporeSigma™ 10-150-050MG), 2 mM ATP, 250 U benzonase nuclease (Sigma E1014), and 90,000 U lysozyme (Lucigen R1804M) in B-PER™ (ThermoFisher 78248). pH 7.0]. Cells were homogenized with a serological pipette and kept on ice for 10 min, then the insoluble fraction was removed via centrifugation at 3,000×g for 20 min at 4° C. The soluble fraction was taken and incubated with 250 μL Ni-NTA resin (ThermoFisher 88223) that had been pre-equilibrated with wash buffer (25 mM HEPES, 500 mM NaCl, 10 mM Imidazole, 1 mM DTT, 10% glycerol, pH 7.0) for 1 h at 4° C. with slow, end-over-end mixing. The fraction containing non-bound proteins was removed via centrifugation at 700×g for 2 min. The resin was washed 2×400 μL with wash buffer containing 20 mM imidazole followed by 3×400 μL with wash buffer containing 40 mM imidazole. The bound protein was eluted with 2×400 μL elution buffer containing 300 mM imidazole. Small aliquots (˜5-10 μL) of wash/elution fractions were labeled with 100 μM TMR-Halo for 10 min at room temperature and subjected to SDS-PAGE analysis to check for purity (1.0 mm NuPAGE™ 4-12% Bis-Tris Protein Gels, NuPAGE™ MOPS running buffer, 140 V). Fluorescence gel images were taken on a Typhoon FLA 9500 scanner (Cy3 excitation/emission) and gels were subsequently stained with SimplyBlue™ SafeStain (ThermoFisher). Desired elution fractions were pooled and buffer exchanged into storage buffer (25 mM HEPES, 100 mM NaCl, 1 mM DTT, 5% glycerol, pH 7.0) using a PD-10 desalting column (GE 17-0851-01). Elutions were monitored via Nanodrop and desired fractions were pooled. The glycerol content was adjusted to 20%, then aliquots were snap-frozen in liquid nitrogen and stored at −80° C. until further use. See FIG. 32A.

HSFP6×His labeling: to 100 μL aliquots of HSFP6×His at a concentration of 0.72 mg/mL (13 μM, MW 56.3 kDa) in storage buffer (25 mM HEPES, 100 mM NaCl, 1 mM DTT, 20% glycerol, pH 7.0) were added the appropriate volume of FITC-Halo, TMR-Halo, Cora-1-SNAP, or combinations of FITC-Halo/Cora-1-SNAP and TMR-Halo/Cora-1-SNAP (10 mM DMSO stock solutions) to achieve a molar ratio of ˜5× dye(s) to protein (final DMSO content <5%). The reaction mixtures were briefly vortexed and allowed to react for 16 h at 4° C. To purify the labeled conjugates away from organic solvent and unreacted dye derivatives, the labeling reaction was buffer exchanged into fresh storage buffer using a 0.5 mL, 7K MWCO Zeba™ Spin Desalting Column according to the manufacturer's protocol. The corrected A₂₈₀ values (A_{280,corr}) of protein conjugates were determined via Nanodrop (0.1 cm path length) by measuring A₂₈₀, along with A₃₄₀ (Th), A₄₉₄ (FITC), and/or A₅₅₄ (TMR). Modifications of Equation 4 were used to solve for A_{280,corr} using correction factors of 0.157 (A₂₈₀/A₃₄₀) for Tb, 0.288 (A₂₈₀/A₄₉₄) for FITC, and 0.209 (A₂₈₀/A₅₅₄) for TMR. The extinction coefficient for HSFP6×His at A₂₈₀ is 82,640 M-‘cm’. Aliquots were snap-frozen in liquid nitrogen and stored at −80° C. until further use. See FIG. 32B.

HSFP6×His competition experiments via SDS-PAGE: to 20 μL aliquots of HSFP6×His at a concentration of 0.72 mg/mL (13 M, MW 56.3 kDa) in storage buffer (25 mM HEPES. 100 mM NaCl, 1 mM DTT, 20% glycerol, pH 7.0) were added the appropriate volume of TMR-Halo or Cora-1-Halo to achieve a molar ratio of ˜5× dye to protein (two labeling reactions were prepared for Cora-1-Halo). In parallel, labeling reactions were set up with TMR-SNAP or Cora-1-SNAP (again two labeling reactions prepared for Cora-1-SNAP). The labeling reactions were allowed to proceed for 2 h at room temperature. After, to one of the duplicate Cora-1-Halo or SNAP labeling reactions was added TMR-Halo or TMR-SNAP, respectively, at a molar ratio of 5× dye to protein and the reactions were incubated for an additional 2 h at room temperature. 10 μL of each reaction solution were subjected to SDS-PAGE analysis followed by Cy3 fluorescence imaging and Coomassie staining to assess the ability of the HaloTag (Cora-1-Halo) and SNAP-tag (Cora-1-SNAP) complexes to compete for TMR-Halo/TMR-SNAP binding to HSFP6×His. The Tb complexes are not detectable with Cy3 fluorescence imaging.

HSFP6×His TR-FRET experiments: HSFP6×His purified conjugates labeled with Cora-1-SNAP and either FITC-Halo or TMR-Halo were diluted to 2.5 nM into buffer containing 50 mM Tris, 1 mM DTT, 0.05% v/v TWEEN-20, pH 7.4 in a white, 384-well plate (Corning 3572, 30 μL assay volume, triplicate measurements). To one set of wells was added HisTEV (˜50× amount required to cleave amount of HSFP6×His in each well according to manufacturer's protocol, Genscript Z03030) and the plate was incubated at 30° C. for I h. Cleavage of the TEV protease site between the two proteins results in separation of donor and acceptor fluorophore and, as a result, diminishes TR-FRET capacity. TR-FRET measurements were acquired on a Tecan SPARK plate reader: 340/50 nm excitation, 490/10 nm (Tb) and either 520/10 nm (FITC) or 568/10 nm (TMR) emission, 100 μs delay, 400 μs integration. The TR-FRET ratio was taken as either the 520/490 nm intensity ratio (FITC) or 568/490 nm intensity ratio (TMR).

Tb-Anti-6×His (Abcam, 18184) validation with HSFP6×His: HSFP6×His purified conjugates labeled with either FITC-Halo or TMR-Halo were diluted to 2.5 nM into buffer containing 50 mM Tris, 1 mM DTT, 0.05% v/v TWEEN-20, pH 7.4 in a white, 384-well plate (Corning 3572, 30 μL assay volume, triplicate measurements). To one set of wells was added HisTEV (˜50× amount required to cleave amount of HSFP6×His in each well according to manufacturer's protocol, Genscript Z03030) and the plate was incubated at 30° C. for 1 h. Cleavage of the TEV protease site between the two proteins results in separation of the C-terminal 6×His tag (donor binding site) and acceptor fluorophore and, as a result, diminishes TR-FRET capacity. To each well was then added 0.1 nM Tb-Anti-6× His conjugate (Abeam, 18184) and the plate was incubated at room temperature for 1 h. TR-FRET measurements were acquired on a Tecan SPARK plate reader: 340/50 nm excitation, 490/10 nm (Tb) and either 520/10 nm (FITC) or 568/10 nm (TMR) emission, 100 μs delay, 400 μs integration. The TR-FRET ratio was taken as either the 520/490 nm intensity ratio (FITC) or 568/490 nm intensity ratio (TMR).

Equilibrium dissociation constant (K_{d}) measurements for Tb-Anti-6×His conjugates: Tb-Anti-6×His terbium conjugate (Abcam, 18184 or BioXCell, RT0266) was diluted to 0.5 nM into buffer containing 50 mM HEPES. 100 mM KCl, 0.5 mg/mL BSA, 0.001% (v/v) Tween-20, pH 7.5 in a white, 384-well plate (Corning 3572, 30 μL assay volume, triplicate measurements). A D300 digital dispenser was used to dispense a dose titration of HSFP6×His purified conjugate labeled with FITC-Halo from 0 nM to 125 nM (1:2 titration, 15-point). The plate was incubated for 2 h at room temperature then TR-FRET measurements were acquired on a Tecan SPARK plate reader: 340/50 nm excitation, 490/10 nm (T1) and 520/10 nm (FITC) emission, 100 μs delay, 400 μs integration. The TR-FRET ratio was taken as the 520/490 nm intensity ratio. Nonspecific signal was determined from wells that had been pre-treated with 50× HisTEV protease (Genscript Z03030) for 2 h at room temperature. Prism 8 was used to fit the data to a One Site—Specific Binding model.

Equilibrium dissociation constant (K_{d}) measurements for Tb-HaloTrap and AF488-HaloTrap conjugates: Tb-HaloTrap: Tb-HaloTrap conjugate (ChromoTek OT-250) was diluted to 0.5 nM into buffer containing 50 mM HEPES, 100 mM KCl, 0.5 mg/mL BSA, 0.001% (v/v) Tween-20, pH 7.5 in a white. 384-well plate (Corning 3572, 30 μL assay volume, duplicate measurements). A D300 digital dispenser was used to dispense a dose titration of HSFP6×His purified conjugate labeled with FITC-Halo from 0 nM to 125 nM (1:2 titration, 15-point). The plate was incubated for 2 h at room temperature then TR-FRET measurements were acquired on a Tecan SPARK plate reader: 340/50 nm excitation, 490/10 nm (Tb) and 520/10 nm (FITC) emission, 100 μs delay, 400 μs integration. The TR-FRET ratio was taken as the 520/490 nm intensity ratio. Prism 8 was used to fit the data to a One Site—Total Binding model.

AF488-HaloTrap: HSFP6×His purified conjugate labeled with Cora-1-SNAP was diluted to 0.5 nM into buffer containing 50 mM HEPES, 100 mM KCl, 0.5 mg/mL BSA, 0.001% (v/v) Tween-20, pH 7.5 in a white, 384-well plate (Corning 3572, 30 μL assay volume, duplicate measurements). A D300 digital dispenser was used to dispense a dose titration of AF488-HaloTrap (1:2 titration, 15-point). The plate was incubated for 2 h at room temperature then TR-FRET measurements were acquired on a Tecan SPARK plate reader: 340/50 nm excitation, 490/10 nm (Tb) and 520/10 nm (AF488) emission, 100 μs delay, 400 μs integration. The TR-FRET ratio was taken as the 520/490 nm intensity ratio. Prism 8 was used to fit the data to a One Site—Total Binding model.

Inhibitor dose responses with full length Keap1 (His/GST) construct and FITC-KL9/CDDO-FITC assay systems: Keap1 (His/GST; 11981-H20B; Sino Biological Inc.) was diluted to 1 nM into buffer containing 25 mM HEPES. 150 mM NaCl, 1 mM DTT, 0.5 mg/mL BSA, 0.005% (v/v) TWEEN-20, pH 7.4 with 0.5 nM Tb-Anti-6×His (Abcam, 18184) and either 5 nM FITC-KL9 (Assay 4) or 40 nM CDDO-FITC (Assay 3) in a white, 384-well plate (Corning 3572, 30 μL assay volume, triplicate measurements). A D300 digital dispenser was used to dispense dose titrations of competitor ligands, with maximal doses of 10 μM KI-696/Ac-KL9 and 100 μM N-KL9/OBT/CDDO/CDDO-Me/CDDO-JQ1/Celastrol/dimethyl fumarate in the FITC-KL9 assay or maximal doses of 10 M CDDO/CDDO-Me/CDDO-JQ1 and 100 μM KI-696/Ac-KL9/N-KL9/OBT/Celastrol/dimethyl fumarate in the CDDO-FITC assay (1:2 titrations, 15-point). Wells were incubated for 4 h at room temperature then TR-FRET measurements were acquired on a Tecan SPARK plate reader: 340/50 nm excitation, 490/10 nm (Tb) and 520/10 nm (FITC) emission, 100 μs delay, 400 μs integration. The TR-FRET ratio was taken as the 520/490 nm intensity ratio. The assay floor (background) was defined with the 10 μM KI-696 dose in the FITC-KL9 assay; the assay floor was defined with the 10 μM CDDO dose in the CDDO-FITC assay. The assay ceiling (top) was defined via a no-inhibitor control. Data were background corrected, normalized and Prism 8 was used to fit the data to a four-parameter dose response curve.

HDAC1 activity assay with recombinant protein: recombinant HDAC1 (His/FLAG; 50051; BPS Bioscience Inc, San Diego, CA) was diluted to 6 nM (1.2×) into buffer containing 50 mM HEPES, 100 mM KCl, 0.5 mg/mL BSA, 0.001% (v/v) Tween-20, pH 7.5 in a white, 384-well plate (Corning 3572, 25 μL initial assay volume, triplicate measurements). A D300 digital dispenser was used to dispense dose titrations of HDAC inhibitors/tracers, with maximal doses of 10 μM SAHA/Cpd-60/SAHA-NCT/M344-FITC, 1 μM panobinostat, and 100 μM CI-994 (1:2 titrations, 15-point). The plate was incubated for 3 h at room temperature, then 5 μL of 6× MAZ1600 HDAC substrate was added (final concentration 18 μM, 3×K_{M}) and deacetylase activity was allowed to proceed for 45 min at room temperature. After, 5 μL of 7× developer solution was added (150 nM trypsin+40 μM SAHA final concentrations) and the plate was incubated for 30 min at room temperature. 7-Amino-4-methyl coumarin fluorescence was measured on a Tecan SPARK plate reader: 350/20 nm excitation, 460/10 nm emission. The assay floor (background) was defined with the 1 μM panobinostat dose, and the assay ceiling (top) was defined via a no-inhibitor control. Data was background corrected, normalized and Prism 8 was used to fit the data to a four-parameter dose response curve.

Equilibrium dissociation constant (K_{d}) measurements for SAHA-NCT and M344-FITC toward HDAC1 recombinant protein: HDAC1 (His/FLAG; 50051; BPS Biosciences Inc) was diluted to 5 nM with 2.5 nM Tb-Anti-6×His (Abcam, 18184) in buffer containing 50 mM HEPES, 100 mM KCl, 0.5 mg/mL BSA, 0.001% (v/v) Tween-20, pH 7.5 in a white, 384-well plate (Corning 3572, 30 μL assay volume, triplicate measurements). A D300 digital dispenser was used to dispense dose titrations of either SAHA-NCT or M344-FITC (1:2 titration, 15-point) from 0 nM to 250 nM. Wells were incubated for 2 h at room temperature then TR-FRET measurements were acquired on a Tecan SPARK plate reader: 340/50 nm excitation, 490/10 nm (Tb) and 520/10 nm (FITC) for M344-FITC emission, 548 nm (Tb) and 640 nm (NCT) for SAHA-NCT emission, 100 μs delay, 400 μs integration. The TR-FRET ratio was taken as either the 520/490 nm (M344-FITC) or 640/548 nm (SAHA-NCT) intensity ratio. Nonspecific signal was determined from wells that had been treated with 50 μM SAHA. Prism 8 was used to fit the data to a One Site—Specific Binding model.

Inhibitor dose responses using recombinant HDAC1 protein and SAHA-NCT/M344-FITC assay systemsB: HDAC1 (His/FLAG; 50051; BPS Biosciences Inc) was diluted to 5 nM with 2.5 nM Tb-Anti-6×His (Abcam, 18184) and either 20 nM SAHA-NCT or 70 nM M344-FITC in buffer containing 50 mM HEPES, 100 mM KCl, 0.5 mg/mL BSA, 0.001% (v/v) Tween-20, pH 7.5 in a white, 384-well plate (Corning 3572, 30 μL assay volume, triplicate measurements). A D300 digital dispenser was used to dispense dose titrations of HDAC inhibitors, with maximal doses of 10 μM SAHA/Cpd-60, 1 μM panobinostat, and 100 μM CI-994 (1:2 titrations, 15-point). Wells were incubated for 3 h at room temperature then TR-FRET measurements were acquired on a Tecan SPARK plate reader: 340/50 nm excitation, 490/10 nm (Tb) and 520/10 nm (FITC) for M344-FITC emission, 548 nm (Tb) and 640 nm (NCT) for SAHA-NCT emission, 100 μs delay, 400 μs integration. The TR-FRET ratio was taken as either the 520/490 nm (M344-FITC) or 640/548 nm (SAHA-NCT) intensity ratio. The assay floor (background) was defined with the 1 μM panobinostat dose, and the assay ceiling (top) was defined via a no-inhibitor control. Data was background corrected, normalized and Prism 8 was used to fit the data to a four-parameter dose response curve.

Equilibrium dissociation constant (K_{d}) measurements for SAHA-NCT and M344-FITC toward HDAC1-HT (Cora-1-Halo labeled) in cell lysate: Cora-1-Halo labeled HDAC1-HT cell lysate was diluted to 1 nM in HDAC1-HT in buffer containing 50 mM HEPES, 100 mM KCl, 0.5 mg/mL BSA, 0.001% (v/v) Tween-20, pH 7.5 in a white, 384-well plate (Corning 3572, 30 μL assay volume, triplicate measurements). The total protein concentration was 15 μg/mL. A D300 digital dispenser was used to dispense dose titrations of either SAHA-NCT or M344-FITC (1:2 titration, 15-point) from 0 nM to 250 nM. Wells were incubated for 2 h at room temperature then TR-FRET measurements were acquired on a Tecan SPARK plate reader: 340/50 nm excitation, 490/10 nm (Tb) and 520/10 nm (FITC) for M344-FITC emission, 548 nm (Tb) and 640 nm (NCT) for SAHA-NCT emission, 100 μs delay, 400 μs integration. The TR-FRET ratio was taken as either the 520/490 nm (M344-FITC) or 640/548 nm (SAHA-NCT) intensity ratio. Nonspecific signal was determined from wells that had been treated with 50 μM SAHA. Prism 8 was used to fit the data to a One Site—Specific Binding model.

Cell permeability profiling of CoraFluors toward cytosolic HA-EGFP-HaloTag2 construct as determined via competition of TMR-Halo labeling and SDS-PAGE analysis: HEK293T cells from donor dish were trypsinized, trypsin was neutralized with DMEM+10% FBS, cells were counted, and cell density was adjusted to 450.000 cells/mL. Separately, stock solutions of plasmid DNA (HA-EGFP-HaloTag2—Addgene 41742; 16 μg/mL) and PEI-MAX (48 μg/mL) were prepared in PBS such that the final volume of each stock solution was 1:20 of the volume of suspension cells in culture media (1:3 w/w DNA:PEI-MAX). The solutions were thoroughly mixed, then the DNA solution was added slowly to the PEI solution and the resulting transfection cocktail (1:10 volume of suspension) was incubated for 20 min at room temperature. The transfection cocktail was added to the cell suspension (final concentrations: 0.8 μg/mL DNA, 2.4 μg/mL PEI-MAX, 400,000 cells/mL) and 0.45 mL suspension was added to wells of a 24-well plate (Corning; 180,000 cells per well). Cells were grown for 24 h at 37° C. and 5% CO₂.

After, media was aspirated and replaced with 0.4 mL phenol red-free Opti-MEM (Gibco) containing DMSO control (0.5%), 50 μM Ac-Halo ligand, or a varying dose of Cora-1-Halo or Cora-2-Halo (6.3, 12.5, 25, or 50 μM). Cells were treated for 4 h at 37° C. and 5% CO₂ then media was aspirated and cells were washed with 0.5 mL DPBS. Cells were then lysed in lysis buffer (50 μL) containing 50 mM Tris, 150 mM NaCl, 2 mM DTT, 1% (v/v) Triton X-100, 0.1% (w/v) sodium deoxycholate, pH 7.5 supplemented with 250 U benzonase, lx protease inhibitor cocktail (Promega G6521), and 10 μM TMR-Halo ligand for 1 h at room temperature. To the lysate was added 20 μL 4×LDS loading buffer followed by 8 μL 10× reducing agent and samples were heated to 85° C. for 7 min. 20 μL were loaded onto an SDS-PAGE gel and analyzed via Cy3 fluorescence imaging on a Typhoon scanner. TMR-Halo competition from Ac-Halo and Cora-I/Cora-2-Halo treatments was assessed relative to DMSO control. See FIG. 32C.

Detection of intracellular TR-FRET signal between Cora-2-Halo and HA-EGFP-HaloTag2: HEK293T cells from donor dish were trypsinized, trypsin was neutralized with DMEM+10% FBS, cells were counted, and cell density was adjusted to 325,000 cells/mL. Separately, stock solutions of plasmid DNA (HA-EGFP-HaloTag2; 16 μg/mL) and PEI-MAX (48 μg/mL) were prepared in PBS such that the final volume of each stock solution was 1:20 of the volume of suspension cells in culture media (1:3 w/w DNA:PEI-MAX). The solutions were thoroughly mixed, then the DNA solution was added slowly to the PEI solution and the resulting transfection cocktail (1:10 volume of suspension) was incubated for 20 min at room temperature. The transfection cocktail was added to the cell suspension (final concentrations: 0.8 μg/mL DNA, 2.4 μg/mL PEI-MAX, 300,000 cells/mL) then 2.5 mL suspension was added to wells of a 6-well plate (Corning; 750,000 cells per well). For non-transfected cell control, an identical volume of PBS alone was added to a separate suspension and cells were plated identically. Cells were grown for 24 h at 37° C. and 5% CO₂.

After, media was aspirated and replaced with 1 mL phenol red-free Opti-MEM (Gibco) containing 50 μM Ac-Halo ligand+50 μM Cora-2-Halo (1 h pre-treatment with Ac-Halo before Cora-2-Halo addition), or 50 μM Cora-2-Halo alone (for both transfected & non-transfected cells). Cells were treated for 4 h at 37° C. and 5% CO₂ then media was aspirated and cells were quickly washed with 3 mL phenol red-free Opti-MEM, followed by two 30 min washes at 37° C. Media was aspirated and cells were trypsinized, then trypsin was neutralized with phenol red-free DMEM+10% FBS and cells were counted. Cells were pelleted at 200×g for 5 min, media was aspirated and cell density was adjusted to 1,000,000 cells/mL in phenol red-free Opti-MEM. 50 μL of cell suspension were plated into a 384w plate (Corning 3574; 50,000 cells/well; 16 replicates) and TR-FRET measurements were acquired on a Tecan SPARK plate reader: 340/50 nm excitation, 490/10 nm (Th) and 520/10 nm (EGFP) emission, 100 μs delay. 400 μs integration. Because terbium signal was dynamic due to excess, free Cora-2-Halo being washed out of pre-blocked (Ac-Halo) and non-transfected cells, the 520 nm FRET-sensitized EGFP emission signal was plotted and positive signal was compared to control conditions (Ac-Halo pre-block and non-transfected control).

Optimization of tracer dose for intracellular target engagement assay with HDAC1-HaloTag construct and Cora-2-Halo complex: HEK293T cells from donor dish were trypsinized, trypsin was neutralized with DMEM+10% FBS, cells were counted, and cell density was adjusted to 400,000 cells/mL in 10.8 mL media containing 12.5 μM Cora-2-Halo. Separately, stock solutions of plasmid DNA (pFC14A-HDAC1-HaloTag; 16 μg/mL) and PET-MAX (48 μg/mL) were prepared in PBS such that the final volume of each stock solution was 1:20 of the volume of suspension cells in culture media (1:3 w/w DNA:PEI-MAX). The solutions were thoroughly mixed, then the DNA solution was added slowly to the PEI solution and the resulting transfection cocktail (1:10 volume of suspension) was incubated for 20 min at room temperature. The transfection cocktail was added to the cell suspension (final concentrations: 0.8 μg/mL DNA, 2.4 μg/mL PET-MAX, 350,000 cells/mL) and the 12 mL suspension was added to a 10 cm dish. Cells were grown for 24 h at 37° C. and 5% CO₂.

After, media was aspirated and cells were quickly washed with 12 mL phenol red-free DMEM+10% FBS, followed by one 30 min wash at 37° C. and 5% CO₂. Cells were washed with PBS, trypsinized, counted, and pelleted. Cell culture medium was aspirated from the cell pellet and cells (now labeled with Cora-2-Halo) were resuspended to a density of 400,000 cells/mL in phenol red-free Opti-MEM (Gibco). The cell suspension was split in two and to one batch was added DMSO control (0.25%) and to the other 25 μM panobinostat. 50 μL of either cell suspension were plated into a 384w plate (Corning 3574; 20,000 cells/well: 6 replicates). A D300 digital dispenser was used to dispense dose titrations of SAHA-NCT and M344-FITC from 0 μM to 2 μM (1:1.5 titrations, 15-point, 2% DMSO maximum). The plate was incubated for 2 h (and 4 h) at 37° C. and 5% CO₂ then TR-FRET measurements were acquired on a Tecan SPARK plate reader: 340/50 nm excitation. 490/10 nm (Tb) and 520/10 nm (FITC) for M344-FITC emission, 548 nm (Tb) and 640 nm (NCT) for SAHA-NCT emission, 100 μs delay, 400 μs integration. The TR-FRET ratio was taken as either the 520/490 nm (M344-FITC) or 640/548 nm (SAHA-NCT) intensity ratio. Signal to background on a per-dose basis was calculated relative to 25 μM panobinostat control.

After live-cell measurements were taken, 25 μL of lysis buffer containing 50 mM Tris, 150 mM NaCl, 2 mM DTT, 1% v/v Triton X-100, 0.1% w/v sodium deoxycholate, pH 7.5 was added to all wells of the 384w plate. The plate was spun at 3000×g for 3 min and was further incubated for 2 h before identical TR-FRET measurements were taken. Because cell membranes were compromised. the biochemically validated, but cell impermeable FITC-M344 tracer now showed positive TR-FRET signal that could be abolished by 25 μM panobinostat.

Whole plasmid sequencing: whole-plasmid sequencing was performed by Massachusetts General Hospital Center for Computational and Integrative Biology core facility, Boston, MA. See below for plasmid sequences.

pGW-Halo-SNAP-6xHis (HSFP6xHis)
TCACTGATTAAGCATTGGTAATTCGAAATGACCGACCAAGCGACGCCCAACCGGTATCAGCTCACTCAAAGGCGGTAATA
CGGTTATCCACAGAATCAGGGGATAACGCAGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAATCGACGCTCAAGTCAGAGGTGGC
GAAACCCGACAGGACTATAAAGATACCAGGCGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTCTCATAGCTCACGCTGTAGGTATCTCAGTTC
GGTGTAGGTCGTTCGCTCCAAGCTGGGCTGTGTCACGAACCCCCCGTTCAGCCCGACCGCTGCGCCTTATCCGGTAACTA
TCGTCTTGAGTCCAACCCGGTAAGACACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAGAAGGACAGTATTTGGTATCTGCGCT
CTGCTGAAGCCAGTTACCTTCGGAAAAAGAGTTGGTAGCTCTTGATCCGGCAAACAAACCACCGCTGGTAGCGGTGGTTT
TTTTGTTTGCAAGCAGCAGATTACGCGCAGAAAAAAAGGATTTCAAGAAGATCCTTTGATCTTTTCTACGGGGTCTGACG
CTCAGTGGAACGAAAACTCACGTTAAGGGATTTTGGTCATGAGATTATCAAAAAGGATCTTCACCTAGATCCTTTTATAG
TCCGGAAATACAGGAACGCACGCTGGATGGCCCTTCGCTGGGATGGTGAAACCATGAAAAATGGCAGCTTCAGTGGATTA
AGTGGGGGTAATGTGGCCTGTACCCTCTGGTTGCATAGGTATTCATACGGTTAAAATTTATCAGGCGCGATTGCGGCAGT
TTTTCGGGTGGTTTGTTGCCATTTTTACCTGTCTGCTGCCGTGATCGCGCTGAACGCGTTTTAGCGGTGCGTACAATTAA
GGGATTATGGTAAATCCACTTACTGTCTGCCCTCGTAGCCATCGAGATAAACCGCAGTACTCCGGCCACGATGCGTCCGG
CGTAGAGGATCGAGATCTTTTCAGCCTGATACAGATTAAATCAGAACGCAGAAGCGGTCTGATAAAACAGAATTTGCCTG
GCGGCAGTAGCGCGGTGGTCCCACCTGACCCCATGCCGAACTCAGAAGTGAAACGCCGTAGCGCCGATGGTAGTGTGGGG
TCTCCCCATGCGAGATAGGGAACTGCCAGGCATCAAATAAAACGAAAGGCTCAGTCGAAAGACTGGGCCTTTCGTTTTAT
CTGTTGTTTGTCGGTGAACGCTCTCCTGAGTAGGACAAATCCGCCGGGAGCGGATTTGAACGTTGCGAAGCAACGGCCCG
GAGGGTGGCGGGCAGGACGCCCGCCATAAACTGCCAGGCATCAAATTAAGCAGAAGGCCATCCTGACGGATGGCCTTTTT
GCGTTTCTACAAACTCTTTTGTTTATTTTTCTAAATACATTCAAATATGTATCCGCTCATGGATCTCGATCCCGCGAAAT
TAATACGACTCACTATAGGGTATTTTAACTTTACTAAGGAGAATTCATCATGGCAGAAATCGGTACTGGCTTTCCATTCG
ACCCCCATTATGTGGAAGTCCTGGGCGAGCGCATGCACTACGTCGATGTTGGTCCGCGCGATGGCACCCCTGTGCTGTTC
CTGCACGGTAACCCGACCTCCTCCTACGTGTGGCGCAACATCATCCCGCATGTTGCACCGACCCATCGCTGCATTGCTCC
AGACCTGATCGGTATGGGCAAATCCGACAAACCAGACCTGGGTTATTTCTTCGACGACCACGTCCGCTTCATGGATGCCT
TCATCGAAGCCCTGGGTCTGGAAGAGGTCGTCCTGGTCATTCACGACTGGGGCTCCGCTCTGGGTTTCCACTGGGCCAAG
CGCAATCCAGAGCGCGTCAAAGGTATTGCATTTATGGAGTTCATCCGCCCTATCCCGACCTGGGACGAATGGCCAGAATT
TGCCCGCGAGACCTTCCAGGCCTTCCGCACCACCGACGTCGGCCGCAAGCTGATCATCGATCAGAACGTTTTTATCGAGG
GTACGCTGCCGATGGGTGTCGTCCGCCCGCTGACTGAAGTCGAGATGGACCATTACCGCGAGCCGTTCCTGAATCCTGTT
GACCGCGAGCCACTGTGGCGCTTCCCAAACGAGCTGCCAATCGCCGGTGAGCCAGCGAACATCGTCGCGCTGGTCGAAGA
ATACATGGACTGGCTGCACCAGTCCCCTGTCCCGAAGCTGCTGTTCTGGGGCACCCCAGGCGTTCTGATCCCACCGGCCG
AAGCCGCTCGCCTGGCCAAAAGCCTGCCTAACTGCAAGGCGTGGACATCGGCCCGGGTCTGAATCTGCTGCAAGAAGACA
ACCCGGACCTGATCGGCAGCGAGATCGCGCGCTGGCTGTCGACGCTCGAGATTTCCGGCGAGCCAACCACTGAGGATCTG
TACTTTCAGAGCATCACAAGTTTGTACAAAAAAGCAGGCTCCACCATGGACAAAGACTGCGAAATGAAGCGCACCACCCT
GGATAGCCCTCTGGGCAAGCTGGAACTGTCTGGGTGCGAACAGGGCCTGCACCGTATCATCTTCCTGGGCAAAGGAACAT
CTGCCGCCGACGCCGTGGAAGTGCCTGCCCCAGCCGCCGTGCTGGGCGGACCAGAGCCACTGATGCAGGCCACCGCCTGG
CTCAACGCCTACTTTCACCAGCCTGAGGCCATCGAGGAGTTCCCTGTGCCAGCCCTGCACCACCCAGTGTTCCAGCAGGA
GAGCTTTACCCGCCAGGTGCTGTGGAAACTGCTGAAAGTGGTGAAGTTCGGAGAGGTCATCAGCTACAGCCACCTGGCCG
CCCTGGCCGGCAATCCCGCCGCCACCGCCGCCGTGAAAACCGCCCTGAGCGGAAATCCCGTGCCCATTCTGATCCCCTGC
CACCGGGTGGTGCAGGGCGACCTGGACGTGGGGGGCTACGAGGGCGGGCTCGCCGTGAAAGAGTGGCTGCTGGCCCACGA
GGGCCACAGACTGGGCAAGCCTGGGCTGGGTCACCATCACCATCACCATTAGACCCAGCTTTCTTGTACAAAGTGGTGAT
TAACTAGTTGAGCGGCCGCGCATGCAAGCTGATCCGGCTGCTAACAAAGCCCGAAAGGAAGCTGAGTTGGCTGCTGCCAC
CGCTGAGCAATAACTAGCATAACCCCTTGGGGCCTCTAAACGGGTCTTGAGGGGTTTTTTGCTGAAAGGAGGAACTATAT
CCGGTTCGCTTGCTGTCCATAAAACCGCCCAGTCTAGCTATCGCCATGTAAGCCCACTGCAAGCTACCTGCTTTCTCTTT
GCGCTTGCGTTTTCCCTTGTCCAGATAGCCCAGTAGCTGACATTCATCCGGGGTCAGCACCGTTTCTGCGGACTGGCTTT
CTACGTAATGGTTTCTTAGACGTCAGGTGGCACTTTTCGGGGAAATGTGCGCGGAACCCCTATTTGTTTATTTTTCTAAA
TACATTCAAATATGTATCCGCTCATGAGACAATAACCCTGATAAATGCTTCAATAATATTGAAAAAGGAAGAGTATGAGT
ATTCAACATTTCCGTGTCGCCCTTATTCCCTTTTTTGCGGCATTTTGCCTTCCTGTTTTTGCTCACCCAGAAAGCGCTGG
TGAAAGTAAAAGATGCTGAAGATCAGTTGGGTGCACGAGTGGGTTACATCGAACTGGATCTCAACAGCGGTAAGATCCTT
GAGAGTTTTCGCCCCGAAGAACGTTTTCCAATGATGAGCACTTTCAAAGTTCTGCTATGTGGCGCGGTATTATCCCGTAT
TGACGCCGGGCAAGAGCAACTCGGTCGCCGCATACACTATTCTCAGAATGACTTGGTTGAGTACTCACCAGTCACAGAAA
AGCATCTTACGGATGGCATGACAGTAAGAGAATTATGCAGTGCTGCCATAACCATGAGTGATAACACTGCGGCCAACTTA
CTTCTGACAACTATCGGAGGACCGAAGGAGCTAACCGCTTTTTTGCACAACATGGGGGATCATGTAACTCGCCTTGATCG
TTGGGAACCGGAGCTGAATGAAGCCATACCAAACGACGAGCGTGACACCACGATGCCTGTAGCAATGGCAACAACGTTGC
GCAAACTATTAACTGGCGAACTACTTACTCTAGCTTCCCGGCAACAATTAATAGACTGGATGGAGGCGGATAAAGTTGCA
GGACCACTTCTGCGCTCGGCCCTTCCGGCTGGCTGGTTTATTGCTGATAAATCTGGAGCCGGTGAGCGTGGGTCTCGCGG
TATCATTGCAGCACTGGGGCCAGATGGTAAGCCCTCCCGTATCGTAGTTATCTACACGACGGGGAGTCAGGCAACTATGG
ATGAACGAAATAGACAGATCGCTGAGATAGGTGCCTCACTGATTAAGCATTGGTAATTCGAAATGACCGACCAAGCGACG
CCCAACC-3′
pFC14A-HDAC1-Halo (HDAC1-HT)
5′-TCAATATTGGCCATTAGCCATATTATTCATTGGTTATATAGCATAAATCAATATTGGCTATTGGCCATTGCATACGT
TGTATCTATATCATAATATGTACATTTATATTGGCTCATGTCCAATATGACCGCCATGTTGGCATTGATTATTGACTAGT
TATTAATAGTAATCAATTACGGGGTCATTAGTTCATAGCCCATATATGGAGTTCCGCGTTACATAACTTACGGTAAATGG
CCCGCCTGGCTGACCGCCCAACGACCCCCGCCCATTGACGTCAATAATGACGTATGTTCCCATAGTAACGCCAATAGGGA
CTTTCCATTGACGTCAATGGGTGGAGTATTTACGGTAAACTGCCCACTTGGCAGTACATCAAGTGTATCATATGCCAAGT
CCGCCCCCTATTGACGTCAATGACGGTAAATGGCCCGCCTGGCATTATGCCCAGTACATGACCTTACGGGACTTTCCTAC
TTGGCAGTACATCTACGTATTAGTCATCGCTATTACCATGGTGATGCGGTTTTGGCAGTACACCAATGGGCGTGGATAGC
GGTTTGACTCACGGGGATTTCCAAGTCTCCACCCCATTGACGTCAATGGGAGTTTGTTTTGGCACCAAAATCAACGGGAC
TTTCCAAAATGTCGTAATAACCCCGCCCCGTTGACGCAAATGGGCGGTAGGCGTGTACGGTGGGAGGTCTATATAAGCAG
AGCTGGTTTAGTGAACCGTCAGATCACTAGAAGCTTTATTGCGGTAGTTTATCACAGTTAAATTGCTAACGCAGTCAGTG
CTTCTGACACAACAGTCTCGAACTTAAGCTGCAGAAGTTGGTCGTGAGGCACTGGGCAGGTAAGTATCAAGGTTACAAGA
CAGGTTTAAGGAGACCAATAGAAACTGGGCTTGTCGAGACAGAGAAGACTCTTGCGTTTCTGATAGGCACCTATTGGTCT
TACTGACATCCACTTTGCCTTTCTCTCCACAGGTGTCCACTCCCAGTTCAATTACAGCTCTTAAGGCTAGAGTATTAATA
CGACTCACTATAGGGCTAGCGATCGCCATGGCGCAGACGCAGGGCACCCGGAGGAAAGTCTGTTACTACTACGACGGGGA
TGTTGGAAATTACTATTATGGACAAGGCCACCCAATGAAGCCTCACCGAATCCGCATGACTCATAATTTGCTGCTCAACT
ATGGTCTCTACCGAAAAATGGAAATCTATCGCCCTCACAAAGCCAATGCTGAGGAGATGACCAAGTACCACAGCGATGAC
TACATTAAATTCTTGCGCTCCATCCGTCCAGATAACATGTCGGAGTACAGCAAGCAGATGCAGAGATTCAACGTTGGTGA
GGACTGTCCAGTATTCGATGGCCTGTTTGAGTTCTGTCAGTTGTCTACTGGTGGTTCTGTGGCAAGTGCTGTGAAACTTA
TAAGCAGCAGACGGACATCGCTGTGAATTGGGCTGGGGGCCTGCACCATGCAAAGAAGTCCGAGGCATCTGGCTTCTGTT
ACGTCAATGATATCGTCTTGGCCATCCTGGAACTGCTAAAGTATCACCAGAGGGTGCTGTACATTGACATTGATATTCAC
CATGGTGACGGCGTGGAAGAGGCCTTCTACACCACGGACCGGGTCATGACTGTGTCCTTTCATAAGTATGGAGAGTACTT
CCCAGGAACTGGGGACCTACGGGATATCGGGGCTGGCAAAGGCAAGTATTATGCTGTTAACTACCCGCTCCGAGACGGGA
TTGATGACGAGTCCTATGAGGCCATTTTCAAGCCGGTCATGTCCAAAGTAATGGAGATGTTCCAGCCTAGTGCGGTGGTC
TTACAGTGTGGCTCAGACTCCCTATCTGGGGATCGGTTAGGTTGCTTCAATCTAACTATCAAAGGACACGCCAAGTGTGT
GGAATTTGTCAAGAGCTTTAACCTGCCTATGCTGATGCTGGGAGGCGGTGGTTACACCATTCGTAACGTTGCCCGGTGCT
GGACATATGAGACAGCTGTGGCCCTGGATACGGAGATCCCTAATGAGCTTCCATACAATGACTACTTTGAATACTTTGGA
CCAGATTTCAAGCTCCACATCAGTCCTTCCAATATGACTAACCAGAACACGAATGAGTACCTGGAGAAGATCAAACAGCG
ACTGTTTGAGAACCTTAGAATGCTGCCGCACGCACCTGGGGTCCAAATGCAGGCGATTCCTGAGGACGCCATCCCTGAGG
AGAGTGGCGATGAGGACGAAGACGACCCTGACAAGCGCATCTCGATCTGCTCCTCTGACAAACGAATTGCCTGTGAGGAA
GAGTTCTCCGATTCTGAAGAGGAGGGAGAGGGGGGCCGCAAGAACTCTTCCAACTTCAAAAAAGCCAAGAGAGTCAAAAC
AGAGGATGAAAAAGAGAAAGACCCAGAGGAGAAGAAAGAAGTCACCGAAGAGGAGAAAACCAAGGAGGAGAAGCCAGAAG
CCAAAGGGGTCAAGGAGGAGGTCAAGTTGGCCGTTTCTCTCGAGCCAACCACTGAGGATCTGTACTTTCAGAGCGATAAC
GATGGATCCGAAATCGGTACTGGCTTTCCATTCGACCCCCATTATGTGGAAGTCCTGGGCGAGCGCATGCACTACGTCGA
TGTTGGTCCGCGCGATGGCACCCCTGTGCTGTTCCTGCACGGTAACCCGACCTCCTCCTACGTGTGGCGCAACATCATCC
CGCATGTTGCACCGACCCATCGCTGCATTGCTCCAGACCTGATCGGTATGGGCAAATCCGACAAACCAGACCTGGGTTAT
TTCTTCGACGACCACGTCCGCTTCATGGATGCCTTCATCGAAGCCCTGGGTCTGGAAGAGGTCGTCCTGGTCATTCACGA
CTGGGGCTCCGCTCTGGGTTTCCACTGGGCCAAGCGCAATCCAGAGCGCGTCAAAGGTATTGCATTTATGGAGTTCATCC
GCCCTATCCCGACCTGGGACGAATGGCCAGAATTTGCCCGCGAGACCTTCCAGGCCTTCCGCACCACCGACGTCGGCCGC
AAGCTGATCATCGATCAGAACGTTTTTATCGAGGGTACGCTGCCGATGGGTGTCGTCCGCCCGCTGACTGAAGTCGAGAT
GGACCATTACCGCGAGCCGTTCCTGAATCCTGTTGACCGCGAGCCACTGTGGCGCTTCCCAAACGAGCTGCCAATCGCCG
GTGAGCCAGCGAACATCGTCGCGCTGGTCGAAGAATACATGGACTGGCTGCACCAGTCCCCTGTCCCGAAGCTGCTGTTC
TGGGGCACCCCAGGCGTTCTGATCCCACCGGCCGAAGCCGCTCGCCTGGCCAAAAGCCTGCCTAACTGCAAGGCTGTGGA
CATCGGCCCGGGTCTGAATCTGCTGCAAGAAGACAACCCGGACCTGATCGGCAGCGAGATCGCGCGCTGGCTGTCTACTC
TGGAGATTTCCGGTTAATAGAATTCTAGAGTCGACCTGCAGGCATGCAAGCTGATCCGGCTGCTAACAAAGCCCGAAAGG
AAGCTGAGTTGGCTGCTGCCACCGCTGAGCAATAACTAGCATAACCCCTTGGGGCGGCCGCTTCGAGCAGACATGATAAG
ATACATTGATGAGTTTGGACAAACCACAACTAGAATGCAGTGAAAAAAATGCTTTATTTGTGAAATTTGTGATGCTATTG
CTTTATTTGTAACCATTATAAGCTGCAATAAACAAGTTAACAACAACAATTGCATTCATTTTATGTTTCAGGTTCAGGGG
GAGATGTGGGAGGTTTTTTTAAGCAAGTAAAACCTCTACAAATGTGGTAAAATCGAATTCTAATGGATCCTCTTTGCGCT
TGCGTTTTCCCTTGTCCAGATAGCCCAGTAGCTGACATTCATCCGGGGTCAGCACCGTTTCTGCGGACTGGCTTTCTACG
TAATGGTTTCTTAGACGTCAGGTGGCACTTTTCGGGGAAATGTGCGCGGAACCCCTATTTGTTTATTTTTCTAAATACAT
TCAAATATGTATCCGCTCATGAGACAATAACCCTGATAAATGCTTCAATAATATTGAAAAAGGAAGAGTATGAGTATTCA
ACATTTCCGTGTCGCCCTTATTCCCTTTTTTGCGGCATTTTGCCTTCCTGTTTTTGCTCACCCAGAAACGCTGGTGAAAG
TAAAAGATGCTGAAGATCAGTTGGGTGCACGAGTGGGTTACATCGAACTGGATCTCAACAGCGGTAAGATCCTTGAGAGT
TTTCGCCCCGAAGAACGTTTTCCAATGATGAGCACTTTCAAAGTTCTGCTATGTGGCGCGGTATTATCCCGTATTGACGC
CGGGCAAGAGCAACTCGGTCGCCGCATACACTATTCTCAGAATGACTTGGTTGAGTACTCACCAGTCACAGAAAAGCATC
TTACGGATGGCATGACAGTAAGAGAATTATGCAGTGCTGCCATAACCATGAGTGATAACACTGCGGCCAACTTACTTCTG
ACAACTATCGGAGGACCGAAGGAGCTAACCGCTTTTTTGCACAACATGGGGGATCATGTAACTCGCCTTGATCGTTGGGA
ACCGGAGCTGAATGAAGCCATACCAAACGACGAGCGTGACACCACGATGCCTGTAGCAATGGCAACAACGTTGCGCAAAC
TATTAACTGGCGAACTACTTACTCTAGCTTCCCGGCAACAATTAATAGACTGGATGGAGGCGGATAAAGTTGCAGGACCA
CTTCTGCGCTCGGCCCTTCCGGCTGGCTGGTTTATTGCTGATAAATCTGGAGCCGGTGAGCGTGGGTCTCGCGGTATCAT
TGCAGCACTGGGGCCAGATGGTAAGCCCTCCCGTATCGTAGTTATCTACACGACGGGGAGTCAGGCAACTATGGATGAAC
GAAATAGACAGATCGCTGAGATAGGTGCCTCACTGATTAAGCATTGGTAATTCGAAATGACCGACCAAGCGACGCCCAAC
CGGTATCAGCTCACTCAAAGGCGGTAATACGGTTATCCACAGAATCAGGGGATAACGCAGGAAAGAACATGTGAGCAAAA
GGCCAGCAAAAGGCCAGGAACCGTAAAAAGGCCGCGTTGCTGGCGTTTTTCCATAGGCTCCGCCCCCCTGACGAGCATCA
CAAAAATCGACGCTCAAGTCAGAGGTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCCTTCGGGAAGCGTGGCGCTTTCT
CATAGCTCACGCTGTAGGTATCTCAGTTCGGTGTAGGTCGTTCGCTCCAAGCTGGGCTGTGTGCACGAACCCCCCGTTCA
GCCCGACCGCTGCGCCTTATCCGGTAACTATCGTCTTGAGTCCAACCCGGTAAGACACGACTTATCGCCACTGGCAGCAG
CCACTGGTAACAGGATTAGCAGAGCGAGGTATGTAGGCGGTGCTACAGAGTTCTTGAAGTGGTGGCCTAACTACGGCTAC
ACTAGAAGGACAGTATTTGGTATCTGCGCTCTGCTGAAGCCAGTTACCTTCGGAAAAAGAGTTGGTAGCTCTTGATCCGG
CAAACAAACCACCGCTGGTAGCGGTGGTTTTTTTGTTTGCAAGCAGCAGATTACGCGCAGAAAAAAAGGATTTCAAGAAG
ATCCTTTGATCTTTTCTACGGGGTCTGACGCTCAGTGGAACGAAAACTCACGTTAAGGGATTTTGGTCATGAGATTATCA
AAAAGGATCTTCACCTAGATCCTTTTATAGTCCGGAAATACAGGAACGCACGCTGGATGGCCCTTCGCTGGGATGGTGAA
ACCATGAAAAATGGCAGCTTCAGTGGATTAAGTGGGGGTAATGTGGCCTGTACCCTCTGGTTGCATAGGTATTCATACGG
TTAAAATTTATCAGGCGCGATGCGGCAGTTTTTCGGGTGGTTTGTTGCCATTTTTACCTGTCTGCTGCCGTGATCGCGCT
GAACGCGTTTTAGCGGTGCGTACAATTAAGGGATTATGGTAAATCCACTTACTGTCTGCCCTCGTAGCCATCGAGATAAA
CCGCAGTACTCCGGCCACGATGCGTCCGGCGTAGAGGATCGAGATCT-3′.

TABLE
Biochemical inhibitory constants for Keap1 inhibitor test set.

| | | | | | Assay 3 | | Assay 4 | |
|---|---|---|---|---|---|---|---|---|
| | Assay 1 | | Assay 2 | | Keap1 | | Keap1 | |
| | WT Keap1 | | WT Keap1 | | (His/GST) | | (His/GST) | |
| | FITC-KL9 | | CDDO-FITC | | CDDO-FITC | | FITC-KL9 | |
| | Cora-1-KL9^{a} | | Cora-1-KL9^{b} | | Tb-Anti-6xHis^{c} | | Tb-Anti-6xHis^{d} | |
| Compound | IC₅₀ | K_{d} | IC₅₀ | K_{d} | IC₅₀ | K_{d, app} | IC₅₀ | K_{d, app} |
| N-KL9 | 2.157 ± | 0.784 ± | 3.468 ± | 1.061 ± | <5% @ | — | 4.881 ± | — |
| | 0.307 | 0.112 | 0.425 | 0.130 | 100 | | 0.324 | |
| Ac-KL9 | 0.126 ± | 0.046 ± | 0.421 ± | 0.129 ± | <5% @ | — | 0.321 ± | — |
| | 0.015 | 0.005 | 0.062 | 0.019 | 100 | | 0.016 | |
| KI-696 | 0.003 ± | 0.001 ± | 0.011 ± | 0.003 ± | <5% @ | — | 0.011 ± | — |
| | 0.001 | 0.000 | 0.002 | 0.001 | 100 | | 0.001 | |
| CDDO | 103.9 ± | 37.78 ± | 0.006 ± | 0.003 ± | 0.004 ± | — | 223.9 ± | — |
| | 33.30 | 12.11 | 0.001 | 0.001 | 0.001 | | 46.20 | |
| CDDO-Me | <10% at | NA | 0.024 ± | 0.013 ± | 0.011 ± | — | 102.6 ± | — |
| | 100 | | 0.003 | 0.002 | 0.001 | | 31.6 | |
| CDDO-JQ1 | <5% at | NA | 3.370 ± | 1.765 ± | 2.681 ± | — | 494.9 ± | — |
| | 100 | | 0.467 | 0.245 | 0.374 | | 261.6 | |
| OBT | 48.91 ± | 17.79 ± | 3.582 ± | 1.876 ± | 3.388 ± | — | 24.61 ± | — |
| | 8.170 | 2.971 | 1.059 | 0.555 | 0.341 | | 1.600 | |
| CS | 30.86 ± | 11.22 ± | 21.84 ± | 11.44 ± | 13.03 ± | — | 16.84 ± | — |
| | 5.280 | 1.920 | 1.970 | 1.032 | 1.350 | | 1.400 | |
| DMF | <5% at | NA | 128.6 ± | 67.36 ± | 64.84 ± | — | 266.4 ± | — |
| | 100 | | 21.40 | 11.21 | 9.330 | | 61.27 | |
| FITC-KL9 | — | 0.004 ± | — | — | — | — | — | 0.002 ± |
| | | 0.001 | | | | | | 0.001 |
| Cora-1-KL9 | — | 0.004 ± | — | — | — | — | — | 0.002 ± |
| | | 0.001 | | | | | | 0.001 |
| CDDO-FITC | — | — | — | 0.164 ± | — | 0.007 ± | — | — |
| | | | | 0.037 | | 0.001 | | |

^{a}5 nM Keap1 (tag-free; 11981-HCNB; Sino Biological), 3.5 nM FITC/Cora-1-KL9 (each), 4 h incubation
^{b}5 nM Keap1 (tag-free; 11981-HCNB; Sino Biological), 5 nM Cora-1-KL9, 150 nM CDDO-FITC, 4 h incubation
^{c}1 nM Keap1 (His/GST; 11981-H20B; Sino Biological), 0.5 nM Tb-Anti-6xHis, 40 nM CDDO-FITC, 4 h incubation
^{d}1 nM Keap1 (His/GST; 11981-H20B; Sino Biological), 0.5 nM Tb-Anti-6xHis, 5 nM FITC-KL9, 4 h incubation
All values are reported in μM and represented as means ± SD
NA = not applicable

TABLE
Biochemical and cellular inhibitory constants for HDAC inhibitor test set.

| | Enzymatic | | TR-FRET assay | | TR-FRET assay | |
|---|---|---|---|---|---|---|
| | activity assay | | HDAC1 | | HDAC1 | |
| | HDAC1 | | Recombinant | | Recombinant | |
| | Recombinant^{a} | | SAHA-NCT^{b} | | M344-FITC^{c} | |
| Compound | IC₅₀ | K_{d} | IC₅₀ | K_{d} | IC₅₀ | K_{d} |
| Panobinostat | 0.003 ± | 0.001 ± | 0.005 ± | 0.002 ± | 0.004 ± | 0.002 ± |
| | 3E−4 | 5E−5 | 5E−4 | 2E−4 | 3E−4 | 1E−4 |
| SAHA | 0.068 ± | 0.017 ± | 0.312 ± | 0.097 ± | 0.185 ± | 0.088 ± |
| | 0.008 | 0.002 | 0.036 | 0.011 | 0.012 | 0.006 |
| CI-994 | 0.459 ± | 0.115 ± | 3.995 ± | 1.241 ± | 2.449 ± | 1.170 ± |
| | 0.057 | 0.014 | 0.425 | 0.132 | 0.223 | 0.106 |
| Cpd-60 | 0.005 ± | 0.001 ± | 0.072 ± | 0.022 ± | 0.043 ± | 0.021 ± |
| | 3E−4 | 8E−5 | 0.007 | 0.002 | 0.003 | 0.001 |
| SABA-NCT | 0.008 ± | 0.002 ± | — | 0.009 ± | — | — |
| | 0.001 | 3E−4 | | 0.001 | | |
| M344-FITC | 0.066 ± | 0.017 ± | — | — | — | 0.064 ± |
| | 0.005 | 0.001 | | | | 0.005 |

| | TR-FRET assay | | TR-FRET assay | | Cellular TR- | | Cellular TR- | |
|---|---|---|---|---|---|---|---|---|
| | HDAC1-HaloTag | | HDAC1-HaloTag | | FRET assay | | FRET assay | |
| | Lysate | | Lysate | | SAHA-NCT | | SAHA-NCT | |
| | SAHA-NCT^{d} | | M344-FITC^{e} | | 0.25 μM^{f} | | 1 μM^{g} | |
| Compound | IC₅₀ | K_{d} | IC₅₀ | K_{d} | EC₅₀ | K_{i, app} | EC₅₀ | K_{i, app} |
| Panobinostat | 0.003 ± | 0.001 ± | 0.002 ± | 0.001 ± | 0.529 ± | 0.018 ± | 1.516 ± | 0.014 ± |
| | 1E−4 | 4E−4 | 2E−4 | 1E−4 | 0.134 | 0.005 | 0.038 | 3E−4 |
| SAHA | 0.111 ± | 0.046 ± | 0.107 ± | 0.054 ± | 42.78 ± | 1.485 ± | 76.14 ± | 0.679 ± |
| | 0.012 | 0.005 | 0.009 | 0.005 | 8.920 | 0.310 | 13.85 | 0.124 |
| CI-994 | 2.346 ± | 0.978 ± | 1.838 ± | 0.919 ± | 106.4 ± | 3.694 ± | <5% @ | NA |
| | 0.171 | 0.071 | 0.112 | 0.056 | 25.54 | 0.887 | 100 | |
| Cpd-60 | 0.054 ± | 0.023 ± | 0.039 ± | 0.020 ± | 18.50 ± | 0.642 ± | 22.81 ± | 0.203 ± |
| | 0.003 | 0.001 | 0.001 | 5E−4 | 3.295 | 0.115 | 4.148 | 0.037 |
| SAHA-NCT | — | 0.009 ± | — | — | — | — | — | — |
| | | 0.001 | | | | | | |
| M344-FITC | — | — | — | 0.075 ± | — | — | — | — |
| | | | | 0.006 | | | | |

^{a}5 nM HDAC1 (His/FLAG; 50051; BPS Biosciences), 18 μM MAZ1600 substrate (3 × K_{M}), 3 h incubation
^{b}5 nM HDAC1 (His/FLAG; 50051; BPS Biosciences), 2.5 nM Tb-Anti-6xHis IgG (AB18184), 20 nM SAHA-NCT, 3 h incubation
^{c}5 nM HDAC1 (His/FLAG; 50051; BPS Biosciences), 2.5 nM Tb-Anti-6xHis IgG (AB18184), 70 nM M344-FITC, 3 h incubation
^{d}1 nM HDAC1-HaloTag (Cora-1-Halo labeled) in HEK293T cell lysate [15 μg/mL], 12.5 nM SAHA-NCT, 3 h incubation
^{e}1 nM HDAC1-HaloTag (Cora-1-Halo labeled) in HEK293T cell lysate [15 μg/mL], 75 nM M344-FITC, 3 h incubation
^{f}HEK293T cells transiently expressing HDAC1-HaloTag, Cora-2-Halo labeled, 0.25 μM SAHA-NCT, 4 h incubation (37° C. and 5% CO₂)
^{g}HEK293T cells transiently expressing HDAC1-HaloTag, Cora-2-Halo labeled, 1 μM SAHA-NCT, 4 h incubation (37° C. and 5% CO₂)
All values are reported in μM and represented as means ± SD
NA = not applicable

### BRIEF SUMMARY

The pace of progress in biomedical research directly depends on techniques that enable the quantitative interrogation of interactions between proteins and other biopolymers, or with their small molecule ligands. Here, time-resolved Förster resonance energy transfer (TR-FRET) assay platforms excel, offering superior sensitivity and specificity. However, the paucity of accessible and biocompatible luminescent lanthanide complexes, which are essential reagents for TR-FRET-based approaches, and their poor cellular permeability has greatly limited broader adaptation of TR-FRET beyond homogenous and extracellular assay applications. In these example, CoraFluors are prepared as FRET donors (which are also useful in the remaining examples herein), a class of macrotricyclic terbium complexes, which are synthetically readily accessible, stable in biological media, and exhibit biologically desirable photophysical and physicochemical properties. This example validates the exceptional performance of CoraFluors in cell-free systems, identify cell-permeable analogs and demonstrate their utility in both quantitative and domain-selective characterization of Keap1 ligands, as well as in live-cell, isoform-selective target engagement profiling as exemplified with HDAC1.

### Introduction

Fluorescence-based technologies are critical for life science research and clinical diagnostics. Most high-throughput assays are based on various modes of fluorescence detection due to their high sensitivity, large-dynamic range, signal stability, variety of readily accessible fluorophores and ease of operation. Similarly, fluorescence-based microscopy techniques comprise arguably the most important imaging technology currently employed in biomedical research. In recent years, the application of time-resolved (TR) fluorescence measurements has greatly improved the sensitivity of homogenous assays and high-resolution microscopy. In particular, the combination of TR readouts with Forster resonance energy transfer (FRET) is highly attractive for studying biological processes on the molecular level. In TR-FRET-based techniques, a signal is generated through FRET between a donor with a long luminescence lifetime and an acceptor fluorophore. The time-gated measurement allows for the virtual elimination of non-specific background originating from scattered excitation light and autofluorescence of screening compounds, biological media, and assay plates; the FRET component limits the readout to acceptor molecules that are in immediate proximity of the donor. This approach therefore enables the quantitative measurement of the interaction of biomolecules and/or small molecule ligands with superior sensitivity and specificity.

Essential for the development of sensitive and robust TR-FRET-based chemogenomic applications is the availability of fluorescence donors that satisfy following criteria: 1) high stability in biological buffers, 2) sufficiently long luminescence lifetime compared to that of autofluorescence generated from biological samples, 3) good quantum yield and brightness, 4) insensitive to assay environment, and 5) scalability and costs. The favorable luminescent properties (long lifetimes. narrow luminescent bands, and large effective Stokes shifts) of lanthanide-based emitters, particularly terbium (Tb) and europium (Eu), make them uniquely attractive as TR-FRET donors. While many luminescent lanthanide complexes have been reported, only a small number are compatible with biological assay conditions. Few of these reagents are commercially available and those that are, are priced at a premium. The precise chemical structures of some of the commercial reagents are not disclosed and published structures generally require lengthy, inflexible, and difficult syntheses, which limits accessibility to these reagents and has hampered broader adaptation, particularly by academic labs. Furthermore, the generally poor membrane permeability renders most of these complexes unsuitable for studying intracellular targets in live cells and therefore has relegated their application primarily to homogenous assay platforms and assays for cell surface receptors.

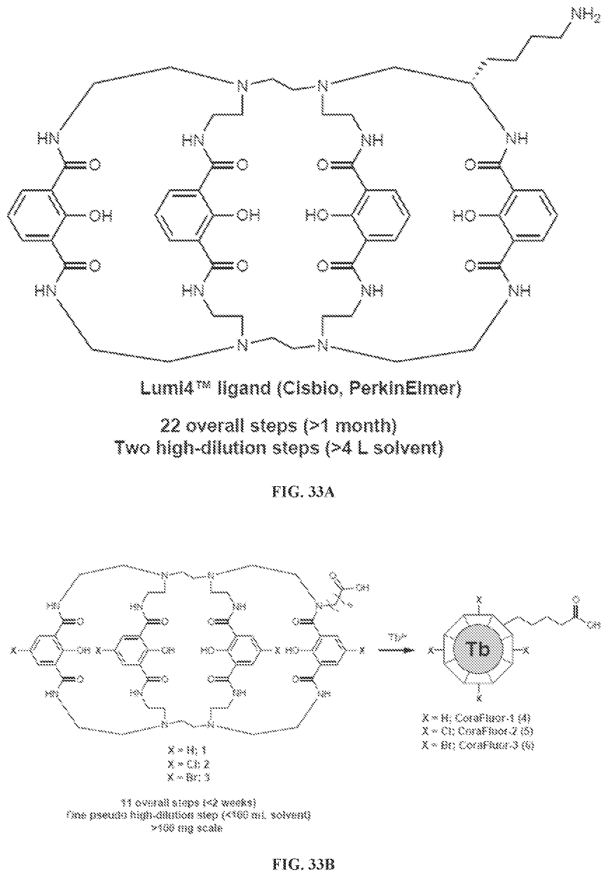

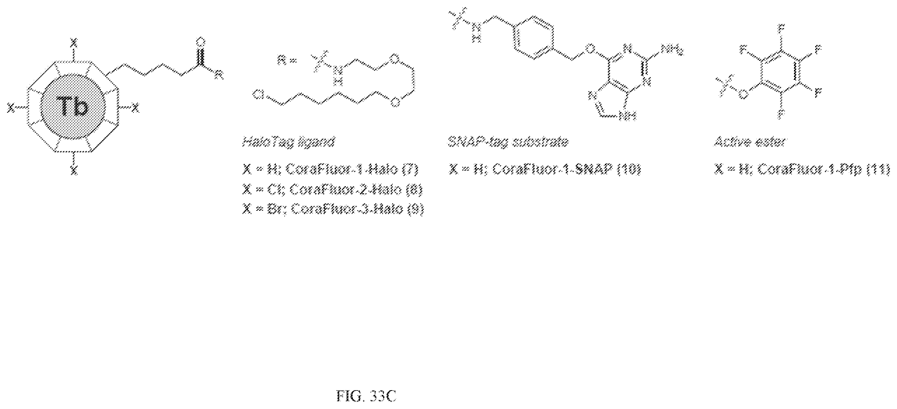

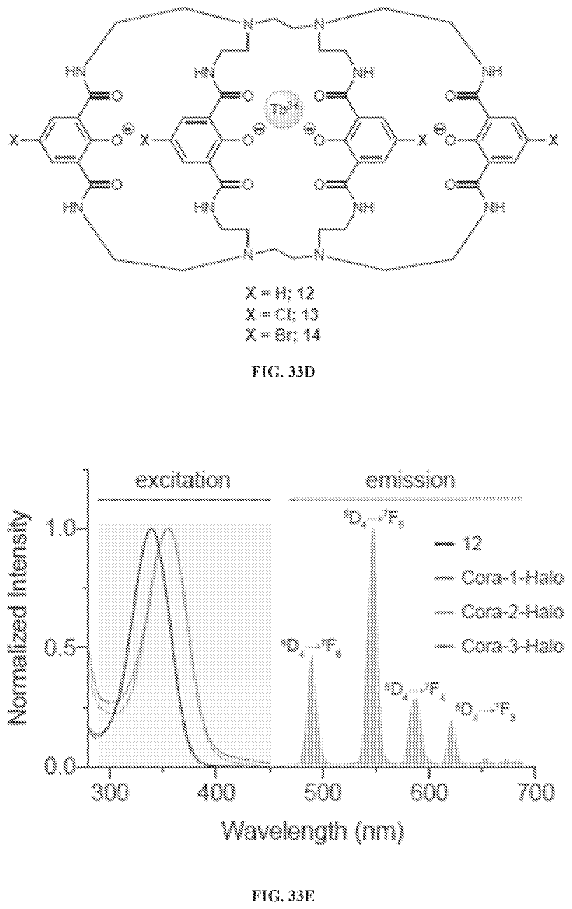

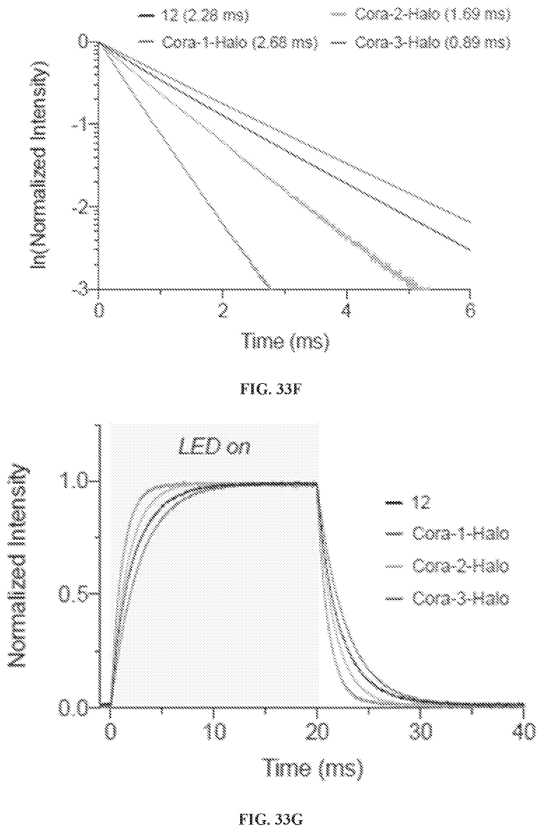

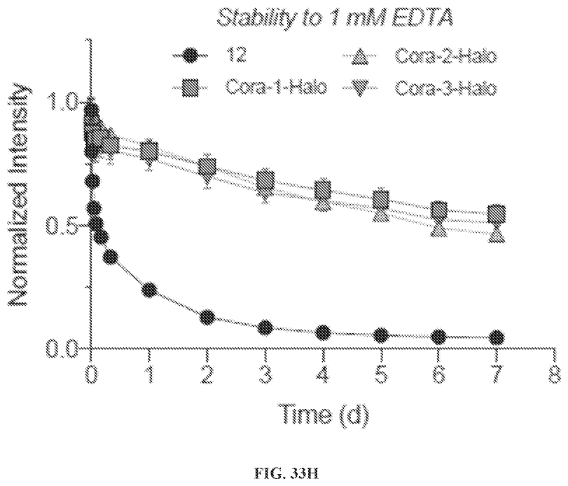

Among the commonly used TR-FRET donors the octadentate tricyclic cryptate Lumi4-Tb™ (FIG. 33a) stands out with several favorable characteristics including good brightness, high quantum yield, long luminescent lifetime, improved stability and broad compatibility with a wide variety of assay conditions compared to other lanthanide complexes. However, high costs (˜$500/μg) and a lengthy, challenging synthesis significantly limits access to this reagent class, especially for experiments that would require milligram quantities for further functionalization. Furthermore, the synthetic strategy poses challenges for the development of analogs with optimized photophysical and physicochemical properties, such as modulation of luminescent lifetimes for multiplexed applications, improved lipophilicity to enable cell permeability, and tuning of the absorbance spectrum to render analogs compatible with common microscopy equipment.

This example reports the development of CoraFluors, a class of macrotricyclic terbium complexes that have been optimized for chemogenomic applications (the CoraFluors are also useful in the remaining examples of this application). CoraFluors offer superior sensitivity and stability in biological settings. The modular synthetic approach provides straightforward access to orthogonally functionalized analogs and allows for rational tuning of the photophysical and physicochemical properties. This example validated the versatility of CoraFluors based on the development of highly specific homogenous assay formats, including in crude lysates, and identified analogs that are cell-permeable. Finally, this example demonstrated that CoraFluors enable quantitative target engagement assays in living cells.

### Results

CoraFluor design and synthetic methodology development: The inherent limitations of Lumi4-Tb™ and other TR-FRET donors have impeded broad accessibility, restricted the ability for chemical modification, and virtually precluded intracellular applications. We therefore set out to develop luminescent lanthanide complexes that are compatible with a wide variety of biological conditions and are suitable for live-cell assay applications, including targeted delivery to intracellular proteins.

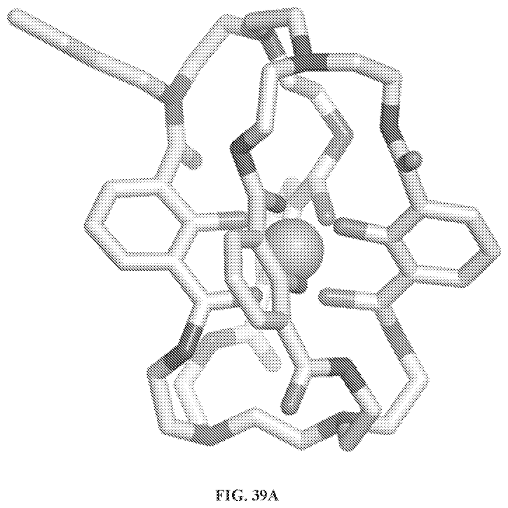

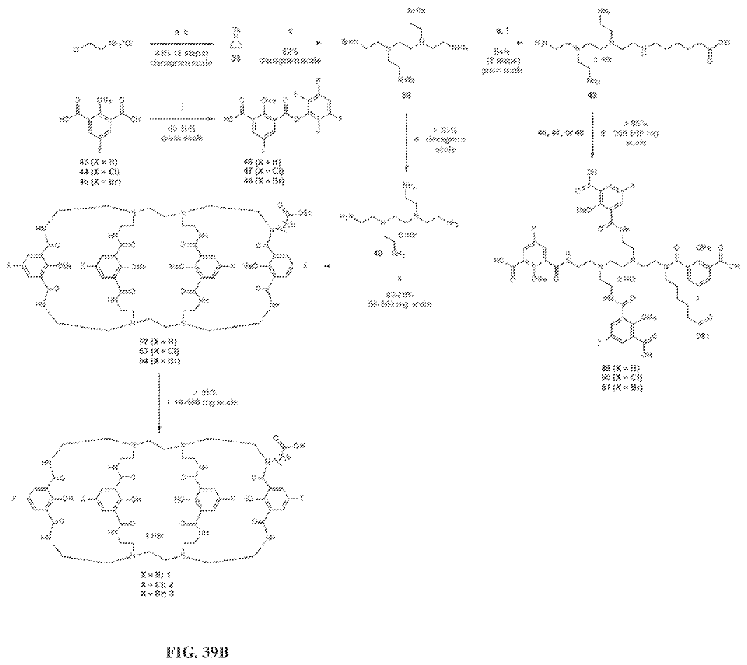

When this example evaluated a 3D-model of the Lumi4-Tb™ complex it was noticed that, in contrast to crystal structure for the protected macrocyclic precursor, four of the eight amides are oriented with the N-H hydrogens pointing outward (FIG. 39A). We hypothesized that the previously unexplored alkylation of one of the amide nitrogens would be tolerated and potentially even stabilize the complex, providing an alternative linker attachment point. This strategy, combined with additional methodological changes, considerably simplified the synthesis, offering access to the carboxylinker-modified macrocyclic ligand (1, FIG. 33b) in just 11 overall steps (outlined in FIG. 39), compared to 22-steps reported for Lumi4-Tb™ (38A)²⁰. Furthermore, our approach also enabled facile access to novel core-substituted analogs, including the chlorinated (2) and brominated (3) derivatives, allowing for systematic modulation of the physical properties of the final terbium complexes (CoraFluor-1/2/3 (4-6); FIG. 33b).

The free carboxylate can be further functionalized to produce, as examples, HaloTag (HT), SNAP-tag, and active esters such as pentafluorophenyl (Pfp)—CoraFluor-1/2/3-Halo (7-9, Cora-1/2/3-Halo), CoraFluor-1-SNAP (10, Cora-1-SNAP), and CoraFluor-1-Pfp (11, Cora-1-Pfp), respectively (FIG. 33c). The direct labeling of proteins through amine-acylation is often the preferred approach for chemical tagging using active esters. However, this is challenging for lanthanide complexes containing multiple chelating carboxylates. While the preparation of the corresponding NHS-esters of CoraFluors is straightforward, we found the Pfp-ester to be preferable for protein labeling. Following the same strategy we also synthesized analogs with modified isophthalamides to access the linker-less reference complexes (X═H, (12); Cl (13); Br (14); FIG. 33d) to serve as comparisons to Lumi4-Tb™ in our studies.

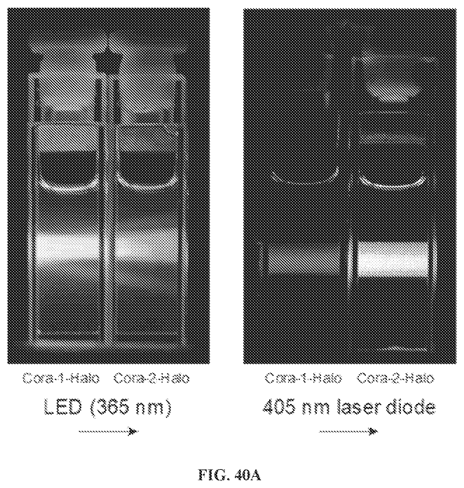

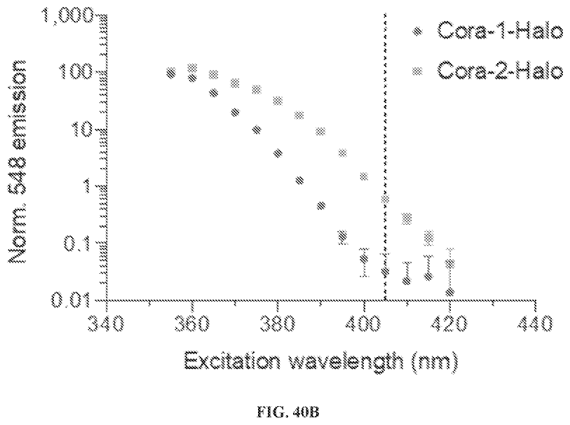

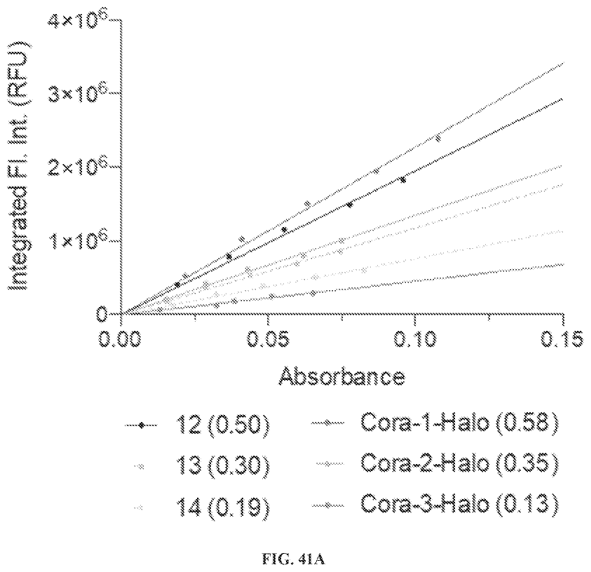

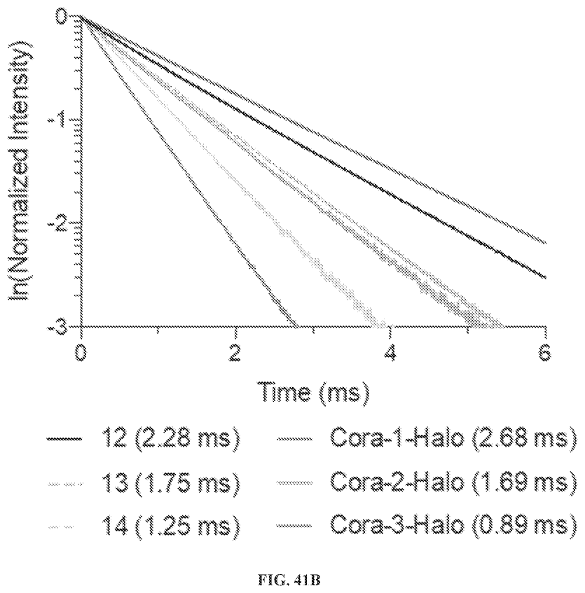

CoraFluors exhibit desirable photophysical and physicochemical characteristics: we first determined the photophysical properties of CoraFluors-1/2/3 relative to the linker-less complexes 12-14. The emission spectra of all compounds (example Cora-1-Halo; FIG. 33e) displayed the characteristic line-like Tb emission bands arising from the ⁵D₄→⁷F_{J} transitions of the lanthanide center. Notably, the absorbance maxima of the halogenated derivatives exhibited a bathochromic shift of ˜15-16 nm relative to the unsubstituted complexes that are centered about 340 nm, therefore enabling a more efficient excitation at 365 nm and even extending to 405 nm, two channels that are more commonly found in imaging settings (FIG. 22e, FIG. 40, Table). The CoraFluor complexes possess quantum yields and luminescence lifetimes ranging from 0.13 to 0.58 and 0.89 to 2.68 ms, respectively (FIG. 33f, FIG. 41, Table). Cora-2-Halo and Cora-3-Halo showed faster excitation kinetics when compared to Cora-1-Halo (FIG. 33g). When using pulsed lasers or high-powered xenon-flash lamps, which can deliver high excitation light energy within <1 μs, near quantitative excitation can be accomplished even for complexes with slow excitation rate constants (kₑₓ). However, when using other illumination methods, such as incandescent light sources or LEDs, which are commonly used in microscopy setups or low-end spectrophotometers and plate readers, the signal intensity will directly depend on kₑₓ. Importantly. the CoraFluor derivatives Cora-2-Halo and Cora-3-Halo were more lipophilic and exhibited log D values close to 1-3, a range that is generally considered optimal for cell membrane permeability (Table).

Introduction of the tertiary amide linker was not detrimental but rather improved the photophysical properties of most complexes, and we observed a universally beneficial effect when we assessed CoraFluor stability towards commonly used biological buffers compared to their linker-less counterparts (FIG. 33h-i, FIG. 38). CoraFluors are compatible with a wide range of biologically relevant buffer conditions over several days, including strong chelators such as reducing agents, acidic pH, and bivalent metal ions including Cu²⁺ and Mn²⁺, which are not tolerated by most other TR-FRET donor ligands. The CoraFluor complexes also displayed much higher kinetic stability to chelators such as ethylenediaminetetraacetic acid (EDTA) and diethylenetriaminepentaacetic acid (DTPA), which we hypothesize is due to the rigidification of the ligand scaffold by the tertiary amide linker.

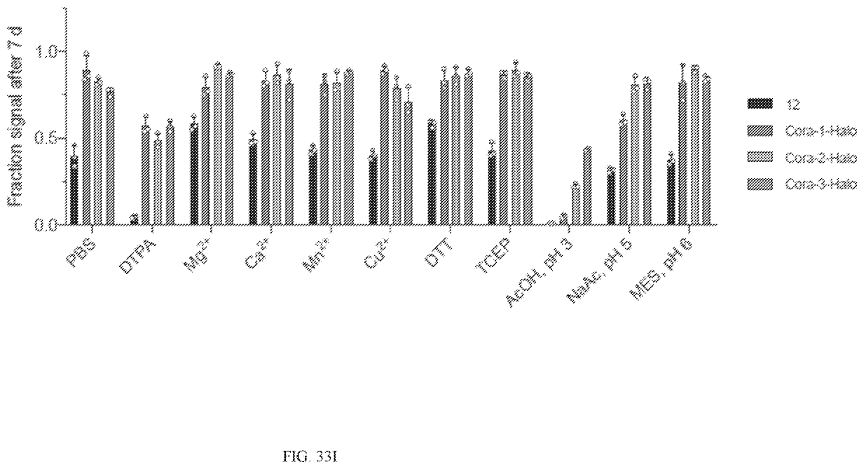

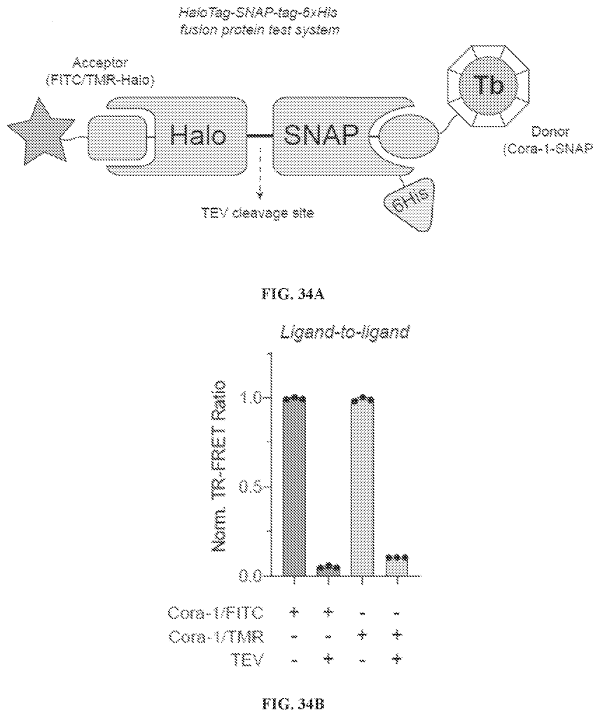

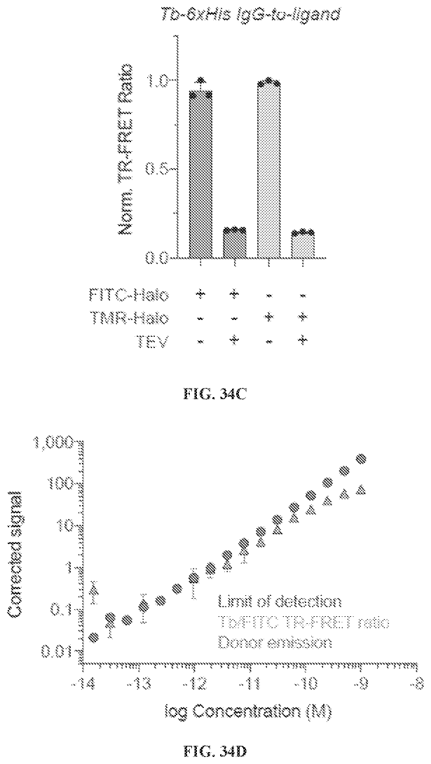

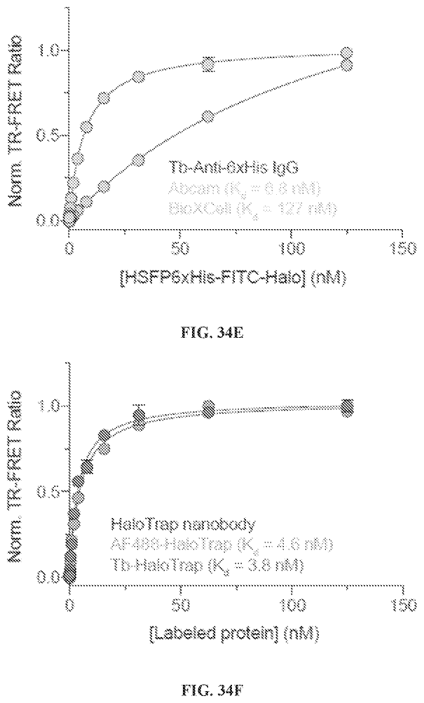

CoraFluors demonstrate excellent performance in homogenous biochemical assays: to evaluate CoraFluors in cell-free biochemical assays, we developed a robust benchmarking platform that uses self-labeling protein tags. Specifically, this example cloned and expressed an engineered HaloTag-SNAP-tag fusion protein containing a C-terminal hexa-histidine tag (HSFP6×His, FIG. 34a). The modular design enables the orthogonal labeling of individual domains with small molecule probes (e.g. fluorophores, affinity handles) in defined stoichiometries, resembling ligand binding events and/or protein-protein interactions without potentially confounding effects due to ligand dissociation. Both Cora-1-Halo and Cora-1-SNAP efficiently reacted with their respective fusion protein partner (38G). Furthermore, specific TR-FRET signal was observed in orthogonal experiments employing either Cora-1-SNAP or CoraFluor-1 labeled anti-6×His IgG, with both HaloTag-ligand functionalized FITC (15, FITC-Halo) and TMR (16, TMR-Halo) as fluorescent acceptors, where signal could be depleted upon addition of TEV protease (FIG. 34b-c, FIG. 38H). To gauge the limit of detection of Cora-1-SNAP in combination with FITC-Halo, we performed a dose-titration experiment which showed that specific signal was robustly quantified at sub-picomolar concentrations using a monochromator-based plate reader (FIG. 34d).

Because of the high costs and/or limited availability of lanthanide complexes that are suitable for direct protein labeling, indirect methods are generally used to install TR-FRET donors on a given protein of interest (e.g. antibodies or biotin/streptavidin). However, direct labeling of the target can be desirable as it allows for better-defined stoichiometry of ligand-target complexes, simplifying the quantitative determination of biochemical constants. For example, when using a monoclonal IgG antibody against a single epitope, two equivalent binding sites (or four in the case of streptavidin) are formed. Occupation of either site with an acceptor-labeled tracer will generally result in maximum TR-FRET signal, which needs to be accounted for in the regression analysis. Polyclonal antibodies can result in higher order complexes, which may enable more sensitive detection, but are difficult to model quantitatively. Regardless, care must be taken that the antibody exhibits sufficiently tight binding to its antigen. To this extent we used a pairwise labeling strategy to compare two commercial anti-6×His antibodies (Abeam and BioXCell) using our HSFP6×His profiling platform and found substantial differences between the Abeam (K_{d}=6.8±0.4 nM) and BioXCell antibody (K_{d}=127+7 nM) (FIG. 34e). Similarly, we used Cora-1-Pfp to label an anti-HaloTag nanobody (HaloTrap: ChromoTek), which binds HSFP6×His even more tightly (K_{d}=3.8±0.4 nM, FIG. 34f). Since nanobodies consist of a single monomeric variable antibody domain they cannot dimerize or oligomerize the target protein, retaining the original target stoichiometry and therefore might serve as useful tools in TR-FRET assay development.

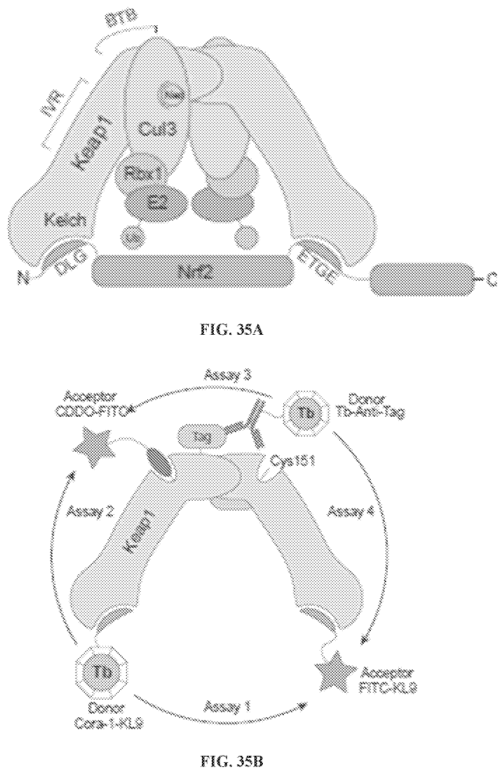

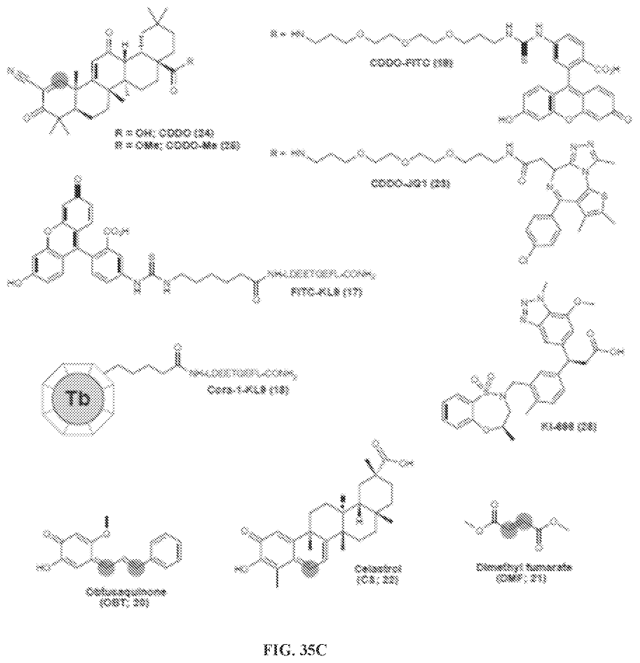

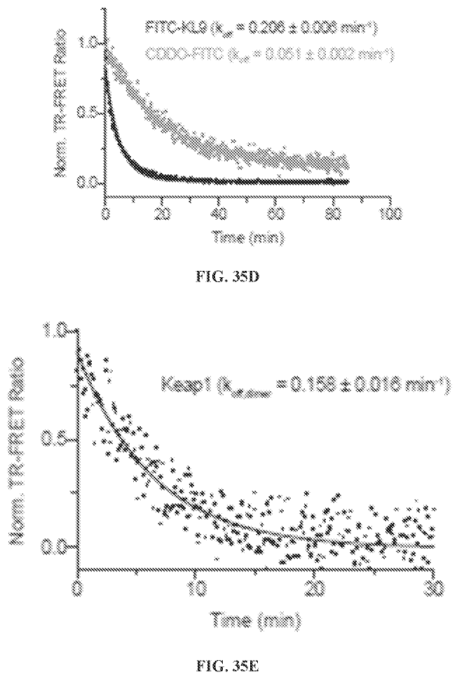

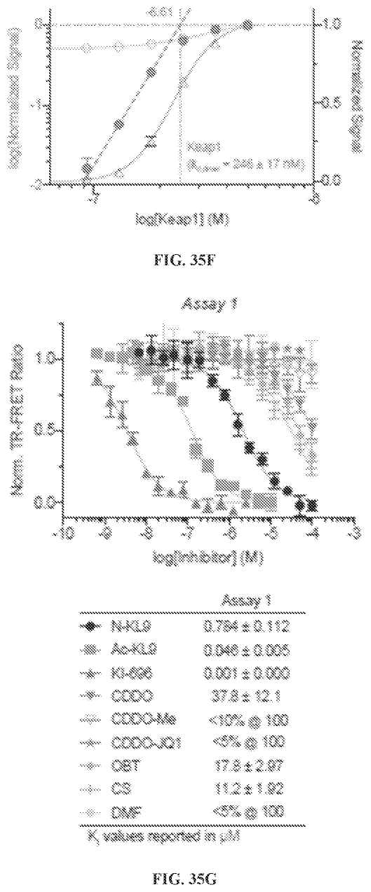

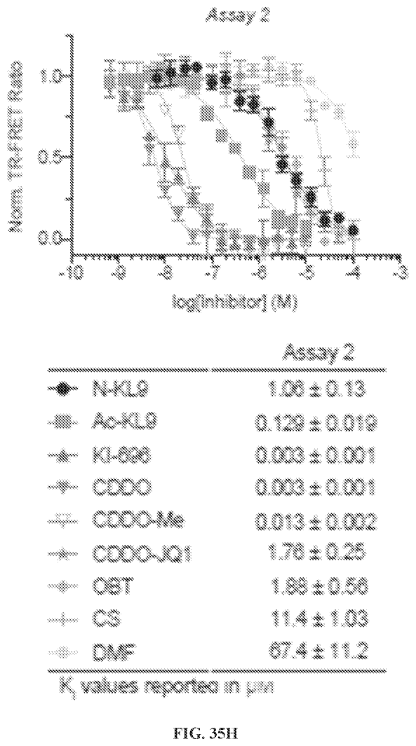

CoraFluors enable domain-specific and quantitative characterization of Keap1 binders: we next set out to employ CoraFluors for the development of a multimodal biochemical TR-FRET assay platform for Keap1 (Kelch-like ECH-associated protein L: FIG. 35a-b). Keap1 is redox-regulated member of the CRL3 (Cullin-RING E3 ligase) complex and forms a homodimer via its N-terminal BTB domain, which also contributes to the binding of Cul3 (Cullin 3). The C-terminal Kelch domains of the homodimer function as a substrate adaptor for the transcription factor Nrf2 (Nuclear factor erythroid 2-related factor 2). During cellular homeostasis. Keap1 recruits Nrf2 and thereby promotes its ubiquitination and subsequent proteasomal degradation. This process is disrupted by oxidative stress or by small molecules, including thiophilic compounds, that can covalently bind to cysteine-rich Keap1, resulting in stabilization and subsequent nuclear translocation of Nrf2, where it regulates the expression of antioxidant response element (ARE) dependent genes.

In recent years, pharmacological targeting of Keap1 with small molecules for the treatment of neurodegenerative, inflammatory, and malignant disorders has received increasing attention. More recently, recruitment of neosubstrates to the Keap1/CRL3 complex for targeted protein degradation via heterobifunctional degraders has been reported. Most rational ligand design, in particular for non-covalent inhibitors, has focused on the development of Kelch domain binders to directly block Nrf2 recruitment. In contrast, electrophilic compounds have been proposed to exert their activity mostly through binding to the BTB domain, targeting the regulatory Cys-151 residue. However, the lack of robust assays, specifically for reversible covalent ligands, that readily enable the profiling of both binding affinities and kinetics has hampered inhibitor development and has limited mechanistic understanding of Keap1. Only very recently TR-FRET assays based on recombinant, epitope-tagged, truncated Keap1 have been developed. Although these strategies were shown to greatly improve sensitivity over other assay technologies, they were limited to interrogating the Kelch domain and did not offer the ability probe other, more elusive aspects of Keap1.

The nonspecific binding of covalent ligands to reporter antibodies can result in measurement artifacts that are difficult to control for. We hypothesized that the pairwise combination of orthogonally donor/acceptor labeled small molecule ligands would circumvent these potential limitations and pitfalls. By eliminating the requirement for antibodies, streamlined TR-FRET assays, and even previously intangible experiments, are readily enabled with tag-free, full-length, wild-type Keap1 (FIG. 35b). In addition to a fluorescently-labeled 9-mer peptide (17, FITC-KL9) derived from residues 76-84 of Nrf2 (LDEETGEFL), we synthesized the analogous CoraFluor labeled peptide (18, Cora-1-KL9) and also developed a BTB domain-specific tracer based on the semi-synthetic triterpenoid bardoxolone (19, CDDO-FITC; FIG. 35c). CDDO has been shown qualitatively to bind with high affinity via formation of a reversible covalent adduct with Cys-151.

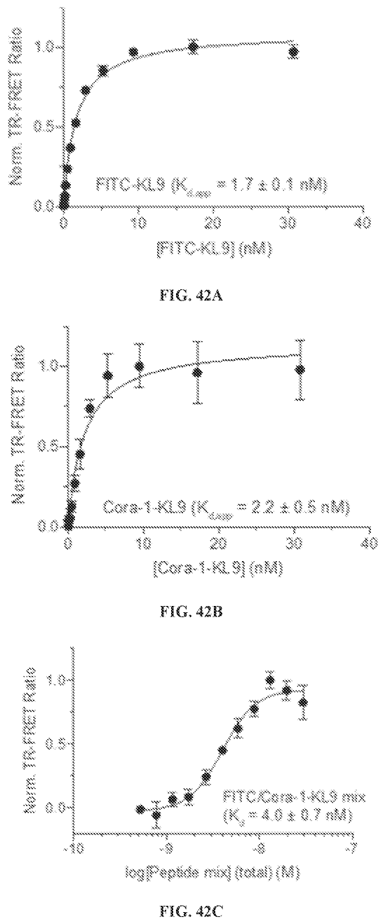

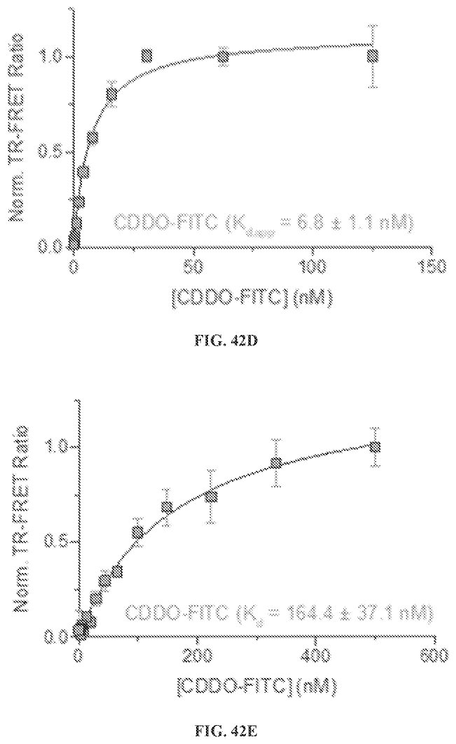

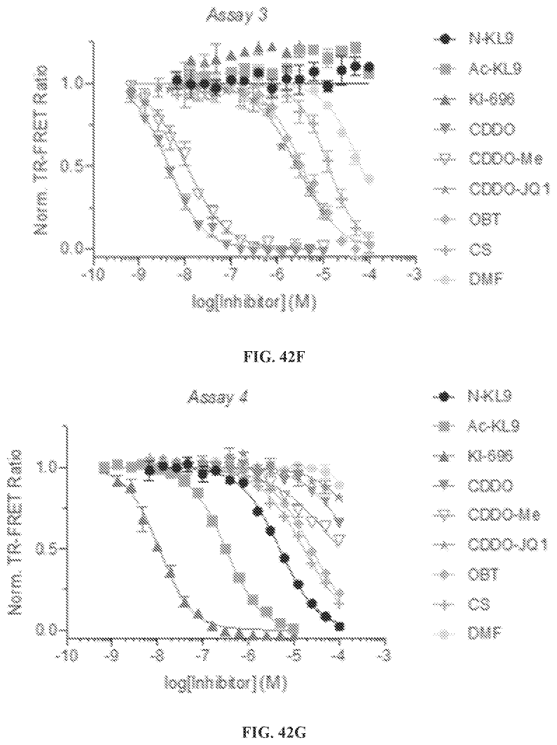

This example first validated the FITC-KL9 and CDDO-FITC tracers. Employing our CoraFluor-labeled anti-6×His antibody (see above) and recombinant, full-length Keap I with N-terminal 6×His/GST tags, dose titration of the respective tracer ligands yielded apparent equilibrium dissociation constants (K_{d,app}) of 1.7±0.1 and 6.8±1.1 nM for FITC-KL9 and CDDO-FITC, respectively (FIG. 42). While we are not aware of any applicable reference data for CDDO, our determined K_{d,app} value for FITC-KL9 was comparable to literature values of similar tracers. Measurement of the first-order dissociation rate constant (k_{off}) by ligand displacement yielded k_{off} values of 0.206±0.006 min⁻¹ and 0.051 t 0.002 min⁻¹ (FIG. 35d), establishing a minimum incubation time of ˜15 min and ˜60 min for equilibrium conditions (5 half-lives) for FITC-KL9 and CDDO-FITC, respectively.

Next, the example performed a saturation binding experiment using an equimolar mixture of FITC-KL9/Cora-1-KL9 and wildtype, full-length Keap1, which we reasoned would exist to some fraction as a homodimer in solution and therefore result in productive complexes with an acceptor and donor ligand bound. Indeed, we observed a dose-dependent increase that matched a two-site binding model and yielded a K_{d}-value of 4.0±0.7 nM (42c). Furthermore, rapid dilution of Keap1 at constant ligand concentrations allowed facile determination of the dimer dissociation rate constant k_{off,dimer}=0.158±0.016 min⁻¹ (FIG. 35e). Similarly, when profiling Keap1 in dose response against an equimolar mixture of FITC-KL9/Cora-1-KL9 at saturation binding concentrations (300 nM each), we determined the equilibrium dissociation constant of the Keap1 dimer to be K_{d,dimer}=246±17 nM (FIG. 35f). Last, we performed a saturation binding experiment with CDDO-FITC at fixed concentrations of Cora-1-KL9 and Keap l to establish a dissociation binding constant for CDDO-FITC. Since monomeric Keap1 will be the dominant species at low nanomolar concentrations we fit a one-site binding model, yielding a K_{d}=164.4±37.1 nM (42e).

To establish robust assay parameters, we optimized the ligand and protein concentrations and found that 3.5 nM of each KL9 tracer (FITC-KL9. Cora-1-KL9) and 5 nM wildtype Keap1 (Assay-1) provided excellent assay performance (Z′=0.71), while 5 nM Cora-1-KL9, 150 nM CDDO-FITC and 5 nM wildtype Keap1 (Assay-2) exhibited similar robustness (Z′=0.73). Assay-2 is unique and offers the distinct advantage to simultaneously probe two-binding sites, with the signal originating from the domain for which the corresponding small molecule exhibits the higher affinity, while Assay-1 can potentially identify ligands that disrupt or stabilize Keap1 dimers.

This example first used these assay systems to profile the relative affinities and domain-selectivity of a series of thiophilic small molecules, including the cysteine-reactive para-quinone methide obtusaquinone (20. OBT), which we have recently shown to be a reversible covalent thiophile that targets Keap1 and induces its degradation, dimethyl fumarate (21, Tecfidera), the pentacyclic triterpenoid celastrol (22, CS) that is known to form reversible covalent thiol-adducts, and finally a CDDO-derived heterobifunctional degrader (23, CDDO-JQ1), for which a close analog has been reported recently, to establish evidence for direct Keap1 engagement and to provide a comparison to CDDO (24) and bardoxolone methyl (25, CDDO-Me), which is currently undergoing Phase 3 clinical trials (NCT03550443) for diabetic kidney disease. Our inhibitor set furthermore included the acetylated (26, Ac-KL9) and free N-terminal (27, N-KL9) LDEETGEFL peptides, as well as the potent small molecule Kelch ligand KI-696 (28), all of which are noncovalent ligands (FIG. 35c).

As expected, the three Kelch-targeted ligands were the most potent inhibitors in Assay-1, accurately reflecting their reported affinities (FIG. 35g, i: Table). Surprisingly, CDDO, OBT and celastrol also exhibited micromolar activity in this assay, while CDDO-Me, CDDO-JQ1 and dimethyl fumarate did not show any appreciable affinity below 100 μM. These results can be rationalized by the presence of a cysteine residue (Cys-434) in the Nrf2 binding site, which, if sterically permitted, can react with the more thiophilic ligands. This feature can be exploited for rational ligand design.

Conversely, when this example tested Keap1 informer set in Assay-2. the Kelch ligands exhibited virtually identical activities as in Assay-1 (FIG. 35h-i). These results also confirm that the activity observed in Assay-1 is not the result of ligand-induced disruption of the Keap1 homodimer. Additionally. all thiol-reactive ligands were active in this assay. CDDO and CDDO-Me were the most potent competitors of CDDO-FITC, with the other cysteine-reactive compounds displaying significantly lower albeit differential potencies. However, the heterobifunctional degrader CDDO-JQ1 was >500-fold less potent than CDDO and comparable to the affinity of CDDO-FITC. These findings highlight the importance of optimizing the linker attachment strategy when employing CDDO as an E3-targeting warhead for degrader development and, based on the BTB-CDDO co-crystal structure (PDB: 4CXT), suggest that some of CCDO's methyl groups, while synthetically more challenging, might provide better vectors for linker attachment when developing degraders with high selectivity for Keap1. Interestingly, celastrol, which displayed the highest affinity in Assay-1, was only slightly more potent in Assay-2, suggesting it has comparable affinity to both Kelch and BTB domains (also see FIG. 42). To validate the BTB domain selectivity of the inhibitor set, we also performed a single ligand-displacement assay using 6×His/GST-Keap1 in combination with CoraFluor-labeled anti-6×His antibody and CDDO-FITC (Assay-3), which further confirmed the activity of OBT and celastrol, and proved that Ac-KL9. N-KL9, and KI-696 are highly specific for the Kelch-domain (42f-g).

CoraFluors are cell permeable and enable target engagement studies in live cells: The ability to interrogate the interaction of small molecule ligands with their target proteins in living cells with spatiotemporal resolution is of great interest in biomedical research. Only recently we and others have developed suitable technology platforms based on fluorescence polarization microscopy or bioluminescence resonance energy transfer (BRET) that enable such studies. TR-FRET holds great promise in combining the strengths of both platforms to offer high sensitivity, tight spatial control and high-throughput. However, to the best of our knowledge, no generally suitable TR-FRET donors for intracellular application have been reported to date. While Lumi4™ has been used in live cells, those strategies have required conjugation to cell penetrating peptides or harsh conditions such as microinjections or electroporation, which preclude a more generalized experimental approach.

To evaluate the potential of CoraFluors for intracellular application we first tested their ability to label EGFP-HT in HEK293T cells. Consistent with the improved lipophilicity of the chlorinated cryptates we found that Cora-2-Halo (log D=0.7) but not Cora-1-Halo (log D=−0.7) efficiently labeled intracellular EGFP-HT in a dose-dependent manner (FIG. 43). These results provide further support that the observed TMR-Halo competition with Cora-2-Halo is not generated from excreted EGFP-HT or membrane-compromised cells. Furthermore, we were able to measure specific TR-FRET signal between Cora-2-Halo and EGFP that was competed by pre-treatment with a non-fluorescent HaloTag ligand (29, Ac-Halo) (FIG. 43b).

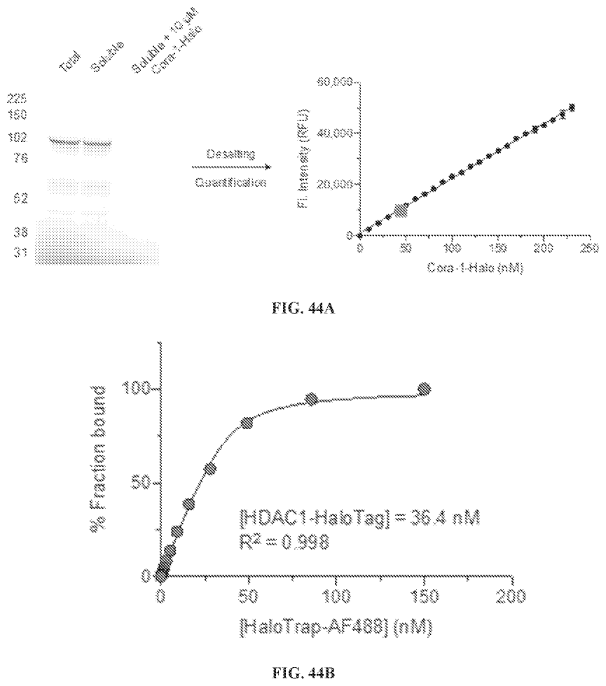

Encouraged by these findings we sought to apply our system to quantify target engagement of small molecule ligands. We selected HDAC1 (histone deacetylase 1) as a model system, which has previously been used for the development of intracellular BRET target engagement assays for HDAC inhibitors. We cloned HDAC1 into a C-terminal HaloTag expression vector and optimized transient transfection of the construct in HEK293T cells. Transfection and expression efficiency of HDAC1-HT was validated via treatment with TMR-Halo and fluorescence microscopy (381). To better quantify the intracellular expression levels of HDAC1-HT, we analyzed lysates of TMR-Halo labeled HDAC1 by SDS-PAGE, followed by imaging with a fluorescence gel scanner (FIG. 44), which provided an estimated concentration in the mid-micromolar range.

Although fluorescence gel imaging is widely applied for protein quantification, the throughput is limited, and most labs are not equipped with suitable laser gel imagers due to their high costs, which often leaves a lengthier Western Blot analysis as the default option. A more facile and less expensive method that would enable the quantification of HaloTag (or other) fusion proteins directly in lysates would therefore be highly desirable. We rationalized that the pairwise combination of a HaloTag-ligand and HaloTrap nanobody could enable a homogenous assay platform suitable for high-throughput. Using an AlexaFluor488-labeled HaloTrap nanobody (AF488-HaloTrap, see above) in combination with Cora-1-Halo, we determined the lysate concentration of HDAC1-HT to be 400 nM, which is well in agreement with our results obtained from gel and fluorescence-based quantification (FIG. 44b).

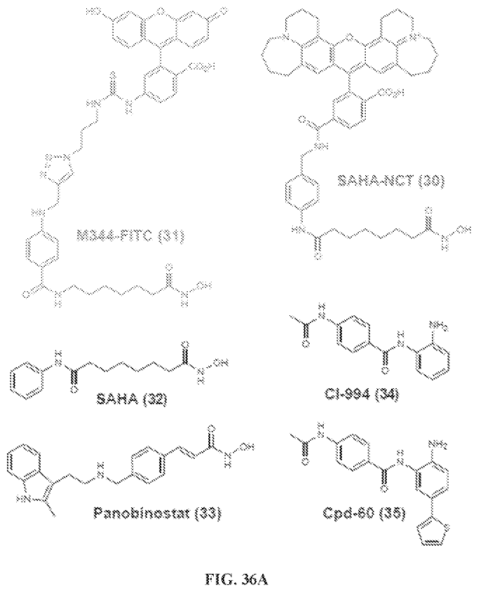

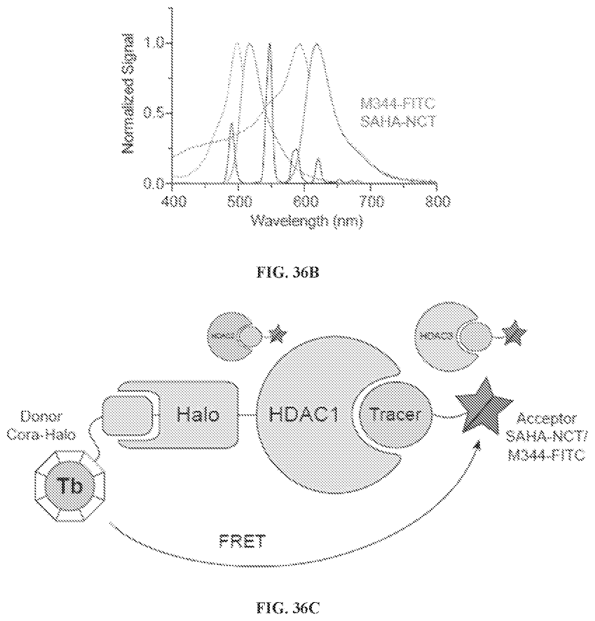

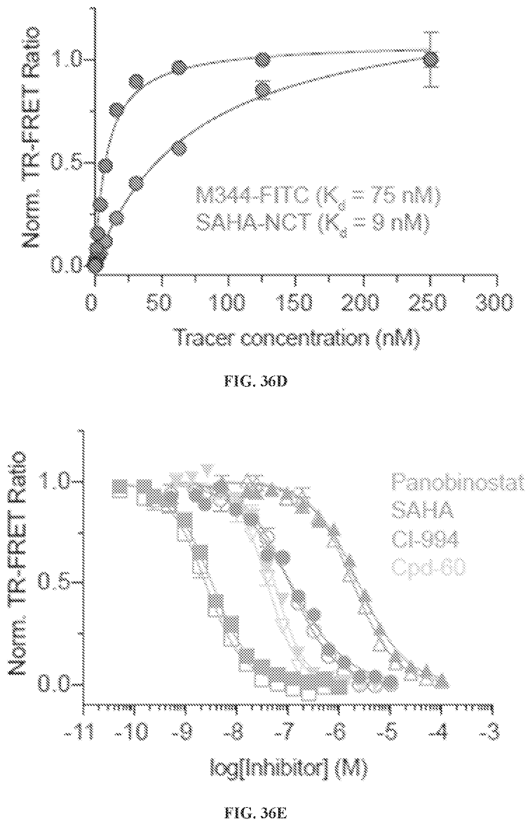

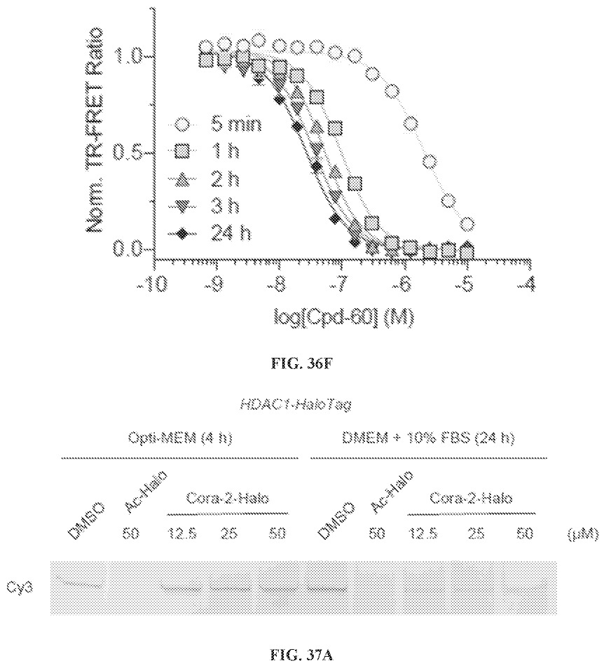

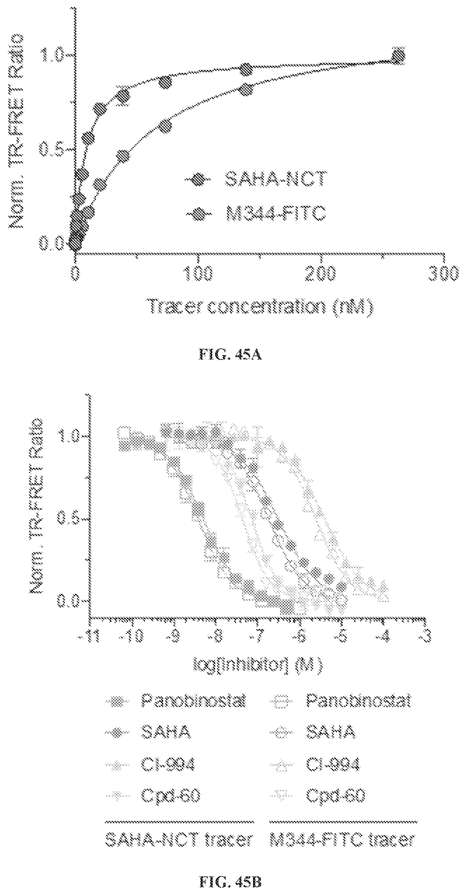

Next, this example determined the equilibrium binding constants of the fluorescent HDAC inhibitors SAHA-NCT and M344-FITC (30 and 31, respectively: FIG. 36a) for recombinant HDAC1 under cell-free conditions to validate their applicability as TR-FRET tracers. Both tracers provided good spectral overlap between donor emission and acceptor absorbance (FIG. 36b). SAHA-NCT has been reported as an optimized probe for cellular HDAC target engagement studies using NanoBRET and was selected to allow for better comparison between the two platforms. M344-FITC has previously been developed by our group as ligand for fluorescence polarization-based biochemical HDAC assays and was included as a cell-impermeable control compound. The IC₅₀/K_{d} values obtained with our representative inhibitor set (SAHA, 32; panobinostat, 33; CI-994, 34; Cpd-60, 35; FIG. 36a) using recombinant, purified protein were consistent with related inhibitors as determined in homogenous TR-FRET and HDAC activity assays using the same construct (FIG. 45, Table). Notably, a distinct advantage of TR-FRET assays over traditional enzyme activity and fluorescence polarization-based assays is their compatibility with lysates, enabling the selective interrogation of a specific HDAC target in the direct presence of other HDAC isoforms (FIG. 36c). Taking advantage of the highly specific nature of the HaloTag we used Cora-1-Halo labeled lysate from transient expression of HEK293T cells for the characterization of HDAC tracers and inhibitors. This strategy readily allowed us to determine Ki values and monitor the binding kinetics for slow-binding inhibitors, as exemplified with the well-characterized Cpd-60—all without the need for further purification or enrichment of the target protein (FIG. 36c-f).

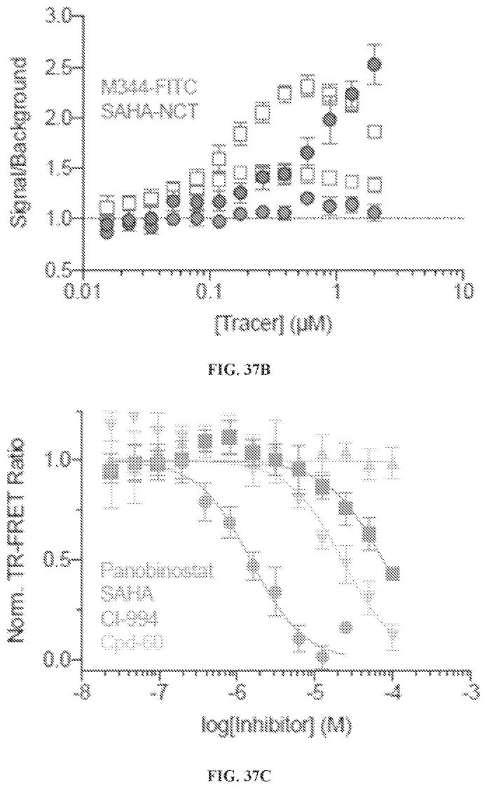

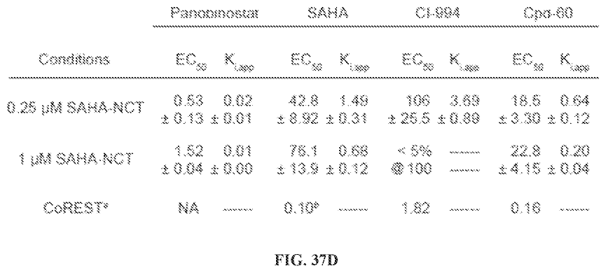

Having demonstrated the robustness and versatility of our methodology in a cell-free system we next sought to validate the approach for intracellular target engagement studies. Following further optimization of our labeling protocol, we achieved efficient (>70%) intracellular Tb-tagging of HDAC1-HT using cell culture medium supplemented with Cora-2-Halo at 12.5 μM (FIG. 37a, FIG. 38J). As shown in FIG. 37b, in live cells we were able to measure specific and ligand concentration-dependent TR-FRET signal for SAHA-NCT but not M344-FITC, consistent with the poor cell-permeability of M344-FITC. However, concentration-dependent TR-FRET signal was detected for both tracer compounds after addition of lysis buffer and the compromising of cellular membranes, further validating that the observed signal is derived from uncompromised cells. In all cases the specific TR-FRET signal was abolished by addition of excess panobinostat.

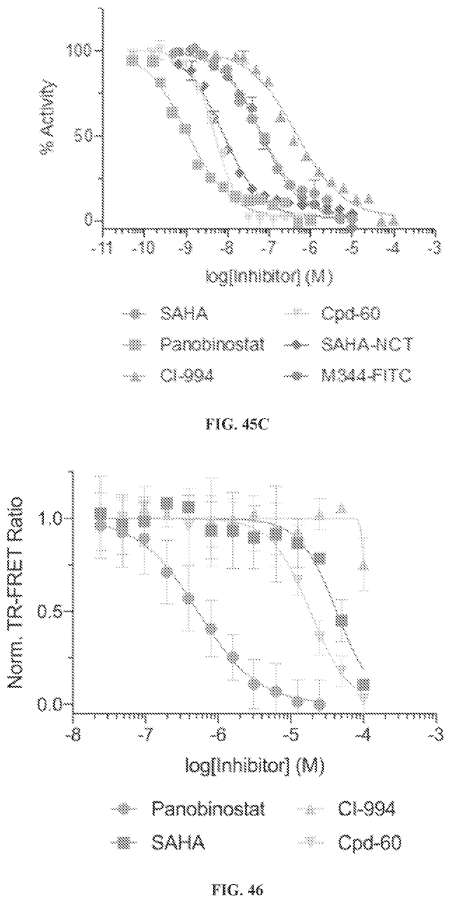

Finally, we evaluated the utility of our TR-FRET platform for the profiling of intracellular target engagement of our representative inhibitor set with HDAC1. We incubated HDAC1-HT-expressing, Cora-2-Halo labeled HEK293T cells with 1 μM SAHA-NCT and measured TR-FRET signal in the absence and presence of varying concentrations of HDAC inhibitors. As shown in FIG. 37c-d, we were able to observe dose-dependent decrease in signal for the inhibitors, recapitulating the relative potencies determined in cell-free and biochemical assays. Importantly, lower concentrations of SAHA-NCT resulted in the expected relative decrease of cellular EC5o values (FIG. 37d, FIG. 46). Correction of the cellular EC₅₀ values according to Cheng-Prusoff provided absolute constants that closely matched both our cell-free (lysate) values, as well as literature Ki values obtained in biochemical activity assays using the immunoaffinity-purified dominant nuclear HDAC1 corepressor complex (FIG. 37d, Table).

### Discussion

TR-FRET synergistically combines superior sensitivity with exquisite specificity, offering many distinct advantages over other assay modalities commonly employed in biomedical research. However, the lack of affordable, readily available, and high-performance small molecule probes that are suitable as donor fluorophores for TR-FRET experiments, which are compatible with a wide range of biological conditions and are cell permeable, has precluded a broader adaptation and incorporation of this methodology into the chemical biology toolbox as a default assay platform.

CoraFluors address most of the existing shortcomings of current TR-FRET donors and will facilitate previously elusive experimental approaches. Importantly, CoraFluors arc readily compatible with most small molecule tracers of existing FRET, BRET and FP platforms. enabling seamless integration while providing superior assay performance. CoraFluors are easily accessible via a concise, robust, modular, and scalable synthesis that is compatible with late-stage functionalization toward almost any conceivable bio-orthogonal handle, including amine-reactive esters, which arc more challenging to prepare with aminocarboxylate-derived chelators. Furthermore, their compatibility and stability characteristics in biological media outperform commercially marketed products.

This example has exemplified the versatility and robustness of CoraFluors by developing a novel and sensitive TR-FRET assay platform for Keap1 that allows for comprehensive and site-specific characterization of ligands using full-length, untagged protein. Our strategy eliminates the requirement for antibodies or streptavidin-based techniques for the installation of the TR-FRET donor, which greatly reduces the complexity of the system, and enables the precise measurement of binding affinities and kinetic parameters not only for BTB-targeted cysteine reactive small molecules, but also for interactions between Keap1 monomers, all of which have not been possible before. Together, this has provided important mechanistic insights in the activity of Keap1 inhibitors and will support the discovery and development of next generation ligands. Importantly, similar strategies will be readily adaptable to other proteins and biomolecules for the detailed characterization of small molecules, including reversible covalent ligands.

Most notably, this example demonstrated that CoraFluors enable the quantitative measurement of small molecule target engagement in living mammalian cells. The ability to measure the interaction of small molecules with their cellular targets is critical to establishing a comprehensive understanding of drug action. In this respect, the cellular thermal shift assay (CETSA) provides a straightforward approach to not only establish experimental support for specific ligand-protein interactions, but also to identify secondary protein targets of a small molecule of interest. However. CETSA is a destructive end-point assay and does not allow for the direct, quantitative and kinetic measurement of drug binding, but rather requires cell lysis and consecutive analysis by other analytical platforms (e.g. ELISA or quantitative proteomics). We have previously established a fluorescence polarization microscopy approach to address this unmet need. More recently, BRET-based methods have been developed. In particular the development of small, efficient, and stable luciferases, with blue shifted emissions and increased brightness such as the NanoLuc (NanoBRET, Promega) have increased the sensitivity and popularity of the technology. While potential advantages of BRET include genetic encoding of the energy transfer donor and obviating the need for an excitation light source, BRET ultimately depends on the activity of a luciferase for signal generation and is therefore confined by several fundamental limitations inherent to enzymatic reactions. Besides the need for continuous supply of a substrate, the luciferase activity requires oxygen and is affected by temperature, pH and ionic strength, amongst others. Consequently, BRET is incompatible with both low and high temperatures, unsuitable for anaerobic processes, and sensitive to pH and salt concentration. Total signal intensity is limited by the turnover rate of the luciferase and the emission spectrum limits the number of compatible acceptor fluorophores, which restricts the ability to multiplex. In contrast to TR-FRET, which allows for tight, spatiotemporally controlled excitation of the donor fluorophore, BRET also does not provide meaningful control in these dimensions. Furthermore, the Forster radius of lanthanide TR-FRET pairs is generally larger than that afforded by both conventional FRET and BRET, which enables the probing of larger protein complexes. Lastly. the requirement for the productive spatial orientation of donor and acceptor transition dipoles can critically limit the use of BRET and require lengthy optimization of the linker employed for luciferase fusion protein constructs. In contrast, the Tb-donor emission is not polarized as in BRET⁵⁸, providing for a greater topological flexibility between donor and acceptor.

Despite the advances in TR-FRET probe development presented here, there are still some existing limitations that have not been addressed by our work. Most desirable would be the ability to use TR-FRET probes in vivo. However, the current probes require U V excitation, which has very poor tissue penetration. The development of complexes that can be directly excited by longer wavelength, ideally in the near IR electromagnetic spectrum, would be highly desirable. Unfortunately, this is not possible for Tb-based complexes, for which inherent photophysical properties predict a theoretical upper limit of ˜445 nm. However, 2-photon techniques, upconverting nanoparticles or fiber optics could potentially offer suitable solution

Last, while gel-based TMR-Halo labeling competition experiments provide direct evidence of cellular uptake of Cora-2-Halo, they do not establish the mode(s) by which this complex enters cells and if it preferentially partitions into specific subcellular compartments. The near quantitative labeling of both cytosolic and nuclear target proteins with Cora-2-Halo but not Cora-1-Halo suggests sufficiently broad distribution of the former. Although the requirement for over-night incubation to achieve a high degree of labeling could be the result of intracellular scavenging in subcellular compartments such as lysosomes, we believe that the relatively large size of the core complex is responsible for comparatively slow passive membrane diffusion. This hypothesis of passive uptake is further supported by the fact that Cora-1-Halo exhibits a similar size and overall charge, but less desirable log D. Future time-resolved luminescent imaging studies will be critical to interrogate these processes.

### Preparation of Exemplified Compounds

Reagents & Equipment: reagents and ligands were purchased from Chem-Impex International, Millipore-Sigma, TCI America, Beantown Chemical, Combi-Blocks, Promega, Gaia Chemical Corporation, MedChemExpress, and BOC Sciences. M344-FITC (31) was prepared according to literature procedures. N-LDEETGEFL-CONH₂ (27, N-KL9) and N^{a}-FITC(Ahx)-LDEETGEFL-CONH₂ (17, FITC-KL9) peptides were custom synthesized by Genscript (Piscataway. New Jersey). Recombinant, full length Keap1 constructs either with N-terminal 6×His and GST tags (11981-H20B) or no affinity tags (11981-HNCB) were purchased from Sino Biological Inc. Column purifications were performed on a Biotage Isolera 4 Purification System equipped with a 200-400 nm diode array detector. For normal phase flash purifications, Sorbtech Purity Flash Cartridges were used (CFC-52300-012-18 and CFC-52500-025-12). For reverse phase flash purifications, Biotage Sfar Bio C18 Duo 300 A, 20 μm cartridges were used (FSBD-0411-001). Analytical LC/MS was performed on a Waters 2545 HPLC equipped with a 2998 diode array detector, a 2424 evaporative light scattering detector, a 2475 multichannel fluorescence detector, and a Waters 3100 ESI-MS module, using a XTerraMS C18 5 μm, 4.6×50 mm column at a flow rate of 5 mL/min with a linear gradient (95% A: 5% B to 100% B 90 sec and 30 sec hold at 100% B, solvent A=water+0.1% formic acid, solvent B=acetonitrile+0.1% formic acid). Proton, carbon, and fluorine nuclear magnetic resonance (¹H, ¹³C, and ¹⁹F NMR spectra) were recorded on a Bruker Avance III 400 spectrometer. Chemical shifts for protons are reported in parts per million (ppm) and are referenced to residual solvent peaks. Data is reported as follows: chemical shift, multiplicity (s=singlet, br s, =broad singlet, d=doublet, t=triplet, q=quartet, p=pentet, m=multiplet), proton coupling constants (J, Hz), and integration.

### N-(2-chloroethyl)-4-methylbenzenesulfonamide (37)

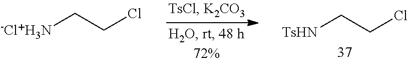

Compound 37 was synthesized according to the literature, with minor modifications. K₂CO₃ (174 g, 1.26 mol, 3 eq) was dissolved in deionized water (1.7 L). To this solution was added 2-chloroethylamine hydrochloride (68.1 g, 587 mmol, 1.4 eq) followed by TsCl (80 g, 420 mmol, 1 eq) and the resulting suspension was stirred vigorously at room temperature for 48 h or until the characteristic aromatic TsCl resonances disappeared by ¹H NMR analysis (CDCl₃). The reaction mixture was filtered and the solid obtained was washed with deionized water and dried in vacuo overnight. Yield=78 g, 72% average yield as a white solid. ¹H NMR (400 MHz, CDCl₃) δ 7.75 (d, J=7.9 Hz, 2H), 7.32 (d, J=7.9 Hz, 2H), 4.95 (s, 1H), 3.55 (t, J=5.8 Hz, 2H), 3.30 (q, J=6.0 Hz, 2H), 2.43 (s, 3H). ¹³C NMR (101 MHz, CDCl₃) δ 143.99, 136.97, 130.02, 127.17, 44.78, 43.76, 21.69.

### 1-tosylaziridine (38)

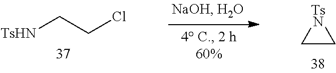

Compound 38 was synthesized according to the literature. N-(2-chloroethyl)-4-methylbenzenesulfonamide (1, 63.7 g, 273 mmol, 1 eq) was suspended in a pre-chilled (4° C.) solution of 1.4 M NaOH (820 mL, 1.14 mol, 4.2 eq) and the resulting suspension was stirred at 4° C. in an ice bath for 2 h or until the disappearance of the characteristic methylene resonances of 1 by ¹H NMR analysis (CDCl₃) was observed. The reaction mixture was allowed to sit at 4° C. overnight to precipitate the product. The solid was filtered, washed with cold deionized water and dried in vacuo overnight. Yield=32.3 g, 60% average yield as a white, crystalline solid. ¹H NMR (400 MHz, CDCl₃) δ 7.84 (d, J=7.9 Hz, 2H), 7.35 (d, J=8.0 Hz, 2H), 2.45 (s, 3H), 2.37 (s, 4H). ¹³C NMR (101 MHz, CDCl₃) δ 144.81, 135.01, 129.89, 128.15, 27.58, 21.80.

### N,N′,N″,N″-((ethane-1,2-diylbis(azanetriyl))tetrakis(ethane-2,1-diyl))tetrakis(4-methylbenzenesulfonamide) (39)

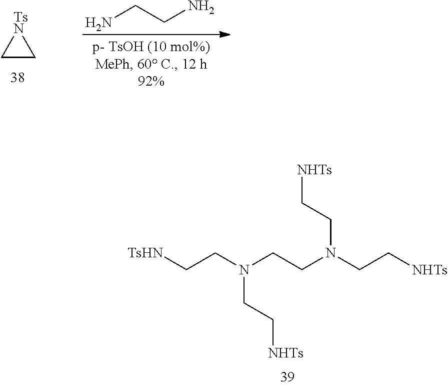

Ethylenediamine (2.6 mL, 2.3 g, 38.3 mmol, 1 eq) was dissolved in MePh (125 mL) then p-TsOH (7.7 mL, 0.5 M in ACN, 3.8 mmol, 0.1 eq) was added followed by 1-tosylaziridine (38, 33.3 g. 168.7 mmol, 4.4 eq) and the resulting reaction mixture was equipped with a reflux condenser and heated to 60° C. for 12 h. The reaction mixture was concentrated to dryness in vacuo and the resulting crude solid was suspended in MeOH (100 mL). To this suspension was added triethylamine (1.1 mL, 0.8 g, 7.6 mmol, 0.2 eq) and the suspension was stirred for 15 min at room temperature. The solid was filtered, washed with MeOH and dried in vacuo overnight. Yield=29.9 g, 92% average yield as a white, crystalline solid. ¹H NMR (400 MHz, DMSO-d₆) δ 7.65 (d, J=7.9 Hz, 8H), 7.36 (s, 4H), 7.34 (d, 8H), 2.63 (t, J=7.1 Hz, 8H), 2.36 (s, 12H), 2.23 (t, J=6.7 Hz, 8H), 2.09 (s, 4H). ¹³C NMR (101 MHz, DMSO-d₆) δ 142.58, 137.69, 129.65, 126.49, 53.19, 51.00, 40.51, 20.98. MS (ESI^{+/-}) m/z (M+H)⁺ 849.36, m/z (M−H)⁻ 847.59, [calculated C₃₈H₅₂N₆O₈S₄: 848.27].

Notes: The addition of p-TsOH was found to significantly reduce overalkylation of the newly formed tosyl amides, while still allowing the reaction to proceed to near completion.

### N1,N1′-(ethane-1,2-diyl)bis(N1-(2-aminoethyl)ethane-1,2-diamine)·6HBr (40)

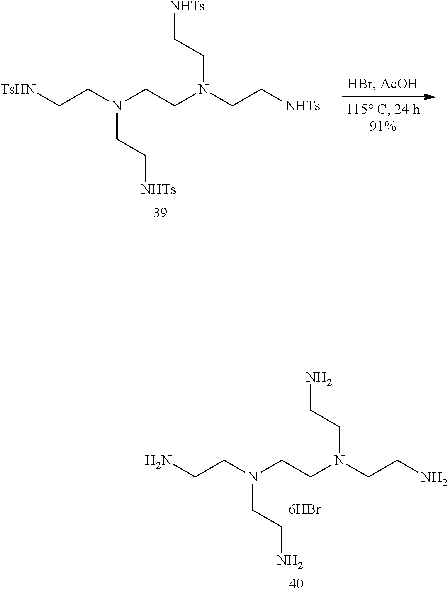

N,N,N′,N′″-((ethane-1,2-diylbis(azanetriyl))tetrakis(ethane-2,1-diyl))tetrakis(4-methylbenzenesulfonamide) (39, 10.0 g, 11.8 mmol) was suspended in 12 mL of 48% wt aqueous HBr and 8 mL of glacial acetic acid and the resulting suspension was heated to 115° C. under reflux conditions for 24 h. The dark red solution was cooled to mom temperature, at which point an off-white solid precipitated. To the reaction mixture was added 20 mL of a 1:1 solution of Et₂O/EtOH to further precipitate the desired product. The solid was filtered, washed with the 1:1 Et₂O/EtOH solution and dried in vacuo overnight. Yield=7.7 g; 91% average yield as a beige, fluffy solid. ¹H NMR (400 MHz, D₂O) δ 3.17 (t, J=6.8 Hz, 8H), 3.01 (t, J=6.8 Hz, 8H), 2.98 (s, 4H). ¹³C NMR (101 MHz, D₂O) 5 50.06, 49.02, 35.77. MS (ESI⁺) m/z (M+H)⁺ 233.35, [calculated C₁₀H₂₈N₆: 232.24].

Notes: If after 24 h of reflux the reaction is not complete via LCMS analysis, the precipitation steps can be performed and the crude solid re-subjected to fresh deprotection conditions without a significant loss in yield.

### 6-((N-(2-((2-(bis(2-((4-methylphenyl)sulfonamido)ethyl)amino)ethyl)(2-((4-methylphenyl)sulfonamido)ethyl)amino)ethyl)-4-methylphenyl)sulfonamido)hexanoic acid (41)

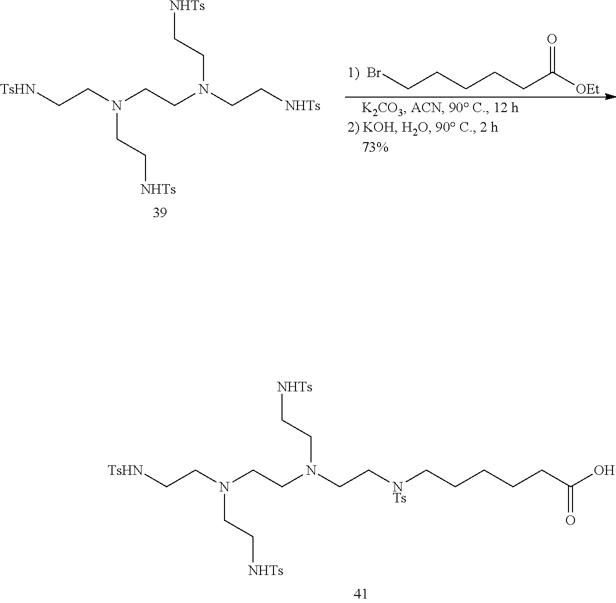

N,N,N′,N″-((ethanc-1,2-diylbis(azanetriyl))tetrakis(ethane-2,1-diyl))tetrakis(4-methylbenzenesulfonamide) (39, 5.0 g, 5.9 mmol, 3.2 eq) and K₂CO₃ (1.1 g, 7.8 mmol, 4.25 eq) were suspended in ACN (125 mL) then ethyl 6-bromohexanoate (327 μL, 0.41 g, 1.8 mmol, 1 eq) was added and the reaction mixture was equipped with a reflux condenser and heated to 90° C. for 12 h. The reaction mixture was filtered while hot to remove carbonate salts. The filtrate was re-heated to 90° C. to dissolve any precipitated starting material then allowed to cool to room temperature before being put at −20° C. overnight to recrystallize unreacted starting material. The solution was filtered and the solid was washed with minimal ACN (2.5 g of 39 recovered). The filtrate was concentrated to dryness in vacuo then the crude product was suspended in 1 N KOH (100 mL) and heated to 90° C. for 2 h, or until all solid went into solution. The pH was adjusted to 2-3 with 12 N HCl to precipitate the crude, saponified product as a white, waxy solid. The mother liquor was decanted and the solid was washed with water. The crude solid was suspended in hot MePh, concentrated and dried in vacuo to remove residual water. The crude product was purified via flash chromatography (dry load; λ230 nm, 263 nm; gradient: 100% DCM for 2 CV, 100% DCM to 4% MeOH/DCM over 4 CV, 4% MeOH/DCM for 4 CV, 4% to 10% MeOH/DCM over 2 CV, 10% MeOH/DCM for 3 CV). Additional starting material (39) is also recovered from the column. Yield=1.3 g, 73% average yield as a white solid. Total starting material recovered=3.1 g. ¹H NMR (400 MHz, CDCl₃) δ 7.75 (d, J=7.9 Hz, 2H), 7.71 (d, J=7.9 Hz, 4H), 7.66 (d, J=7.9 Hz, 2H), 7.30 (d, 2H), 7.27 (d, J=8.2 Hz, 6H), 6.23 (s, 3H), 3.13 (t, J=7.3 Hz, 2H), 3.02 (t, J=7.4 Hz, 2H), 2.97 (t, J=5.6 Hz, 2H), 2.89 (t, J=5.3 Hz, 4H), 2.69 (t, J=7.4 Hz, 2H), 2.63 (q, J=7.2, 6.7 Hz, 4H), 2.52-2.44 (m, 6H), 2.41 (s, 3H), 2.39 (s, 6H), 2.38 (s, 3H), 2.35 (t, J=7.0 Hz, 2H), 1.62 (p, J=7.0 Hz, 2H), 1.48 (p, J=7.3 Hz, 2H), 1.30 (p, J=7.7 Hz, 2H). ¹³C NMR (101 MHz, CDCl₃) δ 178.77, 143.59, 143.49, 136.97, 136.83, 136.81, 136.17, 136.15, 129.94, 129.88, 127.33, 127.28, 127.18, 54.08, 53.69, 53.50, 52.48, 51.56, 49.32, 45.88, 40.61, 34.20, 28.02, 26.03, 24.20, 21.65. MS (ESI^{+/-}) m/z (M+H)⁺ 963.34, m/z (M−H)⁻ 961.61. [calculated C₄₄H₆₂N₆O₁₀S₄: 962.34].

Notes: Starting material recovered from recrystallization and column steps can be combined and re-subjected to alkylation conditions without any noticeable loss in yield. The crude ethyl ester product is saponified to aid in separation during column purification.

### Ethyl 6-((2-((2-aminoethyl)(2-(bis(2-aminoethyl)amino)ethyl)amino)ethyl)amino)hexanoate-6HBr (42)

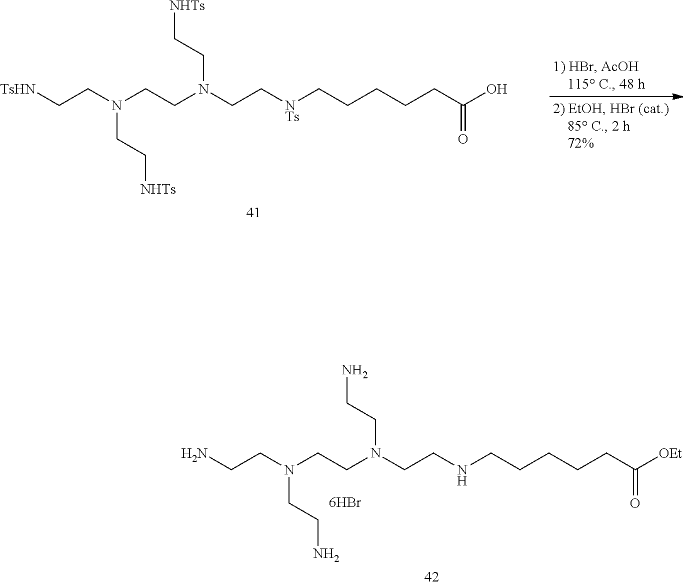

6-((N-(2-((2-(bis(2-((4-methylphenyl)sulfonamido)ethyl)amino)ethyl)(2-((4-methylphenyl)sulfonamido)ethyl)amino)ethyl)-4-methylphenyl)sulfonamido)hexanoic acid (41, 1.03 g, 1.07 mmol) was suspended in 3 mL 48% wt HBr and 2 mL of glacial acetic acid and the resulting suspension was heated to 115° C. under reflux conditions for 48 h. The solution was cooled to room temperature and 15 mL 1:1 EtOH/Et₂O solution was added to precipitate the crude product as a waxy solid. The precipitate was allowed to collect in an ice bath for 10 min. The mother liquor was decanted and the solid was washed twice more with the EtOH/Et₂O solution and dried in vacuo. Once dried, the crude solid was re-dissolved in 2 mL 48% wt HBr and the precipitation process was repeated to ensure complete removal of acetate/p-TsOH salts. The resulting dried solid was then suspended in EtOH (10 mL) and 48% wt HBr (10 μL, 107 μmol, 0.1 eq) was added and the suspension was heated to 85° C. under reflux conditions for 2 h or until all solid went into solution. The reaction mixture was cooled to room temperature then further cooled in an ice bath to precipitate the product, which was collected via centrifugation, washed with cold ethanol and dried overnight in vacuo. Yield=660 mg, 72% average yield as an off-white solid. ¹H NMR (400 MHz, D₂O) δ 4.15 (q, J=7.0 Hz, 2H), 3.40-3.17 (m, 20H), 3.13 (t, J=7.2 Hz, 2H), 2.40 (t, J=7.1 Hz, 2H), 1.74 (p, J=7.0 Hz, 2H), 1.63 (p, J=7.2 Hz, 2H), 1.41 (p, J=7.1 Hz, 2H), 1.24 (t, J=7.0 Hz, 3H). ¹³C NMR (101 MHz, D₂O) S 176.92, 61.74, 50.04, 49.35, 49.07, 48.98, 48.09, 43.37, 35.64, 35.38, 33.71, 25.26, 25.20, 23.75, 13.41. MS (ESI⁺) m/z (M+H)⁺ 375.51. [calculated C₁₈H₄₂N₅O₂: 374.34].

Notes: It was found that repeating the precipitation process after initial deprotection from fresh HBr significantly helped to remove unwanted salts (acetate/p-TsOH) trapped in the waxy solid. The crude solid exists as an extremely waxy material, but upon conversion to the ethyl ester will form a free-flowing powder. During the esterification step, the solid will not fully dissolve in the ethanol until a true reflux is achieved.

### 2-methoxyisophthalic Acid (X═H; 43)

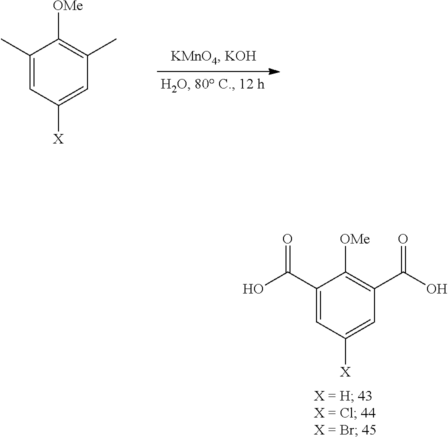

General Procedure A: Into 450 mL of deionized water was dissolved KOH (13.8 g, 247 mmol, 3.2 eq) then KMnO₄ (80.4 g, 509 mmol, 6.6 eq) was added, followed by 2,6-dimethylanisole (10.9 mL, 10.6 g, 77.1 mmol, 1 eq). The reaction mixture was equipped with a reflux condenser and vigorously stirred at 80° C. for 12 h. The reaction mixture was cooled to room temperature and filtered to remove MnO₂. The filtrate was acidified to pH 1 with 12 N HCl to precipitate the product as white crystals. The filtrate was cooled to 4° C. overnight to aid in precipitation and the resulting solid was filtered. The solid was washed with deionized water and dried in vacuo overnight. Yield=12.8 g, 84% average yield as a white, crystalline solid. ¹H NMR (400 MHz, DMSO-d₆) δ 13.12 (s, 1H), 7.81 (d, J=7.7 Hz, 2H), 7.25 (t, J=7.7 Hz, 1H), 3.80 (s, 3H). ¹³C NMR (101 MHz, DMSO-d₆) δ 167.08, 157.78, 133.57, 127.80, 123.66, 63.03. MS (ESI⁻) m/z (M−H)⁻ 195.12, [calculated C₉H₈O₅: 196.04].

### 5-chloro-2-methoxyisophthalic acid (X═Cl; 44)

Following General Procedure A, substituting dimethylanisole with 4-chloro-2,6-dimethylanisole (64). The reaction was run on a 4.9 g (29 mmol) scale of 64. Yield=3.2 g, 48% average yield as a slightly yellow, crystalline solid. ¹H NMR (400 MHz, DMSO-d₆) δ 13.47 (s, 2H), 7.82 (s, 2H), 3.80 (s, 3H). ¹³C NMR (101 MHz, DMSO-d₆) δ 165.77, 156.39, 132.66, 129.76, 127.42, 63.28, 39.52. MS (ESI⁻) m/z (M−H)⁻ 229.10, [calculated C₉H₇ClO₅: 230.00].

### 5-bromo-2-methoxyisophthalic acid (X═Br; 45)

Following General Procedure A, substituting dimethylanisole with 4-bromo-2,6-dimethylanisole. The reaction was run on a 16.0 g (74 mmol) scale of 4-bromo-2,6-dimethylanisole. Yield=8.5 g, 41% average yield as an off-white, fluffy solid. ¹H NMR (400 MHz, DMSO-d₆) δ 13.48 (s, 2H), 7.94 (s, 2H), 3.80 (s, 3H). ¹³C NMR (101 MHz, DMSO-d₆) δ 165.65, 156.82, 135.51, 130.03, 115.09, 63.21. MS (ESI) m/z (M−H)⁻ 273.15, [calculated C₉H₇BrO₅: 273.95].

### 2-methoxy-3-((2,3,5,6-tetrafluorophenoxy)carbonyl)benzoic acid (X═H; 46)

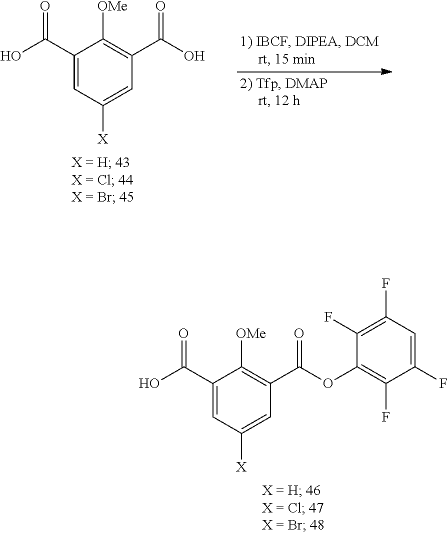

General Procedure B: 2-methoxyisophthalic acid (43, 3.0 g, 15.3 mmol, 1 eq) and DIPEA (10.7 mL, 7.9 g, 61.2 mmol, 4 eq) were dissolved in DCM (100 mL). Isobutyl chloroformate (IBCF, 2.2 mL, 2.3 g, 16.8 mmol, 1.1 eq) was added dropwise as a solution in 10 mL DCM over 15 min via a syringe pump. The reaction mixture was stirred for 15 min at room temperature then 2,3,5,6-tetrafluorophenol (3.1 g, 18.4 mmol, 1.2 eq) was added, followed by DMAP (93 mg, 0.8 mmol, 0.05 eq) upon complete dissolution of the 2,3,5,6-tetrafluorophenol. Upon addition of DMAP, vigorous gas evolution occurred. The reaction was stirred at room temperature for 12 h to allow for equilibration. The mixture was diluted into 100 mL DCM and the organic layer was washed 2×200 mL 0.2 N HCl, keeping the aqueous washes at pH ˜1. The product was extracted with 3×200 mL 0.2 N NaHCO₃, with minimal saturated brine solution being added to separate layers if necessary. The combined aqueous layers were slowly acidified to pH 1 with 12 N HCl to precipitate a white, fluffy solid, which was collected via filtration, washed with water then dried in vacuo overnight. Yield=3.8 g, 80% average yield as a white solid. ¹H NMR (400 MHz, CDCl₃) δ 8.33 (d, J=2.7 Hz, 1H), 8.26 (d, J=2.9 Hz, 1H), 7.10 (p, J=8.6, 7.3 Hz, 1H), 4.08 (s, 3H). ¹³C NMR (101 MHz, CDCl₃) δ 165.53, 159.52, 159.47, 147.48, 145.05, 142.10, 139.56, 138.09, 137.09, 136.60, 130.53, 126.19, 124.16, 104.10, 77.16, 65.09. ⁹F NMR (377 MHz, CDCl₃) δ −138.18, −152.53. MS (ESI⁻) m/z (M−H)⁻ 343.23, [calculated C₁₅H₈F₄O₅: 344.03].

Notes: Over the course of 12 h in the presence of DMAP, the complicated mixture of products eventually equilibrates into predominantly (>85%) the desired mono-Tfp ester. When extracting the product into aqueous NaHCO₃ solution, the organic and aqueous layers may take extended periods of time to separate (˜10-15 min) and a small amount of brine can be added to aid in separation. We found that the Tfp ester (as opposed to the Pfp ester) provided superior stability to hydrolysis during these workup steps, while maintaining adequate reactivity in subsequent coupling reactions. The product may also be purified via column chromatography if desired (post-acidic workup), using Hex/EtOAc as the eluent. However, on larger scales, we found the extraction-precipitation method to be more reliable.

### 5-chloro-2-methoxy-3-((2,3,5,6-tetrafluorophenoxy)carbonyl)benzoic acid (X═Cl; 47)

Following General Procedure B, substituting 43 with 44. The reaction was run on a 1.0 g (4.3 mmol) scale of 44. Yield=1.2 g, 70% average yield as a white solid. ¹H NMR (400 MHz, CDCl₃) δ 8.33 (d, J=2.7 Hz, 1H), 8.26 (d, J=2.9 Hz, 1H), 7.10 (p, J=8.6, 7.3 Hz, 1H), 4.08 (s, 3H). ¹³C NMR (101 MHz, CDCl₃) δ 165.53, 159.52, 159.47, 147.48, 145.05, 142.10, 139.56, 138.09, 137.09, 136.60, 130.53, 126.19, 124.16, 104.10, 77.16, 65.09. ¹⁹F NMR (377 MHz, CDCl₃) δ −138.18, −152.53. MS (ESI⁻) m/z (M−H)⁻ 377.20, [calculated C₁₅H₇ClF₄O₅: 377.99].

### 5-bromo-2-methoxy-3-((2,3,5,6-tetrafluorophenoxy)carbonyl)benzoic acid (X═Br; 48)

Following General Procedure B. substituting 43 with 45. The reaction was run on a 2.0 g (7.3 mmol) scale of 45. Yield=2.3 g, 75% average yield as a white solid. ¹H NMR (400 MHz, CDCl₃) δ 8.46 (s, 1H), 8.40 (s, 1H), 7.10 (p, J=9.3, 8.8 Hz, 1H), 4.08 (s, 3H). ¹³C NMR (101 MHz, CDCl₃) 166.00, 160.18, 159.43, 147.49, 145.01, 141.96, 140.98, 139.95, 139.47, 129.31, 126.52, 124.59, 117.47, 104.08, 77.16, 64.98. ¹⁹F NMR (377 MHz, CDCl₃) δ −138.21, -152.50. MS (ESI⁻) m/z (M−H) 421.20, [calculated C₁₅H₇BrF₄O₅: 421.94].

### N1,N1,N2-tris(2-(3-carboxy-2-methoxybenzamido)ethyl)-N2-(2-(3-carboxy-N-(6-ethoxy-6-oxohexyl)-2-methoxybenzamido)ethyl)ethane-1,2-diamine·2HCl (X═H; 49)

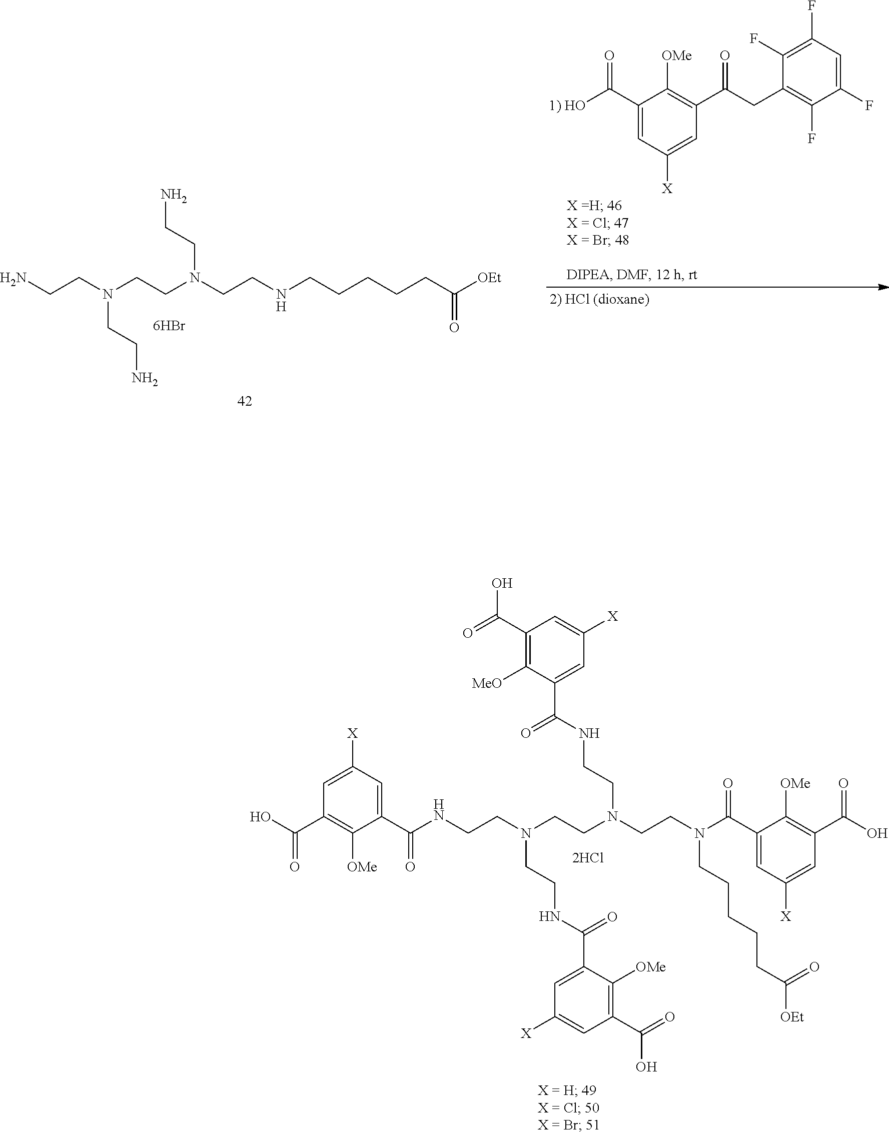

General Procedure C: Ethyl 6-((2-((2-aminoethyl)(2-(bis(2-aminoethyl)amino)ethyl)amino)ethyl)amino)hexanoate-6HBr (42, 150 mg, 174 μmol, 1.0 eq) was dissolved in DMF (2 mL) with DIPEA (425 μL, 316 mg, 2.4 mmol, 14 eq) then 2-methoxy-3-((2,3,5,6-tetrafluorophenoxy)carbonyl)benzoic acid (46, 243 mg, 706 μmol, 4.05 eq) was added as a solution in 1 mL DMF. The reaction mixture was stirred for 12 h at room temperature then cooled to 4° C. in an ice bath. Once cooled, HCl/dioxane (4.0 M. 872 μL, 3.5 mmol, 20 eq) was added. The reaction was allowed to come to room temperature and was stirred for 15 min to ensure complete acidification of all basic species. The reaction mixture was concentrated in vacuo to an oil, which was first triturated with a mixture of 9:1 Et₂O/ACN (20 mL) to precipitate the crude product as a waxy solid/oil. The solid was allowed to precipitate for 5 min then the mother liquor was carefully decanted and the solid was dried in vacuo for 30 min or until it formed a white foam. The crude product was then triturated 3×5 mL ACN, purified via centrifugation, and dried overnight in vacuo. Yield=199 mg, >95% as a white, fluffy solid. ¹H NMR (400 MHz, D₂O/NaOD) δ 7.48-7.32 (m, 7H), 7.20-7.02 (m, 5H), 3.74-3.56 (m, 13H), 3.49-3.41 (m, 3H), 3.35 (t, J=6.9 Hz, 2H), 3.24-3.16 (m, 3H), 3.07 (t, J=7.5 Hz, 1H), 2.81-2.73 (m, 5H), 2.70-2.58 (m, 3H), 2.44-2.40 (m, 4H), 2.08 (t, J=7.5 Hz, 1H), 1.91 (t, J=7.5 Hz, 1H), 1.61-1.13 (m, 5H), 0.98-0.91 (m, 11H). ¹¹C NMR (101 MHz, D₂O/NaOD) δ 183.71, 183.50, 175.43, 175.39, 174.76, 171.19, 169.25, 169.17, 153.68, 151.92, 143.04, 133.67, 132.46, 130.98, 129.72, 129.46, 129.18, 128.08, 127.65, 127.01, 124.07, 109.29, 62.33, 62.03, 52.30, 52.11, 44.17, 37.47, 37.39, 37.25, 27.65, 26.53, 26.02, 25.63, 25.18. MS (ESI^{+/-}) m/z (M+H)⁺ 1087.67, m/z (M−H)⁻ 1085.62, [calculated C₅₄H₆₆N₆O₁₈: 1086.44].

Notes: During the first precipitation step from Et₂O/ACN, the crude product sometimes will form a separate oily layer instead of being a true solid, in which case the mother liquor can be carefully decanted while retaining the oil. Once high vacuum is applied to the oil, it will form a white foam, at which point the further washes with ACN can be performed. Sonication is recommended during the ACN washes.

### N1,N1,N2-tris(2-(3-carboxy-5-chloro-2-methoxybenzamido)ethyl)-N2-(2-(3-carboxy-5-chloro-N-(6-ethoxy-6-oxohexyl)-2-methoxybenzamido)ethyl)ethane-1,2-diamine-2HCl (X═Cl; 50)

Following General Procedure C, substituting 46 with 47. The reaction was run on an 80 mg (93 μmol) scale of 42. Yield=120 mg, >95% as a white, fluffy powder. ¹H NMR (400 MHz, D₂O/NaOD) δ 7.51-7.13 (m, 8H), 3.78-3.59 (m, 13H), 3.56-3.43 (m, 4H), 3.39 (t, J=7.0 Hz, 2H), 3.32-3.18 (m, 2H), 3.12 (t, J=7.9 Hz, 1H), 2.85-2.75 (m, 5H), 2.73-2.61 (m, 3H), 2.60-2.36 (m, 4H), 2.12 (t, J=7.5 Hz, 1H), 1.99 (t, J=7.5 Hz, 1H), 1.68-1.36 (m, 3H), 1.35-1.21 (m, 2H), 1.15-0.91 (m, 1H). ¹³C NMR (101 MHz, D₂O) δ 183.72, 183.49, 173.65, 173.56, 173.01, 169.55, 167.71, 167.60, 167.56, 152.60, 152.50, 150.80, 150.59, 135.37, 135.29, 134.93, 134.54, 131.12, 130.80, 130.66, 130.50, 129.73, 129.28, 129.20, 128.98. 128.55, 128.42, 127.15, 62.50, 62.21, 62.02, 52.26, 52.11, 51.95, 50.98, 49.65, 46.09, 45.81, 37.49, 37.41, 37.31, 37.00, 27.40, 26.49, 26.04, 25.64, 25.54, 25.22. MS (ESI^{+/-}) m/z (M+H)⁺ 1223.58, m/z (M−H)⁻ 1221.38, [calculated C₅₄H₆₂Cl₄N₆O₁₈: 1222.29].

### N1,N1,N2-tris(2-(3-carboxy-5-bromo-2-methoxybenzamido)ethyl)-N2-(2-(3-carboxy-5-bromo-N-(6-ethoxy-6-oxohexyl)-2-methoxybenzamido)ethyl)ethane-1,2-diamine-2HCl (X═Br; 51)

Following General Procedure C, substituting 46 with 48. The reaction was run on a 90 mg (105 μmol) scale of 42. Yield=150 mg, >95% as a white, fluffy powder. ¹H NMR (400 MHz, D₂O/NaOD) 6 7.69-7.37 (m, 8H), 3.83-3.74 (m, 12H), 3.74-3.66 (m, 1H), 3.58-3.49 (m, 3H), 3.45 (t, J=6.8 Hz, 2H), 3.35-3.26 (m, 3H), 3.17 (t, J=7.4 Hz, 1H), 2.91-2.80 (m, 51H), 2.71 (t, J=6.8 Hz, 3H), 2.62-2.47 (m, 4H), 2.18 (t, J=7.5 Hz, 1H), 2.04 (t, J=7.6 Hz, 1H), 1.70-1.42 (m, 3H), 1.35 (p, J=8.0 Hz, 2H), 1.17-1.01 (m, 1H). ¹³C NMR (101 MHz, D₂O/NaOD) δ 183.75, 183.52, 173.56, 173.47, 172.93, 169.48, 167.66, 167.56, 167.51, 153.13, 153.02, 151.32, 151.15, 135.64, 135.55, 135.22, 134.82, 133.64, 133.45, 132.67, 131.93, 131.45, 131.13, 130.03, 129.61, 129.54, 115.95, 115.83, 62.44, 62.13, 61.94, 57.37, 52.27, 52.13, 51.91, 51.05, 50.77, 49.70, 45.87, 42.39, 37.52, 37.47, 37.39, 37.01, 27.45, 26.52, 26.08, 25.67, 25.59, 25.27. MS (ESI^{+/-}) m/z (M+H) 1399.56, m/z (M−H)⁻ 1397.49, [calculated C₃₄H₆₂Br₄N₆O₁₈: 1398.09].

### Me₄BH(2,2)IAM-N-ethyl hexanoate (X═H; 52)

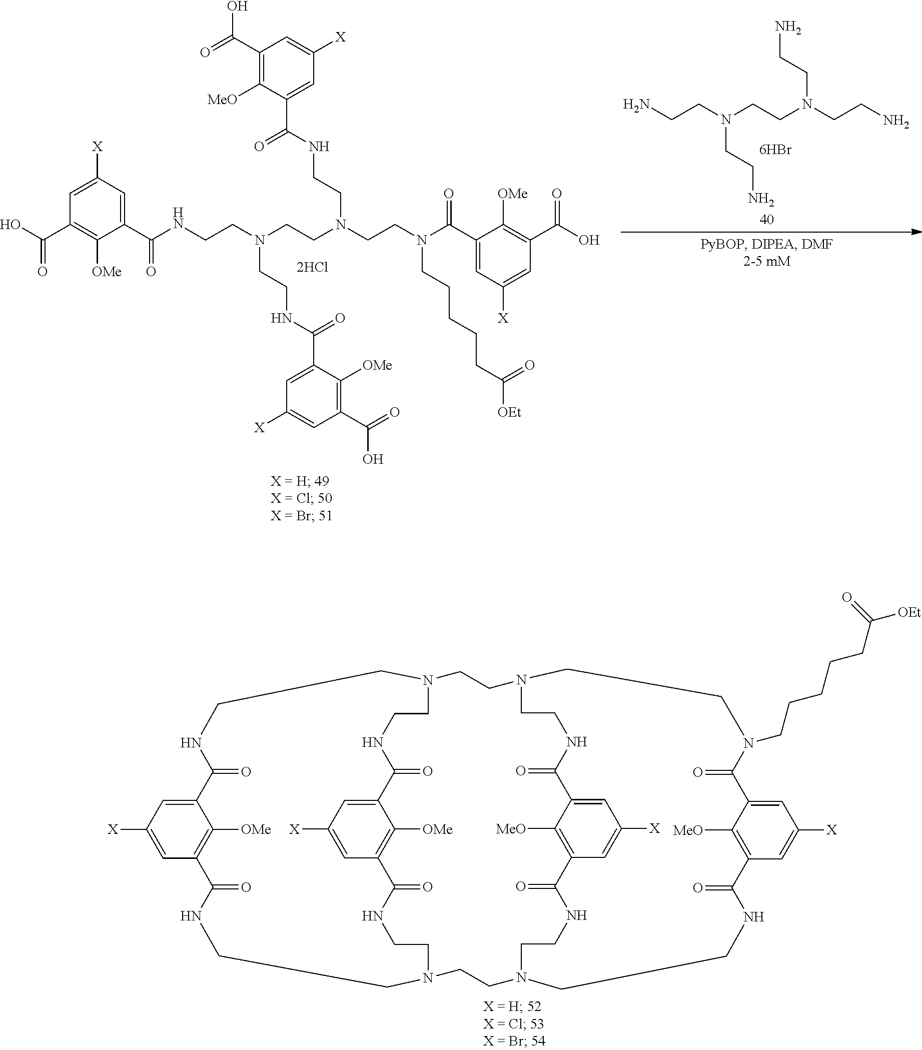

General Procedure D): N1,N1,N2-tris(2-(3-carboxy-2-methoxybenzamido)ethyl)-N2-(2-(3-carboxy-N-(6-ethoxy-6-oxohexyl)-2-methoxybenzamido)ethyl)ethane-1,2-diamine-2HCl (49, 135 mg, 116 μmol, 1.05 eq) was dissolved in DMF (50 mL, ˜2 mM) with DIPEA (580 μL, 430 mg, 3.3 mmol, 30 eq) then N1,N1′-(ethane-1,2-diyl)bis(NI-(2-aminoethyl)ethane-1,2-diamine)-6HBr (40, 80 mg, 111 μmol, 1 eq) was added to create a slightly cloudy solution. In a separate container, PyBOP (346 mg, 665 μmol, 6 eq) was dissolved in DMF (11 mL, ˜60 mM). The solution of PyBOP/DMF was added dropwise to the solution of 40 and 49 in DMF via a syringe pump (5 mL/h or I drop every 3-4 see) over approximately 2 h with vigorous stirring. Upon complete addition of the PyBOP, the reaction mixture became a clear solution. The reaction mixture was allowed to stir at room temperature for 30 min then was concentrated in vacuo to an oil (water bath temp 40-50° C.). The crude product was precipitated first as a waxy solid with 9:1 Et₂O/ACN (20 mL). The product was allowed to precipitate for 5 min then the mother liquor was carefully decanted and the trituration process repeated twice more. The crude solid was dried in vacuo for 30 min or until it became a white foam. The solid was purified via flash chromatography (λ 225 nm, 285 nm; 6% MeOH/DCM+1% NH₄OH). Yield=89 mg, 68% average yield as a white, crystalline solid. ¹H NMR (400 MHz, MeOD) δ 8.00-6.94 (m, 12H), 4.08 (q, J=7.4, 6.9 Hz, 2H), 3.91-3.40 (m, 28H), 3.23-2.46 (m, 24H), 2.30 (t, J=7.3 Hz, 21-1), 1.96-1.52 (m, 4H), 1.44-1.36 (m, 2H), 1.30-1.18 (m, 5H). ¹³C NMR (101 MHz, MeOD) δ 175.32, 175.14, 170.81, 133.72, 125.53, 63.86, 61.40, 38.85, 34.91, 34.66, 30.76, 28.26, 27.32, 26.82, 25.71, 14.56. MS (ESI/) m/z (M+H)⁺ 1247.90, m/z (M−H) 1245.76, [calculated C₆₄H₈₆N₁₂O₁₄: 1246.64].

Notes: If solid begins to accumulate on the sides of the reaction flask during dropwise addition of PyBOP. the addition may be paused and the flask sonicated to resuspend/homogenize the cloudy mixture. This process is usually required once or twice during a 2 h addition. After complete addition of PyBOP, however, the mixture should be completely clear (i.e. all solid dissolved). When wet loading the column in MeOH/DCM/NH₄OH solution, not all solid will dissolve. The undissolved solid will consist mostly of unwanted polymeric material. As such, filtering the wet load solution through a 0.22 μm PVDF syringe filter to remove the polymeric byproduct is the method of choice. Dry loading is also acceptable as the polymer remains baseline during column purification. The most common co-eluting impurity from the column is residual DIPEA, which does not have to be removed before the final deprotection step.

Me₄Cl₄BH(2,2)IAM-N-ethyl hexanoate (X═Cl; 53)

Following General Procedure D, substituting 49 with 50. The reaction was run on a 111 mg (154 μmol) scale of 40. Flash chromatography conditions: a 225 nm, 295 nm, 5% MeOH/DCM+1% NH₄OH. Yield=149 mg, 70% as a white, crystalline solid. ¹H NMR (400 MHz, CDCl₃) δ 8.32-6.91 (m, 15H), 4.09 (tt, J=14.2, 7.0 Hz, 2H), 3.94-3.71 (m, 8H), 3.71-3.28 (m, 18H), 3.19-2.53 (m, 24H), 2.42-2.13 (m, 4H), 1.98-1.76 (m, 2H), 1.63 (dt, J=15.2, 7.9 Hz, 2H), 1.53-1.33 (m, 3H), 1.23 (dt, J=14.2, 6.6 Hz, 4H). ¹³C NMR (101 MHz, CDCl₃) δ 173.66, 166.08, 165.49, 164.84, 164.68, 164.29, 163.89, 163.53, 154.06, 152.45, 133.89, 133.43, 133.20. 132.93, 131.75, 130.81, 130.64, 130.24, 128.74, 128.55, 63.90, 63.53, 63.23, 62.75, 60.44, 60.39, 53.38, 46.39, 41.83, 37.71, 34.18, 33.90, 32.03, 29.81, 27.75, 26.63, 26.44, 26.01, 24.71, 24.64, 24.31, 22.80, 18.36, 14.35, 12.70. MS (ESI^{+/-}) m/z (M+H)⁺ 1383.64, m/z (M−H)⁻ 1381.54, [calculated C₆₄H₈₂C₁₄N₁₂O₁₄: 1382.48].

### Me₄Br₄BH(2,2)IAM-N-ethyl hexanoate (X═Br; 54)

Following General Procedure D, substituting 49 with 51. The reaction was run on a 71 mg (99 μmol) scale of 40. Flash chromatography conditions: 1225 nm, 295 nm; 4% MeOH/DCM+1% NH₄OH. Yield=108 mg, 69% as a white, crystalline solid. ¹H NMR (400 MHz, CDCl₃) δ 8.48-7.29 (m, 13H), 4.23-4.00 (m, 3H), 3.95-3.24 (m, 25H), 3.24-2.39 (m, 24H), 2.39-2.03 (m, 4H), 1.73-1.56 (m, 2H), 1.54-1.36 (m, 4H), 1.28-1.16 (m, 5H), 1.14-0.72 (m, 2H). ¹³C NMR (101 MHz, CDCl₃) δ 173.74, 173.49, 163.95, 154.71, 136.47, 117.88, 63.86, 60.49, 53.96, 42.17, 37.83, 34.25, 32.05, 29.83, 27.72, 26.06, 24.31, 22.82, 18.72, 17.48, 14.37, 12.18. MS (ESI⁺) m/z (M+H)⁺ 1559.86, m/z (M−H)⁻ 1557.65, [calculated C₆₄H₈₂Br₄N₁₂O₁₄: 1558.28].

### BH(2,2)IAM-N-hexanoic acid·4HBr (X═H; CoraFluor-1 ligand; 1)

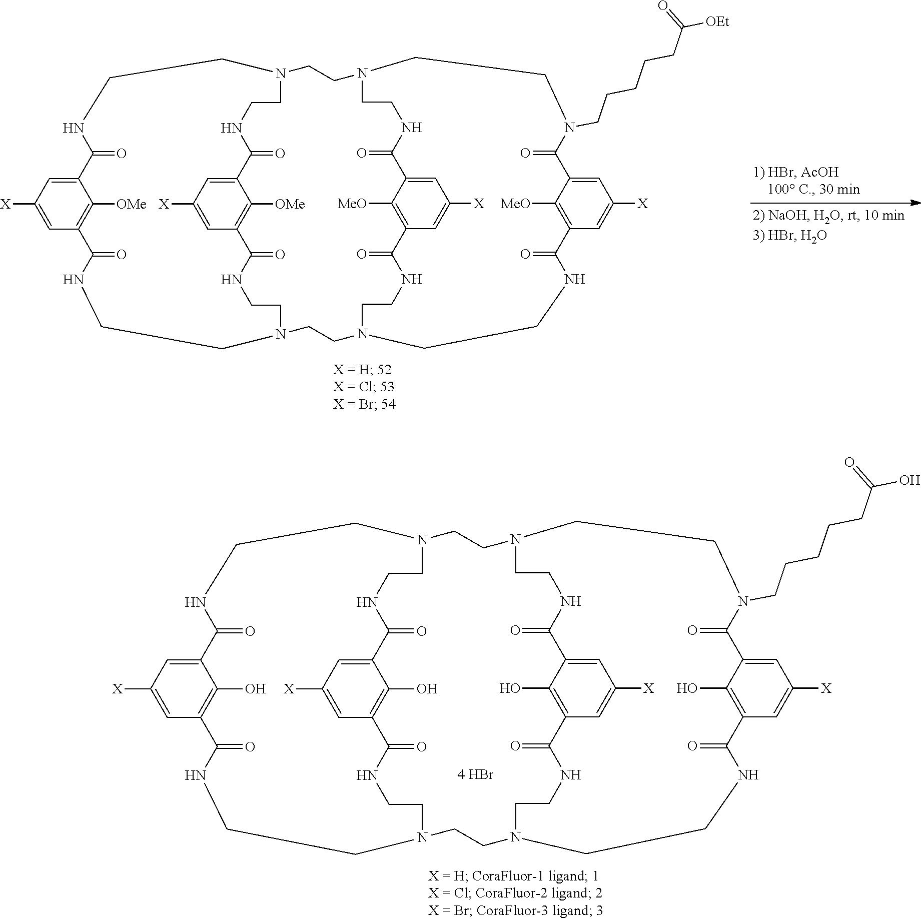

General Procedure E: Me₄BH(2,2)IAM-N-ethyl hexanoate (52, 34 mg, 27 μmol, 1 eq) was dissolved in AcOH (2 mL, −10 mM) then 48% wt aqueous HBr (62 μL, 550 μmol, 20 eq) was added and the reaction mixture was heated to 100° C. under reflux conditions for 30 min, at which point LCMS analysis indicated quantitative methyl ether deprotection and ˜75% ethyl ester hydrolysis. To the room temperature reaction mixture was added 1.5 mL Et₂O to precipitate the crude product, which was collected via centrifugation, washed 2×1.5 mL Et₂O, and dried in vacuo for 30 min. The crude product was dissolved in 300 μL 1 N NaOH and the solution was allowed to stand for 10 min at room temperature to saponify the remaining ethyl ester-protected ligand. The reaction mixture was acidified to pH 1 with 100 μL 48% wt aqueous HBr to precipitate the product as a white solid, which was collected via centrifugation, washed 2×200 μL H₂O, and dried in vacuo for 2-3 h or until residual H₂O was removed. Once dry, the product was triturated 2×500 μL ACN (with sonication), purified via centrifugation, and dried in vacuo overnight. Yield=39 mg, >95% as a white powder. ¹H NMR (400 MHz, D₂O/NaOD) δ 7.98-7.02 (m, 8H), 6.60-6.28 (m, 4H), 3.70-3.59 (m, 2H), 3.56-3.26 (m, 8H), 3.24-3.03 (m, 6H), 3.01-2.51 (m, 22H), 2.47-2.25 (m, 3H), 2.18-1.82 (m, 3H), 1.62-1.43 (m, 4H), 1.28-1.07 (m, 2H). MS (ESI^{+/-}) m/z (M+H)⁺ 1163.75, m/z (M−H) 1161.70, [calculated C₅₈H₇₄N₁₂O₁₄: 1162.54].

Notes: After washing the completely saponified product with H₂O, the material must be thoroughly dried in order to avoid a mixture of H₂O/ACN during ACN washes.

### Cl₄BH(2,2)IAM-N-hexanoic acid-4HBr (X═Cl; CoraFluor-2 ligand; 2)

Following General Procedure E, substituting 52 with 53. The reaction was run on a 16 mg (11 μmol) scale of 53. Yield=18 mg, >95% as a white powder. ¹H NMR (400 MHz, D₂O/NaOD) δ 7.86-6.97 (m, 8H), 3.76-2.93 (m, 20H), 2.86-2.48 (m, 20H), 2.40-2.30 (m, 2H), 2.26-2.15 (m, 1H), 2.10-1.98 (m, 2H), 1.55-1.06 (m, 5H). MS (ESI^{+/-}) m/z (M+H)⁺ 1299.69, m/z (M−H)⁻ 1297.38, [calculated C₅₈H₇₀Cl₄N₁₂O₁₄: 1298.39].

### Br₄BH(2,2)IAM-N-hexanoic acid-4HBr (X═Br; CoraFluor-3 ligand; 3)

Following General Procedure E, substituting 52 with 54. The reaction was run on a 30 mg (19 μmol) scale of 54. Yield=33 mg, >95% as a white powder. ¹H NMR (400 MHz, D₂O/NaOD) δ 8.11-7.11 (m, 8H), 3.84-3.00 (m, 19H), 3.00-2.50 (m, 2H), 2.48-2.21 (m, 3H), 2.20-2.03 (m, 2H), 1.65-1.36 (m, 3H), 1.32-0.83 (m, 2H). MS (ESI^{+/-}) m/z (M+H)⁺ 1475.81, m/z (M−H)⁻ 1473.61, [calculated C₅₈H₇₀Br₄N₁₂O₄: 1474.19].

### BH(2,2)IAM-N-HaloTag·4HBr (X═H; CoraFluor-1-HaloTag ligand; 55)

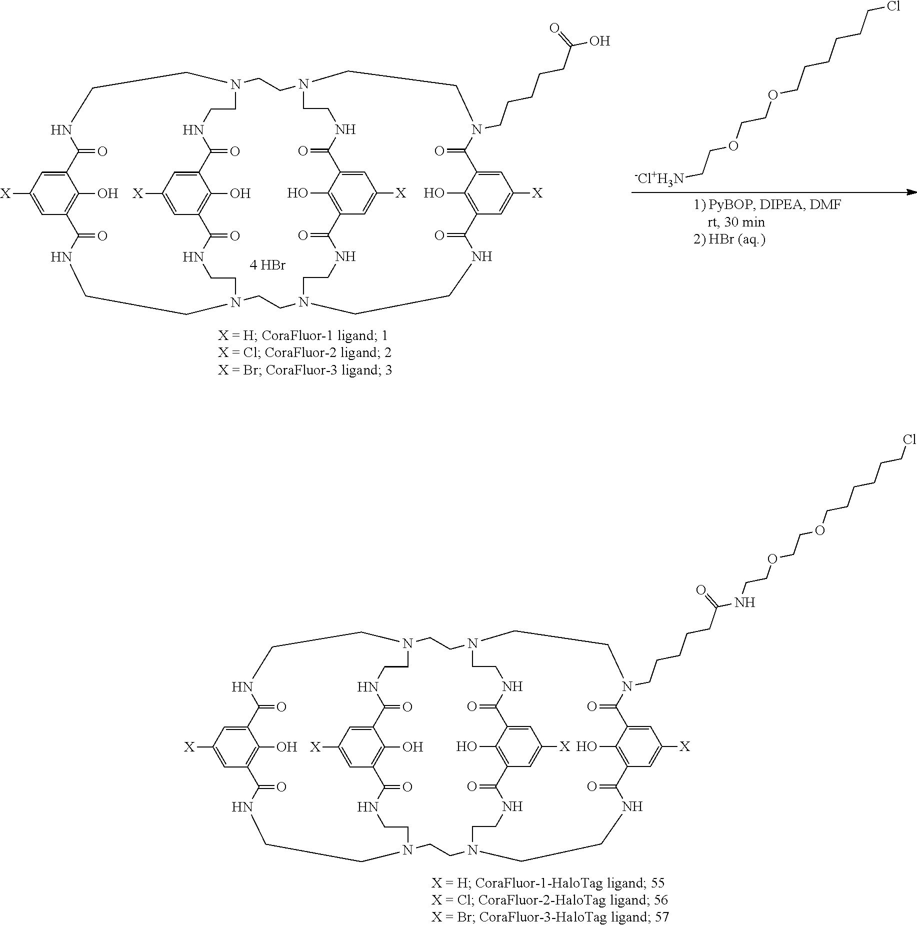

General Procedure F: BH(2,2)IAM-N-hexanoic acid·4HBr (X═H; CoraFluor-1 ligand; 1, 11.5 mg, 8 μmol, 1 eq) was dissolved in DMF (500 μL, ˜10 mM) with DIPEA (27 μL, 20 mg, 155 μmol, 20 eq) then 2-(2-((6-chlorohexyl)oxy)ethoxy)ethan-1-aminium chloride (2.5 mg, 100 mM in DMF, 97 μL, 10 μmol, 1.25 eq) was added followed by PyBOP (6 mg, 100 mM in DMF, 116 μL, 12 μmol, 1.5 eq) and the reaction mixture was stirred at room temperature for 30 min. To the reaction mixture was added 48% wt aqueous HBr (22 μL, 193 μmol, 25 eq) and the reaction mixture was concentrated to an oil in vacuo (water bath 40° C.). The product was precipitated via the addition of 2 mL of 1:1 Et₂O/ACN solution (with sonication), purified via centrifugation, washed 2×500 μL ACN, and dried overnight in vacuo. Yield=12.5 mg, >95% as a white powder. MS (ESI^{+/-}) m/z (M+H)⁺ 1368.84, m/z (M−H)⁻ 1366.68. [calculated C₆₈H₉₄ClN₁₃O₁₅: 1367.67].

### Cl₄BH(2,2)IAM-N-HaloTag·4HBr (X═Cl; CoraFluor-2-HaloTag ligand; 56)

Following General Procedure F. substituting CoraFluor-1 ligand (1) with CoraFluor-2 ligand (2). The reaction was run on an 8 mg (5 μmol) scale of CoraFluor-2 ligand (2). Yield=8.9 mg, >95% as a white powder. MS (ESI^{+/-}) m/z (M+H)⁺ 1504.38, m/z (M−H)⁻ 1502.47. [calculated C₆₈H₉₀Cl₅N₁₃O₁₅: 1503.51].

### Br₄BH(2,2)IAM-N-HaloTag·4HBr (X═Br; CoraFluor-3-HaloTag ligand; 57)

Following General Procedure F, substituting CoraFluor-1 ligand (1) with CoraFluor-3 ligand (3). The reaction was run on a 10 mg (6 μmol) scale of CoraFluor-3 ligand (3). Yield=10.8 mg, >95% as a white powder. MS (ESI^{+/-}) m/z (M+H)⁺ 1680.79, m/z (M−H)⁻ 1678.89, [calculated C₆₈H₉₀Br₄ClN₁₃O₁₅: 1679.311.

### BH(2,2)IAM-N-SNAP-tag·4HCl (CoraFluor-1-SNAP ligand; 58)

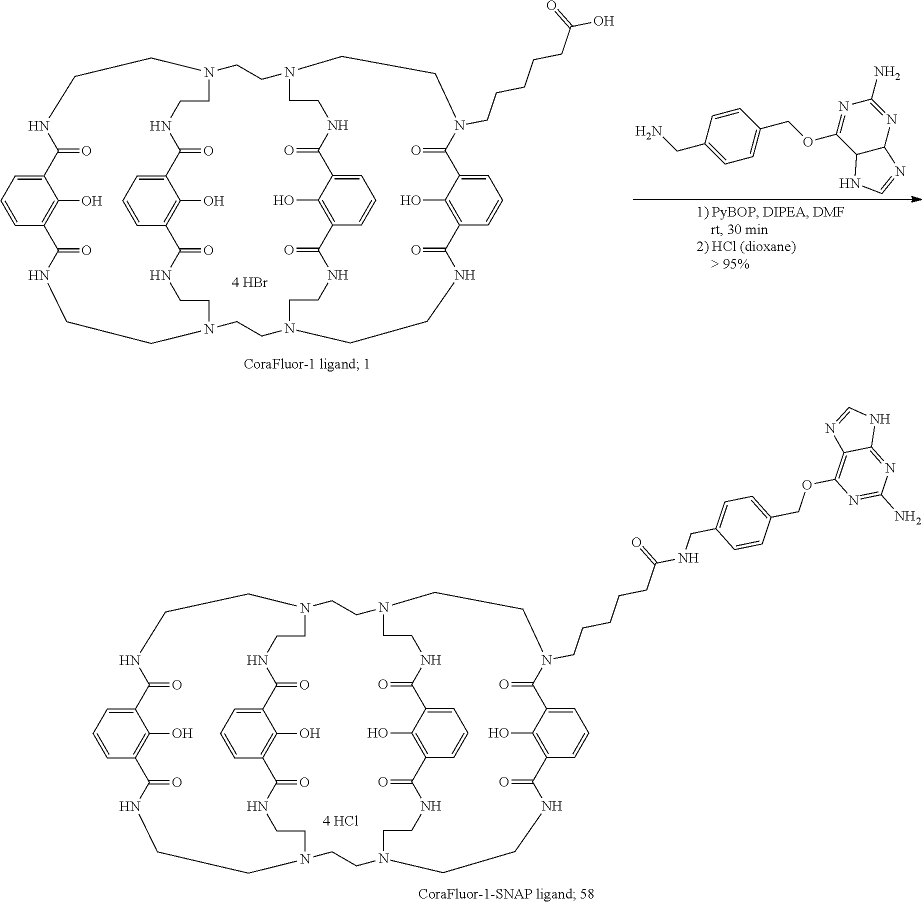 [SMILES: O=C(O)CCCCCN1CCN2CCNC(=O)c3cccc(c3O)C(=O)NCCN(CCNC(=O)c3cccc(c3O)C1=O)CCN1CCNC(=O)c3cccc(c3O)C(=O)NCCN(CCNC(=O)c3cccc(c3O)C(=O)NCC1)CC2 ; NCc1ccc(COC2=NC(N)=NC3N=CNC23)cc1 ; Nc1nc(OCc2ccc(CNC(=O)CCCCCN3CCN4CCNC(=O)c5cccc(c5O)C(=O)NCCN(CCNC(=O)c5cccc(c5O)C3=O)CCN3CCNC(=O)c5cccc(c5O)C(=O)NCCN(CCNC(=O)c5cccc(c5O)C(=O)NCC3)CC4)cc2)c2nc[nH]c2n1]

BH(2,2)IAM-N-hexanoic acid-4HBr (CoraFluor-1 ligand, 1, 10 mg, 6.7 μmol, 1 eq) was dissolved in DMF (400 P L, 10 mM) with DIPEA (23 μL, 17 mg, 135 μmol, 20 eq) then 6-((4-(aminomethyl)benzyl)oxy)-7H-purin-2-amine (2.3 mg, “50 mM” slurry in DMF, 170 μL, 8.4 μmol, 1.25 eq) was added followed by PyBOP (7 mg, 100 mM in DMF, 135 μL, 13.5 μmol, 2 eq) and the reaction mixture was vigorously vortexed for 30 min at room temperature or until all 6-((4-(aminomethyl)benzyl)oxy)-7H-purin-2-amine went into solution. To the reaction mixture was added 4.0 M HCl/dioxane (34 μL, 135 μmol, 20 eq) and the product was precipitated via the addition of 4 mL of Et₂O solution (with sonication). The solid was collected via centrifugation, washed 2×500 μL ACN, and dried overnight in vacuo. Yield=10 mg, >95% as a white powder. MS (ESI^{+/-}) m/z (M+H)⁺ 1415.91, m/z (M−H)⁻ 1413.75. [calculated C₇₁H₈₆N₁₈O₁₄: 1414.66].

### BH(2,2)IAM-N-hexanoate Pfp ester·4HCl (CoraFluor-1-Pfp ligand; 59)

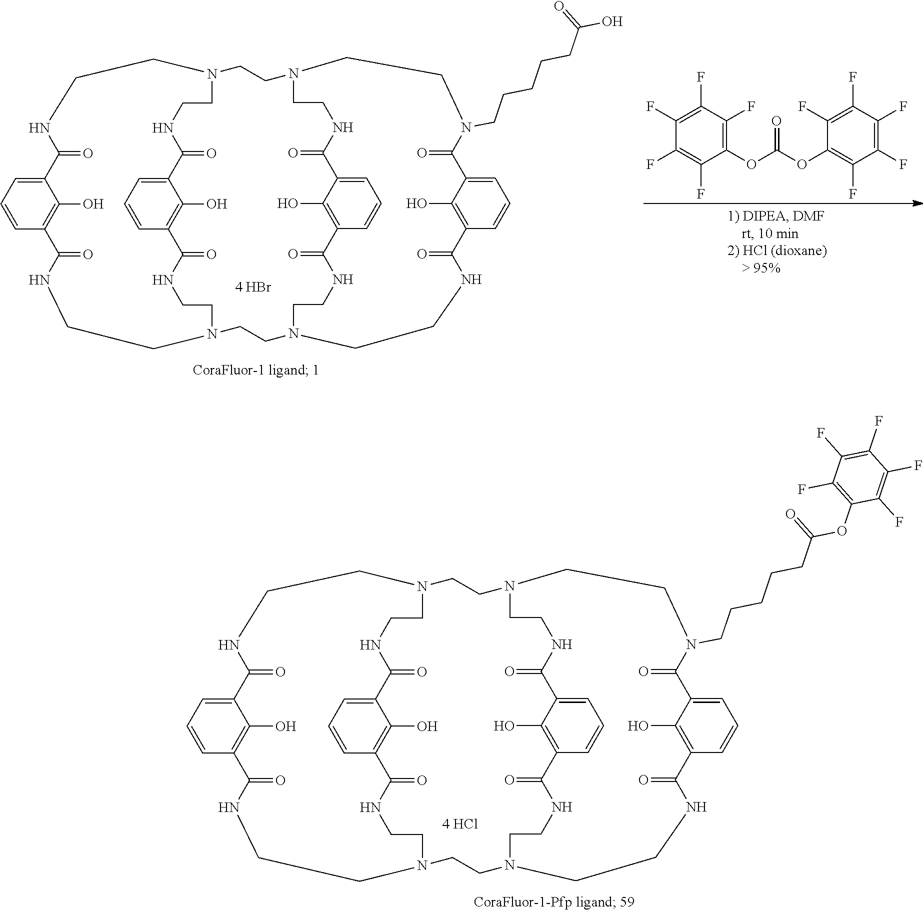 [SMILES: O=C(O)CCCCCN1CCN2CCNC(=O)c3cccc(c3O)C(=O)NCCN(CCNC(=O)c3cccc(c3O)C1=O)CCN1CCNC(=O)c3cccc(c3O)C(=O)NCCN(CCNC(=O)c3cccc(c3O)C(=O)NCC1)CC2 ; O=C(Oc1c(F)c(F)c(F)c(F)c1F)Oc1c(F)c(F)c(F)c(F)c1F ; O=C(CCCCCN1CCN2CCNC(=O)c3cccc(c3O)C(=O)NCCN(CCNC(=O)c3cccc(c3O)C1=O)CCN1CCNC(=O)c3cccc(c3O)C(=O)NCCN(CCNC(=O)c3cccc(c3O)C(=O)NCC1)CC2)Oc1c(F)c(F)c(F)c(F)c1F]

BH(2,2)IAM-N-hexanoic acid-4HBr (CoraFluor-1 ligand, 1, 15.4 mg, 10.4 μmol, 1 eq) was dissolved in DMF (500 μL, ˜10 mM) with DIPEA (36 μL, 27 mg, 207 μmol, 20 eq) then bis(pentafluorophenyl) carbonate (5.1 mg, 100 mM in DMF, 129 μL, 12.9 μmol, 1.25 eq) was added and the reaction mixture was stirred for 10 min at room temperature. To the reaction mixture was added 4.0 M HCl/dioxane (65 μL, 260 μmol, 25 eq) and the product was precipitated via the addition of 3 mL of Et₂O solution (with sonication). The solid was collected via centrifugation, washed 2×500 μL ACN, and dried overnight in vacuo. Yield=15 mg, >95% as a white powder. MS (ESI^{+/-}) m/z (M+H)⁺ 1329.84, m/z (M−H)⁻ 1327.63, [calculated C₆₄H₇₃F₅N₁₂O₁₄: 1328.53].

### 3,3′,3″,3′″-(5,8-bis(2-formamidomethyl)-2,5,8,11-tetraazadodecanedioyl)tetrakis(2-methoxybenzoic acid)·2HCl (X═H; 60)

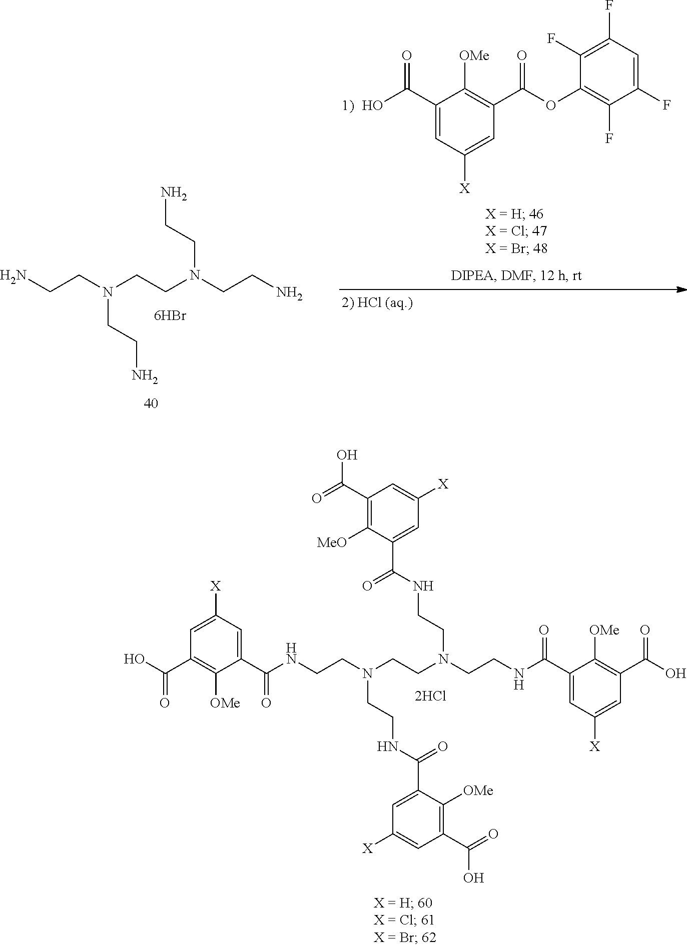

General Procedure G: N1,N1′-(ethane-1,2-diyl)bis(N1-(2-aminoethyl)ethane-1,2-diamine) 6HBr (40, 150 mg, 209 μmol, 1.0 eq) was dissolved in DMF (2 mL) with DIPEA (510 μL, 2.9 mmol, 14 eq) then 2-methoxy-3-((2,3,5,6-tetrafluorophenoxy)carbonyl)benzoic acid (46, 291 mg, 846 μmol, 4.05 eq) was added as a solution in 1 mL DMF. The reaction mixture was stirred for 12 h at room temperature then cooled to 4° C. in an ice bath. Once cooled, aqueous HCl (12.0 M, 522 μL, 6.3 mmol, 30 eq) was added. The reaction was allowed to come to room temperature and was stirred for 15 min to ensure complete acidification of all basic species. The reaction mixture was concentrated in vacuo to an oil, which was first triturated with a mixture of 9:1 Et₂O/ACN (20 mL) to precipitate the crude product as a waxy solid. The solid was allowed to precipitate for 5 min then the mother liquor was carefully decanted and the solid was dried in vacuo for 30 min or until it formed a white foam. The crude product was then triturated 3×5 mL ACN, purified via centrifugation, and dried overnight in vacuo. Yield=205 mg, >95% average yield as a white, fluffy solid. ¹H NMR (400 MHz, D₂O/NaOD) δ 7.49 (t, J=9.1 Hz, 8H), 7.16 (t, J=7.7 Hz, 4H), 3.78 (s, 12H), 3.54 (t, 8H), 2.97-2.73 (m, 12H). ¹³C NMR (101 MHz, D₂O/NaOD) δ 175.50, 169.31, 153.66, 133.72, 130.93, 129.47, 127.65, 124.08, 62.32, 52.26, 50.86, 37.20. MS (ESI^{+/-}) m/z (M+H)⁺ 945.78, m/z (M−H)⁻ 943.68, [calculated C₄₆H₅₂N₆O₁₆: 944.34].

### 3,3′,3″,3′″-(5,8-bis(2-formamidomethyl)-2,5,8,11-tetraazadodecanedioyl)tetrakis(5-chloro-2-methoxybenzoic acid)·2HCl (X═Cl; 61)

Following General Procedure G. substituting 46 with 47. The reaction was run on a 100 mg (139 μmol) scale of 40. Yield=155 mg, >95% as a white, fluffy solid. ¹H NMR (400 MHz, D₂O/NaOD) δ 7.45 (s, 8H), 3.76 (s, 12H), 3.50 (t, J=6.9 Hz, 8H), 2.87-2.74 (m, 12H). ¹¹C NMR (101 MHz, D₂O/NaOD) δ 173.64, 167.72, 152.56, 135.31, 130.61, 129.20, 128.98, 128.47, 62.49, 52.22, 51.13, 37.33. MS (ESI^{+/-}) m/z (M+H)⁺ 1081.40, m/z (M−H)⁻ 1079.37, [calculated C₄₆H₄₈Cl₄N₆O₁₆: 1080.19].

### 3,3′,3″,3′″-(5,8-bis(2-formamidomethyl)-2,5,8,11-tetraazadodecanedioyl)tetrakis(5-bromo-2-methoxybenzoic acid)·2HCl (X═Br; 62)

Following General Procedure G, substituting 46 with 48. The reaction was run on a 175 mg (244 μmol) scale of 40. Yield=312 mg, >95% as a white, fluffy solid. ¹H NMR (400 MHz, D₂O/NaOD) δ 7.61 (s, 4H), 7.59 (s, 4H), 3.77 (s, 12H), 3.50 (t, J=6.3 Hz, 8H), 2.82 (t, J=6.4 Hz, 8H), 2.79 (s, 4H). ¹³C NMR (101 MHz, D₂O/NaOD) δ 173.48, 167.59, 153.08, 135.52, 133.58, 131.91, 129.49, 115.86, 62.41, 52.23, 51.21, 37.36. MS (ESI^{+/-}) m/z (M+H)⁺ 1256.88, m/z (M−H)⁻ 1255.24, [calculated C₄₆H₄₈Br₄N₆O₁₆: 1255.99].

### Me₄BH(2,2)IAM (X═H; 63)

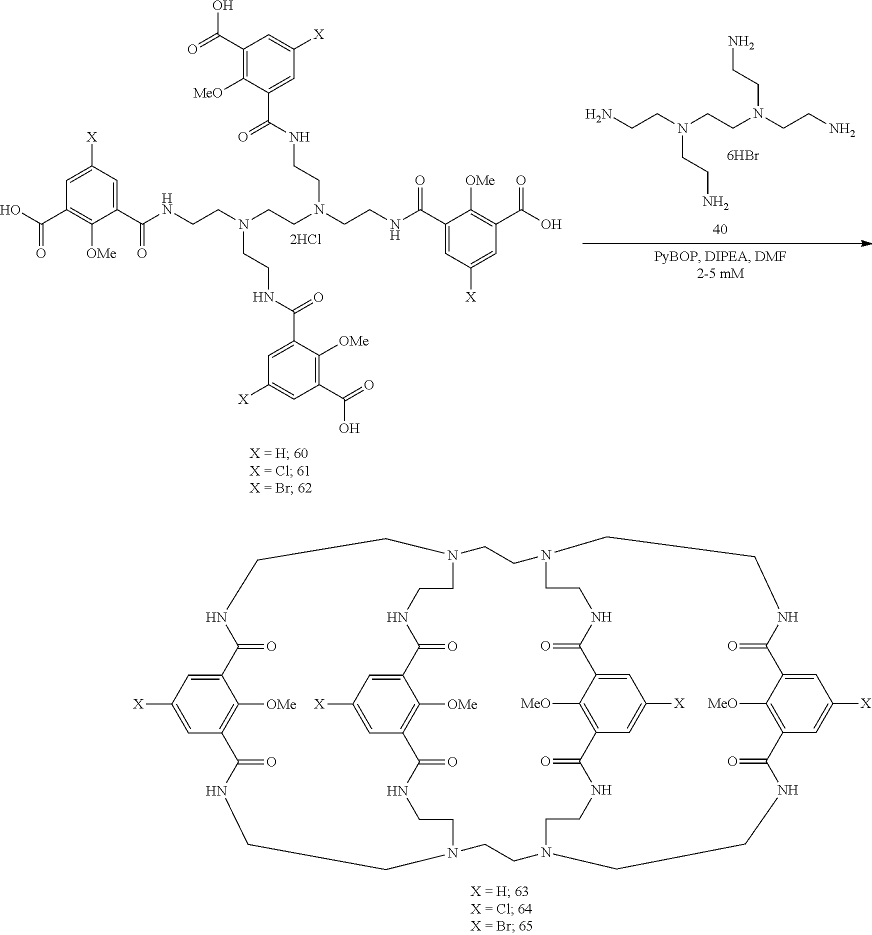

General Procedure H: 3,3′,3″,3′″-(5,8-bis(2-formamidomethyl)-2,5,8,11-tetraazadodecanedioyl)tetrakis(2-methoxybenzoic acid) 2HCl (60, 142 mg, 140 μmol, 1.05 eq) was dissolved in DMF (30 mL, ˜3 mM) with DIPEA (700 μL, 519 mg, 4 mmol, 30 eq) then N1,N1′-(ethane-1,2-diyl)bis(NI-(2-aminoethyl)ethane-1,2-diamine)·6HBr (40, 95 mg, 133 μmol, 1 eq) was added to create a slightly cloudy solution. In a separate container, PyBOP (416 mg, 799 μmol, 6 eq) was dissolved in DMF (13 mL, −60 mM). The solution of PyBOP/DMF was added dropwise to the solution of 40 and 60 in DMF via a syringe pump (5 mL/h or 1 drop every 3-4 sec) over approximately 2.5 h with vigorous stirring. Upon complete addition of the PyBOP solution, the reaction mixture became a clear, slightly pale-yellow solution. The reaction mixture was allowed to stir at room temperature for 30 min then the mixture was concentrated in vacuo to an oil (water bath temp 40-50° C.). The crude product was precipitated first as a waxy solid with 9:1 Et₂O/ACN (20 mL). The product was allowed to precipitate for 5 min then the mother liquor was carefully decanted, and the trituration process repeated twice more. The crude solid was dried in vacuo for 30 min or until it became a white foam. The solid was purified via flash chromatography (λ225 nm, 285 nm; 7% MeOH/DCM+1% NH₄OH). Yield=99 mg, 67% average yield as a white, crystalline solid. ¹H NMR (400 MHz, CDCl₃) δ 7.67 (d, J=7.6 Hz, 8H), 7.61 (t, 8H), 7.01 (t, J=7.6 Hz, 4H), 3.66 (s, 12H), 3.61-3.53 (m, 8H), 3.52-3.42 (m, 8H), 2.85-2.78 (m, 8H), 2.74-2.66 (m, 16H). ¹³C NMR (101 MHz, CDCl₃) δ 165.81, 155.72, 133.39, 128.32, 124.88, 63.23, 53.74, 51.75, 38.26. MS (ESI^{+/-}) m/z (M+H)⁺ 1105.83, m/z (M−H)⁻ 1103.71, [calculated C₅₆H₇₂N₁₂O₁₂: 1104.54].

### Me₄Cl₄BH(2,2)IAM (X═Cl; 64)

Following General Procedure H. substituting 60 with 61. The reaction was run on a 94 mg (130 μmol) scale of 40. Flash chromatography conditions λ225 nm, 295 nm: 6% MeOH/DCM+1% NH₄OH. Yield=88 mg, 61% average yield as a white, crystalline solid. ¹H NMR (400 MHz, DMSO-d₆) δ 8.25 (t, J=4.7 Hz, 8H), 7.56 (s, 8H), 3.70 (s, 12H), 3.57-3.44 (m, 8H), 3.32-3.26 (m, 8H), 2.83-2.72 (m, 24H). ¹³C NMR (101 MHz, DMSO-d₆) δ 163.85, 153.57, 131.40, 130.53, 127.63, 62.61, 52.02, 48.59, 36.84. MS (ESI^{+/-}) m/z (M+H)⁺ 1241.65, m/z (M−H)⁻ 1239.76, [calculated C₅₆H₆₈C₁₄N₁₂O₁₂: 1240.38].

### Me₄Br₄BH(2,2)IAM (X═Br; 65)

Following General Procedure H, substituting 60 with 62. The reaction was run on a 73 mg (102 μmol) scale of 40. Flash chromatography conditions: λ225 nm, 295 nm; 5% MeOH/DCM+1% NH₄OH. Yield=88 mg. 61% as a white, crystalline solid. ¹H NMR (400 MHz, DMSO-d₆) δ 8.25 (t, J=5.3 Hz, 8H), 7.68 (s, 8H), 3.70 (s, 12H), 3.55-3.45 (m, 8H), 3.32-3.23 (m, 8H), 2.81-2.71 (m, 24H). ¹³C NMR (101 MHz, DMSO-d₆) δ 163.76, 154.06, 133.43, 131.67, 115.44, 62.53, 52.04, 48.52, 36.84. MS (ESI^{+/-}) m/z (M+H)⁺ 1417.42, m/z (M−H)⁻ 1415.62. [calculated C₅₆H₆₈Br₄N₁₂O₁₂: 1416.18].

### BH(2,2)IAM·4HBr (X═H; 66)

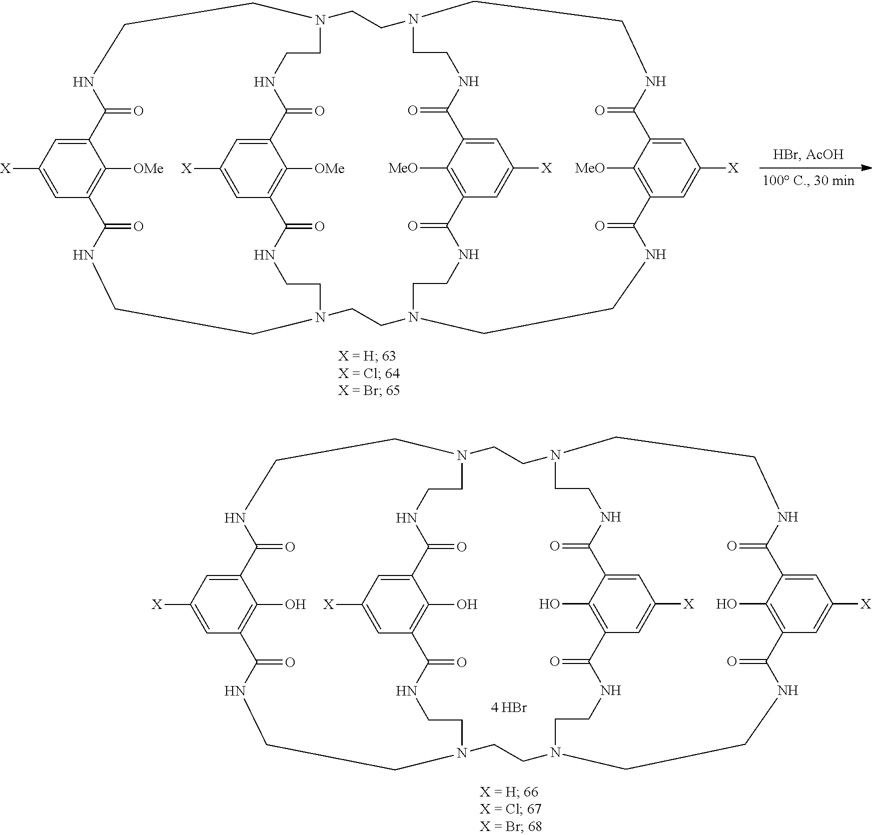

General Procedure I: Me₄BH(2,2)IAM (63, 14 mg, 13 μmol, 1 eq) was dissolved in AcOH (1.2 mL, ˜10 mM) then 48% wt aqueous HBr (29 μL, 252 μmol, eq) was added and the reaction mixture was equipped with a reflux condenser and heated to 100° C. for 30 min with good stirring. After the deprotection period, the crude solid precipitated out of solution. Additional solid was precipitated via the addition of 1 mL Et₂O and the product was purified via centrifugation, washed 2×1 mL Et₂O, and dried in vacuo. Yield=17 mg, >95% as a white powder. ¹H NMR (400 MHz, D₂O/NaOD) 5 7.63 (d, J=7.7 Hz, 6H), 7.47 (d, J=7.7 Hz, 2H), 6.40 (t, J=7.7 Hz, 3H), 6.23 (t, J=7.6 Hz, 1H), 3.46-3.14 (m, 18H), 2.87-2.70 (m, 22H). ¹C NMR (101 MHz, D₂O/NaOD) δ 170.16, 170.00, 169.87, 133.91, 120.29, 112.01, 52.00, 51.01, 37.06. MS (ESI^{+/-}) m/z (M+H)⁺ 1049.69, m/z (M−H)⁻ 1047.57, [calculated C₅₂H₆₄N₁₂O₂₁: 1048.48].

### Cl₄BH(2,2)IAM·4HBr (X═Cl; 67)

Following General Procedure I, substituting 63 with 64. The reaction was run on a 15 mg (12 μmol) scale of 64. Yield=18 mg, >95% as a white powder. ¹H NMR (400 MHz, D₂O/NaOD) δ 7.50 (s, 6H), 7.31 (s, 2H), 3.45-3.09 (m, 18H), 2.84-2.63 (m, 22H). ¹³C NMR (101 MHz, D₂O/NaOD) δ 168.94, 168.67, 132.93, 132.12, 121.93, 121.56, 115.80, 51.99, 51.21, 50.46, 37.86, 37.27. MS (ESI^{+/-}) m/z (M+H)⁺ 1185.50, m/z (M−H)⁻ 1183.43. [calculated C₅₂H₆₀Cl₄N₁₂O₁₂: 1184.32].

### Br₄BH(2,2)IAM·4HBr (X═Br; 68)

Following General Procedure I, substituting 63 with 65. The reaction was run on a 15 mg (11 μmol) scale of 65. Yield=17.5 mg, >95% as a white powder. ¹H NMR (400 MHz, D₂O/NaOD) δ 7.72 (s, 8H), 3.54-3.42 (m, 8H), 3.33-3.15 (m, 8H), 2.92-2.73 (m, 24H). ¹³C NMR (101 MHz, D₂O/NaOD) δ 168.90, 168.59, 135.91, 122.15, 102.66, 52,04, 50.55, 37.29. MS (ESI^{+/-}) m/z (M+H)⁺ 1361.32, m/z (M−H)⁻ 1359.17, [calculated C₅₂H₆₀Br₄N₁₂O₁₂: 1360.12].

### 4-chloro-2,6-dimethylanisole (69)

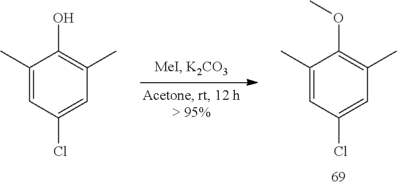

4-chloro-2,6-dimethylphenol (4.5 g, 29 mmol, 1 eq) was dissolved in acetone (100 mL) then K₂CO₃ (9.9 g, 72 mmol, 2.5 eq) was added followed by Mel (2.7 mL, 6.1 g, 43 mmol, 1.5 eq) and the resulting suspension was stirred for 3 h at room temperature. After, an additional 8 g K₂CO₃ (58 mmol, 2 eq) and 2.7 mL Mel (6.1 g, 43 mmol, 1.5 eq) was added and the reaction was stirred for 12 h at room temperature. The reaction mixture was concentrated to dryness in vacuo and the resulting crude solid was partitioned between 200 mL EtOAc and 200 mL H₂O. The organic layer was separated and washed once more with 200 mL H₂O, 1×200 mL saturated brine solution. The organic layer was then dried over Na₂SO₄, filtered, and concentrated in vacuo overnight. Yield=4.85 g, >95% as a slightly yellow oil. ¹H NMR (400 MHz, CDCl₆) δ 6.99 (s, 2H), 3.69 (s, 3H), 2.25 (s, 6H). ¹³C NMR (101 MHz, CDCl₃) δ 155.70, 132.74, 128.56, 128.55, 77.16, 59.95, 16.14.

### Terbium Loading

Terbium ligands (5-20 mg) were dissolved in MeOH to a concentration of ˜1 mM with an excess of pyridine (50 eq) then TbCl₃·6H₂O (2 eq) was added as a 100 mM solution in MeOH. The reaction mixtures were stirred for 10 min at room temperature, at which point LCMS analysis indicated quantitative Tb complex formation. The reaction mixtures were concentrated to dryness in vacuo then the complexes were precipitated via the addition of 500 μL ACN (with sonication). The crude products were collected via centrifugation, washed 2×500 μL ACN, and dried overnight in vacuo. Yields for Tb complexes were >90% in all cases.

The terbium complexes were further characterized by LCMS. 12, MS (ESI^{+/-}) m/z (M+H)⁺ 1205.64, m/z (M−H)⁻ 1203.60, [calculated C₅₂H₆₁N₁₂O₁₂Tb: 1204.38]. 13. MS (ESI^{+/-}) m/z (M+H)⁺ 1341.54, m/z (M−H)⁻ 1339.32, [calculated C₅₂H₅₇Cl₄N₁₂O₁₂Tb: 1340.22]. 14, MS (ESI^{+/-}) m/z (M+H)⁺ 1517.24, m/z (M−H)⁻ 1515.22, [calculated C₅₂H₅₇Br₄N₁₂O₁₂Tb: 1516.02]. CoraFluor-1, MS (ESI^{+/-}) m/z (M+H)⁺ 1319.76, m/z (M−H)⁻ 1317.61. [calculated C₅₈H₇₁N₁₂O₁₄Tb: 1318.45]. CoraFluor-1-Halo, MS (ESI^{+/-}) m/z (M+H)⁺ 1524.83, m/z (M−H)⁻ 1522.56, [calculated C₆₈H₉₁ClN₁₃O₁₅Tb: 1523.57]. CoraFluor-1-SNAP, MS (ESI^{+/-}) m/z (M+H)⁺ 1571.77, m/z (M−H) 1569.71, [calculated C₇₁H₅₃N₁₈O₁₄Tb: 1570.56]. CoraFluor-1-Pfp, MS (ESI^{+/-}) m/z (M+H)⁺ 1485.28, m/z (M−H)⁻ 1483.52, [calculated C₆₄H₇₀F₅N₁₂O₁₄Tb: 1484.43]. CoraFluor-2-Halo, MS (ESI⁺) m/z (M+H)⁺ 1660.69, m/z (M−H)⁻ 1658.58, [calculated C₆₈H₈₇Cl₅N₁₃O₁₅Tb: 1659.41]. CoraFluor-3-Halo, MS (ESI^{+/-}) m/z (M+H)⁺ 1836.81. m/z (M−H)⁻ 1834.49, [calculated C₆₈H₈₇Br₄ClN₁₃O₁₅Tb: 1835.21]. CoraFluor-1-KL9. MS (ESI^{+/-}) m/z (M+2H)²⁺ 1176.28, m/z (M−2H)²⁻ 1174.79, [calculated C₁₀₄H₁₃₉N₂₂O₃₁Tb: 2350.92].

### N^{α}-acetyl-LDEETGEFL-CONH₂ (Ac-KL9; 26)

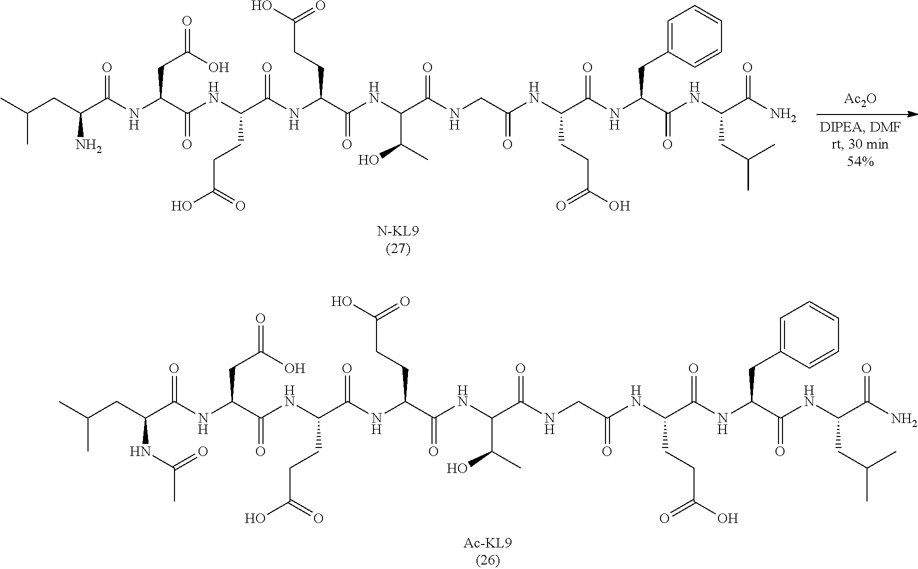

N-KL9 (27, 4 mg, 3.8 μmol, 1 eq) was dissolved in DMF (400 μL) with DIPEA (7.3 mg, 10 μL, 56 μmol, 15 eq) then acetic anhydride (0.4 mg, 200 mM in DMF. 20 μL, 4 μmol, 1.05 eq) was added and the reaction was briefly vortexed and so allowed to stand at room temperature for 30 min. The reaction mixture was acidified with 4.0 M HCl/dioxane (38 μL, 150 μmol, 40 eq) and the crude product was precipitated via the addition of 5 mL Et₂O. The mother liquor was carefully decanted and the solid was further triturated with 500 μL ACN, purified via centrifugation, washed 2×500 μL ACN, and dried overnight in vacuo. Yield=2.2 mg, 54% as a white solid. MS (ESI^{+/-}) m/z (M+H)⁺ 1093.76, m/z (M−H)⁻ 1091.52, [calculated C₄₈H₇₂N₁₀O₁₉: 1092.50].

### N^{α}-CoraFluor-1-LDEETGEFL-CONH₂ ligand (CoraFluor-1-KL9 ligand; 70)

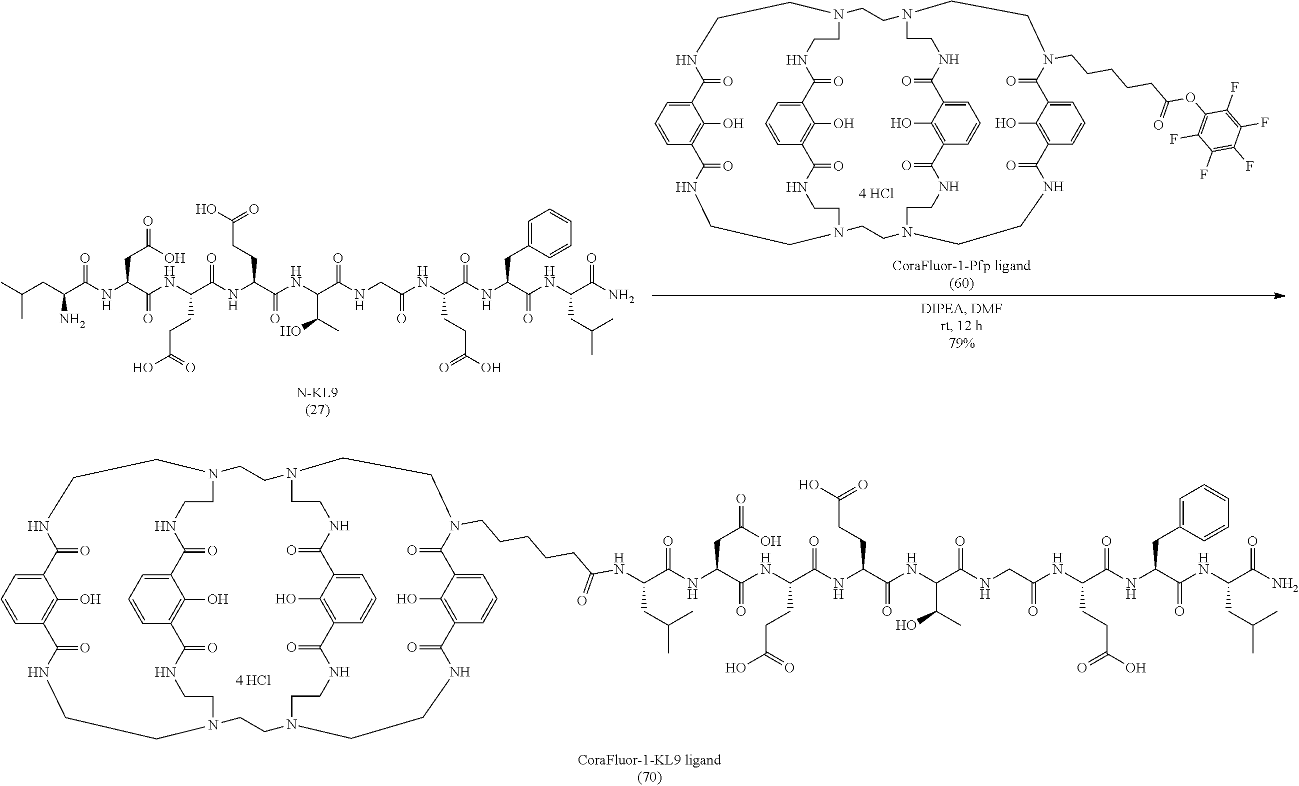

N-KL9 (27, 4 mg, 3.89 μmol, 1.05 eq) was dissolved in DMF (500 μL) with DIPEA (14.1 mg, 19 μL, 109 μmol, 30 eq) then CoraFluor-1-Pfp ligand (59, 5.4 mg, 3.6 μmol, 1 eq) was added and the reaction was briefly vortexed and allowed to stand at room temperature for 12 h. The reaction mixture was acidified with 4.0 M HCl/dioxane (54 μL, 217 μmol, 60 eq) and the crude product was precipitated via the addition of 2 mL Et₂O. The mother liquor was carefully decanted and the solid was further triturated with 500 μL ACN, purified via centrifugation, washed 2×500 μL ACN, and dried overnight in vacuo. Yield=6.6 mg, 78% as a white solid. MS (ESI^{+/-}) m/z (M+2H)²⁺ 1098.50, m/z (M−2H)²⁻ 1096.73, [calculated C₁₀₄H₁₄₂N₂₂O₃₁: 2195.02].

### Perfluorophenyl (4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicene-4a(2H)-carboxylate—CDDO-Pfp—(71)

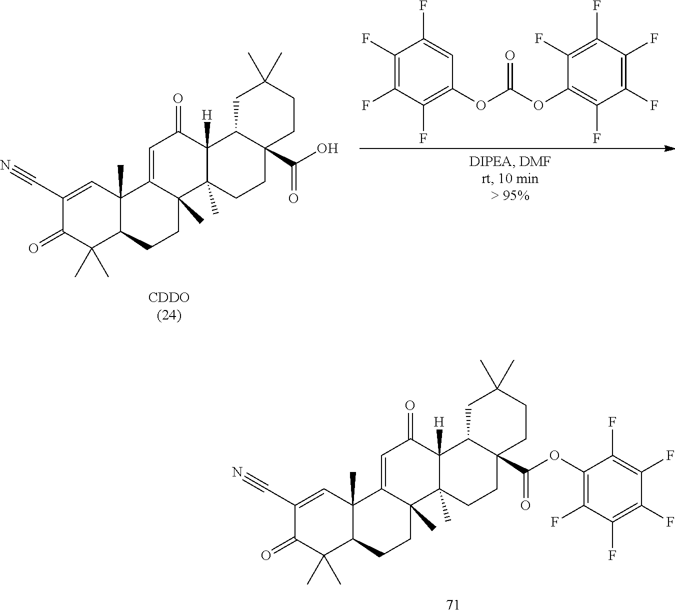 [SMILES: CC1(C)CC[C@]2(C(=O)O)CC[C@]3(C)[C@H](C(=O)C=C4[C@@]5(C)C=C(C#N)C(=O)C(C)(C)[C@@H]5CC[C@]43C)[C@@H]2C1 ; O=C(Oc1cc(F)c(F)c(F)c1F)Oc1c(F)c(F)c(F)c(F)c1F ; CC1(C)CC[C@]2(C(=O)Oc3c(F)c(F)c(F)c(F)c3F)CC[C@]3(C)[C@H](C(=O)C=C4[C@@]5(C)C=C(C#N)C(=O)C(C)(C)[C@@H]5CC[C@]43C)[C@@H]2C1]

(4aS,6aR,6bS,8aR,12aS,14aR,14bS)-11-cyano-2,2,6a,6b,9,9,12a-heptamethyl-10,14.dioxo-1,3,4,5,6,6a,6b,7,8,8a,9,10,12a,14,14a,14b-hexadecahydropicene-4a(2H)-carboxylic acid (CDDO (24), 300 mg, 610 μmol, 1 eq) was dissolved in DMF (2 mL) with DIPEA (316 mg, 425 IL, 2.44 mmol, 4 eq) then bis(pentafluorophenyl) carbonate (265 mg, 671 μmol, 1.1 eq) was added and the reaction mixture was stirred for 10 min at room temperature. The reaction mixture was diluted into 100 mL EtOAc and the organic layer was washed 2×100 mL 0.2 N HCl, 2×100 mL 0.2 N NaHCO₃, and 1× saturated brine solution. The organic layer was dried over Na₂SO₄, filtered, and concentrated in vacuo to give the compound without further purification. Yield=400 mg, >95% as a white, crystalline solid. ¹H NMR (400 MHz, CDCl₃) 5 8.04 (s, 1H), 6.00 (s, 1H), 3.18 (dt, J=13.7, 4.1 Hz, 1H), 3.03 (d, J=4.6 Hz, 1H), 2.21-2.00 (m, 2H), 1.92-1.71 (m, 7H), 1.68-1.55 (m, 3H), 1.50 (s, 3H), 1.45-1.39 (m, 1H), 1.38 (s, 3H), 1.35-1.28 (m, 2H), 1.26 (s, 3H), 1.18 (s, 3H), 1.06 (s, 3H), 1.04 (s, 3H), 0.94 (s, 3H). ¹³C NMR (101 MHz, CDCl₃) δ 198.43, 196.65, 174.15, 169.12, 165.74, 142.65, 140.01, 139.24, 136.83, 125.34, 124.13, 114.80, 114.47, 49.92, 48.51, 47.87. 45.94, 45.18, 42.71, 42.30, 35.76, 34.43, 33.24, 32.98, 31.89, 31.85, 30.76, 28.07, 27.15, 26.85, 24.77, 23.06, 22.86, 21.76, 21.70, 18.35.

### 1-((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-1-oxo-6,9,12-trioxa-2-azapentadecan-15-aminium formate—CDDO-Amine—(72)

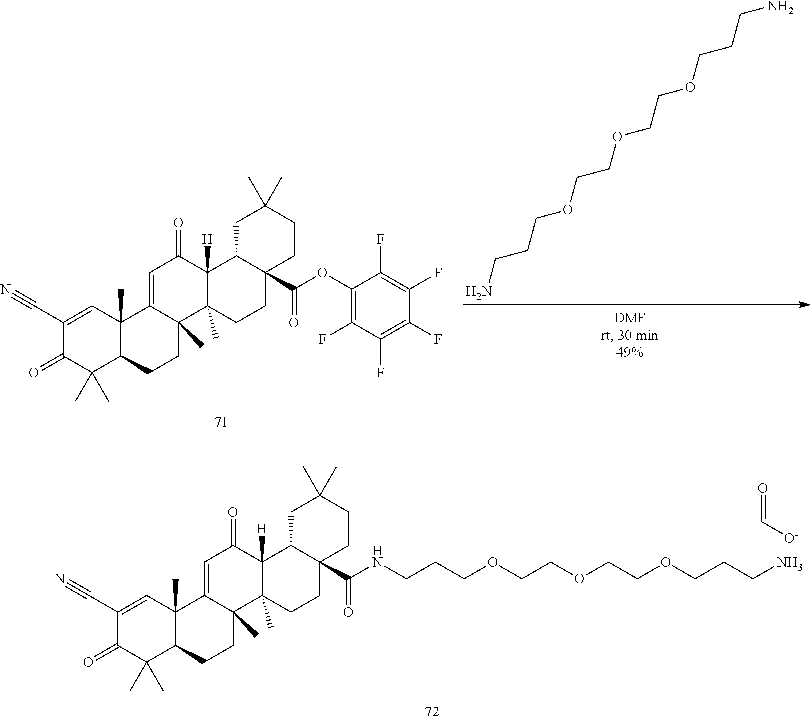

Perfluorophenyl (4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicene-4a(2H)-carboxylate (71, 100 mg, 152 μmol, 1 eq) was dissolved in DMF (1 mL) then 4,7,10-trioxa-1,13-tridecanediamine (335 mg, 333 μL, 1.52 mmol, 10 eq) was added and the reaction was allowed to stand at room temperature for 30 min. The reaction mixture was directly purified via reverse phase flash chromatography (λ220 nm, 263 nm; gradient: 95% H₂O/5% ACN+0.1% formic acid for 2 CV, 95% H₂O/5% ACN+0.1% formic acid to 40% H₂O/60% ACN+0.1% formic acid over 10 CV, 40% H₂O/60% ACN+0.1% formic acid to 100% ACN+0.1% formic acid over 1 CV, 100% ACN+0.1% formic acid for 3 CV). Yield=55 mg, 49% as an off-white, fluffy solid. ¹H NMR (400 MHz, DMSO-d₆) δ 8.39 (s, 1H), 5.67 (s, 1H), 3.55-3.41 (m, 10H), 3.38 (t, J=6.1 Hz, 3H), 3.20-3.03 (m, 2H), 3.03-2.91 (m, 2H), 2.88-2.72 (m, 3H), 2.34 (d, J=14.6 Hz, 1H), 2.14 (d, J=14.5 Hz, 1H), 1.87 (t, J=12.8 Hz, 1H), 1.75 (p, J=6.3 Hz, 2H), 1.68-1.50 (m, 9H), 1.49-1.19 (m, 7H), 1.18-1.11 (m, 6H), 1.09 (s, 4H), 1.01 (s, 3H), 0.93 (s, 3H), 0.88 (s, 3H), 0.85 (s, 3H). ¹³C NMR (101 MHz, DMSO-d₆) δ 199.40, 176.49, 175.92, 165.47, 123.42, 69.74, 69.69, 69.55, 69.48, 68.48, 68.11. 67.52, 48.55, 48.22, 45.44, 45.05, 41.21, 38.22, 36.71, 36.26, 35.55, 34.21, 33.29, 33.14, 30.85, 30.79, 30.29, 29.86, 29.58, 27.83, 27.46, 23.70, 23.18, 22.92, 21.89, 21.10, 19.55, 18.51. MS (ESI^{+/-}) m/z (M+H)⁺ 694.47, m/z (M−H)⁻ 692.71, [calculated C₄₁H₆₃N₃O₆: 693.47].

### 5-(3-(1-((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-1-oxo-6,9,12-trioxa-2-azapentadecan-15-yl)thioureido)-2-(6-hydroxy-3-oxo-3H-xanthen-9-yl)benzoic acid (CDDO-FITC; 19)

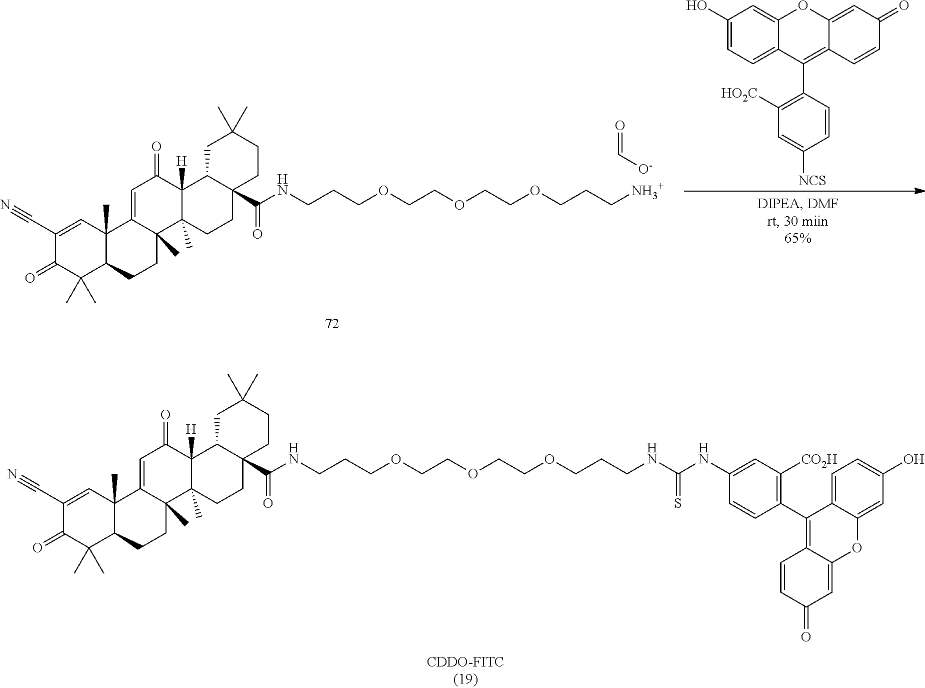

1-((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-1-oxo-6,9,12-trioxa-2-azapentadecan-15-aminium formate (72, 7.0 mg, 9.5 μmol, 1.05 eq) was dissolved in DMF (500 μL) with DIPEA (5.8 mg, 7.8 μL, 45 μmol, 5 eq) then 5(6)-fluorescein isothiocyanate (5(6)-FITC, 3.5 mg, 9.0 μmol, 1 eq) was added and the reaction was allowed to stand at room temperature in the dark for 30 min. The reaction mixture was directly purified via reverse phase flash chromatography (a 225 nm, 275 nm; gradient: 95% H₂O/5% ACN+0.1% formic acid for 2 CV. 95% H₂O/5% ACN+0.1% formic acid to 100% ACN+0.1% formic acid over 18 CV, 100% ACN+0.1% formic acid for 3 CV). Yield=6.4 mg, 65% as an orange film. ¹H NMR (400 MHz, DMSO-d₆) δ 10.12 (s, 2H), 9.94 (s, 1H), 8.64 (s, 1H), 8.22 (s, 1H), 8.10 (s, 1H), 7.73 (d. J=6.0 Hz, 1H), 7.67 (d, J=6.1 Hz, 1H), 7.17 (d, J=8.2 Hz, 1H), 6.67 (s, 2H), 6.58 (q, J=8.6 Hz, 4H), 6.19 (s, 1H), 3.62-3.43 (m, 12H), 3.39 (t, J=6.0 Hz, 4H), 3.22-3.13 (m, 1H), 3.12-2.98 (m, 2H), 2.85 (d, J=12.8 Hz, 1H), 1.95-1.72 (m, 5H), 1.71-1.51 (m, 6H), 1.47-1.37 (m, 5H), 1.36-1.24 (m, 2H), 1.22 (s, 3H), 1.15 (s, 3H), 1.12-0.99 (m, 5H), 0.92 (s, 3H), 0.90-0.79 (m, 6H). ¹³C NMR (101 MHz, DMSO) δ 199.26, 197.33, 176.43, 168.96, 168.53, 168.35, 159.60, 151.93, 141.28, 129.04, 124.11, 123.39, 115.12, 112.80, 112.64, 109.77, 102.25, 69.79, 69.76, 69.59, 68.45, 68.20, 48.70. 46.21, 45.34, 44.37, 42.55, 41.49, 36.33, 35.36, 34.15, 33.27, 33.08, 31.17, 30.75, 30.24, 29.57, 28.58, 27.28, 26.11, 25.86, 24.34, 23.16, 21.83, 21.31, 21.13, 17.46. MS (ESI^{+/-}) m/z (M+H)⁺ 1083.80, m/z (M−H)⁻ 1081.58, [calculated C₆₂H₇₄N₄O₁₁S: 1082.51].

### (4aS,6aR,6bS,8aR,12aS,14aR,14bS)-N-(1-(4-(4-chlorophenyl)-2,3,9-trimethyl-6H-thieno[3,2-f][1,2,4]triazolo[4,3-a][1,4]diazepin-6-yl)-2-oxo-7,10,13-trioxa-3-azahexadecan-16-yl)-11-cyano-2,2,6a,6b,9,9,12a-heptamethyl-10,14-dioxo-1,3,4,5,6,6a,6b,7,8,8a,9,10,12a,14,14a,14b-hexadecahydropicene-4a(2H)-carboxamide (CDDO-JQ1; 23)

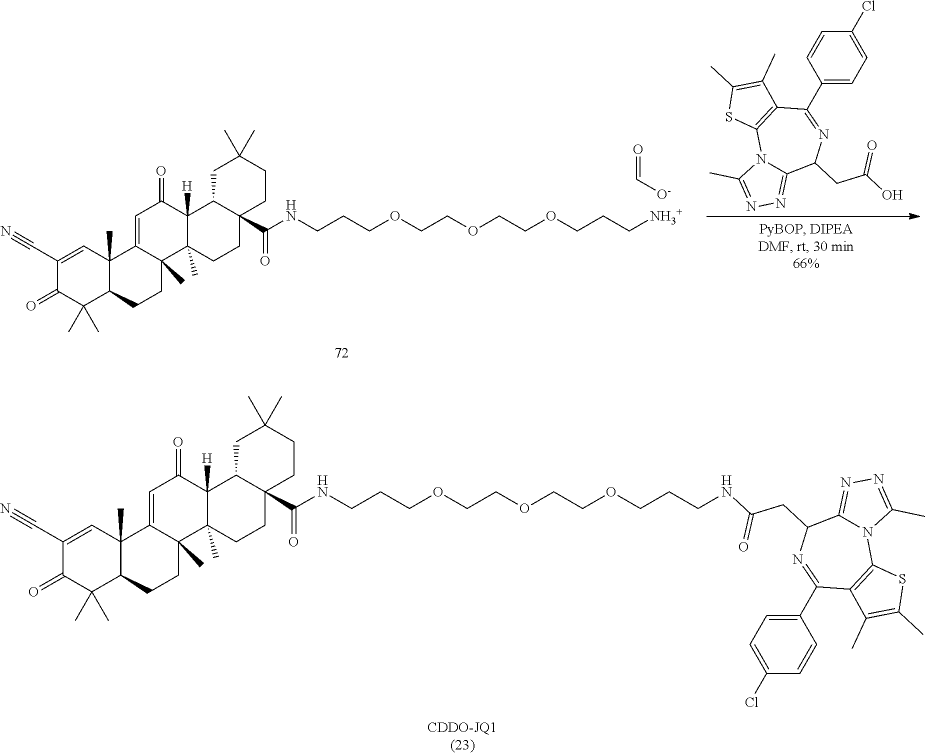

JQ1-Acid (8.0 mg, 14 μmol, 1 eq) was dissolved in DMF (500 L) with DIPEA (49.0 μL, 36.4 mg, 282 μmol, 20 eq) then PyBOP (7.3 mg, 14.1 μmol, 1 eq) was added and the activation reaction was allowed to proceed at room temperature for 10 min. After, 1-((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-1-oxo-6,9,12-trioxa-2-azapentadecan-15-aminium formate (72, 12.5 mg, 16.9 μmol, 1.2 eq) was added and the reaction was allowed to stand at room temperature for 30 min. The reaction mixture was directly purified via reverse phase flash chromatography (λ225 nm, 250 nm; gradient: 95% H₂O/5% ACN+0.1% formic acid for 2 CV, 95% H₂O/5% ACN+0.1% formic acid to 100% ACN+0.1% formic acid over 18 CV. 100% ACN+0.1% formic acid for 3 CV). Yield=7.4 mg, 66% as a slightly yellow solid. ¹H NMR (400 MHz, CDCl₃) δ 8.01 (s, 1H), 7.81-7.62 (m, 1H), 7.47 (d, J=8.1 Hz, 2H), 7.36 (d, J=8.1 Hz, 2H), 6.93-6.76 (m, 1H), 6.11-5.91 (m, 1H), 5.03-4.80 (m, 1H), 3.71-3.52 (m, 12H), 3.48-3.26 (m, 4H), 3.06 (d, J=4.0 Hz, 1H), 2.89 (s, 3H), 2.44 (s, 3H), 1.97-1.85 (m, 3H), 1.82-1.71 (m, 6H), 1.68 (s, 3H), 1.60-1.43 (m, 6H), 1.39 (s, 3H), 1.32 (s, 3H), 1.29-1.26 (m, 2H), 1.25-1.18 (m, 6H), 1.17-1.14 (m, 2H), 1.09 (s, 3H), 1.02 (s, 3H), 0.98 (s, 3H), 0.88 (s, 3H). ¹³C NMR (101 MHz, CDCl₃) δ 199.78, 196.81, 177.22, 169.16, 166.43, 131.34, 130.22, 129.08, 124.07, 114.62, 114.20, 81.64, 70.58, 70.49, 70.43, 70.42, 70.30, 69.54, 53.33, 49.62, 47.57, 46.49, 46.05, 45.05, 42.74, 42.33, 38.20, 37.76, 36.26, 34.76, 34.07, 33.50, 31.91, 31.77, 30.76, 29.82, 29.36, 29.15, 28.27, 27.94, 27.00, 26.60, 24.95, 23.36, 22.76, 21.95, 21.66, 18.32, 14.62, 13.45, 11.63. MS (ESI⁺) m/z (M+H)⁺ 1076.86, m/z (M−H)⁻ 1074.16. [calculated C₆₀H₇₈ClN₇O₇S: 1075.54].

### Example 7—Quantification of Protein of Interest

It is often desirable to quantify the abundance of a target POI, e.g. to assess the efficiency of recombinant protein expression or the response to small molecule treatment. A POI can be secreted, be present on the cell surface or be intracellular. For non-secreted POI, and particularly for intracellular POIs, the quantification is often performed using lysed or permeabilized cell. However, in certain applications it might be desirable to directly quantify the abundance of a POI in live cells. Amongst other applications this is of particular interest for target protein degradation.

In recent years, the development of small molecules that mediate the degradation of specific target proteins as a novel therapeutic approach for a wide range of human diseases has attracted great attention from both academia and industry. While other modes of small molecule induced protein degradation have been discovered, most compounds, commonly referred to as “degraders”, act as “molecular glues” that facilitate or stabilize binding of the protein target of interest to a ubiquitin ligase complex. If the target protein is positioned appropriately to be recognized as substrate, the E3-ligase will ubiquitinylate the bound protein resulting in its consecutive elimination by proteasomal degradation. Although some small molecules, including existing drugs, have been discovered serendipitously to function as degraders, the rational design employs a modular approach by tethering a ligand for the target protein to a small molecule binder of an E3 ligase complex using a flexible linker. The resulting heterobifunctional small-molecules are known as proteolysis-targeting chimeras (PROTACs).

Critical for the development of PROTACs is the ability to characterize the binding affinity and kinetics of the bifunctional molecule to either of its binding partners as well as the simultaneous binding to both binding partners that result in the formation of the ternary complex of E3-ligase, PROTAC and target protein.

Furthermore, the ability to readily and accurately quantify the expression levels of the POI in high throughput is critical to thoroughly assess the time and concentration dependent action of PROTACs. Several methods have been developed. Western Blot Analysis is considered the gold standard. While Western Blot Analysis allows for the quantification of wild-type protein it has several disadvantages including relatively low throughput, limited dynamic range, lengthy procures, and material amount. Although automated methods have been developed that significantly increase throughput, they do require specialized equipment. ELISAs have been developed as alternatives. While providing a large dynamic range and good sensitivity, this method generally requires matched antibody pairs for the quantification of wild-type POI. More recently, the development of split-luciferase based approaches such as Promega's HiBiT/LgBiT system have been developed. However, these approaches require the genetic modification of the POI to install an 11-amino acid tag.

This example demonstrates a simplified approach using TR-FRET technology. Combination of 1) a TR-FRET donor-labeled antibody that is direct against an endogenous protein sequence, or a specific epitope tag is used to install a TR-FRET donor, and 2) a small molecule ligand (tracer) that binds the POI with sufficiently high affinity is labeled with a suitable fluorescent molecule to function as TR-FRET acceptor enables direct quantification of the target protein if both antibody and tracer ligand are used at concentration in higher than the contraction of the POI.

Since PROTACs are based on linker-functionalized small molecule ligands that have been validated to bind the target POI, they provide straightforward access to suitable TR-FRET tracers.

Another experimental approach is the direct quantification of HaloTag (Promega) fusion proteins in cellular lysates (see attached manuscript draft). HaloTag is a self-labeling protein domain that specifically and covalently reacts with a HaloTag ligand. This allows to selectively and efficiently tag HaloTag fusion proteins with functionalized small molecule ligands. In addition, HaloTag proteins can specifically targeted with specific nanobodies such as HaloTrap (ChromoTek). The combination of orthogonally acceptor/donor labeled HaloTag ligand and HaloTrap enables the direct quantification of HaloTag fusion proteins. Similar approaches are obvious for other self-labeling tags such as SNAP-tag and CLIP-tag (both New England Biolabs).

BRD4: Bromodomain-containing protein 4 (BRD4) is a transcriptional regulator. As epigenetic “reader” protein BRD 4 is important for regulating fundamental processes in human health and diseases, including cancer development. As a member of the Bromodomains and Extraterminal (BET) family BRD4 is characterized by two tandem bromodomains (BD1. BD2) that recognize and bind acetylated lysine side chains and. Small molecule ligands (e.g. JQ-1) that bind both domains with comparable affinity have been developed. These compounds can be modified with linkers that can be further functionalized with TR-FRET donors and acceptors. We have used JQ-1 acid to synthesize tracer molecules functionalized with CoraFluors or TR-FRET acceptors.

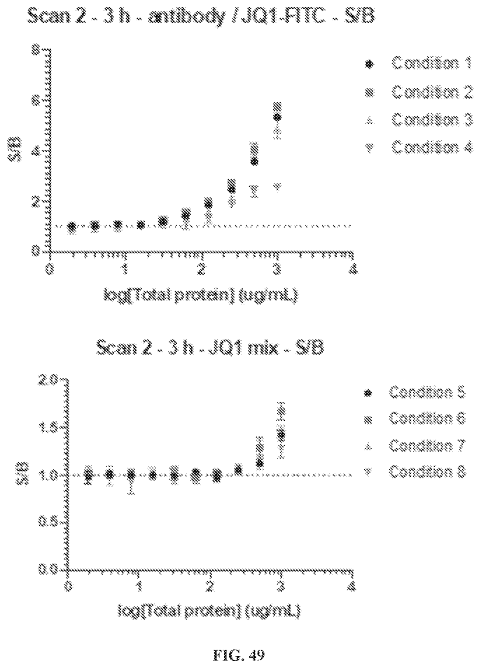

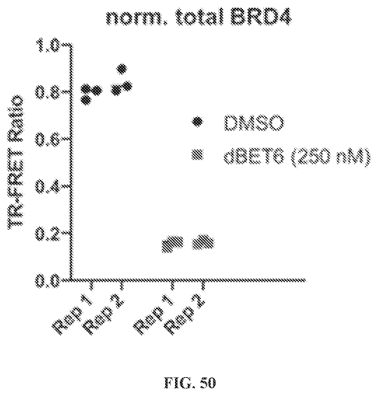

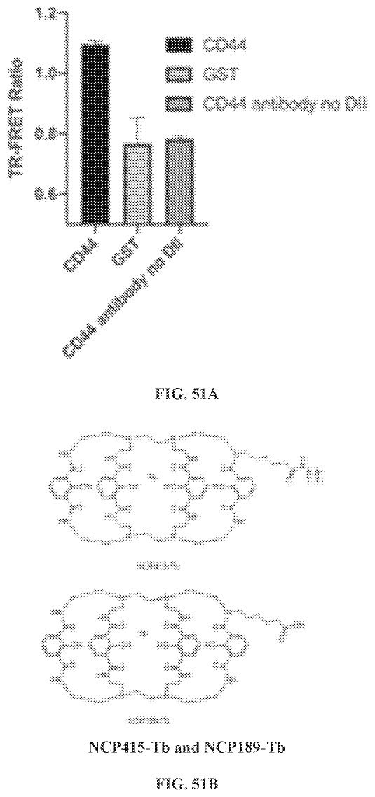

BRD4 is an attractive target in various oncological indications and traditional small molecule inhibitors are currently in clinical development. More recently targeted protein degradation approaches that result in the proteasomal elimination rather than simple inhibition of the POI are being developed (e.g., PROTACs). BRD4 is often used for proof-of-concept studies. This example shows by using a BRD4 specific IgG antibody in combination with TR-FRET donor-labeled anti-IgG nanobody and a TR-FRET acceptor labeled BRD4 inhibitor (JQ1) the applicability of this approach, which yields quantitative information directly from cell lysate (FIG. 49). Similar results are obtained for combination of donor-labeled JQ1 and acceptor-labeled JQ1.To demonstrate further proof-of-concept this example treated MCF7 cells with the reference compound dBET6, which is widely used in the literature to induce the degradation of BRD4. The results are shown in FIG. 50.

### Example 8—Tag-Free Approaches to Study Target Engagement, Protein-Protein Interaction or Quantify Protein Abundance

It is often desirable to probe ligand binding/displacement of a POI in cell lysates or live cells on native wildtype protein without the need to knock in an epitope tag or fusion-protein, or to ectopically express the POI as suitably tagged fusion protein. We here introduce novels approaches how this can be accomplished for target POI that a) features more than one small molecule ligand binding site for the same small molecule of for different small molecules, b) exist with two or more copies in multiprotein complexes (e.g. dimer, trimer, tetramer), c) POI as member of a multiprotein complex with at least one other protein that has a binding site for similar or different small molecule ligands, or d) are membrane bound proteins.

FRET-based proximity assays require both binding partners to be functionalized with the donor and acceptor, respectively. Because of the high costs and limited availability of the reagents, the TR-FRET donor is commonly directly linked to the target protein or indirectly through an antibody conjugate. However, our technology also allows us to readily functionalize small molecules and peptides, which enables these novel concepts that circumvent the need to tag the small molecule target. To demonstrate this, we provide the following distinct examples that illustrate the broad applicability. The experimental design is solely based on small molecule ligands. Since small molecules, unlike antibodies, can penetrate the plasma membrane, the approach will be compatible with detection and quantification of intracellular proteins in live cell, including native proteins without the need for epitope-tagging or expression as fusion proteins.

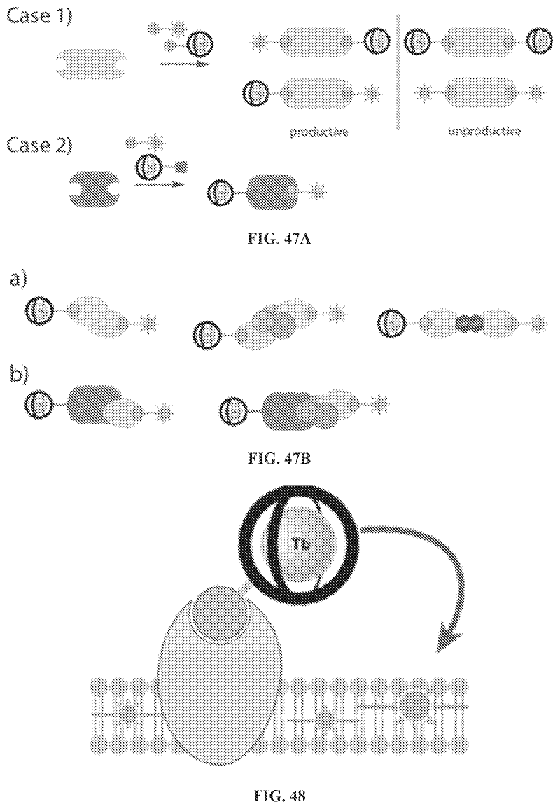

Studying POIs with more than one small molecule ligand binding site is schematically shown in FIG. 47A. For POI that feature more than one small molecule biding sites, two pairwise acceptor/donor-labeled small molecule tracers can be used. Referring to FIG. 47A: case 1) For POIs that can bind the same ligand at two or more sites (see below example BRD4) a mix of acceptor-labeled and donor-labeled ligand (preferably at equimolar concentration) will yield 50% productively labeled complexes at double occupancy (i.e. one site is occupied with a donor-labeled ligand and the second site is occupied with an acceptor-labeled ligand). Case 2) For POIs that can bind distinct small molecule ligands at two or more sites (see below example Keap1) one ligand type is acceptor-labeled and the other ligand type is donor-labeled. At double occupancy, this system will yield 100% productive complex.

Studying POIs with more than one copy in homo or hetero-complex is schematically shown in FIG. 47B, part a. A POI with at least one small molecule binding site that is member of a homocomplex, such as dimer, trimer, tetramer, or heterocomplexes with two or more copies of the same POI. Using the same approach as listed above (47a, Case 1) a TR-FRET system can be established by the combination of complementary labeled small molecule ligands (see example 6 related to Keap1).

Studying POI as member of a multiprotein complex with at least one other protein that has a binding site for similar or different small molecule ligands is schematically shown in FIG. 47B, part b.

### Example 9—studying membrane-bound proteins of interest

This example developed a novel TR-FRET assay approach to probe membrane proteins that have only a single ligand binding site. Instead of establishing a functional TR-FRET pair by utilizing two complementarily labeled ligands this example used environment sensitive dyes such as DiIC18(3) or DiI (“Dye Aye”) that function as TR-FRET acceptors. DiI, and the related DiO, DiD, DiR dyes, are lipophilic carbocyanine dyes that partition into cell membranes and are only brightly fluorescent when localized in membranes where they freely diffuse laterally. For long-lived TR-FRET donors such as CoraFluors it is important to appreciate that the acceptor is not required to always be within Förster distance to the donor, but it is sufficient if the acceptor is in proximity within the lifetime of the excited state of the donor. This effect, also known as diffusion quenching, is usually not desired in TR-FRET assays and becomes significant at micromolar acceptor concentration. This example utilizes it as an advantage as it allows the membrane to serve as efficient acceptor with the TR-FRET donor conjugated to a ligand that targets the membrane bound POI. This example includes two proof-of-concept studies to demonstrate feasibility of this approach (see CD44 antibody labeling and membrane targeted CoraFluor ligands). This approach is applicable to many pharmacologically relevant membrane proteins including GPCRs and RTKs.

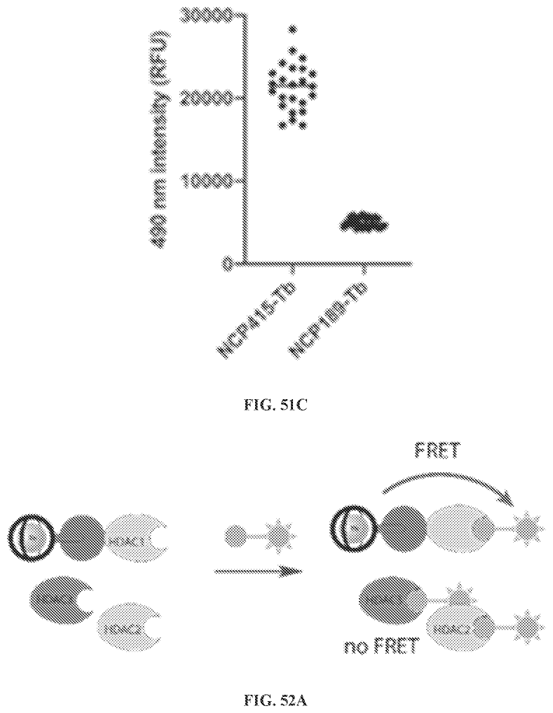

Membrane specific binding: To establish proof of concept for specific detection of membrane binding events by TR-FRET this example conducted two distinct experiments (FIG. 51). First, the example used a TR-FRET donor labeled anti-CD44 antibody, which recognizes a cell surface protein that is expressed on HEK293T cells. Cells incubated with anti-CD44 and DiI showed specific TR-FRET signal compared to cells incubated with anti-CD44 antibody alone or with DiI and a TR-FRET donor-labeled anti GST antibody.

Second, using MCF7 cells the example measured the TR-FRET signal obtained form DiI in combinations with a lipid tail modified CoraFluor (NCP145-Tb), which is also targeted to the plasma membrane, or an unmodified CoraFluor (NCP189-Tb), which is not targeted to the plasma membrane. Only NCP145-Tb resulted in strong specific signal.

### Example 10—Isoform and Complex Specific Assays

Utilizing small molecule probes to selectively target or profile certain POIs in cellular context can be impeded by the presence of homologs that bind exhibit comparable affinity for the small molecule ligand. This challenge is well appreciated for kinases. Similarly, it has been difficult to develop selective ligands for class I HDACs because of their highly conserved active site. Although medicinal chemistry programs have succeeded in developing selective inhibitors for of these targets, they have largely failed for others, including HDAC1 and HDAC2. Furthermore, HDACs1/2 are present in in distinct chromatin modifying complexes and the ability to directly assay the target engagement to one complex in the presence of other complexes would be highly desirable.

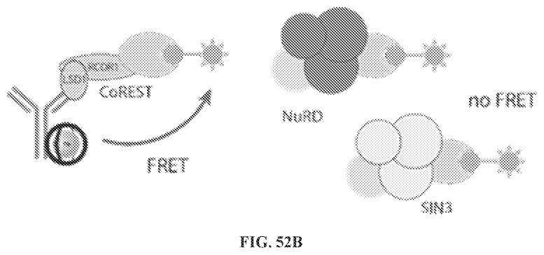

This example demonstrates the application of the technology for specific HDAC isoforms (FIG. 52a). In addition, the example shows it was possible to probe complex specific binding as illustrated for the CoREST complex (FIG. 52b).

### Numbered Paragraphs

In some embodiments, the present invention can be described with reference to the following numbered paragraphs.
Paragraph 1. A compound of Formula (1)

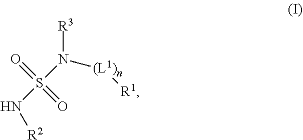

or a pharmaceutically acceptable salt thereof, wherein:
R³ is an ATP-binding moiety;
R² is an amino acid:
each L¹ is independently selected from O, S, S(═O)₂, NH, C═O, C═S, and C₁₋₆ alkylene;
n is an integer from 1 to 12; and
R¹ is a fluorophore.
Paragraph 2. The compound of paragraph 1, wherein the ATP-binding moiety is selected from any one of the following moieties:

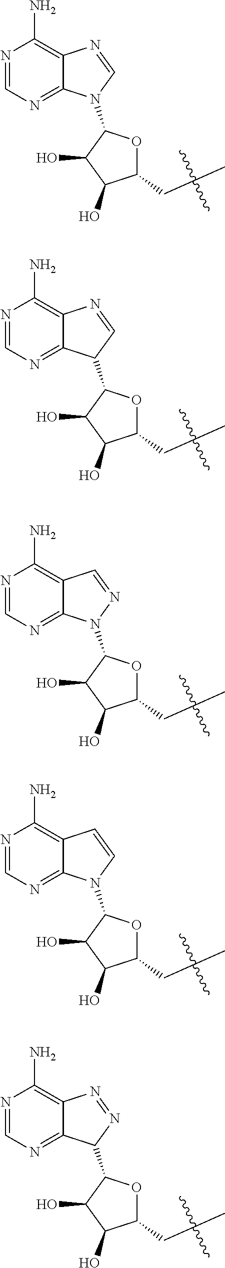 [SMILES: *C[C@H]1O[C@@H](n2cnc3c(N)ncnc32)[C@H](O)[C@@H]1O ; *C[C@H]1O[C@@H](C2C=Nc3c(N)ncnc32)[C@H](O)[C@@H]1O ; *C[C@H]1O[C@@H](n2ncc3c(N)ncnc32)[C@H](O)[C@@H]1O ; *C[C@H]1O[C@@H](n2ccc3c(N)ncnc32)[C@H](O)[C@@H]1O ; *C[C@H]1O[C@@H](C2N=Nc3c(N)ncnc32)[C@H](O)[C@@H]1O]

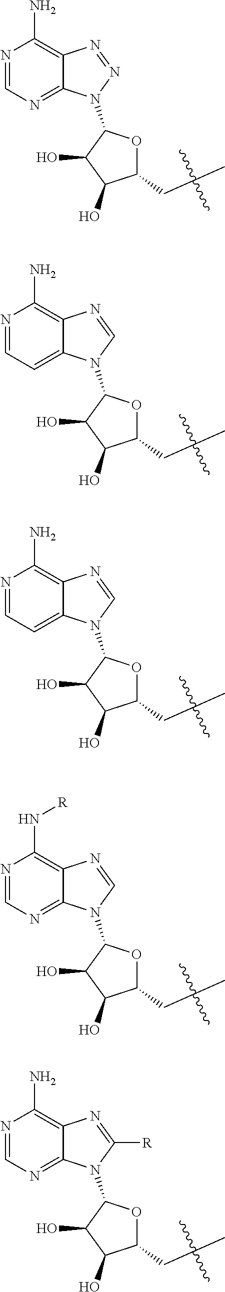

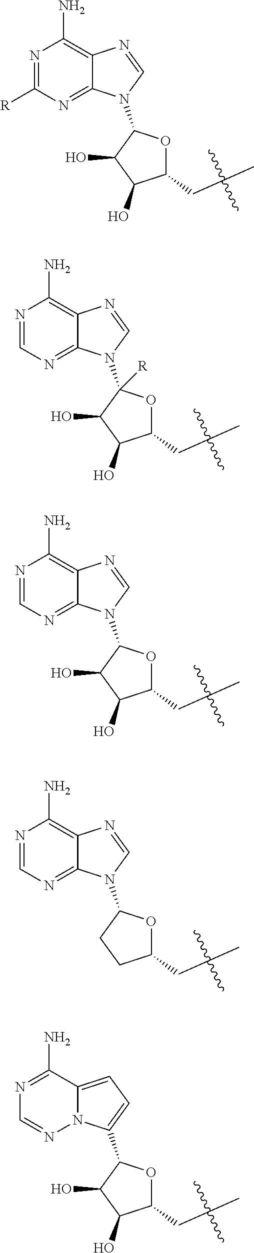

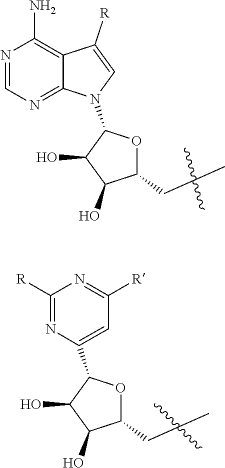

wherein Rand R are independently selected from H, C₁₋₃ alkyl, and C₁₋₃ haloalkyl.
Paragraph 3. The compound of paragraph 1, wherein the compound has Formula (I):

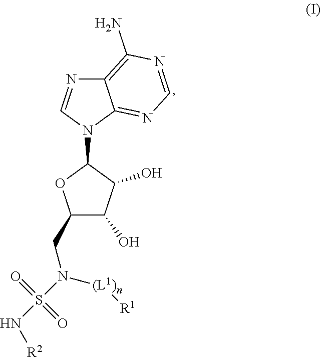

or a pharmaceutically acceptable salt thereof.
Paragraph 4. The compound of any one of paragraphs 1-3, where in the amino acid is selected from alanine. arginine, asparagine, aspartic acid, cysteine, glutamine, glutamic acid, glycine, histidine, isoleucine, leucine, isoleucine, lysine, methionine, phenylalanine, proline, serine, threonine, tryptophan, tyrosine, and valine.
Paragraph 5. The compound of paragraph 4, wherein the amino acid is proline.
Paragraph 6. The compound of paragraph 4, wherein the amino acid is phenylalanine.
Paragraph 7. The compound of paragraph 4, wherein the amino acid is isoleucine.
Paragraph 8. The compound of paragraph 4, wherein the amino acid is glutamic acid.
Paragraph 9. The compound of any one of paragraph 1-8, wherein each L¹ is independently selected from O, NH, C═O, C═S, and C₁₋₆ alkylene.
Paragraph 10. The compound of any one of paragraphs 1-8, wherein the moiety (L¹)ₙ comprises (C═O)O.
Paragraph 11. The compound of any one of paragraphs 1-8, wherein the moiety (L¹)ₙ comprises NH(C═O)O.
Paragraph 12. The compound of any one of paragraphs 1-8, wherein the moiety (L¹)ₙ comprises (C═O)—C₁₋₆ alkylene.
Paragraph 13. The compound of any one of paragraphs 1-8, wherein the moiety (L¹)ₙ comprises NH(C═S)NH.
Paragraph 14. The compound of any one of paragraphs 1-8, wherein the moiety (L¹) comprises NH(C═O).
Paragraph 15. The compound of any one of paragraphs 1-8, wherein the moiety (L¹)ₙ comprises NH(C═O)NH.
Paragraph 16. The compound of any one of paragraphs 1-8, wherein the moiety (L¹)ₙ comprises OCH₂CH₂O.
Paragraph 17. The compound of any one of paragraphs 1-8, wherein the moiety (L) comprises OCH₂CH₂NH.
Paragraph 18. The compound of paragraph 1, wherein the compound has formula:

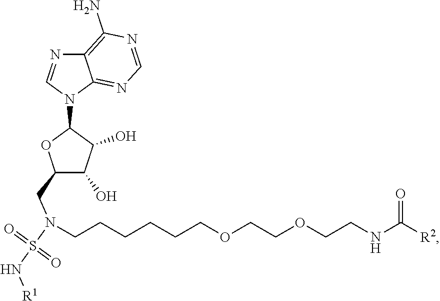

or a pharmaceutically acceptable salt thereof.
Paragraph 19. The compound of any one of paragraphs 1-18, wherein R¹ is a FRET acceptor fluorophore.
Paragraph 20. The compound of paragraph 19, wherein R¹ is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light). Cy3 yellow, TRITC yellow, Alexa fluor 546 yellow, Alexa fluor 555 3 yellow, R-phycoerythrin (PE) 480; yellow, Rhodamine Red-X orange. Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.
Paragraph 21. The compound of any one of paragraphs 1-17, having formula:

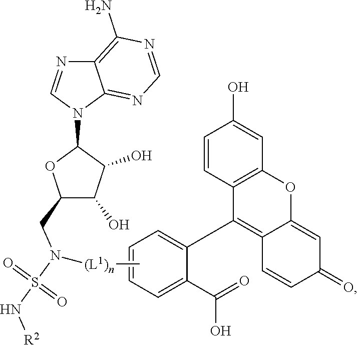

or a pharmaceutically acceptable salt thereof.
Paragraph 22. The compound of paragraph 1, selected from any one of the following compounds:

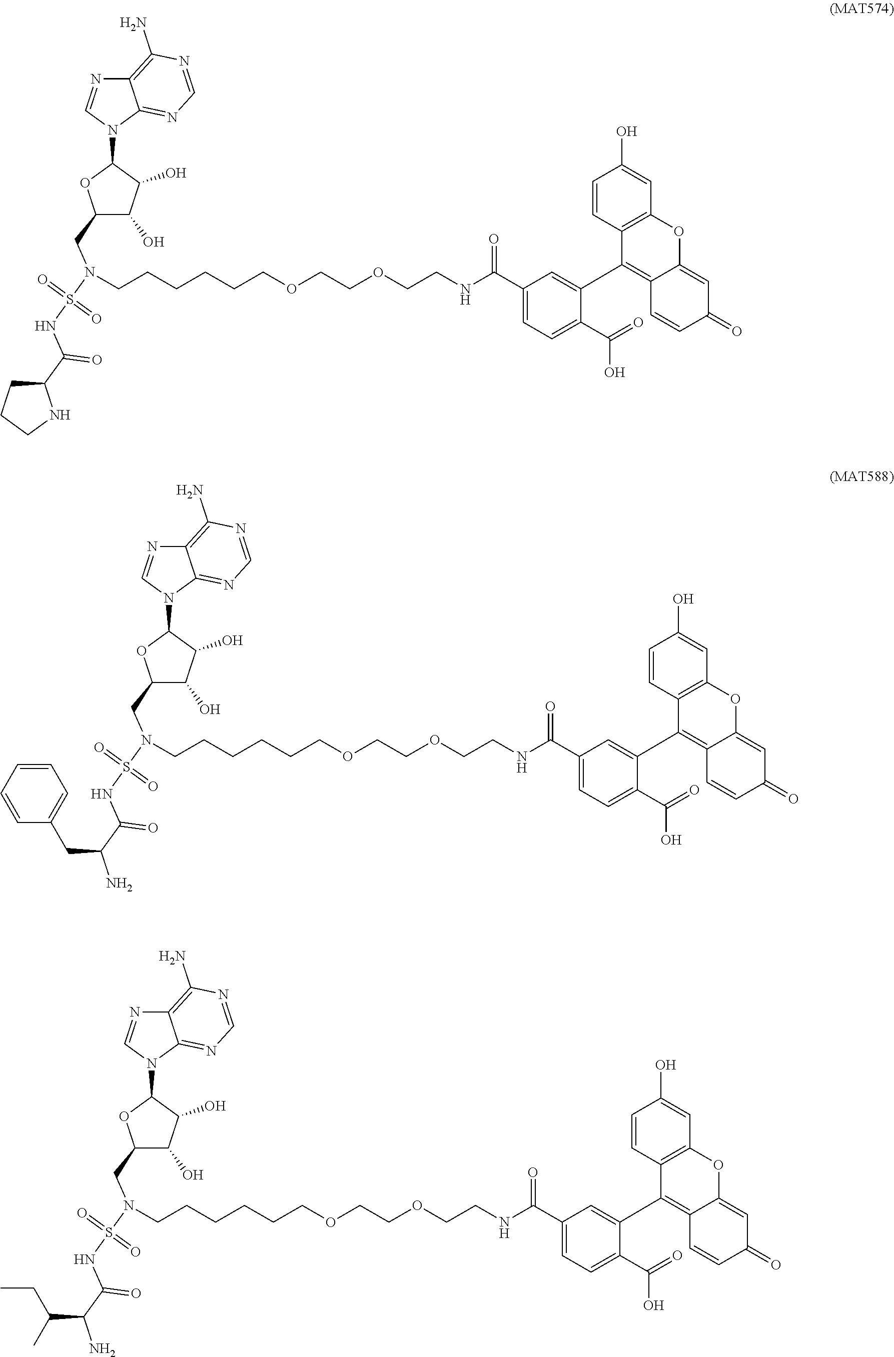 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H]2CCCN2)[C@@H](O)[C@H]1O ; Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H](N)Cc2ccccc2)[C@@H](O)[C@H]1O ; CCC(C)[C@H](N)C(=O)NS(=O)(=O)N(CCCCCCOCCOCCNC(=O)c1ccc(C(=O)O)c(-c2c3ccc(=O)cc-3oc3cc(O)ccc23)c1)C[C@H]1O[C@@H](n2cnc3c(N)ncnc32)[C@H](O)[C@@H]1O]

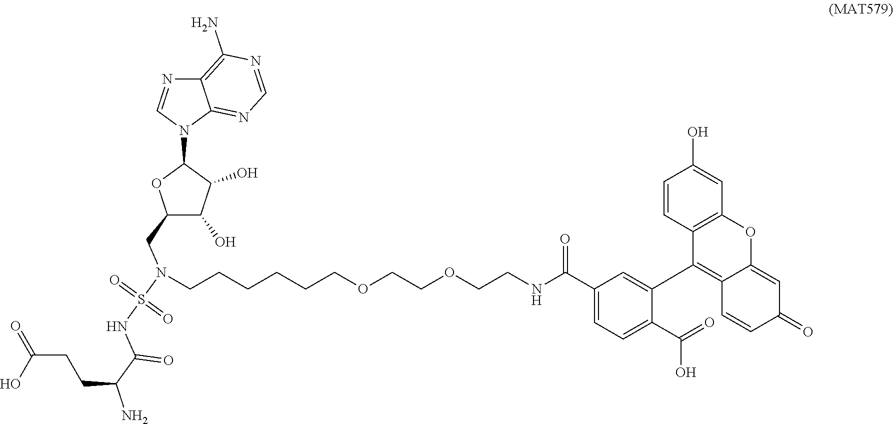 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H](N)CCC(=O)O)[C@@H](O)[C@H]1O]

or a pharmaceutically acceptable salt thereof.
Paragraph 23. A compound of Formula (II):

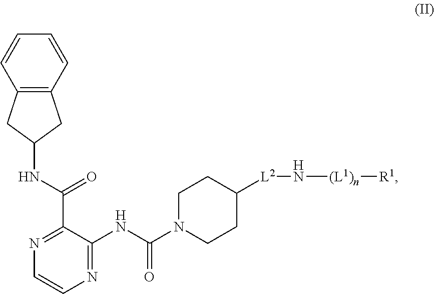

or a pharmaceutically acceptable salt thereof, wherein:
L² is C₁₋₃ alkylene; or L² is absent;
each L¹ is independently selected from O, S, S(═O)₂, NH, C═O, C═S, and C₁₋₆ alkylene;
n is an integer from 1 to 12. and
R¹ is a fluorophore.
Paragraph 24. The compound of paragraph 23, wherein the compound has formula:

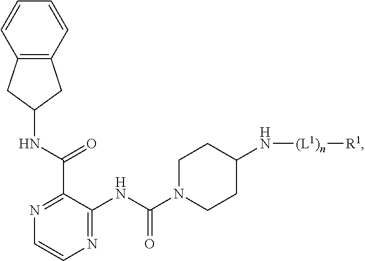

or a pharmaceutically acceptable salt thereof.
Paragraph 25. The compound of paragraph 23. wherein the compound has formula:

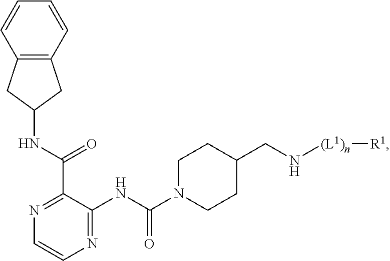

or a pharmaceutically acceptable salt thereof.
Paragraph 26. The compound of any one of paragraphs 23-25, wherein each L¹ is independently selected from O, NH, C═O, C═S, and C₁₋₆ alkylene.
Paragraph 27. The compound of any one of paragraphs 23-25, wherein the moiety (L¹) comprises (C═O)O.
Paragraph 28. The compound of any one of paragraphs 23-25, wherein the moiety (L¹)ₙ comprises NH(C═O)O.
Paragraph 29. The compound of any one of paragraphs 23-25, wherein the moiety (L¹)ₙ comprises (C═O)—C₁₋₆ alkylene.
Paragraph 30. The compound of any one of paragraphs 23-25, wherein the moiety (L) comprises NH(C═S)NH.
Paragraph 31. The compound of any one of paragraphs 23-25, wherein the moiety (L¹)ₙ comprises NH(C═O).
Paragraph 32. The compound of any one of paragraphs 23-25, wherein the moiety (L¹)ₙ comprises NH(C═O)NH.
Paragraph 33. The compound of any one of paragraphs 23-25, wherein the moiety (L¹)ₙ comprises OCH₂CH₂O.
Paragraph 34. The compound of any one of paragraphs 23-25, wherein the moiety (L¹) comprises OCH₂CH₂NH.
Paragraph 35. The compound of any one of paragraph 23-25, wherein the moiety (L¹)ₙ comprises any one of the following fragments:

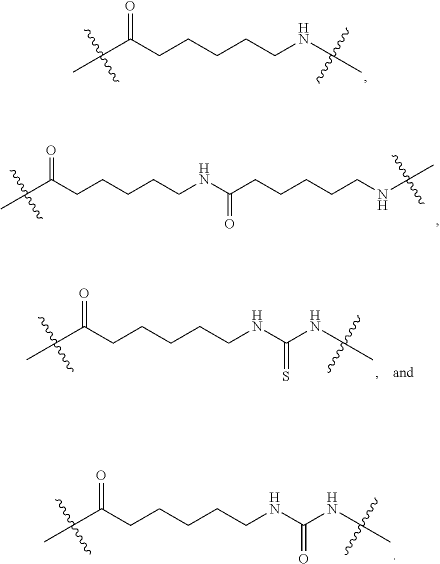 [SMILES: *NCCCCCC(*)=O ; *NCCCCCC(=O)NCCCCCC(*)=O ; *NC(=S)NCCCCCC(*)=O ; *NC(=O)NCCCCCC(*)=O]

Paragraph 36. The compound of any one of paragraph 23-35, wherein R¹ is a FRET acceptor fluorophore.
Paragraph 37. The compound of paragraph 36. wherein R¹ is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red. Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.
Paragraph 38. The compound of paragraph 23, having formula:

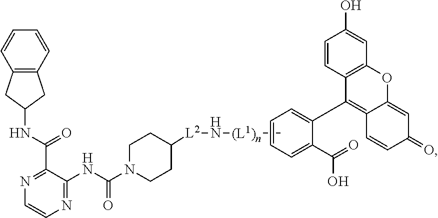

or a pharmaceutically acceptable salt thereof.
Paragraph 39. The compound of paragraph 23, wherein the compound of Formula (II) is selected from any one of the following compounds;

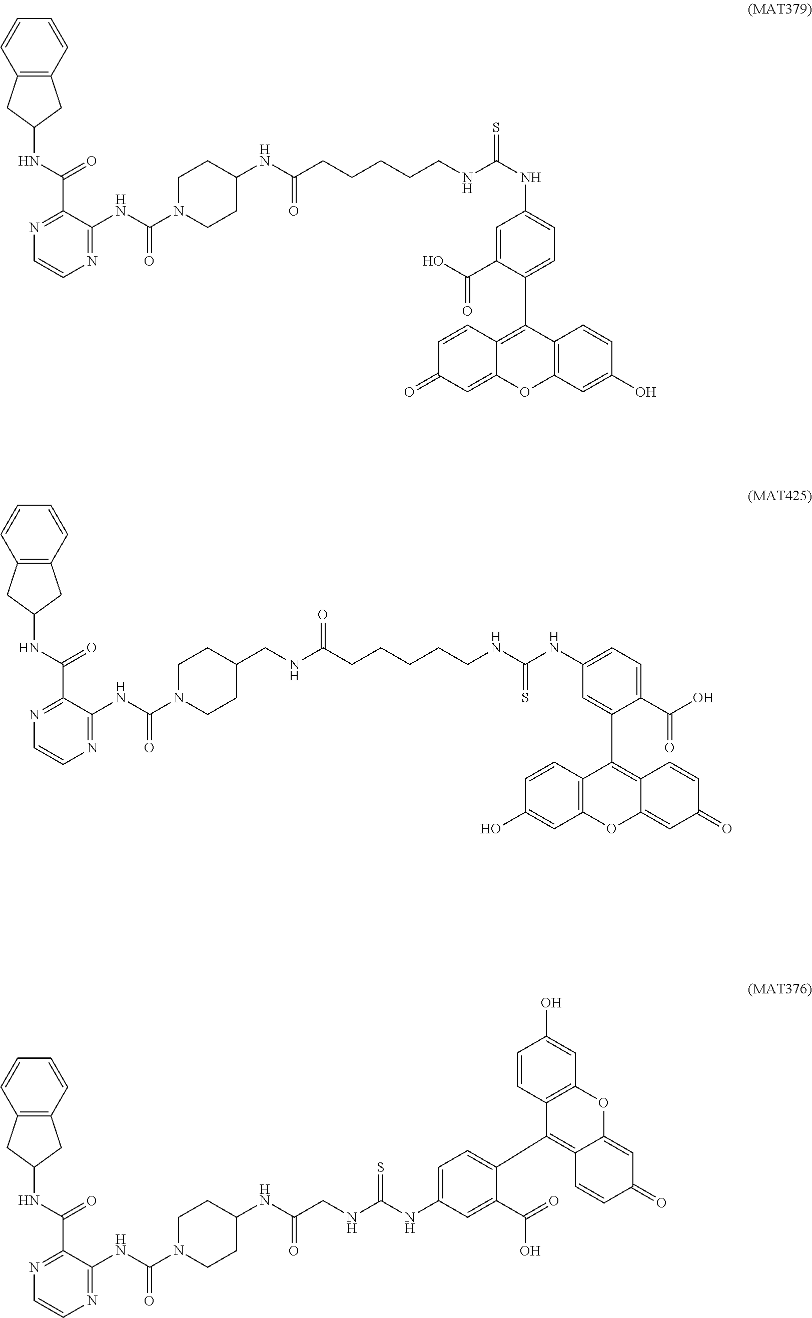 [SMILES: O=C(CCCCCNC(=S)Nc1ccc(-c2c3ccc(=O)cc-3oc3cc(O)ccc23)c(C(=O)O)c1)NC1CCN(C(=O)Nc2nccnc2C(=O)NC2Cc3ccccc3C2)CC1 ; O=C(CCCCCNC(=S)Nc1ccc(C(=O)O)c(-c2c3ccc(=O)cc-3oc3cc(O)ccc23)c1)NCC1CCN(C(=O)Nc2nccnc2C(=O)NC2Cc3ccccc3C2)CC1 ; O=C(CNC(=S)Nc1ccc(-c2c3ccc(=O)cc-3oc3cc(O)ccc23)c(C(=O)O)c1)NC1CCN(C(=O)Nc2nccnc2C(=O)NC2Cc3ccccc3C2)CC1]

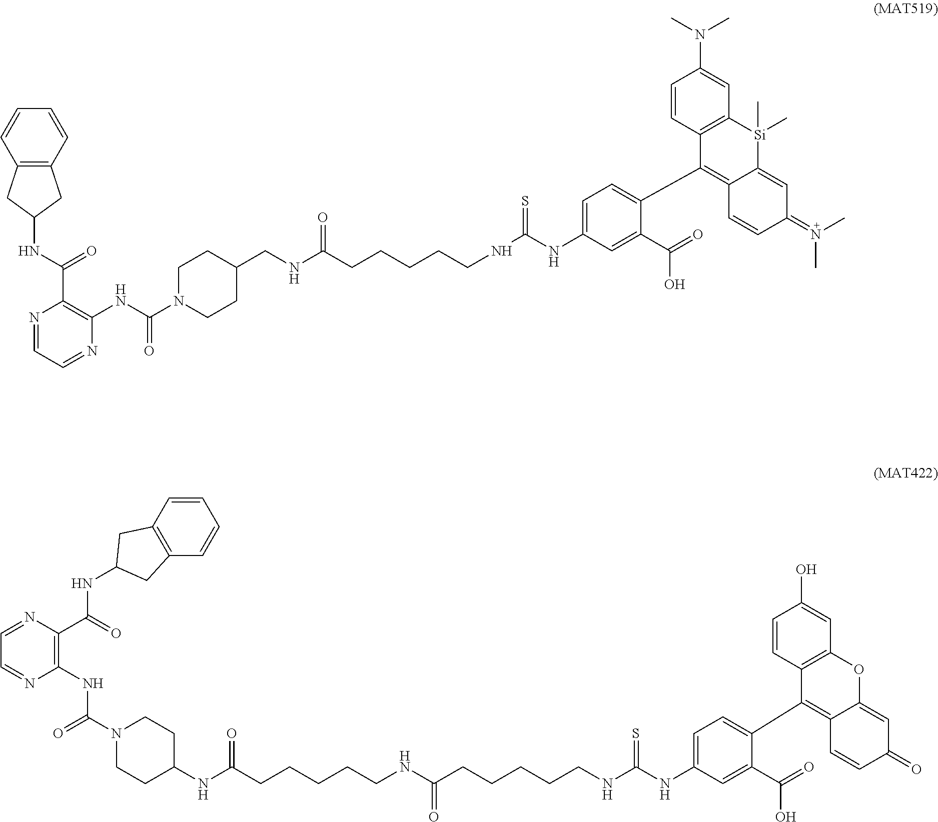

or a pharmaceutically acceptable salt thereof.
Paragraph 40. A compound of Formula (III):

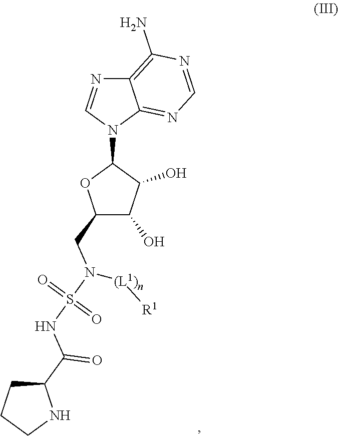

or a pharmaceutically acceptable salt thereof, wherein:
each L¹ is independently selected from O, S, S(═O)₂, NH, C═O, C═S, and C₁₋₆ alkylene;
n is an integer from 1 to 12; and
R¹ is a fluorophore.
Paragraph 41. The compound of paragraph 40, wherein each L¹ is independently selected from O, NH, C═O, C═S, and C₁₋₆ alkylene.
Paragraph 42. The compound of paragraph 40, wherein the moiety (L¹)ₙ comprises (C═O)O.
Paragraph 43. The compound of paragraph 40, wherein the moiety (L¹)ₙ comprises NH(C═O)O.
Paragraph 44. The compound of paragraph 40, wherein the moiety (L¹)ₙ comprises (C═O)—C₁₋₆ alkylene.
Paragraph 45. The compound of paragraph 40, wherein the moiety (L¹)ₙ comprises NH(C═S)NH.
Paragraph 46. The compound of paragraph 40, wherein the moiety (L¹)ₙ comprises NH(C═O).
Paragraph 47. The compound of paragraph 40, wherein the moiety (L¹)ₙ comprises NH(C═O)NH.
Paragraph 48. The compound of paragraph 40, wherein the moiety (L¹)ₙ comprises OCH₂CH₂O.
Paragraph 49. The compound of paragraph 40, wherein the moiety (L¹)ₙ comprises OCH₂CH₂NH.
Paragraph 50. The compound of any one of paragraphs 40-49, wherein R¹ is selected from fluorescein, AF488, hydroxycoumarin blue, methoxycoumarin blue, alexa fluor blue, aminocoumarin blue. Cy2 green (dark), FAM green (dark), alexa fluor 488 green (light), fluorescein FITC green (light), alexa fluor 430 green (light). Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.
Paragraph 51. The compound of paragraph 40. having formula:

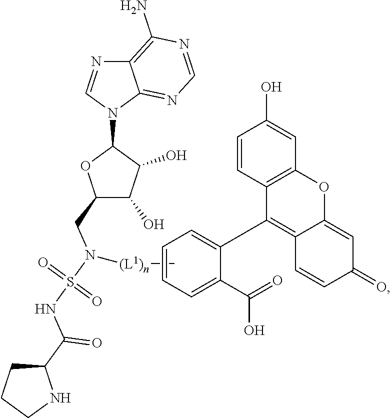

or a pharmaceutically acceptable salt thereof.
Paragraph 52. The compound of paragraph 40, having formula:

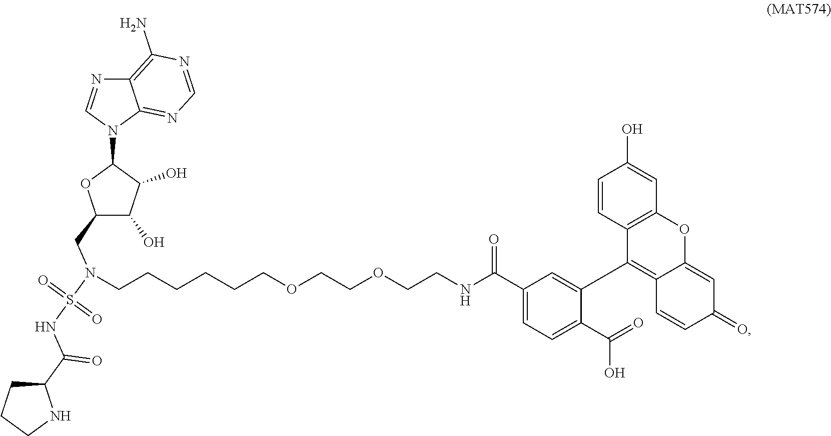 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H]2CCCN2)[C@@H](O)[C@H]1O]

or a pharmaceutically acceptable salt thereof.
Paragraph 53. A compound of Formula (IV):

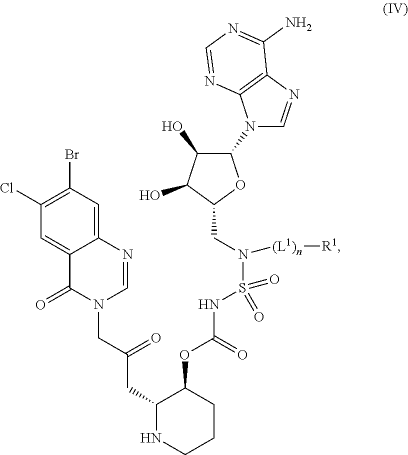

or a pharmaceutically acceptable salt thereof, wherein:
each L¹ is independently selected from O, S, S(═O)₂, NH, C═O, C═S, and C₁₋₆ alkylene;
n is an integer from 1 to 12; and
R¹ is a fluorophore.
Paragraph 54. The compound of paragraph 53, wherein each L¹ is independently selected from O, NH, C═O, C═S, and C₁₋₆ alkylene.
Paragraph 55. The compound of paragraph 53, wherein the moiety (L¹)ₙ comprises (C═O)O.
Paragraph 56. The compound of paragraph 53, wherein the moiety (L¹)ₙ comprises NH(C═O)O.
Paragraph 57. The compound of paragraph 53, wherein the moiety (L¹)ₙ comprises (C═O)—C₁₋₆ alkylene.
Paragraph 58. The compound of paragraph 53, wherein the moiety (L¹)ₙ comprises NH(C═S)NH.
Paragraph 59. The compound of paragraph 53, wherein the moiety (L¹)ₙ comprises NH(C═O).
Paragraph 60. The compound of paragraph 53, wherein the moiety (L¹)ₙ comprises NH(C═O)NH.
Paragraph 61. The compound of paragraph 53, wherein the moiety (L¹)ₙ comprises OCH₂CH₂O.
Paragraph 62. The compound of paragraph 53, wherein the moiety (L¹)ₙ comprises OCH₂CH₂NH.
Paragraph 63. The compound of paragraph 53, wherein the compound has formula:

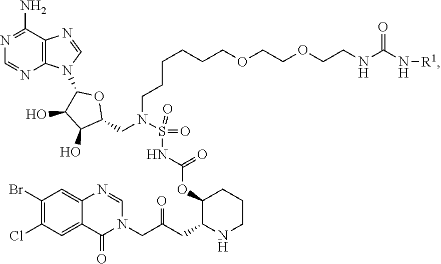

or a pharmaceutically acceptable salt thereof.
Paragraph 64. The compound of any one of paragraphs 53-63, wherein R¹ is a FRET acceptor fluorophore.
Paragraph 65. The compound of paragraph 64, wherein R¹ is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light). Cy3 yellow, TRITC yellow, Alexa fluor 546 yellow, Alexa fluor 555 3 yellow, R-phycoerythrin (PE) 480; yellow, Rhodamine Red-X orange. Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.
Paragraph 66. The compound of paragraph 53, wherein the compound has formula:

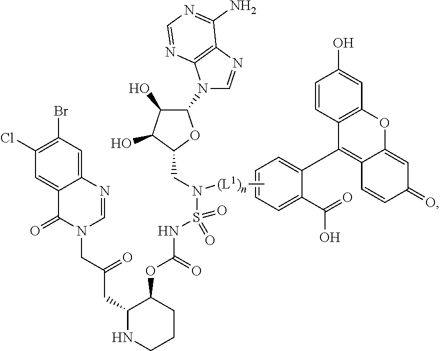

or a pharmaceutically acceptable salt thereof.
Paragraph 67. The compound of paragraph 53. having formula:

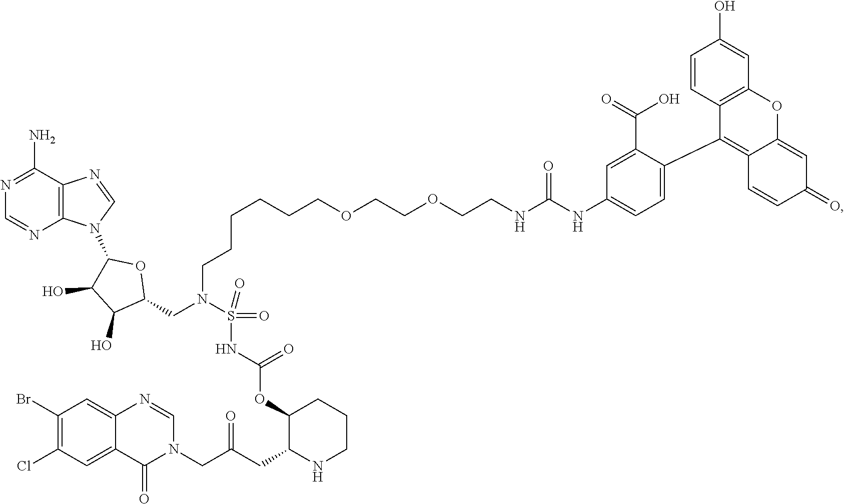 [SMILES: Nc1ncnc2c1ncn2[C@@H]1O[C@H](CN(CCCCCCOCCOCCNC(=O)Nc2ccc(-c3c4ccc(=O)cc-4oc4cc(O)ccc34)c(C(=O)O)c2)S(=O)(=O)NC(=O)O[C@H]2CCCN[C@@H]2CC(=O)Cn2cnc3cc(Br)c(Cl)cc3c2=O)[C@@H](O)[C@H]1O]

or a pharmaceutically acceptable salt thereof.
Paragraph 68. A compound of Formula (V):

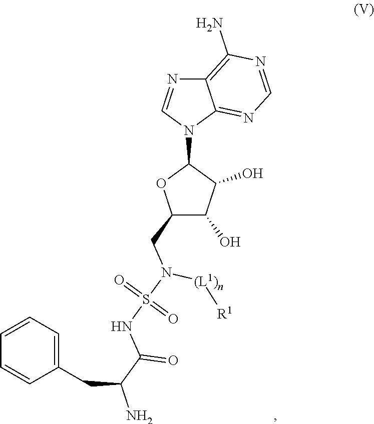

or a pharmaceutically acceptable salt thereof, wherein:
each L¹ is independently selected from O, S, S(═O)₂, NH, C═O, C═S, and C₁₋₆ alkylene;
n is an integer from 1 to 12; and
R¹ is a fluorophore.
Paragraph 69. The compound of paragraph 68, wherein each L¹ is independently selected from O, NH, C═O, C═S, and C₁₋₆ alkylene.
Paragraph 70. The compound of paragraph 68, wherein the moiety (L¹)ₙ comprises (C═O)O.
Paragraph 71. The compound of paragraph 68, wherein the moiety (L¹)ₙ comprises NH(C═O)O.
Paragraph 72. The compound of paragraph 68, wherein the moiety (L¹)ₙ comprises (C═O)—C₁₋₆ alkylene.
Paragraph 73. The compound of paragraph 68, wherein the moiety (L¹)ₙ comprises NH(C═S)NH.
Paragraph 74. The compound of paragraph 68, wherein the moiety (L¹)ₙ comprises NH(C═O).
Paragraph 75. The compound of paragraph 68, wherein the moiety (L¹)ₙ comprises NH(C═O)NH.
Paragraph 76. The compound of paragraph 68, wherein the moiety (L¹)ₙ comprises OCH₂CH₂O.
Paragraph 77. The compound of paragraph 68, wherein the moiety (L¹)ₙ comprises OCH₂CH₂NH.
Paragraph 78. The compound of paragraph 68, wherein the compound has formula:

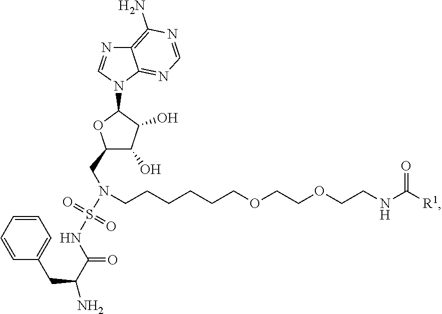

or a pharmaceutically acceptable salt thereof.
Paragraph 79. The compound of any one of paragraphs 68-78, wherein R¹ is a FRET acceptor fluorophore.
Paragraph 80. The compound of paragraph 79. wherein R¹ is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light). Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red. Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.
Paragraph 81. The compound of paragraph 68, having formula:

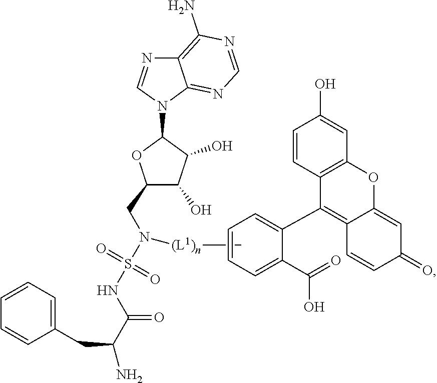

or a pharmaceutically acceptable salt thereof.
Paragraph 82. The compound of paragraph 68, having formula:

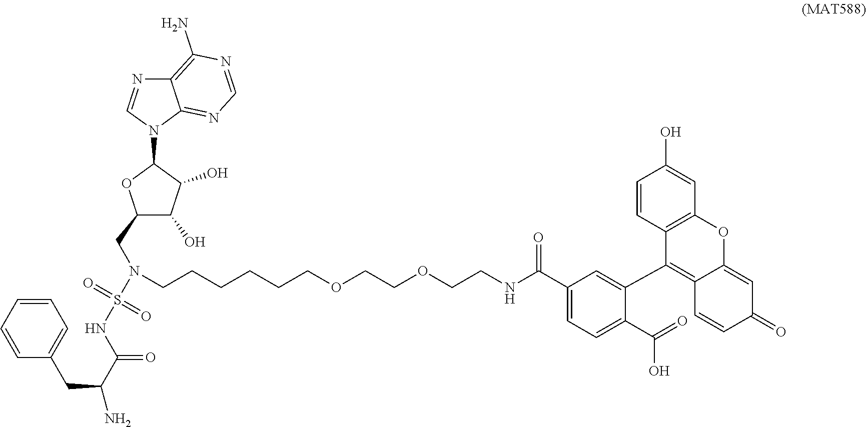 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H](N)Cc2ccccc2)[C@@H](O)[C@H]1O]

or a pharmaceutically acceptable salt thereof.
Paragraph 83. A compound of Formula (VI):

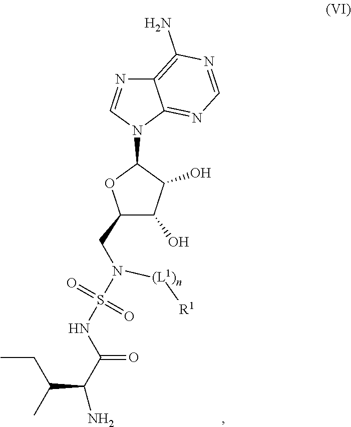

or a pharmaceutically acceptable salt thereof, wherein:
each L¹ is independently selected from O, S, S(═O)₂, NH, C═O, C═S, and C₁₋₆ alkylene;
n is an integer from 1 to 12; and
R¹ is a fluorophore.
Paragraph 84. The compound of paragraph 83, wherein each L¹ is independently selected from O, NH, C═O, C═S, and C₁₋₆ alkylene.
Paragraph 85. The compound of paragraph 83, wherein the moiety (L¹)ₙ comprises (C═O)O.
Paragraph 86. The compound of paragraph 83, wherein the moiety (L¹)ₙ comprises NH(C═O)O.
Paragraph 87. The compound of paragraph 83, wherein the moiety (L¹)ₙ comprises (C═O)—C₁₋₄ alkylene.
Paragraph 88. The compound of paragraph 83, wherein the moiety (L¹)ₙ comprises NH(C═S)NH.
Paragraph 89. The compound of paragraph 83, wherein the moiety (L¹)ₙ comprises NH(C═O).
Paragraph 90. The compound of paragraph 81, wherein the moiety (L¹)ₙ comprises NH(C═O)NH.
Paragraph 91. The compound of paragraph 83, wherein the moiety (L¹)ₙ comprises OCH₂CH₂O.
Paragraph 92. The compound of paragraph 83, wherein the moiety (L¹)ₙ comprises OCH₂CH₂NH.
Paragraph 93. The compound of paragraph 83, wherein the compound has formula:

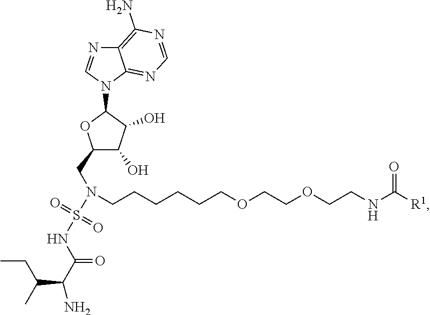

or a pharmaceutically acceptable salt thereof.
Paragraph 94. The compound of any one of paragraphs 83-93, wherein R¹ is a FRET acceptor fluorophore.
Paragraph 95. The compound of paragraph 94. wherein R¹ is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red. Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.
Paragraph 96. The compound of paragraph 83, having formula:

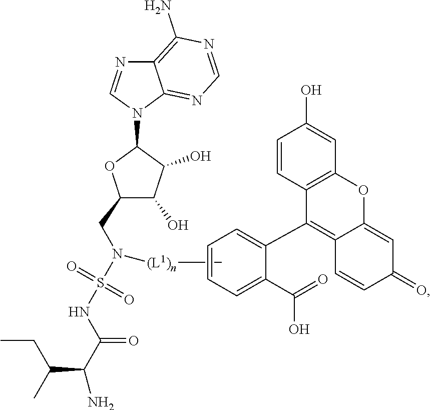

or a pharmaceutically acceptable salt thereof.
Paragraph 97. The compound of paragraph 83, having formula:

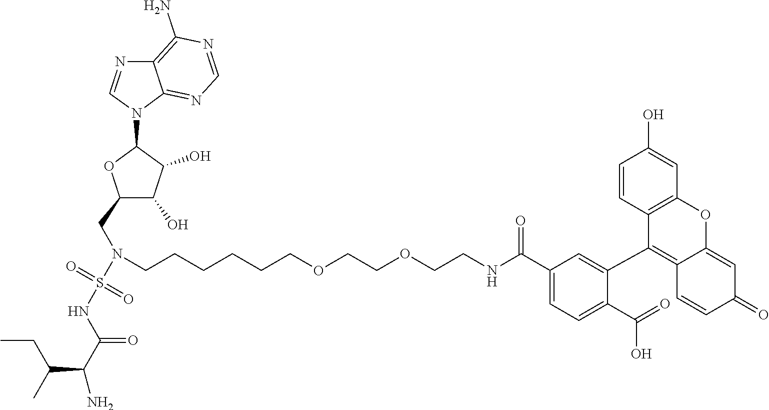 [SMILES: CCC(C)[C@H](N)C(=O)NS(=O)(=O)N(CCCCCCOCCOCCNC(=O)c1ccc(C(=O)O)c(-c2c3ccc(=O)cc-3oc3cc(O)ccc23)c1)C[C@H]1O[C@@H](n2cnc3c(N)ncnc32)[C@H](O)[C@@H]1O]

or a pharmaceutically acceptable salt thereof.
Paragraph 98. A compound of Formula (VII):

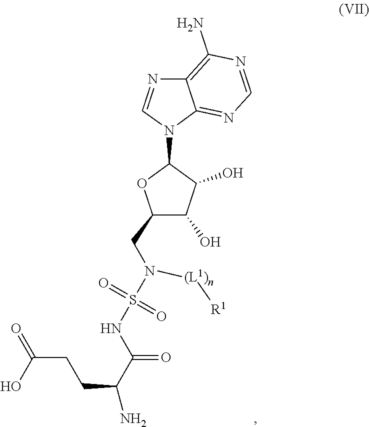

or a pharmaceutically acceptable salt thereof, wherein:
each L¹ is independently selected from O, S, S(═O)₂, NH, C═O, C═S, and C₁₋₆ alkylene;
n is an integer from 1 to 12; and
R¹ is a fluorophore.
Paragraph 99. The compound of paragraph 98, wherein each L¹ is independently selected from O, NH, C═O, C═S, and C₁₋₆ alkylene.
Paragraph 100. The compound of paragraph 98, wherein the moiety (L¹)ₙ comprises (C═O)O.
Paragraph 101. The compound of paragraph 98, wherein the moiety (L¹)ₙ comprises NH(C═O)O.
Paragraph 102. The compound of paragraph 98, wherein the moiety (L¹)ₙ comprises (C═O)—C₁₋₄ alkylene.
Paragraph 103. The compound of paragraph 98, wherein the moiety (L¹)ₙ comprises NH(C═S)NH.
Paragraph 104. The compound of paragraph 96, wherein the moiety (L¹)ₙ comprises NH(C═O).
Paragraph 105. The compound of paragraph 98, wherein the moiety (L¹)ₙ comprises NH(C═O)NH.
Paragraph 106. The compound of paragraph 98, wherein the moiety (L¹)ₙ comprises OCH₂CH₂O.
Paragraph 107. The compound of paragraph 98, wherein the moiety (L¹)ₙ comprises OCH₂CH₂NH.
Paragraph 108. The compound of paragraph 98, having formula:

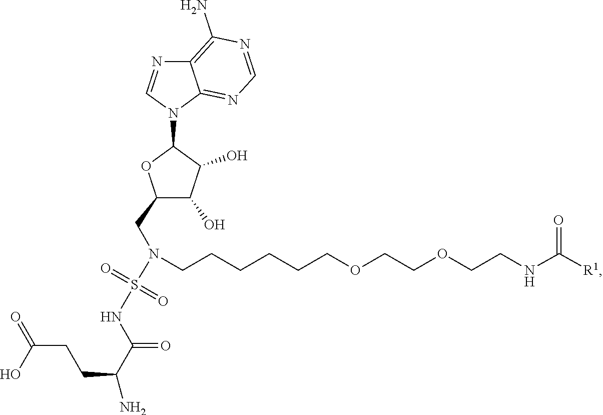

or a pharmaceutically acceptable salt thereof.
Paragraph 109. The compound of any one of paragraphs 98-108, wherein R¹ is a FRET acceptor fluorophore.
Paragraph 110. The compound of paragraph 109, wherein R¹ is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow. Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.
Paragraph 111. The compound of paragraph 98, having formula:

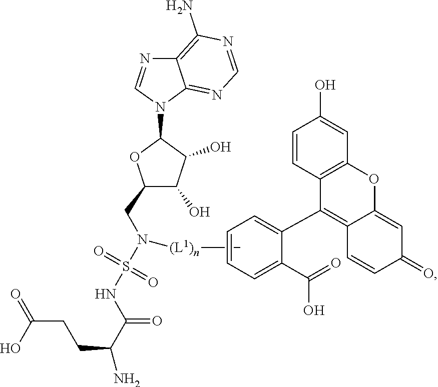

or a pharmaceutically acceptable salt thereof.
Paragraph 112. The compound of paragraph 98, having formula:

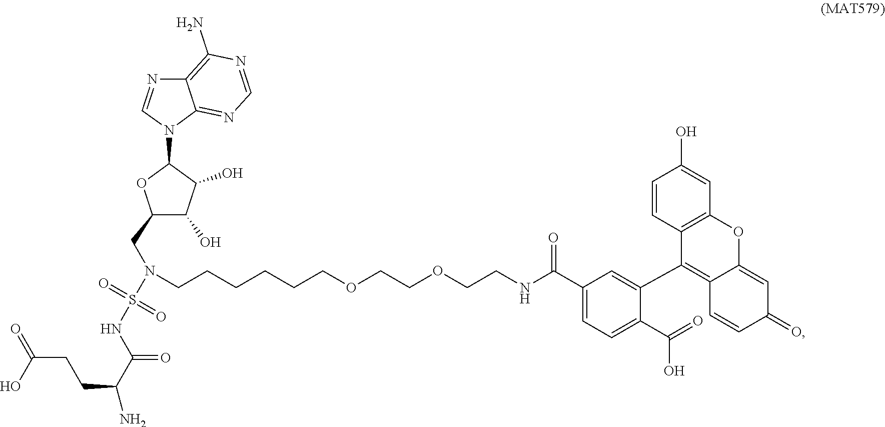 [SMILES: Nc1ncnc2c1ncn2[C@@H]1O[C@H](CN(CCCCCCOCCOCCNC(=O)c2ccc(C(=O)O)c(-c3c4ccc(=O)cc-4oc4cc(O)ccc34)c2)S(=O)(=O)NC(=O)[C@@H](N)CCC(=O)O)[C@@H](O)[C@H]1O]

or a pharmaceutically acceptable salt thereof.
Paragraph 113. A compound of Formula (A):

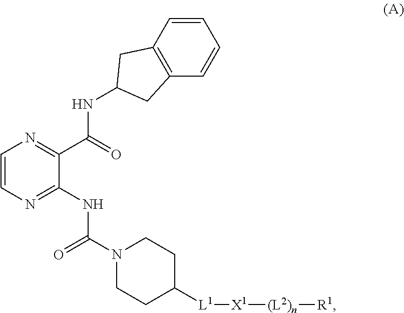

or a pharmaceutically acceptable salt thereof, wherein:
L¹ is C₁₋₃ alkylene, or L¹ is absent:
X¹ is selected from O and NR^{N};
R^{N} is selected from H, C₁₋₃ alkyl, and C₁₋₃ haloalkyl;
   each L² is independently selected from O, S, S(═O)₂, NR^{N}, C═O, C═S, and C₁₋₆ alkylene;
n is an integer from 0 to 12; and
R¹ is selected from H, C₁₋₆ alkyl, and a protecting group.
Paragraph 114. The compound of paragraph 113, wherein L¹ is absent.
Paragraph 115. The compound of paragraph 113, wherein L¹ is C₁₋₃ alkylene.
Paragraph 116. The compound of paragraph 115, wherein L¹ is selected from methylene, 1,2-ethylene, 1,1-ethylene, and propylene.
Paragraph 117. The compound of paragraph 115, wherein L¹ is methylene.
Paragraph 118. The compound of any one of paragraphs 113-117, wherein X¹ is O.
Paragraph 119. The compound of any one of paragraphs 113-117, wherein X¹ is NH.
Paragraph 120. The compound of paragraph 113, wherein the compound of Formula (A) has formula:

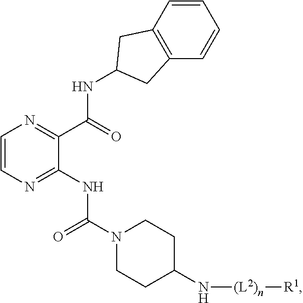

or a pharmaceutically acceptable salt thereof.
Paragraph 121. The compound of paragraph 113, wherein the compound of Formula (A) has formula:

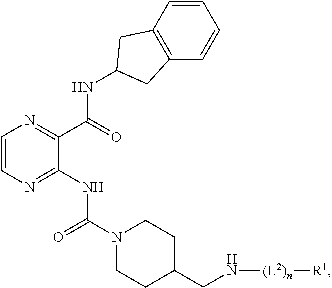

or a pharmaceutically acceptable salt thereof.
Paragraph 122. The compound of any one of paragraphs 113-121, wherein n is 0.
Paragraph 123. The compound of any one of paragraphs 113-121, wherein n is an integer from 1 to 12.
Paragraph 124. The compound of paragraph 123, wherein each L² is independently selected from O, S, NH, C═O, C═S, and C₁₋₆ alkylene.
Paragraph 125. The compound of paragraph 124, wherein the moiety (L¹)ₙ comprises (C═O)O.
Paragraph 126. The compound of paragraph 124, wherein the moiety (L¹)ₙ comprises NH(C═O)O.
Paragraph 127. The compound of paragraph 124, wherein the moiety (L¹)ₙ comprises (C═O)—C₁₋₆ alkylene.
Paragraph 128. The compound of paragraph 124, wherein the moiety (L¹)ₙ comprises NH(C═O).
Paragraph 129. The compound of paragraph 124, wherein the moiety (L²)ₙ comprises NH(C═O)NH.
Paragraph 130. The compound of paragraph 124, wherein the moiety (L²)ₙ comprises NH(C═S)NH.
Paragraph 131. The compound of paragraph 124, wherein the moiety (L²)ₙ comprises any one of the following fragments:

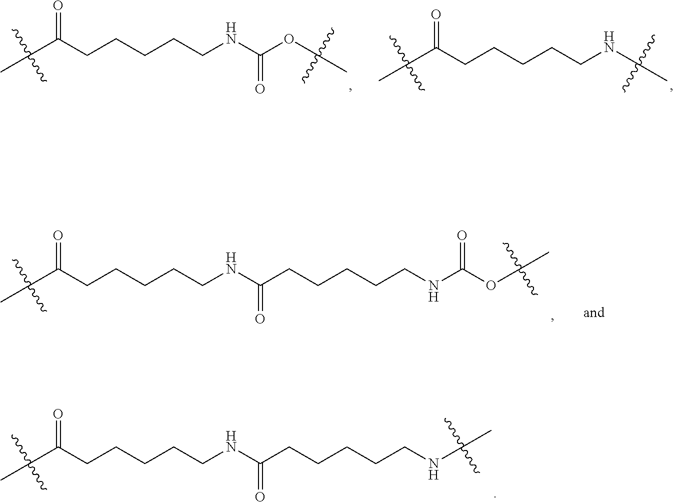 [SMILES: *OC(=O)NCCCCCC(*)=O ; *NCCCCCC(*)=O ; *OC(=O)NCCCCCC(=O)NCCCCCC(*)=O ; *NCCCCCC(=O)NCCCCCC(*)=O]

Paragraph 132. The compound of any one of paragraphs 113-131, wherein R¹ is H.
Paragraph 133. The compound of any one of paragraphs 113-131, wherein R¹ is C₁₋₆ alkyl.
Paragraph 134. The compound of any one of paragraphs 113-131, wherein R¹ is an alcohol-protecting group, an amino-protecting group, or a carboxylic acid protecting group.
Paragraph 135. The compound of paragraph 113, wherein the compound of Formula (A) is selected from any one of the following compounds:

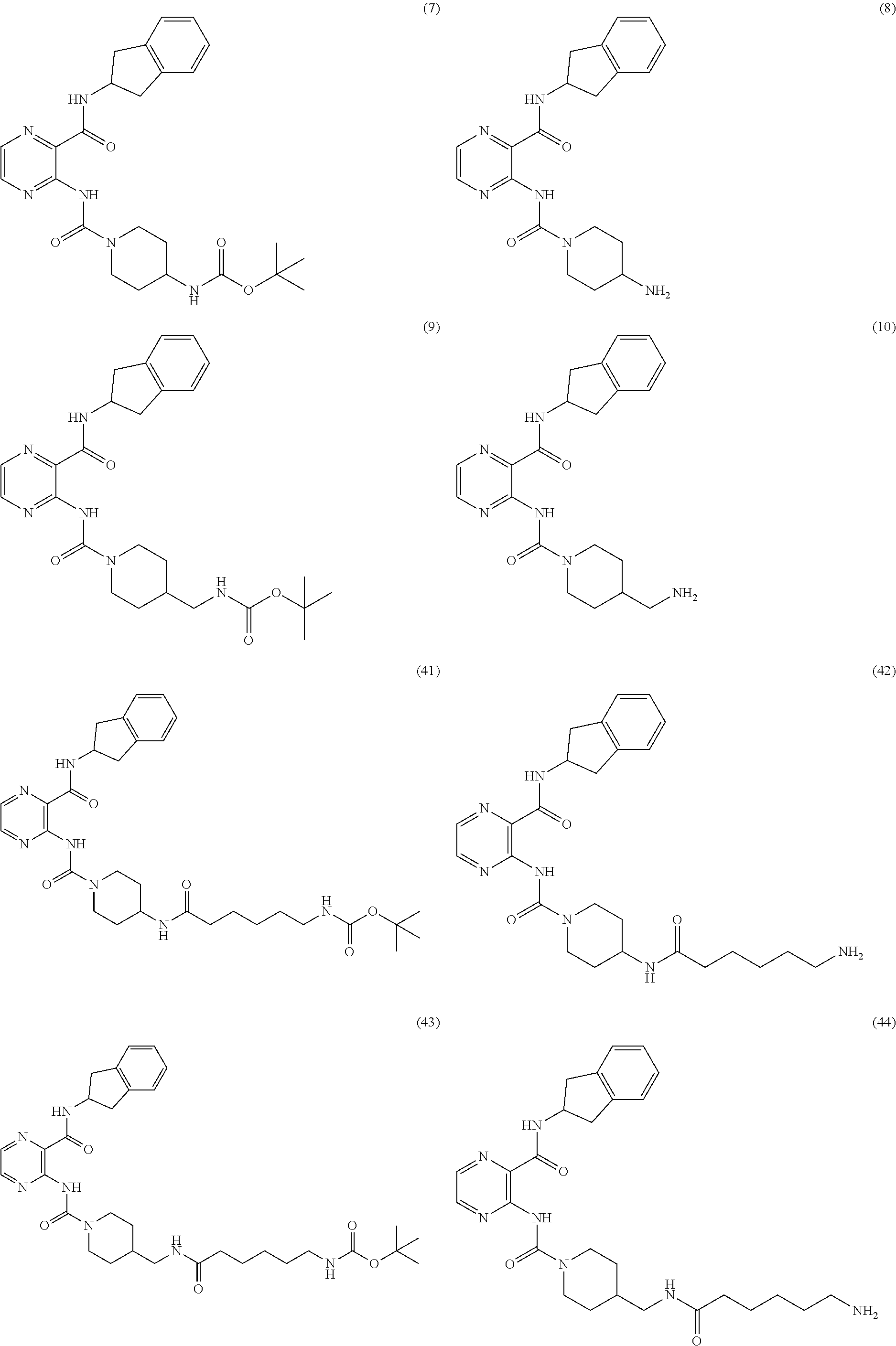

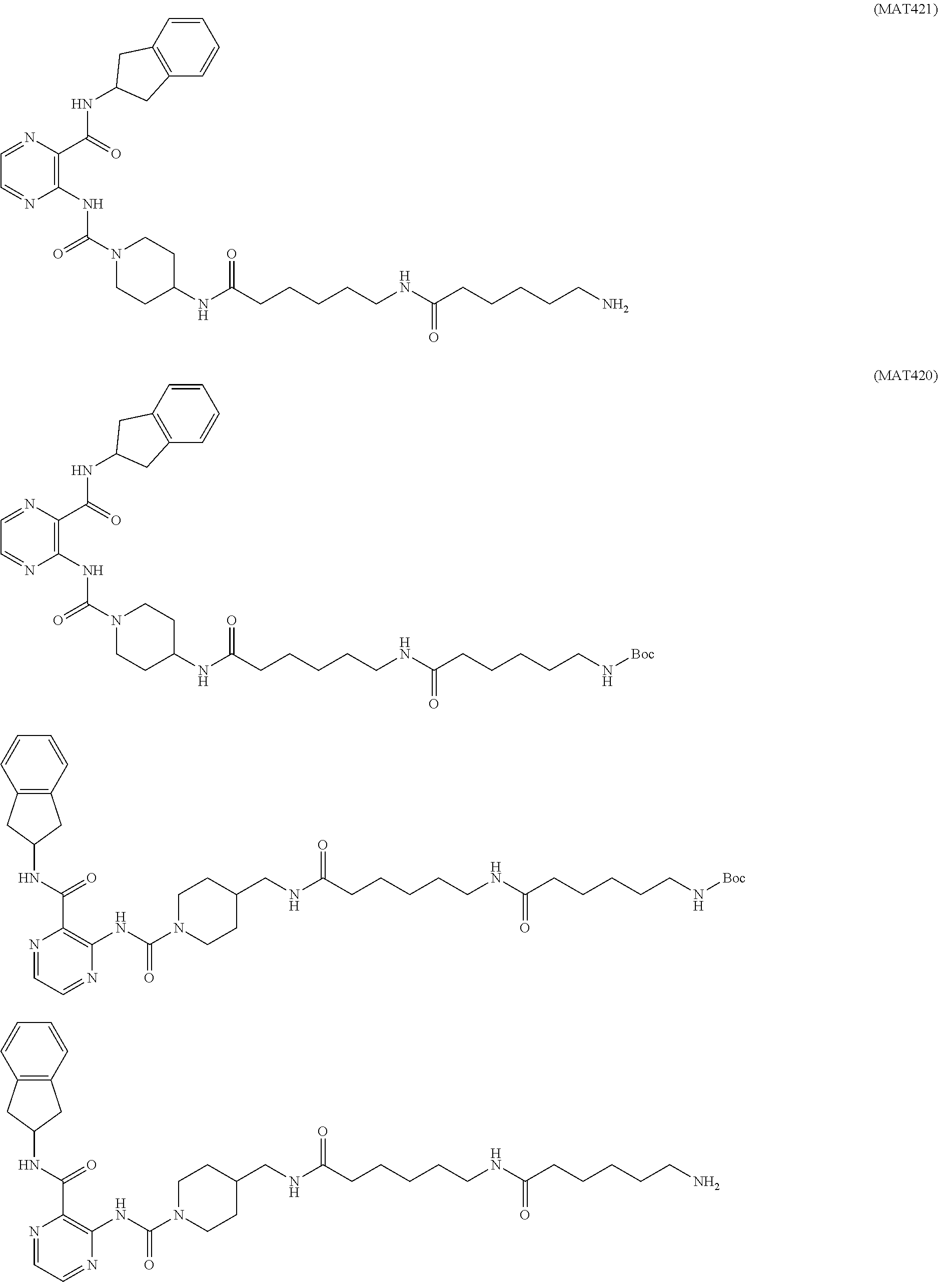

or a pharmaceutically acceptable salt thereof.
Paragraph 136. A composition comprising a compound of any one of paragraphs 1-135, or a pharmaceutically acceptable salt thereof, and an inert carrier.
Paragraph 137. The composition of paragraph 136, which is an aqueous solution.
Paragraph 138. The composition of paragraph 136. wherein the inert carrier is a buffer solution.
Paragraph 139. A compound of Formula (B):

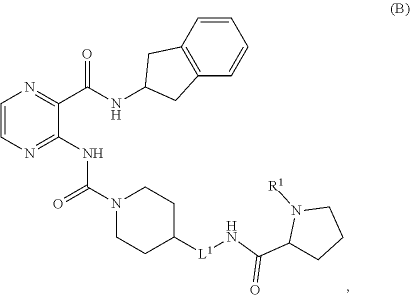

or a pharmaceutically acceptable salt thereof, wherein:
L¹ is C₁₋₃ alkylene; or L¹ is absent;
R¹ is selected from H, C₁₋₃ alkyl, C₁₋₃ haloalkyl, —C(═O)R^{A1}, and —C(═O)OR^{A1}; and
each R^{A1} is selected from H, C₁₋₆ alkyl, and C₁₋₃ haloalkyl.
Paragraph 140. The compound of paragraph 139, having formula:

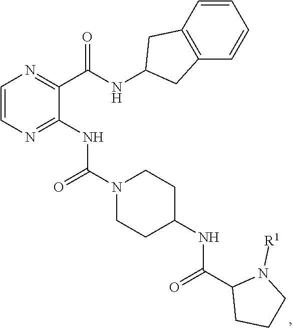

or a pharmaceutically acceptable salt thereof.
Paragraph 141. The compound of paragraph 139, wherein L¹ is C₁₋₃ alkylene.
Paragraph 142. The compound of paragraph 141, having formula:

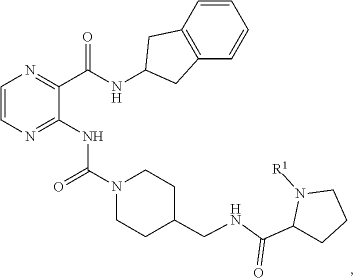

or a pharmaceutically acceptable salt thereof.
Paragraph 143. The compound of any one of paragraphs 139-142, wherein R¹ is H.
Paragraph 144. The compound of any one of paragraphs 139-142, wherein R¹ is C₁₋₃ alkyl.
Paragraph 145. The compound of any one of paragraphs 139-142, wherein R¹ is —C(═O)R^{A1}.
Paragraph 146. The compound of any one of paragraphs 139-142, wherein R¹ is —C(═O)OR^{A1}.
Paragraph 147. The compound of any one of paragraphs 139-142, wherein R^{A1} is H.
Paragraph 148. The compound of any one of paragraphs 137-142, wherein R^{A1} is C₁₋₆ alkyl.
Paragraph 149. The compound of paragraph 139, selected from any one of the following compounds:

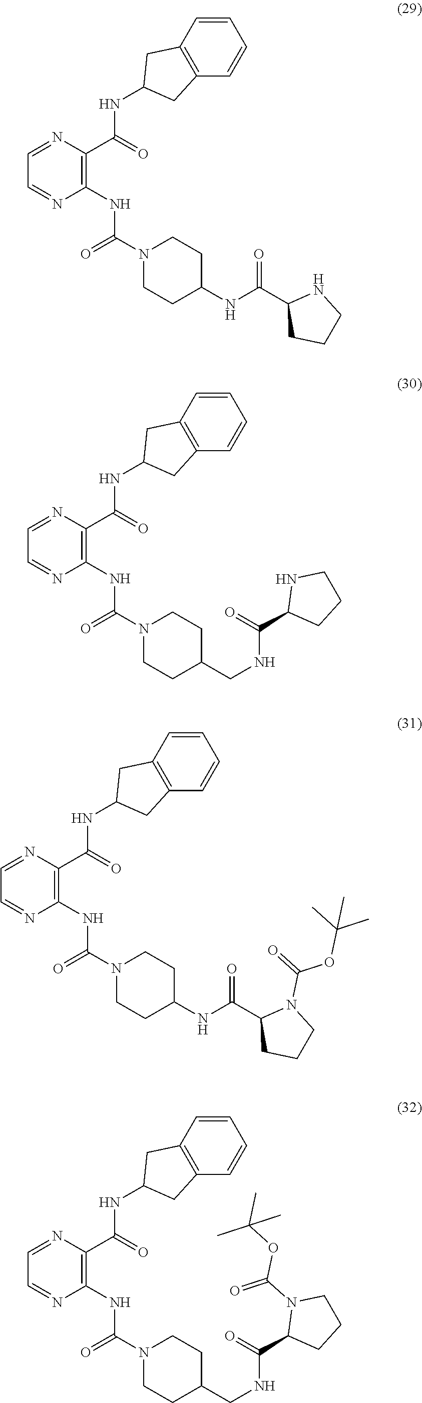 [SMILES: O=C(NC1Cc2ccccc2C1)c1nccnc1NC(=O)N1CCC(NC(=O)[C@@H]2CCCN2)CC1 ; O=C(NC1Cc2ccccc2C1)c1nccnc1NC(=O)N1CCC(CNC(=O)[C@@H]2CCCN2)CC1 ; CC(C)(C)OC(=O)N1CCC[C@H]1C(=O)NC1CCN(C(=O)Nc2nccnc2C(=O)NC2Cc3ccccc3C2)CC1 ; CC(C)(C)OC(=O)N1CCC[C@H]1C(=O)NCC1CCN(C(=O)Nc2nccnc2C(=O)NC2Cc3ccccc3C2)CC1]

or a pharmaceutically acceptable salt thereof.
Paragraph 150. A pharmaceutical composition comprising a compound of any one of paragraphs 139-149, or a pharmaceutically acceptable salt thereof, and a pharmaceutically acceptable carrier.
Paragraph 151. A method of inhibiting prolyl-tRNA-synthetase in a cell, comprising contacting the cell with a compound of any one of paragraphs 139-149, or a pharmaceutically acceptable salt thereof.
Paragraph 152. The method of paragraph 151, wherein the cell is a human cell or a protozoan parasitic cell.
Paragraph 153. The method of paragraph 152, the protozoan parasitic cell is selected from the group consisting of a Cryptosporidium, Babesia, Cyclospora, Cystoisospora, Toxoplasma, Giardia, and Plasmodia parasitic cell.
Paragraph 154. The method of paragraph 153, wherein the protozoan parasitic cell is selected a Plasmodia parasitic cell.
Paragraph 155. The method of paragraph 154, wherein the protozoan parasitic cell is selected from the group consisting of Plasmodium vivax, Plasmodium falciparum, Plasmodium malariae, Plasmodium ovale, and Plasmodium knowlesi.
Paragraph 156. The method of paragraph 154, wherein the protozoan parasitic cell is Plasmodium falciparum.
Paragraph 157. The method of paragraph 151, wherein the human cell is a cancer cell.
Paragraph 158. A method of inhibiting prolyl-tRNA-synthetase in a subject, comprising administering to the subject a compound of any one of paragraphs 139-149, or a pharmaceutically acceptable salt thereof.
Paragraph 159. A method of treating a disorder associated with glutamyl-prolyl-tRNA synthetase, prolyl-tRNA synthetase, or a combination thereof, the method comprising administering to a subject in need thereof a therapeutically effective amount of a compound of any one of paragraphs 139-149, or a pharmaceutically acceptable salt thereof, and a pharmaceutically acceptable carrier.
Paragraph 160. The method of paragraph 159, wherein the disorder is a parasitic infection.
Paragraph 161. The method of paragraph 160, wherein the parasitic infection is selected from malaria, toxoplasmosis, leishmaniasis, cryptosporidiosis, coccidiosis, Chagas disease, African sleeping sickness, giardiasis, and babesiosis.
Paragraph 162. The method of paragraph 161, wherein the disorder is malaria.
Paragraph 163. The method of paragraph 159, wherein the disorder is an autoimmune disease.
Paragraph 164. The method of paragraph 163, wherein the autoimmune disease is selected from multiple sclerosis, rheumatoid arthritis, lupus, psoriasis, scleroderma, dry eye syndrome, Crohn's Disease, inflammatory bowel disease, chronic obstructive pulmonary disease (COPD), asthma, fibrosis, scar formation, ischemic damage, and graft versus host disease.
Paragraph 165. The method of paragraph 159, wherein the disorder is a bacterial infection.
Paragraph 166. The method of paragraph 159, wherein the disorder is a fungal infection.
Paragraph 167. The method of paragraph 159, wherein the disorder is a viral infection.
Paragraph 168. The method of paragraph 167, wherein infection caused by corona virus, dengue virus and chikungunya virus.
Paragraph 169. The method of paragraph 159, wherein the disorder is selected from neurological disorder, a genetic disorder, a cardiovascular disorder, a protein aggregation disorder, a metabolic disorder, an inflammatory disorder, and a cosmetic disorder.
Paragraph 170. The method of paragraph 169, wherein the genetic disorder is Duchenne muscular dystrophy.
Paragraph 171. The method of paragraph 169, wherein the metabolic disorder is selected from diabetes and obesity.
Paragraph 172. The method of paragraph 169, wherein the cosmetic disorder is selected from the group consisting of cellulite and stretch marks.
Paragraph 173. The method of paragraph 159, wherein the inflammatory disorder is selected from restenosis, macular degeneration, choroidal neovascularization, chronic inflammation
Paragraph 174. The method of paragraph 159, wherein the disorder is cancer.
Paragraph 175. The method of paragraph 174, wherein the cancer is a T-cell neoplasm selected from mature T-cell leukemia, nodal peripheral T-cell lymphoma (PTCL), extranodal PTCLs, and cutaneous T-cell lymphoma (CTCL).
Paragraph 176. The method of paragraph 174, wherein the cancer is selected from adrenocortical carcinoma, bladder urothelial carcinoma, breast invasive carcinoma, cervical squamous cell carcinoma and endocervical adenocarcinoma, cholangio carcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine corpus endometrial carcinoma, uterine carcinosarcoma, uveal melanoma, multiple myeloma, and chordoma.
Paragraph 177. A method of identifying a compound that modulates a protein of interest, the method comprising:
   (i) providing a sample comprising a protein of interest attached to a FRET donor moiety and a ligand attached to a FRET acceptor moiety, said ligand capable of binding to the protein of interest;
   (ii) exciting the sample with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique;
   (iii) contacting the sample with a test compound:
   (iv) after (iii), detecting a light of a wavelength emitted by the FRET acceptor moiety by a fluorescence imaging technique; and
   (v) determining whether the intensity of fluorescence detected from the FRET acceptor moiety in step (iv) is decreased compared to the intensity of fluorescence detected from the FRET acceptor moiety in step (ii), wherein said decrease in fluorescence intensity in an indication that the test compound is the modulator the protein of interest.
Paragraph 178. The method of paragraph 177, wherein the compound is an inhibitor of a protein of interest.
Paragraph 179. The method of paragraph 177, wherein the compound is an activator of a protein of interest.
Paragraph 180. The method of paragraph 177, wherein the method is a high-throughput screening method.
Paragraph 181. The method of paragraph 177, wherein affinity of the ligand attached to the FRET acceptor moiety to the protein of interest is less than affinity of the test compound to the protein of interest.
Paragraph 182. The method of any one of paragraphs 177-181, wherein the wavelength of the light capable of being absorbed by the FRET donor moiety is from about 300 nm to about 400 nm.
Paragraph 183. The method of any one of paragraphs 177-182, wherein the wavelength emitted by the FRET acceptor moiety is from about 450 nm to about 600 nm.
Paragraph 184. The method of any one of paragraphs 177-183, wherein said detecting of fluorescence is carried out using fluorescent microscopy, fluorescent imaging probe, or fluorescent spectroscopy.
Paragraph 185. The method of any one of paragraphs 177-184, wherein the fluorescent FRET acceptor moiety is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.
Paragraph 186. The method of paragraph 185, wherein the fluorescent FRET acceptor moiety has formula:

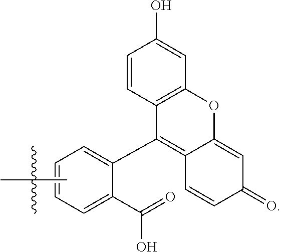

Paragraph 187. The method of any one of paragraphs 177-186, wherein the FRET donor moiety comprises a complex of a lanthanide metal with a moiety of formula (i):

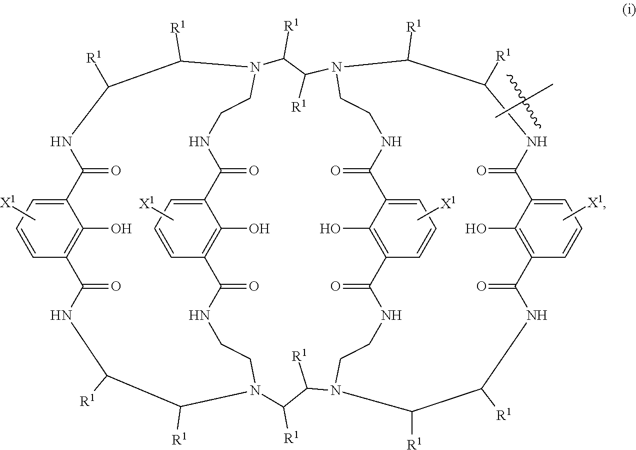

wherein:
each X¹ is independently selected from halo, NO₂, CN, N₃, C₁₋₆ alkyl, C₁₋₆ alkoxy, C₂₋₆ alkenyl, C₂₋₆ alkynyl, C₆₋₁₀aryl, and 5-14 membered heteroaryl, wherein said C₁₋₆ alkyl, C₂₋₆ alkenyl, C₂₋₆ alkynyl, C₆₋₁₀aryl, and 5-14 membered heteroaryl are each optionally substituted with 1, 2, or 3 substituents independently selected from halo, OH, SH, NH₂, C₁₋₃ alkylamino, di(C₁₋₃ alkyl)amino, NO₂, CN, C(O)OH, C₁₋₃ alkoxy, C₁₋₃ haloalkoxy, and N₃; and each R¹ is independently selected from H, C-6 alkyl, C₂₋₆ alkenyl, and C₂₋₆ alkynyl, each of which is optionally substituted with 1, 2, or 3 substituents independently selected from halo, OH, SH, NH₂, C₁₋₃ alkylamino, di(C₁₋₃ alkyl)amino, NO₂, CN, C(O)OH, C₁₋₃ alkoxy, C₁₋₃ haloalkoxy, and N₃.
Paragraph 188. The method of paragraph 187, wherein each X¹ is independently a H or a halo.
Paragraph 189. The method of paragraph 188, wherein each X¹ is H.
Paragraph 190. The method of paragraph 188, wherein each X¹ is C₁ or Br.
Paragraph 191. The method of any one of paragraphs 189-192, wherein one of R¹ is selected from C₁₋₆ alkyl, C₂₋₆ alkenyl, and C₂₋₆ alkynyl, each of which is optionally substituted with 1, 2, or 3 substituents independently selected from halo, OH, SH, NH₂, C₁₋₃ alkylamino, di(C₁₋₃ alkyl)amino, NO₂, CN, C(O)OH, C₁₋₃ alkoxy, C₁₋₃ haloalkoxy, and N₃; and the remaining R¹ groups are all H.
Paragraph 192. The method of any one of paragraphs 187-190, wherein each R¹ is H.
Paragraph 193. The method of paragraph 187, wherein the moiety of formula (i) has formula:

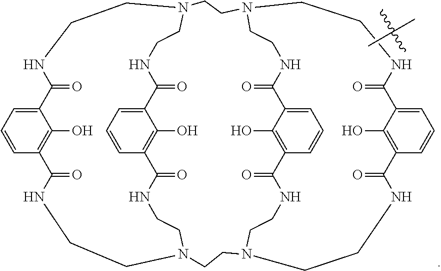

Paragraph 194. The method of paragraph 187, wherein the moiety of formula (i) has formula:

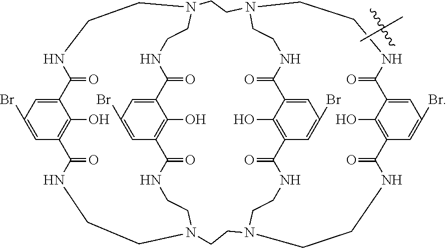

Paragraph 195. The method of paragraph 187, wherein the moiety of formula (i) has formula:

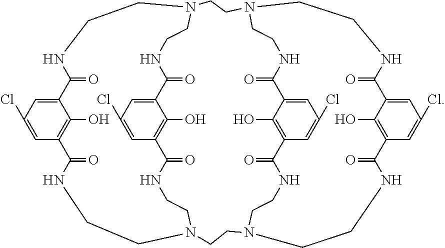 [SMILES: O=C1NCCN2CCNC(=O)c3cc(Cl)cc(c3O)C(=O)NCCN(CCNC(=O)c3cc(Cl)cc1c3O)CCN1CCNC(=O)c3cc(Cl)cc(c3O)C(=O)NCCN(CCNC(=O)c3cc(Cl)cc(c3O)C(=O)NCC1)CC2]

Paragraph 196. The method of any one of paragraphs 187-195, wherein the lanthanide metal is selected from Tb (terbium), Eu (europium), Sm (samarium), and Dy (dysprosium).
Paragraph 197. The method of paragraph 196, wherein the lanthanide metal is Tb³⁺.
Paragraph 198. The method of any one of paragraphs 187-197, wherein the protein of interest is selected from an enzyme, a cell-surface receptor, nuclear hormone receptor, a transporter, a G-protein coupled receptor, a CD marker, a voltage-gated ion channel, a nuclear factor, a nuclear receptor, a protein-protein or protein-peptide interaction domain, scaffolding protein, structural protein, transcription factor, chaperone, and assembly/disassembly factor.
Paragraph 199. The method of paragraph 198, wherein the enzyme is selected from kinases, proteases, deacetylases, ATPases, GTPases, phosphatases, peptidases, synthetases, phosphorilases, and nucleosidases.
Paragraph 200. The method of paragraph 199, wherein the protein of interest is selected from KEAP1 protein, bromodomain protein, and an aminoacyl tRNA synthetase.
Paragraph 201. The method of any one of paragraphs 177-1200, comprising making the protein of interest attached to a FRET donor moiety by contacting the protein of interest comprising a halotag with a FRET donor moiety comprising a halotag ligand.
Paragraph 202. The method of any one of paragraphs 177-201, comprising making the protein of interest attached to a FRET donor moiety by contacting the protein of interest comprising an epitope tag with an antibody or nanobody to the epitope tag, the antibody or nanobody being attached to the FRET donor moiety.
Paragraph 203. The method of any one of paragraphs 177-201, comprising making the protein of interest attached to a FRET donor moiety by contacting the protein of interest with an antibody or nanobody to the protein of interest, the antibody or nanobody being attached to the FRET donor moiety.
Paragraph 204. The method of any one of paragraphs 177-201, comprising making the protein of interest attached to a FRET donor moiety by contacting the protein of interest with a first antibody or nanobody to the protein of interest to obtain the protein-antibody conjugate, followed by contacting the conjugate with a second antibody or nanobody to the first antibody or nanobody, the second antibody or nanobody being attached to the FRET donor moiety.
Paragraph 205. The method of any one of paragraphs 177-201, comprising making the protein of interest attached to a FRET donor moiety by contacting the protein of interest comprising a biotin moiety with a streptavidin protein attached to the FRET donor moiety.
Paragraph 206. The method of any one of paragraphs 177-201, comprising making the protein of interest attached to a FRET donor moiety by contacting the protein of interest with a FRET donor moiety comprising an activated ester.
Paragraph 207. The method of paragraph 206, wherein the activated ester is selected from N-hydroxysuccinimide. sulfo-N-hydroxysuccinimide, tetrafluorophenoxy, pentafluorophenoxy, and p-nitophenoxy.
Paragraph 208. The method of any one of paragraphs 177-207, comprising making the ligand attached to the FRET acceptor moiety by coupling the ligand with the FRET acceptor moiety using a linker moiety.
Paragraph 209. The method of any one of paragraphs 177-208, wherein the protein of interest is an aminoacyl tRNA synthetase and the ligand attached to the FRET acceptor moiety is a compound of any one of paragraphs 1-22, or a pharmaceutically acceptable salt thereof, wherein the amino acid in the compound of Formula (I) corresponds to the amino acid attached to the tRNA by the aminoacyl tRNA synthetase enzyme.
Paragraph 210. The method of any one of paragraphs 177-208, wherein the protein of interest is a prolyl tRNA synthetase and the ligand attached to the FRET acceptor moiety is a compound of any one of paragraphs 23-67, or a pharmaceutically acceptable salt thereof.
Paragraph 211. The method of any one of paragraphs 177-208, wherein the protein of interest is a phenylalanyl tRNA synthetase and the ligand attached to the FRET acceptor moiety is a compound of any one of paragraphs 68-82, or a pharmaceutically acceptable salt thereof.
Paragraph 212. The method of any one of paragraphs 177-208, wherein the protein of interest is a isoleucyl tRNA synthetase and the ligand attached to the FRET acceptor moiety is a compound of any one of paragraphs 83-97, or a pharmaceutically acceptable salt thereof.
Paragraph 213. The method of any one of paragraphs 177-208, wherein the protein of interest is a glutamyl-tRNA synthetase and the ligand attached to the FRET acceptor moiety is a compound of any one of paragraphs 98-112, or a pharmaceutically acceptable salt thereof.
Paragraph 214. The method of any one of paragraphs 177-213, wherein the sample comprises live cells.
Paragraph 215. The method of any one of paragraphs 177-213, wherein the sample comprises a cell lysate.
Paragraph 216. The method of any one of paragraphs 177-213, wherein the sample comprises an aqueous solution.
Paragraph 217. The method of paragraph 216, wherein the aqueous solution is a buffer solution.
Paragraph 218. A method of evaluating an interaction between a protein of interest and a modulator of the protein of interest, the method comprising:
   (i) providing a sample comprising the protein of interest attached to a FRET donor moiety and the modulator attached to a FRET acceptor moiety;
   (ii) exciting the sample with a light of a wavelength capable of being absorbed by the FRET donor moiety, followed by waiting an amount of time sufficient for energy transfer from the FRET donor moiety to the FRET acceptor moiety; and
   (iii) detecting a light of a wavelength emitted by the FRET acceptor moiety by a fluorescence imaging technique;
   wherein the fluorescence intensity detected from the FRET acceptor moiety is indicative of quality and/or quantity of the interaction between the modulator and the protein of interest.
Paragraph 219. The method of paragraph 218, comprising determining a thermodynamic binding constant between the modulator and the protein of interest.
Paragraph 220. The method of paragraph 218, comprising determining a kinetic binding constant between the modulator and the protein of interest.
Paragraph 221. The method of paragraph 218, comprising determining the mode of binding of the modulator to the protein of interest.
Paragraph 222. The method of paragraph 221, comprising determining whether the modulator is a substrate-competitive orthosteric inhibitor, substrate-noncompetitive orthosteric inhibitor, or allosteric inhibitor.
Paragraph 223. The method of paragraph 221, comprising determining whether the modulator is an activator of the protein of interest.
Paragraph 224. The method of paragraph 218, comprising determining binding affinity between the modulator and the protein of interest.
Paragraph 225. The method of paragraph 218, comprising making the modulator attached to a FRET acceptor moiety by coupling the modulator to the FRET acceptor moiety through a linker.
Paragraph 226. A method of determining an amount of a protein of interest in a sample, the method comprising:
   (i) providing the sample comprising the protein of interest attached to a FRET donor moiety and the modulator attached to a FRET acceptor moiety:
   (ii) exciting the sample with a light of a wavelength capable of being absorbed by the FRET donor moiety, followed by waiting an amount of time sufficient for energy transfer from the FRET donor moiety to the FRET acceptor moiety; and
   (iii) detecting a light of a wavelength emitted by the FRET acceptor moiety by a fluorescence imaging technique:
   wherein the fluorescence intensity detected from the FRET acceptor moiety is correlated with the amount of the protein of interest in the sample.
Paragraph 227. The method of paragraph 226, wherein the method comprises quantifying abundance of a post-translational modification of the protein of interest.
Paragraph 228. The method of paragraph 226, comprising determining intracellular concentration of the protein of interest.
Paragraph 229. The method of paragraph 226, comprising obtaining a sample comprising the protein of interest from a subject for diagnosing a disease or condition, wherein the amount of the protein of interest in the sample is indicative of the disease or condition.
Paragraph 230. The method of paragraph 226, comprising obtaining a sample comprising the protein of interest from a subject for monitoring treatment a disease or condition, wherein the amount of the protein of interest in the sample is indicative of efficacy of treatment of the disease or condition.
Paragraph 231. The method of paragraph 226. wherein the sample comprises live cells.
Paragraph 232. The method of paragraph 226, wherein the sample comprises cell lysate.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A compound of Formula (I): 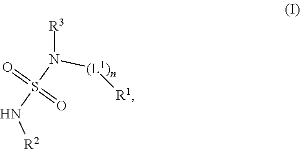
or a pharmaceutically acceptable salt thereof, wherein:
R³ is an ATP-binding moiety;
R² is an amino acid;
each L¹ is independently selected from O, S, S(═O)₂, NH, C═O, C═S, and C₁₋₆ alkylene;
n is an integer from 1 to 12; and
R¹ is a fluorophore.

2. The compound of claim 1, wherein the ATP-binding moiety is selected from any one of the following moieties: 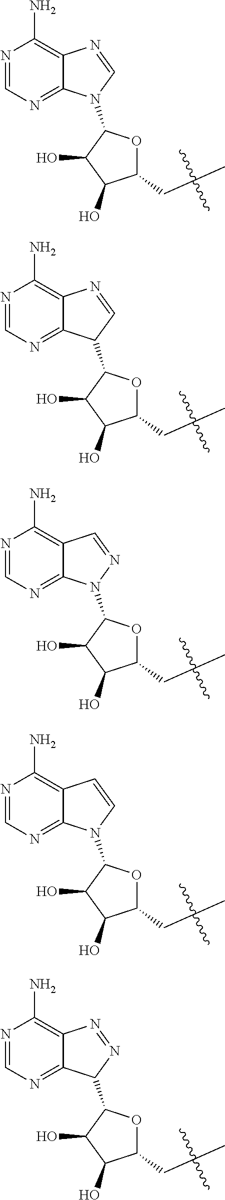 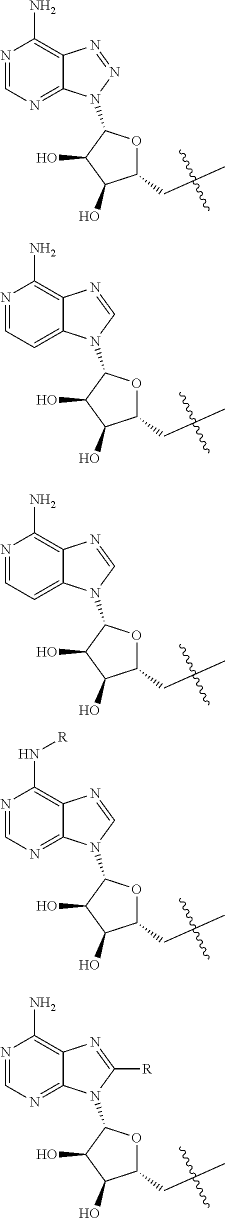 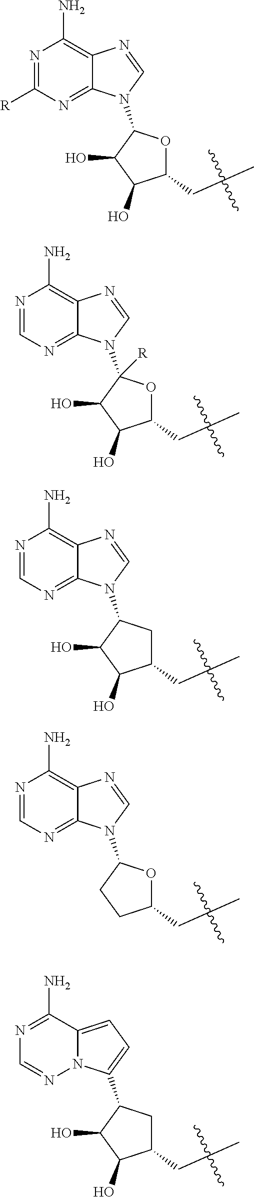 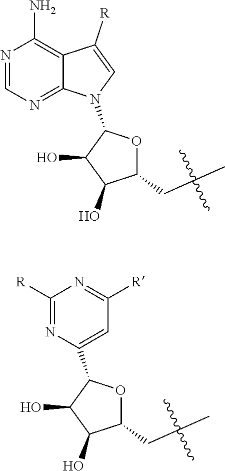
wherein R and R′ are independently selected from H, C₁₋₃ alkyl, and C₁₋₃ haloalkyl.

3. The compound of claim 1, wherein the compound has Formula (I): 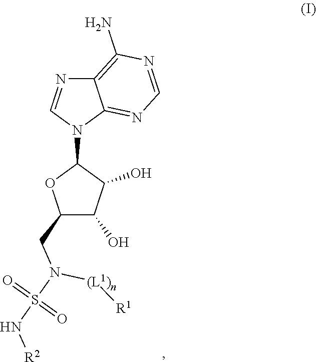
or a pharmaceutically acceptable salt thereof.

4. The compound of claim 1, where in the amino acid is selected from alanine, arginine, asparagine, aspartic acid, cysteine, glutamine, glutamic acid, glycine, histidine, isoleucine, leucine, isoleucine, lysine, methionine, phenylalanine, proline, serine, threonine, tryptophan, tyrosine, and valine.

5. A compound of Formula (II): 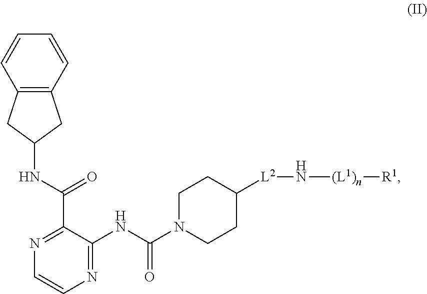
or a pharmaceutically acceptable salt thereof, wherein:
L² is C₁₋₃ alkylene; or L² is absent;
each L¹ is independently selected from O, S, S(═O)₂, NH, C═O, C═S, and C₁₋₆ alkylene;
n is an integer from 1 to 12; and
R¹ is a fluorophore.

6. A compound of Formula (IV): 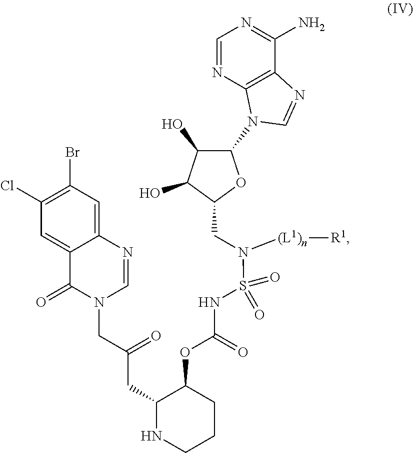
or a pharmaceutically acceptable salt thereof, wherein:
each L¹ is independently selected from O, S, S(═O)₂, NH, C═O, C═S, and C₁₋₆ alkylene;
n is an integer from 1 to 12; and
R¹ is a fluorophore.

7. A compound of Formula (A): 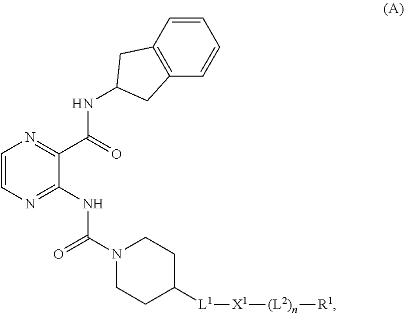
or a pharmaceutically acceptable salt thereof, wherein:
L¹ is C₁₋₃ alkylene, or L¹ is absent;
X¹ is selected from O and NR^{N};
R^{N} is selected from H, C₁₋₃ alkyl, and C₁₋₃ haloalkyl;
each L² is independently selected from O, S, S(═O)₂, NR^{N}, C═O, C═S, and C₁₋₆ alkylene;
n is an integer from 0 to 12; and
R¹ is selected from H, C₁₋₆ alkyl, and a protecting group.

8. A composition comprising a compound of claim 1, or a pharmaceutically acceptable salt thereof, and an inert carrier.

9. A method of identifying a compound that modulates a protein of interest, the method comprising:
(i) providing a sample comprising a protein of interest attached to a FRET donor moiety and a ligand attached to a FRET acceptor moiety, said ligand capable of binding to the protein of interest;
(ii) exciting the sample with a light of a wavelength capable of being absorbed by the FRET donor moiety, waiting an amount of time sufficient for energy transfer from the FRET donor moiety to the FRET acceptor moiety, and detecting a light of a wavelength emitted by the FRET acceptor moiety by a fluorescence imaging technique;
(iii) contacting the sample with a test compound;
(iv) after (iii), detecting a light of a wavelength emitted by the FRET acceptor moiety by a fluorescence imaging technique; and
(v) determining whether the intensity of fluorescence detected from the FRET acceptor moiety in step (iv) is decreased compared to the intensity of fluorescence detected from the FRET acceptor moiety in step (ii),
wherein said decrease in fluorescence intensity in an indication that the test compound is the modulator the protein of interest.

10. The method of claim 9, wherein the test compound is an inhibitor or an activator or the protein of interest.

11. The method of claim 9, wherein the method is a high-throughput screening method.

12. The method of claim 9, wherein said detecting of fluorescence is carried out using fluorescent microscopy, fluorescent imaging probe, or fluorescent spectroscopy.

13. The method of claim 9, wherein the fluorescent FRET acceptor moiety is selected from fluorescein, AF488, hydroxycoumarin blue, methoxycoumarin blue, alexa fluor blue, aminocoumarin blue, Cy2 green (dark), FAM green (dark), alexa fluor 488 green (light), fluorescein FITC green (light), alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red.

14. The method of claim 9, wherein the protein of interest is selected from an enzyme, a cell-surface receptor, nuclear hormone receptor, a transporter, a G-protein coupled receptor, a CD marker, a voltage-gated ion channel, a nuclear factor, a nuclear receptor, a protein-protein or protein-peptide interaction domain, scaffolding protein, structural protein, transcription factor, chaperone, and assembly/disassembly factor.

15. The method of claim 9, wherein the protein of interest is an aminoacyl tRNA synthetase and the ligand attached to the FRET acceptor moiety is a compound of claim 1, or a pharmaceutically acceptable salt thereof, wherein the amino acid in the compound of Formula (I) corresponds to the amino acid attached to the tRNA by the aminoacyl tRNA synthetase enzyme.

16. A compound of Formula (B): 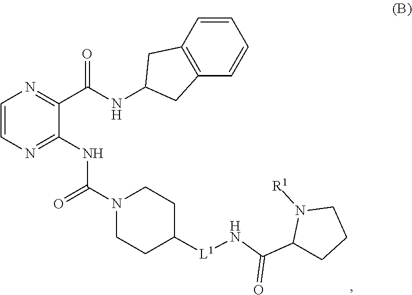
or a pharmaceutically acceptable salt thereof, wherein:
L¹ is C₁₋₃ alkylene; or L¹ is absent;
R¹ is selected from H, C₁₋₃ alkyl, C₁₋₃ haloalkyl, —C(═O)R^{A1}, and —C(═O)OR^{A1}; and
each R^{A1} is selected from H, C₁₋₆ alkyl, and C₁₋₃ haloalkyl.

17. A pharmaceutical composition comprising a compound of claim 16, or a pharmaceutically acceptable salt thereof, and a pharmaceutically acceptable carrier.

18. A method of inhibiting prolyl-tRNA-synthetase in a cell, comprising contacting the cell with a compound of claim 16, or a pharmaceutically acceptable salt thereof.

19. A method of treating a disorder associated with glutamyl-prolyl-tRNA synthetase, prolyl-tRNA synthetase, or a combination thereof, the method comprising administering to a subject in need thereof a therapeutically effective amount of a compound of claim 16, or a pharmaceutically acceptable salt thereof, and a pharmaceutically acceptable carrier.

20. The method of claim 19, wherein the disorder is selected from parasitic infection, autoimmune disease, bacterial infection, fungal infection, viral infection, neurological disorder, a genetic disorder, a cardiovascular disorder, a protein aggregation disorder, a metabolic disorder, an inflammatory disorder, a cosmetic disorder, and cancer.


SEQUENCE LISTING (18 sequences)

SEQ ID NO: 1 - DNA, 48 bp, synthetic construct
  source 1..48: mol_type = other DNA
gagccaaccactgaggatctgtactttcagagcgataacgcgatcgcc

SEQ ID NO: 2 - DNA, 22 bp, synthetic construct
  source 1..22: mol_type = other DNA
acatggcttagctgctactatt

SEQ ID NO: 3 - DNA, 31 bp, synthetic construct
  source 1..31: mol_type = other DNA
tccttagatttgttatcttctacttcattat

SEQ ID NO: 4 - DNA, 23 bp, synthetic construct
  source 1..23: mol_type = other DNA
tttaatcacgctcctgaatatgt

SEQ ID NO: 5 - DNA, 26 bp, synthetic construct
  source 1..26: mol_type = other DNA
aattgtttcacttgttggtcttatag

SEQ ID NO: 6 - DNA, 25 bp, synthetic construct
  source 1..25: mol_type = other DNA
ccgctgcatattatatatgggaatg

SEQ ID NO: 7 - DNA, 26 bp, synthetic construct
  source 1..26: mol_type = other DNA
agaaacatttggtggtagaactaatc

SEQ ID NO: 8 - DNA, 26 bp, synthetic construct
  source 1..26: mol_type = other DNA
tggtatgaagaatatctagctgtacc

SEQ ID NO: 9 - DNA, 25 bp, synthetic construct
  source 1..25: mol_type = other DNA
caacaagcatttgttgtgtttctaa

SEQ ID NO: 10 - DNA, 20 bp, synthetic construct
  source 1..20: mol_type = other DNA
gagccatacaagcagctaca

SEQ ID NO: 11 - DNA, 22 bp, synthetic construct
  source 1..22: mol_type = other DNA
ggtggttgatctaacgggatac

SEQ ID NO: 12 - DNA, 26 bp, synthetic construct
  source 1..26: mol_type = other DNA
gtttatgatgatagagcatcttattc

SEQ ID NO: 13 - DNA, 30 bp, synthetic construct
  source 1..30: mol_type = other DNA
gaaaaatggggtacatatatacaaatatat

SEQ ID NO: 14 - protein, 9 aa, synthetic construct
LDEETGEFL

SEQ ID NO: 15 - DNA, 4649 bp, synthetic construct
  source 1..4649: mol_type = other DNA
tcactgattaagcattggtaattcgaaatgaccgaccaagcgacgcccaaccggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatttcaagaagatcctttgatcttttctacggggtctgacgctcagtggaacgaaaactcacgttaagggattttggtcatgagattatcaaaaaggatcttcacctagatccttttatagtccggaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgattgcggcagtttttcgggtggtttgttgccatttttacctgtctgctgccgtgatcgcgctgaacgcgttttagcggtgcgtacaattaagggattatggtaaatccacttactgtctgccctcgtagccatcgagataaaccgcagtactccggccacgatgcgtccggcgtagaggatcgagatcttttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatggatctcgatcccgcgaaattaatacgactcactatagggtattttaactttactaaggagaattcatcatggcagaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctcgagatttccggcgagccaaccactgaggatctgtactttcagagcatcacaagtttgtacaaaaaagcaggctccaccatggacaaagactgcgaaatgaagcgcaccaccctggatagccctctgggcaagctggaactgtctgggtgcgaacagggcctgcaccgtatcatcttcctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctacagccacctggccgccctggccggcaatcccgccgccaccgccgccgtgaaaaccgccctgagcggaaatcccgtgcccattctgatcccctgccaccgggtggtgcagggcgacctggacgtggggggctacgagggcgggctcgccgtgaaagagtggctgctggcccacgagggccacagactgggcaagcctgggctgggtcaccatcaccatcaccattagacccagctttcttgtacaaagtggtgattaactagttgagcggccgcgcatgcaagctgatccggctgctaacaaagcccgaaaggaagctgagttggctgctgccaccgctgagcaataactagcataaccccttggggcctctaaacgggtcttgaggggttttttgctgaaaggaggaactatatccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtaatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcactttcaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaattcgaaatgaccgaccaagcgacgcccaacc

SEQ ID NO: 16 - DNA, 6126 bp, synthetic construct
  source 1..6126: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctggtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtattaatacgactcactatagggctagcgatcgccatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggccgtttctctcgagccaaccactgaggatctgtactttcagagcgataacgatggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggttaatagaattctagagtcgacctgcaggcatgcaagctgatccggctgctaacaaagcccgaaaggaagctgagttggctgctgccaccgctgagcaataactagcataaccccttggggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggtttttttaagcaagtaaaacctctacaaatgtggtaaaatcgaattctaatggatcctctttgcgcttgcgttttcccttgtccagatagcccagtagctgacattcatccggggtcagcaccgtttctgcggactggctttctacgtaatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcactttcaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaattcgaaatgaccgaccaagcgacgcccaaccggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatttcaagaagatcctttgatcttttctacggggtctgacgctcagtggaacgaaaactcacgttaagggattttggtcatgagattatcaaaaaggatcttcacctagatccttttatagtccggaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgattgcggcagtttttcgggtggtttgttgccatttttacctgtctgctgccgtgatcgcgctgaacgcgttttagcggtgcgtacaattaagggattatggtaaatccacttactgtctgccctcgtagccatcgagataaaccgcagtactccggccacgatgcgtccggcgtagaggatcgagatct

SEQ ID NO: 17 - protein, 575 aa, synthetic construct
EYVQIKEDTTSKARVDKKEDVQEEMAKNEELQNNNNNNKNNSNSNNNNNNNNNHIKDTILKGKLLSNNEVEDNKSKDSNILGITSKKIENFSDWYTQVIVKSELIEYYDISGCYILRPAAYYIWECVQAFFNKEIKKLNVENSYFPLFVTKNKLEKEKNHIEGFSPEVAWVTKYGDSNLPEEIAIRPTSETIMYSVFPKWIRSYRDLPLKLNQWNTVVRWEFKQPTPFIRTREFLWQEGHTAHKNEEEAVKLVFDILDLYRRWYEEYLAVPIIKGIKSEGEKFGGANFTSTAEAFISENGRAIQAATSHYLGTNFAKMFKIEFEDENEVKQYVHQTSWGCTTRSIGIMIMTHGDDKGLVLPPNVSKYKVVIVPIFYKTTDENAIHSYCKDIEKILKNAQINCVYDDRASYSPGYKFNHWELRGIPIRIEVGPKDLQNNSCVIVRRDNNEKCNVKKESVLLETQQMLVDIHKNLFLKAKKKLDDSIVQVTSFSEVMNALNKKKMVLAPWCEDIATEEEIKKETQRLSLNQTNSETTLSGAMKPLCIPLDQPPMPPNMKCFWSGKPAKRWCLFGRSY

SEQ ID NO: 18 - protein, 579 aa, synthetic construct
ELLQLKAQYKSLIGVEYKPVSATGAEDKDKKKKEKENKSEKQNKPQKQNDGQRKDPSKNQGGGLSSSGAGEGQGPKKQTRLGLEAKKEENLADWYSQVITKSEMIEYHDISGCYILRPWAYAIWEAIKDFFDAEIKKLGVENCYFPMFVSQSALEKEKTHVADFAPEVAWVTRSGKTELAEPIAIRPTSETVMYPAYAKWVQSHRDLPIKLNQWCNVVRWEFKHPQPFLRTREFLWQEGHSAFATMEEAAEEVLQILDLYAQVYEELLAIPVVKGRKTEKEKFAGGDYTTTIEAFISASGRAIQGGTSHHLGQNFSKMFEIVFEDPKIPGEKQFAYQNSWGLTTRTIGVMTMVHGDNMGLVLPPRVACVQVVIIPCGITNALSEEDKEALIAKCNDYRRRLLSVNIRVRADLRDNYSPGWKFNHWELKGVPIRLEVGPRDMKSCQFVAVRRDTGEKLTVAENEAETKLQAILEDIQVTLFTRASEDLKTHMVVANTMEDFQKILDSGKIVQIPFCGEIDCEDWIKKTTARDQDLEPGAPSMGAKSLCIPFKPLCELQPGAKCVCGKNPAKYYTLFGRSY